(19) United States
(12) Patent Application Publication
(10) Pub. No.: US 2011/0097360 A1
(43) Pub. Date: Apr. 28, 2011

(54) STREPTOCOCCUS PNEUMONIAE PILUS ANTIGENS

(76) Inventors: Claudio Donati, Poggibonsi (IT); Vega Masignani, Siena (IT); Marirosa Mora, Siena (IT); Monica Moschioni, Colle Val D' Elsa (IT)

(21) Appl. No.: 12/451,736

(22) PCT Filed: May 23, 2008
(86) PCT No.: PCT/IB2008/002108

§ 371 Date: May 16, 2010

Related U.S. Application Data
(60) Provisional application No. 60/940,167, filed on May 25, 2007.

(51) Int. Cl. A61K 39/09; C07K 2/00; C12N 9/00; C07K 14/315; C07K 4/04; C07H 21/00; C07H 21/04; C12N 1/20; A61P 37/04; A61P 31/04

(52) U.S. Cl. 424/242.1; 530/350; 435/183; 530/324; 530/326; 536/23.7; 536/23.1; 424/244.1; 435/253.4

(57) ABSTRACT

Polypeptides from Streptococcus pneumoniae are described. In some aspects the polypeptides include pili polypeptides from a second pili island (pilus II island (INV1 04B)) identified in Streptococcus pneumoniae isolate INV1 04. In other aspects the polypeptides include pili polypeptides and non-pilus polypeptides from Streptococcus pneumoniae strains 23F, INV200, and OXC141 that are absent from Streptococcus pneumoniae isolate INV104. The polypeptides, including fragments and variants thereof, may be used in immunogenic compositions for prophylactic or therapeutic immunization against Streptococcus pneumoniae. The polypeptides are also disclosed to be used in compositions useful for the production of antibodies and immunostimulants. Also presented are methods of inhibiting Streptococcus pneumoniae, methods of treating Streptococcus pneumoniae infection, methods of identifying inhibitors of Streptococcus pneumoniae and methods for diagnosing/detecting Streptococcus pneumoniae infection.

### FIELD

This invention relates to polypeptides, including pili proteins, from Streptococcus pneumoniae (S. pneumoniae), including fragments and variants thereof, and methods of their use in the treatment of and immunization against S. pneumoniae infections.

### BACKGROUND

The Gram-positive bacterium Streptococcus pneumoniae (also known as Spn or pneumococcus) is a major cause of morbidity and mortality world-wide and represents one of the four major infectious disease killers, together with HIV, malaria, and tuberculosis (Bruyn, G. A. W. & van Furth, R. (1991) Eur. J. Clin. Microbiol. Infect. Dis. 10, 897-910; Ryan, M. W. & Antonelli, P. J. (2000) Laryngoscope 110, 961-964; Cutts, F. T., Zaman, S. M., Enwere, G., Jaffar, S., Levine, O, S., Okoko, C. Oluwalana, A., Vaughan, S., Obaro, A., Leach, A., et al. (2005) Lancet 365, 1139-1146; Swiatlo, E., Champlin, F. R., Holman, S. C., Wilson, W. W.&Watt, J. M. (2002) Infect. Immun. 70, 412-415; Sandgren, A., Albiger, B., Orihuela, C., Tuomanen, E., Normark, S. & Henriques-Normark, B. (2005) J. Infect. Dis. 192, 791-800). It is a main cause of respiratory tract infections such as otitis media, sinusitis, and community acquired pneumonia, but also an important pathogen in invasive diseases such as septicemia and meningitis. Even though pneumococcus is a devastating pathogen, it also harmlessly colonizes healthy children attending day-care centers to a high extent (Henriques Normark, B., Christensson, B., Sandgren, A., Noreen, B., Sylvan, S., Burman, L. G. & Olsson-Liljequist, B. (2003) Microb. Drug Resist. 9, 337-344; Nunes, S., Sá-Leão, R., Carriço, J., Alves, C. R., Mato, R., Avô, A. B., Saldanha, J., Almeida, J. S., Sanches, I. S. & de Lencastre, H. (2005) J. Clin. Microbiol. 43, 1285-1293). A major virulence factor in pneumococcal disease is the polysaccharide capsule, by which pneumococci are grouped into at least ninety different serotypes (Henrichsen, J. (1995) J. Clin. Microbiol. 33, 2759-2762). Other genetic factors, such as CbpA (choline-binding protein A) and pneumolysin, have been described to be of importance for virulence (Lau, G. W., Haataja, S., Lonetto, M., Kensit, S. E., Marra, A., Bryant, A. P., McDevitt, D., Morrison, D. A. & Holden, D. W. (2001) Mol. Microbiol. 40, 555-571; Rosenow, C., Ryan, P., Weiser, J. N., Johnson, S., Fontan, P., Ortqvist, A. & Masure, H. R. (1997) Mol. Microbiol. 25, 819-829; Tuomanen, E. (1999) Current Opin. Biol. 2, 35-39).

Infection by S. pneumoniae leads to invasive disease triggered by initial colonization of the nasopharynx, but the mechanisms of adhesion are not well understood. In vitro adhesion of encapsulated pneumococci is much lower than for nonencapsulated nonvirulent derivatives (Swiatlo, E., Champlin, F. R., Holman, S. C., Wilson, W. W.&Watt, J. M. (2002) Infect. Immun. 70, 412-415), even though capsule expression is essential for successful colonization of the upper airways. These observations suggest that in vivo, pneumococci are adhesive despite the production of a thick capsule (Sandgren, A., Albiger, B., Orihuela, C., Tuomanen, E., Normark, S. & Henriques-Normark, B. (2005) J. Infect. Dis. 192, 791-800).

In other Gram-positive bacteria, such as Corynebacterium diphtheriae (Ton-That, H., Marraffini, L. A. & Schneewind, O. (2004) Mol. Microbiol. 53, 251-261; Ton-That, H. & Schneewind, O. (2003) Mol. Microbiol. 50, 1429-1438), Actinomyces spp. (Kelstrup, J., Theilade, J. & Fejerskov, O. (1979) Scand. J. Dent. Res. 87, 415-423), and recently group A streptococci (GAS) and group B streptococci (GBS) (Mora, M., Bensi, G., Capo, S., Falugi, F., Zingaretti, C., Manetti, A. G. O., Maggi, T., Taddei, A. R., Grandi, G. & Telford, J. L. (2005) Proc. Natl. Acad. Sci. USA 102, 15641-15646; Lauer, P., Rinaudo, C. D., Soriani, M., Margarit, I., Mainone, D., Rosini, R., Taddei, A. R., Mora, M., Rappuoli, R., Grandi, G. & Telford, J. L. (2005) Science 309, 105), pili-like surface structures have been identified by electron microscopy and characterized genetically as well as biochemically (Ton-That, H., Marraffini, L. A. & Schneewind, O. (2004) Mol. Microbiol. 53, 251-261; Ton-That, H. & Schneewind, O. (2003) Mol. Microbiol. 50, 1429-1438; Mora, M., Bensi, G., Capo, S., Falugi, F., Zingaretti, C., Manetti, A. G. O., Maggi, T., Taddei, A. R., Grandi, G. & Telford, J. L. (2005) Proc. Natl. Acad. Sci. USA 102, 15641-15646; Lauer, P., Rinaudo, C. D., Soriani, M., Margarit, I., Mainone, D., Rosini, R., Taddei, A. R., Mora, M., Rappuoli, R., Grandi, G. & Telford, J. L. (2005) Science 309, 105). In Actinomyces spp. type 1 fimbrial genes mediate adhesion to dental and mucosal surfaces (Li, T., Khah, M. K., Slavnic, S., Johansson, I. & Stromberg, N. (2001) Infect Immun. 69, 7224-7233). However, there is a need for functional data on the physiological role and function in infectious disease of pili and other antigens in pathogenic Streptococcus spp.

Gram-positive pili are extended polymers formed by a transpeptidase reaction involving covalent cross-linking of subunit proteins containing specific amino acid motifs, which are assembled by specific sortases. Sortases are also responsible for covalent attachment of the pilus to the peptidoglycan cell wall.

### SUMMARY

The present disclosure describes polypeptides from Streptococcus pneumoniae. In some aspects the polypeptides described herein include pili peptides from S. pneumoniae. In other aspects, other polypeptides from S. pneumoniae are described. The Streptococcus pneumoniae polypeptides described herein are useful in methods of treatment for and immunization against S. pneumoniae infections.

In some aspects, the disclosure features pili polypeptides from a second pili island identified in Streptococcus pneumoniae INV104B (pilus II island (INV104B)). In other aspects, the disclosure features pili polypeptides identified in S. pneumoniae 23F, INV200, and OXC141. The pili are thought to play a role in the pathogenesis of S. pneumoniae.

In some aspects, the disclosure features isolated pilus encoded by the Streptococcus pneumoniae pilus II island (INV104B). In some embodiments, the pilus includes a sortase. In some embodiments, the pilus includes an LPXTG cell wall anchored protein, e.g., a polypeptide having the amino acid sequence of SEQ ID NO:2, 4 and/or 6, or a processed form thereof.

In some embodiments, the pili are separated from cells by enzymatic digestion (e.g., with one or more enzymes such as peptidoglycan hydrolases (e.g., mutanolysin, lysostaphin, and lysozyme)). In some embodiments, the pili are separated from cells by mechanical shearing (e.g., by ultrasonication). In some embodiments, the pili are substantially free of bacterial cells. In some embodiments, the disclosure features methods of producing the pilus (e.g., S. pneumoniae pili), wherein the methods include subjecting a bacterial cell that produces the pilus to enzymatic digestion or mechanical shearing and isolating the pilus from the cell.

In other aspects, the disclosure features immunogenic compositions including more or more of the isolated pili (e.g., S. pneumoniae pili).

In other aspects, the disclosure features an isolated Streptococcus pneumoniae sortase, wherein the sortase is one of SEQ ID NO:282, SEQ ID NO:1386, SEQ ID NO:676, or SEQ ID NO:1123.

In other aspects, the disclosure features an isolated Streptococcus pneumoniae LPXTG cell wall anchored protein, wherein the LPXTG cell wall anchored protein is one of SEQ ID NO:2, SEQ ID NO:4, SEQ ID NO:6, SEQ ID NO:7, SEQ ID NO:8, or SEQ ID NO:9.

In further aspects, the disclosure features methods of isolating pili encoded by the Streptococcus pneumoniae pilus II island (INV104B), wherein the methods include subjecting bacterial cells that produce pili encoded by the Streptococcus pneumoniae pilus II island (INV104B) to enzymatic digestion (e.g., mutanolysin) or mechanical shearing (e.g., ultrasonication) and isolating the pili from the cells. In some embodiments, isolating includes a density gradient centrifugation. In some embodiments, isolating includes reduction of polydispersity, such as separating components by size, e.g., using gel filtration chromatography.

In other aspects, the disclosure features antibodies that bind specifically to a pilus encoded by the Streptococcus pneumoniae pilus II island (INV104B). In some embodiments, the antibody is a monoclonal antibody, a polyclonal antibody, a chimeric antibody, a human antibody, a humanized antibody, a single-chain antibody, or a Fab fragment.

In other aspects, the disclosure features an immunogenic composition comprising a purified Streptococcus pneumoniae pilus II island (INV104B) polypeptide in oligomeric form. In some embodiments, the polypeptide is a hyperoligomer. In other embodiments, the polypeptide is a fragment of a LPXTG cell wall anchored protein encoded by the Streptococcus pneumoniae pilus II island (INV104B).

In further aspects, the disclosure features methods of inducing an immune response against Streptococcus pneumoniae. In some embodiments, the methods comprise administering an effective amount of pili encoded by the Streptococcus pneumoniae pilus II island (INV104B) to a subject.

In other aspects, the disclosure features methods of detecting a Streptococcus pneumoniae infection in a subject. In some embodiments, the methods comprise assaying a sample from the subject for the presence of an antibody to pili encoded by the Streptococcus pneumoniae pilus II island (INV104B).

In other aspects, the disclosure features methods of detecting a Streptococcus pneumoniae infection in a subject. In some embodiments, the methods comprise contacting a sample with an antibody and detecting binding of the antibody to a component of the sample. In some embodiments, the antibody binds to a pili component. In other embodiments, the antibody binds to a pili complex.

In other aspects, the disclosure features methods of treating a subject having a Streptococcus pneumoniae infection. In some embodiments, the methods comprise administering to the subject an effective amount of an agent that binds specifically to pili encoded by the Streptococcus pneumoniae pilus II island (INV104B). In some embodiments, the agent is an antibody. In some embodiments, the antibody blocks attachment of Streptococcus pneumoniae to cells. In some embodiments, the antibody specifically binds to one or more LPXTG cell wall anchored proteins encoded by the Streptococcus pneumoniae pilus II island (INV104B).

In other aspects, the disclosure features methods of determining the course of treatment for a subject having a Streptococcus pneumoniae infection. In some embodiments, the method comprises assaying a sample from the subject for the presence of an antibody to pili encoded by the Streptococcus pneumoniae pilus II island (INV104B), and administering to the subject an anti-inflammatory agent if the presence of the antibody is detected. In other embodiments, the method comprises assaying a sample from the subject for the presence of an antibody to pili encoded by the Streptococcus pneumoniae pilus II island (INV104B), and administering to the subject an antibiotic agent if the presence of the antibody is not detected.

In other aspects, the disclosure features isolated pilus or pilus-like multimers that comprise an amino acid sequence of a pilus protein encoded by the Streptococcus pneumoniae pilus II island (INV104B) that has up to 30 amino acid substitutions, insertions, or deletions. In some embodiments, the amino acid sequence has up to 20 amino acid substitutions, insertions, or deletions. In other embodiments, the amino acid sequence has up to 10 amino acid substitutions, insertions, or deletions. In still other embodiments, amino acid sequence has up to 5 amino acid substitutions, insertions, or deletions.

In further aspects, the disclosure features polypeptides that have the amino acid sequence of one or more LPXTG cell wall anchored proteins encoded by the Streptococcus pneumoniae pilus II island (INV104B). In other aspects, the disclosure features immunogenic fragments of one or more LPXTG cell wall anchored proteins encoded by the Streptococcus pneumoniae pilus II island (INV104B). In other aspects, the disclosure features polynucleotides that encode polypeptides that have the amino acid sequence of one or more LPXTG cell wall anchored proteins encoded by the Streptococcus pneumoniae pilus II island (INV104B).

In other aspects, the disclosure features purified polypeptides with the amino acid sequence of SEQ ID NO:2, 4, 6, 7, 8, and 9. In other aspects, the disclosure features purified polypeptides having ten consecutive residues of SEQ ID NO:2, 4, 6, 7, 8, and 9. In still other aspects, the disclosure features purified polypeptides with amino acid sequences at least 85% identical to SEQ ID NO:2, 4, 6, 7, 8, and 9.

In additional aspects, the disclosure features purified polypeptides with at least 85% sequence identity to a sequence selected from the group consisting of SEQ ID NO:29 through SEQ ID NO:1742, or immunogenic fragments thereof. In other aspects, the disclosure features purified polypeptides with an amino acid sequence selected from the group consisting of SEQ ID NO:29 through SEQ ID NO:1742, or immunogenic fragments thereof.

In some embodiments, the disclosure features purified OCX141 polypeptides with at least 85% sequence identity to a sequence selected from the group SEQ ID NO: 53, SEQ ID NO: 65, SEQ ID NO: 70, SEQ ID NO: 99, SEQ ID NO: 104, SEQ ID NO: 117, SEQ ID NO: 135, SEQ ID NO: 177, SEQ ID NO: 178, SEQ ID NO: 198, SEQ ID NO: 235, SEQ ID NO: 236, SEQ ID NO: 237, SEQ ID NO: 242, SEQ ID NO: 247, SEQ ID NO: 248, SEQ ID NO: 250, SEQ ID NO: 251, SEQ ID NO: 252, SEQ ID NO: 253, SEQ ID NO: 433, SEQ ID NO: 439, SEQ ID NO: 444, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 581, or SEQ ID NO: 593, and immunogenic fragments thereof.

In other embodiments, the disclosure features purified INV200 polypeptides with at least 85% sequence identity to a sequence selected from the group SEQ ID NO: 626, SEQ ID NO: 628, SEQ ID NO: 629, SEQ ID NO: 630, SEQ ID NO: 631, SEQ ID NO: 632, SEQ ID NO: 639, SEQ ID NO: 645, SEQ ID NO: 747, SEQ ID NO: 751, SEQ ID NO: 752, SEQ ID NO: 783, SEQ ID NO: 786, SEQ ID NO: 787, SEQ ID NO: 810, SEQ ID NO: 812, SEQ ID NO: 813, SEQ ID NO: 824, SEQ ID NO: 831, SEQ ID NO: 842, SEQ ID NO: 847, SEQ ID NO: 875, SEQ ID NO: 876, SEQ ID NO: 879, SEQ ID NO: 880, SEQ ID NO: 882, SEQ ID NO: 913, SEQ ID NO: 914, SEQ ID NO: 925, SEQ ID NO: 926, SEQ ID NO: 947, SEQ ID NO: 948, SEQ ID NO: 968, SEQ ID NO: 987, SEQ ID NO: 988, SEQ ID NO: 990, SEQ ID NO: 992, SEQ ID NO: 1003, SEQ ID NO: 1007, SEQ ID NO: 1008, SEQ ID NO: 1036, SEQ ID NO: 1082, SEQ ID NO: 1120, or SEQ ID NO: 1123, and immunogenic fragments thereof.

In further embodiments, the disclosure features purified 23F polypeptides with at least 85% sequence identity to a sequence selected from the group SEQ ID NO: 1297, SEQ ID NO: 1309, SEQ ID NO: 1311, SEQ ID NO: 1343, SEQ ID NO: 1362, SEQ ID NO: 1364, SEQ ID NO: 1434, SEQ ID NO: 1451, SEQ ID NO: 1455, SEQ ID NO: 1466, SEQ ID NO: 14678, SEQ ID NO: 1470, SEQ ID NO: 1474, SEQ ID NO: 1484, SEQ ID NO: 1485, SEQ ID NO: 1486, SEQ ID NO: 1487, or SEQ ID NO: 1491, and immunogenic fragments thereof.

In other aspects, the disclosure features immunogenic fragments of an LPXTG cell wall anchored protein encoded by the Streptococcus pneumoniae pilus II island (INV104B).

In further aspects, the disclosure features isolated nucleic acids with the polynucleotide sequence of SEQ ID NO:1, 3, and 5. In other aspects, the disclosure features isolated nucleic acids that hybridize under stringent conditions to a hybridization probe, wherein the probe has the polynucleotide sequence of SEQ ID NO:1, 3, and 5 or the complement of SEQ ID NO:1, 3, and 5. In still other aspects, the disclosure features an isolated nucleic acid having a sequence that encodes an amino acid sequence that is at least 85% identical to SEQ ID NO:2, 4, 6, 7, 8, and 9.

In other aspects, the disclosure features isolated nucleic acids having a sequence that encodes an amino acid sequence that is at least 85% identical to a sequence selected from the group consisting of SEQ ID NO: 29 though SEQ ID NO:1742. In other aspects, the disclosure features isolated nucleic acids having a sequence that encodes an amino acid sequence that is selected from the group consisting of SEQ ID NO: 29 though SEQ ID NO:1742.

In other aspects, the disclosure features methods of inducing an immune response against Streptococcus pneumoniae. In some embodiments, the methods include administering an effective amount of an immunogenic fragment of a LPXTG cell wall anchored protein encoded by the Streptococcus pneumoniae pilus II island (INV104B) to a subject. In some embodiments, the subject is a human.

In other aspects, the disclosure features an antibody to a pilus protein encoded by the Streptococcus pneumoniae pilus II island (INV104B) in a cell. In some embodiments, the methods include expressing a nucleic acid encoding the antibody to the pilus protein encoded by the Streptococcus pneumoniae pilus II island (INV104B) in the cell. In some embodiments, the pilus protein is a LPXTG cell wall anchored protein.

In still other aspects, the disclosure features methods of purifying Streptococcus pneumoniae from a sample comprising Streptococcus pneumoniae. These methods include: providing an affinity matrix comprising an antibody bound to a solid support; contacting the sample with the affinity matrix to form an affinity matrix-Streptococcus pneumoniae complex; separating the affinity matrix-Streptococcus pneumoniae complex from the remainder of the sample; and releasing Streptococcus pneumoniae from the affinity matrix.

In further aspects, the disclosure features methods of delivering a cytotoxic agent or a diagnostic agent to Streptococcus pneumoniae. These methods include: providing the cytotoxic agent or the diagnostic agent conjugated to an antibody or fragment thereof; and exposing the Streptococcus pneumoniae to the antibody-agent or fragment-agent conjugate.

In other aspects, the disclosure features methods of identifying a binding modulator for pili encoded by the Streptococcus pneumoniae pilus II island (INV104B). These methods include contacting an animal cell susceptible to Streptococcus pneumoniae pili binding with a candidate compound and a bacterial cell having pili encoded by the Streptococcus pneumoniae pilus II island (INV104B), and determining whether binding of the bacterial cell to the animal cell is inhibited. In some embodiments, inhibition of the binding activity is indicative of an inhibitor of binding by pili encoded by the Streptococcus pneumoniae pilus II island (INV104B).

In other aspects, the disclosure features methods of identifying binding modulators to the activities of pili encoded by the Streptococcus pneumoniae pilus II island (INV104B). These methods include contacting an cell susceptible to Streptococcus pneumoniae pili binding with a candidate compound and a pili encoded by the Streptococcus pneumoniae pilus II island (INV104B), and determining whether binding of the pili to the cell is inhibited. In some embodiments, inhibition of binding activity is indicative of an inhibitor of binding by pili encoded by the Streptococcus pneumoniae pilus II island (INV104B).

In further aspects, the disclosure features methods of identifying a binding modulator for pili encoded by the Streptococcus pneumoniae pilus II island (INV104B). These methods include contacting an cell susceptible to Streptococcus pneumoniae pili binding with a candidate compound and a pilus protein or cell binding fragment thereof encoded by the Streptococcus pneumoniae pilus II island (INV104B), and determining whether binding of the pilus protein or cell binding fragment thereof to the cell is inhibited. In some embodiments, inhibition of binding activity is indicative of an inhibitor of binding by pili encoded by the Streptococcus pneumoniae pilus II island (INV104B).

In still other aspects, the disclosure features methods of isolating pili encoded by the Streptococcus pneumoniae pilus II island (INV104B). These methods include subjecting Streptococcus pneumoniae cells that produce pili encoded by the Streptococcus pneumoniae pilus II island (INV104B) to ultrasonication or digestion with a lytic enzyme; separating non-cellular components by density gradient centrifugation; and isolating pili encoded by the Streptococcus pneumoniae pilus II island (INV104B). In some embodiments, the lytic enzyme is mutanolysin. In other embodiments, the Streptococcus pneumoniae cells that produce pili encoded by the Streptococcus pneumoniae pilus II island (INV104B) are Streptococcus pneumoniae TIGR4 cells

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The details of one or more embodiments of the invention are set forth in the accompanying figures and the description below. Other features, objects, and advantages of the invention will be apparent from the description and figures, and from the additional embodiments below.

### BRIEF DESCRIPTION OF THE FIGURES

FIG. 1. is a diagram showing the S. pneumoniae pilus II island (INV104B).

### DESCRIPTION

The Applicants have identified new polypeptide sequences from Streptococcus pneumoniae (also known as pneumococcus), including new pili polypeptides and other polypeptides useful as antigens. New pili polypeptide sequences were identified in the unfinished genome sequence for S. pneumoniae isolate INV104, as sequenced by The Institute for Genomic Research (see worldwide web site “tigr.org”). These pili polypeptide sequences are encoded by a pathogenicity island, referred to herein as the pilus II island (INV104B), which is present in some, but not all, clinical pneumococcal isolates. Pili are important for pneumococcal adherence to lung epithelial cells as well as for colonization. Additionally, the Sanger partial genomes for S. pneumoniae strains 23F, INV200, and OXC141 were analyzed (see worldwide web site “sanger.ac.uk/Projects/Microbes/”) to identify genes encoding polypeptides absent from INV104, the results of which included additional pili polypeptides as well as a large number of other non-INV104 polypeptides. Accordingly, this disclosure features, inter alia, S. pneumoniae pili, pilus, and other polypeptide compositions and the use of the same in methods of treatment for, diagnosis of, and immunization against S. pneumoniae infections. As used herein the term S. pneumoniae Polypeptide(s) is meant to include S. pneumoniae pilus II island (INV104B) pili polypeptides, pili polypeptides from 23F, INV200, and OXC141, and other polypeptides from S. pneumoniae. Also as used herein the term S. pneumoniae Pilus Polypeptide(s) is meant to include S. pneumoniae pilus II island (INV104B) pili polypeptides and pili polypeptides from 23F, INV200, and OXC141.

S. pneumoniae Pili of the Pilus II Island of Inv104B

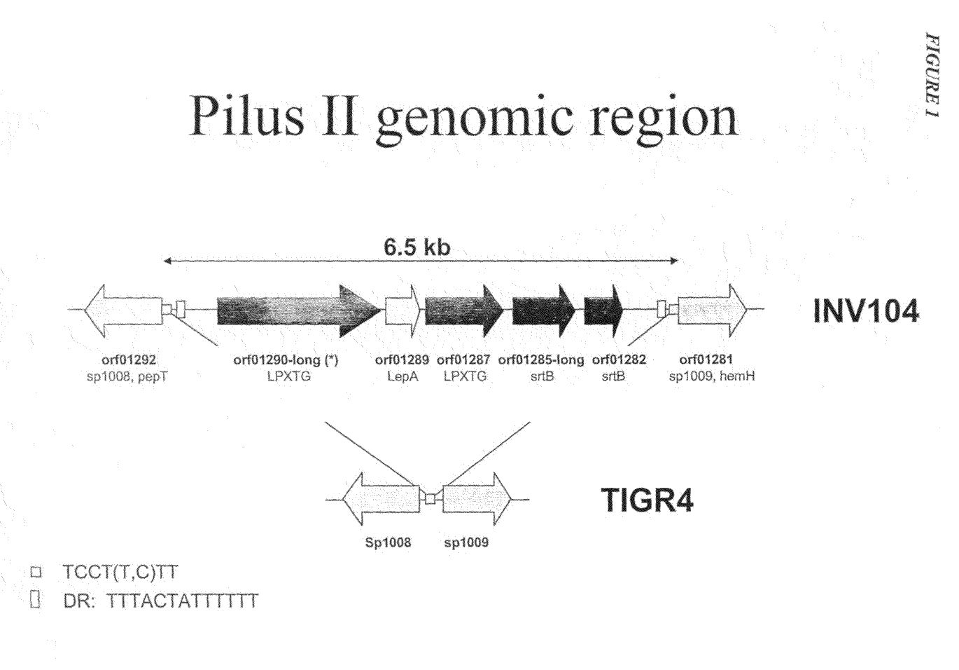

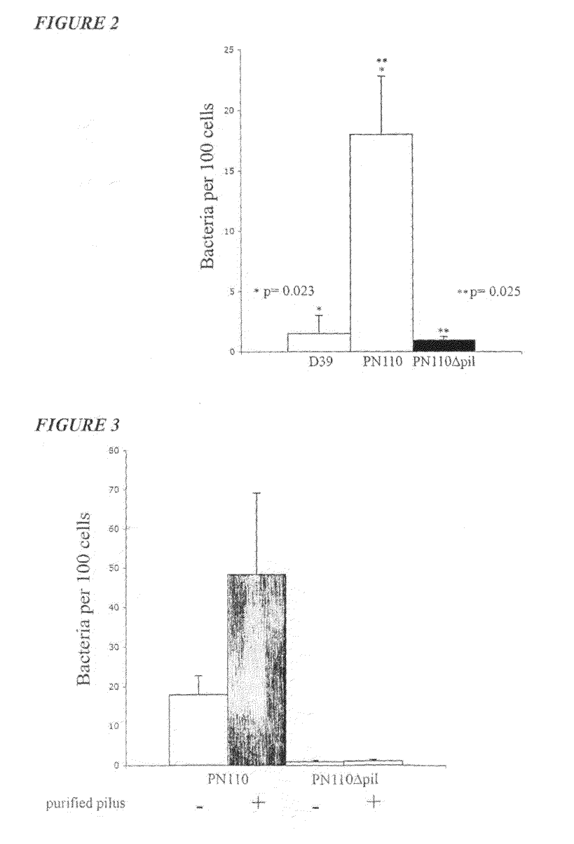

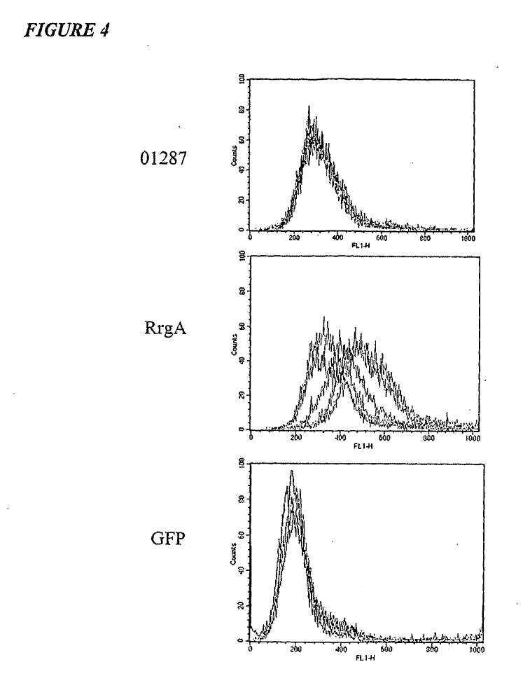

Pneumococcal pili encoded by a 6.5 kb insert between genes corresponding to sp1008 and sp1009 of S. pneumoniae isolate INV104B (ST227, serotype 1) of strain TIGR4 (as illustrated by FIG. 1) are described herein. This region of S. pneumoniae isolate INV104B is referred to herein as the “S. pneumoniae pilus II island (INV104B).” The S. pneumoniae pilus II island (INV104B) encodes three LPXTG cell wall anchored proteins (herein referred to as LPXTG-1, LPXTG-1A, and LPXTG-2), a LepA peptidase (orf01289; SEQ ID NO:673), and two predicted sortases (herein referred to as sort-1 (SEQ ID NO:676) and sort-2 (SEQ ID NO: 1123)). See Example 4 for these LepA and sortase sequences.

An exemplary nucleic acid sequence for LPXTG-1 (orf01290-long) is hereby provided:

(SEQ ID NO: 1)
ATGAACGTTCAATATGATTTTAAGAAGATTCAATATTTTACCAGTAGTTT
AGTTATCTTTCTCGCTATTCTTTTTTTGTGTGCACCAATTAATTCTTTAC
GTGCAGATTCAATAACTGAACCTCAGACAACTCTGCACAAAACGATTACT
CCGATATCAGGGCAAAAAGACCAGTATGAGTTGTCACTGGATATCACATC
TAAACTGGGAACGGAGACCCAGTCAGAACCCTTGGATGTAGTCTTGGTTG
CCGATCTTTCAGGGAGTATGGAAGAGCGAGATGTGTGGTCTTACTCTAGT
AGACGATACATTAGTAGGATTGAAGCACTAAAACATACACTGAAAGGTGT
GAATGGTCGTCAGGGGCTCATTGATACAATTCTTTCTAATTCCCAAAACC
GTCTGTCTATAGTTGGTTTTGCCGGAAAGATTGATAATCAGTATAATGAC
CGTTATTATAATGAATATTATCTGAGTTATCAATATGGAACTTGGCCAAA
TTGAGCTGGTTGGTATTCAAATATCTCTTCATATGATGATGCTAAAACTT
TAGTATCTTGGAGCACGGATTCTAATAGCTCAAAAAATATTGTTAGTTCG
TTAACAATTGCTGACTCTAGTCGTTCTTATGGTATGGACGCGGGCATTGG
CACTGGGACAAATATAAATGCTGGGTTAACTGAAGCTCAAAGATTGTTGC
AAAGTGCAAGGGCTGGGGCAAAAAAAGTAGTTATTCTGCTGTCAGATGGC
GAAGCTAATATGTATTACGAGTCTAATAGTGGGAGAACAATATATAACTA
TTATTCTAATCCAAATGTGGGACGTATGATTGATACTCCATATTGGTTTA
CCTCTGGTTTAGAGAGAGGAATGCTGAATATATCTAGTTTAATAGCTCCA
AAAATAGATGGCTTTTATTCAATCAAATTCAGATATATAGGTTCAAACGA
TAGTATCACATCTCTTAAAGGATATATCAGTGGTTATAATTCTGGAATCC
CCAACGAAATATTTTCTGCCAATAATGAAAATGACTTGCAACAAAAATTC
AAAGAAATCACAGATAAAATTCTACCTCTAGGCGTACACCATGTAACTAT
ATCAGATGTCTTGTCCAAGTACGTGCAGCTGTTACCTGGTGATGCTTCAC
ACCTTCGTGTCGTCAAAATCAAGGATGGTAACGAGCAAGAACTGAATGAC
AATCAAGTTACGATTGAAACTAAGAAGAACGAACAGGGATTAGTGGAAGT
AACAGCCAAGTTTAATCCGAGTTACACTTTGGAGGATGACGCCAAGTACG
TTCTCAAGTTTACTGTCACCTCTAGCCAAGAGGCATTTGATGCGATTGCG
GGTGATAAGACACTTACTAGTGATGATGCCGAAGAAGCCGATGCTACTAA
ACTCTACTCCAACAAGGGGGCAAAAGTTGCCTATTCCTATGGTATTGGGA
CCTCACGTACCAAAATAAAAGACTATTCTGAGAAGCCCACTTTCAAGCCG
TCAGATCCATTGACGGTTCCTGTAGAGATTGAGTGGAAAGGTGTGGATGG
AAAATCAAATCCATCAGCAAATCGTCCACCTAGTGTCGAATTAAACTTAA
ACCAAAAGAAAGATGGAAGTATAAAGGATTCCTATCGAAAGGTCACTAGT
CCAGTTCAAACGAATAGTTTTACTGAAAATACTAGTTTTGCAAAGGTAGC
TAAGGGATATGACTACGAACTGAAAGCACCAGACGCTCCGGGATACACAG
TCGAAGTTCAAAAGACAGGTACGAAAGAGAAACCATCCTTCAAAGTTATT
TACCGACAGCTTCCAAGTCTCACCGTAAAGAAAATCCTAGAAGGTGAACA
ATCACCTAATAAATCTTTCACAATTAATGTTACCTTTTCAGATAAGGATG
GCAAGCCGATTAACGGCAAGTTTGGGAATACAACAGTGACTAACGGGAAA
GCACAGATTTCTCTCAAAAATAGTCAGGAAACTGCCCTCAGTTATCTGCC
TCGTGATACCCACTATAAGGTGGAAGAAGTAGAGAACTCTAGAACGGGAT
ATCATGTCACCTATGAAAAACAAGAGGGGACTTTGTCAGAGGATGTTCAA
ACAATCGTCACCAACCACAGACTTCCGACACTTTCAGTCACAAAAAAAGT
TACAGGTGCTTTTGCTAATCTTCTGCAATCCTTTAAGATTACCATTAACG
TAAAGGATGCGCAAAATAAACCATTGAATGGATCGTATAGTGCAATAGTA
AATAATCAAAAAACAACGCTACAATTCACCAATGGTAAGGCGACAGTTGA
TCTAAAGAAAGATAAAACCATCAAGATTCTCGACCTTCCTCTAAATGCTC
GTTATAGTATCGAAGAAGAAGCAAGTTCGTCTCGTGGGTATCAGGTGTCC
TATGATAAAAAAGAAGGAACTCTTGATGCAAATAAGTCTGCGACAGTCAC
GAATAATAAAAACAGCGTACCTGAAACGGGAATTGACTTCTTGAGTAGCA
CTCTCGTGCTTGGAGTCGTTCTTCCTCTAGGAGGGATCTTCTTTATCATC
TTACTTGGTCACCTTGTGGTGAATAGGAGGAA

An exemplary amino acid sequence for LPXTG-1 is hereby provided:

(SEQ ID NOs: 769 and 2)
MNVQYDFKKIQYFTSSLVIFLAILFLCAPINSLRADSITEPQTTLHKTIT
PISGQKDQYELSLDITSKLGTETQSEPLDVVLVADLSGSMEERDVWSYSS
RRYISRIEALKHTLKGVNGRQGLIDTILSNSQNRLSIVGFAGKIDNQYND
RYYNEYYLSYQYGTWPN*AGWYSNISSYDDAKTLVSWSTDSNSSKNIVSS
LTIADSSRSYGMDAGIGTGTNINAGLTEAQRLLQSARAGAKKVVILLSDG
EANMYYESNSGRTIYVYYSNPNVGRMIDTPYWFTSGLERGMLNISSLIAP
KIDGFYSIKFRYIGSNDSITSLKGYISGYNSGIPNEIFSANNENDLQQKF
KEITDKILPLGVHHVTISDVLSKYVQLLPGDASHLRVVKIKDGNEQELND
NQVTIETKKNEQGLVEVTAKFNPSYTLEDDAKYVLKFTVTSSQEAFDAIA
GDKTLTSDDAEEADATKLYSNKGAKVAYSYGIGTSRTKIKDYSEKPTFKP
SDPLTVPVEIEWKGVDGKSNPSANRPPSVELNLNQKKDGSIKDSYRKVTS
PVQTNSFTENTSFAKVAKGYDYELKAPDAPGYTVEVQKTGTKEKPSFKVI
YRQLPSLTVKKILEGEQSPNKSFTINVTFSDKDGKPINGKFGNTTVTNGK
AQISLKNSQETALSYLPRDTHYKVEEVENSRTGYHVTYEKQEGTLSEDVQ
TIVTNHRLPTLSVTKKVTGAFANLLQSFKITINVKDAQNKPLNGSYSAIV
NNQKTTLQFTNGKATVDLKKDKTIKILDLPLNARYSIEEEASSSRGYQVS
YDKKEGTLDANKSATVTNNKNSVPETGIDFLSSTLVLGVVLPLGGIFFII
LLGHLVVNRRK

LPXTG-1 contains a sortase substrate motif, VPXTG (SEQ ID NO:16), shown in underscore in SEQ ID NO:2, above.

The orf01290-long sequence (SEQ ID NO:1) has an intermediary stop codon that is bolded and underscored in SEQ ID NO:1. An exemplary nucleic acid sequence halted at this stop codon (orf01290-short) would have the following transcribed gene sequence (LPXTG-1A):

(SEQ ID NO: 3)
ATGGACGCGGGCATTGGCACTGGGACAAATATAAATGCTGGGTTAACTGA
AGCTCAAAGATTGTTGCAAAGTGCAAGGGCTGGGGCAAAAAAAGTAGTTA
TTCTGCTGTCAGATGGCGAAGCTAATATGTATTACGAGTCTAATAGTGGG
AGAACAATATATAACTATTATTCTAATCCAAATGTGGGACGTATGATTGA
TACTCCATATTGGTTTACCTCTGGTTTAGAGAGAGGAATGCTGAATATAT
CTAGTTTAATAGCTCCAAAAATAGATGGCTTTTATTCAATCAAATTCAGA
TATATAGGTTCAAACGATAGTATCACATCTCTTAAAGGATATATCAGTGG
TTATAATTCTGGAATCCCCAACGAAATATTTTCTGCCAATAATGAAAATG
ACTTGCAACAAAAATTCAAAGAAATCACAGATAAAATTCTACCTCTAGGC
GTACACCATGTAACTATATCAGATGTCTTGTCCAAGTACGTGCAGCTGTT
ACCTGGTGATGCTTCACACCTTCGTGTCGTCAAAATCAAGGATGGTAACG
AGCAAGAACTGAATGACAATCAAGTTACGATTGAAACTAAGAAGAACGAA
CAGGGATTAGTGGAAGTAACAGCCAAGTTTAATCCGAGTTACACTTTGGA
GGATGACGCCAAGTACGTTCTCAAGTTTACTGTCACCTCTAGCCAAGAGG
CATTTGATGCGATTGCGGGTGATAAGACACTTACTAGTGATGATGCCGAA
GAAGCCGATGCTACTAAACTCTACTCCAACAAGGGGGCAAAAGTTGCCTA
TTCCTATGGTATTGGGACCTCACGTACCAAAATAAAAGACTATTCTGAGA
AGCCCACTTTCAAGCCGTCAGATCCATTGACGGTTCCTGTAGAGATTGAG
TGGAAAGGTGTGGATGGAAAATCAAATCCATCAGCAAATCGTCCACCTAG
TGTCGAATTAAACTTAAACCAAAAGAAAGATGGAAGTATAAAGGATTCCT
ATCGAAAGGTCACTAGTCCAGTTCAAACGAATAGTTTTACTGAAAATACT
AGTTTTGCAAAGGTAGCTAAGGGATATGACTACGAACTGAAAGCACCAGA
CGCTCCGGGATACACAGTCGAAGTTCAAAAGACAGGTACGAAAGAGAAAC
CATCCTTCAAAGTTATTTACCGACAGCTTCCAAGTCTCACCGTAAAGAAA
ATCCTAGAAGGTGAACAATCACCTAATAAATCTTTCACAATTAATGTTAC
CTTTTCAGATAAGGATGGCAAGCCGATTAACGGCAAGTTTGGGAATACAA
CAGTGACTAACGGGAAAGCACAGATTTCTCTCAAAAATAGTCAGGAAACT
GCCCTCAGTTATCTGCCTCGTGATACCCACTATAAGGTGGAAGAAGTAGA
GAACTCTAGAACGGGATATCATGTCACCTATGAAAAACAAGAGGGGACTT
TGTCAGAGGATGTTCAAACAATCGTCACCAACCACAGACTTCCGACACTT
TCAGTCACAAAAAAAGTTACAGGTGCTTTTGCTAATCTTCTGCAATCCTT
TAAGATTACCATTAACGTAAAGGATGCGCAAAATAAACCATTGAATGGAT
CGTATAGTGCAATAGTAAATAATCAAAAAACAACGCTACAATTCACCAAT
GGTAAGGCGACAGTTGATCTAAAGAAAGATAAAACCATCAAGATTCTCGA
CCTTCCTCTAAATGCTCGTTATAGTATCGAAGAAGAAGCAAGTTCGTCTC
GTGGGTATCAGGTGTCCTATGATAAAAAAGAAGGAACTCTTGATGCAAAT
AAGTCTGCGACAGTCACGAATAATAAAAACAGCGTACCTGAAACGGGAAT
TGACTTCTTGAGTAGCACTCTCGTGCTTGGAGTCGTTCTTCCTCTAGGAG
GGATCTTCTTTATCATCTTACTTGGTCACCTTGTGGTGAATAGGAGGAA

An exemplary amino acid sequence for LPXTG-1A (from orf01290-short) is hereby provided:

(SEQ ID NO: 4)
MDAGIGTGTNINAGLTEAQRLLQSARAGAKKVVILLSDGEANMYYESNSG
RTIYNYYSNPNVGRMIDTPYWFTSGLERGMLNISSLIAPKIDGFYSIKFR
YIGSNDSITSLKGYISGYNSGIPNEIFSANNENDLQQKFKEITDKILPLG
VHHVTISDVLSKYVQLLPGDASHLRVVKIKDGNEQELNDNQVTIETKKNE
QGLVEVTAKFNPSYTLEDDAKYVLKFTVTSSQEAFDAIAGDKTLTSDDAE
EADATKLYSNKGAKVAYSYGIGTSRTKIKDYSEKPTFKPSDPLTVPVEIE
WKGVDGKSNPSANRPPSVELNLNQKKDGSIKDSYRKVTSPVQTNSFTENT
SFAKVAKGYDYELKAPDAPGYTVEVQKTGTKEKPSFKVIYRQLPSLTVKK
ILEGEQSPNKSFTINVTFSDKDGKPINGKFGNTTVTNGKAQISLKNSQET
ALSYLPRDTHYKVEEVENSRTGYHVTYEKQEGTLSEDVQTIVTNHRLPTL
SVTKKVTGAFANLLQSFKITINVKDAQNKPLNGSYSAIVNNQKTTLQFTN
GKATVDLKKDKTIKILDLPLNARYSIEEEASSSRGYQVSYDKKEGTLDAN
KSATVTNNKNSVPETGIDFLSSTLVLGVVLPLGGIFFIILLGHLVVNRRK

LPXTG-1A contains a sortase substrate motif, VPXTG (SEQ ID NO:16), shown in underscore in SEQ ID NO:4, above.

An exemplary nucleic acid sequence for LPXTG-2 (orf01287) is hereby provided:

(SEQ ID NO: 5)
TTGATGATCATAATGAAAAAAGAAAATAAAAAAACAAAAGAAATAATCAT
GAAAAAAACATTCTTTAAAAAGCTATTCACTGCAAGCATTGCAGCTATAA
CCGCTTTGTCCGTATTCAGAGGTGTCCCGACTTTTGCGGATGATAATTCA
GCAATAACCAAAGCAAATGGTGAAAATAATGCTGTTGTGAAGATTAATAA
AACGTTGAATATTGCAGAGGGAATAACAACACCAACAGCGACATTTACAT
TTAAGTTTACAGAAAAAACAGGACAATCTTCTAACGGTGCGCCATATCAA
ACCGGAGTTGCAATTCCAGATAGAAATGTAGAATACAATAAAAATGATCA
CCCAACTGCTGATAAGATTCAAAAAGCAACAGAAGACATTTTTTCGGGAG
TTGCTTATGGCCATGCTGGTGAATACGTTTATGATGTAGCGGAAGCAAAA
ACTGGATGGCAGGCGATTACCAAAAATGGTAAAACAATTGATGCCATGAG
ATACGACAAACGTACATATGAAATGCACGTTATTGTTAAGAATAAAGTAA
ATGGTGGTGTCTATATTTCATCAGTATACTTTAAGGAAAATAATAAATCT
AACGCCCCTAAAGTAGAACCAAGTGAACAAGGCGTTTATAATTTATTTGA
TAACACATATACCAAAGACGCAAGTAAGGAGCCTAATCCTGATGATCCGA
GTCAAGTAGACCCCAATGCGAAAGCATTAACAATTACTAAAAAAGTTGAT
GGAGCTTCAGGGGATAAAACAAGAGATTTCCAATTCCATATCAAGATTCA
ACTTCCAAGTACAAATAAAACAGCAGAAACCCCTGTTACGAATATTATAG
TAAAACATGGATCTAAGTCAGAGGTGTTGGCAGTAGTGACCCCAGCAGAT
ACAGTTGAGTACAATTTTACTCTTAAAGATGGTGAAACATTTACAGTTGA
ACAACTACCAGCAGGTTCTAAATATACAGTAACTGAAACTGGAGTAGCAG
GTTATACAGATTCATCAATTTATACTACAAATGGTGCAGAACAAACATCT
CAAGGACAAAAAAATGTAGATTTTACATTAACAGATATCCTCATAGGTGA
AAAGAAAAACGACAACAAAGTTACTAACAAAATCGACGACGTTACTCCTA
CTGGTCTCTTGATTGATAACCTTCCATTCATTTTGATGATTGGTCTTGGT
TTGGCTGGATTTGTTGTCTTGTCTAAAAAACGTAGAGAAGCCTA

An exemplary amino acid sequence for LPXTG-2 is hereby provided:

(SEQ ID NO: 6)
MIIMKKENKKTKEIIMKKTFFKKLFTASIAAITALSVFRGVPTFADDNSA
ITKANGENNAVVKINKTLNIAEGITTPTATFTFKFTEKTGQSSNGAPYQT
GVAIPDRNVEYNKNDHPTADKIQKATEDIFSGVAYGHAGEYVYDVAEAKT
GWQAITKNGKTIDAMRYDKRTYEMHVIVKNKVNGGVYISSVYFKENNKSN
APKVEPSEQGVYNLFDNTYTKDASKEPNPDDPSQVDPNAKALTITKKVDG
ASGDKTRDFQFHIKIQLPSTNKTAETPVTNIIVKHGSKSEVLAVVTPADT
VEYNFTLKDGETFTVEQLPAGSKYTVTETGVAGYTDSSIYTTNGAEQTSQ
GQKNVDFTLTDILIGEKKNDNKVTNKIDDVTPTGLLIDNLPFILMIGLGL
AGFVVLSKKRREA

LPXTG-2 contains a sortase substrate motif, VTXTG (SEQ ID NO:21), shown in underscore in SEQ ID NO:6, above.

Polypeptide Sequences Identified in 23F, INV200, and OXC141 S. pneumoniae Strains

The Sanger partial genomes for S. pneumoniae strains 23F, INV200, and OXC141 do not contain the S. pneumoniae pilus II island (INV104B) region. However, 23F, INV200, and OXC141 encode sortases and LPXTG cell wall anchored proteins, which are disclosed herein. For example, 23F and OXC141 each encode at least one sortase (sort-23F (orf01917; SEQ ID NO:1386) and sort-OXC141 (orf01672; SEQ ID NO:282)) and INV200 encodes at least three cell wall anchored proteins (Anchor-1, Anchor-2, and Anchor-3) that are not encoded by S. pneumoniae strain INV104B.

An exemplary amino acid sequence for Anchor-1 (INV200-orf00426) is hereby provided:

(SEQ ID NO: 7)
MRVSSDTNIYEYRALSPQQKAALEMIRADLYKFTVPYENLEYRFYKPDWV
FGLGYQALATVRWKIEPATITVTKKWENVKEGAKKPDVWIQLLKDGKPEG
ERKRIESDKGQTTFEIPNKDEINKYSVKEVDKEGRDWKHKDFTAGQPVNK
GNGHFEITNTKKEKPKIKVTFKKIAGDTNKDLAGAHLVLKKIFDDGNGLL
IKQWDTIGQPVDIDLDAGSYTLTEEKAPDGYMLAAPVSFYVEEDGQIILP
KGEDLEAQNDKTITMVDEKIKEKPTKPSGKLATTVEVDGTKADAQKELEL
SVATDKVTKTVKDTVVYENLLAGETYKLTGQLMKITADKEEEVATKETTF
VADASGNGTTSLEFEDVSLEAGVKYVVYETAESEKEIDFKEGKEKHKVEH
KDKDDKAQTVVVTKEKPTKPSGKLATTVEVDGTKADAQKELELSVATDKV
TKTVKDTVVYENLLAGETYKLTGQLMKITADKEEEVATKETTFVADASGN
GTTSLEFEDVSLEAGVKYVVYETAESEKEIDFKEGKEKHKVEHKDKDDKA
QTVVVSKIKPEPGAQEVHFSKVNVGGEEIAGAEIHIKQGDTVVASWVSEA
GKTHTLKLKPGHYIFHEAVAPGGYLAVTDIHFSVDETGQVTVTDVNGNTA
VAEGNKLTVTDQTKPVTPPSPEEPGAQEVHFSKVNVGGEEIAGAEIHIKQ
GDTVVASWVSEAGKTHTLKLKPGHYIFHEAVAPGGYLAVTDIHFSVDETG
QVTVTDVNGNTAVAEGNKLTVTDQTKPVTPPSPEEPGAQEVHFSKVNVGG
EEIAGAEIHIKQGDTVVASWVSEAGKTHTLKLKPGHYIFHEAVAPGGYLA
VTDIHFSVDETGQVTVTDVNGNTAVAEGNKLTVTDQSADKDKQDKLPNTG
ETTGTYLSILGMITAVFASLLYRSKKK

Anchor-1 contains a sortase substrate motif, LPNTG (SEQ ID NO:10), shown in underscore in SEQ ID NO:7, above.

An exemplary amino acid sequence for Anchor-2 (INV200-orf00441) is hereby provided:

(SEQ ID NO: 8)
MNKGLFEKRCKYSIRKFSLGVASVMIGAAFFGTSPVLADSVQSGSTANLP
ADLATALATAKENDGRDFEAPKVGEDQGSPEVTDGPKTEEELLALEKEKP
AEEKPKEDKPAAAKPETPKTVTPEWQTVEKKEQKGTVTIREEKGVRYNQL
SSTAQNDNAGKPALFEKKGLTVVANGNATVDLTFKDDSEKGKSRFGVFLK
FKDTNNNVFVGYDKDGWFWEYKSPTTSTWYRGSRVAAPETGSTNRLSITL
KSDGQLNASNNDVNLFDTVTLPAAVNDHLKNEKKILLKAGSYGNDRTVVS
VKTDNQEGVKADDTPAQKETGPVVDDSEVTYDTIQSKVLKAVIDQAFPRV
KEYSLNGHTLPGQVQQFNQVFINNHRITPEVTYKKINETTAEYLMKIRDD
AHLINAEMTVRLQVVDNQLHFDVTKIVNHNQVTPGQKIDDERKLLSSISF
LGNALVSVSSDQTGAKFDGATMSNNTHVSGDDHIDVTNPMKDLAKGYMYG
FVSTDKLAAGVWSNSQNSYGGGSNDWTRLTAYKETVGNANYVGIHSSEWQ
WEKAYKGIVFPEYTKELPSAKVVITEDANADKKVDWQDGAIAYRSIMNNP
QGWEKVKDITAYRIAMNFGSQAQNPFLMTLDGIKKINLHTDGLGQGVLLK
GYGSEGHDSGHLNYADIGKRIGGVEDFKTLIEKAKKYGAHLGIHVNASET
YPESKYFNEKILRKNPDGSYSYGWNWLDQGINIDAAYDLAHGRLARWEDL
KKKLGDGLDFIYVDVWGNGQSGDNGAWATHVLAKEINKQGWRFAIEWGHG
GEYDSTFHHWAADLTYGGYTNKGINSAITRFIRNHQKDAWVGDYRSYGGA
ANYPLLGGYSMKDFEGWQGRSDYNGYVTNLFAHDVMTKYFQHFTVSKWEN
GTPVTMTDNGSTYKWTPEMRVELVDADNNKVVVTRKSNDVNSPQYRERTV
TLNGRVIQDGSAYLTPWNWDANGKKLSTDKEKMYYFNTQAGATTWTLPSD
WAKSKVYLYKLTDQGKTEEQELTVKDGKITLDLLANQPYVLYRSKQTNPE
MSWSEGMHIYDQGFNSGTLKHWTISGDASKAEIVKSQGANDMLRIQGNKE
KVSLTQKLTGLKPNTKYAVYVGVDNRSNAKASITVNTGEKEVTTYTNKSL
ALNYVKAYAHNTRRDNATVDDTSYFQNMYAFFTTGADVSNVTLTLSREAG
DQATYFDEIRTFENNSSMYGDKHDTGKGTFKQDFENVAQGIFPFVVGGVE
GVEDNRTHLSEKHNPYTQRGWNGKKVDDVIEGNWSLKTNGLVSRRNLVYQ
TIPQNFRFEAGKTYRVTFEYEAGSDNTYAFVVGKGEFQSGRRGTQASNLE
MHELPNTWTDSKKAKKATFLVTGAETGDTWVGIYSTGNASNTRGDSGGNA
NFRGYNDFMMDNLQIEEITLTGKMLTENALKNYLPTVAMTNYTKESMDAL
KEAVFNLSQADDDISVEEARAEIAKIEALKNALVQKKTALVADDFASLTA
PAQAQEGLANAFDGNVSSLWHTSWNGGDVGKPATMVLKEPTEITGLRYVP
RGSGSNGNLRDVKLVVTDESGKEHTFTATDWPDNNKPKDIDFGKTIKAKK
IVLTGTKTYGDGGDKYQSAAELIFTRPQVAETPLDLSGYEAALAKAQKLT
DKDNQEEVASVQASMKYATDNHLLTERMVEYFADYLNQLKDSATKPDAPT
VEKPEFKLSSLVSEQGKTPDYKQEIARPETPEQILPATGESQSDTSLFLA
SVSLALSALFVVKTKKD

Anchor-2 contains a sortase substrate motif, LPATG (SEQ ID NO:10), shown in underscore in SEQ ID NO:8, above.

An exemplary amino acid sequence for Anchor-3 (INV200-orf03448) is hereby provided:

(SEQ ID NO: 9)
MRVSSDTNIYEYRALSPQQKAALEMIRADLYKFTVPYENLEYRFYKPDWV
FGLGYQALATVRWKIEPATITVTKKWENVKEGAKKPDVWIQLLKDGKPEG
ERKRIESDKGQTTFEIPNKDEINKYSVKEVDKEGRDWKHKDFTAGQPVNK
GNGHFEITNTKKEKPKIKVTFKKIAGDTNKDLAGAHLVLKKIFDDGNGLL
IKQWDTIGQPVDIDLDAGSYTLTEEKAPDGYMLAAPVSFYVEEDGQIILP
KGEDLEAQNDKTITMVDEKIKEKPTKPSGKLATTVEVDGTKADAQKELEL
SVATDKVTKTVKDTVVYENLLAGETYKLTGQLMKITADKEEEVATKETTF
VADASGNGTTSLEFEDVSLEAGVKYVVYETAESEKEIDEKEGKEKHKVEH
KDKDDKAQTVVVTKEKPTKPSGKLATTVEVDGTKADAQKELELSVATDKV
TKTVKDTVVYENLLAGETYKLTGQLMKITADKEEEVATKETTFVADASGN
GTTSLEFEDVSLEAGVKYVVYETAESEKEIDFKEGKEKHKVEHKDKDDKA
QTVVVTKEKPTKPSGKLATTVEVDGTKADAQKELELSVATDKVTKTVKDT
VVYENLLAGETYKLTGQLMKITADKEEEVATKETTFVADASGNGTTSLEF
EDVSLEAGVKYVVYETAESEKEIDFKEGKEKHKVEHKDKDDKAQTVVVTK
EKPTKPSGKLATTVEVDGTKADAQKELELSVATDKVTKTVKDTVVYENLL
AGETYKLTGQLMKITADKEEEVATKETTFVADASGNGTTSLEFEDVSLEA
GVKYVVYETAESEKEIDFKEGKEKHKVEHKDKDDKAQTVVVSKIKPEPGA
IQEVHFSKVNVGGEEAGAEIHIKQGDTVVASWVSEAGKTHTLKLKPGHYI
FHEAVAPGGYLAVTDIHFSVDETGQVTVTDVNGNTAVAEGNKLTVTDQTK
PVTPPSPEEPGAQEVHFSKVNVGGEEIAGAEIHIKQGDTVVASWVSEAGK
THTLKLKPGHYIFHEAVAPGGYLAVTDIHFSVDETGQVTVTDVNGNTAVA
EGNKLTVTDQTKPVTPPSPEEPGAQEVHFSKVNVGGEEIAGAEIHIKQGD
TVVASWVSEAGKTHTLKLKPGHYIFHEAVAPGGYLAVTDIHFSVDETGQV
TVTDVNGNTAVAEGNKLTVTDQTKPVTPPSPEEPGAQEVHFSKVNVGGEE
IAGAEIHIKQGDTVVASWVSEAGKTHTLKLKPGHYIFHEAVAPGGYLAVT
DIHFSVDETGQVTVTDVNGNTAVAEGNKLTVTDQSADKDKQDKLPNTGET
TGTYLSILGMITAVFASLLYRSKKK

Anchor-3 contains a sortase substrate motif, LPNTG (SEQ ID NO:10), shown in underscore in SEQ ID NO:9, abOve.

23F, INV200, and OXC141 also encode additional polypeptide sequences that are not encoded by S. pneumoniae strain INV104B. These additional polypeptide sequences are disclosed herein in Example 2 as specific examples of sequences that can be used in the methods described herein and as antigens in immunogenic compositions for the production of antibodies and/or the stimulation of an immune response in a subject.

Other Bacterial Pili Polypeptides and Polypeptides from S. pneumoniae

The methods and compositions described herein can be used with pili polypeptides or other polypeptides from any Gram-positive bacterium including, for example, S. pneumoniae. Known and putative pili proteins have been identified in GAS (e.g., Streptococcus pyogenes) (Mora et al., 2005, Proc. Natl. Acad. Sci. USA, 102:15641-6), GBS (e.g., Streptococcus agalactiae) (Lauer et al., 2005, Science, 309:105; WO 2006/078318), Actinomycetes naeslundii (Yeung et al., 1998, Infect. Immun., 66:1482-91), Corynebacterium diphtheriae (Ton-That et al., 2003, Mol. Microbiol., 50:1429-38; Ton-That and Schneewind, 2004, Trends. Microbiol., 12:228-34), Clostridium perfringens, and Enterococcus faecalis. Examples of Gram-positive bacteria include, without limitation, firmicutes such as those of genera Streptococcus (e.g., S. pneumoniae, S. agalactiae, S. pyogenes, S. suis, S. zooepidemicus, S. viridans, S. mutans, S. gordonii, S. equi), Bacillus (e.g., B. anthracis, B. cereus, B. subtilis), Listeria (e.g., L. innocua, L. monocytogenes), Staphylococcus (e.g., S. aureus, S. epidermidis, S. caprae, S. saprophyticus, S. lugdunensis, S. schleiferi), Enterococcus (e.g., E. faecalis, E. faecium), Lactobacillus, Lactococcus (e.g., L. lactis), Leuconostoc (e.g., L. mesenteroi des), Pectinatus, Pediococcus, Acetobacterium, Clostridium (e.g., C. botulinum, C. difficile, C. perfringens, C. tetani), Ruminococcus (e.g., R. albus), Heliobacterium, Heliospirillum, and Sporomusa; and actinobacteria such as those of genera Actinomycetes (e.g., A. naeslundii), Corynebacterium (e.g., C. diphtheriae, C. efficiens), Arthrobacter, Bifidobacterium (e.g., B. longum), Frankia, Micrococcus, Micromonospora, Mycobacterium (e.g., M. tuberculosis, M. leprae, M. Bovis, M. africanum, M. microti), Nocardia (e.g., N. asteroides), Propionibacteriun, and Streptomyces (e.g., S. somaliensis, S. avermitilis, S. coelicolor).

Isolated Pili Polypeptides and Other Polypeptides from S. pneumoniae

Isolated S. pneumoniae Polypeptides, can be used in the methods described herein and as antigens in immunogenic compositions for the production of antibodies and/or the stimulation of an immune response in a subject. Examples of useful S. pneumoniae LPXTG cell wall anchor pili polypeptides include LPXTG-1, LPXTG-1A, LPXTG-2, Anchor-1, Anchor-2, and Anchor-3 (i.e., SEQ ID NOs:2, 4, 6, 7, 8, and 9). Examples of useful S. pneumoniae sortase polypeptides include sort-1, sort-2, sort-23F, and sort-OXC141 (i.e., SEQ ID NOs:676, 1123, 1386, and 282). Variants of S. pneumoniae Polypeptides can also be used in the methods described herein and as antigens in immunogenic compositions for the production of antibodies and/or the stimulation of an immune response in a subject. For example, S. pneumoniae Polypeptides containing at least 80% sequence identity, e.g., at least 85%, at least 90%, at least 95%, at least 98%, or at least 99%, with a S. pneumoniae Polypeptide sequence are also useful in the new methods. Furthermore, a S. pneumoniae Polypeptide sequence with up to 50, e.g., 1, 3, 5, 10, 15, 20, 25, 30, or 40 amino acid insertions, deletions, or substitutions, e.g., conservative amino acid substitutions will be useful in the compositions and methods described herein.

The determination of percent identity between two amino acid sequences can be accomplished using the BLAST 2.0 program, which is available to the public at ncbi.nlm.nih.gov/BLAST. Sequence comparison is performed using an ungapped alignment and using the default parameters (BLOSUM 62 matrix, gap existence cost of 11, per residue gap cost of 1, and a lambda ratio of 0.85). The mathematical algorithm used in BLAST programs is described in Altschul et al., 1997, Nucleic Acids Research, 25:3389-3402.

As used herein, “conservative amino acid substitution” means a substitution of an amino acid in a polypeptide within an amino acid family. Families of amino acids are recognized in the art and are based on physical and chemical properties of the amino acid side chains. Families include the following: amino acids with basic side chains (e.g. lysine, arginine, and histidine); amino acids with acidic side chains (e.g., aspartic acid and glutamic acid); amino acids with uncharged polar side chains (e.g. glycine, asparagine, glutamine, serine, threonine, tyrosine, and cysteine); amino acids with nonpolar side chains (e.g. alanine, valine, leucine, isoleucine, proline, phenylalanine, methionine, and tryptophan); amino acids with branched side chains (e.g., threonine, valine, and isoleucine); and amino acids with aromatic side chains (e.g., tyrosine, phenylalanine, tryptophan, and histidine). An amino acid can belong to more than one family.

Fragments of S. pneumoniae Polypeptides, e.g., immunogenic fragments, are also useful in the methods and compositions described herein. Typically, the fragments are at least 8, 10, 15, 20, 50, 100, 200, or 500 contiguous amino acid residues of a S. pneumoniae Polypeptide. Examples of S. pneumoniae LPXTG polypeptides with useful fragments include LPXTG-1, LPXTG-1A, LPXTG-2, Anchor-1, Anchor-2, or Anchor-3 (e.g., SEQ ID NO:2, 4, 6, 7, 8, or 9). Non-limiting examples of S. pneumoniae sortase polypeptides with useful fragments include sort-1, sort-2, sort-23F, and sort-OXC141 (i.e., SEQ ID NOs:676, 1123, 1386, and 282). In some embodiments the fragments retain at least one biological activity of the full-length protein, such as covalent attachment to a peptidoglycan cell wall or the ability to cross-link to another fragment or protein through an LPXTG motif.

In some embodiments the immunogenic compositions described herein comprise one or more S. pneumoniae Pilus Polypeptides that may be formulated or purified in an oligomeric (pilus) form. In some embodiments, the oligomeric form is a hyperoligomer. In some embodiments the immunogenic compositions described herein comprise one or more S. pneumoniae Pilus Polypeptides that have been isolated in an oligomeric (pilus) form. The oligomer or hyperoligomer pilus structures comprising S. pneumoniae Pilus Polypeptides may be purified or otherwise formulated for use in immunogenic compositions.

One or more of the S. pneumoniae Polypeptides from S. pneumoniae open reading frame polynucleotide sequences may be replaced by a polynucleotide sequence coding for a fragment of the replaced ORF. In some embodiments, one or more of the S. pneumoniae Polypeptides from S. pneumoniae open reading frames may be replaced by a sequence having sequence homology to the replaced ORF.

One or more of the S. pneumoniae Pilus Polypeptide sequences typically include an LPXTG motif (such as LPXTG (SEQ ID NO:10)) or other sortase substrate motif. The LPXTG sortase substrate motif of a S. pneumoniae pilus protein may be generally represented by the formula X₁X₂X₃X₄G (SEQ ID NO:1746), wherein X₁ is an L, a V, an E, a Y, an I, or a Q; wherein X₂ is a P if Xᵢ is an L; wherein X₂ is a V if X₁ is a E or a Q; wherein X₂ is a V or a P if X₁ is a V; wherein X₃ is any amino acid residue; wherein X₄ is a T if X₁ is a V, E, or Q; and wherein X₄ is a T, S, or A if X₁ is an L. Non-limiting examples of LPXTG motifs include YPXTG (SEQ ID NO:11), IPXTG (SEQ ID NO:12), LPXSG (SEQ ID NO:13), VVXTG (SEQ ID NO:14), EVXTG (SEQ ID NO:15), VPXTG (SEQ ID NO:16), QVXTG (SEQ ID NO:17), LPXAG (SEQ ID NO:18), QVPTG (SEQ ID NO:19), FPXTG (SEQ ID NO:20) and VTXTG (SEQ ID NO:21).

The S. pneumoniae Pilus Polypeptides described herein can effect the ability of the S. pneumoniae bacteria to adhere to and invade epithelial cells. These pilus polypeptides may also affect the ability of S. pneumoniae to translocate through an epithelial cell layer. In some embodiments, one or more S. pneumoniae Pilus Polypeptides from S. pneumoniae are capable of binding to or otherwise associating with an epithelial cell surface or synthetic model thereof. In some embodiments one or more S. pneumoniae Pilus Polypeptides bind to or associate with one or more of fibrinogen, fibronectin, or collagen.

The S. pneumoniae sortase proteins are predicted to be involved in the secretion and anchoring of the LPXTG containing surface proteins. The S. pneumoniae pilus II island (INV104B) sortase proteins disclosed herein are encoded by genes (sort-1 and sort-2) found in the same 6.5 kb insert between genes corresponding to sp1008 and sp1009 of TIGR4 as the LPXTG-1 and LPXTG-2 genes discussed above. 23F and OXC141 also encode sortases: sort-23F (SEQ ID NO:1386) and sort-OXC141 (SEQ ID NO:282). Sortase proteins and variants of sortase proteins useful in the methods described herein can be obtained from Gram-positive bacteria.

The S. pneumoniae Pilus Polypeptides can be covalently attached to the bacterial cell wall by membrane-associated transpeptidases, such as a sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In some embodiments, the compositions described herein comprise oligomeric, pilus-like structures comprising S. pneumoniae Pilus Polypeptides such as LPXTG-1, LPXTG-1A, LPXTG-2, Anchor-1, Anchor-2, or Anchor-3 (e.g., SEQ ID NO:2, 4, 6, 7, 8, or 9). The oligomeric, pilus-like structure may comprise numerous units of pilus poplypeptides. In some embodiments, the oligomeric, pilus-like structures comprise two or more pilus proteins. In some embodiments, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises a pilus protein or a fragment thereof. The oligomeric subunits may be covalently associated via a conserved lysine within a pilin motif. The oligomeric subunits may be covalently associated via an LPXTG motif, in some embodiments, via a threonine or serine amino acid residue.

S. pneumoniae Pilus Polypeptides or fragments thereof can be incorporated into the oligomeric, pilus-like structures described herein and will, in some embodiments, include a pilin motif. Oligomeric, pilus-like structures may be used alone or in combination. In some embodiments, the compositions described herein comprise a S. pneumoniae pilus II island (INV104B) pilus in oligomeric form. Further, in some embodiments the S. pneumoniae pilus II island (INV104B) pilus can be organized in a hyperoligomeric form.

### Methods of Purification of Pili

Pili encoded by Streptococcus pneumoniae can be purified from cells, such as bacterial cells, that express Streptococcus pneumoniae pili or pili-like structures by separating the pili from the cells, e.g., by mechanical shearing or enzymatic digestion, and isolating the separated pili.

Suitable bacterial cells for purification of pili include piliated Gram-positive bacterial strains that express Streptococcus pneumoniae Pili Polypeptides, non-piliated Gram-positive bacteria that have been transformed with one or more Gram-positive pilus proteins, such as S. pneumoniae LPXTG-1, LPXTG-1A, LPXTG-2, Anchor-1, Anchor-2, and Anchor-3 (e.g., SEQ ID NOs:2, 4, 6, 7, 8, and 9), and Gram-negative or other cells transformed with one or more Gram-positive pilus proteins, such as S. pneumoniae LPXTG-1, LPXTG-1A, LPXTG-2, Anchor-1, Anchor-2, and Anchor-3 (e.g., SEQ ID NOs:2, 4, 6, 7, S, and 9). Typically, a cell used for purification of pili will produce only the type or types of pili desired, e.g., endogenous or heterologous pili. For the production of heterologous pili, the cell can be altered, e.g., by mutation or recombinant DNA methods, to not produce endogenous pili. Typically, a pili-producing Gram-positive bacterial cell useful for purification will express one or more compatible sortases such that the pili are expressed on the cell surface. Examples of S. pneumoniae pilus II island (INV104B), 23F, INV200, and OXC141 LPXTG cell wall anchor polypeptides that could be purified include LPXTG-1, LPXTG-1A, LPXTG-2, Anchor-1, Anchor-2, and Anchor-3 (e.g., SEQ ID NOs:2, 4, 6, 7, 8, and 9). Examples of S. pneumoniae pilus II island (INV104B), 23F, INV200, and OXC141 sortases that could be purified include sort-1, sort-2, sort-23F, and sort-OXC141 (e.g., SEQ ID NOs:676, 1123, 1386, and 282).

Separation of pili from Gram-positive bacterial cells is typically accomplished by mechanical shearing, enzymatic digestion, decreasing or inhibiting sortase activity, or treatment with a compound that interferes with cell wall integrity. Mechanical shearing can physically remove the pili from the cells, whereas other methods can eliminate the point of attachment of the pili (e.g., by degradation of cell wall or pilus components). Following separation of the pili from the cells, the pili and cells can be separated, e.g., by centrifugation.

Non-limiting examples of mechanical shearing methods include ultrasonication, glass bead shearing, and mixing. Methods of sonication are discussed, for example, in Yamaguchi et al., 2004, Current Microbiol., 49:59-65. Methods of glass bead shearing are discussed, for example, in Levesque et al., 2001, J. Bacteriol., 183:2724-32. General methods of mechanical shearing are discussed, for example, in Wolfgang et al., 1998, Mol. Microbiol., 29:321-30; Trachtenberg et al., 2005, J. Mol. Biol., 346:665-676; Parge et al., 1990, J. Biol. Chem., 265:2278-85; Isaacson et al., 1981, J. Bacteriol., 146:784-9; Korhonen et al., 1980, Infect. Immun., 27:569-75; Hahn et al., 2002, J. Mol. Biol., 323:845-57; St. Geme et al., 1996, Proc. Natl. Acad. Sci. USA, 93:11913-18; Weber et al., 2005, J. Bacteriol., 187:2458-68; and Mu et al., 2002, J. Bacteriol., 184:4868-74.

Non-limiting examples of enzymes suitable for enzymatic digestion include cell-wall degrading enzymes such as mutanolysin, lysostaphin, and lysozymes. Methods of enzymatic digestion are discussed, for example, in Bender et al., 2003, J. Bacteriol., 185:6057-66; Ton-That et al., 2004, Mol. Microbiol., 53:251-61; and Ton-That et al., 2003, Mol. Microbiol., 50:1429-38. For downstream administration to subjects, one can use multiple enzymes to remove cell-wall components that may cause an undesired host reaction.

Non-limiting examples of methods of inhibiting or decreasing sortase activity include decreasing, for example, SrtA activity by introduction of a loss-of-function allele of SrtA, deleting the endogenous SrtA gene, expression of a nucleic acid that decreases SrtA expression (e.g., an antisense or miRNA), and treating the cells with a compound that inhibits SrtA activity (see, e.g., Marrafini et al., Microbiol. Mol. Biol. Rev., 70:192-221, 2006).

Exemplary sortase A inhibitors include methane-thiosulfonates (e.g., MTSET and (2-sulfonatoethyl) methane-thiosulfonate) (Ton-That and Schneewind, J. Biol. Chem., 274:24316-24320, 1999), p-hydroxymercuribenzoic acid, glucosylstero β-sitosterol-3-O-glucopyranol (Kim et al., Biosci. Biotechnol. Biochem., 67:2477-79, 2003), berberine chloride (Kim et al., Biosci. Biotechnol. Biochem., 68:421-24, 2004), peptidyl-diazomethane (LPAT-CHN2) (Scott et al., Biochem. J., 366:953-58, 2002), peptidyl-chloromethane (LPAT-CH2Cl), peptidyl-vinyl sulfone [LPAT-SO₂(Ph)] (Conolly et al., J. Biol. Chem., 278:34061-65, 2003), vinyl sulfones (e.g., di-, ethyl-, methyl-, and phenyl vinyl sulfones) (Frankel et al., J. Am. Chem. Soc., 126:3404-3405, 2004), LPXTG motif peptides with the threonine residue replaced by a phosphinate group (e.g., LPEΨ{PO₂H—CH₂}G) (Kruger et al., Bioorg. Med. Chem., 12:3723-29, 2004), substituted (Z)-diaryl-acrylonitriles (Oh et al., J. Med. Chem., 47:2418-21, 2004), and extracts of various medicinal plants (Kim et al., Biosci. Biotechnol. Biochem., 66:2751-54, 2002).

Non-limiting examples of compounds that interfere with cell wall integrity include glycine and antibiotics such as penicillins (e.g., methicillin, amoxicillin, ampicillin), cephalosporins (e.g., cefalexin, cefproxil, cefepime), glycopeptides (e.g., vancomycin, teicoplanin, ramoplanin), and cycloserine.

Separated pili can be separated from other components by density, for example by using density gradient centrifugation. For example, the pili can be separated by centrifugation on a sucrose gradient.

Typically, a sample containing Gram-positive pili will contain pili oligomers of different molecular weights due to differing numbers of pilus protein subunits present in the pili. To reduce polydispersity, a sample containing Gram-positive pili can be separated by size. For example, a gel filtration or size exclusion column can be used. An ultrafiltration membrane can also be used to reduce polydispersity.

Gram-positive pili can also be isolated using affinity methods such as affinity chromatography. For example, a protein that binds specifically to a Gram-positive pilus, e.g., an antibody that binds specifically to a pilus component or an antibody that binds preferentially to pili, can be immobilized on a solid substrate (e.g., a chromatography substrate), then a sample containing Gram-positive pili can be exposed to the immobilized binding protein. Such affinity isolation methods can also be used to isolate, purify, or enrich preparations of cells that express Gram-positive pili.

Gram-positive pili can also be isolated using any other protein purification method known in the art, e.g., precipitations, column chromatography methods, and sample concentrations. Additional methods are described, e.g., in Ruffolo et al., 1997, Infect. Immun., 65:339-43. Methods of protein purification are described in detail in, e.g., Scopes, R. K., Protein Purification: Principles and Practice, 3rd. ed., 1994, Springer, N.Y.

The presence of Gram-positive pili in fractions during purification can be followed by electrophoresis (e.g., polyacrylamide electrophoresis), measuring binding of an agent that specifically binds to the gram positive pili (e.g., an antibody against a pilus protein or an antibody that binds preferentially to pili), and/or measuring an activity of the pili such as protein or cell binding.

### Antibodies

The S. pneumoniae Polypeptides described herein may also be used to prepare antibodies specific to those S. pneumoniae Polypeptides. In some embodiments the antibodies are specific to an oligomeric or hyper-oligomeric form of a S. pneumoniae Pilus Polypeptide. The compositions described herein also include combinations of antibodies specific to S. pneumoniae pilus polypeptides and other polypeptides from S. pneumoniae selected to provide protection against an increased range of serotypes and strain isolates. For example, such a combination may comprise a first and second antibody, wherein the first antibody is specific to a first S. pneumoniae Polypeptide and the second antibody is specific to a second S. pneumoniae Polypeptide or non-S. pneumoniae Polypeptide.

The specific S. pneumoniae Polypeptide antibodies described herein include one or more biological moieties that, through chemical or physical means, can bind to or associate with an epitope of a S. pneumoniae Polypeptide. The antibodies described herein include antibodies which specifically bind to a S. pneumoniae Polypeptide. The compositions described herein include antibodies obtained from both polyclonal and monoclonal preparations, as well as the following: hybrid (chimeric) antibody molecules (see, e.g., Winter et al. (1991) Nature 349: 293-299; and U.S. Pat. No. 4,816,567; F(ab′)2 and F(ab) fragments; Fv molecules (non-covalent heterodimers, see, e.g., Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5897-5883); dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain immunological binding properties of the parent antibody molecule. The compositions described herein further include antibodies obtained through non-conventional processes, such as phage display.

The antibodies described herein can be polyclonal, monoclonal, recombinant, e.g., chimeric or humanized, fully human, non-human, e.g., murine, or single chain antibodies. Methods of making such antibodies are known. In some cases, the antibodies have effector function and can fix complement. The antibodies can also be coupled to toxins, reporter groups, or imaging agents.

In some embodiments, the specific S. pneumoniae antibodies described herein are monoclonal antibodies. These monoclonal antibodies include an antibody composition having a homogeneous antibody population. The monoclonal antibodies described herein may be obtained from murine hybridomas, as well as human monoclonal antibodies obtained using human rather than murine hybridomas. See, e.g., Cote, et al. Monoclonal Antibodies and Cancer Therapy, Alan R. Liss, 1985, p 77.

Chimeric, humanized, e.g., completely human, antibodies are desirable for applications that include repeated administration, e.g., therapeutic treatment (and some diagnostic applications) of a human subject.

The antibodies described herein can also be used in the prophylactic or therapeutic treatment of S. pneumoniae infection. The antibodies may block the attachment or some other activity of S. pneumoniae on host cells. Additionally, the antibodies can be used to deliver a toxin or therapeutic agent such as an antibiotic to S. pneumoniae cells.

The antibodies described herein may be used in diagnostic applications, for example, to detect the presence or absence of S. pneumoniae Polypeptides in a biological sample. Anti-pili, pilus polypeptide or other polypeptide from S. pneumoniae antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, contrast agents, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerytlirin; examples of contrast agents include electron dense materials useful for electron microscopy, such as gold particles, or magnetically active materials useful for magnetic resonance imaging, such as supermagnetic iron particles; an example of a luminescent material includes luminol; examples of bioluminescent materials include luciferase, luciferin, and aequorin, and examples of suitable radioactive material include ¹²⁵I, ¹³¹I, ³⁵S or ³H. Such diagnostic antibodies can be used in methods to detect the presence of piliated S. pneumoniae containing Streptococcus pneumoniae pilus II island (INV104B) pili in an infected patient, e.g., by testing a sample from the patient. The course of treatment can then be selected based on the presence or absence of Streptococcus pneumoniae Polypeptides. For example, a patient infected with non-piliated S. pneumoniae could be treated with an antibiotic, whereas a patient infected with piliated S. pneumoniae containing S. pneumoniae Pili Polypeptides could also be treated with a pili-binding compound, such as an antibody, and/or an anti-inflammatory agent (e.g., IL-6 or an anti-TNF agent such as an anti-TNF antibody).

### Screening Assays

In some aspects, the methods described herein (also referred to herein as “screening assays”) can be used to identify modulators, i.e., candidate compounds or agents identified from one or more test compounds (e.g., antibodies, proteins, peptides, peptidomimetics, peptoids, small inorganic molecules, small non-nucleic acid organic molecules, nucleic acids (e.g., antisense nucleic acids, siRNA, oligonucleotides, or synthetic oligonucleotides), or other drugs) that inhibit an activity, e.g., a binding activity of S. pneumoniae Pili Polypeptides. Compounds thus identified can be used to modulate the binding activity of S. pneumoniae containing S. pneumoniae Pili Polypeptides or the attachment of such S. pneumoniae in a therapeutic protocol, to elaborate the biological function S. pneumoniae.

In some embodiments, assays are provided for screening test compounds to identify those that can bind to S. pneumoniae Pili Polypeptides or a portion thereof. Compounds that bind to S. pneumoniae Pili Polypeptides can be tested for their ability to modulate an activity associated with S. pneumoniae pili such as attachment, infection, or an inflammatory response.

The test compounds used in the methods described herein can be obtained using any of the numerous approaches in combinatorial library methods known in the art, including: biological libraries; peptoid libraries (libraries of molecules having the functionalities of peptides, but with a novel, non-peptide backbone, which are resistant to enzymatic degradation, but which, nevertheless, remain bioactive; see, e.g., Zuckermann et al., 1994, J. Med. Chem., 37:2678-2685); spatially addressable parallel solid phase or solution phase libraries; synthetic library methods requiring deconvolution; the “one-bead one-compound” library method; and synthetic library methods using affinity chromatography selection. The biological library and peptoid library approaches are limited to peptide libraries, while the other four approaches are applicable to peptide, non-peptide oligomer, or small molecule libraries of compounds (Lam, 1997, Anticancer Drug Des., 12:145).

Examples of methods for the synthesis of molecular libraries can be found in the art, for example in: DeWitt et al. (1993, Proc. Natl. Acad. Sci. USA, 90:6909; Erb et al., 1994, Proc. Natl. Acad. Sci. USA, 91:11422; Zuckermann et al., 1994, J. Med. Chem., 37:2678; Cho et al., 1993, Science, 261:1303; Carrell et al., 1994, Angew. Chem. Int. Ed. Engl., 33:2059; Carell et al., 1994, Angew. Chem. Int. Ed. Engl., 33:2061; and in Gallop et al., 1994, J. Med. Chem., 37:1233).

Libraries of compounds may be presented in solution (e.g., Houghten, 1992, Biotechniques, 13:412-421), or on beads (Lam, 1991, Nature, 354:82-84), chips (Fodor, 1993, Nature, 364:555-556), bacteria (Ladner, U.S. Pat. No. 5,223,409), spores (Ladner, U.S. Pat. No. 5,223,409), plasmids (Cull et al., 1992, Proc. Natl. Acad. Sci. USA, 89:1865-1869), or on phage (Scott and Smith, 1990, Science, 249:386-390; Devlin, 1990, Science, 249:404-406; Cwirla et al., 1990, Proc. Natl. Acad. Sci. USA, 87:6378-6382; Felici, 1991, J. Mol. Biol., 222:301-310; and Ladner supra).

In some embodiments, the assay is a cell-based assay in which a cell, e.g., a bacterial cell, that expresses a S. pneumoniae Polypeptides or biologically active portion thereof is contacted with a test compound, and the ability of the test compound to modulate S. pneumoniae activity is determined, for example, by monitoring cell binding. The cell, for example, can be of mammalian origin, e.g., murine, rat, or human origin. The cell can be an epithelial cell, e.g., a A549 lung epithelial cell.

The ability of the test compound to modulate S. pneumoniae Pili Polypeptides from binding to a ligand or substrate, e.g., a cell or a protein such as fibrinogen, fibronectin, or collagen can be evaluated, for example, by coupling the compound, e.g., the substrate, with a radioisotope or enzymatic label such that binding of the compound, e.g., the substrate, to S. pneumoniae pilus II island (INV104B) pili or pilus protein can be determined by detecting the labeled compound, e.g., substrate, in a complex. Alternatively, S. pneumoniae Polypeptides can be coupled with a radioisotope or enzymatic label to monitor the ability of a test compound to modulate S. pneumoniae Polypeptide binding to a substrate in a complex. For example, compounds (e.g., S. pneumoniae Polypeptide binding partners) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound to interact with a S. pneumoniae Polypeptide with or without the labeling of any of the interactants can be evaluated. For example, a microphysiometer can be used to detect the interaction of a compound with a S. pneumoniae Polypeptide without labeling either the compound or the S. pneumoniae Polypeptide (McConnell et al., 1992, Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and a S. pneumoniae Polypeptide.

In some embodiments, a cell-free assay is provided in which a S. pneumoniae Polypeptide or biologically active portion thereof is contacted with a test compound and the ability of the test compound to bind to the S. pneumoniae Polypeptide or biologically active portion thereof is evaluated. In general, biologically active portions of a S. pneumoniae Polypeptide to be used in the new assays include fragments that participate in interactions with other S. pneumoniae Polypeptide molecules.

Cell-free assays involve preparing a reaction mixture of the target gene protein and the test compound under conditions and for a time sufficient to allow the two components to interact and bind, thus forming a complex that can be removed and/or detected.

An interaction between two molecules can be detected using fluorescence energy transfer (FET) (see, e.g., Lakowicz et al., U.S. Pat. No. 5,631,169 and Stavrianopoulos et al., U.S. Pat. No. 4,868,103). A fluorophore label on the first ‘donor’ molecule is selected such that its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor.’ Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In some embodiments, determining the ability of a S. pneumoniae Polypeptide to bind to a target molecule (e.g., a fibrinogen, fibronectin, or collagen polypeptide or fragment thereof) can be accomplished using real-time Biomolecular Interaction Analysis (BIA) (e.g., Sjolander et al., 1991, Anal. Chem., 63:2338-2345 and Szabo et al., 1995, Curr. Opin. Struct. Biol., 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In some embodiments, the target gene product or the test substance is anchored onto a solid phase. The target gene product/test compound complexes anchored on the solid phase can be detected at the end of the reaction. The target gene product can be anchored onto a solid surface, and the test compound, which is not anchored, can be labeled, either directly or indirectly, with a detectable labelsdiscussed herein.

Multiple target gene products can be anchored onto a solid phase using protein microarray technology, which is also known by other names including: protein chip technology and solid-phase protein array technology. Protein microarray technology is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485):1760-1763, 2000. Microarray substrates include but are not limited to glass, silica, aluminosilicates, borosilicates, metal oxides such as alumina and nickel oxide, various clays, nitrocellulose, or nylon. The microarray substrates may be coated with a compound to enhance synthesis of a probe (e.g., a peptide) on the substrate. Coupling agents or groups on the substrate can be used to covalently link the first amino acid to the substrate. A variety of coupling agents or groups are known to those of skill in the art. Peptide probes can be synthesized directly on the substrate in a predetermined grid. Alternatively, peptide probes can be spotted on the substrate, and in such cases the substrate may be coated with a compound to enhance binding of the probe to the substrate. In these embodiments, presynthesized probes are applied to the substrate in a precise, predetermined volume and grid pattern, preferably utilizing a computer-controlled robot to apply probe to the substrate in a contact-printing manner or in a non-contact manner such as ink jet or piezo-electric delivery. Probes may be covalently linked to the substrate. In some embodiments, one or more control peptide or protein molecules are attached to the substrate. Control peptide or protein molecules allow determination of factors such as peptide or protein quality and binding characteristics, reagent quality and effectiveness, hybridization success, and analysis thresholds and success.

In some embodiments it is desirable to immobilize a S. pneumoniae Polypeptide, an anti-pilus or pilus protein antibody, or a S. pneumoniae Polypeptide binding protein to facilitate separation of complexed from uncomplexed forms of one or both of the proteins, as well as to accommodate automation of the assay. Binding of a test compound to a S. pneumoniae Polypeptide, or interaction of a S. pneumoniae Polypeptide with a target molecule in the presence or absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, glutathione-S-transferase/pilus II island (INV104B) pilus protein fusion proteins or glutathione-S-transferase/target fusion proteins can be adsorbed onto glutathione Sepharose™ beads (Sigma Chemical, St. Louis, Mo.) or glutathione derivatized microtiter plates, which are then combined with the test compound or the test compound and either the non-adsorbed target protein or S. pneumoniae pilus II island (INV104B) pili or pilus protein, and the mixture incubated under conditions conducive to complex formation (e.g., at physiological conditions for salt and pH). Following incubation, the beads or microtiter plate wells are washed to remove unbound components, the matrix immobilized in the case of beads, complex determined either directly or indirectly, for example, as described above. Alternatively, the complexes can be dissociated from the matrix, and the level of S. pneumoniae Polypeptide binding or activity determined using standard techniques.

Other techniques for immobilizing either a S. pneumoniae Polypeptide or a binding target on matrices include using conjugation of biotin and streptavidin. Biotinylated S. pneumoniae Polypeptides or target molecules can be prepared from biotin-NHS (N-hydroxy-succinimide) using techniques known in the art (e.g., biotinylation kits from Pierce Chemicals, Rockford, Ill.), and immobilized in the wells of streptavidin-coated 96 well plates (Pierce Chemical).

To conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In some embodiments, the assay is performed utilizing antibodies that bind specifically to S. pneumoniae Polypeptides or binding targets, but do not interfere with binding of the S. pneumoniae Polypeptide to its target. Such antibodies can be derivatized to the wells of the plate, and unbound target or S. pneumoniae Polypeptide trapped in the wells by antibody conjugation. Methods for detecting such complexes, in addition to those described above for the GST-immobilized complexes, include immunodetection of complexes using antibodies reactive with the S. pneumoniae Polypeptide or target molecule, as well as enzyme-linked assays which rely on detecting an enzymatic activity associated with the S. pneumoniae Polypeptide or target molecule.

In some embodiments, cell-free assays can be conducted in a liquid phase. In such an assay, the reaction products are separated from unreacted components, by any of a number of standard techniques, including but not limited to: differential centrifugation (for example, Rivas et al., 1993, Trends Biochem. Sci., 18:284-287); chromatography (gel filtration chromatography, ion-exchange chromatography); electrophoresis (e.g., Ausubel et al., eds., 1999, Current Protocols in Molecular Biology, J. Wiley: New York.); and immunoprecipitation (for example, Ausubel et al., eds., 1999, Current Protocols in Molecular Biology, J. Wiley: New York). Such resins and chromatographic techniques are known to those skilled in the art (e.g., Heegaard, 1998, J. Mol. Recognit., 11:141-148 and Hage et al., 1997, J. Chromatogr. B. Biomed. Sci. Appl., 699:499-525). Further, fluorescence energy transfer may also be conveniently utilized, as described herein, to detect binding without further purification of the complex from solution.

In some embodiments, the assay includes contacting a S. pneumoniae Pili Polypeptide, or biologically active portion thereof with a known cell or compound (e.g., a protein) that binds to S. pneumoniae Pili Polypeptide, to form an assay mixture, contacting the assay mixture with a test compound, and determining the ability of the test compound to affect binding of the S. pneumoniae Pili Polypeptide to the cell or compound.

A general assay for binding of bacterial cells that express S. pneumoniae pilus II island (INV104B) pili involves incubating bacterial cells that express S. pneumoniae pilus II island (INV104B) pili with A549 lung epithelial cells, washing to remove nonadherent bacterial cells, and detecting adherent bacterial cells. Bacterial adherence can be measured by any means in the art, e.g., detecting binding of an antibody to the adherent bacterial cells or lysing the epithelial cells and counting the number of associated bacterial cells. HEP2 cells, CHO cells, or HeLa cells can also be used in assays of binding of bacterial cells that express S. pneumoniae pilus II island (INV104B) pili.

### Immunogenic Compositions

Immunogenic compositions described herein that include S. pneumoniae Polypeptides may further comprise one or more antigenic agents. Exemplary antigens include those listed below. Additionally, the compositions described herein may be used to treat or prevent infections caused by any of the below-listed microbes. Antigens for use in the immunogenic compositions include, but are not limited to, one or more of the following set forth below, or antigens derived from one or more of the following set forth below:

### Bacterial Antigens

N. meningitidis: a protein antigen from N. meningitides serogroup A, C, W135, Y, and/or B (Bruyn, G. A. W. & van Furth, R. (1991) Eur. J. Clin. Microbiol. Infect. Dis. 10, 897-910; Ryan, M. W. & Antonelli, P. J. (2000) Laryngoscope 110, 961-964; Cutts, F. T., Zaman, S. M., Enwere, G., Jaffar, S., Levine, O, S., Okoko, C. Oluwalana, A., Vaughan, S., Obaro, A., Leach, A., et al. (2005) Lancet 365, 1139-1146; Swiatlo, E., Champlin, F. R., Holman, S. C., Wilson, W. W.&Watt, J. M. (2002) Infect. Immun. 70, 412-415; Sandgren, A., Albiger, B., Orihuela, C., Tuomanen, E., Normark, S. & Henriques-Normark, B. (2005) J. Infect. Dis. 192, 791-800; Henriques Normark, B., Christensson, B., Sandgren, A., Noreen, B., Sylvan, S., Burman, L. G. & Olsson-Liljequist, B. (2003) Microb. Drug Resist. 9, 337-344; Nunes, S., Sá-, Leão, R., Carriço, J., Alves, C. R., Mato, R., Avô, A. B., Saldanha, J., Almeida, J. S., Sanches, I. S. & de Lencastre, H. (2005) J. Clin. Microbiol. 43, 1285-1293); an outer-membrane vesicle (OMV) preparation from N. meningitides serogroup B. (Henrichsen, J. (1995) J. Clin. Microbiol. 33, 2759-2762; Lau, G. W., Haataja, S., Lonetto, M., Kensit, S. E., Marra, A., Bryant, A. P., McDevitt, D., Morrison, D. A. & Holden, D. W. (2001) Mol. Microbiol. 40, 555-571; Rosenow, C., Ryan, P., Weiser, J. N., Johnson, S., Fontan, P., Ortqvist, A. & Masure, H. R. (1997) Mol. Microbiol. 25, 819-829; Tuomanen, E. (1999) Current Opin. Biol. 2, 35-39); a saccharide antigen, including LPS, from N. meningitides serogroup A, B, C W135 and/or Y, such as the oligosaccharide from serogroup C (see PCT/US99/09346; PCT IB98/01665; and PCT IB99/00103);

Streptococcus pneumoniae: a saccharide or protein antigen, particularly a saccharide from Streptococcus pneumoniae or a protein or antigenic peptide of PhtD (BVH-11-2, SP1003, spr0907) (Adamou et al., Infect. Immun., 69:949-53, 2001; Hamel et al., Infect. Immun., 72:2659-70, 2004); PhtE (BVH-3, SP1004, spr0908) (Adamou et al., Infect. Immun., 69:949-53, 2001; Hamel et al., Infect. Immun., 72:2659-70, 2004); PhtB (PhpA, BVH-11, SP1174, Spr1060) (Adamou et al., Infect. Immun., 69:949-53, 2001; Zhang et al., Infect. Immun., 69:3827-36, 2001; Hamel et al., Infect. Immun., 72:2659-70, 2004); PhtA (BVH-11-3, SP1175, sprl 061) (Adamou et al., Infect. Immun., 69:949-53, 2001; Wizemann et al., Infect. Immun., 69:1593-98, 2001; Zhang et al., Infect. Immun., 69:3827-36, 2001; Hamel et al., Infect. Immun., 72:2659-70, 2004); NanA (SP1693, spr1536) (Tong et al., Infect. Immun., 73:7775-78, 2005); SP1872 (spr1687) (Brown et al., Infect. Immun., 69:6702-06, 2001); PspC (CbpA, SP2190, spr1995) (Ogunniyi et al., Infect. Immun., 69:5997-6003, 2001); PspA (SP0177, spr0121, spr1274) (Briles et al., Vaccine, 19:S87-S95, 2001); SP0498 (spr0440); LytB (SP0965, spr0867) (Wizemann et al., Infect. Immun., 69:1593-98, 2001); AliB (SP1527, spr1382); PpmA (SP0981, spr0884) (Overweg et al., Infect. Immun., 68:4180-4188, 2000); LytC(SP1573, spr1431) (Wizemann et al., Infect. Immun., 69:1593-98, 2001); PsaA (Briles et al., Vaccine, 19:S87-S95, 2001); PdB (Ogunniyi et al., Infect. Immun., 69:5997-6003, 2001); RPhp (Zhang et al., Infect. Immun., 69:3827-36, 2001); PiuA (Jomaa et al., Vaccine, 24:5133-39, 2006); PiaA (Jomaa et al., Vaccine, 24:5133-39, 2006); 6PGD (Daniely et al., Clin. Exp. Immunol., 144:254-263, 2006); or PppA (Green et al., Infect. Immun., 73:981-89, 2005);

Streptococcus agalactiae: such as, Group B streptococcus antigens;

Streptococcus pyogenes: such as, Group A streptococcus antigens;

Enterococcus faecalis or Enterococcus faecium: such as, a trisaccharide repeat or other Enterococcus derived antigens provided in U.S. Pat. No. 6,756,361;

Helicobacter pylori: including: Cog, Vac, Nap, HopX, HopY and/or urease antigen;

Bordetella pertussis: such as pertussis holotoxin (PT) and filamentous hemagglutinin (FHA) from B. pertussis, optionally also combination with pertactin and/or agglutinogens 2 and 3 antigen;

Staphylococcus aureus: including S. aureus type 5 and 8 capsular polysaccharides optionally conjugated to nontoxic recombinant Pseudomonas aeruginosa exotoxin A, such as StaphVAX™, or antigens derived from surface proteins, invasins (leukocidin, kinases, hyaluronidase), surface factors that inhibit phagocytic engulfment (capsule, Protein A), carotenoids, catalase production, Protein A, coagulase, clotting factor, and/or membrane-damaging toxins (optionally detoxified) that lyse eukaryotic cell membranes (hemolysins, leukotoxin, leukocidin);

Staphylococcus epidermis: particularly, S. epidermidis slime-associated antigen (SAA);

Staphylococcus saprophyticus: (causing urinary tract infections) particularly the 160 kDa hemagglutinin of S. saprophyticus antigen;

Pseudomonas aeruginosa: particularly, endotoxin A, Wzz protein, P. aeruginosa LPS, more particularly LPS isolated from PAO1 (O5 serotype), and/or Outer Membrane Proteins, including Outer Membrane Proteins F (OprF) (Infect Immun. 2001 May; 69(5): 3510-3515);

Bacillus anthracis (anthrax): such as B. anthracis antigens (optionally detoxified) from A-components (lethal factor (LF) and edema factor (EF)), both of which can share a common B-component known as protective antigen (PA);

Moraxella catarrhalis: (respiratory) including outer membrane protein antigens (HMW-OMP), C-antigen, and/or LPS;

Yersinia pestis (plague): such as F1 capsular antigen (Infect Immun. 2003 January; 71(1)): 374-383, LPS (Infect Immun. 1999 October; 67(10): 5395), Yersinia pestis V antigen (Infect Immun. 1997 November; 65(11): 4476-4482);

Yersinia enterocolitica (gastrointestinal pathogen): particularly LPS (Infect Immun. 2002 August; 70(8): 4414);

Yersinia pseudotuberculosis: gastrointestinal pathogen antigens;

Mycobacterium tuberculosis: such as lipoproteins, LPS, BCG antigens, a fusion protein of antigen 85B (Ag85B) and/or ESAT-6 optionally formulated in cationic lipid vesicles (Infect Immun. 2004 October; 72(10): 6148), Mycobacterium tuberculosis (Mtb) isocitrate dehydrogenase associated antigens (Proc. Natl. Acad Sci U.S.A. 2004 Aug. 24; 101(34): 12652), and/or MPT51 antigens (Infect Immun. 2004 July; 72(7): 3829);

Legionella pneumophila (Legionnaires' Disease): L. pneumophila antigens—optionally derived from cell lines with disrupted asd genes (Infect Immun. 1998 May; 66(5): 1898);

Rickettsia: including outer membrane proteins, including the outer membrane protein A and/or B (OmpB) (Biochem Biophys Acta. 2004 Nov. 1; 1702(2):145), LPS, and surface protein antigen (SPA) (J Autoimmun. 1989 June; 2 Suppl:81);

E. coli: including antigens from enterotoxigenic E. coli (ETEC), enteroaggregative E. coli (EAggEC), diffusely adhering E. coli (DAEC), enteropathogenic E. coli (EPEC), and/or enterohemorrhagic E. coli (EHEC);

Vibrio cholerae: including proteinase antigens, LPS, particularly lipopolysaccharides of Vibrio cholerae II, O1 Inaba O-specific polysaccharides, V. cholera O139, antigens of IEM108 vaccine (Infect Immun. 2003 October; 71(10):5498-504), and/or Zonula occludens toxin (Zot);

Salmonella typhi (typhoid fever): including capsular polysaccharides preferably conjugates (Vi, i.e. vax-TyVi);

Salmonella typhimurium (gastroenteritis): antigens derived therefrom are contemplated for microbial and cancer therapies, including angiogenesis inhibition and modulation of flk;

Listeria monocytogenes (systemic infections in immunocompromised or elderly people, infections of fetus): antigens derived from L. monocytogenes are preferably used as carriers/vectors for intracytoplasmic delivery of the conjugates/associated compositions described herein;

Porphyromonas gingivalis: such as, P. gingivalis outer membrane protein (OMP);

Tetanus: such as tetanus toxoid (TT) antigens, e.g., used as a carrier protein in conjunction/conjugated with the compositions described herein;

Diphtheria: such as a diphtheria toxoid or a diphtheria toxoid mutant, e.g., CRM₁₉₇, additionally antigens capable of modulating, inhibiting or associated with ADP ribosylation are contemplated for combination/co-administration/conjugation with the compositions described herein, the diphtheria toxoids can be used as carrier proteins;

Borrelia burgdorferi (Lyme disease): such as antigens associated with P39 and P13 (an integral membrane protein, Infect Immun. 2001 May; 69(5): 3323-3334), VlsE Antigenic Variation Protein (J Clin Microbiol. 1999 December; 37(12): 3997);

Haemophilus influenzae B: such as a saccharide antigen therefrom;

Klebsiella: such as an OMP, including OMP A, or a polysaccharide optionally conjugated to tetanus toxoid;

Neiserria gonorrhoeae: including, a Por (or porin) protein, such as PorB (see Zhu et al., Vaccine (2004) 22:660-669), a transferring binding protein, such as TbpA and TbpB (see Price et al., Infection and Immunity (2004) 71(1):277-283), a opacity protein (such as Opa), a reduction-modifiable protein (Rmp), and outer membrane vesicle (OMV) preparations (see Plante et al., J Infectious Disease (2000) 182:848-855), also see, e.g., WO99/24578, WO99/36544, WO99/57280, WO02/079243);

Chlamydia pneumoniae: particularly C. pneumoniae protein antigens;

Chlamydia trachomatis: including antigens derived from serotypes A, B, Ba and C are (agents of trachoma, a cause of blindness), serotypes L₁, L₂ & L₃ (associated with Lymphogranuloma venereum), and serotypes, D-K;

Treponema pallidum (Syphilis): particularly a TmpA antigen; and

Haemophilus ducreyi (causing chancroid): including outer membrane protein (DsrA).

Where not specifically referenced, further bacterial antigens as described herein may be capsular antigens, polysaccharide antigens or protein antigens of any of the above. Further bacterial antigens may also include an outer membrane vesicle (OMV) preparation. Additionally, antigens include live, attenuated, split (for enveloped viruses), and/or purified versions of any of the aforementioned bacteria. The bacterial or microbial derived antigens described herein may be gram-negative or gram-positive and aerobic or anaerobic.

Additionally, any of the above bacterial-derived saccharides (polysaccharides, LPS, LOS or oligosaccharides) can be conjugated to another agent or antigen, such as a carrier protein (for example CRM₁₉₇). Such conjugation may be direct conjugation effected by reductive amination of carbonyl moieties on the saccharide to amino groups on the protein, as provided in U.S. Pat. No. 5,360,897 and Can J Biochem Cell Biol. 1984 May; 62(5):270-5. Alternatively, the saccharides can be conjugated through a linker, such as, with succinamide or other linkages provided in Bioconjugate Techniques, 1996 and CRC, Chemistry of Protein Conjugation and Cross-Linking, 1993.

### Viral Antigens

Influenza: including whole viral particles (attenuated), split, or subunit comprising hemagglutinin (HA) and/or neuraminidase (NA) surface proteins, the influenza antigens may be derived from chicken embryos or propagated on cell culture, and/or the influenza antigens are derived from influenza type A, B, and/or C, among others;

Respiratory syncytial virus (RSV): including the F protein of the A2 strain of RSV (J Gen Virol. 2004 November; 85(Pt 11):3229) and/or G glycoprotein;

Parainfluenza virus (PIV): including PIV type 1, 2, and 3, preferably containing hemagglutinin, neuraminidase and/or fusion glycoproteins;

Poliovirus: including antigens from a family of picornaviridae, preferably poliovirus antigens such as OPV or, preferably IPV;

Measles: including split measles virus (MV) antigen optionally combined with the Protollin and or antigens present in MMR vaccine;

Mumps: including antigens present in MMR vaccine;

Rubella: including antigens present in MMR vaccine as well as other antigens from Togaviridae, including dengue virus;

Rabies: such as lyophilized inactivated virus (RabAvert™);

Flaviviridae viruses: such as (and antigens derived therefrom) yellow fever virus, Japanese encephalitis virus, dengue virus (types 1, 2, 3, or 4), tick borne encephalitis virus, and West Nile virus;

Caliciviridae; antigens therefrom;

HIV: including HIV-1 or HIV-2 strain antigens, such as gag (p24gag and p55gag), env (gp160 and gp41), pol, tat, nef, rev vpu, miniproteins, (preferably p55 gag and gp140v delete) and antigens from the isolates HIV_{IIIb}, HIV_{SF2}, HIV_{LAV}, HIV_{LAI}, HIV_{MN}, HIV-1_{CM235}, HIV-1_{US4}, HIV-2; simian immunodeficiency virus (SW) among others;

Rotavirus: including VP4, VP5, VP6, VP7, VP8 proteins (Protein Expr Purif. 2004 December; 38(2):205) and/or NSP4;

Pestivirus: such as antigens from classical porcine fever virus, bovine viral diarrhea virus, and/or border disease virus;

Parvovirus: such as parvovirus B19;

Coronavirus: including SARS virus antigens, particularly spike protein or proteases therefrom, as well as antigens included in WO 04/92360;

Hepatitis A virus: such as inactivated virus;

Hepatitis B virus: such as the surface and/or core antigens (sAg), as well as the presurface sequences, pre-S1 and pre-S2 (formerly called pre-S), as well as combinations of the above, such as sAg/pre-S1, sAg/pre-S2, sAg/pre-S1/pre-S2, and pre-S1/pre-S2, (see, e.g., AHBV Vaccines—Human Vaccines and Vaccination, pp. 159-176; and U.S. Pat. Nos. 4,722,840, 5,098,704, 5,324,513; Beames et al., J. Virol. (1995) 69:6833-6838, Birnbaum et al., J. Virol. (1990) 64:3319-3330; and Zhou et al., J. Virol. (1991) 65:5457-5464);

Hepatitis C virus: such as E1, E2, E1/E2 (see, Houghton et al., Hepatology (1991) 14:381), NS345 polyprotein, NS 345-core polyprotein, core, and/or peptides from the nonstructural regions (International Publication Nos. WO 89/04669; WO 90/11089; and WO 90/14436);

Delta hepatitis virus (HDV): antigens derived therefrom, particularly 8-antigen from HDV (see, e.g., U.S. Pat. No. 5,378,814);

Hepatitis E virus (HE V); antigens derived therefrom;

Hepatitis G virus (HGV); antigens derived therefrom;

Varcicella zoster virus: antigens derived from varicella zoster virus (VZV) (J. Gen. Virol. (1986) 67:1759);

Epstein-Barr virus: antigens derived from EBV (Baer et al., Nature (1984) 310:207);

Cytomegalovirus: CMV antigens, including gB and gH (Cytomegaloviruses (J. K. McDougall, ed., Springer-Verlag 1990) pp. 125-169);

Herpes simplex virus: including antigens from HSV-1 or HSV-2 strains and glycoproteins gB, gD and gH (McGeoch et al., J. Gen. Virol. (1988) 69:1531 and U.S. Pat. No. 5,171,568);

Human Herpes Virus: antigens derived from other human herpesviruses such as HHV6 and HHV7; and

HPV: including antigens associated with or derived from human papillomavirus (HPV), for example, one or more of E1-E7, L1, L2, and fusions thereof, particularly the compositions described herein may include a virus-like particle (VLP) comprising the L1 major capsid protein, more particular still, the HPV antigens are protective against one or more of HPV serotypes 6, 11, 16 and/or 18.

Further provided are antigens, compositions, methods, and microbes included in Vaccines, 4^{th} Edition (Plotkin and Orenstein ed. 2004); Medical Microbiology 4^{th} Edition (Murray et al. ed. 2002); Virology, 3rd Edition (W. K. Joklik ed. 1988); Fundamental Virology, 2nd Edition (B. N. Fields and D. M. Knipe, eds. 1991), which are contemplated in conjunction with the compositions described herein.

Additionally, antigens include live, attenuated, split, and/or purified versions of any of the aforementioned viruses.

### Fungal Antigens

Fungal antigens for use herein that are associated with vaccines include those described in: U.S. Pat. Nos. 4,229,434 and 4,368,191 for prophylaxis and treatment of trichopytosis caused by Trichophyton mentagrophytes; U.S. Pat. Nos. 5,277,904 and 5,284,652 for a broad spectrum dermatophyte vaccine for the prophylaxis of dermatophyte infection in animals, such as guinea pigs, cats, rabbits, horses and lambs, these antigens comprises a suspension of killed T. equinum, T. mentagrophytes (var. granulare), M. canis and/or M. gypseum in an effective amount optionally combined with an adjuvant; U.S. Pat. Nos. 5,453,273 and 6,132,733 for a ringworm vaccine comprising an effective amount of a homogenized, formaldehyde-killed fungi, i.e. Microsporum canis culture in a carrier; and U.S. Pat. No. 5,948,413 involving extracellular and intracellular proteins for pythiosis. Additional antigens identified within antifungal vaccines include Ringvac bovis LTF-130 and Bioveta.

Further, fungal antigens for use herein may be derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme.

Fungal pathogens for use as antigens or in derivation of antigens in conjunction with the compositions described herein comprise Aspergillus fumigatus, Aspergillus flavus, Aspergillus niger, Aspergillus nidulans, Aspergillus terreus, Aspergillus sydowi, Aspergillus flavatus, Aspergillus glaucus, Blastoschizomyces capitatus, Candida albicans, Candida enolase, Candida tropicalis, Candicla glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitiS, Blastomyces dermaticlis, Cryptococcus neoformans, Geotrichum clavatum, Histoplasma capsulation, Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, and Saksenaea spp.

Other fungi from which antigens are derived include Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

Processes for producing fungal antigens are well known in the art (see U.S. Pat. No. 6,333,164). In some methods a solubilized fraction extracted and separated from an insoluble fraction obtainable from fungal cells of which cell wall has been substantially removed or at least partially removed, characterized in that the process comprises: obtaining living fungal cells; obtaining fungal cells of which cell wall has been substantially removed or at least partially removed; bursting the fungal cells of which cell wall has been substantially removed or at least partially removed; obtaining an insoluble fraction; and extracting and separating a solubilized fraction from the insoluble fraction.

### STD Antigens

In some embodiments, microbes (bacteria, viruses and/or fungi) against which the present compositions and methods can be implement include those that cause sexually transmitted diseases (STDs) and/or those that display on their surface an antigen that can be the target or antigen composition as described herein. In some embodiment, compositions are combined with antigens derived from a viral or bacterial STD. Antigens derived from bacteria or viruses can be administered in conjunction with the compositions described herein to provide protection against at least one of the following STDs, among others: chlamydia, genital herpes, hepatitis (particularly HCV), genital warts, gonorrhea, syphilis and/or chancroid (see, WO00/15255).

In some embodiments, the compositions described herein are co-administered with one or more antigens for the prevention or treatment of an STD.

Antigens derived from the following viruses associated with STDs, which are described in greater detail above, are preferred for co-administration with the compositions described herein: hepatitis (particularly HCV), HPV, HIV, or HSV.

Additionally, antigens derived from the following bacteria associated with STDs, which are described in greater detail above, are preferred for co-administration with the compositions described herein: Neiserria gonorrhoeae, Chlamydia pneumoniae, Chlamydia trachomatis, Treponema pallidum, or Haemophilus ducreyi.

### Respiratory Antigens

The S. pneumoniae antigen may be a respiratory antigen and could further be used in an immunogenic composition for methods of preventing and/or treating infection by a respiratory pathogen, including a virus, bacteria, or fungi such as respiratory syncytial virus (RSV), PIV, SARS virus, influenza, Bacillus anthracis, particularly by reducing or preventing infection and/or one or more symptoms of respiratory virus infection. A composition comprising an antigen described herein, such as one derived from a respiratory virus, bacteria or fungus is administered in conjunction with the compositions described herein to an individual which is at risk of being exposed to that particular respiratory microbe, has been exposed to a respiratory microbe or is infected with a respiratory virus, bacteria or fungus. The composition(s) described herein is/are co-administered at the same time or in the same formulation with one or more antigens of the respiratory pathogen. Administration of the composition results in reduced incidence and/or severity of one or more symptoms of respiratory infection.

### Pediatric/Geriatric Antigens

In some embodiments, the compositions described herein are used in conjunction with one or more antigens for treatment of a pediatric population, as in a pediatric antigen. In some embodiments, the age of the subjects in the pediatric population is less than about 3 years old, or less than about 2 years, or less than about 1 years old. In some embodiments the pediatric antigen (in conjunction with the composition described herein) is administered multiple times over at least 1, 2, or 3 years.

In some embodiments, the compositions described herein are used in conjunction with one or more antigens for treatment of a geriatric population, as in a geriatric antigen. In some embodiments, the age of the subjects in the geriatric population is greater than about 50 years old, greater than about 55 years old, greater than about 60 years old, greater than about 65 years old, greater than about 70 years old, greater than about 75 years old, greater than about 80 years old, or greater than about 85 years old. In some embodiments the geriatric antigen (in conjunction with the composition described herein) is administered multiple times over at least 1, 2, or 3 years.

### Other Antigens

Other antigens for use in conjunction with the compositions of the present include hospital acquired (nosocomial) associated antigens.

In some embodiments, parasitic antigens are contemplated in conjunction with the compositions described herein. Examples of parasitic antigens include those derived from organisms causing malaria and/or Lyme disease.

In some embodiments, the antigens in conjunction with the compositions described herein are associated with or effective against a mosquito born illness. In some embodiments, the antigens in conjunction with the compositions described herein are associated with or effective against encephalitis. In some embodiments the antigens in conjunction with the compositions described herein are associated with or effective against an infection of the nervous system.

In some embodiments, the antigens in conjunction with the compositions described herein are antigens transmissible through blood or body fluids.

### Antigen Formulations

In some aspects, methods of producing microparticles having adsorbed antigens are provided. The methods comprise: (a) providing an emulsion by dispersing a mixture comprising (i) water, (ii) a detergent, (iii) an organic solvent, and (iv) a biodegradable polymer selected from the group consisting of a poly(α-hydroxy acid), a polyhydroxy butyric acid, a polycaprolactone, a polyorthoester, a polyanhydride, and a polycyanoacrylate. The polymer is typically present in the mixture at a concentration of about 1% to about 30% relative to the organic solvent, while the detergent is typically present in the mixture at a weight-to-weight detergent-to-polymer ratio of from about 0.00001:1 to about 0.1:1 (more typically about 0.0001:1 to about 0.1:1, about 0.001:1 to about 0.1:1, or about 0.005:1 to about 0.1:1); (b) removing the organic solvent from the emulsion; and (c) adsorbing an antigen on the surface of the microparticles. In some embodiments, the biodegradable polymer is present at a concentration of about 3% to about 10% relative to the organic solvent.

In some embodiments, microparticles for use herein are formed from materials that are sterilizable, non-toxic and biodegradable. Such materials include, without limitation, poly(α-hydroxy acid), polyhydroxybutyric acid, polycaprolactone, polyorthoester, polyanhydride, PACA, and polycyanoacrylate. In some embodiments, microparticles for use with the methods described herein are derived from a poly(α-hydroxy acid), in particular, from a poly(lactide) (“PLA”) or a copolymer of D,L-lactide and glycolide or glycolic acid, such as a poly(D,L-lactide-co-glycolide) (“PLG” or “PLGA”), or a copolymer of D,L-lactide and caprolactone. The microparticles may be derived from any of various polymeric starting materials which have a variety of molecular weights and, in the case of the copolymers such as PLG, a variety of lactide:glycolide ratios, the selection of which will be largely a matter of choice, depending in part on the coadministered macromolecule. These parameters are discussed more fully below.

Further antigens may also include an outer membrane vesicle (OMV) preparation.

Antigens can also be adsorbed to peptidoglycans of various gram-positive bacteria to make gram-positive enhancer matrix (GEM) particles, as described in Bosma et al., Appl. Env. Microbiol., 72:880-889, 2006, the entire contents of which are incorporated herein by reference. This method relies on the non-covalent binding of the LysM motif (Buist et al., J. Bact., 177:1554-63, 1995; Bateman and Bycroft, J. Mol. Biol., 299:1113-19, 2000) to the cell wall peptidoglycan of acid-treated cells. Briefly, a polypeptide antigen linked to one or more LysM motifs (e.g., non-covalently or covalently (e.g., as a fusion protein or by conjugation) is added to acid-treated gram-positive bacteria. The antigen peptides bind with high affinity and can be used in immunogenic compositions. Exemplary acids used in these methods include trichloroacetic acid (e.g., at 0.1%-10%), acetic acid (e.g., at 5.6 M), HCl (e.g., at 0.01 M), lactic acid (e.g., at 0.72 M), and formic acid (e.g., at 0.56 M).

Additional formulation methods and antigens (especially tumor antigens) are provided in U.S. Pat. No. 6,884,435.

### Antigen References

The following references, each of which is specifically incorporated by reference in its entirety, include antigens useful in conjunction with the compositions described herein:
International patent application WO99/24578
International patent application WO99/36544.
International patent application WO99/57280.
International patent application WO00/22430.
Tettelin et al. (2000) Science 287:1809-1815.
International patent application WO96/29412.
Pizza et al. (2000) Science 287:1816-1820.
PCT WO 01/52885.
Bjune et al. (1991) Lancet 338(8775).
Fuskasawa et al. (1999) Vaccine 17:2951-2958.
Rosenqist et al. (1998) Dev. Biol. Strand 92:323-333.
Costantino et al. (1992) Vaccine 10:691-698.
Costantino et al. (1999) Vaccine 17:1251-1263.
Watson (2000) Pediatr Infect Dis J 19:331-332.
Rubin (20000) Pediatr Clin North Am 47:269-285, v.
Jedrzejas (2001) Microbiol Mol Biol Rev 65:187-207.
International patent application filed on 3 Jul. 2001 claiming priority from GB0016363.4; WO 02/02606; PCT IB/01/00166.
Kalman et al. (1999) Nature Genetics 21:385-389.
Read et al. (2000) Nucleic Acids Res 28:1397-406.
Shirai et al. (2000) J. Infect. Dis 181(Suppl 3):S524-S527.
International patent application WO99/27105.
International patent application WO00/27994.
International patent application WO00/37494.
International patent application WO99/28475.
Bell (2000) Pediatr Infect Dis J 19:1187-1188.
Iwarson (1995) APMIS 103:321-326.
Gerlich et al. (1990) Vaccine 8 Suppl:S63-68 & 79-80.
Hsu et al. (1999) Clin Liver Dis 3:901-915.
Gastofsson et al. (1996) N. Engl. J. Med. 334-:349-355.
Rappuoli et al. (1991) TIBTECH 9:232-238.
Vaccines (1988) eds. Plotkin & Mortimer. ISBN 0-7216-1946-0.
Del Guidice et al. (1998) Molecular Aspects of Medicine 19:1-70.
International patent application WO93/018150.
International patent application WO99/53310.
International patent application WO98/04702.
Ross et al. (2001) Vaccine 19:135-142.
Sutter et al. (2000) Pediatr Clin North Am 47:287-308.
Zimmerman & Spann (1999) Am Fan Physician 59:113-118, 125-126.
Dreensen (1997) Vaccine 15 Suppl“S2-6.
MMWR Morb Mortal Wkly rep 1998 January 16:47(1):12, 9.
McMichael (2000) Vaccinel 9 Suppl 1:S101-107.
Schuchat (1999) Lancet 353(9146):51-6.
GB patent applications 0026333.5, 0028727.6 & 0105640.7.
Dale (1999) Infect Disclin North Am 13:227-43, viii.
Ferretti et al. (2001) PNAS USA 98: 4658-4663.
Kuroda et al. (2001) Lancet 357(9264):1225-1240; see also pages 1218-1219.
Ramsay et al. (2001) Lancet 357(9251):195-196.
Lindberg (1999) Vaccine 17 Suppl 2:S28-36.
Buttery & Moxon (2000) J R Coil Physicians Long 34:163-168.
Ahmad & Chapnick (1999) Infect Dis Clin North Am 13:113-133, vii.
Goldblatt (1998) J. Med. Microbiol. 47:663-567.
European patent 0 477 508.
U.S. Pat. No. 5,306,492.
International patent application WO98/42721.
Conjugate Vaccines (eds. Cruse et al.) ISBN 3805549326, particularly vol. 10:48-114.
Hermanson (1996) Bioconjugate Techniques ISBN: 012323368 & 012342335X.
European patent application 0372501.
European patent application 0378881.
European patent application 0427347.
International patent application WO93/17712.
International patent application WO98/58668.
European patent application 0471177.
International patent application WO00/56360.
International patent application WO00/67161.

### Fusion Proteins

The S. pneumoniae polypeptides used with the compositions described herein may be present in the compositions as individual separate polypeptides, but in some embodiments at least two (i.e. 2, 3, 4, 5, 6, 7, 8, 9, 10, 11, 12, 13, 14, 15, 16, 17, or 18) of the antigens can be expressed as a single polypeptide chain (a “hybrid” or “fusion” polypeptide). Such fusion polypeptides offer two principal advantages: first, a polypeptide that may be unstable or poorly expressed on its own can be assisted by adding a suitable fusion partner that overcomes the problem; second, commercial manufacture is simplified as only one expression and purification need be employed in order to produce two polypeptides which are both antigenically useful.

The fusion polypeptide may comprise one or more polypeptide sequences encoded by the S. pneumoniae Polypeptides as identified herein. Accordingly, the compositions described herein may include a fusion peptide comprising a first amino acid sequence and a second amino acid sequence, wherein said first and second amino acid sequences are selected from a protein encoded by the S. pneumoniae pilus II island (INV104B) or a fragment thereof. In some embodiments, the first and second amino acid sequences in the fusion polypeptide comprise different epitopes.

In some embodiments, hybrids (or fusions) consisting of amino acid sequences from two, three, four, five, six, seven, eight, nine, or ten antigens encoded by the S. pneumoniae Polypeptide sequences discussed herein are preferred. In some embodiments, hybrids consisting of amino acid sequences from two, three, four, or five antigens encoded by the S. pneumoniae Polypeptide sequences discussed herein are preferred.

Different hybrid polypeptides may be mixed together in a single formulation. Within such combinations, a sequence encoded by the S. pneumoniae Polypeptide sequences discussed herein may be present in more than one hybrid polypeptide and/or as a non-hybrid polypeptide. In some embodiments, however, that an antigen is present either as a hybrid or as a non-hybrid, but not as both.

Hybrid polypeptides can be represented by the formula NH₂-A-{-X-L-}ₙ-B—COOH, wherein: X is an amino acid sequence of a S. pneumoniae Polypeptide as described herein or a fragment thereof; L is an optional linker amino acid sequence; A is an optional N-terminal amino acid sequence; B is an optional C-terminal amino acid sequence; and n is 2, 3, 4, 5, 6, 7, 8, 9, 10, 11, 12, 13, 14 or 15.

If a —X— moiety has a leader peptide sequence in its wild-type form, this may be included or omitted in the hybrid protein. In some embodiments, the leader peptides will be deleted except for that of the —X— moiety located at the N-terminus of the hybrid protein i.e. the leader peptide of X₁ will be retained, but the leader peptides of X₂ . . . Xₙ will be omitted. This is equivalent to deleting all leader peptides and using the leader peptide of X₁ as moiety -A-.

For each n instances of {-X-L-}, linker amino acid sequence -L- may be present or absent. For instance, when n=2 the hybrid may be NH₂—X₁-L₁-X₂-L₂-COOH; NH₂—X₁—X₂—COOH; NH₂—Xᵢ-L₁-X₂—COOH; NH₂—X₁—X₂-L₂-COOH; etc. Linker amino acid sequence(s)-L-will typically be short (e.g. 20 or fewer amino acids i.e. 19, 18, 17, 16, 15, 14, 13, 12, 11, 10, 9, 8, 7, 6, 5, 4, 3, 2, 1). Examples comprise short peptide sequences that facilitate cloning, poly-glycine linkers (i.e. comprising Glyₙ where n=2, 3, 4, 5, 6, 7, 8, 9, 10 or more). Other suitable linker amino acid sequences will be apparent to those skilled in the art. A useful linker is GSGGGG, with the Gly-Ser dipeptide being formed from a BamHI restriction site, thus aiding cloning and manipulation, and the (Gly)₄ tetrapeptide being a typical poly-glycine linker.

In the hybrid polypeptide formula provided above, -A- is an optional N-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification. Other suitable N-terminal amino acid sequences will be apparent to those skilled in the art. If X₁ lacks its own N-terminus methionine, -A- is preferably an oligopeptide (e.g. with 1, 2, 3, 4, 5, 6, 7 or 8 amino acids) which provides a N-terminus methionine.

In the hybrid polypeptide formula provided above, —B— is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification, or sequences which enhance protein stability. Other suitable C-terminal amino acid sequences will be apparent to those skilled in the art.

In the hybrid polypeptide formula provided above, most preferably, n is 2 or 3.

### Nucleic Acids

This description provides nucleic acids encoding the S. pneumoniae Polypeptide sequences and/or the hybrid fusion polypeptides. This description also provides nucleic acids encoding S. pneumoniae Polypeptide antigen, and/or the hybrid fusion polypeptides described herein. Furthermore, the description provides nucleic acids which can hybridize to these nucleic acids, preferably under “high stringency” conditions (e.g. 65° C. in a 0.1×SSC, 0.5% SDS solution).

The polypeptides described herein can be prepared by various means (e.g. recombinant expression, purification from cell culture, chemical synthesis, etc.) and in various forms (e.g. native, fusions, non-glycosylated, lipidated, etc.). They are preferably prepared in substantially pure form (i.e. substantially free from other host cell proteins).

The nucleic acids described herein can be prepared in many ways (e.g. by chemical synthesis, from genomic or cDNA libraries, from the organism itself, etc.) and can take various forms (e.g. single stranded, double stranded, vectors, probes, etc.). They are preferably prepared in substantially pure form (i.e. substantially free from other host cell nucleic acids).

The terms “nucleic acid” and “nucleic acids,” as used herein, include DNA and RNA, and also their analogs, such as those containing modified backbones (e.g., phosphorothioates, etc.), and also peptide nucleic acids (PNA), etc. The compositions described herein include nucleic acids comprising sequences complementary to those described above (e.g., for antisense or probing purposes).

This description also discloses a process for producing a polypeptide, comprising the step of culturing a host cell transformed with nucleic acid as described herein under conditions which induce polypeptide expression.

This description also discloses a process for producing a polypeptide, comprising the step of synthesizing at least part of the polypeptide by chemical means.

This description also discloses a process for producing nucleic acid, comprising the step of amplifying nucleic acid using a primer-based amplification method (e.g. PCR).

This description also discloses a process for producing nucleic acid, comprising the step of synthesizing at least part of the nucleic acid by chemical means.

### Purification and Recombinant Expression

The S. pneumoniae Polypeptide sequences described herein may be isolated from native S. pneumoniae, or they may be recombinantly produced, for instance in a heterologous host. For example, the S. pneumoniae pilus II island (INV104B) antigens described herein may be isolated from S. pneumoniae containing the S. pneumoniae pilus II island (INV104B), or they may be recombinantly produced, for instance, in a heterologous host.

The heterologous host may be prokaryotic (e.g., a bacterium) or eukaryotic. It is preferably E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), S. gordonii, L. lactis, yeasts, etc.

Recombinant production of polypeptides may be facilitated by adding a tag protein to the S. pneumoniae Polypeptide sequences as described herein to be expressed as a fusion protein comprising the tag protein and the polypeptide. For example, recombinant production of polypeptides is facilitated by adding a tag protein to the S. pneumoniae pilus II island (INV104B) pilus antigen to be expressed as a fusion protein comprising the tag protein and the S. pneumoniae pilus II island (INV104B) pilus antigen. Such tag proteins can facilitate purification, detection and stability of the expressed protein. Tag proteins suitable for use with the compositions described herein include a polyarginine tag (Arg-tag), FLAG-tag, Strep-tag, c-myc-tag, S-tag, calmodulin-binding peptide, cellulose-binding domain, SBP-tag-chitin-binding domain, glutathione S-transferase-tag (GST), maltose-binding protein, transcription termination anti-termination factor (NusA), E. coli thioredoxin (TrxA) and protein disulfide isomerase I (DsbA). Preferred tag proteins include GST. A full discussion on the use of tag proteins can be found at Terpe et al., “Overview of tag protein fusions: from molecular and biochemical fundamentals to commercial systems,” Appl. Microbiol. Biotechnol. (2003) 60:523-533.

After purification, the tag proteins may optionally be removed from the expressed fusion protein, i.e., by specifically tailored enzymatic treatments known in the art. Commonly used proteases include enterokinase, tobacco etch virus (TEV), thrombin, and factor Xₐ.

### Immunogenic Compositions and Medicaments

The compositions described herein are preferably immunogenic compositions, and are more preferably vaccine compositions. In some embodiments, the pH of the composition is between 6 and 8, preferably about 7. The pH may be maintained by the use of a buffer. The composition may be sterile and/or pyrogen-free. The composition may be isotonic with respect to humans.

Vaccines as described herein may either be prophylactic (i.e. to prevent infection) or therapeutic (i.e. to treat infection), but will typically be prophylactic. Accordingly, methods are described for the therapeutic or prophylactic treatment of a S. pneumoniae infection in an animal susceptible to such S. pneumoniae infection comprising administering to said animal a therapeutic or prophylactic amount of the immunogenic composition described herein. For example, methods are described for the therapeutic or prophylactic treatment of a S. pneumoniae infection in an animal susceptible to streptococcal infection comprising administering to said animal a therapeutic or prophylactic amount of the immunogenic compositions described herein.

The compositions described herein can also be used as a medicament. In some embodiments, the medicament is preferably able to raise an immune response in a mammal (i.e. it is an immunogenic composition) and is, in some embodiments, a vaccine. The compositions described herein can also be used in the manufacture of a medicament for raising an immune response in a mammal. In some embodiments, the medicament is a vaccine.

The compositions described herein can also be use in kits containing one or more containers of the compositions. Such compositions can be in liquid form or can be lyophilized, as can individual antigens. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). Such compositions may comprise a first component comprising one or more S. pneumoniae Pili Polypeptides. Preferably, the S. pneumoniae Pili Polypeptides are in an oligomeric or hyperoligomeric form.

The kit can further contain a second container comprising a pharmaceutically-acceptable buffer, such as phosphate-buffered saline, Ringer's solution, or dextrose solution. It can also contain other materials useful to the end-user, including other buffers, diluents, filters, needles, and syringes. The kit can also contain a second or third container with another active agent, for example an antibiotic.

The kit can also contain a package insert containing written instructions for methods of inducing immunity against S. pneumoniae or for treating S. pneumoniae infections. The package insert can be an unapproved draft package insert or can be a package insert approved by the Food and Drug Administration (FDA) or other regulatory body.

A delivery device pre-filled with the immunogenic compositions described herein can also be used.

A method for inducing an immune response in a mammal can include the step of administering an effective amount of a composition as described herein. In some embodiments, the immune response is protective and preferably involves antibodies and/or cell-mediated immunity. This immune response will, in some embodiments, induce long lasting (e.g., neutralizing) antibodies and a cell mediated immunity that can quickly respond upon exposure to one or more S. pneumoniae antigens. In some embodiments, the method raise a booster response.

Methods of neutralizing S. pneumoniae infection in a mammal can include administering to the mammal an effective amount of the immunogenic compositions as described herein, a vaccine as described herein, or antibodies which recognize an immunogenic composition as described herein.

In some embodiments, the mammal is a human. Where the vaccine is for prophylactic use, the human can be a male or a female (either of child bearing age or a teenager). Alternatively, the human may be elderly (e.g., over the age of 50, 55, 60, 65, 70, 75, 80, or 85) and may have an underlying disease such as diabetes or cancer. In some embodiments, where the vaccine is for therapeutic use, the human can be a pregnant female or an elderly adult.

In some embodiments, the uses and methods described herein are for the prevention and/or treatment of a disease caused by S. pneumoniae. The compositions may also be effective against other streptococcal bacteria. The compositions may also be effective against other Gram positive bacteria.

Some methods of checking efficacy of therapeutic treatment involves monitoring S. pneumoniae bacterial infection after administration of a composition as described herein. A non-limiting means of checking efficacy of prophylactic treatment involves monitoring immune responses against the S. pneumoniae antigens in the compositions described herein after administration of the composition.

A non-limiting means of assessing the immunogenicity of the component proteins of the immunogenic compositions described herien is to express the proteins recombinantly and to screen patient sera or mucosal secretions by immunoblot. A positive reaction between the protein and the patient serum indicates that the patient has previously mounted an immune response to the protein in question—that is, the protein is an immunogen. This method may also be used to identify immunodominant proteins and/or epitopes.

Another means of checking efficacy of therapeutic treatment involves monitoring S. pneumoniae infection after administration of the compositions described herein. One way of checking efficacy of prophylactic treatment involves monitoring immune responses both systemically (such as monitoring the level of IgG1 and IgG2a production) and mucosally (such as monitoring the level of IgA production) against the S. pneumoniae antigens in the compositions described herein after administration of the composition. Typically, S. pneumoniae serum specific antibody responses are determined post-immunization, but pre-challenge, whereas mucosal S. pneumoniae specific antibody body responses are determined post-immunization and post-challenge.

The vaccine compositions described herein can, in some embodiments, be evaluated in in vitro and in vivo animal models prior to host, e.g., human, administration.

The efficacy of immunogenic compositions described herein can also be determined in vivo by challenging animal models of S. pneumoniae infection, e.g., guinea pigs or mice, with the immunogenic compositions. The immunogenic compositions may or may not be derived from the same serotypes as the challenge serotypes. In some embodiments, the immunogenic compositions are derivable from the same serotypes as the challenge serotypes. In some embodiments, the immunogenic composition and/or the challenge serotypes are derivable from the group of S. pneumoniae serotypes.

In vivo efficacy models include but are not limited to: (i) a murine infection model using human S. pneumoniae serotypes; (ii) a murine disease model which is a murine model using a mouse-adapted S. pneumoniae strain, such as those strains which are particularly virulent in mice and (iii) a primate model using human S. pneumoniae isolates.

The immune response may be one or both of a TH1 immune response and a TH2 response. The immune response may be an improved or an enhanced or an altered immune response. The immune response may be one or both of a systemic and a mucosal immune response. In some embodiments the immune response is an enhanced system and/or mucosal response. An enhanced systemic and/or mucosal immunity is reflected in an enhanced TH1 and/or TH2 immune response. In some embodiments, the enhanced immune response includes an increase in the production of IgG1 and/or IgG2a and/or IgA. In some embodiments, the mucosal immune response is a TH2 immune response. In some embodiments, the mucosal immune response includes an increase in the production of IgA.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. In some embodiments, the enhanced TH2 immune response will include an increase in IgG1 production.

A TH1 immune response may include one or more of an increase in CTLs, an increase in one or more of the cytokines associated with a TH1 immune response (such as IL-2, IFNγ, and TNFβ), an increase in activated macrophages, an increase in NK activity, or an increase in the production of IgG2a. In some embodiments, the enhanced TH1 immune response will include an increase in IgG2a production.

The immunogenic compositions described herein, in particular, immunogenic compositions comprising one or more S. pneumoniae Polypeptide antigen, may be used either alone or in combination with other antigens optionally with an immunoregulatory agent capable of eliciting a Th1 and/or Th2 response.

The compositions described herein will generally be administered directly to a patient. Certain routes may be favored for certain compositions, as resulting in the generation of a more effective immune response, in some embodiments, a CMI response, or as being less likely to induce side effects, or as being easier for administration. Direct delivery may be accomplished by parenteral injection (e.g. subcutaneously, intraperitoneally, intradermally, intravenously, intramuscularly, or to the interstitial space of a tissue), or by rectal, oral (e.g. tablet, spray), vaginal, topical, transdermal (see, e.g., WO 99/27961) or transcutaneous (see, e.g., WO 02/074244 and WO 02/064162), intranasal (see, e.g., WO03/028760), ocular, aural, pulmonary or other mucosal administration.

The compositions described herein may be used to elicit systemic and/or mucosal immunity.

In some embodiments, the immunogenic composition comprises one or more S. pneumoniae Polypeptide antigen(s) which elicits a neutralizing antibody response and one or more S. pneumoniae Polypeptide antigen(s) which elicits a cell mediated immune response. In this way, the neutralizing antibody response prevents or inhibits an initial S. pneumoniae infection while the cell-mediated immune response capable of eliciting an enhanced Th1 cellular response prevents further spreading of the S. pneumoniae infection. The immunogenic composition may include one or more S. pneumoniae Polypeptide antigens; one or more S. pneumoniae pilus or other S. pneumoniae antigens; and one or more non-pilus S. pneumoniae antigens, e.g., cytoplasmic antigens. In some embodiments, the immunogenic composition comprises one or more S. pneumoniae surface antigens or the like and one or other antigens, such as a cytoplasmic antigen capable of eliciting a Th1 cellular response.

Dosage treatment can be a single dose schedule or a multiple dose schedule. Multiple doses may be used in a primary immunization schedule and/or in a booster immunization schedule. In a multiple dose schedule the various doses may be given by the same or different routes e.g. a parenteral prime and mucosal boost, a mucosal prime and parenteral boost, etc.

The compositions described herein may be prepared in various forms. For example, the compositions may be prepared as injectables, either as liquid solutions or suspensions. Solid forms suitable for solution in, or suspension in, liquid vehicles prior to injection can also be prepared (e.g. a lyophilized composition). The composition may be prepared for topical administration e.g. as an ointment, cream or powder. The composition may be prepared for oral administration e.g. as a tablet or capsule, as a spray, or as a syrup (optionally flavored). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilized antigens.

Immunogenic compositions used as vaccines comprise an immunologically effective amount of antigen(s), as well as any other components, such as antibiotics, as needed. By ‘immunologically effective amount’, it is meant that the administration of that amount to an individual, either in a single dose or as part of a series, is effective for treatment or prevention, or increases a measurable immune response or prevents or reduces a clinical symptom. This amount varies depending upon the health and physical condition of the individual to be treated, age, the taxonomic group of individual to be treated (e.g. non-human primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

### Further Components of the Composition

The compositions described herein can, in addition to the components mentioned above, comprise one or more ‘pharmaceutically acceptable carriers’, which include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. A thorough discussion of pharmaceutically acceptable excipients is available in Gennaro (2000) Remington: The Science and Practice of Pharmacy. 20th ed., ISBN: 0683306472.

### Adjuvants

Vaccines as described herein may be administered in conjunction with other immunoregulatory agents. In some embodiments, compositions further comprise one or more adjuvants. Adjuvants for use with the vaccines described herein include, but are not limited to, one or more of the following set forth below:

### Mineral Containing Compositions

Mineral containing compositions suitable for use as adjuvants with the compositions disclosed herein include mineral salts, such as aluminum salts and calcium salts. Including mineral salts such as hydroxides (e.g. oxyhydroxides), phosphates (e.g. hydroxyphosphates, orthophosphates), sulfates, etc. (see, e.g., chapters 8 & 9 of Vaccine Design . . . (1995) eds. Powell & Newman. ISBN: 030644867X. Plenum.), or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. The mineral containing compositions may also be formulated as a particle of metal salt (WO 00/23105).

Aluminum salts may be included in vaccines described herein such that the dose of Al³⁺ is between 0.2 and 1.0 mg per dose.

### Oil-Emulsions

Oil-emulsion compositions suitable for use as adjuvants with the compositions described herein include squalene-water emulsions, such as MF59 (5% Squalene, 0.5% Tween 80, and 0.5% Span 85, formulated into submicron particles using a microfluidizer). See WO90/14837. See also, Podda, “The adjuvanted influenza vaccines with novel adjuvants: experience with the MF59-adjuvanted vaccine”, Vaccine (2001) 19: 2673-2680; Frey et al., “Comparison of the safety, tolerability, and immunogenicity of a MF59-adjuvanted influenza vaccine and a non-adjuvanted influenza vaccine in non-elderly adults”, Vaccine (2003) 21:4234-4237. MF59 is used as the adjuvant in the FLUAD™ influenza virus trivalent subunit vaccine.

In some embodiments adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80™ (polyoxyelthylenesorbitan monooleate), and/or 0.25-1.0% Span 85™ (sorbitan trioleate), and, optionally, N-acetylmuramyl-L-alanyl-D-isogluatminyl-L-alanine-2-(1′-2′-dipalmitoyl-sn-glycero-3-huydroxyphosphosphoryloxy)-ethylamine (MTP-PE), for example, the submicron oil-in-water emulsion known as “MF59” (International Publication No. WO 90/14837; U.S. Pat. Nos. 6,299,884 and 6,451,325, incorporated herein by reference in their entireties; and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296). MF59 contains 4-5% w/v Squalene (e.g. 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in International Publication No. WO 90/14837 and U.S. Pat. Nos. 6,299,884 and 6,451,325, incorporated herein by reference in their entireties.

Complete Freund's adjuvant (CFA) and incomplete Freund's adjuvant (IFA) may also be used as adjuvants with the compositions disclosed herein.

### Saponin Formulations

Saponin formulations, may also be used as adjuvants with the compositions disclosed herein. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using High Performance Thin Layer Chromatography (HP-TLC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO96/33739).

Combinations of saponins and cholesterols can be used to form unique particles called Immunostimulating Complexs (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP0109942, WO 96/11711 and WO 96/33739. Optionally, the ISCOMS may be devoid of additional detergent. See WO 00/07621.

A review of the development of saponin based adjuvants can be found at Barr, et al., “ISCOMs and other saponin based adjuvants”, Advanced Drug Delivery Reviews (1998) 32:247-271. See also Sjolander, et al., “Uptake and adjuvant activity of orally delivered saponin and ISCOM vaccines”, Advanced Drug Delivery Reviews (1998) 32:321-338.

### Virosomes and Virus Like Particles (VLPs)

Virosomes and Virus Like Particles (VLPs) can also be used as adjuvants with the compositions disclosed herein. These structures generally contain one or more proteins from a virus optionally combined or formulated with a phospholipid. They are generally non-pathogenic and non-replicating, and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Q13-phage (such as coat proteins), GA-phage, fr-phage, AP205 phage, and Ty (such as retrotransposon Ty protein p1). VLPs are discussed further in WO 03/024480, WO 03/024481, and Niikura et al., “Chimeric Recombinant Hepatitis E Virus-Like Particles as an Oral Vaccine Vehicle Presenting Foreign Epitopes”, Virology (2002) 293:273-280; Lenz et al., “Papillomarivurs-Like Particles Induce Acute Activation of Dendritic Cells”, Journal of Immunology (2001) 5246-5355; Pinto, et al., “Cellular Immune Responses to Human Papillomavirus (HPV)-16 L1 Healthy Volunteers Immunized with Recombinant HPV-16 L1 Virus-Like Particles”, Journal of Infectious Diseases (2003) 188:327-338; and Gerber et al., “Human Papillomavirus Virus-Like Particles Are Efficient Oral Immunogens when Coadministered with Escherichia coli Heat-Labile Enterotoxin Mutant R192G or CpG”, Journal of Virology (2001) 75(10):4752-4760. Virosomes are discussed further in, for example, Gluck et al., “New Technology Platforms in the Development of Vaccines for the Future”, Vaccine (2002) 20:B10-B16. Immunopotentiating reconstituted influenza virosomes (IRIV) are used as the subunit antigen delivery system in the intranasal trivalent INFLEIXAL™ product {Mischler & Metcalfe (2002) Vaccine 20 Suppl 5:B17-23} and the INFLUVAC PLUS™ product.

### Bacterial or Microbial Derivatives

Adjuvants suitable for use with the compositions described herein include bacterial or microbial derivatives such as:

### (a) Non-Toxic Derivatives of Enterobacterial Lipopolysaccharide (LPS)

Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC-529. See Johnson et al. (1999) Bioorg Med Chem Lett 9:2273-2278.

### (b) Lipid A Derivatives

Lipid A derivatives include derivatives of lipid A from Escherichia coli such as OM-174. OM-174 is described for example in Meraldi et al., “OM-174, a New Adjuvant with a Potential for Human Use, Induces a Protective Response with Administered with the Synthetic C-Terminal Fragment 242-310 from the circumsporozoite protein of Plasmodium berghei”, Vaccine (2003) 21:2485-2491; and Pajak, et al., “The Adjuvant OM-174 induces both the migration and maturation of murine dendritic cells in vivo”, Vaccine (2003) 21:836-842.

### (c) Immunostimulatory Oligonucleotides

Immunostimulatory oligonucleotides suitable for use as adjuvants with the compositions described herein include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine may be replaced with an analog such as 2′-deoxy-7-deazaguanosine. See Kandimalla, et al., “Divergent synthetic nucleotide motif recognition pattern: design and development of potent immunomodulatory oligodeoxyribonucleotide agents with distinct cytokine induction profiles”, Nucleic Acids Research (2003) 31(9): 2393-2400; WO02/26757 and WO99/62923 for examples of possible analog substitutions. The adjuvant effect of CpG oligonucleotides is further discussed in Krieg, “CpG motifs: the active ingredient in bacterial extracts?”, Nature Medicine (2003) 9(7): 831-835; McCluskie, et al., “Parenteral and mucosal prime-boost immunization strategies in mice with hepatitis B surface antigen and CpG DNA”, FEMS Immunology and Medical Microbiology (2002) 32:179-185; WO98/40100; U.S. Pat. No. 6,207,646; U.S. Pat. No. 6,239,116 and U.S. Pat. No. 6,429,199.

The CpG sequence may be directed to TLR9, such as the motif GTCGTT (SEQ ID NO:22) or TTCGTT (SEQ ID NO:23). See Kandimalla, et al., “Toll-like receptor 9: modulation of recognition and cytokine induction by novel synthetic CpG DNAs,” Biochemical Society Transactions (2003) 31 (part 3): 654-658. The CpG sequence may be specific for inducing a Th1 immune response, such as a CpG-A ODN, or it may be more specific for inducing a B cell response, such a CpG-B ODN. CpG-A and CpG-B ODNs are discussed in Blackwell, et al., “CpG-A-Induced Monocyte IFN-gamma-Inducible Protein-10 Production is Regulated by Plasmacytoid Dendritic Cell Derived IFN-alpha”, J. Immunol. (2003) 170(8):4061-4068; Krieg, “From A to Z on CpG”, TRENDS in Immunology (2002) 23(2): 64-65 and WO01/95935. Preferably, the CpG is a CpG-A ODN.

In some embodiments, the CpG oligonucleotide is constructed so that the 5′ end is accessible for receptor recognition. Optionally, two CpG oligonucleotide sequences may be attached at their 3′ ends to form “immunomers”. See, e.g., Kandimalla, et al., “Secondary structures in CpG oligonucleotides affect immunostimulatory activity”, BBRC (2003) 306:948-953; Kandimalla, et al., “Toll-like receptor 9: modulation of recognition and cytokine induction by novel synthetic GpG DNAs”, Biochemical Society Transactions (2003) 31(part 3):664-658; Bhagat et al., “CpG penta- and hexadeoxyribonucleotides as potent immunomodulatory agents” BBRC (2003) 300:853-861 and WO 03/035836.

### (d) ADP-Ribosylating Toxins and Detoxified Derivatives Thereof.

Bacterial ADP-ribosylating toxins and detoxified derivatives thereof may be used as adjuvants with the compositions described herein. In some embodiments, the protein is derived from E. coli (i.e., E. coli heat labile enterotoxin “LT), cholera (“CT”), or pertussis (“PT”). The use of detoxified ADP-ribosylating toxins as mucosal adjuvants is described in WO95/17211 and as parenteral adjuvants in WO98/42375. In some embodiments, the adjuvant is a detoxified LT mutant such as LT-K63, LT-R72, and LTR192G. The use of ADP-ribosylating toxins and detoxified derivatives thereof, particularly LT-K₆₃ and LT-R72, as adjuvants can be found in the following references, each of which is specifically incorporated by reference herein in their entirety: Beignon, et al., “The LTR72 Mutant of Heat-Labile Enterotoxin of Escherichia coli Enhances the Ability of Peptide Antigens to Elicit CD4+ T Cells and Secrete Gamma Interferon after Coapplication onto Bare Skin,” Infection and Immunity (2002) 70(6):3012-3019; Pizza, et al., “Mucosal vaccines: non toxic derivatives of LT and CT as mucosal adjuvants,” Vaccine (2001) 19:2534-2541; Pizza, et al., “LTK63 and LTR72, two mucosal adjuvants ready for clinical trials,” Int. J. Med. Microbiol. (2000) 290(4-5):455-461; Scharton-Kersten et al., “Transcutaneous Immunization with Bacterial ADP-Ribosylating Exotoxins, Subunits and Unrelated Adjuvants,” Infection and Immunity (2000) 68(9):5306-5313; Ryan et al., “Mutants of Escherichia coli Heat-Labile Toxin Act as Effective Mucosal Adjuvants for Nasal Delivery of an Acellular Pertussis Vaccine: Differential Effects of the Nontoxic AB Complex and Enzyme Activity on Th1 and Th2 Cells,” Infection and Immunity (1999) 67(12):6270-6280; Partidos et al., “Heat-labile enterotoxin of Escherichia coli and its site-directed mutant LTK63 enhance the proliferative and cytotoxic T-cell responses to intranasally co-immunized synthetic peptides,” Immunol. Lett. (1999) 67(3):209-216; Peppoloni et al., “Mutants of the Escherichia coli heat-labile enterotoxin as safe and strong adjuvants for intranasal delivery of vaccines,” Vaccines (2003) 2(2):285-293; and Pine et al., (2002) “Intranasal immunization with influenza vaccine and a detoxified mutant of heat labile enterotoxin from Escherichia coli (LTK63),” J. Control Release (2002) 85(1-3):263-270. Numerical reference for amino acid substitutions is preferably based on the aligmnents of the A and B subunits of ADP-ribosylating toxins set forth in Domenighini et al., Mol. Microbiol. (1995) 15(6):1165-1167, specifically incorporated herein by reference in its entirety.

### Bioadhesives and Mucoadhesives

Bioadhesives and mucoadhesives may also be used as adjuvants with the compositions described herein. Suitable bioadhesives include esterified hyaluronic acid microspheres (Singh et al. (2001) J. Cont. Rele. 70:267-276) or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants with the compositions described herein. See, e.g., WO 99/27960.

### Microparticles

Microparticles may also be used as adjuvants with the compositions described herein. Microparticles (i.e. a particle of ˜100 nm to ˜150 μm in diameter, more preferably ˜200 nm to 30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide co glycolide) are preferred, optionally treated to have a negatively-charged surface (e.g. with SDS) or a positively-charged surface (e.g. with a cationic detergent, such as CTAB).

### Liposomes

Examples of liposome formulations suitable for use as adjuvants are described in U.S. Pat. No. 6,090,406, U.S. Pat. No. 5,916,588, and EP 0 626 169.

### Polyoxyethylene Ether and Polyoxyethylene Ester Formulations

Adjuvants suitable for use with the compositions described herein include polyoxyethylene ethers and polyoxyethylene esters. See WO99/52549. Such formulations further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO 01/21152).

In some embodiments polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### Polyphosphazene (PCPP)

PCPP formulations are described, for example, in Andrianov et al., “Preparation of hydrogel microspheres by coacervation of aqueous polyphophazene solutions”, Biomaterials (1998) 19(1-3):109-115 and Payne et al., “Protein Release from Polyphosphazene Matrices”, Adv. Drug. Delivery Review (1998) 31(3):185-196.

### Muramyl Peptides

Examples of muramyl peptides suitable for use as adjuvants with the compositions described herein include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-sn-glycero-3-hydroxyphosphoryloxy)-ethylamine MTP-PE).

### Imidazoquinolone Compounds.

Examples of imidazoquinolone compounds suitable for use adjuvants with the compositions described herein include Imiquamod and its homologues, described further in Stanley, “Imiquimod and the imidazoquinolones: mechanism of action and therapeutic potential,” Clin Exp Dermatol (2002) 27(7):571-577 and Jones, “Resiquimod 3M,” Curr Opin Investig Drugs (2003) 4(2):214-218.

The compositions described herein may also include combinations of the adjuvants identified above. For example, the following adjuvant compositions may be used with the compositions described herein:

(1) a saponin and an oil-in-water emulsion (WO 99/11241);

(2) a saponin (e.g., QS21)+a non-toxic LPS derivative (e.g. 3dMPL) (see WO 94/00153);

(3) a saponin (e.g., QS21)+a non-toxic LPS derivative (e.g. 3dMPL)+a cholesterol;

(4) a saponin (e.g. QS21)+3dMPL+IL-12 (optionally+a sterol) (WO 98/57659);

(5) combinations of 3dMPL with, for example, QS21 and/or oil-in-water emulsions (See European patent applications 0835318, 0735898 and 0761231);

(6) SAF, containing 10% Squalane, 0.4% Tween 80, 5% pluronic-block polymer L121, and thr-MDP, either microfluidized into a submicron emulsion or vortexed to generate a larger particle size emulsion;

(7) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™);

(8) one or more mineral salts (such as an aluminum salt)+a non-toxic derivative of LPS (such as 3dPML).

(9) one or more mineral salts (such as an aluminum salt)+an immunostimulatory oligonucleotide (such as a nucleotide sequence including a CpG motif). Combination No. (9) is a preferred adjuvant combination.

### Human Immunomodulators

Human immunomodulators suitable for use as adjuvants with the compositions described herein include cytokines, such as interleukins (e.g. IL-1, IL-2, IL-4, IL-5, IL-6, IL-7, IL-12, etc.), interferons (e.g. interferon-γ), macrophage colony stimulating factor, and tumor necrosis factor.

Aluminum salts and MF59 are preferred adjuvants for use with injectable influenza vaccines. Bacterial toxins and bioadhesives are preferred adjuvants for use with mucosally-delivered vaccines, such as nasal vaccines.

The immunogenic compositions described herein may be administered in combination with an antibiotic treatment regime. In some embodiments, the antibiotic is administered prior to administration of the antigen as described herein or the composition comprising the one or more of the antigens as described herein.

In some embodiments, the antibiotic is administered subsequent to the administration of the one or more antigens as described herein or the composition comprising the one or more antigens as described herein. Examples of antibiotics suitable for use in the treatment streptococcal infections include but are not limited to penicillin or a derivative thereof or clindamycin or the like.

Each of the patents, patent applications, accession numbers and publications described herein is hereby incorporated by reference in its entirety.

Various modifications of the invention, in addition to those described herein, will be apparent to those of skill in the art in view of the foregoing description. Such modifications are also intended to fall within the scope of the appended embodiments. The present invention is further demonstrated in the following examples that are for purposes of illustration and are not intended to limit the scope of the present invention.

### EXAMPLES

### Materials and Methods

S. pneumoniae Chromosomal DNA Extraction

The S. pneumoniae strains listed in Table 1 were grown in 200 mL liquid culture (THYE medium) until the OD600 reached between 0.25 to 0.5. Next, the samples were centrifuged at 6000 RPM for 15-20 minutes (some pellets were stored at this point at −20° C.). The pellets were then resuspended with 2.7 mL (for a 3 mL final volume) of 50 mM EDTA at pH 8.0 and transferred to a 15 mL Falcon™ tube (BD Biosciences; Bedford, Mass.). 0.55 mL of freshly prepared Lysozyme (12 mg/mL Sigma L-6876 in 50 mM EDTA pH 8.0 (Sigma-Aldrich Co.; St. Louis, Mo.)) and 50 μL of 5000 U/mL Mutanolysin (Sigma M-9901) in water were then sequentially added to the Falcon™ tube. These samples were incubated for one hour at 37° C.

After incubation, 3.6 mL of Nuclei Lysis Solution (Wizard® Genomic DNA Purification Kit (Promega Corp.; Madison, Wis.)) was added and the samples were mixed by inversion six-times. The samples were then incubated for 5 minutes at 80° C., then cooled to room temperature. Next 184 of RNase solution (Wizard® Genomic DNA Purification Kit) was added and the samples were mixed by inversion ten-times. The samples were then incubated for thirty minutes at 37° C.

Each sample was then divided into six 1.2 mL Eppendorf tubes. To each Eppendorf tube was added 0.2 mL of Protein Precipitation Solution (Wizard® Genomic DNA Purification Kit) and mixed by inversion ten-times. Immediately after the tenth inversion, the samples were centrifuged at room temperature in an Eppendorf centrifuge for three minutes at full speed. The supernatants from the Eppendorf tubes were then pooled into a 15 mL Falcon tube (about 8 mL was typically recovered). Next 0.6 vol. of isopropanol was added and the samples were mixed by inversion ten-times.

Precipitated DNA was recovered by inserting a Pasteur pipet that had been heat sealed and bent in a flame into the Falcon tube and hooking the pellet. The precipitated DNA pellet was then washed with Ethanol 70%. Finally, the precipitated DNA pellet was dissolved into 3 mL of TE (10 mM Tris HCl, 1 mM EDTA, pH 8.0).

### PCR Assays

The details of PCR are well known to those of skill in the art. (See Mark A. Valasek, M. A., & Repa, J. J. (2005) Advan. Physiol. Educ. 29, 151-159; Ausubel, F. M., Brent, R., Kingston, R. E., Moore, D. D., Seidman, J. G., Smith, J. A. & Struhl, K, Current Protocols in Molecular Biology, Hoboken, N.J.: Wiley, 2005.). The specific parameters used herein are as follows (50 μL final volume of amplification):

PCR Amplification of Genomic DNA

Reagents

5 μL Buffer (10× concentrated)
0.5 μL BSA (0.5 mg/ml)
1 μL dNTP (10 mM concentrated)
1.5 μL oligo @ 10 μmol/μL concentration (15 pmoles)
1.5 μL oligo @ 10 μmol/μL concentration (15 pmoles)
0.25 μL Taq polymerase (F. Hoffmann-La Roche Ltd; Basel, Switzerland)
50 ng genomic DNA
water to 50 μL

Cycle Parameters:

94° C. for 3 minutes (one cycle)
94° C. for 30 seconds, 52° C. for 30 seconds, 72° C. for 1 minute and 20 seconds (6 cycles)
94° C. for 30 seconds, 58° C. for 30 seconds, 72° C. for 1 minute and 20 seconds (30 cycles)
72° C. for 8 minutes (one cycle)

### PCR Amplification of Bacterial (Colony) DNA

Reagents

5 μL Buffer (10× concentrated)
0.5 μL BSA (0.5 mg/ml)
1 μL dNTP (10 mM concentrated)
1.5 μL oligo @ 10 μmol/μL concentration (15 pmoles)
1.5 μL oligo @ 10 μmol/μL concentration (15 pmoles)
0.25 μL Taq polymerase (F. Hoffmann-La Roche Ltd; Basel, Switzerland)
40.25 μL water
bacteria

Cycle Parameters

94° C. for 8 minutes (one cycle)
94° C. for 30 seconds, 52° C. for 30 seconds, 72° C. for 1 minute and 20 seconds (6 cycles)
94° C. for 30 seconds, 58° C. for 30 seconds, 72° C. for 1 minute and 20 seconds (30 cycles)
72° C. for 8 minutes (one cycle)

### DNA Probe Preparation and Hybridization

DNA samples were initially prepared (for steps 1 and 2) in parallel with separate containers for Cy3 and Cy5 labeling.

### (1) Pre-Anneal Primer to DNA

One gig genomic DNA, 2.6 μL random nonamers (GE Healthcare; Buckinghamshire, UK (formerly Amersham Pharmacia)), and 1.5 μL spikes (control DNAs) were combined and water was added to a final volume of 29.5 μL. The mixture was heated to 70° C. for five minutes, then cooled to room temperature for five minutes. Finally, the mixture was centrifuged briefly.

### (2) Reaction

4 mL NEB2 buffer, 2 mL Nucleotide Mix (2 mM dATP, dGTP, dTTP and 1 mM dCTP) (individually obtained from GE Healthcare; Buckinghamshire, UK), and 2 mL dCTP Cy3 (or Cy5) (GE Healthcare; Buckinghamshire, UK (formerly Amersham Pharmacia) were added to the mixture from part (1). The combination was mixed gently and 2.5 mL DNA Polymerase I, Large (Klenow) Fragment (New England Biolabs, Inc.; Ipswich, Mass.) was added. The combination was gently mixed further then incubated at 37° C. for 2.5 hours. Finally, the mixture was centrifuged briefly.

The two labeling reaction solutions were then combined and unincorporated nucleotides were removed by running the solution through a Qiaquick PCR clean-up spin column (Qiagen, Inc.; Valencia, Calif.). The samples were eluted from the column with two 304 portions of EB buffer. Volume was reduced to 7.5 μL in a speed vacuum.

### (3) Pre-Hybridization

Aluminum slides were incubated for two hours at 42° C. in a 5×SSC, 50% formamide, 0.2% SDS solution. Then the slides were dipped in water three-times in one dish, and two more times in a separate dish. These prepared slides were blown dry with nitrogen.

### (4) Hybridization

The DNA samples from step 2 were heat denatured for two minutes at 95° C. Then the tube was spun quickly. A hybridization mix containing 1 vol. of version II buffer and 2 vol. 100% formamide (deionized) was prepared and 22.5 μL of this mix was added to each sample. Then the samples were mixed, spun briefly and incubated for one hour at 42° C. Finally, the slides prepared at step 3 were loaded with sample and incubated overnight at 42° C.

### (5) Post-Hybridization Washes

The slides prepared at step 4 were submerged in a 1×SSC/0.2% SDS solution at 55° C. (after cover slips were removed). The slides were then removed and placed in a cradle in a second tray containing the same solution. The slides were incubated in the second tray at room temperature for five minutes on an orbiting shaker. Next, the cradle and slides were transferred to a third tray containing a 0.1×SSC/0.2% SDS solution at 55° C. The cradle was dipped five times in the solution in the third tray and then incubated at room temperature for ten minutes. The slides were then transferred to a fourth tray containing the same solution as the third tray where the slides were again dipped five times and incubated at room temperature for five minutes. Next the cradle and slides were transferred to a fifth tray containing a 0.1×SSC solution at 55° C., where the cradle and slides were dipped five times. The cradle and slides were then transferred to a sixth tray containing the same solution as the fifth tray where the slides were again dipped five times. Finally, the cradle and slides were transferred to a seventh tray containing water, where the cradle and slides were dipped twice quickly and then dried in the cradle with nitrogen gas.

Microarrav Data Analysis Protocols for Comparative Genomic Hybridization of Genomic DNA of S. pneumoniae Strains

### Slide Acquisition

Two color images of hybridized slides were acquired with the microarray scanner ScanArray (PerkinElmer Life and Analytical Sciences; Shelton, Conn.) at 10 μm of resolution. Acquisition parameters (photomultiplier gain and laser power) were adjusted to maintain a comparable fluorescence level between the two fluorophores channels (measuring the intensity level of specific control spots and the mean intensity of all the spots).

### Image Analysis

Images were analyzed with the software Genepix 5.x (Molecular Devices Corp.; Sunnyvale, Calif.). Automatic and manually curated flagging procedures for no good quality spots were applied. From the acquired images, the Genepix software extracted the intensity levels and computed default statistical parameters for each feature spotted on the microarray slide.

### Data Management and Quality Check

Data management was performed by a local installation of BASE (BioArray Software Environment, rel 1.2.10, Lund University). The data analysis consisted of two phases:

(a) Background correction and normalization—In this procedure, the local background intensity was subtracted to each spot intensity. The intensities were then normalized to the mean or median intensity of all the spots. The background correction and the normalization procedure (plug-in rel. 1.4) were applied in BASE with the following parameters:
Experimental Normalization: Experimental Median
Experimental Background Background local
Threshold to correct (bkg stdev): 1
New values (bkg stdev): 1
Symmetric correction (CH1=CH2): Yes

(b) Data merging—Intensity values of repeated spots of a single slide or of repeated slides were merged computing the mean intensities of the separated two color channels. Spots that were flagged for low quality level or for background correction were filtered out. The algorithm also evaluated the probability of having different intensity levels between the averages of the two colors intensities (applying a T-test, plug-in rel. 1.7). The resulting dataset was stored in the BASE application and could be exported for other analyses.

### Clustering Analysis

The ratio of the two color intensity levels was transformed in log 2 scale.

The computed ratios were organized in a table dataset containing a column for each slide or group of merged slides (experiments), and a row for each spot or group merged spots (genes). The table was uploaded in the software TMeV (rel, 3.1, TIGR) for the clustering analysis.

Three main clustering algorithms were applied:

(a) Hierarchical clustering—This algorithm was applied to discover groups of genes showing similar presence/absence patterns among different experiments. In particular the algorithm was applied with the following settings of parameters:
Tree selection: Gene Tree and/or Sample Tree
Distance Metric Selection: Euclidean distance, Pearson uncentered
Linkage method selection: Average linkage clustering

A similarity tree (based on the unweighted pair group method with arithmetic mean algorithm, UPGMA) and a similarity matrix (based on the distance metric selected) were built to represent graphically and numerically the distances between experiments and between genes.

(b) Pavlidis template matching—This algorithm was applied to discover genes showing presence/absence patterns similar to a template pattern. Pavlidis template matching was applied to discover genes showing patterns correlated or anti-correlated to a reference gene among different experiments. The algorithm was applied using the threshold parameters Absolute R (selects patterns that are either correlated or anti-correlated with the template), a threshold p-value of 0.05, Pearson default distance metrics, and hierarchical clustering of the elements in the resulting matched and unmatched groups.

(c) Significance analysis of microarrays—This algorithm was applied to discover significant genes showing presence/absence patterns that able to divide the experiments in separated groups (defined a priori). The algorithm was applied to discover genes that were specific of a given group of experiments. The algorithm was applied with the following parameters:

Statistical test: Two-class unpaired

Number of permutations: 100

S0: Tusher et al. method

Q-values: No

Imputation engine: K-nearest neighbour imputer

Hierarchical clustering of the elements in the resulting matched and unmatched groups

### Example 1

### Determination of the Presence of the Pilus II Island (INV104B) in Genomic DNA

The determination of the presence of the pilus II island (INV104B) in the S. Pneumoniae strains listed in Table 1 was performed on purified genomic DNA or directly on bacteria (colony PCR). The four oligos used for amplification are listed in Table 2.

TABLE 2
PCR Oligos

| Designation | Direction | Sequence |
|---|---|---|
| 1008for | 5′ → 3′ | GCTGGATCGAGTTTGAAACCAGAA |
| | | (SEQ ID NO: 24) |
| 1009 rev | 3′ → 5′ | TAAGGATCACCAAAGTCCAAGGCA |
| | | (SEQ ID NO: 25) |
| Int-rev | 3′ → 5′ | TTTCAGTGTATGTTTTAGTGCTTCA |
| | | (SEQ ID NO: 26) |
| Int-for | 5′ → 3′ | ATGGCTTCAGGGGCTATGTTCGGTG |
| | | (SEQ ID NO: 27) |

Three diagnostic PCRs were performed for each strain listed in Table 1 with the following PCR Oligo combinations: 1008for-1009rev; 1008for-intrev; and intfor-1009rev. Clones positive for the pilus II island (INV104B) were successfully amplified by 1008for-intrev and intfor-1009rev. Strains negative for the pilus II island (INV104B) were successfully amplified by 1008for-1009rev.

The determination of the presence of the pilus island, i.e., the rlrA islet (as opposed to the pilus II island (INV104B)), in the S. Pneumoniae strains listed in Table 1 was performed. Similarly to the pilus II island (INV104B), specific primers were designed and used to amplify defined regions of the rlrA islet. Examples of methods for determining the presence of the rlrA islet are set forth in U.S. application Ser. No. 11/707,433, entitled “Purification of Bacterial Antigen,” filed Feb. 16, 2007; and U.S. Provisional Application Ser. No. 60/774,450, entitled “Purification of Bacterial Antigen,” filed on Feb. 17, 2006, the contents of which are incorporated herein by reference in their entirety.

Table 1 indicates that a great number of S. pneumoniae strains contain one or both pilus and pilus II island (INV104B).

### Example 2

### Sequences from 23F, INV200, and OXC141

1. Sequence Download and Assembly

The preliminary sequences of four strains of S. pneumoniae were downloaded from the Sanger Web site (see worldwide web site “sanger.ac.uk/Projects/Microbes/”). The Sanger sequences are composed by a variable number of non-overlapping contigs. Details for the downloaded sequences are listed in Table 3:

TABLE 3
Downloaded Sequences

| Strain | Serotype | ST | # of Contigs | Total bp |
|---|---|---|---|---|
| 23F | 23F | 81 | 21 | 2225211 |
| INV104B | 1 | 227 | 68 | 1986609 |
| INV200 | 14 | 9 | 167 | 2022487 |
| OXC141 | 3 | 180 | 120 | 1962139 |

To identify the likely ordering of the contigs, the sequences were aligned against the TIGR4 complete sequence using MUMmer3.19, to form a single pseudo-molecule. To separate two subsequent contigs the following sequence was inserted:

(SEQ ID NO: 28)
NNNNNCATTCCATTCATTAATTAATTAATGAATGAATGNNNNN

SEQ ID NO:28 was designed to (i) generate a stop codon in all six reading frames so that no gene is predicted across junctions and (ii) provide a start site in all frames, pointing toward contigs to predict incomplete genes at their extremities. Contigs that did not align against the TIGR4 genome were put at the end of the pseudochromosome.

2. Gene Prediction

The genes were predicted using the glimmer3.02 suite. The Hidden Markov Model was trained using the predicted genes of TIGR4 as a training set.

3. Protein Set Comparison

All the proteins of each genome were compared against all the proteins of the other genomes using Fasta. A protein was considered to be conserved if its aminoacidic sequence could be aligned with a percent identity of at least 90% on at least 50% of its length.

The amino acid sequences of OXC141, INV200, and 23F that did not meet this criteria are set forth below. These sequences may provide polypeptides and/or proteins that can be used in compositions or methods of treatment for, diagnosis of, and immunization against S. pneumoniae infections.

4. Start Codons

The N-terminal residues in the amino acid sequences listed herein are given as the amino acid encoded by the first codon in the corresponding nucleotide sequence. In those sequences in which the first codon is not ATG, it will be understood that the first codon will be translated as methionine when the codon is a start codon, but will be translated as the indicated non-Met amino acid when the sequence is at the C-terminus of a fusion partner. The listed sequences specifically disclose and encompass each of the amino acid sequences listed having a N-terminus methionine residue (e.g. a formyl methionine residue) in place of any indicated non-Met residue.

5. Sequences Identified from OXC141
>orf00007
(SEQ ID NO: 29)
MEMSFIAQDFDKLNIITVLESRTQAIIRNPMNTRLSSDTESSFNKIVRN
>orf00009
(SEQ ID NO: 30)
MELAETSIVKKNHQIPCIINQKIAQKLIKKTSMTDIDHQLSISTSTVIRKINDFHFEHDF
SRLPEIMS
>orf00013
(SEQ ID NO: 31)
LFKIGRVYYRQLQEDLLTCCNKYPKLFFFIIISLNSTQSGGVF
>orf00015
(SEQ ID NO: 32)
MKYNKTKYPNIYYYETAKGKRYYVRRSFFFRGKKREKSKSGFTTLPQARAALVELEQQIQ
EQELGINTNLTLDQYWDIYSEKRLSTGRWNDTSYYLNDNLYNNHIKAKFGSILLKNLDRN
EYELFIAEKLQNHTRYTVQTLNSSFMALLNDAVKNGNLLSNRLKGVFIGQSDIPAANKKV
TLKEFKTWIAKAEEIMPKQFYALTYLTIFGLRRGEVFGLRPMDITQNDSGRAILHLRDSR
SNQTLEGKGGLKTKDSERYVCLDDIGTDLIYYLIAEASKIKRKLGIIKEQHKDYITINEK
GGLINPNQLNRNFNLVNEATGLHVTPHMMRHFFTTQSIIAGVPLEQLSQALGHTKVYMTD
RYNQVEDELAEATTDLFLSHIR
>orf00016
(SEQ ID NO: 33)
MSYSYVALDVETANDFRGSVCSIGLVKFKDGNIVDTFYTLINPEEEFDDFNIFIHGITPE
DVLDSPTFPEVRKSIVDFIGLDIVVAHFAQFDMGALKDVYQKYELDFDNIEYICSYRLAK
VALPGQLNYKLKRLAKNLNIELDHHNALSDARASGLILEYLLSTNSFSDLTAFLKEYRYN
KTGLLGQYGFKRKKGYQYKENLIYQPTEEEKAAMNPDHYFYGLYFCFTGKLERMTRKEAN
KAAALVGGIPEKGVTKHTNILVVGEQDWRVVGTDGLSSKMKKAQTLLEKGQDIEIMTEND
FIRLLEE
>orf00017
(SEQ ID NO: 34)
MSLLSLHQCKVFLFYHDFISHGFKIVVGHEFEVIKLCGVIQAF
>orf00019
(SEQ ID NO: 35)
MKNKKPNAERLQEIADYFNVSTDYLLGRTDNPAIAGDSKEYTWQGKTLNVEEMASNVMMF
GGRELTDEKKKIIQSIIEGYLKEAGD
>orf00020
(SEQ ID NO: 36)
MSKLTKEDVLQVSQEIINDAIPVIKDMLDEVFKEYPIDMEIRKAILNSVLVAHKLSTETT
VSLLTELVNAQEN
>orf00021
(SEQ ID NO: 37)
MSKELKIIKANIKTRLIELDMTQAELAKQVSVASSVISELLKYGEGSESVREKVADVLGI
ENPWENS
>orf00022
(SEQ ID NO: 38)
MDYQILIQPAISVILAIISGLWSYIASKANNKAEIEKQAKEHSHIVEKLEKEFHYQIDTL
KQQHTLELEKVKQAHELRLQELEKVSQIDTETDKAMKMNDLIYKTFTGEVDLDKALKLAD
KANNHKQKLNKKFIQKTSKKS
>orf00023
(SEQ ID NO: 39)
MNEIFNFHGQEVRTLTIDDEPWFVGKDVADILGYSKARNAITLHVDEEDALKQGIPTSGG
TQDMLIINESGLYSLILSSKLPQAKEFKRWVTSEVLPAIRKQGGFIREDLDEDAFIALFT
GQKKLREQQATMLEDIDYLKSEQPIHPSYAQSLLKKRKARVVACLGGIDSPAYADKVFAQ
SVFRQAEIDFKDHFNISRYDLLPKKHADAALAYWMTWEPSTNTKMKIMKLNSFDDV
>orf00025
(SEQ ID NO: 40)
MFEPPILDQLMGVGALLLGFAGACRHIKLQEQRKEEERREEQEFASMIIQGYNHAYERGR
EAERQEIRKNIRRPFKGFTYDNEPPQGLRPEPLALPEPKQSAIRLL
>orf00026
(SEQ ID NO: 41)
MEELIESLDNLIMIVKELEGRESTSRHFITIWENDYKNLLLVKEYLTDYEKLAKDYRYVT
LKNKLLKIEKMELEGRHIYEDMRMKYRANRRKWGARYV
>orf00027
(SEQ ID NO: 42)
VLGMSEIKWIKITTDIFDDEKICLIDALPDPDAILVIWFKILTLAGKHNSNGLLMMTDKV
HYTDEMLATIFRRPLNTVRMAIGVFEQFGMIEIIDGIISLPNWEKHQNVDGMEKIKEQTR
NRVAKYRKKQKNLALGNVTGNVTVTDGNALEEDKDKNKNRLDKDKNKKRITTTSSGSEEN
ILELFQSEFRRLLSGFEIEEINHLLNENDVDLVKEALKTAINSGKPNIKYIGGILRNWQM
NNVTTVEQVRQSEKKNKDKKEEQEAKDEWGY
>orf00029
(SEQ ID NO: 43)
MRRSASMVDNVFEEIALSYRRNTEQQEEFCEKHNIPLIKILRTESVVCRMCESERIHEEN
QERVNELANAENERERKYYLEKFSLYDEVLKNATLDNFETPTEKEAEKLAFAKRICREWS
EGARNNIVLQGEAGTGKSHLAFAMVKVLSEYTKEIAIFINVTDLLMKIKADFSQEEFLVN
KIASAKFLVLDDLGMEKDSEWSFTILYNILNKRSNTIITTNLISADIQKRYGRPFMSRLM
KGVDKDHLMVFNDLTNKRKQYF
>orf00030
(SEQ ID NO: 44)
MLELYFVYNGHCKFFLGRFDNVDDLIEQMEDHQWAFSAITHPRFQKHIGQRTTRFDYGSK
DCYYLATFSGGE
>orf00031
(SEQ ID NO: 45)
MVGVTYQEIHLFVEFLKEQYGQGRPDYIEALNDLDGLVEVSYREAIERFLEDEVR
>orf00032
(SEQ ID NO: 46)
MMEELKKKVNAVYNWTVEDGKPQPPQQDLPQAVKDRVDYFWEMAEDGMTFMGAMECIFAD
EKPTDYELGATKGWLPKSKEFDDWIGYSPSMAQVVIAVYLIYGGN
>orf00033
(SEQ ID NO: 47)
MEETKMNKQELIKKLEERRTIIGNFQGYAVSYFWIYWIVEK
>orf00035
(SEQ ID NO: 48)
MEFVSPIKDNDDIQAMKDYLREWNEMYYMLFITGLNTGLRVGDILTLKVKDVQGWHIKLR
ERKTGKQITRRMTKELKKEMRRYVEGKPFHHFLFKSRQGQNKAITRERAYQIIHEAAEEL
GIDNVGTHTMRKTFGYKYYNKTKDVGTLQKMFNHSSPAITLRYIGIEQAELDDALRNFVI
>orf00036
(SEQ ID NO: 49)
MYNKPVRPSLKSKKWEKFRDRIMRKHDYLCQESLRYGISVQAEMVHHIFPVSEYPELEFV
EWNCLPLTNKKHNTFHDRVNDRVINQGLYWQKKRKKEFLNFFKNEK
>orf00037
(SEQ ID NO: 50)
LAKPITAKSIKSKVVKQMKDLGTYRKEFEMIIDIFAGMLYQYQKLAQDYANLGYPVTDTY
VNKAGAENERKVPILTAMEILRKDILSYSNQLMMNPKSLGEVVEQEGDSVLTEVLKFKNE
IKKKRVSGNG
>orf00040
(SEQ ID NO: 51)
MGNLDKAKEYAQHVLTHREEHCEENILAAERFFRDLENPAFEMDEDMVDFVIHFIENVIV
HQQGDDMFAVSIRNKPLLLQPWQHFVVVNLFGFYYKGTNERRFKEALIMLARKNGKTSFT
AAIALAYQILDTDSGSKCYIVANSVKQAMEAFGFLRFNVERWNDKNIRIKDNNQEHSITA
NFGDEGSFFIQALANDESRLDSLNGNVIILDEAHTMRNSKKHGLMKKTMSAYRNSMLFVI
STAGDIPTGFLANRLKYCQKVLKQLVTDDSFFIFICKANQSADGDVVNYLDENILKMANP
SWGVTVSLKALKEEAEQAMNDPQTRNEFFNKTLNIFTNSMNAYFNPDEFIASDSCYDWSL
EELARLPIRWYGGADLSRLHDLTAAALYGVYHDGEKDVDICITHAFFPRINAQKKANDDG
IPLFGWQSDGWLTMSNTPTVLYDDIVKWFIKMREKGFKIAAVGMDRKFGREFLTKMKQAR
FKMIDQPQLFYLKSEGFRRIEFKVKNKEFYYLHSDAYEYCVSNVRAIEKVDDAVQYEKLD
GDGGTARIDLFDASVFACIQALANLGKGGDVMRFFD
>orf00043
(SEQ ID NO: 52)
MNEIVLSEHEINLLINKGRVKVILNGEVVTIRQRHMKNLMAETVKWEKQVIDVSQNIVRN
KHFDSLFQNTFR
>orf00045
(SEQ ID NO: 53)
MGIFEKFWKRNKPSKPINMLSHSDLGLSNLMDSYVPLARNPDVVTAVNKIADLVSNMTIH
LMENTDKGDIRIRDGLARKIDINPCKHMTRKSWIFKIVRDLLLYGDGNSVLHVEYEPVTD
YISNLRPFPMREVSFQTDKDSYVISFRGEEYSPDEVVHFVINPDPDILYIGTGFRVTLTD
VVQSLNMATKTKKSFMNGKNIPSLIVKVDSSSAELDSEQGRERIAEKYLSTSRVGAPWIV
PEALLDIQQVKPLSLTDIALNESVELDKRTVAGLLGVPAFILGVGEFNKTEYNNFVNTTV
MSIATTITQTLTRDLLLSSNRYFKLNPRSLFSYNITELSAVAQQMANSAAMRRNEWRDWL
GMAPDPEMEELIVLENFLPQEKLGDQNKLKGGEEENAKAK
>orf00047
(SEQ ID NO: 54)
MQKRNSYRATQFQTREEESGDLVLSGYFIKFDEETELWRGYHEVIKRAGVEKAVTDADIR
ALFNHDDSLVLGRTGNGTLTLGVDDVGLFGDIIINKDDPQAVGAYARVKRGDVIGCSFGF
IPVKIETEEREDGSYLDTVLELEIFEVSPCTFPAYPQTEIAARQKDFESQKRANREALDK
RKKEIKEKFKL
>orf00048
(SEQ ID NO: 55)
MNKALIFGARMRAKATKVVELEETIEELNKRSVVELEKLDRAKNDEEVLAVEKTVDGLQR
EIEEKEAEKVQLENEIDELDKQIKEQNRKAPTPGKMEERGGKTLGQREAFNHYLRTKEAR
ADGFKSAEGEAIIPVELMTPKEAKQDKTDLTSLVNIVNVKNASGKWSVVKLTDQAMNTVE
ELEENPELAKPTFTKVNYEIKTRRGHLPVSQEFIDDADYDVMGLVAKQTKNQERITKNKE
IAKVLKTATSKSAAGLDGLKDILNVELKTYYNATIVCTQSMFAALDKIKDKDGRYMLQTD
ITSPTGYKFAGRVIVVYPDDIIGESKGDLKAFIGDVGEFATLFDRAQTTVKWQDDKIYGQ
YLATANRFDVVKVDGDAGFYVTYTDAVL
>orf00050
(SEQ ID NO: 56)
LMNVEDKDMSKDDTLTTEVEKEETKVVDGKPEEGDEE
>orf00051
(SEQ ID NO: 57)
MDNVQLLELLKLKLGIATKLRDKPLEKIIEAVKTELEDNLGVLLDLDSSEDQMFVVDFAA
FRYEGGVDMPRHLQWRLHNLQIASKKKVKNVES
>orf00052
(SEQ ID NO: 58)
MWNHEIKLISKKITGKDKLLQPISEDVEVTLLCRKKNVTRSEFYQANQAGLKPSLVVEIR
NFEYENQEFAKFEGKQYRILKTYPIDSEILELTLTEVLK
>orf00054
(SEQ ID NO: 59)
MSNDLADLIAKELAAYSDEVTEEVDKIAEQVTDETVDELKETSPKRYGKYRRSWKKKKLA
NGSFVVFNAVASLTHILENGHLSRNGGRVAGIVHIKPAEEKAIQNFEKRIKEIGK
>orf00055
(SEQ ID NO: 60)
MKLSDFAAILEQANLPVTYRAFKIGNAPDLPYLVYYESSPVINSADNTVNHQIKSVTVEL
AFESKDEDLEERLEELWANHKLFFEVQEETFIETERLYVKSYTVYLY
>orf00057
(SEQ ID NO: 61)
MTQENKVTFGLENVHIAPIKTLAADGVITYGDVFRFPGAMELILDTKGETTPIKADNKNY
HFMNSNEGYEGKLKIPHIIDEFATKILGEIKDPQTGVMTEKADASLTEFAIMFQFEGDKN
KTRYVMYYCFASRPSLGSKTKNGTSTNERELSFKASPRPLDTVVKRSITSADDKDAYDNW
FKKVYEPTAVAA
>orf00058
(SEQ ID NO: 62)
MRKIVLVGDQEYELGTNGYTPIAYKQQFGKDYFQDLFSMLKNQSFMNELNKLETDKELTA
TNIDISMLSDFDMTFFNRLFWTFAKSANPQIKPYEQFFMEMEIFPIQEVGPVLMEMLNAS
MTTKKHQMNQNQLAKKSSQ
>orf00060
(SEQ ID NO: 63)
MAGNIKGIKIEIDGDTQPLQKALKAINKESVNTTNELKQIDKALKFDTGNVILLTQKQEV
LQKQIGITRDKLETLRQAQSKVDEEFKKGNIGSEQYRAFQREVEVTQNVLKGYEGKLASL
TQALEGNGDAAKNNQAQLKELQNEQKLLASESEKVVSSFKLQESQMGANASEADKLALAE
KKIGAQSEIVTRQIENLEKQLSLTKEQYGENSAEANKMEAELNQAKTAYANLNQELGKLG
STAKSNQTQLKELQNEQSQLASEMSKVTSSFKLQESALGSNASEAERNALAQKKIGAQSE
IVSKQISNLEQQLEITKKEFGENSTQANKMESELNQAKTAFNHLNDEMKGTKSAADSTQE
SLSEISRNLRAELLQQFSEKLSAISEKLVEVGKEALEAAAQMQASNAQFTTVFGDMETQA
REALNAIGQEMDIVPERLQGSFTQMASFAKTSGLDTAEALDLTSRATRAAADGAAFYDKS
IESVTESLQSFLKGNFANDAALGISATETTRNAAANKLYGKSFKDLSEAQKQLTLLQMVE
DGNKLSGALGQAARESDGLENVMGNLKQAGTNALSAIGQPLLEMMIPVFQTLATIVKGVA
ELFSSLPAPVKDFVVILGTVVTAVGVIAPIFLSLQALAEFLKISIGEMIIAALPIIGTAI
AIAAAVAAIVAIVKYLWETNEGFRDAVTTVWNAILEVINAVVSEISNFVMSIFGTVVTWW
TENQELIRTSAETVWNAIYTVISTILDILGPLLQAGWDNIQLIITTTWEIIKIVVETAIN
VVLGVIQAVMQIITGDWSGAWETIKGVFSTVWQAIQSIVQTIFSAIQSYISNILNGISGT
VSNIWNSIKDTVSNVLNAISSTVSSVWEGIKSTISSAINGARDAVSSAIEAIKGLFNFNI
SWPHIPLPHFYVSGSANPLDWLSQGVPSIGIEWYAKGGIMTKPTIFGMNGNNIMVGGEAG
NEAVLPLNDKTLGAIGRGIAQTMGGTSPTINITITGNTVREEADISRIADEVAQRIADEL
QRKTQLRGGFT
>orf00064
(SEQ ID NO: 64)
MIKHNELVIDGVRTSSFPFKVIVHDSPSIALGESKTALLEHGGISGAIVQTNKHRELVKK
TYTIYLVKPTEEQMNQFMSLFIREKFWLESERVKTTRLWCYKVNVSDLEEVQPGLYMTKA
TFTCHPTKHFKVTDTQRLTRSGTLNVQGSALAFPKITIVGQSASETSFTIAGQVIRLERL
TESLVMVNNPDNPSFKTTTGKPVKWSGDFITVDPAKVKNIGVVLGPGIQSLEIETVWGWA
>orf00068
(SEQ ID NO: 65)
LLYLLNEDVRTVRWNGESLHEATSAIVKETMNGDFTLTVKYPISDSGIYQLIQEDMLIKA
PTPVLGAQLFRIKKPVEHNDHLEITAYHISDDVMQRSITQMSVTSQSCGMALSRMVQNTK
TALGDFSFNSDIQDRRTFNTTETETLYSVLLDGKHSIVGTWEGELVRDNFAMTVKKSRGE
NRGVVITTHKNLKDYQRTKNSQNVVTRIHAKSTFKPEGAEKETTIRVTVDSPLINSYPYI
NEKEYENNNAKTVEELQKWAQSKFSNEGIDKVSDAIKIEAYELDGQVVHMGDTVNLKSWK
HNVDAFKKAIAYEFDALKEEYISLTFDDKAGIGGSRASGGLSSAADAILGVTESAQEIAL
EKALQNADLDFDHKAGLLRQEISDDIELAKARAEEVKRELSDTINQRFNSFDNGPLKETK
RKAEEALRNAGASSSLAQESKRIGLDSVARLEAFKSQTTSAQTALSGDLDALKRTIANDI
RPKQAQAEAEIAKQVEALSRTKNELAGASTLLAQEAKRIELDSVARLEAFKSQTTSAQTA
LSGDLDALKRTIANDIRPKQAQAEAEIAKQVEALSRTKNELAGASTLLAQEAKRIELDSV
ARLEAFKSQTTSAQTALSGDLDALKRTIVNDIRPKQAQAEAEIAKQVEALSRTKNELAGV
KSAQATYKETTTRRLSELTNLANGKASKSELTQTAEELSSKIASVQASGRNLFLNSLFKQ
DIPKTGIWTTSTYTATIDSESKYLGHKALKIIGLNPSGRDGGNPKVTYPALGQFGKVIPG
STTNQDVTISFYAKANKNGIMLRSRLGNIGYKTGNVTLSTEIKRYAVHIPKGWTNESKRT
TNEWLFNFNQEGTVWIWMPKFEISDVDTSYSEAPEDIEGQISTVESTFKQRANSLEAGVS
RLTEGLRTKADISSLNVTAENIRQSVKSLETDTQNKLNQKLSQAEFEVRAGSIRQEILNA
TKDKADKTLVVSEAGKLREEFSKMKVGGRNLWIKSKTVGAVIEKLPENHVTGQKECYRLE
NNSTLTFNLEPDFSSRLYQKVTFSAWIKYENVVQGRNFWNVFNCFKHYLFRKNSETGVQS
GPDYDTLGRYKGSADWKYITFTYDYSEKTNFDQLKTLLRFNLEGATSGTAWVTGIKVEIG
SVATDWSPAPEDADGLITEAKATFERTAQGLRTDLSAITEGLRTKVDISALNVTAENIRQ
SVKSLETDTQNKLNQKLSQAEFEVRAGSIRQEILNATKDKADKTLVVSEAGKLREEFSKM
KVGGRNLWIKSKTVGAVIEKLPENHVTGQKECYRLENNSTLTFNLEPDFSSRLYQKVTFS
AWIKYENVVQGRNFWNVFNCFKHYLFRKNSETGVQSGPDYDTLGRYEGSADWKYITFTYD
YSEKTNFDQLKTSLRFNLEGATSGTAWVTGIKVEIGSVATDWSPAPEDADGLITEAKATF
ERTAQGLRTDLSAIQEYVNKDGQRQEALQRYTREESTRQATAVRELVNRDFVGKATYQED
VKGINQRIEAVKTSANKDIASQIASYRQSVDGKFTDISSQITTYKQDVGGQISGLSNRLT
SSEQGTTTQISNLSNRINSNKQGTDNQISNLKTQVATNKDNAERQMGRISDQVSANKANA
DSQFANVTNQLARKVETTDFQRVKETSKLYERILGNTENGIADKVARMALTNQLFQVEVA
KNASNGQNLLKGTKDFSGGWKNKGANWKKHAEKYKGVDVLFKNNSWNGVGQEIDAKIGEV
YTFSLWMKSDWKNDTVNFYVNRNGSVEKGWGVPSETSVAITSEWKRYSFAFKITVDGFIF
PRVERLNQNTNLYIAGLKLEKGSYATPYTEAPEDTDEAIRSVQSQLTGSWAVQNINSAGD
IISGINLGANGHNRFVGKLTHITGETLIDRAVIKSAMVDKLKTANFEAGSVTTTILDAEA
VTADKVRFDAAFIRKMTANDAFIDQLTSKRIFSTKVESVISSSTFLEAYQGRIGGFTIGR
FAQGRGRWISGINQFSVGMGNGEGGSYNGENTAFWANWGHSWNSPGPNAWYVTTSGNMYC
RNGADFHGKVDFSNSSRANFYGNTTFSRSPVFSNGIELGSKDVLGDGWNPKGGRNAVVWW
NQVGSGSVKYWMEQKSDRRLKENITDTAVKALDKINRLRMVAFDFIENKKHEEIGLIAQE
AETIVPRIVSRDPENPDGYLHIDYTALVPYLIKAIQELNQKIEKMEKTIA
>orf00069
(SEQ ID NO: 66)
MNTEQLNQALQMTIREMSTTSTNSMITSNILSIQLNEQREENQRLQARVDELEALLDEQT
KPADKGE
>orf00071
(SEQ ID NO: 67)
MAETIQNTDNLLDLTKITEPFDLASALRYMKENGEFIRCKNVSDDFYMYRDVQKRPVIVN
GRRQFKDIETVWAFNQWGGTITTINVAVLLNHEFYIMKFDAEGNPDWTNPTVEPKE
>orf00072
(SEQ ID NO: 68)
MQIEFFNFLRSVVQTEDGLVLYALALIVSMEIIDFVTGH
>orf00073
(SEQ ID NO: 69)
MILIPASVLLPEKTGFVFLHSIYLGYIAFTFQSLIENYRKLKGNVTLFQPIVKVFQRLLE
KDDDTKKGE
>orf00074
(SEQ ID NO: 70)
MQQITEIITNGAISILVILAGIAVKAVKEYLVKKGGEKTIKIVEILAKNAVNAVEQVAAE
TGYKGDEKLAQARAKVRAELTKYNISMTDKDLDTFVESAVKQMNDAWKGR
>orf00075
(SEQ ID NO: 71)
MAFNQFNRCVTLSIPTAPNIPTSVVHRTYLHDTAVSDNM
>orf00079
(SEQ ID NO: 72)
MKNREEEWQGIIAKNAILLIIAPFYFLIIVKNGVLLKIKTVTEITAF
>orf00085
(SEQ ID NO: 73)
VEEVEVAEVKNARVSLTGEKTKPMKLAEVTSINVNRTKTEMEEFTRVLGGGVVPGSLVLI
GGDPGIGKSTLLLQVSTQLSQVGTVLYVSGEESAQQIKLRAERLGDIDSEFYLYAETNMQ
SVRAEVERIQPDFLIIDSIQTIMSPEISGVQGSVSQVREVTAELMQLAKTNNIAIFIVGH
VTKEGTLAGPRMLEHMVDTVLYFEGERHHTFRILRAVKNRFGSTNEIGIFEMQSGGLVEV
LNPSQVFLEERLDGATGSSIVVTMEGTRPILAEVQALVTPTMFGNAKRTTTGLDFNRASL
IMAVLEKRAGLLLQNQDAYLKSAGGVKLDEPAIDLAVAVAIASIYKDKPTNPQECFVGEL
GLIGEIRRVNRIEQRINEAAKLGFTKIYVPKNSLTGITLPKEIQVIGVTTIQEVLKKVFA
>orf00088
(SEQ ID NO: 74)
MGVSIFLALFYMIPALYFLFHIGKKWELPKKVLILSLLGAICSFTSLLLFGIYNHRRKSS
KV
>orf00094
(SEQ ID NO: 75)
VFVALVSITFSLINFFKILINLTAQVSPQVIDEKILMMDLNLNNYLSTVIQLRQDVYTGI
KILH
>orf00095
(SEQ ID NO: 76)
VNIASLQNGHIFCWQVQHIANKLTSDFWIAEYFLSNQIISWADARMSENPPIPPSIVS
>orf00103
(SEQ ID NO: 77)
MKIKEQTRKLAAGCSKHCFKVVNGTDEVSSKHCFEVVDGTDEVSSKHCFEVVDRTDEVSN
HIYGKATLTWFEEIFEEYKSLHNKTHITKVV
>orf00109
(SEQ ID NO: 78)
MNDDDSRCIHIERDGKTIEFGYLNISSTDRNTSHADGLVGIFNSNFSGVRVRGIAVFLNG
PDNLDTTLVGNFQTIWNFRIICIHSQNTCNKG
>orf00110
(SEQ ID NO: 79)
LEFNFCRSIIKNGRDNLPNTNSTSGMATRWANHNWSDDIKDRLKTK
>orf00114
(SEQ ID NO: 80)
MRYDFGKVYKEIRESKGLTQEEVCGNVISRTSLSKIESGKATPKYENMEFLLRQINMSF
>orf00116
(SEQ ID NO: 81)
MNYNLKYLLSGIFVLVFIGFLVWMRYFNQRKEEEHSDVSFEARLDSEVKTLYKQLGLGEE
PHYFLAYRYLHPWFDLAILPPTVEIFLTKIALVMVTPDELLIRNLGNGLTFTSEHHRDLR
QGLIRIPKSEMKEFEIRNWKKFFVFGDFLTIKTSQHSYYLQVRDDGLQKGSLSTKHFSDL
KSQDFLGLLTDKRTF
>orf00124
(SEQ ID NO: 82)
LSLLDLRGSLCLRIYLHEPLITTVSQDFTSLSDISHF
>orf00125
(SEQ ID NO: 83)
MDFKSFIIGLVVGIFGPYMDDLIRKKFLKSSEKKTEKSVKK
>orf00175
(SEQ ID NO: 84)
MGRFILFENLFKPGQLHLAVDMVTDFVSSIHNLKTVF
>orf00177
(SEQ ID NO: 85)
METSISMADFYGKYQNENLELIDVREAHEFQAGHAPGAKNLPLSTLEQGYKELKPDHEYY
VICQGGVRSASTCQFLSSQGLTVTNVEGGMNVWPGQVE
>orf00179
(SEQ ID NO: 86)
LGGKSCLLEDRLCDIAAQTTVAADDVGLFFVQFISFLLDTLSVFDTIVQN
>orf00183
(SEQ ID NO: 87)
MKINDQTRKLAAGCSKHCFEVVDRTDEVSSKHCFEVADRTDEVSNIYTARRR
>orf00184
(SEQ ID NO: 88)
MKLLSIAISSYNAAAYLHYCVESLVIGGEQVGILIINDGSQDQTQEIAECLASKYPNIVR
AIYQENKGHGGVVNRGLAEASGRYFKVVDSDDWWILVPT
>orf00185
(SEQ ID NO: 89)
LKILETLQELESKGQEMDVFVTNFVYEKEGQSRKKSMSYESVLPVRQIFGWDQVGNFSKG
QYIMMHSLIYRTDLLRASQF
>orf00186
(SEQ ID NO: 90)
MYYLPVDFYRYLIGREDQSVNEQVMIKCIDQQLKVNRLLVDQLDLSQVSHPKMREYLLNH
IEITTVISSTLLNRSETAEHLAKKRQLWTYIQQENFEVFQAIRKTMLSRLTKHSVLPDRK
LSNVVYQITKSVYGFN
>orf00194
(SEQ ID NO: 91)
MSLQIKLKKLAKELSKLLKDSNLETVDKDVLENSQEELQKAVLFLADEKGSEHTAAELID
NLKEVIAKLKANA
>orf00202
(SEQ ID NO: 92)
MKIKEQTRKLAAGCSKHCFEVVDKTDEVSYIYLRQGEADAV
>orf00213
(SEQ ID NO: 93)
MSKEKVILAYSGGLDTSVAITWLKKDYDVVSVCMDVGEGKDLDFIHDKALKVGAVESYVI
DVKDEFATDYVLVALQSHAYYEQKYPLVSALSRPLISKKLVEIAHQTGATTIAHGCTGKG
NDQVEYQIAVAKKANEAKK
>orf00215
(SEQ ID NO: 94)
VLDSLVFMGFSMKLIHDLDTHTTHSTAKMLYNVKAIKNDFSIRE
>orf00218
(SEQ ID NO: 95)
MGQLHFITKLLDIKDTNTQIIDVVNRDSHKEIIAKLDYEAPSCPECRSQMKKYYFQKPSK
IPYLETTGMPTRILLRKRRFKCYHCSKMMVAETSIVKKNHQIPRIINQKIAQKLIEKISM
TDIAHQLSISTSTVIRKLNDFHFECNFRNLPKIMSWDVETVRGVTVSIGRWR
>orf00220
(SEQ ID NO: 96)
MRYDFGKVYKEIRESKGLTQEEVCGGVLSRTSLSKIESGKTTPKYENMEFLLRQINMSFE
EFEYICHLYQPSQRTEIMQTYLNMTSIIGSNSLVHFFETCQDYLKTHHDLPIEEIRDMLE
VVIYIRQHGAGELSDHAEQVVKKLWRKIEKQDTWYESDLKILNTILFSFPIEYLHLITGK
ILQRLEVYKNYQHLYDLRIAILLNLSTLYLYNQDKNMCKQICYTLLEDAKNKKSYDRLAI
CYVRIGICTDNAKLIQKGFSLLELTEETSMLSHLKKEVETHYQPKKL
>orf00221
(SEQ ID NO: 97)
MNSKELSISMLKKYPCTMQHDQSDCAAAVVSTVLLSYKKELSIMKIREIIGTDMYGTTVS
GIVSGLNKLNFTVKAVRVALEDLTPKLTFPAILQVKNDLGQNHFVVLHSIKRNSKFYVAD
PASGIRKMSSDELGEIYQGITLFMVPNSDFERGKLKGKGLLDLFGRLIFNQKGLISTVIL
ASFVLSIIGILSSLFSKVIMDEVIPYALKNSLYMFLIVFGIVSFLQTLLSAFRQHVLLFL
SRKIDIPVLMGYYDHIIHLPYSFFGSRRVGDVLTRFQDAMTIKNVFTSVSISLVMDITLS
VISAVVLWTINQSLFLILVFMVIVNIILIYCFKKPYKKINHEQMEANGLLNSQLIESIRN
IDTIKSQHDEEQRLNKIEEKFVHTLEIGYKEGVLQNIQSTISSMTSTMGGLLFMGVGALF
IIDGKMTIGDLLVFQTLSQYFTEPIQNLVGLQLTFQEVQVAVSRLQELMEVDREDIALDY
SIRDFTLCDDIEFKDVTFAYGSRPPVIKDFNLRIKQGEKIAFVGESGAGKSTLVRLLLRF
INPSEGKIRIGENDLSDLDYGKLRKKISYIPQTIELFTGTIIDNLKIGNPSVTYEDMVRV
CRIVGIHDTIQRLQNRYGSFVEEGGQNFSGGEKQRLAIARALLSKADLYIFDEATSNLDS
FSEQIIQDLIFNKIMDKTTIVVAHRLSTILRCDKICFLENGTIVEYGTHEELMAKNGKYA
RMVGLQSVQVNQQIQSQAVLDTEEVTYG
>orf00222
(SEQ ID NO: 98)
MAKLEVKDNKKLVLKSVICKKLHDTKVEDVDQEINKFHQHLQLLKAQIFGPLIVKSCGTT
IHDDGLITTDFEFYIQAHNAQQYSNIYDVQDSISVPYCLYVRFEDSPEYLQYAYSKLDLY
VYENDIQTDGIVYTVYVNSSPEKMVVDIFRPIVSL
>orf00223
(SEQ ID NO: 99)
MKLYNKSELRYSRIFFDKRPPAFAFILIISTAIILSGALVGAAYIPKNYIVKANGNSVIT
GTEFLSAIGSGKVVTLHKSEGDMVNAGDVIISLSSGQEGLQASSLNKQLEKLRAKEAIFQ
KFEQSLNEKYNHLSNSGEEQEYYGKVEYYLSQLNSENYNNGTQYSKIQDEYTKLNKITAE
RNQLDADLQTLQNELIQLQQQGDSSSLSDTTSDDDKAKLETKISEITTKIEALKTNITSK
NSEIDSQQSNIKDMNRTYNDPTSQAYNIYAQLISELGTARSNNNKSITELEANLGVATGQ
DKAHSILASNEGTLHYLVPLKQGMSIQQGQTIAEVSGKEKGYYVEAFVLASDISRVSKGA
KVDVAITGVNSQKYGTLKGQVRQIDSGTISQETKEGNISLYKVMIELETLTLKHGSETVI
LQKDMPVEVRIVYDKETYLDWILEMLSFKQ
>orf00224
(SEQ ID NO: 100)
MELVLPNNYVVIDEEEMMYLDGGAIYIPRWAITGAITGAAYAALAAAGGGGLQLVLASYG
LRSALVAGIVKGLGVLGIHIGNAFANTVIRSIASAGIGAGADWIFTNIIDGWDGRRDNQL
RIG
>orf00226
(SEQ ID NO: 101)
MKDDQKYLLAGLYSLLVAIFYFPLIESKGIFVSILMAVLLLYLIYFIATVIHIVIIKFIR
KKSFKYLVLYPFTYDGSWRFQPINLLYFPEMVRDVIPINLVQEYCQGQPYGLLKKMLKRI
RLSREISLLLATIIVYFFTHRILPLSVFTFIFSYILLFAQSYLGGNTVWIGNRRLIIDDE
FEKILLSKSYIKEISSARYSEYLTCEYKNLTPIILLAIFENLLDSYLIQNQSKVDLDIFY
KVLPLLYKEKYTMGFNYFVSLNYLLYKVGFLGIIYDNEALRDLSKQYLNKNISELQDGSF
EDGIQDAVASKQIVVINEFIACLNSRCVPSQYDRFFYKDRPYIFSRKSPIKG
>orf00227
(SEQ ID NO: 102)
MKNKRYFFDTILIILLLISTIFCVSPVFIKLDILGTPSHAILTFVLAIPLFYILSQCLHT
LLLLVSSIFCKLRPIYFYFIFVIIIGARKYYRILFHQLMGFSPGVAVFYKESQTTKNLFK
FYYFLYFTTLISYYFFFTFVYDKPLLLPLIPFSIIIALVQKLYRIENQQLFLLKSKVLTI
LESKKDCEFNLQDYHEIWKLQSKSELPCVALSYISLIKPYLSESVREQIDLLEVKRFKKI
NHPISLYGMLDVIKLNLYLRHYNEKNKYESMLKKILEVRPDFVLIEQNIDDSLNSSQPLS
LSLAISEIQLLLEVYMGIKHVSIRR
>orf00228
(SEQ ID NO: 103)
MIRKPIIFLLMLPIWGLWIELHLLVSNLQLNLEIPFDFVVSTSLTFFVLILSKIVLDILY
ALKDLYKKEALITIFPFIFIRRKKVNVRFSPYFSFHRKSLSPDDLRSRIIWSFILEIAII
LVFILKIPFAIIMLTTIFFWTIMDINHLVFNKTEFLFNQNKWQKEDSFESDLTKTLKDKI
QKSELSYSDLMSLQLYDAMNQSTFLTDSELFEDILKKIEDSHNTLLCTGLVELLLYEMSI
SNNNNWQEKVDKIRIQLIRINQLDFFYYTSWLRQNFDFCMNREYHKMKSRKLLLSNKKIV
>orf00229
(SEQ ID NO: 104)
MELVLPNNYVVIDEEEMMYLDGGAYLSKRACQGICVALAMSPGTFIALTGAAVLTKKLIN
YIKVGGLGGWLIGAAAGVLAGAAGRIAYCIGYGALNRGCDISGNPYPWDGFISATVR
>orf00230
(SEQ ID NO: 105)
LGWIHICDSKMSNVDKIRKIHIIVCWVYIFLSFRAIINDTEYFLLIFLAFIYSIVSLPLY
SVKNKIVSICLVINSILLMSFPILINKFFPESFSTYIVLISVFITELIIFHLIGKDFDTK
LTNEYKKISQFRSKVSQSPWIKYLEISSFILTIFPSILYGTVDNHVLTLIFLIKICVDTT
IKFLFIRLFDTSTLMKRRIFFLFALDVIAYLFLGYLLVIQKAGYLFSVLLLFSNFSVPFI
KAKEYELFKNSK
>orf00232
(SEQ ID NO: 106)
MNKKKMILTSLASVAILGAGFVASSPTFVRAEEAPQVVEKSSLEKKYEEAKAKYDAAKKD
YDEAKKKAAEAQKKYEEDQKKTEEKAKKEKEAAKEVDDASLAVQKAHVEYRKVLDSRNSY
RNPSDYAKKLAEADKKITEETTKLTNAQTKFQSIRTTIVVPGQSELAETKKKAEEAKAEE
KVAKRKYDYATLKVALAKKEVEAKELEIEKLQDEISTLEQEVATAQHQVDNLKKLLAGVD
PDDTEAIEAKLKKGEAELNAKQAELAKKQTELEKLLDSLDPEGKTQDELDKEAAEAELNK
KVESLQNKVADLEKEISNLEILLGGADSEDDTAALQNKLATKKAELAKKQTELEKLLDSL
DPEGKTQDELDKEAAEAELDKKVESLQNKVADLEKEISNLEILLGGADSEDDTAALQNKL
ATKKAELEKTQKELDAALNELGPDGDEEETPAPAPQPEQPAPAPKPEQPAPAPKPEQPAP
APKPEQPAKPEKPAEEPTQPEKPATPKTGWKQENGMWYFYNTDGSMATGWLQNNGSWYYL
NANGSMATGWVKDGDTWYYLEASGAMKASQWFKVSDKWYYVNSNGAMATGWLQYNGSWYY
LNSNGAMATGWAKVNGSWYYLNANGSMATGWVKDGDTWYYLEASGAMKASQWFKVSDKWY
YVNSNGAMATGWLQYNGSWYYLNSNGAMATGWAKVNGSWYYLNANGSMATGWVKDGDTWY
YLEASGAMKASQWFKVSDKWYYVNSNGAMATGWLQYNGSWYYLNSNGAMATGWAKVNGSW
YYLNANGSMATGWVKDGDTWYYLEASGAMKASQWFKVSDKWYYVNGLGALAVNTTVDGYR
VNANGEWV
>orf00233
(SEQ ID NO: 107)
MKIRRRYTHIIRIICILTISFKKQFLSSSLSSLTKRVIMNTAQATFNREAHTTFNRE
>orf00252
(SEQ ID NO: 108)
LKKRMNRWQFLLNQSKEMVGILLLKVKEQELIEFVVNL
>orf00253
(SEQ ID NO: 109)
LIKVIKRKAFGFRNFNNFKKRILMTLNIKKESTNFVLSRL
>orf00257
(SEQ ID NO: 110)
MTYNEKRLINSLERGHMEQLKNTTDLLGLEDKNIKILSVLKYQTHLVVQAKLDSPAPPCP
HCQGKMIKYDFQKASKIPLLDCQGLPTVLHLKKRRFQCKNCLKVVVSQTSIVKKNCQISN
MVRQKIAQLLLEKQSMTEIAHRLAVSTSTVIRKLREFKFETDWTKLPKVMSWDEYSFKKS
KMSFIAQDFESKSILAILDGRTHAVIRNHFQRYQREVRELVEVITMDMYSPYYRLAKQLF
PKAKIVLDRFHIVQHLSRAMNRVRIQIMNQFDRKSLEYRALKRFWNPRFFVSRLGLNQST
GLIYYTRIASSSVRNDSISPRFECT
>orf00258
(SEQ ID NO: 111)
MGYSLKKSRTYCEQDPEKVNRFLKELNHLSYLTPIYIYETGVETYFYLEYDRALSRQLVS
LEEDIII
>orf00265
(SEQ ID NO: 112)
LREGCSIYDNLYPSRIVVGDETVEGRKIAELFLSISTHSTANIKNVMLVSPTEAEAIKLF
SNTFLALRVAFFNELDFFAERRSLNAEVVIKGVCLDPRIGNFYNNPSFEFGGYCLPKDTK
QLKKEFIEINAPVIEAIDISNTNRKQFIVKQILERKPKIVGIYKLGMKYNSDNYKESAIL
SIINELLIVGIKILVYEPNLNVSIDNVIFEKNFELFTKQSDLIVANRWDRGLEAYKDKVY
TRGIWIRD
>orf00268
(SEQ ID NO: 113)
MLNLQFAETMELTEAELETVYGGEFGNNAVIPAGAWGGLGTSWSITNFWKKYFNHDSSTV
NRRHY
>orf00272
(SEQ ID NO: 114)
MKIKEQTRKLAAGCSKHRFEVADRIDEVSSKHCFEVVDRTDEVSNHTYGKVKLTWFEESF
EEYK
>orf00338
(SEQ ID NO: 115)
LNTSYSFGKKDQFALEHCFCIKLSIFARAVTLFVSCIN
>orf00359
(SEQ ID NO: 116)
MLIGEGYRTFPVLIYTQFISEVGGNSAFAIMAIIIALAIFLIQKHIANRYSFSMNLLHPI
EPKKTTKGKMAAIYATVYGIIFISVLPQIYLIYTSFLKTSGMVFVKGYSPNSYKVAFNRM
GSAIFNTIRIPLIALVLVVLFTTFISYLAVRKRNLFTNLIDSLSMVPYIVPGTVLGIAFI
SSFNTGLFGSGFLMITGTAFILIMSLSVRRLPYTIRSSVASLQQIAPSIEEAAESLGSSR
LNTFAKITTPMMLSGIISGAILSWVTMISKLSTSILLYNVKTRTMTVAIYTEVLRGNYGV
AAALSTILTVLTVGSLLLFMKISKSNSITL
>orf00360
(SEQ ID NO: 117)
LIIIASMSTPFVGAYSWILLLGRNEVITKFLTNALYLPAIDIYGFKGIILVFTLQLFPLV
FLYVAGTMNSIDNSLLEAAESMGSFGFKPIVTVVLPLLVPILLAAPCLYL
>orf00362
(SEQ ID NO: 118)
LLSTTEFIGLSIRILSNLHESKILVGLLNQFFFWNLLLHKTKSNVVSDSQMWENSVVLEN
QPDIAFAGFHIIDFCIIEVKFSIFDTVETCNHTKKGRFPTS
>orf00380
(SEQ ID NO: 119)
VTAPTSITPLLVNTHERKSSQSLTSCLVYVVKTVLTSQHLIYSKLKLK
>orf00382
(SEQ ID NO: 120)
MITIKKQEIVKLEDVLHLYQAVGWTNYTHQPEMLEQALSHSLVIYLALDGDAVVGLIRLV
GDGFSSVLVQDLIVLPIYQRQGIGSALMKEALEDYKDAYQVQLVTEQTERTLGFYRSMGF
EILSTYNCIGMTWMNRKK
>orf00441
(SEQ ID NO: 121)
MGAFVLFLFQLTINRYKKKSFYWYKEVIESNGETLDN
>orf00465
(SEQ ID NO: 122)
VAIDKIAGITSEKDSRAHQIFRISPTCSRCFCNDELVKWVARTIFLQFTKRCCLRSGNIT
RSNSVTLDIGSTVFRRNVAGQHFQAPFSSSISANCFTSQFAHHRTNIDNLSMPFLYHRRN
NCL
>orf00466
(SEQ ID NO: 123)
LFDLLDHGLDTVLVCHVTDISMGFDANFTISFNPFIDQILIDIVKDNSSAGFSVGFGNSK
SNSIRSAGDESNFSF
>orf00478
(SEQ ID NO: 124)
MKSLARLLNIHVFISIFLFFALISGAVSHTVLLLLLLFLPALNKGLEKIQSKRIPVLNAA
LFFLLISFPQLLTNPVQWKFSIFLVVTIISSLAYFYNFYQVVKEVDQKQLI
>orf00480
(SEQ ID NO: 125)
LEAAGEIETEFQGWIVLVVFNHIDSLSRDTDILGEFELGNTQFLAKFFHTIHLVSFLIYV
VYI
>orf00485
(SEQ ID NO: 126)
MVDRTDEVSSKHGFEVVDKEKLMWFEEVFEECKKILVS
>orf00492
(SEQ ID NO: 127)
MEGVNHVDIIKVSCGSFISQVNWMMKGKIPNREGFKFSVARLDAIDLVVVHIGHTRCQFS
RTGSRSGYDNQVATDFDVVVFAHAFWGNDVIHIRRISFDWIMKIRINSVFLKLVAEGICS
GLTSVLCNDNGTNKNP
>orf00493
(SEQ ID NO: 128)
MFNVASINGNHNLNLLFQFLQELDFVVRFITRKDTSSVEIF
>orf00495
(SEQ ID NO: 129)
LFFHFLPLDSIIIKNWKLGNYGAKKEIKKIKQTLAQNFKKCYHIL
>orf00498
(SEQ ID NO: 130)
MQLTSVTAPTGTDNENIQKLLADIKSEYRFDGRPEFVLLGCLQESDCR
>orf00501
(SEQ ID NO: 131)
MIDIHSHIVFDVDDGPKSIEDSKALLREAYNQGVRMIVSTSHRRKGMFETPEEKIVTNFI
KVREIAKEVADDLVIAYGAEIYYTLDALEKLEKKKFLPLMIVVML
>orf00502
(SEQ ID NO: 132)
MHTSYREIHTRLSNILMLGITPVIAHIERYDALENNEKRVRELIDMGCYTQIDSYHVSKP
KFFGEKYKFMKKRARYFLERDLVHVVASDMHNLDSRPPYMQQAYDIIAKKYRAKKAKELF
VDNPRKIIMDQLI
>orf00503
(SEQ ID NO: 133)
MKEQNTLEIDVLQLFRALWKRKLVILLVAIITSSVAFAYSTFVIKPEFTSTTRIYVVNRN
QGEKSGLTNQDLQAGSYLVKDYREIILSQDVLEEVISDLKLDLTPKGLANKIKVTVPVDT
RIVSVSVNDRVPEEASRIANSLREVAAQKIISITRVSDVATLEEARPAISPSSPNIKRNT
LIGFLAGGIGTSVIVLLLELLDTHVKRPEDIEDTLQMTLLGVVPNLGKLK
>orf00505
(SEQ ID NO: 134)
MAKGHHIKLKVKDKIANVLTCNTISINSITSHSNSTQFMPFGMVLTQPLNIRRHPVFSNF
NLSSFYILTERTIQ
>orf00506
(SEQ ID NO: 135)
MKIAIAGSGYVGLSLAVLLAQHHEVKVIDVIKDKVESINNRKSPIKDEAIEKYLVEKELN
LEASLDPAHVYKDVEYAIIATPTNYDVDLNQFDTSSVEAAIKTCMEYNDTCTIVIKSTIP
EGYTKEVREKFNTDRIIFSPEFLRESKALYDNLYPSRIVVGTDLDDSELTKRAWQFADLL
KGGAIKEEVPILVVAFNEAEVAKLFSNTYLATRVAYFNEIDTYSEVKGLNPKTIIDIVCY
DPRIGSYYNNPSFGYGGYCLPKDTKQLKASFRDVPENLITAVVQSNKTRKDYIAGAILAK
QPSVVGIYRLIMKSDSDNFRSSAVKGVMERLDNYGKEIVIYEPTIECDTFMGYRVIKSLD
EFKNISDIVVANRMHDDLRDIQEKLYTRDLFGRE
>orf00507
(SEQ ID NO: 136)
MYTFILMLLDFFQNHDFHFFMLFFVFILIRWAVIYFHAVRYKSYSCSVSDEKLFSSVIIP
VVDEPLNLFESVLNRISRHKPSEIIVVINGPKNERLVKLCHDFNEKLENNMTPIQCYYTP
VPGKRNAIRVGLEHVDSQSDITVLVDSDTVWTPRTLSELLKPFVCDKKIGGVTTRQKILD
PERNLVTMFANLLEEIRAEGTMKAMSVTGKVGCLPGRTIAFRTEILRECIHEFMNETFMG
FHKEVSDDRSLTNLTLKKGYKTVMQDTSVVYTDAPTSWKKFIRQQLRWAEGSQYNNLKMT
PWMIRNAPLMFFIYFTDMILPMLLISFGVNIFLLKILNITTIVYTASWWEIILYVLLGMI
FSFGGRNFKAMSRMKWYYVFLIPVFIIVLSIIMCPIRLLGLMRCSDDLGWGTRNLTE
>orf00508
(SEQ ID NO: 137)
LIFEKEKCFLMLRKNLKYQIMTRAGTILAILFFIILGIIVEVLF
>orf00509
(SEQ ID NO: 138)
MKKVKKAVIPAAGLGTRFLPATKALAKEMLPIVDRPTIHFVIEEALRSGIEDILVVTGKS
KRSIEDYFDSTFELEYSLRKQGKMELLKSVNESTDIKVHFVRQSSPRGLGDAVLQAKSFV
GDDPFVVMLGDDLMDITDSTAVPLTRQLMDDYNATQASTIAVMPVRYEDVSSYGVISPRL
ESSNGLYSVDAFVEKPKPEEAPSNLAIIGRYLLTPEIFSILETQKPGAGNEIQLTDAIDT
LNKTQSVFAREFVGKRYDVGDKFNFMKTSIDYALQHPQIKESLKNYVIALGKQLEKLDDC
SSSGHL
>orf00510
(SEQ ID NO: 139)
MNCIESYQKWLNVPDLPAYLKDELLSMDDKTKEDAFYTNLEFGTAGMRGYIGAGTNRINI
YVVRQATEGLAKLVESKGETAKKAGVAIAYDSRHFSPEFAFESAQVLAAHGIKSYVFESL
RPTPELSFAVRHLGAFAGIMVTASHNPAPFNGYKVYGSDGGQMLPADADALTDYIRAIDN
PFAVALADLEEAKSTGLIEVIGETLDSAYLEEVKSVNINQDLIDQYGRDMKIVYTPLHGT
GEMLARRALAQAGFESVQVVEAQAKPAPDFSTVASPNEESQAAFALAEELGRQVDADVLV
ATDPDADRLGVEIRQADGSYWNLSGNQIGALIAKYILEAHKQAGTLPKNAALAKSIVSTE
LVTKIAESYGATMFNVLTGFKFIAEKIQEFEEKHNHTYMFGFEEG
>orf00520
(SEQ ID NO: 140)
MNKGLFEKRCKYSIRKFSLGVASVMIGAAFFGTSPVLADSVQSGSTANLPADLATALATA
KENDGRDFEAPKVGEDQGSPEVTDGPKTEEELLALEKEKPAEEKPKEDKPAAAKPETPKT
VTPEWQTVEKKEQQGTVTIREEKGVRYNQLSSTAQNDNAGKPALFEKKGLTVDANGNATV
DLTFKEDSEKGKSRFGVFLKFKDTNNNVFVGYDKDGWFWEYKSPTTSTWYRGSRVAAPET
GSTNRLSITLKSDGQLNASNNDVNLFDTVTLPAAVNDHLKNEKKILLKAGSYDDERTVVS
VKTDNQERVKTEDTPAQKETGPVVDDSKVTYDTIQSKVLKAVIDQAFPRVKEYTLNGHTL
PGQVQQFNQVFINNHRITPEVTYKKINETTAEYLMKLRDDAHLINAEMTVRLQVVDNQLH
FDVTKIVNHNQVTPGQKIDDERKLLSSISFLGNALVSVSSDQTGAKFDGATMSNNTHVSG
DDHIDVTNPMKDLAKGYMYGFVSTDKLAAGVWSNSQNSYGGGSNDWTRLTAYKETVGNAN
YVGIHSSEWQWEKAYKGIVFPEYTKELPSAKVVITEDANADKKVDWQDGAIAYRSIMNNP
QGWEKVKDITAYRIAMNFGSQAQNPFLMTLDGIKKINLHTDGLGQGVLLKGYGSEGHDSG
HLNYADIGKRIGGVEDFKTLIEKAKKYGAHLGIHVNASETYPESKYFNEKILRKNPDGSY
SYGWNWLDQGINIDAAYDLAHGRLARWEDLKKKLGDGLDFIYVDVWGNGQSGDNGAWATH
VLAKEINKQGWRFAIEWGHGGEYDSTFHHWAADLTYGGYTNKGINSAITRFIRNHQKDAW
VGDYRSYGGAANYPLLGGYSMKDFEGWQGRSDYNGYVTNLFAHDVMTKYFQHFTVSKWEN
GTPVTMTDNGSTYKWTPEMRVELVDADNNKVVVTRKSNDVNSPQYRERTVTLNGRVIQDG
SAYLTPWNWDANGKKLSTEKEKMYYFNTQAGATTWTLPSDWAKSKVYLYKLTDQGKTEEQ
ELTVKDGKITLDLLANQPYVLYRSKQTNPEMSWSEGMHIYDQGFNSGTLKHWTISGDASK
AEIVKSQGANDMLRIQGNKEKVSLTQKLTGLKPNTKYAVYVGVDNRSNAKASITVNTGEK
EVTTYTNKSLALNYVKAYAHNTRRDNATVNDTSYFQNMYAFFTTGSDVSNVTLTLSREAG
DQATYFDEIRTFENNSSMYGDKHDTGKGTFKQDFENVAQGIFPFVVGGVEGVEDNRTHLS
EKHYPYTQRGWNGKKVDDVIEGNWSLKTNGLVSRRNLVYQTIPQNFRFEAGKTYRVTFEY
EVGSDNTYAFVVGKGEFQSGRRGTQASNLEMHELPNTWTDSKKAKKATFLVTGAETGDTW
VGIYSTGNASNTRGDSGGNANFRGYNDFMMDNLQIEEITLTGKILTENALKNYLPTVAMT
NYTKESMDALKEAVFNLSQADDDISVEEARAEIAKIEALKNALVQKKTSLVADDFASLTA
PAQAQEGLANAFDGNLSSLWHTSWGGGDVGKPATMVLKEPTEITGLRYVPRGSGSNGNLR
DVKLVVTDESGKEHTFTTTDWPDNNKSKDIDFGKTIKAKKIVLTGTKTYGDGGDKYQSAA
ELIFTRPQVAETPLDLSGYEAALAKAQKLTDKDNQEEVASVQASMKYATDNHLLTERMVE
YFADYLNQLKDSATKPDAPTVEKPEFKLSSLASDQGKTPDYKQEIDRPETPEQILPATGE
SQSDTALFLAGVSLALSALFVVKTKKD
>orf00523
(SEQ ID NO: 141)
LQIAQESSQDTDGINPPVVEEAMVFDRNDCLNQICGNIISLGIDAAFRTQVSNELIFIVV
DFTRSCCN
>orf00525
(SEQ ID NO: 142)
MLNLMWMKIFHRNRTFLFCFLDFKVDVISIINARIVRR
>orf00526
(SEQ ID NO: 143)
MYNSQALRQIVVVGSIDHLFKRHSSICEIFGLRKRCLSFLW
>orf00537
(SEQ ID NO: 144)
MKLLKKTMQAGLTVIFFGLLATNTVFADNSEGWQFVQENGRTYYKKGDLKETYWRVIDGK
YYYFDSLSGEMVVGWQYIPFPSKGSTIGPYPNGMRLEGFPNSEWYYFDKNGVLQEFVGWK
TLEIKTKDSVGRKYGEKREDSEDKEEKRYYTNYYFNQNHSLETGWLYDQSNWYYLAKTEI
NGENYLGGERRAGWINDDLTWYYLDPTTGIMQTGWQYLGNKWYYLRSSGAMATGWYQEGT
TWYYLDQPNGDMKTGWQNLGNKWYYLRSSGAMATGWYQEGTTWYYLDQPNGDMKTGWQNL
GNKWYYLRSSGAMATGWYQDGSTWYYLNAGNGDMKTGWFQVNGNWYYAYSSGALAVNTTV
DGYSVNYNGEWVR
>orf00541
(SEQ ID NO: 145)
MTTGWFQVNGRWYYAYSSGALAVNTTVDGYFVNYNGEWVQ
>orf00551
(SEQ ID NO: 146)
MSLADLLEELEAAKDSKKARSMEAYMRHQFSFLGIAVPERNKLYKNIFQKRKKQRLSIGI
LQTLAGKRILENTNMWLLTI
>orf00552
(SEQ ID NO: 147)
MEKILLHNLNQTEFFINKAIGWTLRDYSKTNPTWVTCFIEKNKERMAELSIKEASKYL
>orf00555
(SEQ ID NO: 148)
VLEILKEYLLLEEGDYIFTNNGSPLMITCFNYFLKNSFRKSEIKKDDFVLTAHVFRYSHI
SLLAELEVPITAIMDRVDYTNETKILSVYTHVTEKMKSNITEKLDKLVLEND
>orf00580
(SEQ ID NO: 149)
LVIFKNCSHCTSNCKSRTVQGMKEFHLAICFITVTDLSTTGLEIFXXXFHSLIN
>orf00581
(SEQ ID NO: 150)
MNXXARGFSFVCKDFEVAAHFASSDIGSNLIDMAPRNLDKLFDIDIWVKFQSLISDKEFP
NFPSDREVGSACRTEEYCFVK
>orf00587
(SEQ ID NO: 151)
LQKPLFQILQKLVKISQAFIHDSNFFLAHAINNFXXHSFIN
>orf00590
(SEQ ID NO: 152)
VGFSLLLLLIFCLFCLLFNVSNQVTNCFQVIFCFDLDIKSIFDF
>orf00614
(SEQ ID NO: 153)
VNIDSSEFYISHITDGIFDSFLDSNRYLRNFYSVLKVEIDICCEFFVHVFKINATAE
>orf00615
(SEQ ID NO: 154)
VNTLYLCSSNSNDFFKYTWGDNDFAKLFFNSHRMTSF
>orf00625
(SEQ ID NO: 155)
LFSPRPGGDNGKGTTFLNFFLNIHKFSFFDSLFFLFQ
>orf00627
(SEQ ID NO: 156)
VKEEKKAIVLGADNAYMDKVETTLKSLCVHHYNLKFYVFNDDLPREWFQLMEKRLETLNS
EIVNV
>orf00643
(SEQ ID NO: 157)
LAQISILHFDFLSIDKHSHTVFNTLRKSLQTTLALSATSKQCFEQLAASFLVCSLIFIEY
KV
>orf00652
(SEQ ID NO: 158)
MSLITHRRFISSKVIRQKFVDNQIDLKYYIWRYSHALCGIDVAKNKHDVTALNVSGKTVL
KPLTFSNNKAGFELLDLSLRQLNQDYLIALEDTGHYAFNLLNFLHEQGYKVYTYNPLLIK
KFAKSLLLRKTKTDKKDAHGIALKLLSDPNREQFQHDNRQVELKILARHIHRLKKKQSDW
KVQYTRCLDIIFPELDKIVGKHSEYTYQLLTCYPNPQKRLEAGFDKLIEIKRLTASKIQD
ILSVAPRSIGTTSPAREFEIIENIKHYKRLIDKAETCVNDLMAEFNSVITTVTGIGNRLG
AVILAEIQNIHAFDNPAQLQAFAGLDSSIYQSGQIDLAGRMVKRGSPHLR
>orf00654
(SEQ ID NO: 159)
MDTKSSCLITTGRNDSPSTCLPRVASNNDRFSSEFRIIPDFHCSKKGIHVNMDDFS
>orf00657
(SEQ ID NO: 160)
MDKMKPVFQALNKELIQENLTLTIICVGGYVLEYHGLRATQDVDAFYDQNQKINEIIARV
GKQFNLNTHEELWLNNHVANMNKQPPLSLCESLYSFENLTVLVVPIEYVLGMKMMSIREQ
DLKDIGAIIKYKNFHSPFDTFKYLKDMGFDTIDLSVLLEGFSYAYGMDWLEKFFKENQDK
LREFY
>orf00663
(SEQ ID NO: 161)
MIPLYRTDNDITKFFTKIRNGHLAKTAGGLDDKFHEANASTSKAFDRQGVGEVNDIRDSA
GSQELRINDKRKTENILFLEIRVRIFRVPHPNDSFFSSHFLG
>orf00664
(SEQ ID NO: 162)
VLSQGDKDITILDAGLLKNGKIGPVTKDTNDIKATDNMIENSFVLLNQQNIMLFCNQGAT
EGKTNFSPSDKDNFHNKTYFFMM
>orf00669
(SEQ ID NO: 163)
MKIKEQTRKLAAGCSKQCFEIVDRTDEVSSKHGFEVVDETDEVSSKHGFEIVDETDEVSN
HTYGKAKLTWFEEIFEEYKMMGKAGQLVFFDVYRLVRQVS
>orf00698
(SEQ ID NO: 164)
MEKFNFKNNIGQENKLLQIEIYKFTNFCKLQNYTSVNIFSKDIFEAIVN
>orf00701
(SEQ ID NO: 165)
VRIKSIYWNFGQNKPEKSFRYIDTSSIDRKKNIINYKNLQYLSPEQAPSRARKLVSQNSV
LFSTVRPYLKNIAVVRELKEYLIASTAFIVLDTLLNETYLKYYLLSDNFINRVNNKSTGT
SYPAINDYNFNLLLIPLPPLSEQQRIVEAIESALEKVDEYAESYNRLEQLDKEFPDKLKK
SILQYAMQGKLVEQDPNDESVEVLLEKIRAEKQKLFEEGKIKKKDLDISIVSQGDDNSYY
GNIPMNWVVIKIKDIFSINTGLSYKKGDLSINKGVRIIRGGNIKPLEFSLLDNDYYIDTQ
FISSEQVYLKHNQLITPVSTSLEHIGKFARIDKDYDGVVAGGFIFQLTPFESSEIISKFL
LFNLSSPLFYKQLKAITKLSGQALYNIPKTTLSELLIPLAPFEEQELITQKVEKLFEKVN
QLWK
>orf00720
(SEQ ID NO: 166)
MIRKVNHNIFKHRSVVIFTLTNSYFCKFFINDISISFHSHQHFCWIIIQIK
>orf00724
(SEQ ID NO: 167)
LDNIHIVLDSLNAVSGIQDFICDGLAIFCNQITSGCSSCK
>orf00735
(SEQ ID NO: 168)
MNIAWILLYALVINGLEIVIFFKVDGIGLTFDRIFKAFLLKFLLGIIFTTFQFLAVSKYL
SYFIEPLFGIGLSFLLLRGLPKKILIFYGLFPMILVELFYRGVSYFVLPFLGQGIVDGDG
NPIFLLIMIFVCFIVLVFLKWLDYDFTRLRREFLDTGFQKSLTKINWAMGAYYLVMQSLS
YLEYEQGIQSTTVRHLILVFYLLFFMGGIKKLDTYLKEKLQEELNQEQTLRYRDMERYSR
HIEELYKEIRSFRHDYTNLLTSLRLGIEEEDMEQIKEIYDSVLRDSSQKLQDNKYDLGRL
VNIRDRALKSLLAGKFIKAREKNIVFNVEVPEEIQVEGMSLLDFLTIVSILCDNAIEASA
EASQPHVSIAFLKNGAQETFIIENSIKEEGIDISEIFSFGASSKGEERGVGLYTVMKIVE
SHPNTNLNTTCQNQVFRQVLTVIHAE
>orf00737
(SEQ ID NO: 169)
MISQEDILKACEVAEIRQDIERMPMGYQTQLSDGAGLSGGQKQRIALARALLTKSPVLIL
DEATSGLDVLTEKKVIDNLMSLTDKTILFVAHRLSIAERTNRVIVLDQGKIIEVGIHQEL
MQAQGFYHHLFNK
>orf00738
(SEQ ID NO: 170)
MSSKISIGQLITFNTLLSYFTTPMENIINLQTKLQSAKVANNRLNEVYLVESEFQVQENP
VHSHFLMGDIEFDDLSYKYGFGRDTLTDINLTIKQGDKVSLVGVSGSGKTTLAKMIVNFF
EPYKGHISINHQDIKNIDKKSLAPSY
>orf00740
(SEQ ID NO: 171)
MKSTLGIISVGLVITYILQQVMSFSRDYLLTVLSQRLSIDVILSYIRHIFELPMSFFATR
RTGEIISRFTDANSIIDALASTILSLFLDVSILILVEGVLLAQNPNLFLLSLISIPIYMF
IIFSFMKPFEKMNHDVMQSNSMVSSAIIEDINGIETIKSLTSEENRYQNIDSEFVDYLEK
SFKLSKYSILQTSLKQGNKISSEYPYPMVWRSISHVE
>orf00742
(SEQ ID NO: 172)
MTSYKRTFVPQIDARDCGVAALASIAKFYGSDFSLAHLRELAKTNKEGTTALGIVKAADE
MGFETRPVQADKTLFDMSDVPYPFIVHVNKEGKLQHYYVVYQTKKDYLIIGDPDPSVKIT
KMSKERFFYEWTGVAIFLATKPSYQPHKDKKNGLLSKLPSSDFQTKISHCLHCSLKLIGH
YYQYRWFLLSPRNLG
>orf00767
(SEQ ID NO: 173)
MGLIKTLAKIYGNYFLTVQGVKVMKTIKKDDHVVVGLGKLFIADKLMDTARWLIKPEDKK
>orf00768
(SEQ ID NO: 174)
MKFFWGLLAIIFIKPIIGIVKFFWMIISFAVQLLFYKIVFKILDWLFKLI
>orf00776
(SEQ ID NO: 175)
MHSQTFQFLLMTDKTSLLHRKHRSFIRNIHSKFLILFDLLCGILSRNDSNHNPIS
>orf00779
(SEQ ID NO: 176)
MARTELPDKIETERLVLRVRTVADAEDIFDYASLPEIAYPAGFPPVKTLEDEIYYLEHIL
PERNQKENLPAGYGIVVKGTDKIVGSVDFNHRHEDDVLEIGYTLHPDYWGRGYVPEAARA
LIDLAFKDLGLHKIELTCFGYNLQSKRVAEKLGFTLETRIRDRKDVQGNRCDSLIYGLLK
SEWEE
>orf00781
(SEQ ID NO: 177)
MSDVKEEVSSLSEKQLRQIDVEYAELNDSDIIERLAYLEINNNEKRIVISDIEPTKEIMS
VSDQIFEIQKNFQKIKNMFELFISDVSDFLSIKNKLESKELEIEEADVNRFMIHLLSSGK
LFVDFNENQIKQKYSKDSEEFDCIHGFASYQYDINFTYRFCHSLRNYSQHTDLPINEVKA
VSPDDETVIIDFYIDLDYLLNSNFKWKKLKGELIKLNQETSKIDAIALVKEYFNALTELY
GNYNKLFLKLNHNTLVDIKSKLESLKLKHSRYYISKISKYDLKYNPGNYTMSPLAAFAEI
EEIYIELSKIGLVKIVNKSN
>orf00785
(SEQ ID NO: 178)
MSKHPHYELLNLIGYGLAKFDKLFIKEFQCSSKSEFYRYVVSLGIAETTGVVKNRMDLFD
PYFDNNRKGWWQKAEVYRFRKDLIDMMFGNEDVHSYAEIVKMLLASEGKKTGITIVEKPI
VRTKFKRLQETGMEAENYFILHFDKEEKFQGGQLTDARLYGDGYDFQVDVQEYSYLAEVK
GIRKSKGRVRLTAKEFEKVKEFQSDFILSLVTNLDDIPKLVLIDNPLKHFEFKKNIIKNE
IIEYRSVEDLY
>orf00787
(SEQ ID NO: 179)
LSTCWNGKFCHICVALFHCFRAFKLALNEILCLLTNVSFIFVSVAF
>orf00788
(SEQ ID NO: 180)
LLRKQEREYLRAENAILKKLRELRLKEEKEKEERQKLFKN
>orf00809
(SEQ ID NO: 181)
LKHLFCHFNPLWIDEIIRLAYKDQDTKDVKSKVKIGN
>orf00856
(SEQ ID NO: 182)
MKEIAFDAFYQLYQNDQLSLVDVREVDEFAALHLEGAHNLPLSQLADSYD
>orf00859
(SEQ ID NO: 183)
MVSNHKIACFQLFDKVGIFSLLDELNSLANKAHINLLNCF
>orf00871
(SEQ ID NO: 184)
MDFFFMNEVKEQVLFRDNHSEHIFWIEGVSDFMIKVNTALW
>orf00878
(SEQ ID NO: 185)
MKINEQTRKLAAGCSKHCFEVVDRTDEVSNHTHGKATLTWFEEIF
>orf00885
(SEQ ID NO: 186)
MKINEQTRKLAAGCSKHSFEVVDKTDEVSSKHGFEVVDETDEVSSKHGFEVVDETDEVSN
HIYGKATLTKFELDFRRV
>orf00890
(SEQ ID NO: 187)
MEELVTLDCLFIDGTKIEANANKYSFVWKKTTEKFSAKLQEQIQVYFQEEITPLLIKYAM
FDKKQKRGYKQSAKNLANWHYNDKEDSYIHPDGWCYRFHHIKYQKTQTDFQQEIKVYYAD
EPESAPQKGLYMNERYQNLKAKECQALLSPQDRQIFAQRKIDVEPVFGQIKACLGYKRCN
LRGKRQVRIDMGLVLMANNLLKHSEMK
>orf00892
(SEQ ID NO: 188)
MHIHYNTNQTTLPLEISSFLPQDHLVFTIEKVVNTLEDCHFHAFYHAFDRPSYHLKMLVS
TLLFAYSQGIFSGRKIEKWKS
>orf00894
(SEQ ID NO: 767)
LRLWVIFVIMKVMKSYNTLNDYYRKLFGEKTFKVPIDAGFDCPNRDGTVAHGGSTFCTVS
GSGDAIVAPDAPIREQFYKEIDFMHRKWPDVQKYLVYFQNFTNTHEKVEVIRERYEQAIN
EPGVVGINIGTRPDCLPDETIEYLAELSECMHVTVELGLQTTYEATSDLINRVHSYEL
>orf00896
(SEQ ID NO: 189)
VETVKRLRKYPKIEIVSHLINGLPGETHEMMVENVRRCVTDNDIQGIKLHLLHLMTNTRM
QRDYHEGRLQLMSQDEYVRVICDQLEIIPKHIVIHRITGDAPRDMLIGPMWSLKKWEVLN
SIEMEMRRRGSVQGCKAVKQEFENEKTT
>orf00908
(SEQ ID NO: 190)
VQVCVFTNFCFFHCFSSLANCRLFNLRGICLPCISYQ
>orf00915
(SEQ ID NO: 191)
VFKKDRFSIRKIKGVVGSVFLGSLLMAPSVVDAATYHYVNKEIISQEAKDLIQTGKPDRN
EVVYGLVYQKDQLPQTGTEASVLTAFGLLTVGSLLLIYKRKKIASVFLVGAMGLVVLPSA
GAVDPVATLALASREGVVEMDGYRYVGYLSGDILKTLGLDTVLEETSAKPGEVTVVEVET
PQSTTNQEQARTENQVVETEEAPKEEAPKTEESPKEEPKSEVKPTDDTLPKVEEGKEDSA
EPSPVEEVGGEVESKPEEKVAVKPESQPSDKPAEESKVEPPVEQAKVPEQPVQPTQAEQP
STPKESSQQENPKEDRGAEETPKQEDEQPAEAPEIKVEEPVESKEETVNQPVEQPKVETP
AVEKQTEPTEEPKVEVTSIPQTTRYEEDLTKEHGTREVVKEGKNGSRTVTTPYILNATDG
TTTEGTSTTDEAEMEKEVVRVGTKPKEKLAPVLSLTSVTDNAMLRSARLTYHLENTDSVD
VKKIHAEIKNGDKVVKTIDLSKERLSDAVDGLELYKDYKIVTSMTYDRGNGEETSTLEET
PLRLDLKKVELKNIGSTNLVKVNEDGTEVASDFLTSKPVDVQNYYLKVTSRDNKVFRLTV
EKIEEVTEEGQPLYKVTAKAPNLIQHTDATKMQDEYVYYIEKTRATDGDIYYNFNDLVNA
MNKNKTGTFKLGADLNATGVPTPAKSYVTGDFRGTLTSVDGEHYTIHNTSRPLFNNIIGG
TIKDINLGNVNIHMPWANNVASLANIIKGGTTIENVKVTGNVLGKDWVSGFIDKIDSGGT
LRNVAFIGNVTSVGTGGSFLTGIVGENWKGLVEQAYVDANIRGKKAKAAGIAYWSQNGGD
NYAVGRYGAIKKSVVKGSIDVEKPIEVGGAVGSLNYLGYIEDTVAMMKVKNGEIFYGSHD
IDTDPYYTGERVNRNFIVDGVSEGKSSYKYSKQQNRIKSVSQEEADKKIKELAITADKYA
ITEPIVNKLNALTTRDNEYRTTQDYKADRELAYRNIEKLQPFYNKEWIVDQGNKVPSNSK
LLTTEVLSVTGMKDGQFVTDLSEIDKIMIHYADGTKEEMNVTAVADSKVKQVREYDVTDL
GVVYTPNMVDKNRDQLIADVKAKLSSVELISPEVRALMDKRGKAEENTEGRQNGYIRDLF
LEESFAEVKAGLGKLVKALVENEDYQLNSDEAAMRALIKKVEDNKAKIMMGLAYLNQYYS
FKYAELSIKDIMMFKPDFYGKNVNVLDFLIKIGSSERNVKGDRTLEAYRETIGGTIGINE
LNGFLHYNMKLFTNHIDINDWFKKAIEKNAYVVEQPSTNPAFANKKYRLYEGINNGQHGR
MILPLLNLKNAHLFMISTYNTISFSSFEKYGKDTDEKREKFKSEINKRAKEQVNYLDFWS
RLATDNVRDKLLKSQNVVPTPVWDNHNSPNGWASRHGHIDGKPDYAPIREFFGRINKYHG
YKYGYGAYAYIFAAPQPMDAVYFVMTDLISDFGTSAFTHETTHINDRMAYYGGHWHREGT
DLEAFAQGMLQTPSVSNPNGEYGALGLNMAYERQNDGNQWYNPNPNKLKSRAEIDHYMKN
YNEALMMLDYLEAESVLPKLKGNNDRWFKKMDKQMRKDGQPHQFDKIRDLNNEEKKIQLA
SIEDLVDNNFMTKHGAPGNGTYNPSDFSSAYVNMNMMTGVYGGNSSDGAPGAASFKHNTF
RMWGYFGYENGFIGYASNKYKAEANKAGQTLSDKYIINKVSGGTFNTLEAWKKEWFKQIK
TKAQKGFTAIEIDGKTIDSYEKLKDLFDKAVEEDLKGTGTDKTVKLKEKVYKQLLKNTDG
FSGDLFTAPQA
>orf00933
(SEQ ID NO: 192)
VGDRIFIAFLQKLGLLDNLTGIREKLHPITGQGDSLGIADKDLNAHFIFQISHCIGETWL
SDKELLGCLIHGASFDDFDNIM
>orf00941
(SEQ ID NO: 193)
VSRWDGHSDKGEAPAGKTSYAWIWTKWGEQVAFYCDYD
>orf00955
(SEQ ID NO: 194)
MKLFKPLLTVLALAFALIFITACSSGGNAGPSSGKTTAKARTIDENKKSGELRIAVFGDK
KPFGYVDNDGSYQGYDIELGNQLAQDLGVKVKYISVDAANRAEYLISNKVDITLANFTVT
DERKKQVDFALPYMKVSLGVVSPKTGLITDVKQLEGKTLIVTKGTTAETYFEKNHPEIKL
QKYDQYSDSYQALLDGRGDAFSTDNTEVLAWALENKGFEVGITSLGDPDTIAAAVQKGNQ
ELLDFINKDIEKLGKENFFHKAYEKTLHPTYGDAAKADDLVVEGGKVD
>orf00988
(SEQ ID NO: 195)
MDTPDENGYVADDYRITYLEAHIKAMRDAIYQDGVDLLGYTTWGCIDPVSAGTGEMNKRY
GFIYVDRDNVGNGALKRSKKKSFYWYKDVIDSNGASIG
>orf01015
(SEQ ID NO: 196)
MTEPDFWNDNIAAQKTSQELNELKNTYNTFHKMEELQDEVEILLDFLAEDESVHDELVAQ
LAELDKIMTSYEMTLLLSEPYDHNNAILEIHPGSGGTEAQDWGDMLLRMYTRYGNAKGFK
VEVLDYQAGDEAGIKSVTLSFEGPNAYGLLKSEMGVHRLVRISPFDSAKRRHTSFTSVEV
MPELDDTIEVEIREDDIKMDTFRSGGAGGQNVNKVSTGVRLTHIPTGIVVQSTVDRTQYG
NRDRAMKMLQAKLYQMEQEKKAAEVDSLKGEKKEITWGSQIRSYVFTPYTMVKDHRTSFE
VAQVDKVMDGDLDGFIDAYLKWRIS
>orf01045
(SEQ ID NO: 197)
MQVIKRNGEIAEFNPDKIYQAILKAAQTVYVLTDDLRQNLAQVTKKVVLDLQEAKVERAT
ISMIQSMVEHRLLGAGYITIAEHYISYRLQRDLERSGYGDHIAVHLHFEQIR
>orf01068
(SEQ ID NO: 198)
MELFKTWKKNMVLYGLKSQIGTVYRNNDRTTSFYDVGNFLYLAGELDSRFWEDFVRKYGL
DYKIIISENTNWQDFLHRKVGLNSFTRYSFKDKANFQVEFLNNLVTHLEEGYNIVPIDNH
IYNCFSTEEWSQDLQGDFESYQDFVLKGGFGFVILKNNELIAGISSGLVYRKAVEVEVAT
RPNEQGNGFAKKLGAAMILESLNRDMFPLWDAHNEASKKVAEFLGYELSEPYEAFELEEI
LI
>orf01090
(SEQ ID NO: 199)
LGSDRKALHKPVYLFWCESFDILFCTWSSQFSVLKAFI
>orf01110
(SEQ ID NO: 200)
MKVINQTLLEKVIIERSRSSHKGDYGRLLLLGGTYPYGGAIIMAALAAVKSGAGLVTVGT
DRENIPALHSHLPEPMAFSLQDQQLLKEQLEKAEVILLGPGLRDDASGENLVKQVFVNLS
QNQILIVDGGALTILARTSLSFPSSQLILTPHQKEWEKLSGITIEKQKEDATASVLTSFP
QGTILVEKGPATRIWEVGQSDYYQLQVGGPYQATGGMGDTLAGMIAGFVGQFRQASLYER
VAVATHLHSAIAQELSQENYVVLPTEISRYLPKIMKIICQQDRVSKDKLV
>orf01114
(SEQ ID NO: 201)
VLASNRKFIFFFFRIGILILKNIKSFNQFLALFHKIPSHDGSRKSLSWSDGKSLKXXFIH
>orf01121
(SEQ ID NO: 202)
VLDSKEELKESENDAPKLETPLREEPRLAPQTLLEASEVLENKREESKVGITEPAQDSPI
LAPVEETKEEAVTEKPTNTRSLTAEDLVKISKGELHLENDLIDESFYGEKALDLEGDDYQ
DGIKNKDGKDYLGYNSHPLLADSDGDGLADGEDDNKKEWYVTDRDSLLFMELAYRDDDYI
EKILDHKNLFPSLYLDRQEHKLMHNELAPFWKMKKAYYTDSGLDAFLFETKSDLPYLKDG
TVHMLAIRGTRVNDAKDLSADFVLLGGNKLAQADDIRKVVGELAKDISITKLYMTGHSLG
GYLAQIAAVEDYQKYPDFYNHVLRKVTTFSAPKVITSRTVWDAKNGF
>orf01148
(SEQ ID NO: 203)
MGRNPKTRPEERTELERLQSENEYLRAENAILKKLRELRLKEEKEKEERQKLFKN
>orf01150
(SEQ ID NO: 204)
LSTCWNGKFCHICVALFHCFRAFKLALNEILCLLTNVSFIFVSVAF
>orf01165
(SEQ ID NO: 205)
VVLSTSAILVACGKTDKEADAPTTFSYVYAVDPASLGYSIATRTSRTDVIGNVIDGLMEN
DKYGNVAPSQKDYDLNSTGWAPSYQDPASYLNIMDPKSGSAMKHLGITKGKDKDVVAKPG
LDKYKKLLEDAVSETTDLEKRYEKYAKAQAWSTDSSLLMPTASSGGSPVVSNVVPFSKPY
SQVGIKGEPYIFKGMKLQKDIVTTKEYNEVFKKWQKEKLESNSKYQKELEKYIK
>orf01169
(SEQ ID NO: 206)
LNFDFFIFLAHFIPLFTFSILQENPKTSKKKLYIRLL
>orf01176
(SEQ ID NO: 207)
MGFSMKLIHDLDTHTTHSTAKMLYNVKAIKNDFSIRE
>orf01191
(SEQ ID NO: 208)
LIRIIRNIYRSGEGNTSVFQSFIDQINSNQFCYGSNFDRLRCILLIENFTSICLNSNRMF
SGNGKILSNSSRSTP
>orf01202
(SEQ ID NO: 209)
MIYFDNSATTKPYPEALETYMQVASKILGNPSSLHRLGDQATRILDASRHQIADLIGKKT
DEIFLTSGGTEGDNRVLQGVAFEKAQFGKHIIVSPLPHSAVLE
>orf01223
(SEQ ID NO: 210)
MKIKEQTRKLAAGCSKHCFEVVDKTDEVSSKHGFEVVDETDEVSNHTYGKATLTRIEEIF
EEYKSS
>orf01233
(SEQ ID NO: 211)
LVYAPFSFNILLDYITFDFKILLFSVFLAINRFHNDFIQFLL
>orf01236
(SEQ ID NO: 212)
MSGYSGLSFFEVALAEFLDIVSAVYLEDADGIIVNLWGILDK
>orf01246
(SEQ ID NO: 213)
MEGVAKGRIGRKKNNGIDNRCCHKKRNGRVTWNLFFQKTIDDGDDSTFTRREKHTDEGPK
KDSPPTISREKMINLVRCDINFNQP
>orf01249
(SEQ ID NO: 214)
VDKTDEVSSKHCFEVVDKTDEVSSKHCFEVVDKTDEVSSKHCFEVVDRTDEVSSKHCFEV
VDRTDEVSNRTTVRRS
>orf01251
(SEQ ID NO: 215)
MDYGLYHPCPIVTPSQSSSIVANPASKLISASEELIPDAIAVDFVEVNCY
>orf01254
(SEQ ID NO: 216)
MKKLFQEKFSKKPSHKEIERVQLGCAMMQATFHLMGY
>orf01261
(SEQ ID NO: 217)
VVIGVASATTNIWIIFLSGFAAILAGAFSMAGGEYVSVSTPKDTEEAAVSREKLLLDQDR
GLAKKSLYAAYIQNGECKTSAQLLTNKIFLKNPLKALVEEKYGIEYEEFTNPWHAAISSF
VAFFLRSLPPMLSVTIFPSEYRIPATVLIVGVALLLTGYTSARLGKDPTRTAMIRNLAIG
LLTMGVTFLLEQLFSI
>orf01269
(SEQ ID NO: 218)
MLYVGIDVAKNKHDVTALNVPGKLFLNHSLFQIIKLVLNS
>orf01296
(SEQ ID NO: 219)
MDFEYFYNREAERFNFLKVPEILVDREEFRGLSAEAIILYSILLKQTGMSFKNNWIDKEG
RVFIYFTVEEIMKRRNISKPTAIKTLDELDVKKGIGLIERVRLGLGKPNIIYVKDFMSIF
QVKENDLQKSKNLTSEVKDFNLRSKENELQEVKNLDSNYIENNKSKYSKREYSFGENGLG
TFQNVFLAAEDISDLQIIMNSQLENYIRLPAKLES
>orf01304
(SEQ ID NO: 220)
LLHIRVCKTFDRIPYCMLALFLSKSIGLTILLHKVKTVIFIDDQSNDKTCKICIHISFFR
IKLSQQCQLSFSVYF
>orf01309
(SEQ ID NO: 221)
MTQEDALIVISHIKVLSIVPNRCLKPLDKTFSLYNWIFLSQKYILLQANFLKISRVQLQ
>orf01313
(SEQ ID NO: 222)
VTTHDEPVYEKHGVLHYAVANIPGAVARTSTIALTNVTLPYIEALAGKGFAQAISEDEGL
RQGVTTYQGYLTSLPVAQGLNRDYTDINDLV
>orf01314
(SEQ ID NO: 223)
VFFIDGFIVRCHTVSCFDNATLVNSNVNDTEPGRICLTISSVTNSGAFAPGMRMAPITTS
ASLILASMLNELDIRV
>orf01316
(SEQ ID NO: 224)
MLIGIPKEIKNNENRVALTPAGVHSLVSRGHRVLIETNAGLGSGFTDADYQKQGAEIVAT
AGEAWAAELVVKVKEPLSSEYGYLRDDLLLFTYLHMAAAPELADAMLAAKTTGIAYETVR
DNQGQLPLLVPMSEVAGRMAV
>orf01334
(SEQ ID NO: 225)
LIRILGNSFISIDKAHEQAEEYGHSFEREMGFLAVHGLFTY
>orf01383
(SEQ ID NO: 226)
VILFFIRFRVSWLTYFIMSLIFIRFTILVCLIFTNMVASVTKAIILMADVRLDVRHST
>orf01387
(SEQ ID NO: 227)
MIAMRSYITLICNLNNNLFCLNSFFLTNLVWSQIFSLLSVFITVYI
>orf01388
(SEQ ID NO: 228)
MFLFLAIDFIFYSYIFCMSLIFKVNIILSIYLNNISSLNLTDDILIFRLIVGGFH
>orf01390
(SEQ ID NO: 229)
VIPRGDHNHYIKVQTKGYEAALKNKIPSLQSNYQPGTFDEKAVLAKVDQLLADSRSIYKD
KPIEQRQIELALGQFTESLKKIKVS
>orf01419
(SEQ ID NO: 230)
MARLEPAKIAKIVLGILLYIIDLIKSSFVLPIPKAAKKSLILISFVPSFNDKNIVIRRPR
QITKIMPRFICFLFRIFACIS
>orf01436
(SEQ ID NO: 231)
MELSAIYHRPESEYAYLYKDKKLHIRIRTKKGDIESINLHYGDPFIFMEEFYQDTKEMVK
ITSGTLFDHWQVEVSVDFARIQYLFELRDTEGQNILYGDKGCVENSLENLHAIGNGFKLP
YLHEIDACKVPDWVSNTVWYQIFPERFANGNALLNPEGTLDWDSSVTPKSDDFFGGDLQG
IIDHMDYLQDLGITGLYLCPIFESTSNHKYNTTDYFEIDRHFGDKETFRELVDQAHHRGM
KVMLDAVFNHIGSQSLQWKNVVKNGEQSAYKDWFHIQQFPVTTEKLVNKRDLPYHVFGFE
DYMPKLNTANPEVKNYLLKVATYWIEEFNIDAWRLDVANEIDHQFWKDFRKAVLAKNPDL
YILGEVWHTSQPWLNGDEFHAVMNYPLSDSIKDYFLRGIKKTDQFIDEINGEFMYYKQQI
SEVMFNLLDSHDTERILWTANEDVQLVKSALAFLFLQKGTPCIYYGTELALTGGPDPDCR
RCMPWERVSSDNDMLNFMKRLIKIRKYASVIISHGKYSLQEIKSDLVALEWKYEGRILKA
IFNQSTEDYLLEKEAVALASNCQELENQLVISPDGFVIF
>orf01442
(SEQ ID NO: 232)
MGQEIKLIRKQFRSTRQEEKQIKEMMREQKVDSFSEFLRQNLLKKNYQDRIFESWFSLWQ
SQKFEQISRDVYEVLVVARENHQVTQEHVSILLTCVQELIAEVNQVQPLSREFREKYMG
>orf01443
(SEQ ID NO: 233)
MVYRYRTNLKKVFLTDPELHQLNERIAKSNCQNFSVYARKVLLNPNMSFVTINTDTYDQL
VFELRRIGNNINQIARAINQSHLISQDQLQELSKGVGELIKEVDKEFQVEVKRLKEFHGS
Y
>orf01444
(SEQ ID NO: 234)
MVVTKHFATHGKKYRRRLIKYILNLDKTDNLKLVSDFGMSNYLDFPSHAEMVEMYNVNFT
NNDKLYESRNDRQEKHQQTIHAHHIIQSFSPEDNLTPEEINRIGYETMMELTGGRFKFIV
ATHTDKDHVHNHILINAIDRNSDKKLIWNYALERNLRMISDRISKMTGAKIIEKRYSYRD
YKKYKESSHKFELKQRLYFLLQQSKSFDDFLEKAKQLHVQIDFSQKHSRFLMTDRTMIKP
IRGRQLSKRDLYDEEFFRTHFAKQEIESRLEFLLNRVNSLEDLITKAKELNLTIDLKQKN
VTFILEENNQKISLGHQKISDKKLYDVKFFQDYFKNKEVVASEGLENLQEQYHAFQEERD
KDKVSTEEIEEAFKTFKEK
>orf01446
(SEQ ID NO: 235)
VELAENQIEKLVDKGVYIKVSFGVKQSGLIFIPNYQLDIMEEENHKKYKVYIRETSSYFV
YNKENMDNNCFIKGRTLIRQLSNDSQKLPYRRPTLKSLQEKISEINLMIELSNTNKQYQE
IKDELVLEIAEIDMKLEETQEKIATLNKMAEVLINLKSEDHETRKLARYDFSKLNLTEST
SLENVNEEIRVLQENLDYYLYEFEKRAIRLEIFVSTLNMEKDVFVIDKF
>orf01447
(SEQ ID NO: 236)
MAIIKKIVVLYGGLSEEREVSENSAKEISKSLLTLGYDVISIDLSQDCSYEIGEIEKSER
GLNKQKVEIGSGIIDVCRKVDIVFLATHGGIGENGKLQAIFDVEKIDYTGNSFLSTAISM
DKKLSKIVASSVGIKCASNLMVDRIQANDFPIVIKPIRSGSSKGIKIFQNKKQFDIYYKE
HPELGSVFVEKYIKGREFSVGILGETVLPVIEIKVKNGFYDYNNKYTVGAAEEVVPAKIS
EKLTHTLQKSAYKIHKALGFNVYSRTDFIVDEKGDVYFIESNSLPGMTKTSLLPQEAKAA
GIDFPNLCERIIELSREIRSQ
>orf01449
(SEQ ID NO: 237)
MKIINGICRYIFDSKGFATIEVEIFLDSGDTGIGAAPRGSTTGHYDIQYNEYYPRGNNFS
PIPDGNIEFFNENILPRIINREVEDIEDITELDKHLFDIPEIENYGNVAIACSYAVWEAF
SKNKKSPLWKLFFEPGSASKGKVKHLVNIIDGKPDSLLAGFEFLLVSEKEITFQSLLEIS
NIKNELMIKFKNQGFYTSISNQGALIINTDDFYIILDSLLETLKLYKNRYDIGLDLAMTD
RYDSSLGIYKVPWCVSQQQTVTEIMDTYCDWGVKYPLVYLEDPFSDEDLDSWRKFQLIKP
LKLQVFGDDFYATNLERISQFKDCADGIVIKPNQVGSVSKTLEVMEYAEKSGISMAFSQR
TAETENNIISHLAMSVTSSYLKAGGLDRLDRIAKYNEVLRNG
>orf01450
(SEQ ID NO: 238)
MDNGKISTDGALVNNKNIEVNISSASIRYGISVFEAPKMFLLGSDIYIFRFQDYFNRLKT
SCKFLNIELPLDYNKLLYDIKKFIEFVPWQESYALRINVFCPYESELLGEVECALTLSYL
DIGIRSKSGVSSKVIKRSNLIRTSNNNLYMIKSPSHYINARKELYSDIEFDDILYVNEKE
NICELSRSNIFLIKDRTVYTPDLGSGILDGITRKLIIDLCQEHNILIEIKELNYSKLSDF
DSAFCVGTTNGITPIRNIDKNIHFDTNNLLLAEIVNYYKEVFSKKGVEKYKEWFVKL
>orf01451
(SEQ ID NO: 239)
MIKDVNYFNERAQIIRRTINDMIFGAESGHFGASLSSVDFINVIYENYVFPENAEFILSK
GHAAPALYAKLIESGVLDKDFIYGFREYRSLLTGHPNHRIPTLKFGLGSLGQGPSIGVGM
AWVNKRKKSDKKIFVMLGDGELNEGQVWEAFYTCRNLNLQNLVFIIDRNFLQLDGKCEDV
ANFPNLAQKISSFLGTNPIEVNGNSYDEILNVLDNIDYSQTNVIISNTTKGKGIEFMEGK
TEFHSYNILSSEKEVLYKRAMECLGGDKMA
>orf01452
(SEQ ID NO: 240)
MRKTFIDELINKNIEEQNIVVLTGDVGRSTYASKFKEIFPENYLNLGICEANIVGISAGI
AGTLEFVPFVMLFSKHLILRALEQINDSILMNKKKVILVGGYSGYSASKEGETHQLLNDI
SILSSFPDISIYCPYDQSSIQTAINESINNDYSSYIRINKNKILNDVVSRYISQGNKSII
ISMGYLGSLLSKKYMEDSDIREFADFILVSKIKPIDWEYWENFLRKYETVYIIEENTLTG
GLGEQFKNYFFGLGINIISFGIKPHFGETGDYETLLTNEGLSPDKIFEKIRRINHVQSSE
KSIWR
>orf01453
(SEQ ID NO: 241)
MYNLVKRVFGDDNFVKSDGIYLYTDDNKEIIDSCSNSMNVNLGYGVLEIEKVIHEQIRKI
NFMHTGKGTTYESLMLANRLHDIIKPYGNYKVYFATSGSDCIEAALRISVLYQQKNKRNQ
ESNTRFATFEGSYHGSTLGALSVSGHRKFQKLYNGYIGNCLTIPTRWNLEWEMDFTDITA
FVLDSMITNPIGSEMIDRDYLNYLVQICKESGVITIIDEIATSIGRLGCFFGFEDSGISP
DIVCISKGLGVGYANIGAVIVKSNIIDSINSADILGHTYNASPIDCKIAMTVLDYIEEHG
IFEHVNNLSNYIEYRLKVLKNKLPCISKVSGKGFMWSIHFRNNVNASKVFKQCYNNGLLL
LYLEYEEYNHMTFCPPLITSKNQIDSMLDILEKSITEVLHGY
>orf01455
(SEQ ID NO: 242)
MDIKKLGFIIITNETFAFNLVEEVVEKLSILDVNRAIIKLLPKVDETTLIKHYREHLINW
NGKEGKSQVPSLGWPAVRNRFSSSVVLILLEGNKQSLSDEILQIKGNTYPERCRQNQIRI
KGFNPTYNLMHSSDSAEEALKEAELYFSKKELESFFCGEIGITFEEILNFILELDSIQKG
GIRSNSDLENKVNKMCYWKQRIFDLFSSKTKEESRKISEELNFIKSNRSPEDVILYLENE
SILWSKLERDLYLSHLIIEEISGE
>orf01456
(SEQ ID NO: 243)
MVKIYEETLRDGIQSSELSMLNTEDKKKVIKNFDNAGIEGCCIGFINSSKKENDEINELF
QFIITNKLKIVPAVLCRLLIEDFKEIKNIVQCNLFNQLKVYTYIAVSPIRMKVENWSEEI
IIEKIIDFLNYCQMENAKITIAFEDASRADKSFLKKLIEIINNYPLVKSVVIADTVGTCN
YNSTRDLVYFFRKNLFLSKTIEWHGHNDLGLAVSNALSAIESGADIVHTCTLGIGERCGN
TSTEQLIINLIVSECGRRATYKLRYLYDVAKILENKSQFPISPKTPCFGMDSFFTCAGTH
ASSLYKSYLKNDNNFSIIFSPYDSNIFGREVTVGINSQSGRSSIEYISRKYGLELSQENI
TSILGLVKQNDLFFREEEFVDYVKQKRVERLDYK
>orf01457
(SEQ ID NO: 244)
MLNKKEWKDLTISKSENDSIISCLYNWGFELPKSKDYTESLTWYGYNNVSYKYCEKECLT
LGELSLLFPQLNDLFWENQKDNIEGRRNFYKNCKNLLGKFNIYSVRDILEISDRDLQFAL
TNRVGSKEFENSIVSLMYINKLSDDNKAENTLEKKLYFSINNEDSIIPYYENTLGMTAGN
YYFTASHRTFFSSTFPDFFKIDVPYKISNSVRNLTRNELRIRASEIIKSELANSENPILK
IFDDYAYVRIGKRNMVLRNKFFNGFIAPLFAFFQQRIFDNGKSFFTHFYDLRKNTSQNIP
DYLLLNILFPLIDCFEFFLNVSIKYKRLIFPYDFHMQNIMISIYDNQIGFIFQDFDMSKE
LTVTSFYEQLDEFFEKFVVTNILESINELKLDEKKYIMREMKKIINTKIIIKAESYFGFP
SPSEVIFPDFMKQYLRENQNVKERTIKYRSYLR
>orf01458
(SEQ ID NO: 245)
LFIDILKKKKMKIIEAHDALSAYIIDQTKYSADKGDIFEYDGIWISSLCDSIIRGKPDME
VVNITDRLQTIDDILCVTEKHIIVDIDSGGTLAQTASLIEKITLRKIAGIVIEDKIGQKY
NSLFGEKNTQLQDSTESFERKIRVAIKSRKNSKTAIIARIESLVLGKSLEDLLYRGNCYI
KSGAEALVIHALNSRLDDLYQALKYFKRYFPEIPLIVIPTDFPYVCAEELFESGADLIIY
ANHLLRSIIKPMEYIAKSILIDGYSNGVENKLLPISEILNYIPLLEEIDEK
>orf01459
(SEQ ID NO: 246)
MKSKDLISSLKLNGVTRFFGVPDTFLYPITSQLANDELIIVPNEGNAVSMAFGYSITTSR
MPVIFLQNSGLGNILDPVQSLVGQAVYNHPMLYIIGFRGGTDDAPQHSECGKVTKKLLEI
SQFDIYEKDYFTNALDTDIRRIIDNIKKKNKSAAILVDKEFFSDIVNIKNYQISDYNDIL
NKICAVLEKEKDSIVLTSTGYITRHMENVKDTYKKNFVHIPMPGSLGQTISFGAGICMGT
SINNKKKKIYIIDGDGSLFMHMGSLALFDYYNLDVTYILLNNYKHLSVGGENTLASSCNF
FKLAESMNFDNIYSSDNLEVVDFEEKLNQQGKNFIEIICSNEVTYSLLERMDFNFEEIKQ
FNMEN
>orf01460
(SEQ ID NO: 247)
MPDRYVEYSQNDYDNIMKLDTLFISQLDSKYKVEYHKLPSLISGEVLKRCGYFSTMPNQL
SKVDIIDVSQLESLGNNRELEEIKYSSSENYFLTPAACLHFYPMLEHKEVKECIYSSLVD
VFRYENGNFTMGTRQWEFTVREFLAIGNPKFVEEFLEDLKEKFLTIALRFDSTAKIQNAC
DNFYPTNLNKVKQKFQKYNNLKFELIVHISEKEVSVASFNYHNNHFSKEFNEDSNDTIVT
GCVGLGIDRWISLIKESEKDYKL
>orf01461
(SEQ ID NO: 248)
MRKQIISLFHAYLNENTQDWVEIDENFLLYQHLDMFYYYLCKKNNIEPPLMEKFEVRKKV
IESRNRQYIKVARDLNAIFEKQSIQVAFLKGIQTSEKYYEEPWIRYYSDLDILVAREMIP
GVEKLFYQLGYVFGHLKDNGEIHHATREEILYQKLFTHEIYNLVKKENDNVFINVDINFL
FSWKGLSDSEIEFNDIKNDIIYDSKIKINTLDKVMNFIHICCHLYNEARYFALNRSFLGG
DPREIQLSRVFEIALILKDLKQEEFNSVVYSSRKLNCDNKVFASIGVTKQLLELNLSILD
NYTEELKVKDEFNSYIDKDQKVKYWPISIEDRVFDLKLKVKTCDKIFIN
>orf01462
(SEQ ID NO: 249)
LKYDSEIKNVIKDYIDFEFDINEIDNNVALTDYGVDSLKYISIILALEDYFKIEIPDNYL
VFSKSNTIKKLNSIIEELL
>orf01463
(SEQ ID NO: 250)
MKYINSSLKPVDNYYSLEEVTCFDRLLGIFLNSINNSYCDLFYMINNFYRCYKVDDSKNE
YDFEREMHILRNFFCIESEKINFSDEYVLTEFIVKNINKYGSVFVPINLKEIYYSAYYKE
QDWPHLFLINGYDKEKELFYVIDATQIYSDILTEQNFCITFEILERAYKSYFHQTILNKE
KEYIFVVTTVVNELTETEIFHETFKYLFNQSSISSRELEIVYKILTNRDFTLLANLKNIT
KKKKLFFTIFFEKLRVYELISNKELLYLSRTVETILEEWTIFINRCIKNILKNDTKLVNY
DFFVLEEKKIFDFFSKQASRYKERLFEIISSNSNRYNEVFECIQNSGKIITITEDNSRKF
RFSFIGDKIYNSWFNDDSPKVRINQDLNYLIVKINVIHKEKDSKFVAGLYCFIDDNLYYF
GLDSNYFINLDLMGKTPEIFRKRLETSEVYLKISRQEDSCKFSYSVDGITYFYATSLDIR
SCHFSCGIGCKTYSKPTPLCIDFEDLLIG
>orf01464
(SEQ ID NO: 251)
MAMIEVSCLRKDFVKIIKEPGLKGAIRSFIHPEKQIFEAVKDLSFEVPKGQILGFIGANG
AGKSTTIKMLTGILKPTSGFCRINGKIPQENRQDYVKDIGVVFGQRTQLWWDLALQETYS
VLKEIYDVPDAVFQKRMDFLNDVLDLKEFIKDPVRTLSLGQRMRADIAASLLHNPKVLFL
DEPTIGLDVSVKDNIRRAITQINQEEETTILLTTHDLSDIEQLCDRIFMIDKGQEIFDGT
VNQLKETFGKMKTLTFELRPGQNHVVSQFVGISDIHVTRKELLLNIQYDSSRYQTADIIQ
KTLSDFAIRDLKMTDVNIEDIIRRFYRKEL
>orf01466
(SEQ ID NO: 252)
MVKLWKRYKLFISAGMQELITYRVNFFLYRIGDVMGAFVAFYLWRAVFSSSHQSLIRGFS
LSDMTFYIIMSFVTNLLTKSDSSFMIGEEVKDGSIIMRLLRPVHFAASYLFTEIGFRWLV
FVSVGLPFLIIIIGLKLLSGQPILQISLMTLTYLLSLILAFLINFFFNICFGFSAFVFKN
LWGSNLLKNSLVAFMSGSLIPLSFFPKIIADILYFLPFSSLIYTPVMIIVGKYNISQMIQ
AVLLQLFWLLVMIALSQIIWKRVQSHITIQGG
>orf01467
(SEQ ID NO: 253)
LLVGISLLSATVTSLTWTWTKVFIFLISIPFATLIYTSLKIATASIAFWTKQSGAVIYIF
YMFNDFAKYPVVIYNSFLRWLISFIIPFAFTAYYPASYFLKDKDGLFNIGGLILISLIFF
TISLKLWNKDLDAYESSGS
>orf01468
(SEQ ID NO: 254)
MDEVFKFYCTNIIRVIFIKWLFCLKSMNHTNLTLVTTTFKQFNSTSLGNHHAMV
>orf01469
(SEQ ID NO: 255)
MQAFDGSTEFIHNGKGEKDSSKYQINGAGRCEVPVYVPKTDWDWIIYPQGIYD
>orf01471
(SEQ ID NO: 256)
MRVKRDYPNYKIYITENGLGYKDEFVDNTVYDDGRIDYDYVKKHLEVI
>orf01472
(SEQ ID NO: 257)
MEWIINIIRGIRYIGREAGVTFVEPSQTSIKSFDITIL
>orf01473
(SEQ ID NO: 258)
VRHYKDYISQRSDWELAGIYADEGISGTQVGKRQDFQRLINDCVNGEIDYIVTKAIARFA
RNTLDTLKYVRMLNDMQIGVYFEEENIDILTMDGELLLTILSSVAQQEVENTSAHVKKGL
KMKMERGELVGFQGCLGYDYDVETKQISINKKESKIVRYIFERYLEGIGGKVIARELDEL
GYKSPRGLEHWNDTTVLGIIKNEKYKGDILIGKTFTVDPISKRRLSNFGEEDKYYIKDNH
EPIISK
>orf01474
(SEQ ID NO: 259)
MYAFSSMLECGFCGSILSRRSCHCRSDYRKVVWHCVTSIKKGKKFCKHSKGLEKLAIEGA
FMEAYRQLYHSNENLMTDLLETIESELNDNSLNKELKRITNKLRTLLKKEENLVNLRLEG
KISDTIYDEKYNEISSEKEFLAEEKVNIETTLKSEIDVKKRLTEFKHLLSSQKMLTEFDR
AVFESIVEKIIVGGVNSNGEIDPAMLTIIFKTGETQNKDGKQFKSKRKNAKLETDKLCPQ
NSDEDKKLYSQGTDNTRGVCSVAGSILVSQ
>orf01475
(SEQ ID NO: 260)
MGGNPPIKKYSIVDKIVLSTKIKRIIIFTVFRENWEPYMKKYTEVFQSQFPNLNIDYLLL
DTEQIDLDSYLDADLIIIGGGNTEKYIATYVNQEFKNYIDHMLNKGAKVIGFSAGALLLG
EKVYVSPNDNSDHQIKIKDGLGLFSQFLISVYYDSWNDKANKDRAEELVNVPIIPLNDHS
CLVLDKLGNIIEKID
>orf01476
(SEQ ID NO: 261)
MDFSSKIAINTGWSDDKKYCVTDQNQQKYFLRVSDKEKLDSKKFEFDMMGKVASLGVPMC
KPISIELCDDEVHSLHEWIDGRDAIDSILTYSENQQYTYGVEAGKILRKIHTIPATEVCE
DWEIFFNLKIDDKISNEMIW
>orf01514
(SEQ ID NO: 262)
LCSALKNSYDIELIKVLSNKAHLYLPIETVTPQTVSTS
>orf01537
(SEQ ID NO: 263)
LVKNPFIEIERIERTWLTAHLYRKFDKYFHKTRPPIKVFEHLIGGLFIMKTFT
>orf01538
(SEQ ID NO: 264)
MIKIYFTKFSENHNPFCKIFEIIFTSLIFQSILNKNKKNPLHQGETNVV
>orf01545
(SEQ ID NO: 265)
MSQVKGLCVLDVDGTLILEEVIDLLGREAGHEAEISQITSRAMRGELVFESSLRKRVSLL
EGLPILVFDNVFNSIHLSLNVPEFISILQKNGILVGLVSGGFTPIVGEISKIPWYCLFHC
QPA
>orf01546
(SEQ ID NO: 266)
MLKSAELGIAFCSKEMLKKEIPHHVDKRDFLEVLPLIDCLE
>orf01551
(SEQ ID NO: 267)
MFGNWFFKAFVCSLERLAQDRTMNWFSCIGNKNTVAFVPILIGCFA
>orf01565
(SEQ ID NO: 268)
MEKYFGEKQERFSFRKLSVGLVSATISSLFFMSVLASSSVDAQETAGVHYKYVADSELSS
EEKKQLVYDIPTYVENDDETYYLVYKLNSQNQLAELPNTGSKNERQALVAGASLAALGIL
IFAVSKKKVKNKTVLHLVLVAGIGNGVLVSVHALENHLLLNYNTDYELTSGEKLPLPKEI
SGYTYIGYIKEGKTTSDFEVSNQEKSAATPTKQQKVDYNVTPNFVDHPSTVQAIQEQTPV
SSTKPTEVQVVEKPFSTKLINPRKEEKQSSDSQEQLAEHKNLETKKEEKISPKEKTGVNT
LNPQDEVLSGQLNKPELLYREETIETKIDFQEEIQENPDLAEGTVRVNQEGKLGKKVEIV
RIFSVNKEEVSREIVSTSTTAPSPRIVEKGTKKTQVIKEQPETGVEHKDVQSGAIVEPAI
QPELPEAVVSDKGVPEVQPALSEAVVTDKGETEVQPESSDTVVSDKGEPKQVAPLPEYKG
NIEQVKPETPVEKTKEQGPEKTEEVPVKPTEETPVNPNEGTTEGTSIQGAENPVQPAEES
TTNSEKVSPDTSSENTGEVSNKPSDSKPPVEESNQPENSGNTTSENGQTEPEPSNGNSTE
NVSTKSNTSNSNGNEEIKQENELDPDKKVEDPEKTLELRNVSDLELYSLSNGTYKQHISL
EQVPSNPNSYFVKVKSSSFKDVYLPVASISEERKNDKILYKITAKVEKLQQEIESRYKDN
FTFYLAKKGTEETTNFTSFSNLVKAINQNLSGTYHLGASLNANEVELSTDDKSYIKGTFT
GQLIGEKDGKHYAIYNLKKPLFESLRGATIEKLSLKNVSISGKDDIGSLANEAQNNTKIK
QVHVDGVLAGERGIGGLLAKADQSSITESSFKGRIVNTYETTASYNIGGLVGHLTGSKAS
LTKSKATVVISSNTNSSDQTVGGIAGLVDKDAHIQNSYSEGDINNSQRFGKVAGIAGNLW
DRESNSENHAGRLTNVLSDVNVTNGNAISGYHYNGMKITDAFSNKANKVFNVTLEKDEVV
SKESFEERGTMLDASQIASKKAEINLLTPPIVKPLSTSGKKDSDFSKIAHYQANRALVYK
NIEKLLPFYNKATIVKYGNLVKENSILYQKELLSAVMMKDDQVITDIISNKQTANKLLLH
YKDHSSEKFDLRYQADFAKLAEYSLGDTGLLYTPNQFLYDQDSIINQVLPELQQVAYDSE
AIRKTLGISPEVKQTELYMEDQFTKTKQDLANSLKKLLSADAGLAGDNPVTRGYLVDKIK
NNKEALLLGLTYLERWYNFSYGQVNVKNLVMYHLDFFGKGNTSPLDTLIELGKSGFNNLL
AKNNVDTYAISLASHHGTTDLFSTLENYRKVFLPDKTNNDWFKSQTKAYIVEEKSNIEEV
KTKQGLVGTKYSIGVYDRITSDSWKYRNMVLPLLTLPERSVFVISTISSLGFGAYDRYRN
KEHQANGDLNSFVEKSAHETAERQRDHYDYWYRILDEKGREKLYRNILLYDAYKFGTDHT
EGKATEVANFDNPNPAMKHFFGPVGNKVGHNGHGAYATGDAVYYMGYRMLDKDGAITYTH
EMTHDSDQDIYLGGYGRRSGLGPEFFAKGLLQAPDQPSDATITINSILKHKTSDSTEGQR
LQVLDPTTRFNDAADLQNYVHNMFDVVYMLEYLEGQSIVKQLDAYQKMTALRKIENKYVK
DPADGNDVYATNVVKNLTEDEAKKLTSFDSLIDNNILSAREYKAGTYERNGYFTIKLFAP
IFSALSSEKGTPGDLMGRRIAYELLAAKGFKDGMVPYISNQYEEDAKQQGQTINLYGKER
GLVTDELVLKKVFDGKYKTWAEFKTAMYQERVDQFGNLKQVTFKDPTKRWPSYGTKTINN
VDELQKLMDEAVLQDATGTRWSNYNPEIDSAVHKLKRAIFKAYLAQTNDFRSSIFENKK
>orf01580
(SEQ ID NO: 269)
VLGGRANSVTSCTTNSHWNLTFTTKHVTCFSSLVDDIVHGNNREVHEGHIDDWTKSCHGC
SCCSSRDGSFRNRTVTDTFWTKFFKHSNRSTEVSSEDTDVFSHQEHIFIATHFLRHSKDN
GVTEGHCFCFHFISFSLVCVNIFKG
>orf01581
(SEQ ID NO: 270)
MDMFYIGHFLDIRRDTVTVVNAIENDWQVPDRSHVHCFVENTFIGRTISKEADNDFTGIL
HLLTEGCTDSDPHTTTYDTIGTKVPSIKVSDMHRSTFPFTGSSVFTKDFSHHSVEVNPFS
NSLPVSTVV
>orf01602
(SEQ ID NO: 271)
MKINKKYLVGSAAALILSVCSYELGLYQARTVKENNRVSYIDGKQATQKTENLTPDEVSK
REGINAEQIVIKITDQGYVTSHGDHYHYYNGKVPYDAIFSEELLMKDPNYKLKDEDIVNE
VKGGYVIKVDGKYYVYLKDAAHADNVRTKEEINRQKQEHSQHREGGTSTNDGAVAFARSQ
GRYTTDDGYIFNASDIIEDTGDAYIVPHGDHYHYIPKNELSASELAAAEAFLSGRENLSN
LRTYRRQNSDNTPRTNWVPSVSNPGTTNTNTSNNSNTNSQASQSNDIDSLLKQLYKLPLS
QRHVESDGLIFDPAQITSRTARGVAVPHGNHYHFIPYEQMSELEKRIARIIPLRYRSNHW
VPDSRPEQPSPQSTPEPSPSLQPAPNPQPAPSNPIDEKLVKEAVRKVGDGYVFEENGVSR
YIPAKDLSAETAAGIDSKLAKQESLSHKLGAKKTDLPSSDREFYNKAYDLLARIHQDLLD
NKGRQVDFEALDNLLERLKDVSSDKVKLVDDILAFLAPIRHPERLGKPNAQITYTDDEIQ
VAKLAGKYTTEDGYIFDPRDITSDEGDAYVTPHMTHSHWIKKDSLSEAERAAAQAYAKEK
GLTPPSTDHQDSGNTEAKGAEAIYNRVKAAKKVPLDRMPYNLQYTVEVKNGSLIIPHYDH
YHNIKFEWFDEGLYEAPKGYTLEDLLATVKYYVEHPNERPHSDNGFGNASDHVQRNKNGQ
ADTNQTEKPQTEKPEEETPREEKPQSEKPESPKPTEEPEESPEESEEPQVETEKVEEKLR
EAEDLLGKIQDPIIKSNAKETLTGLKNNLLFGTQDNNTIMAEAEKLLALLKESK
>orf01625
(SEQ ID NO: 272)
MDESFDDIAHEQFTSNLTTKTDNVGVQLFFSIKGCCHITNQGRANTWNFIYSVVDTNTST
TDTYSKISLAASYSFPYFFTKDWVVSPCMVICTKVNDFISF
>orf01629
(SEQ ID NO: 273)
MLDFQDRSPWLEGQKEIDLSYDLFSTDAVTLDELQSRTIALRSLKHDKGLKVHFAEFPNL
IIWSTLNKGPFITFEPWSGLSTFLEEGDHLEDKKNVCLLEANQVEELGFEIEVL
>orf01634
(SEQ ID NO: 274)
MKIKEQTRKLAAGCSKHSFEVVDETDEVSNHTYGKATLTWFEEIFEEYKN
>orf01643
(SEQ ID NO: 275)
LIESQVFSSLQVCCLNLCHLKFQHFDTCLVFLLVFLDFQNLLAHFPIGIKTRLIGFFQVP
KSGITKFIQHLDMQLGTH
>orf01644
(SEQ ID NO: 276)
MVMLTMNIYKMLPNSSQNRQINHLTIYTADTTTILQDFPTDDNFIT
>orf01645
(SEQ ID NO: 277)
MTNNICRRTSSQHHIHGINDNRLPCTRFTSQDSHPLFKIEGNSLNNGKVFYRNFK
>orf01656
(SEQ ID NO: 278)
MPHTRDNWQTRFKNSSYHNFFVKGPEILNRTTSTTNNEQIQIVPLISTRNISSNFLRSPF
TLNLGRIKKDVNTWESPADGRDNISNNGSTTAGYYTNSLRKLGQSLLEAFLKQAFFCQFF
LKLFKLNRKRPNPIRLSFFNDDGVATTWFIDLYTPNHIDLHSFFQVKP
>orf01660
(SEQ ID NO: 279)
LAIIRNRTCSLKLINGHLTFWTLHFLTSTRILIELATINLNCRIHRGNLGNRPSQASNRF
INKLFIQGRQNRGFCDHFPTSILSRRGIAQSNFPLIDLTLVLHKLDHACRLANRNRQNTH
HIRIQGSTMTNFLGSQNLTQFKNRIMRGHSCFFF
>orf01667
(SEQ ID NO: 280)
MFALRKPGNIYTRITNPTTAALEGGVEALATASGMTAVTYTILAIAHAGDHVVAASTIYG
GTFNLLKEPLPRYGITTTFVDIDNLEEVEAAIKDNTKLVLIETLGNPLINIPDLEKLAEI
AHKHQIPLVSDNTFATPYLINVFSHGVDIAIHSATKFIGGHGTTIGGIIVDSGSFDWTAS
GKFPQFVDEGPSCHNLSYTRDVGAAAFIIAVRVQLLRDTGAALSPFNAFLLLQRLETLSL
RVERHVQNAETIVDFLVNHPKVEKVNYPKLADSPYHALAEKYLPKGVGSIFTFHVKGGEA
EARKVIDNLEIFSDLANVADAKSLVVHPATTTHGQLSDKDLEAAGVTPNQIRLSIGLENV
EDLIEDLRLALEKI
>orf01668
(SEQ ID NO: 281)
MTRDFKFETLQLHAVQVVAPATKSRAVPIYQTTFFVFDDT
>orf01672
(SEQ ID NO: 282)
MSQKKNNKKKNKRKNLLTNILAGFLILLSLALIFNTQIRNIFIVWNTNKYQVSQVSKEKL
EENQDTEGNFDFDSVKAISSEAVLTSQWDAQKLPVIGGIAIPELEMNLPIFKGLDNVNLF
YGAGTMKREQVMGEGNYSLASHHIFGVDNANKMLFSPLDNAKNGMKIYLTDKNKVYTYEI
REVKRVTPDRVDEVDDRDGVNEITLVTCEDLAATERIIVKGDLKETKDYSQTSNEILTAF
NQPYKQFY
>orf01679
(SEQ ID NO: 283)
MRWNIGCHPNRDTSCSINQKVWKTRWQDQGFPFIGIIVINEINCIFVDITKHFQSNLAHT
CLGITLSGSTISIHGTKIPMTIYKHVTVAPPLSHTDHGFINRGIPVWVIFTHDIPCNTSR
FFMGFVWGHTQFIHSVENATVNRF
>orf01683
(SEQ ID NO: 284)
LKKKWFFADYYDTTIILLALISVILVLLGFAEMIDLDNPPYSIIDLVIWGVFVIDYSWRF
FITKRKWRFILENIFDLLAILPLNAIFTVFRLGRIFRLVKLTKLLKLTRLLRIIGLTGKL
ERKISRFLRTNGLIYILYVNIFIILVGSSILSVVEEKSFSDSLWWALVTVTTVGYGDIVP
VSLLGKWLAVLLMLVGIGTIGMLTSALTNFFVKDNPDEQIKLDKLKDELSS
>orf01693
(SEQ ID NO: 285)
VVDFKQTRQDPHDITIYSWLRQVKSNTGNGSCCVRSNPFQAGNSFIGIWKLATKVSHNLL
GCSLHIANSRIITQALPSFQ
>orf01698
(SEQ ID NO: 286)
LINSQLIPLVQVVVNQGRKGIVGSCNSMHISSKVEVDVFHWQNLCIPTTSSTTLDPHDWT
KRRFADSNHGFLANLVQGIRKTNGKRRLSFTCRCWVDGSNQDQFTDWIALNCTNFIKAEF
SLVLSVQLQIVVRNTKFLYNINNWLQLNTLCDFNICFHSKFL
>orf01711
(SEQ ID NO: 287)
MLFIIGHLNFPTAGSFIDSTLHRLGNRVCIHDDMAFTVTSSTSNSLDESTFVAKETFLVS
IENSYEAHFRNVNSFTEQVNSDQDIKDTQAQVTDNLRPFQGLDIRVHVLDLDTHFLEVVG
QILCHFLGQSCDKGTLIFFNAGIDFTQEVINLSHSRTDFHLWIQESRWTNDLLNHCLGLF
IFIVTRCR
>orf01712
(SEQ ID NO: 288)
MNVTLKLLPTERTIVQSRRQTETIINQHFFTRTVSIVHALDLPYGHMTLVNHNQEIIWEE
VEKRIRRLSFAPSIHVARIIFNPIGIAHLTQHFDIILCPLFQTLGFKQFTFLFKDS
>orf01713
(SEQ ID NO: 289)
MIHFSQHLTCQSLNFTNTVNFVSKKFYSKGMFISGSWENLYHIPTNAKSSALEINIIAFK
LNIDQVIQEFITRNL
>orf01714
(SEQ ID NO: 290)
VAKLVNLVIDRTILLNIGIARRDIGLWLVIIIVGYEILNCIFREKFLKLPIELTSQSFIV
GNNQSWFIDFRNDLTHSIRLPCSSRPHQNLSFFSPLNVIHQLLDSLGLIS
>orf01734
(SEQ ID NO: 291)
MNITQTDFLAVNLVFAISTTIDMAFHPDFLTCILDKSVMIIQSHNYRSIIERFATFGSSK
DDIRHLAPTETLDTRLPQSPTQTFCNIGLSRSIGSNNCRHTLVKNDLGLISKRLEPLNFD
FL
>orf01736
(SEQ ID NO: 292)
MGFIVCNHLKLACFNLRNHDLIDKFLDLGHILIQKKGTKKGFKGITKNGVTIATTRFFFP
FTQLDKLVKLAITRKTS
>orf01748
(SEQ ID NO: 293)
LFTCFSKLDNKTASTTYISHKFFTAIPVCFEFFKGFWFPRKDTTKKNIFIPMFLVECFNF
WVELR
>orf01753
(SEQ ID NO: 294)
VVRELTGEIYFGDHILEERKARDINDYSYEEVERIIRKAFEIARNRRKIVTSIDKQNVLA
TSKLWRKVAEEVAQDFSDVTLEHQLVDSAAMLMITNPAKFDVIVTENLFGDILSDESSVL
SGTLGVMPSASHSENGPSLYEPIHGSAPDIAGQGIANPISMILSVVMMLRDSFGRYEDTE
RIKRAVETSLAAGILTRDIGGQASTKEMMEAIIARL
>orf01754
(SEQ ID NO: 295)
MAKKIVALVGDGIGPEIMEAGLEVLEALAEKTGFDYEIDRRPFGGADIDAAGPPLPDETL
KASREADAILLVAIGSPQYDGVAVRPEQGLMALRKNSIFTLIFVL
>orf01758
(SEQ ID NO: 296)
LANIESHCNFFQSSIFSSLPNTIDSLFNTSCTILDSSKAICHCHSEVIMTVRRIDDLTIR
LDILNQVFEDGTTFLWCGKSYIF
>orf01768
(SEQ ID NO: 297)
MPRNRFSFTVRVTREKNFISFFSFFFQVIDKRAFSSDIDILRFIIIFNIDGHTGFLQITD
MPDTG
>orf01772
(SEQ ID NO: 298)
MKIKAQTRKLATGCSKHCFEVVDKTDEVSSKYCFEVADGS
>orf01781
(SEQ ID NO: 299)
VHAHTDKLCNGCNRIFNSIISHHTIFRERNKLSHKAIKSTRQEMGPCHVVFIEFFITLHR
RLIGNHDNFLTNLVGSGRVRNDGST
>orf01782
(SEQ ID NO: 300)
VNHCHWKLFIQNLGITESLIVTLIRMTDSHVVGTDKDMILLVNSLFLIFDIDKLRLS
>orf01784
(SEQ ID NO: 301)
VGNNDILWSKRTISINGFNDFLNTCIAVSTTLCNDDTFLIKRKIFIYKIFCMRNPVSMNT
NYNFFNTWLQDKFFNCMNQNRSIT
>orf01794
(SEQ ID NO: 302)
LVAPVASSTRFFKNNDSLTSWNNGFIIITINTIISYQRISKGQDLSIIRLVCNGFLVAGH
PCIKDDFACYINICSEGLAFKNCAIF
>orf01796
(SEQ ID NO: 303)
VVCYFYITIDWSWVHEDCCFFQTIVTFLSQAMLGMVVFF
>orf01797
(SEQ ID NO: 304)
MAFVLHTEKHHDINLINDFINGYKLSIVCKLLTSPFLRSREKEFSSQAFQNLHIGFGNA
>orf01798
(SEQ ID NO: 305)
VVQVTCNSNFKTLKVAKFLINGHQIKQALARVLARTISTIDDGSRNRWTSNQFSIVVDLW
MANHTDIHS
>orf01799
(SEQ ID NO: 306)
MCPCRILKEEIGNNRMVFIGNLGSIFKLNSSLDQFHYLINSEVFHGHHMVQCLLIF
>orf01824
(SEQ ID NO: 307)
VQFHLIIFQNLFCSLDIVIDSLTTNTKLLSYLSKTVIISVVKLYIIHLLICQKRRIKFKE
RIHTIGFFDSKLTIXXXFHSLIN
>orf01826
(SEQ ID NO: 308)
MHFHIIKLVNHFQLLIKLNRISHPNLHIKSSFLSLVLLFYQKEQDFAIMVI
>orf01840
(SEQ ID NO: 309)
MTGKKGFLFLNCHICMVTTTTCFLKERVESELLIFFYISPNRCLITV
>orf01843
(SEQ ID NO: 310)
LNRSILDNITLKHEVTSQKIEEVCKAVQIYDEIMAMPMKFNTIISEMGSNISGGQRQRIA
LARALINNPSIVILDEATSALDTINEERITKYIKSQGCTQIIVAHRLSTIKDADIIVVMK
GGKIVESGNHKYLMDLGGEYYSLYTKRK
>orf01845
(SEQ ID NO: 311)
LFKGGVTISRTPLSSEDTVMIDATEVKINRPKKKTISE
>orf01848
(SEQ ID NO: 312)
MAGKKDFLFLNCHICMVTTTTCFLKERVESELLIFFYILLNRCLITV
>orf01850
(SEQ ID NO: 313)
MYQDEAGFGRISKLGSCWSPIGVGPHVHSHYIREFHYCYGAVDAHTGESFFLIAGGCNTE
WMNSFLEELSQAYPDDYLLLVMDNAIWHKSSTLKIPTNIGFTFIPPYTPEMNPLNKCGKR
FVNVDLRIRPFELWKMS
>orf01852
(SEQ ID NO: 314)
LLQSPYAIDTINLKKDFLEKPIDIEKFKAFLEKEEIPLAIAWQGDSLHFYTKDRSILDNH
LDHLLEKMVNDPEKLSDFSMDKSLDDTIDEAKSQITFK
>orf01853
(SEQ ID NO: 315)
MKVVNLYDLKQMGNKGGCTIQLIHHFPFGMGLGHLKKDYIEFKRVGIFDGKAVEVTLREP
YSRDILQVVKSIKQRQKLIAYRYKEGKLLFVKKEV
>orf01876
(SEQ ID NO: 316)
MKPSGQEFPHPIFYSLFCFYTDTLGINSQVMQFSFERLLFQFCLNCWHLVMRSQNHNCRP
STRKVGCIGPIFFGHLLNHRKFSYQVLTIALMEEISLDCLPSGHHVSCQQGSNRYIGDRT
CSNSFLIRQFFRQDTTAVAST
>orf01878
(SEQ ID NO: 317)
LQVWYNLQSDFEQEITLIMWNPFANLVFNQPLISFFADLNLKILGYSYTDRVKTWPDIGT
GCRYNNLHLILLAP
>orf01909
(SEQ ID NO: 318)
LRQNRCYNCFHDHSCSWKSSRITSLHGCLVRFVGFDIHTHKRFIKSRNGFHDPTNNDGLP
ISHTTFKTT
>orf01910
(SEQ ID NO: 319)
LAAFTITSLKAKTKFHPFKGIDRDNSLSQSCIQFSIPLDIGTKTNWNASDDCLHNPTDGI
TITFDLVNIVLDFLFSFLVDNRNFRLGSSLLNFSDCQIFRNIDFLATKDHDMVGNLHIQL
SQETFGYCTNCHPHGGFTS
>orf01911
(SEQ ID NO: 320)
MTWARMSNFPLAFKAVFNVLRRHDVQPFLVVLIDDIHSNRRPCRLPVANARSKDNLVILN
LHTTTTTVATLTASKVLIDILSCQWKSSWNSLNNSC
>orf01913
(SEQ ID NO: 321)
MHDLAITGSRFDGMANSVAKIEVKTNTIVQLIFNHHLALHLTRMFNQGLCMFQNTLNRTI
QSRQESPQFWILNQAILDNFTHPFNQLSFSEGFKNKWINQNPIWLGKGPHHIFSKWCVNT
CLSTDRRINLSCQTSRNLNKVNTPHIGRGYKSSQVPNNATTKSNDSIATSQTLLD
>orf01915
(SEQ ID NO: 322)
MVICHNDYLLRLPEFSQPLTSLGHTTFFNLNIIRMMRNIDSDFHRRVSLSLLVFFC
>orf01920
(SEQ ID NO: 323)
LIEGHLVFADKPAQALVLLRKVGSPKKVSFLTLHLYFLILKIDILKITGF
>orf01951
(SEQ ID NO: 324)
MAVTKSQVFSRQGFDFSILGQDLTRLQDVSNLATIGTRIHKDSTANASWNTTSKLKAS
>orf01952
(SEQ ID NO: 325)
MTEGNASCFNQVSPSFCFNGIAINRNVIELVTQDDKSTNPTITNDDIACIAKNHPRDIFL
VGKFHNASQLKTISWKDQIISLSTYFCITIAMQGFLKTDINSF
>orf01965
(SEQ ID NO: 326)
MRLRDLRRVDFPDPDGPIKAVISLGWKDRETLFKAFFLL
>orf01968
(SEQ ID NO: 327)
LKNHSNVFTHFINVDFWTVDINSTIENLPSYFSNINGIIHAIETA
>orf01969
(SEQ ID NO: 328)
LHINPLNGFIFTIVNMDILSRKGYFFFRKGKDMLLIPVIC
>orf01981
(SEQ ID NO: 329)
MTIHIQVVKTNMVILADRLFQGFILRSTDKFFIKIRLVRSHNLRFNSMDFSTVAVHENKG
RHHMDELLPRFIINSKATVAKKSIVAQGFRFDGNFFRKTRQTNHLNIIFCDNPDQIIVFQ
NGLITNSQFNRLHP
>orf01988
(SEQ ID NO: 330)
LGHSKAEEHETICSHPFDDHTTETIPNQVKGRDMTSSETLPFPSKNQNQGKAKQNP
>orf01991
(SEQ ID NO: 331)
LLLSCRKVIVCFIFSSTWNKNFFNLAFSWNFDNCIRGFFSINSNLFGNMTSLWINIVGPC
RGYIAILSVNCNRIFTTVFCFIFFKTNSRT
>orf01992
(SEQ ID NO: 332)
MVKRRIRRGTREPEKVVVPEQSSIPSYPVSVTSNQGTDVAVEPAKAVAPTTGWKQENGMW
YFYNTDGSMATGWVQVNGSWYYLNSNGSMKVNQWFQVGGKWYYVNTSGELAVNTSIDGYR
VNDNGEWVR
>orf02006
(SEQ ID NO: 333)
MRFIVGRFTSFSLGIEFSPTSKLDDLLFKIAFLMILATWIKARKTKGAT
>orf02010
(SEQ ID NO: 334)
MANDNKSHYLIYRVLGISFEEGENIDLYQNKGRFLYKYAGSFLEEAAVLSFNEKFGTENT
>orf02019
(SEQ ID NO: 335)
LVNCKPLEAYRQLEEAELVGCWVHVRRKFFEATPKQADKSSLGAKGLAYCDQLFALERDW
ETLSADERLQKRQEELQPLMEDFFAWCRRQSVLSGSKLGRAIEYSLKYEETFKTILKDGH
LVLSNNLAERAIKSLVMGRSKRVQWTLLA
>orf02022
(SEQ ID NO: 336)
MFPVETEEITYKRKKSKGKCQALLAQFDSEEVHHQVEESICSDCQGDLKEIGATLQRQEL
VFIPAQLKRIDHIQHAYKCQACSDKNPSDKIVKAPIPKAPL
>orf02023
(SEQ ID NO: 337)
LKIIQQQSATIDSLTNELALLREQVAYLTQKLYGKSSEKSVCPSGQLNLFEEESPSEEDG
DVPS
>orf02024
(SEQ ID NO: 338)
LTIPVKDFKAVFTSTTKEKDLTGERIQFKVGFNQISQ
>orf02037
(SEQ ID NO: 339)
LVCQTIKYWHKFHLHIGRCKLLIGLIPVLNFFIRADIDCLLVLLSLIDRQNGKQFNLCQW
IIASNGLNDSFEIIESLIHRNILSDIICPNQKKNFIYCSTI
>orf02042
(SEQ ID NO: 340)
MIFSCSDSCFSIILLDGDIHENTTFSPLSILFISHRFNSLIGNEVPH
>orf02043
(SEQ ID NO: 341)
MPSKDNIRSPIDHLVIKSFLFFSWFQSILNTHLRHDNGDICFLLCPFNFSLHLIFV
>orf02045
(SEQ ID NO: 342)
MVSSKSPIAKSACLVHLLEPRSHILKIFMKVIGTVFFFS
>orf02047
(SEQ ID NO: 343)
VVEQIPVGHNSGSFFLFLLLRLLLSPLLRNSISFLTSQGIPWKLSNNKTKPIDKPTASKS
IATNPLLLHLR
>orf02054
(SEQ ID NO: 344)
MSCTKIGVPFKKLTKKACKGYKILTRLSSQWQKEAPNQSRSAAIXXHSIH
>orf02075
(SEQ ID NO: 345)
MNQIQIQIIQAQFFYRFFQSLTSLLIGLLTIPKLGSYEHFFTWNSAIFDCLTNTFFILIN
RRRINMTITSLQGF
>orf02080
(SEQ ID NO: 346)
MLLLISLTQLIIFLFFERFNLLLKTFLLVDLKSNKSA
>orf02095
(SEQ ID NO: 347)
LIFIEYKIADKTITIIGLSRVNVGRRIALLIAYFIAK
>orf02098
(SEQ ID NO: 348)
VIPRYVTKHQGWDHNPHTITNSDDDPATLVTFRTFKFNVGNCTIPKNDQNGSSQKFSGIL
QCPCEIHLLDSP
>orf02100
(SEQ ID NO: 349)
VVPKTATSTETKTITRIIHYVDKVTNQNVKEDVVQPVTLSRTKTENKVTGVVTYGEWTTG
NWDEVISGKIDKYKDPDIPTVESQEVTSDSSDKEITVRYDRLSTPDKPTPEKPEIPSPQE
PGTPGEPTPEKPIPQPNPEHPSVPTPNPELPNQETPTPDKPTPEPGTPKTETPVNPDPEV
PTYETGKREELPNTGTEANATLASAGIMTLLAGLGLGFFKKKEDEK
>orf02105
(SEQ ID NO: 350)
MAYSTDFKQRALDSIKEGHSHVEAAKFFGVGVRTLFTWEKKDVNKDT
>orf02143
(SEQ ID NO: 351)
MPILQAKIDSFRPLFDGLTSRFKVGAGLFLKRFSFSRVKQILLALPQFSMMNPIVNG
>orf02158
(SEQ ID NO: 352)
MAVQANWSFDITHDSSFFFSNQKRGLNFSQMCFKDRRRNGFFDRKIFKFKFNNPIQIF
>orf02160
(SEQ ID NO: 353)
LFLQIKGIKPNHTIALASYIEQSFFFINKTVHFKIGKQLIRTLQTNPFVIQLNCHLFQGC
KKKCSQALSLMVRLDD
>orf02168
(SEQ ID NO: 354)
MEIVLVSFSISFQHFIIAYCLDFSSAGFRNSQNFSNFC
>orf02180
(SEQ ID NO: 355)
LNFNHVYFGYDENRPVLKDITCSIFKGQKIAFVGPSGSGKSTIVRLLERFYKPLSGDILM
EQSSIYDFNLKEWRSKIAWVSQNNAVLSGSIRDNLCLGLNRLVTDDELMKVLDLVSLGDE
IRSMKEGLDTEVGERGRFLSGGQSQRLQIARTYLKDAEILIFDEATANLDADSEYAIISS
LYSVLKEKTVVIIAHSLSTVKDVDCIFFLEEGKITGSGTHKELLENHERYARFVQEQMIE
>orf02181
(SEQ ID NO: 356)
LIYAEKSFFDKSQSGELTSAIVNDMSVIREFLITTFPNIILSLVMVLGSIVVLFSLDWNL
SLLLFITLPCMMFIILPLSNISEKYSRRLQKEIGFLTGQLTEKIQEHELIKTNQAEKSVQ
NVLDNCIERVQNNSLKSDRVTSFETPFALLFIFATIAVMLTYGSYRVSAGYISVGTLVSF
LIYLFQLLNPISNIANFVTVYSRSKGS
>orf02183
(SEQ ID NO: 357)
MKLKLLRVDTKVIMGSFFLVLSSLLALLLPLILKGLIDGSSIENIGSKVFQSFLIFIGQA
LFSSIGYYLFSQSGEKKIAKIRKKVI
>orf02196
(SEQ ID NO: 358)
LIRYLDQYEDVILREIKAQFPDVAVDKLMEEYIKAGLILRENKRYYLNFPTLESLDSLEL
DQEIFVREASPVYQALLEQSFETELRNQINAAILVEKTDFARIKMTLSNYFYKVKQQYPL
TEKQQELYDILGDVNPEYALKYMTAFLLKFLKKDHLMQKCRDIFVDSLVVLGYIVQNEDR
KYELAIDFDKERLTFYLA
>orf02197
(SEQ ID NO: 359)
MIGLKEVCRFLTDNTSLSTSMINHPIQINGNMAIVTCGSLDGLSHV
>orf02206
(SEQ ID NO: 360)
MLINDLTFFIFDISPIQSYKKVRLEITNLWNNTKNATSRGDSC
>orf02208
(SEQ ID NO: 361)
MRERVRLSGSLFTSLKTREHIKSTMELFHKYVFFLIQEIKIKMINFLKIGDLPTL
>orf02219
(SEQ ID NO: 362)
MIDHFEIKVKDLQISEGFYRSFLAPLDYKLTFKTSSLISFLSPNSPHPGGDFWLTQGTQD
PVHFAFLAENKEEVQACYEAGLEAGGRDNGVPGYRSEHPIYYAAFMIDLDGNNIEVVCHK
E
>orf02221
(SEQ ID NO: 363)
VIVFLSRNKDGNAFCHLDLISIANPVWGWDDDFITWIDHSHKEGIERIFGSRSDCHLI
>orf02224
(SEQ ID NO: 364)
LSNQFYFSLQTKPILKVKQFLLFQSQMTRVSEILQFSNKL
>orf02227
(SEQ ID NO: 365)
LFRLGQLISLNVAVHKPIKKFQGWIVLSSLPFQSLDILKFFRRFLSRYLVETLQLTGRIE
SQGIKHGLTFWF
>orf02229
(SEQ ID NO: 366)
MEDKEMGFYLMVASMLLGLLALKIGFSQFKEKKDKFLSILTSLAGTALVLVAVWLGWPK
>orf02250
(SEQ ID NO: 367)
MLDSDIGCSRKNLLGLFWIRRRRNIHIVDRAMEKGISNRAPNKISLKACFFNFF
>orf02278
(SEQ ID NO: 368)
LLHPFTRNITCDRHILTLLGNLVNFIHIDNATLCTFDVKVSNLQEFEEDIFHVLTHITSL
RQSCRIRNSKRYIQALSQGLGKESFP
>orf02279
(SEQ ID NO: 369)
VEIDAFVVVINRHCQGTLGTILTNYIVVQDMEEFNWFWHLRQVCQDFLNQFFSNDFLSQL
HAFITNKSIVASNYFLYFFLVFATK
>orf02285
(SEQ ID NO: 370)
MNXXGKGEEGEDLVVGVFKWLYSERLRFDTERVGGGGKGK
>orf02291
(SEQ ID NO: 371)
MHKNFVVVVTNFFTAVQFIQFNKEGTTCHNTTKFFNHLDSCLNSSTCRQKVIYNKNTLTW
LNGIRVHSQGIDTVLFFIVSRNNFAWQFTWLTNRRKTNSQLKGNWTTHDKSTSFRSHDHV
DFLVSSILNDFTNSVAISISISHQRTNITEGNAFLWIIFNCCNVIF
>orf02299
(SEQ ID NO: 372)
MLNRQVCFCFVNHISPLNVVIWENLSLEELLYAICICFITHKIAKQTSLTIDNAGIAMNN
IR
>orf02304
(SEQ ID NO: 373)
LDSRFFCTDFFKGRQAKGCSFSCTSLSLTDNILAFKGQRNSLFLDRTSFYKTSFFNFC
>orf02315
(SEQ ID NO: 374)
LLRKQEREYLRAENAILKKLRELRLKEEKEKEERQKLFKN
>orf02320
(SEQ ID NO: 375)
MRFLADQDRIQHHRYSWALFDKVQGLLSHTDSREKTNLNSPKFHITQAI
>orf02321
(SEQ ID NO: 376)
MLKNGIISWKDFKSFFCQGCQTSHCYKPMQVVQGIGSQISRQSTTTKNIISRKC
>orf02324
(SEQ ID NO: 377)
VTAHRIFGTSSIHSKLIGLAMLGITAMKIICHKLNRNHINIFRRLGIQGKTEFLLIHLIR
QVKMNDLSQGMNPTICPTSTVNSNDLPFI
>orf02328
(SEQ ID NO: 378)
MLARSKNCFMKSLSIFLLIFYFFDSYQISKKRRSLIGL
>orf02339
(SEQ ID NO: 379)
LEVCIHHHHQISCRILQACIKGCFFAKISRERNIMDCRILLPIGL
>orf02345
(SEQ ID NO: 380)
LTGNVICHPKLPDKISVKCLYSSKIQFKPRQRRLAMGMVTDFVSSIHNLKAAL
>orf02356
(SEQ ID NO: 381)
MGRKPKKRPEERTELERLQAENEYLRAENAILKKLRELRLKEEKEKEERQKLFKN
>orf02360
(SEQ ID NO: 382)
MDEIKNFRQWGSKTPGHPEVTHTSGVDATSGPLGQGISTAVGFAQAERFLAAKYNKDGFP
IFDHYTYVIAGDGDFMEGVSAEAASYAGHQALDKLIVLYDSNDICLDGETKDTFSENVRA
RYDAYGWHTVLVEDGTDLAAISTAIETAKFSGKPSLIEVKTVIGYGSPNKSGTNAVHGAP
LGAEETGATRKFLGWDYDPFEVPEEVYSDFKTNVADRGQEAYDAWASLVSDYKVAYPEVA
SEIDAIVAGKSPVTITEKDFPVYENGFSQATRNSSQDAINTAAVLPTFLGGSADLAHSNM
TYIKADGLQDKYNPLNRNIQFGVREFVMGTILNGMALHGGLRVYGGTFFVFSDYVKAAIR
LSAIQELPVTYVFTHDSIAVGEDGPTHEPVEHLAGLRSMPNLTVIRPADARETQATWHHA
LTSTTTPTVIVLTRQNLVVEEGTDFGKVAKGAYVVYDTPGFDTIIIATGSEVNLAIKAAK
ELVLQGGKVRVVSMPSTELFDAQDATYKEDILPSKTRRRVAIEMAATQSWYKYVGLDGAV
IGIDIFGASAPAQTVIDNYGFTVENIVAQVKSL
>orf02362
(SEQ ID NO: 383)
LNFLNEPRRQGNIGNKMAIHNIDMIRCYFIIQKSNLLFEFVQIHGHQRW
>orf02367
(SEQ ID NO: 384)
VIYSYDYPRLLHSRTLAMGNSNIIPNTGLSFCLSLIKTFDKLVSIRHITRLNQ
>orf02371
(SEQ ID NO: 385)
VSRKQEQMETLLLLLRDSKDYISAKVLGEKLNCSDKTVYRLVKGINKDCPVEAFILSEKG
RGFKLNPRSSLVDVDGNFTEAFDPEVRREKLLERLLLTAPKPHSIYDLGEEFYVSESVVL
KDKRQLQESLAIYGLDLKMRQRKLFIDGDEAQIRSAILNLLPMFNQLDLEQITQNKVQPL
DGELAHFCLGLLITLERELGVNIPYPYNINIFSHLYIFISRNRRSTSIHVVAPSKPTIVD
EKIYSVCQKIIQEIEQYFRMKVDAVEIDYLYQYVVSSRLQKPFSSGKLPFSQRVLDVTHY
YFSRMCMDNREIETTDPDFVDLASHISPLLRRLDNRVQIKNSLLSQILLTYPNLVKELTT
ISKEVSLVFGFASLSLDEIGFLVLYFARFQEKRARPLKTVVMCTSGVGTSELLRARLEKQ
FSELDIIDVVAYHQLDELINLYPDLDFIVTTVALQEPASVPFVLVSAFLTEGDKQRLQAK
IQEINYE
>orf02390
(SEQ ID NO: 386)
MMSMVDPIDQTFIVNLKIRKSQVFSQLQFSCHIVVYPSEVHIYQALVIKLQNHILGPQVL
P
>orf02391
(SEQ ID NO: 387)
LANRTRIDNQLPTSPVTKQLLVNMSINSNITGRMSHQAVKLLLFASMNQLSPPVLIRQMM
ANSHRQIPKLTMNLKRLIVEHFNFF
>orf02395
(SEQ ID NO: 388)
MDSIEFFHDKTFLFYLTSFYSKRMGVTTKMIKIAEGRFS
>orf02406
(SEQ ID NO: 389)
LTRFEEIFEEYKNPQDTFFYPLVYKENTYKKTAISIFALLMLGVCCLFLFSQQSYKKLVQ
YYANDQNLPSRITYSEYSDK
>orf02414
(SEQ ID NO: 390)
MIAEFIDGLQKFHFLQNALITAIVVGIVAGAVGCFIILRGMSLMGDAISHAVLPGVALSF
ILGLDFFIGAIVFGLLAAIIITYIKGNSIIKSDTAIGITFSSFLALGIILIGVAKSSTDL
FHILFGNILAVQDTDMFITMGVGAAILLLIWIFFKQLLITSFDELLAKAMGMPVNFYHYL
LMVLLTLVSVTAMQSVGTILIVAMLITPAATAYLYANSLKSMIFLSSTFGATASVLGLFI
GYSFNVAAGSSIVLTAASFFLISFFIAPKQRYLKLKNKHLLK
>orf02424
(SEQ ID NO: 391)
LNQEIIWKTRKSFTFKSRSLTDIRSRRKCLTNIQLSLIVRHLRQSRTLLAELNVNIPKRQ
IGPILTRFWPN
>orf02437
(SEQ ID NO: 392)
MQGEMRFSLVQFLTTLIKFCIFPFLLPNWLFGRKACTHWEFSFPKIKGVFEFHGISFINN
NKLKTTTSKKDENRGTTFIRKKI
>orf02438
(SEQ ID NO: 393)
MYEEPEVAPVHPTGPTPATETVDSIPGFEAPQESVTIL
>orf02468
(SEQ ID NO: 394)
MRLSWHFMRFKKLPLLINQTILDVGSTDINCNVICHKYLLLD
>orf02470
(SEQ ID NO: 395)
MGNNGQFTFGYRHDFFQNQLAIFNALVDTFTRRTIDIKTLNTFINEVLNQGTRTFWAYFS
LIIITCVEGWNDTFVFFQI
>orf02497
(SEQ ID NO: 396)
LSTRNKYCKNLIIFESTFNILDIVKKDLKLNSKLEKDLKY
>orf02499
(SEQ ID NO: 397)
MNRSVQERKCRYSIRKLSVGAVSMIVGAVVFGTSPVLAQEGASEQPLANETQLSGESSTL
TDTEKSQPSSETELSGNKQEQERKDKQEEKIPRDYYARDLENVETVIEKEDVETNASNGQ
RVDLSSELDKLKKLENATVHMEFKPDAKAPAFYNLFSVSSATKKDEYFTMAVYNNTATLE
GRGSDGKQFYNNYNDAPLKVKPGQWNSVTFTVEKPTAELPKGRVRLYVNGVLSRTSLRSG
NFIKDMPDVTHVQIGATKRANNTVWGSNLQIRNLTVYNRALTPEEVQKRSQLFKRSDLEK
KLPEGAALTEKTDIFESGRNGKPNKDGIKSYRIPALLKTDKGTLIAGADERRLHSSDWGD
IGMVIRRSEDNGKTWGDRVTITNLRDNPKASDPSIGSPVNIDMVLVQDPETKRIFSIYDM
FPEGKGIFGMSSQKEEAYKKIDGKTYQILYREGEKGAYTIRENGTVYTPDGKATDYRVVV
DPVKPAYSDKGDLYKGNQLLGNIYFTTNKTSPFRIAKDSYLWMSYSDDDGKTWSAPQDIT
PMVKADWMKFLGVGPGTGIVLRNGPHKGRILIPVYTTNNVSHLNGSQSSRIIYSDDHGKT
WHAGEAVNDNRQVDGQKIHSSTMNNRRAQNTESTVVQLNNGDVKLFMRGLTGDLQVATSK
DGGVTWEKDIKRYPQVKDVYVQMSAIHTMHEGKEYIILSNAGGPKRENGMVHLARVEENG
ELTWLKHNPIQKGEFAYNSLQELGNGEYGILYEHTEKGQNAYTLSFRKFNWDFLSKDLIS
PTEAKVKRTREMGKGVIGLEFDSEVLVNKAPTLQLANGKTARFMTQYDTKTLLFTVDSED
MGQKVTGLAEGAIESMHNLPVSVAGTKLSNGMNGSEAAVHEVPEYTGPLGTSGEEPAPTV
EKPEYTGPLGTSGEEPAPTVEKPEYTGPLGTAGEEAAPTVEKPEFTGGVNGTEPAVHEIA
EYKGSDSLVTLTTKEDYTYKAPLAQQALPETGNKESDLLASLGLTAFFLGLFTLGKKREQ
>orf02501
(SEQ ID NO: 398)
VDRTDEVSSKHCFEVVDRTDEVSSKHRFEVADRTDEVSNIYTARQS
>orf02512
(SEQ ID NO: 399)
MSCNCAFYRSQFFDVNSVSNYHSHQKELRFPNSILFTYFVKVT
>orf02535
(SEQ ID NO: 400)
VGLIKLTSYVFVCISNSFLTRHDKNDNICFFHGNFCLVLDLFHERSIDIINSSCINHAKR
TIEPLTRCINTVTCHSFDIFYNGDSLTSDPIK
>orf02537
(SEQ ID NO: 401)
LSSKSCIDRTNQETFHTLGLEGVGMKSGSLFCSVQISDKEKENSRLANGFLRYQFIQGIF
LLLTSYHNHRVGLEILPR
>orf02554
(SEQ ID NO: 402)
MKSKEQTRKLAVGCSKYSFEVADKTDEVSSKHCFEVVDRTDEVSNYIYGKAKLTWFEEIF
EEY
>orf02556
(SEQ ID NO: 403)
LSNSFFLIKFSSSKISGKKRIVSDNIFIRNKFICHFKKE
>orf02564
(SEQ ID NO: 404)
MDYSKVAAEVIEAVGKDNLVAAAHCATRLRLVLKDEAKVNQAALDNNADVKGTFSTNGQY
QIIIGPGDVNFVYAEIIKKTGLKEVSTDDLKEIANKDKKFNPLMDLIKLLSDIFVPIIPA
LVAGGLLMALRNFLTSPDLFGPQSIEDMYPAIKGFSAMIQLMSAAPFMFLPVLVGISAAK
RFGANQFLGAAIGMIMTTPDLGGKEAFWDILGFHVTQTNYAYQVIPVLVAVWLLANLEKF
FHKKLPSAVDFTFTPLLSVMITGFLTFTVIGPVMLVVSDAITNAIVWLYNTTGAFGMGLF
GGTYSLIVMTGLHQSFPAIETQLLSAYNNNGTGFGDYIFVVASMANVAQGAATLAVYFLT
KNAKTKGLSSSAAVSAFLGITEPALFGVNLKYKFPFFCALAGSAIGAFVAGLTHVIAVSL
GAAGFIGFLSIKAGSIPMYIIAEIMSFVAAFAFTYFYGKTKAASVFADEAATATAETVTE
PTVEAPVVEETDTLQNETLVTPIVGDVVALADVNDPVFSSGAMGQGIAVKPSQGVVYAPA
DAEVSIAFPTGHAFGLKTRNGAEVLIHVGIDTVSMNGDGFEAKVAQGNKVKAGDVLGTFD
SNKIAAAGLDDTTMVIVTNTADYASVAPVATGSVAKGNAVIEVKI
>orf02570
(SEQ ID NO: 405)
MTESYTWVEADRATLSRYRHGQGHLTDQFFSFKVQRPAAKTLIASISTGKGMGPSFDGTP
VITSGNQNRINTIKNSFIMSSSSVRISLRKLTSQRNFLRNLSSLILLAAQVAKGDATACS
HQRIGRVVGQDSHETLSLTEFF
>orf02571
(SEQ ID NO: 406)
MNINNEKVWFAFYLLDMQITRPTSTFNDRRIGLIGKLQELRFLAGNLLLR
>orf02572
(SEQ ID NO: 407)
LIKGYLPNHLSLMDLCSKTTCTLDDFAGIAGRRNHRGFFCHIGNGVFLTVDKYLWNQ
>orf02578
(SEQ ID NO: 408)
LIKLTGRNFSDILIKCLVKCFTNLLSNQLMLLPSTLKL
>orf02582
(SEQ ID NO: 409)
MIQSENHCSASHSNRDYQSQHDNQGRTCQCFIIVPCHKKGSCSVGEITWNQRCQNGQDKD
HSRCLIKNT
>orf02589
(SEQ ID NO: 410)
MIARQLMVFFSTNQADTRITNMSIDSLIINNSKDFQSSSHASVSFILTKLVNLLIFNF
>orf02590
(SEQ ID NO: 411)
MGEPFTHFIDCIDLGINPSYTQVCHRHFTSDIPCAMTSHPIS
>orf02597
(SEQ ID NO: 412)
LSSDSHFIGIKAFVILILGKSNSIVLRIVGLYQDLTCLFSTTCSTCHLSQELEGSLRRTE
IRQIQGRIRI
>orf02598
(SEQ ID NO: 413)
MAVHSLGIHMQGQRNIAVGTSIHRPTLPTHDKARITTAIEHENHLLFFNQTVLDSL
>orf02599
(SEQ ID NO: 414)
MVTGIAVLLISRFMLFINNHDTQIFQRSKDSRSGTNNNLGIATLHLAPFIILFTIG
>orf02600
(SEQ ID NO: 415)
VKNGYLVPKTCYKTLGHLRSQGNLRYQQNSCLALIQGTLDNLQVNLGLPTSCNPLK
>orf02601
(SEQ ID NO: 416)
MVNLIPRLGLDLLLIDCLIFQTKQAFSSQTHHFSLLGKV
>orf02602
(SEQ ID NO: 417)
LGLQTKNNPLNQAITLTKRHMNPHPNFQHSLKFLRNPVTIGLVRLHQGHIYDNLS
>orf02605
(SEQ ID NO: 418)
LGNHFCTICSTTYQAFLQFIQIWWCQEDKDSIWNLFLDLKSTLNFNFKENIDSLVQGFID
IGQRSSIVVADIFCVFQHLSLTNQLFKFFTSTEEIVNTVHFSRTLCACRHRYRILKLVFR
TLKNLSSNRSFSNP
>orf02618
(SEQ ID NO: 419)
VLLPNVLIKFINRFCWNVIPIKGCDSTFWNNKLIAIFKGNLDRGIHTIFCLHTT
>orf02628
(SEQ ID NO: 420)
VGCSYICHELVTNHDHFLFVIVEFLHSTVNTKCEGLQGPVNVINPKFLNCSLNAFFGVI
>orf02629
(SEQ ID NO: 421)
LLHLWRSIRVVPSNEGIIQIDQNSLDSLRLQAGDCQIIDCFHSKIWYIIFNRHSGSFS
>orf02631
(SEQ ID NO: 422)
LFGSCRQINHTSLQIRQGKEVFLGSSLAQEVIDLIPTLVHLLNDRIVGIADDFQTGKEKL
INRKRVMALQITSHLFNDIGVLGITNGNQATMLDNKGHGKSLIVGXXHSIH
>orf02633
(SEQ ID NO: 423)
VKGLLLATKLCRTNSHTDNLTRYSNRSICQNDLISHIQLTFKEDEKAIDDIRQKALGSHT
NRYPSNTSSSQQTRNWQT
>orf02635
(SEQ ID NO: 424)
LWGILGLTLPNLSGIGLLGDLFVGGLKAVAPILVFALVANALSQHQKGQDSNMKTVVFLY
IL
>orf02637
(SEQ ID NO: 425)
MIGTFAAALVAVLASFIVPIEITLNSANTEIAPPDGIGQVLSNLLLKLVDNPVNALLTAN
YIRILSWAVIFGIAMREASKNSKELLKTIADVTSKIVEWIINLAPFGILGLVFKTISDKG
VGSLANYGILLVLLVTTMLFVAPVVNPLIAFFFMRRNPYPLVWNCLRVSGVTAFFTRSST
TNIPVNMKLCHDLGLNPDTYSVSIPLGSTINMAGVAITINLLTLVTVNTLGIPVDFATAF
VLSVVAAISACGASGGASGIAGGSLLLIPVAGSLFGISNDIAIQIVGVGFVIGVIQDSCE
TALNSSTDVLFTAVAEYAATRKKASLLMSCLLRFYSNLLGNSYVY
>orf02639
(SEQ ID NO: 426)
MIAHAIIILLFKNAGKLCFLVVFFFTVHTFGKNQLLLG
>orf02645
(SEQ ID NO: 427)
VFEVVDKTDEVSSKHCFEVADRTDEVSNHTYGKVKLTWFEEIFEEYHTKKPCSSR
>orf02651
(SEQ ID NO: 428)
MKNRIIDVFEVVNRILVITVENPDFEDLRVNQFVKIGDKKYRVRSGPMIHSTPPQSVLDR
DTFTIDYTDDELLDKEAVFTTH
>orf02657
(SEQ ID NO: 429)
MAYIEYKQRGKKRLWSFSIRERSKSLLHKSGFKTKREAKIEAEKVLHKLNTGSVLSSSMT
LSELYNEWLDLKILPSNRSVVTKKKYLMRKKVIERLFGNKPVSQIKPSEYQKIMNEYGET
VSRNFLGRLNSSIQASIQMAIADKVIIEDFTAYVELFSSKSGQKVEEKYLHTESDYQKVL
VYLKNKFDYQKSIVPYVIYFLFKTGMRFSELIALTWDEVDELNEQLKTYRRYNTAIHKFT
PPKNNTSIRLVPITSDMLSLLKTLKILQLKTNKELNIDNENNLIFQHFGYVYDVPDIATV
NKAIKVMLKELQIFPLITTKGARHTYGSYLWHNNIDLGVIAKILGHKDISMLIDVYGHTL
EEKISEEFTAVKSLL
>orf02658
(SEQ ID NO: 430)
MKDTISNKDLISMGYRPSTANAIIHQVRELLVSRGYTFYNRKRLMVVPKSVVKELLGMEL
>orf02659
(SEQ ID NO: 431)
MNKEVLNRAPSNSPITIHQMSNKSYSKFQEEVSLKYGFIGLKLDKLSLTAEVSEEFHSEI
LSGNFTLYDYFGVVEPNLNRNGELASYKGQFFNDEKENWYIEYTPVASIKMNKRPLKIEF
TPSKVSKKNFLIVFHRMFPYMFNIAISTFHLAYDFERDLSALRVNWPKVMYRPIYKGMKL
ETMDFGAPKGNYHLTAYNKLKERMDSGDMAEIEIYQQYDNLWRIEYKFYNEGNIKKELKN
GLPFLAKIPVYIENFKGLEFNNLGVNEKIYLFALRNKPELFAESDKRTVAKYKKLAESIS
EVNLNVFFQNALDFVEIFNQEPCLINFFEFMNSMLQGDIPKLTINQE
>orf02660
(SEQ ID NO: 432)
MHFDKSKFGAVFSAPGLYEVEVINNASFGQNAQYEVIQSRKLGTFAELIEMAKIK
>orf02661
(SEQ ID NO: 433)
MFKIKQTASLSEHYFLNTSKLRSIRWFTIGFLSILSLYSCILFKGWFLQMFTLSVGLIVT
LYFERKIKGCFHQIEPLLIVRENLLFMLRRNSFLFTATKDGAILRSAKFNYQLNDVSIVI
QALKSGDEFTREMDDLDVLLSSVLGISLSYKEIYATHVEYVFVYRQPERLHITSLPLEED
NSLKIKIYDDFIIDLRKNFSMLISGASGAGKSFFTYYYLTRFISQTVNGRHAKIYVIDPK
LSDIYKLSKFSGLPVENYGTTNEDAFRIVRHYINEMNRRMEIYNKSDLFDSIGIDLGLPP
LLLVIEEYSSLVASMDSKAKKDFENMVAIVAQKARSLSMGVCIVMQQPRSDSLSTNIREQ
LVNAIFLGAPTRESSQMMFGTTDVPKVKKDKGVGLYSTDREPPKEFHSPMFDRDVFEVIL
PVWEWAAKDYMKDEDEDV
>orf02662
(SEQ ID NO: 434)
MKQKQPIVSRTKQHTFEELIQDQKLERLANLSPDLVGRYGFTASCASSFANLIKEAYGGK
NLNVVYASRMLALWNIACSCYHKADGYSLADALFSDKKICLDYFYYHNNTSDIITLDMIE
DVKKNYLQLVTTATSDNMSVIEFEMEKESDLYYFIKATLGSSFSRMHYSVLVKALAGALA
KNI
>orf02664
(SEQ ID NO: 435)
MGWKGTPPCLHPSNQDTTILIVQQCLRRIEVLAMINFLN
>orf02665
(SEQ ID NO: 436)
VFGSYYGVIASIFFKEFWITEISSNQLIWQVCSSYNWILGNLFKVNPVI
>orf02666
(SEQ ID NO: 437)
VLPSHQVLTFSMSPVHRPPNTIIWIELIKEMVFSTKIDKSIWIIDPTNLS
>orf02689
(SEQ ID NO: 438)
LIVSLKTKSRKAKDMAESIQGWLAQFLVNLFKSITFDCGKEFSKWKDISNHHDSESFFAN
LGCPRQRCLNEHSNRLLRCHDLPKQTDFNEVSQEF
>orf02690
(SEQ ID NO: 439)
VVEIIYFLIIIIASGLGSISGMGGGIIIKPLMDSFGYHSVSDIAFYSSFSVFIMAIISTT
KRFSQSKEIKWRLIFTVSFSSVLGGFLGHLIFQVLLSQLSVRLVSIVQMILLFVMLLVSF
VLTDFKKTYQFDKIGFYMICGLLLGLISSFLGIGGGPLNVSLLMVFFSISIKEATMYSLA
IIFFSQLSHLATIVVVTGLNQYHLAPVPVIFLASICGGVLGTVVSKVLPENWVRYCFKGM
LFFVMGMTLYNLFHIL
>orf02691
(SEQ ID NO: 440)
MMGTNSEEGFLDDFEGPQVAVSVKDFSIADTPVTNQEFAQFVKETGYKTLAERQEWSFVF
ILFVPEAEREGYPHPAGAPWWLQVSNACWKHPYGENSNLVGLEDHPVVHVALEDALAFCN
WSGMSLPTEAQWEYAARGGRQSEYPWGDTLLEGGYYHANTWQGRFPYENTALDGFIGTAP
VYEFLPNDFGLYQMIGNVWEWCRNPRYTLLASFNEDDYELPKYGIQDEEYAIRGGSFLCH
CSYCNRYRVAARNGCISTSTSSHLGFRCLKE
>orf02694
(SEQ ID NO: 441)
MVQTKQPNIILIVVDQMRADALSLNSKDKLVSTPTLDMMASVGYNFENAYSPVPSCVPAR
AALLTGLDQDKSGRVGYQDEVPWNFTNTLPKVFKDMGYQTECIGKMHVFPSRQRLGFDHV
LLHDGYLHVDRKYDKTYGSQFDYASDYLAFLKGKVGYDVDLIDDGMDCNSWEARPWDKDE
KLHPTNWVVSESISFLQRRDPTVPFFLKMSFEKPHAPLNPPKYYFDMYMERLPQFLDLHI
GNWEVLEKQIPSIYALRGKLKEDDQRRMVAAYFGLITHIDHQISRFLTALKEFRHDKDTI
IWFVSDHGDQLGEHYLFRKGYPYQGSIHIPSFIYDPAGLIAGNRGTIKQLVKIQDIFPSL
VDLAGGTTTDELDGRSVKNLLFGQYEGWRTEFHGEHALGKDSSQYILTDQWKFIWFPVLN
HYQLFDMKKDPHEMNDLYPSEKYQPIVRQMKKKLVDFLRYREEGFVVDEELVPVELSKIT
PTLTKTGDSQS
>orf02696
(SEQ ID NO: 442)
MNTMLDKMQEKLSPIAMKVGNQKFLVALRDSFVGTMPVIMTGSIALLLNAFLVDLPQQFH
LESITKTFQWLVDINNLVFKGSIPIVSLLFIYCLGVNIAKIYKVDTVSAGLVSLASFVIS
IGSTVTKSFPLANVGDVKLDQILQGIDNLAFDGKNLMVTIGNVIPGNHINARGYFTAMMI
GFLASIIFCKVMKKNWVIKLPDSVPPAIAKPFTSIIPGFMAMYIVAILTYVFHLLSNDLL
IDWVYKVLQTPLLGLSQSFFAVILMIFLNKLFWFFGLHGGNVLAPIMEGLFGVAMLANLD
AFQKGEPIPYIWTSGSFGAFVWFGGLGLVLAILIFSRNSHYRKVAKLGLAPVLFNIGEPV
NYGLPVVLNPLLFIPFVLSPVFMATVAYWATSWGLVSPVTQNVTWVMPPILYGFFSTAFD
WRAIILSVVCLIISVLTYFPFVKMADKTELS
>orf02697
(SEQ ID NO: 443)
MDESNLESVMGLIMYGGEAKSNAMEAIQAAKKGDFSKANRRLADANAALLQAHKAQTEML
TREAQGEETSISLLMVHAQDHLMTSLTFVDLAKEVVEVYERFEKN
>orf02698
(SEQ ID NO: 444)
MAKVTIMLACAAGMSTSLLVTKMQKAAEDKGLDAEIFAVPAPEAEEIVATKEVNVLLLGP
QVRYLLGDFQEKLKDRQIPVAVIPMTDYGMMNGSKVLDLAESLLD
>orf02699
(SEQ ID NO: 445)
MKRLISANPSEILQMNAEELKQSILASEGRVVLSENVVTRETFVGDITNSEIARAFGADM
ILLNCVDVFEPKIYALDSSGDDVIHRLHQLVACPIGVNLEPIDPSAKMLEETQEIVAGRV
ASVETLKRIEELGFDFVCLTGNPGTGVSNREIIKAVQTAKENFSGLIIAGKMHGAGVNEP
VAELSVAEQLLEAGADVILVPAVGTVPAFHDQELREVVDLVHSKGGLVLSAIGTSQETSD
TDTIKEIALRNKICGVDIQHIGDAGYGGLATVDNIYALSKAIRGVRHTVSRLARSVNR
>orf02700
(SEQ ID NO: 446)
MEKLLQEKLLPVAARLGNNKALVSIRDGITLTIPLLLIGSLLMVIASFPIPGWEKYLGDI
GVADYLWKGVDSSFGLLGLVASFGIAYFMARQYKVDGIPAGIVSLSSFITVTPFITGEAG
AGMPTAFMASKGLFVAMILGLINGYIYQWFINHNIQIKMPDGVPPAVSKSFSAIIPGAVT
IVGWLIVYATLDKLSLPNLHEIAQVALGGPLGLLGNNVIGLLILIFLNSSFWFVGLHGGN
VVNAVMKPLWLANLDANKVAYQTGETLPNIFTSVFMDNFVFIGGGGATIGLVLALGYLAH
KKKASKQLKTLAPITVIPGLFNINEPAMFGVPIVLNILLLVPFILAPMFNLLVAWGAMAS
GLVPLTYTDPGWTMPPVISGLLATGSISGSLLQIVLIVLDVLLYLPFVIAIEKRFKLLED
>orf02701
(SEQ ID NO: 447)
MTLSKKQLQLRAKILETVYTLGPISRIEIATKTGITPATTSSITNDLIKENILLELGEDE
HDTSVGRKKILLDIQAKRFYYIGCELSEKHFTFALGDNLGNILKEEKEIVTKQLIQEKGN
QLINQTLKQFLNNCSDYEIEAIGIALPGRYLDDYKITTNNPLWQHIDLEMIQSHFDKPLF
FSNNVNCMAIGKRLFSRQQNDTNFAYFHFARGMHCSYIYDGNIYGKGNLMIGEIGHTVVS
SEGEECSCGRKGCLQTFAGEAWLIKKSKILYHQSPYSLLPSLVKNADDIDIQVILTAYQL
GDTGIITLIHQALLYLSQTILNISMMIDSQKIYLHSPLLTNQHIIQKLYSEMNYKPKLLY
NRLPEVIIEPYNDFTAAHSAIALCLYHTILHS
>orf02719
(SEQ ID NO: 448)
MPFKENLICQHRNHHCSVFFISLGLLHNIHIEIDISQTRASFLDLSDYLQAVLMILQKFC
QAIGLAQRLDLLQLHLLHLTRLLL
>orf02728
(SEQ ID NO: 449)
MYLLLLVVKDHIALIDKEMHVWRPNCILRDLTNFFIKRNHIVTNKTNGSPTKREV
>orf02729
(SEQ ID NO: 450)
MVLALMNHFIKEIQGIPINRLTILIENSIFKLNLKNWIIG
>orf02731
(SEQ ID NO: 451)
MKIKEQTRKLAAGCSKHCFEVVDRTDEVSSKHCFEVADRTDEVSSKHCFEVADRTDEVSN
HTYGKATLTRFEEIFEEYKGVPR
>orf02743
(SEQ ID NO: 452)
LVEQLTFNQWVTGSSPVRVIYAGLAELADAPDLGSGA
>orf02745
(SEQ ID NO: 453)
VCQRMDARTCKTTIIAVHNVLTALQQTWIAVQLYQTK
>orf02746
(SEQ ID NO: 454)
LHLGKSILSLPVKGKDLEFLVHLFVINHWIGFPSRTSTFCRCKVLNGME
>orf02747
(SEQ ID NO: 455)
LEQTVIIANNPCELYWDNHLSFLSDSLLKQVIVHLKRICLDIHHDRGCSHVRNDTT
>orf02749
(SEQ ID NO: 456)
LTDDGVLILVVDAGWRGNSCLQEQGCHHFRAILLCITWHFRSCTDKGHLTFKDIDQLRQF
VQTDTSDEISNLGNTAIVSRSHQTSFFIRIRHHGTKLPNLEPTVVLGHTLLLVNHWPLAI
QLDPNAQDEKDGRGQNQ
>orf02750
(SEQ ID NO: 457)
MLKMRKMGEVRTSKIKAKTQSKQRLKISEPFLLETSW
>orf02765
(SEQ ID NO: 458)
MDYNAVIPEFLVSNIEQSRSFYCGLLGFRIEYQRPEENFLFLLKSVN
>orf02766
(SEQ ID NO: 459)
MLEEGTKDQLAELTYPFGRGVNLSFGIKDVSKLYQKVMEANYPIYRPLTKRKFRVSDPYI
YPHKFAVLDPDGYFLRFSE
>orf02771
(SEQ ID NO: 460)
LACDKHGKGCFTDISALLIGDIHIHHTGCTTLMDTFSFNGQYIVEFSCLKVVDRGLECHP
IKSQRDNHQTTDLVT
>orf02773
(SEQ ID NO: 461)
MGIAIVVERRVHYFGRHHNVTISHFFNFVIFKGRYSVKMKVFHRFLLIFQTTL
>orf02784
(SEQ ID NO: 462)
MIACRHDICKSQKGLEHPFCIVRRLTRDFNQRPVCIVEANIFCLKITPQIIANMIVARTV
KSSKTGITLTTSMCKRDNHKITWFHRRNGFPSFFNNPNRFVSTIFVSNFRFWITVPP
>orf02799
(SEQ ID NO: 463)
MNMNKDQIAILNGADNLNLTLWMTLKEICKEGCKSFFPVRNTCRMLDIGIPYRLGLSLSN
SSVLNGMDV
>orf02814
(SEQ ID NO: 464)
MDGQLHHRAIFDWVHFENFINSWLGFLTINLKTTGQGAEIVLIGHTFNTRDINLVNLITR
VNHLVCKVPIIGQNQDTRCIPVQTTHRVNTFFDIGQEVDNRLATLVICYTGNDTAWFVKQ
IIDLFFVVDRLTFNFDLVA
>orf02820
(SEQ ID NO: 465)
MHKLRIFVNQLYRRFGIILGPFLVLGFQVLTQELELAIFFDLREEVLLQVIPQVCHFCYL
RKEFTTLNQHELTSHDHVLTRHFQTHGLQG
>orf02831
(SEQ ID NO: 466)
MLHMNLFFQPFFTNLCKTLATGCCVKTVMEWSSIATTIDFKIIE
>orf02832
(SEQ ID NO: 467)
LDNRAKEWIMSTAQNQAIHLSNQGTQGFIDHLLGNTG
>orf02843
(SEQ ID NO: 468)
LSNFCFKTVTVHRYSVNTNVNQNFSTISCFQTKSVPCWKGN
>orf02853
(SEQ ID NO: 469)
MKIKEQTRKLAAGCSKHCFEVVDETDEVSNHTYGKATLTWFEEIFE
>orf02858
(SEQ ID NO: 470)
MSIVKSHSFSISLGIFNSFWNNIHTSECFYFLCKGKSNRSNSTISVNQMVFFINIQRFYC
FAIEDFCLLRI
>orf02859
(SEQ ID NO: 471)
LNTLLPPDNLCLFTIYLTGFSCICINSYCHNFWEIFNQLFYQLS
>orf02865
(SEQ ID NO: 472)
MIDKVIEQYKSHDNLDIRVELHKKYSKNKLGFNNWIFSNYQITDEVKVLELGCGTGELWK
SNSDSIDKMKQLIVTDFSKDMVKSTKSVIGNRNNVNYEIMDIQKISFENETFDIVIASML
LHHVNDIPKALSEVNRVLKTGGIFYCATFGENGVVNYLASLFKDEVNQDLENRTFTLQNG
KRYLSRYFNSVDTLLYDDELQVTSIDDLVKYIQSFKGISEIGSLEEEIIRKRLESEFNNG
MLIIPKEYGMFIARKES
>orf02867
(SEQ ID NO: 473)
MDLGFDYFGSALTISPHKNSQTINSIGIDVQKIYTPHYLPNDFKKNQGYKRSVEMCEEYD
IYRQCYCGCVYAAQAQNIDLV
>orf02868
(SEQ ID NO: 474)
LTKYADVTIYFANSNIHPKAEYHKRVYVTKKFVSDFNERTGNTVQYLEAPYEPN
>orf02876
(SEQ ID NO: 475)
MYNKVILIGRLTSTPELHKTNNDKSVARATIAVNRRYKDQNGEREADFVNMVLWGRLAET
LASYATKGSLISVDGELRTRRFEKNGQMNYVTEVLVTGFQLLESRAQRAMRENNAGQDLA
DLVLEEEELPF
>orf02880
(SEQ ID NO: 476)
MQFTRTAHHTKTLFTTKFTWENEIPFWHHSSRKRDNGFQPHTRIGSSCNDLYSLITCDCN
LADVEVVTIWMGYHLNNFTDNKLRFLIINNFFCKTFRLQLLVQTSDLLICQKDLTALCNF
K
>orf02885
(SEQ ID NO: 477)
MSYSVDDVVSNAFKKRMILDSFFAFNCSGTMKVSTWVYDKGEWYYVSSSGAMIANDWVKD
NGK
>orf02895
(SEQ ID NO: 478)
LTSFCFKANMFNNCLRICRIAEGHILKLDLTFEVFISQLHLNRVLDRRMQI
>orf02897
(SEQ ID NO: 479)
LINPLSRDHSSGKNDEECSHEKEAHDNLHSIRHENNHVTKERQTRYRSSVVNHIGPNPVN
RHTQTT
>orf02900
(SEQ ID NO: 480)
LHLRTCFVRQTNKLSPLINRTRLQFHQTILHYTLNQITSNRLGNIEFLLDIFNQDQVLVF
LAIIQKMHNLTLRPTHKFNAATFGFLLHHQVNLMTKTLKD
>orf02916
(SEQ ID NO: 481)
MLEIWKYRPFVSEFWNDEKNNHDKQFVDPISLYLTLKDDDDPRIEEESEALENMILQYLG
EDDAS
>orf02918
(SEQ ID NO: 482)
LTLFFFELLICLLNSEFDLSKFIFVYFDEYFHEDSLKMNLHQFSFSF
>orf02924
(SEQ ID NO: 483)
MAFNQFNRCIGLSIPTAPNVPGTIINRSYLHDATVPNNVREKT
>orf02940
(SEQ ID NO: 484)
LRLQIELTWFEEIFEEYKFEIMKIRQTGGCFVSHLTERDGLRVT
>orf02944
(SEQ ID NO: 485)
MKKNRGIQKLAILVLLGVFMFSNTIPYQQFIQKNRQLEIRVQSQKKSNGLDVGKAD
>orf02946
(SEQ ID NO: 486)
MKKLFILISNLLASLFFVWVLTIWTDTYVSHYYPNVVVRDSSPETTFQHVATRLEKLAEE
TDSFIAIQHQDPNSEGTTVFSYTTFGDGKLPDGLQEKNLEDAQSSSVETNYFVFDGHLDI
HLLREELSQLGLTNMHLTIPSKLSTLMAIFSNGFQLISLLIFILTFVALTLISQISQLRS
SGIRLISGEKRWSIFLRPVGEDLKAIAVGFSLAGVLAILMQKILSLPTQSLMTIGEGLLS
YNLILLSISLFFAQLFAVGIKKIHLMQIIKGQVPVRGIISLILIGQLLAIIIVTLGIGSS
LKYSQAWQQHRIGQEIWSQERQLITLSISREGTSPGFDEQAQRKLRTWYQLMDLAVSEQK
AFLSRHQLIDRTLQNGMASSKNLITSTEWHDYNPNGNVLIVTPQYLERQNIPVDTTIEQK
MNHLNVGEFVLLLPEHLRSEEEHYKSVFEDDLTSRISSQDERQQMTATVGYLESGQDRFV
YNTTPISYQQFLKDPIIIVITPQSTGPQSILFWIDAVQNYVLFNQLSDAQELIQRQGIEN
WVSEMQTGYHNYITLLDNIQRERWVMLAGAVLGIATSILLFNTMNRLYFEEFRRAIFIKR
IAGLRFLEIHRTYLFAQLGVFLLGFVASVFLQVEIGVAFLVLLLFTGLSLLQLHVQMQKE
NKMSILVLKGG
>orf02955
(SEQ ID NO: 487)
VLKWCILRINHHISRKVDNFLEGTRAHIKGQAHTAWNPLEVPDVRYRSFQFDMSHTLTTN
FRTRYFNPTAVTNNSSVTNTFVLTTSTFPVFCRTKDHFIKESFTFWFQGTIIDCFRFFDF
SIRP
>orf02962
(SEQ ID NO: 488)
LVDPLVTSHDNLLSKGSIFIQTRVSLSYSIFIFFISCQPNNFRS
>orf02966
(SEQ ID NO: 489)
MPWKELCHKLAPKVFKVIRIYSRENKKSPSNWAFCSFET
>orf02970
(SEQ ID NO: 490)
VSVLFFCSYFSLSLEKGWFSSLISCKFMNQFLPFCWRQDSPWILTLAQDSITYH
>orf02978
(SEQ ID NO: 491)
VTDENTRKVRSLVAFFSIVIGYILSSFFISLYHLWQEALRGLL
>orf02979
(SEQ ID NO: 492)
MRLLFFFANRVIRSKENSSTCPSRSYNLLINTSNVSHITIAVNGTCTGNNTTITKIWVSY
LSIDS
>orf02983
(SEQ ID NO: 493)
VDSLFLSLGEEGNQEINLQESFSSTDCNPTLISPETTVAQGLCQDIIYRPFT
>orf02985
(SEQ ID NO: 494)
VNPKSLGSFFLQDSKGFKELVLGHAKLSLPRIVHNVCPQFKNASRIITTRDDFWNACYSL
QMFNIFKGIQVNGRTQFTCIGVFLVWRVVGREHNLRTQKVQFMAHQKLYITRAVHTTTFF
LENFQNSWSWSSLNCKIFLKALVPRKSLVDGSCLLTNPLLIIQVKGSRELGNNRF
>orf02989
(SEQ ID NO: 495)
MTTIRSLLLNFISISYSIFIQKIKKQTRKLVAGGSKHCF
>orf03003
(SEQ ID NO: 496)
MDALVLQKNQETIQQIAVKIRFLDGHDYYSLIDIDNRRTNQTVFPFVNFEDIAF
>orf03004
(SEQ ID NO: 497)
MAFFTEIPTRACLINLAITLHIVETCQGFNDLSLHLRVLAL
>orf03008
(SEQ ID NO: 498)
MLLPLPFNTSKIKQIAMHSDLNQKEMIGHIFHDEDIF
>orf03013
(SEQ ID NO: 499)
MKQTVKKLALVASIAATLGGSVAVASAAVQYPEGGVWTYGSGNGGAYSNYYHPSKYHSST
VVSRKTGSSDKGYAGAGGTSRAWIRTSWGEKVAFYYNV
>orf03018
(SEQ ID NO: 500)
MLNRRFIKTNNIHLCHTHLSSQGNFFCLTTCKFFYIQVCMCIKNHLF
>orf03029
(SEQ ID NO: 501)
MNTIERTRRLVKGCATHCFEVVDRTDEVSSKHCFEVADETDEVSSKHCFEVADETDEVSS
KHVFEVVDETDEVSSKHVFEVVDETDEVSSKHVFEVVDETDEVSNHTYGKATLTWFAEIF
EEY
>orf03033
(SEQ ID NO: 502)
MLERLKSIHYMFWASLIFMLFPILPVVIGELPAWHLLVDILFVVTYLGVLITKNQRLSWL
FWGLMLVYVAGNTAFVAGNYIWFFFFLSNLLIYHFGVRSLKSLHVWTFLLAQVLVVGRLL
IFQRIEVEFLVYMLVILTFVDLMTLGSVRIRLVEDLKEAQVEQNTQINLLLAENERNRIG
QDLHDSLGHTFAMLSVKTDLALQLFQMQAYPQVEKELREIQQISKESMCEVRTIVENLKS
RTLTSELETVKKMLEIAGIEVETDNQLDTASLTQELDSMASMILLELVTNIIKHAKASKA
YLKLERTEKELILTVSDDGCGFAFLKGDELHTVRDRVFPFSGEVSVISQKHPTEVQVRLP
YNERN
>orf03036
(SEQ ID NO: 503)
MTVVKVEKLSKKIKDKEILRNISFEINDGECVALIGPNGAGKTTLLDCLLGDKLVTSGQV
SIQGLPVTSSKLDYTRAYLPQENVIVQKLKVKELIAFFQRIYPNPLSNQEIDQLLQFVKQ
QKEQLAEKLSGGQKRLFSFVLTLIGRPKIVFLDEPTASMDTSTRQRFWEIVQELKAQGVT
ILYSSHYIEEVEHTADRILLLNKGELIRDTTPLAMRSEEIEKHFILPIAYKEVVEQSNLV
ENWTLKQDSLQVVTREADAFWELLAQAGCRMQEIEVNNRSLLNTIFEETQKGDN
>orf03039
(SEQ ID NO: 504)
MGEEEMRNKMIIAMSLVVTGVMTYLMFSGLDEDFCHFPWKVFAGFGIMS
>orf03040
(SEQ ID NO: 505)
LVEQLTFNQWVTGSSPVRVIYAGLAELADAPDLGSGA
>orf03063
(SEQ ID NO: 506)
MPSLRSLKKTDGSCDELHHFDLPVNFFKNTVLGKQTCSGVIREVCQDCFNMLWR
>orf03091
(SEQ ID NO: 507)
LHTSFRSSVGHSHTWHQDIVRPILFSRFNDSIVILWQNCPTFNQGIYCYLDCFFPIVSL
>orf03096
(SEQ ID NO: 508)
MWSQTLGLIHPLTSLLELPFWMACLKGFGQFCKSLSGLLSFVAECQHLLSLCSRFIRITV
LQTSKV
>orf03112
(SEQ ID NO: 509)
MLEQARLKVEQQAIKNIQFLEQDLPKNPLEKEFDCLAVSRVLHHMPDLDAALSLFHQHLK
EDGKLIIADFTKTEANHHGFDLAELENKLIEHGFSSVHSQILYSAEDLFQGNHSEFFLIV
AQKSLA
>orf03113
(SEQ ID NO: 510)
MKHDFNHKAETFDFPKNIFLANLVCQAAEKQIDLLSDKEILDFGGGTGLLALPLTPSQAG
>orf03117
(SEQ ID NO: 511)
MVDLQSFFTRKYLNLNSVDAYLILPRLQGHLSYPQDFFLLQDFCFLLPIFLNLSQKEGRN
AGKDS
>orf03133
(SEQ ID NO: 512)
MRIRNSPFDHILQTIFEFEDRTCQVTCRFEACSSICNDNWEFSQHIISVFQSPSCHTVCD
KSDVFCSFLFDKNFASLWIYVVTITDQLCXXIPFIN
>orf03142
(SEQ ID NO: 513)
MHKTCLNIWKFLFYQIESLIHQMAADKSPCRIGNRGR
>orf03144
(SEQ ID NO: 514)
LNHRFNRQTTKVGRSTIWANGTVNRLIIFVIRSTCIVLINGHSFRCQTSSSTSLPNTKDK
VRLITIHLFFQYLSRFVKNCRHL
>orf03147
(SEQ ID NO: 515)
MTLHQTFRFQNFEMPCQSSLINFQTLLNRHLVTRRMLQQKQ
>orf03151
(SEQ ID NO: 516)
MKKRMLLASTVALSFAPVLATQAEEVLWTARSVEQIQNDLTKTDNKTSYTVQYGDTLSTI
AEALGVDVTVLANLNKITNMDLIFPETVLTTTVNEAEEVTEVEIQTPQADSSEEVTTATA
DLTTNQVTVDDQTVQVADLSQPIAEAPKEVASSSEVTKTVIASEEVAPSTGTSVPEEQTA
ETTRPVEEATPQETTPAEKQETQASPQAALAVEATTTSSEAKEVASSNGATAAVSTYQSE
ETKVISTTYEAPAAPDYAGLAVAKSENAGLQPQTAAFKEEIANLFGITSFSGYRPGDSGD
HGKGLAIDFMVPERSELGDKIAEYAIQNMASRGISYIIWKQRFYAPFDSKYGPANTWNPM
PDRGSVTENHYDHVHVSMNG
>orf03156
(SEQ ID NO: 517)
MSNQITVHHSHEHLQKVFTHSWQCNIKNVFIFLKQSLLLMKRNSVGFPTEFTPSILKS
>orf03171
(SEQ ID NO: 518)
MAELNSVITTVTGIENRLGAVILAEIRNIHAFDNPAQLQAFAGLDSSIYQSGQIDLAGRM
VKRGSPHLR
>orf03178
(SEQ ID NO: 519)
MEXXXDIRKGRHAVVEKVMGAQTYIPNTIQMAEDTSIQLITGPNMSGKSTYMRQLAMTAV
MAQLGSYVPAESAHLPIFDAIFTRIGAADDLVSGQSTFMVEMMEANNAISHATKNSLILF
DELGRGTATYDGMALAQSIIEYIHEHIGAKTLFATHYHELTSLESSLQHLVNVHVATLEQ
DGQVTFLHKIEPGPADKSYGIHVAKIAGLPADLLARADKILTQLENQGTESPPPMRQTSA
VTEQISLFDRAEEHPILAELAKLDVYNMTPMQVMNVLVELKQKL
>orf03191
(SEQ ID NO: 520)
LTNLSSVDSEELFQFYRERGNAENFIKERKAGFFGDKTDSSTMIKNEIRMMMGCLAYNLY
LELKQLAGDEVKALTIKRFRRLFLHIAGKYVSTARRHILKFSSLYAYSKQFQALFDTICQ
INLILPVPYRARGQGKTCLTE
>orf03203
(SEQ ID NO: 521)
LFDDRQAINICPPTNGSLRLTSLQVDQNPCPPSTNLNKILARSQFLNHIQQISLSLELLQ
ANLWNLV
>orf03207
(SEQ ID NO: 522)
LSVHFCSSHRCLLVRYNDTYSTKKGLKFETFLSVFRYDFLGM
>orf03237
(SEQ ID NO: 523)
MDFFNYLLWMICHNHGLHTLLLSKDCVCHTARDKDGNHRIKSVFPTKGQTCYQHDSSIYQ
ERNTTDILTRFLANSQADDIRPTTGDIVSKSKTNPQTHNNTPKKGIDNGILRQGCHRDKL
DKEGTHRYRDKGKDGELMANLIPS
>orf03245
(SEQ ID NO: 524)
MKIKEQTRKLAAGCSKHCFEVVDETDEVSSKHGFEVVDETDEVSSKHGFEVVDETDEVSN
HTYGKATLTWFEEIFEEH
>orf03253
(SEQ ID NO: 525)
MKIKEQTRKLAAGCSKHCFEVVDRTDEVSSKHGFEVVDETDEVSNHTYGKVKLTWFEEIL
EEY
>orf03254
(SEQ ID NO: 526)
LFFKDEKQALYTKPKTKSSSFRASKVSNQTIVATTRTDCQVIALNLCDKLENGVVVVVQT
THHIGIDDVIYSKIFQHLTHSIKMSLAFFIKKVQDRRRILYCHLVFFFLRVQDTKRIFLQ
ATLAILRQGLLERCQIVNQGLAVGCTALRISKSVEVQFDTLNTDFLQKMGCHNDCFHIGS
WIARTKTLNTNLVELAQAPCLWTLITEHRSHVVELAWLLHFWGEEFIFHIGTDNGRSSFW
TEGNMTVTLVIKIVHFLGYDIRCISDRATDNLVMLKNRRAHFCIVIALENLTGKALNVLP
LSRFSR
>orf03260
(SEQ ID NO: 527)
MEQIGKVFRQLRESRNISLRQATGGQFSPSMLSRFETGQSELSVEKFLFALENISASVEE
ILFLARGFQYDTDSELRKEITDVLEPKNIAPLEDLYRREYQKHAHSHNKQKHILNAIMIK
SYMKSMDERVELTAEEGKVLHDYLFSTEIWGIYELNLFSVSSAFLSVSLFTRYVREMVRK
SDFLMEMSGNRNFFHTILLNGFLASIECEEFTNAYYFKRVIEEHEYKENETYFRIVYLWA
EGLLDSKQGRVKEGQKKMEDAVRIFEMLGCNKSAEYYRNTTEC
>orf03261
(SEQ ID NO: 528)
LIPYFLHFIIFFRKFIKNLPNCQNYEKIEDIYHVEGLL
>orf03263
(SEQ ID NO: 529)
MKIKGQTRKLAAGCSKHCFEVMDRTDEVSSKHCFEVVDRTDEVSNHTYGKATLT
>orf03266
(SEQ ID NO: 530)
VVPFSDTFKDRNQVDIFTIKISRCNSSTIGENSWDIHISNSNHRSRHVLVTATDSDEGIH
VVTTHSRLDGVRDDVTRC
>orf03275
(SEQ ID NO: 531)
MKKFSYPTRQTGEGVKYQSQMVRQWFLIRIFRLFSVA
>orf03297
(SEQ ID NO: 532)
VVLDHQNQLFEARFLEHTNPLTRIQLTRVKALRILLSSPPFLVIKGIRTEVDKSC
>orf03305
(SEQ ID NO: 533)
VQKLKKAIYKAHLKDSDDFRPETSTPNLFESCLKLCPCFLSS
>orf03306
(SEQ ID NO: 534)
MGALGYYEGFVPYVSNQYKNQAEEEDKPLSDKYIFEKILGKTYAAFKKDQINERVEKLGK
LKPITINYNGKSEVIDSKEKLQELMNKAVKDEVAQI
>orf03307
(SEQ ID NO: 535)
MMGDGMKEFQFERKQRFSLRKYAIGACSVLLGTSLFFAGMGAQPVQDTETSSALISSHYL
DEQDLSEKLKSELQWFELENKLLNLWEH
>orf03308
(SEQ ID NO: 536)
MKIKEQTRKLATGCSKHRFEVVDKTDEVSSKHCFEVADRTDEVSNIYTARRR
>orf03312
(SEQ ID NO: 537)
VNITKTSIIKAHTTKEDGIDHTFTRFNIMSIFYSTRKIFLDKLNSTNRQFLGYIISTRCY
QSFNSVSQSIHTSSSSQAFRFGKHEFRVINRDKSKAILVNHYHLNLAFFISNHIVNSDFC
RSSCRCIDSHNWQAFFSRLMKPFIILWFSTICSHDRNTTSCILWRTPAKTDDKVTAMFLQ
SSYPICDIFTSRVWLYIAKDDIFDSFCIQWF
>orf03318
(SEQ ID NO: 538)
MSQDEKLIREQICDVCHKMWQLGWVAANDGNVSVRLDEDTILATPTGISKSFITPEKLVK
LNLKGEILEAEGDYCPSSEIKMHIRCYEEREDVRSVVHAHPPIATGFALAHIPLDTYSLI
ESAIVVGAIPITPFGVPSTMEVPEAITPYLPDHDVMLLENHGALTVGSDVITAYYRMETL
ELVAKTTFHGRMLLSTKGIEEQEIARPTLERLFSMRENYKVTGRHPGYRKYNGDGSMKET
EK
>orf03320
(SEQ ID NO: 539)
MESKKIAKQILIATAVLTSFLGSNLVYADVVQSNSNNRASTETARVTGNNLEKLITKDKE
IDKEMTYLSDMDWSSATHGDIDKTKTVQKDAPFTTGNKGEHTKISLLTSDDKVKYFDKGI
GTVADSPSVISYDISGQGFEKFETYIGIDQSANSSRSDHAVVDRIEIEIDGKVVYSSSVT
NPEGFRYNTQAQFISVTIPQNAKKISLKSFAGEHTWGDEVVFADAKLIKTVSTQTITPDL
LNKGINGGVYLSDLEWVDATHGDDDKSKTVQKDKPFTPGNNGSNNKIKLLIDGKEVEFNK
GLGTVASNPSSIKYDVSGANVTRFISYVGIDRSANHLNSDYADIQKFEVVADGKVIYSSD
SKYPKGIKYDTSAFLVDVEIPKDTQTIELKSYSGKHTWADELVLGGALFMANGKFKNPND
WSEVDKRREINNEHPLLMMPLYANGEEFNQGKYTFWGGDTLTGKWENIPDDLKPYTVIQL
HPDDLPKRDGAARDFYEHMLEEAAKYVNPKTGKNEPIPVILTVYTAGNMPYYTSAHWLST
SWIDKMYQKYPNLHGIFSTENYWIWANDIENKAADYLKVSAKNGGYFIWAEQNNGSAIEK
AFGKNGKIAFQKSVDKYWKNLIFMFKNTPAAEGNDSTTESYMKGLWLSNHTYQWGGLMDT
WKWYETGKWKLFASGNIGKSQGDRQWLTEPESMLGEEALGVYLNGGVVYNFEHPAYTYGV
NNKESLLFSEVIKEFFRYVIAHPAPSKEKVLEDTKVFIHGDYSNKGNGKFFVNVNTDREQ
TPLYMTGRYNVIPAIPGVLKTDKLKESVSSSRIQIKEITSPEFSSTQARKEYLNKLYPMN
YEGDIFAQKLDNRWFVYNYKVNENVKQTGKLKFNSLEMNVEFEPHTYGIFERISNGLKVN
LNNFRTNKDSLWSNAQDANQAKKLPQLTKKGAIKWIEEHYIKDTQFGEKRVTKIVLRGID
KLPTIHSLSGTNNSYDQPSLNFDQKNHMVTITINSNGNLEFELHF
>orf03322
(SEQ ID NO: 540)
MTIYINKDETVFHLAMKDSSYIFRILENGELQHLHFGKRIHVKENYNQLMAYKKRGFEVS
FSEEFEDIQQSMIQNEYSSYGKGDFRHPAFQVQGMNGSRITTLKYQGFELEKGKNRLNSL
PSTFDDIGQCAETLTIILTDSILDLTVRLNYTIFPEYNVLVRNTEFLNNSNNKLTLLKAM
SLQLDLPDSQYDFIQFSGAWLRERQLYRTSLRPGIQAIDSLRYSSSPQQNPFFMLSRRET
TEHSGEVYGFNFIYSGNFQNMIEVDHFDTARVTVGINPVEFRFLLNPAESFVTPEAIVIY
SDQGMNQMSQQLSDFYRHHLVNPNFSQASRPIILNSWETFYFDLGTEKILDLAKAAKDLG
IELFVLDDGWFGHRKDDKSSLGDWVTDRSRLPEGIGFLADEIHKIGLQFGLWFEPEMISI
DSDLYKNHADWTIHLLDREKSVGRNQYVLDLTRQEVVDYLFDSISKIIIKTNLDYIKWDM
NRHITDIYSIELDSEQQMEFGHRYILGLYQLLDRLITKFPSVLFESCSSGGGRFDLGLMY
YAPQAWTSDDTDPIERLKIQHGTSYGYSPSMMTAHVSISPNEQSGRQTSLDTRTNVAYFS
SFGYELDVTRLSVEEKEQVREQIQFYKKYRSLFQYGDFYRINSPFSCDSASWQVVSKDKC
QSILLYAQLNSKLNPGYTRVYFSGLDKDKCYSVSRFDEFFYGDELMNAGIKVSLSNLALC
VPEYLTKLFVIEEVVCKY
>orf03323
(SEQ ID NO: 541)
MKIENKNVRRNFFWGEGRFYTTDIVNKRAGVMIKNVSKEEFTITLENGIKLSSTHFSAIV
REEGDTRIQVSFVCPSIRLRLIFESRDDVLSKQLVLESSTEVIKSVEVESFEFETEDNIF
YPKRQDCIKEMANFSGYYVELGQPVYANSLFLGMEFPMSENKVDGRHYVSRYYLGTVVNQ
EKSLWSCIIGGACSYKKEEIQEAFFEYVEGIAQPSYFRKQYNSWYDHMTDITEEGILKSF
SEIRDGFENHGVHLDAYVVDDGWTNYQSVWEFNHKFPNGLRNIKYLVNGFGSSLGLWIGP
RGGYNGTEIIMSDWLEAHPELNIGSKNLISNDVNVADFNYLNQMKKKMLEYQKEFDISYW
KIDGWLLQPDKPDKSGPHGMYTMTAVYEFLIQLLIDLRKERGGKDCWLNLTSYVNPSPWF
LQWVNSLWIQISQDVGFTENAGNDINRMITYRDSQYQEFLEKREIQLPMWSLYNHEPIYA
VSANTWYMDHQMFASIPDFEAYLLFISTRGNAFWEFHYSFDMFDEERWKANARAVKWIEE
NYQTLKYSKKIGGSPEKFEIYGYKCHNQKTSTEILSLRNPAQIKQKIKIENLSIENFTRV
IGDFTIQEDEIELAPYSIVILKK
>orf03324
(SEQ ID NO: 542)
MKHTLETINSRIQWFREARFGMFIHWGLYSIPGKGEWIRSHQKLSIEDYEPYFRAFDPKE
YNPREWAKQAKAAGMKYMVLTAKHHDGFCLFDSKFTDYKATNTPAGRDLVKEFVDAVRAE
GLKVGLYFSLIDWHHPDFPKYADLNHPMRGNEVYRDEKINFDSYLEYLHNQVKEIVTGYG
QIDILWFDYSYEDMVGEKWGASKLIDMVRHYQPNVIVDNRLETSGEGFGSIVTDEITSYA
GDFVSPEQIVPHEGIRNFKGEPVPWELCLTMNNNWAYNPTDYLYKSSQTLIRKLVECVSK
NGNMILNVGPDALGRINDSSKKILDNFHRWMSRNGEAIYGCSGDENLPKPDWGYYTRNGN
TVYAHVFEQPIGPLALLGISKENVKRMSFLHDGSEVKISESWTTNAYKGICFAQFGEVPH
FTYPLPDLIDSVIKIELRE
>orf03325
(SEQ ID NO: 543)
MNTHINGISKKGKVLIYGYMLLTILISIFPIAWIFLSSLKADPMKNPGISLPTDFTLEGY
INVFTKLHVFTYFWNSFKVVSISVIISIVMISMSSYVIARMEFRGKKLVTSMLYSTLFIP
ATAMTFPVYRLVNELGIYNTPVALILVYSCSGIAMSFFIIKNYFEIIPKELEEAAEIDGA
TYAQTFWKVMLPIARPGILTAAVLAFINNWNEYYWASMLVIDKNELTVPALLGQFTTSFN
TNYNGLFSAIVVIVLPPIILFAFTSKYFIEALGGGAVKG
>orf03326
(SEQ ID NO: 544)
MAQKIMSLQNRKNQKRRFIFLFLLPTLICFFLFYFYSVVTIFLTSFAKWDYTNLNTPEFL
GFDKLFENYRYVFKEYPFFTEALINSVRWAVIGVIIQVPLAVSVAITLSKKLKGWKISRN
LYIVPSIISSAAMGLIFLQIYNPNYGVVNQIIHLFNPSFKDSVLLTPGLNIVAMTGAYIF
FAGASTIMILGQIFAIPEEVQEAAILDNITGWRKEWYITIPMIKGTIKTVSIMAATSGFL
LYNEVFFLTNGAAGTKSISFVIRELAVASSRTQYARANTIGVIQILGGMLIIVCINILFR
ERKRLKGEK
>orf03327
(SEQ ID NO: 545)
MNKKSLLKCAVIGLVATFGLAACGTSKDASGGSSSGKEVLEFYHGYHHSEDEWPVAKTMR
DLYDKFAEEHKDSGVEFKPTPVNGDLKDIMNNKVASGEFPDVIDLAGNAVSLAAIEQKLV
LDLKPYIDSNKLEKNVGLNYKQNQKDGKIYTVHEQLFTMGLWYNKDIFAKAGAKTPDQWN
TWDDFTQAMASIRKQDGVYAFGAGEPSIRLFNTVLGTTENGRKLLDKPLTKEGIESKEFA
DALKMVMKEIQANGSKNAGGDANAYSKDFQEGKSAVFFNGVWASGEMSKNPSLAPGIYPA
GVAISSSGGGITISSKMSEAKQKLALEFLKYMTSDDVQKVIFEKVGANPSNENVNVKELS
EKSSEATTKILGQAITQVKNAKAVVPTVSDVWGGDVHTAIINALTESAAENVDVDQKVKS
TQDVLKSLIG
>orf03337
(SEQ ID NO: 546)
MNIAIRIILNFFRVMGNHQNSLAMMMGAVVHEFVKFIFTSCIHPRCRLV
>orf03338
(SEQ ID NO: 547)
MLLIMSIQTTEPAFSRIATRLDKFIDRTWKTSIKTGNLLRKIGYSQFLTLRICL
>orf03339
(SEQ ID NO: 548)
LQNSKTGLDERRLSRSIFPSQGNKFPTINTIIDMFKNRLLIIIEGQILYRNISHYLISPT
KAVKNR
>orf03344
(SEQ ID NO: 549)
LTSLIPRLMFQKTSQLVSIKILLEGCWIIAIFTEPLR
>orf03352
(SEQ ID NO: 550)
MSNSFVKLLVSQLFANLADIFFRVTIIANIYIISKSVIATSLVPILIGISSFVASLLVPL
VTKRLALNRVLSLSQFGKTILLAILVGMFTVMQSVAPLVTYLFVVAISILDGFAAPVSYA
IVPRYATDLGKANSALSMTGEAAQLIGWGLGGLLFATIGLLPTTFIILVLYIISSFLMLF
LPNAEVEVLESETNLEILLKGWKLVARNPRLRLFVSANLLEIFSNTIWVSSIILVFVTEL
LNKTESYWGYSNTAYSIGIIISGLIAFRLSEKFLAAKWESILFPLVAMAIVTLTILYFPN
AQMFLLFSALVGMLSQLKEVPESVFLQETVEENHLVNVYSVLEVISTLAFSVFVLLMSYI
TDFGYQPFV
>orf03353
(SEQ ID NO: 551)
MSKLLDKILSRENMLEAYNQVKSNKGSAGIDGMTIEEMDNYLRQNWRLTKELIKQRKYKP
QPVLRVEIPKPDGGIRQLGIPTVMDRMIQQAIVQVISPICEPHFSDTSYGFRPNRSCEKA
IMKFLEYLNDGYEWIVDIDLEKFFDTVPQDRLMSLVHNIIEDGDTESLIRKYLHSGIIIN
GQRHKTLVGTPQGGNLSPLLSNVMLNELDKELEKRGLRFVRYADDCVITVGSEAAAKRVM
YSASRFIEKRLGLKVNMTKAKITRPGELKYLGFGFWKSSDGWKSRPHQDSVRRFKLKLKK
LTQRKWSIDLTRRIEQLNLSIRGWINYFSLGNMKRIVASIDERLRTRLRVIIWKQWKKKS
RRLWGLLKLGVPKWIADKVSGWGDHYQLVAQKSVLKRAISKPVLEKRGLVSCLDYYLERH
ALKVS
>orf03357
(SEQ ID NO: 552)
MFLRCATFKLADSRLNIFTCFFFGEIRFNSRNQVVKAFITDGTVISTIIVRGTVPCNQWT
KTCPAAFDIINGDVGFWKAVVDNAK
>orf03358
(SEQ ID NO: 553)
MLQITCVVCISCTKVSLVFTWENKDHTTVTQTCVKVNWL
>orf03359
(SEQ ID NO: 554)
LRSLIRQITYFITPRTCCINNQTGLDFKYLVCQEITSYNTCNLATFVKEEAFCLHVVGNE
GTVLVGTFDVFNHETRIVVTEVKIHSTSYQAFLLQVWLAFQDLILAQNLVRSWCVAHTC
>orf03360
(SEQ ID NO: 555)
LHFNQTSLKTASCRLQGYTSSCDSSTDNQEVQGAFLHFFN
>orf03375
(SEQ ID NO: 556)
LIHSKMFVKSIRLRKKRVWDKKILILGILYYKFLKSID
>orf03379
(SEQ ID NO: 557)
MFASKSERKVHYSIRKFSIGVASVAVASLVMGSVVHATENEGSTQAATFSNMANKSQTEQ
GEINIERDKAKTAVSEYKEKKVSEIYTKLERDRHKDTVDLVNKLQEIKNEYLNKIVESTS
KIEIQGLITTSRSKLDEAVSKYKKAPSSSSSSGSSTKPETPQPETSKPEVKPEPETPKPE
VKPEPETPKPEVKPEPETPKPEVKPEPETPKPEVKPEPETPKPEVKPEPETPKPEVKPEP
ETPKPEVKPEPETPKPEVKPEPETPKPEVKPEPETPKPEVKPEPETPKPEVKPEPETPKP
EVKPEPETPKPEVKPEPETPKPEVKPEPETPKPEVKPDNSKPQADDKKPSTPNNLSKDKQ
SSNQASTNENKKQGPATNKPKKSLPSTGSISNLALEIAGLLTLAGATILAKKRMK
>orf03384
(SEQ ID NO: 558)
MDREILKFFQDLLSILSHNDMITLFCQKCCNSFSNHFLVICN
>orf03386
(SEQ ID NO: 559)
MFITLRRICLRACVVEKEQSYLKFLFFQKRPVSFLHVKSVLAGI
>orf03387
(SEQ ID NO: 560)
MVKTTDRLEAIGFSFILFENLFKPCQLYLQPQNSVLSNLQLAA
>orf03393
(SEQ ID NO: 561)
MTRKLNPSYTNVASATTLTFNQVASTFRKACLDHVVNLTRNNLKGICQLTPLQLHDTRLI
>orf03421
(SEQ ID NO: 562)
VKAPIPKAPLAHSFGSASIIAHTIHQKFNLKVPNYRQEEDWTKMGLPITRKEISNWHIKT
SQYYLEPLYNLLRERLLTQPLLHADETSYRVLESDSQLTYYWTFLSGKAEKQGITLYHHV
LIDLFISYFNPL
>orf03430
(SEQ ID NO: 563)
LVSVFYSLLQVDNVDSVTFSKDVLSHLRIPATSLVTKVYTSLKKLFH
>orf03440
(SEQ ID NO: 564)
LIVWILKNHTDLTTYIPNIFLSQTLAINYNLSGFCFQ
>orf03441
(SEQ ID NO: 565)
MPYNRKPFSTFHVKRNILHIVVVLIFFIAKRKIFYINY
>orf03445
(SEQ ID NO: 566)
MFKKMSNSSRILFYISVNFCDKRIYRTKLYSDTPVNLFKFLFRQKSNCQSVGQTSSINHF
FYSWIVFFFKNNLCHSIPSIK
>orf03458
(SEQ ID NO: 567)
LADGSGKLAEGGTKLTSGLEDLQTGLASLGQGLGNASDQLKSVSTESKNAEILSNPLNLS
KTDNDQVPVNGIAIAPYMISVALFFAAISTNMIFAKLPSGRHPESRWAWLKS
>orf03464
(SEQ ID NO: 568)
MKNTVKLEQFVALKEKDLQKIKGGEMRLSKFFRDFILQRKK
>orf03483
(SEQ ID NO: 569)
LVEQLTFNQWVTGSSPVRVIYAGLAELADAPDLGSGA
>orf03487
(SEQ ID NO: 570)
LWVNIDHIRHISCENACLQYFTSIWYIDDFDLDLRVFLLKITSDFFQGFGHFFFLVEILD
GHCVIRRFTIVGASAEPKQS
>orf03496
(SEQ ID NO: 571)
MKIKEQTRKLAAGCSKHCFEVVDETDEVSNHTYGKATLTWFEEIFE
>orf03504
(SEQ ID NO: 572)
LIDVLFINSFIGRICFYCYRRIHATCLFLQLFSIVILNVAHTLKHSIFIVITFISRCRNF
IIVRILLENQFSRNQGIDNRVGQSRY
>orf03505
(SEQ ID NO: 573)
MINVNQVSIEVKNTFKNWNFTSSIELTTFSKFSQSPTMT
>orf03516
(SEQ ID NO: 574)
MGFSMKLIHDLDMHTTHSTAKMLYNVKAIKNDFSIRE
>orf03521
(SEQ ID NO: 575)
MYQDLLRKIAEEKPNYNQEEIQWLLDHLGDPSPEIRDDLVFTSFARGIQEELFTQEQFHF
IAEGVSSDGGLDKEIDKIGLPTLERSFRALIYATLLSDDANQQSVFYQELNAGFRNDLSN
QGLHYLSKEKDTTGFSSQYGWVHSFAHGADLLTEVVCHPEFPKNRVHEVFDILGQLFKRI
SIRFTDDEDWRLARVIYEPILQGKLEQEQVASWIKTVDFPIEAREDFYKFSNFRSCLGKS
XXIHSLIN
>orf03524
(SEQ ID NO: 576)
MGFKVSHFKIPSSHLSINVLRTIENFTEIGQGLLHISP
>orf03525
(SEQ ID NO: 577)
VGFFDFGLTNSCRQVRQFTQTVQDFLVCYHQGIVKEGQGYAGICFKFHPSLGNIGKFVIA
IVRRLRHKSIVANMAHLNVDLFQFRKGLLEILKSVKIALVITAKLVDVFTSFLDCTQEIL
TVLV
>orf03537
(SEQ ID NO: 578)
LKKAQWGFSNQGPDGLFLVRPTSNRDEIPPRDLAHPQNQLFLSFSQVEKLAWHAPSPLSE
FALNISLPLSSPPYLWPFIQSPVLYCASHFSIHFNSFSSHLRLVITISPHLLSSTSLLLH
TLCAQHTIHSTDLHHLRTPPPSGLFPLALYTRLAAPTLTYHTLSNIQSLKQQLVXXFIH
>orf03548
(SEQ ID NO: 579)
MANDNKSHYLIYRVLGISFEEGENIDLYQNKGRFLYKYAGSFLEEAAVLSFNEKFGTENT
>orf03553
(SEQ ID NO: 580)
MVNAMHFSFSILIEGNSRKVGICLLNRTHTRFKLSQAIYL
>orf03562
(SEQ ID NO: 581)
MSQQLSDFYRHHLVNPNFSQASRPIILNSWETFYFDLGTEKILDLAKAAKDLGIELFVLD
DGWFGHRKDDKSSLGDWVTDRSRLPEGIGFLADEIHKIGLQFGLWFEPEMISIDSDLYKN
HADWTIHLLDREKSVGRNQYVLDLTRQEVVDYLFDSISKIIIKTNLDYIKWDMNRHITDI
YSIELDSEQQMEFGHRYILGLYQLLDRLITKFPSY
>orf03572
(SEQ ID NO: 582)
VKEEKKAIVLGADNAYMDKVETTLKSLCVHHYNLKFYVFNDDLPREWFQLMEKRLETLNS
EIVNV
>orf03574
(SEQ ID NO: 583)
MKIKEQTRKLAAGCSKHCFEVVDRTDEVSSKHRFEVVDRTDEVSSKHRFEVADRTDEVSS
KHRFEVADRTDEVSSKHRFEVADRTDEVSSKHRFEVADRTDEVSSKHRFEVADRTDEVSN
IYTARRR
>orf03576
(SEQ ID NO: 584)
MEXXXFKAGAKFFWAKLGLESLEAKEILRDGGWDDIVKNRCIKREQPRLIEEA
>orf03588
(SEQ ID NO: 585)
MILLQNKSFCYSISQKETSLCHANVVTLNLHEALLNQISDNFSIKSSKRLSKFFLKSRHG
NPGFLAESQEHFFFHLLLLTQVIFCNTSIAFLNRTEIMTNGTMIFIAYSIDITRCPCTNT
IVFSIVPVHEIMTTFKAGFGEIRNLIMLKTRSLQLCNDVLKHLRF
>orf03612
(SEQ ID NO: 586)
MDREILKFFQDLLSILSHNDMITLFCQKCCNSFSNHFLVICN
>orf03614
(SEQ ID NO: 587)
MEDHLLINAVDEFRSISFFQFFKHAFFHIFLVKTNILSPKTNSLIITKGSRTTCFFFACQ
IRSSWYLKNQTNIQ
>orf03620
(SEQ ID NO: 588)
LFIRKKHTWINLTPKQTIFSQMNAQFFINFTRRALKRTFITFTSPTWQFPHIGPGNACLI
IT
>orf03637
(SEQ ID NO: 589)
LNIAYTDNPAHIFGKIFHDNLLALLIIFDDVTCKACLRQDKVNSGLFLDSLFNSGCKGVG
FSLVRLVISI
>orf03641
(SEQ ID NO: 590)
MCELDILHDSLYQFCPELHLKRLNSLTLACHALLDCKTLTLTELGRNLPTKARTKHNIKR
IDRLLGNRHLHKERLAVYRWHASFICSGNTMPIVLVDWSDIREQKRLMVLRASVALHGRS
VTLYEKAFPLSEQCSKKAHDQFLADLASILPSNTTPLIVSDAGFKVPWYKSVEKLGWYWL
SRVRGKVQYADLGAENWKPISNLHDMSSSHSKTLGYKRLIKSNPISCQILLYKSRSKGRK
NQRSTRTHCHHPSPKIYSASAKEPWVLATNLPVEIRTPKQLVNIYSKRMQIEETFRDLKS
PAYGLGLRHSRTSSSERFDIMLLIALMLQLTCWLAGVHAQKQGWDKHFQANTVRNRNVLS
TVRLGMEVLRHSGYTITREDLLVAATLLAQNLFTHGYALGKL
>orf03644
(SEQ ID NO: 591)
MXXTKSSCLITTGRNDSPSTCLPRVASNNDRFSSEFRIIPDFHCSKKGIHVNMDDFS
>orf03650
(SEQ ID NO: 592)
MHFHIIKLVNHFQLLIKLNRISHPNLHIKSSFLSLVLLFYQKEQDFAIMVI
>orf03660
(SEQ ID NO: 593)
MDRGESLSDCVCMAGYEPANSSRLSIEGTYENKLYKLISSKYHTTGNDIMVCVPCGYTKY
KETPGPHACTSCPGRTHAASTTNTNQDQCNRCPPGYYETNDPSYPCDVCSPNHICVGSDP
MDPALMLYSGKRIKCDKNSVTLVPFEENVHLASCLCDKGYMARTRTGIVKCEAVPKNTYK
DVVGNVGPTNCPPGSYTLKIGATDVSECVCKRGMFFDKDNKRCTVCPVGMYCLGGRLPNG
EHMLPMMCTDGNAVTKDGGATSPGECLCKPGFYLRQDGPGGCVECPENTYKSFISNENCS
PCPRIL
>orf03667
(SEQ ID NO: 594)
MXXAFCLPVFAHPETLVKVKDAEDQLGARVGYIELDLNSGKILESLRPEERFPMMSTFKV
LLCGAVLSRIDAGQEQLGRRIHYSQNDLVEYSPVTEKHLTDGMTVRELCSAAITMSDNTA
ANLLLATIGGPKELTAFLHNMGDHVTRLDRWEPELNEAIPNDERDTTMPVAMATTLRKLL
TGELLTLASRQQLIDWMEADKVAGPLLRSALPAGWFIADKSGAGERGSRGIIAALGPDGK
PSRIVVIYTTGSQATMDERNRQIAEIGASLIKHW
>orf03668
(SEQ ID NO: 595)
MARFIRSQTLTLLEKLNELDADEQADICESLHDHADELYRSCLARFGDDGENL
>orf03669
(SEQ ID NO: 596)
MTVRELCSAAITMSDNTAANLLLTTIGGPKELTAFLHNMGDHVTRLDRWEPELNEAIPND
ERDTTMPVAMATTLRKLLTGELLTLASRQQLIDWMEADKVAGSLLRSALPAGWFIADKSG
AGERGSRGIIAALGPDGKPSRIVVIYTTGSQATMDERNRQIAEIGASLIKHW
>orf03673
(SEQ ID NO: 597)
MKIHKTVNPVAYENTYYLEGDKHLIVVDPGSHWEAIRQTIEKINKPICAILLTPAHYDHI
MSLDLVRETFGNPPVYIAESETQLAPKPLPNNPLGPPXHSFIN

In some embodiments, preferred OXC141 antigens are selected from the polypeptides orf00045 (SEQ ID NO: 53), orf00068 (SEQ ID NO: 65), orf00074 (SEQ ID NO: 70), orf00223 (SEQ ID NO: 99), orf00229 (SEQ ID NO: 104), orf00360 (SEQ ID NO: 117), orf00506 (SEQ ID NO: 135), orf00781 (SEQ ID NO: 177), orf00785 (SEQ ID NO: 178), orf01068 (SEQ ID NO: 198), orf01446 (SEQ ID NO: 235), orf01447 (SEQ ID NO: 236), orf01449 (SEQ ID NO: 237), orf01455 (SEQ ID NO: 242), orf01460 (SEQ ID NO: 247), orf01461 (SEQ ID NO: 248), orf01463 (SEQ ID NO: 250), orf01464 (SEQ ID NO: 251), orf01466 (SEQ ID NO: 252), orf01467 (SEQ ID NO: 253), orf02661 (SEQ ID NO: 433), orf02690 (SEQ ID NO: 439), orf02698 (SEQ ID NO: 444), orf03318 (SEQ ID NO: 538), orf03320 (SEQ ID NO: 539), orf03322 (SEQ ID NO: 540), orf03323 (SEQ ID NO: 541), orf03324 (SEQ ID NO: 542), orf03325 (SEQ ID NO: 543), orf03326 (SEQ ID NO: 544), orf03327 (SEQ ID NO: 545), orf03562 (SEQ ID NO: 581), orf03660 (SEQ ID NO: 593), and immunogenic fragments thereof.

6. Sequences Identified from INV200
>orf00004
(SEQ ID NO: 598)
LKGVDDFLFIFEEGFKQGGKARADRDYSGVSSLRNSSKVYLEFLY
>orf00005
(SEQ ID NO: 599)
LCSALKNSYDIELIKVLSNKAHLYLPIETVTPQTVSTS
>orf00006
(SEQ ID NO: 600)
MRVAETSIVKKNHQIPCIINQKIAQKLIKKTSMTDIDHQLSISTSTVIRKINDFHFEHDF
SRLPEIMS
>orf00010
(SEQ ID NO: 601)
MFKSNLSLSQSLPHKDFFFFKRIIHLFSLFLLIDFIIIS
>orf00015
(SEQ ID NO: 602)
VEEVEVAEVKNARVSLTGEKTKPMKLAEVTSINVNRTKTEMEEFNRVLGGGVVPGSLVLI
GGDPGIGKSTLLLQVSTQLSQVGTVLYISGEESAQQIKLRAERLGDIDSEFYLYAETNMQ
SVRAEVERIQPDFLIIDSIQTIMSPEISGVQGSVSQVREVTAELMQLAKTNNIAIFIVGH
VTKEGTLAGPRMLEHMVDTVLYFEGERHHTFRILRAVKNRFGSTNEIGIFEMQSGGLVEV
LNPSQVFLEERLDGATGSSIVVTMEGTRPILAEVQALVTPTMFGNAKRTTTGLDFNRASL
IMAVLEKRAGLLLQNQDAYLKSAGGVKLDEPAIDLAVAVAIASSYKDKPTNPQECFVGEL
GLTGEIRRVNRIEQRINEAAKLGFTKIYVPKNSLTGITLPKEIQVIGVTTIQEVLKKVFA
>orf00018
(SEQ ID NO: 603)
MGVSIFLALFYMIPALYFLFRIGKKWELPKKVLILSLLGGMFLSGWLSSFANTYIHDFMN
CTPKVRQKKSNFWGVLL
>orf00019
(SEQ ID NO: 604)
MKLSYEDKVQIYELRKQGQSFKQLSKRFGVDVSGLKYMVKLIDRYGIEIVKKGKNRHYSS
KLKQEMMDKALLEGCSQRSISLDYALPNQGMLSFWPAQYKKNGYTIVEKTRGRPAKMGRK
RKKTWEEMTELERLQEENERLRTEVAYLKKLKELEERDEALERERQRQLEKWFQEDFD
>orf00020
(SEQ ID NO: 605)
MVSGGFRLDFLLETARLARSTYYYQLKQLDGVDKDKEIKTEIQGIDNEHKGNYGYRRIHL
ELRNRGFVVNHKKVQRLMRILGLTARIRRKRKYSSYQGEIGKKAENLIQRQFEASRPMEK
CYTDVTEFAIPNSTQKLYLSPVLDGFNSEIIAYHLSTSPNLEQVKSMLEQAFTEKYYENT
ILHSDQGWQYQHDSYHRFLESKGIQASMSRKGNSPDNSMMESFFGILKSEMFYGYEKNFR
SLENLEQAIVDYIDYYNNKRIKVKLKGLSSVQYRTKSFG
>orf00024
(SEQ ID NO: 606)
VNIATLQNGHILGWQIQHIANKLTSNFWIAKDFLSYQVIGWANARMTYSHISSLFIISQF
>orf00026
(SEQ ID NO: 607)
VSITFSLTNFFKILINLTAQVSPQVIDEKILMMDLNLNNYLSTVIQLRQDVYTGIKILHR
VRHGE
>orf00027
(SEQ ID NO: 608)
MSRYSYSLDSRKIVFEISCFKEKKASLTLFFHLFESSIMKLATQPSYSSFYSELK
>orf00033
(SEQ ID NO: 609)
MKIKEQTRKLAAGCSKHCFEVVDRTDEVSSKHRFEVADRTDEVSSKHRFEVADRTDEVSS
KHRFEVADRTDEVSSKHRFEVADRTDEVSSKHRFEVADRTDEVSSKHRFEVADRTDEVSN
IYLRQGDVDVVEEIFEEY
>orf00034
(SEQ ID NO: 610)
MKPGAEGWKDERSQDIEEKDNGDGLGYFLFLSMDNRRCRCNGRTPTDRRTYSNQGSQFCI
QGKEALEEVGNNQGY
>orf00035
(SEQ ID NO: 611)
LPNCEDLRDIETKTKQDNGILEQFLGTKGQSNIQLFIRREKGM
>orf00044
(SEQ ID NO: 612)
LSLLDLRGSLCLRIYLHEPLITTVSQDFTSLSDISHF
>orf00047
(SEQ ID NO: 613)
MDFKSFIIGLVVGIFGPYMDDLIRKKFLKSSEKKTEKSVKK
>orf00093
(SEQ ID NO: 614)
MTYEYKSHIYLAETVLNVKDLASQTTFYQQVIGLEILSQTETESILGLGGKVLVQLIQAQ
ESGEVREHXXXFHSLIN
>orf00103
(SEQ ID NO: 615)
MVSNLVFIGNCNFHNTVIFHLLNRLNQGPLQILSQNHDKGRRLSWIFKSRLGQLNASKNW
MGRKEQAMALAIAADLQDQLLFKKAD
>orf00113
(SEQ ID NO: 616)
MKFNPNQRYTRWSIRRLSVGVASVVVASGFFVLVGQPSSVRADGLNPTPGQVLPEETSGT
KEGDLSEKPGDTVLTQAKPEGVTGNTNSLPTPTERTEVSEETSPSSLDTLFEKDEEAQKN
PELTDVLKETVDTADVDGTQASPAETTPEQVKGGVKENTKDSIDVPAAYLEKAEGKGPFT
AGVNQVIPYELFAGDGMLTRLLLKASDNAPWSDNGTAKNPALPPLEGLTKGKYFYEVDLN
GNTVGKQGQALIDQLRANGTQTYKATVKVYGNKDGKADLTNLVATKNVDININGLVAKET
VEKAVKDNVKDSIDVPAAYLEKAKGEGPFTAGVNHVIPYELFAGDGMLTRLLLKASDKAP
WSDNGEAKNPALSPLGENVKTKGQYFYQVALDGNVAGKEKQALIDQFRANGTQTYSATVN
VYGNKDGKPDLDNIVATKKVTININGLISKETVQKAVADNVKDSIDVPAAYLEKAKGEGP
FTAGVNHVIPYELFAGDGMLTRLLLKASDKAPWSDNGDAKNPALSPLGENVKTKGQYFYQ
LALDGNVAGKEKQALIDQFRANGTQTYSATVNVYGNKDGKPDLDNIVATKKVTININGLI
SKETVQKAVADNVKDSIDVPAAYLEKAKGEGPFTAGVNHVIPYELFAGDGMLTRLLLKAS
DKAPWSDNGDAKNPALSPLGENVKTKGQYFYQLALDGNVAGKEKQALIDQFRANGTQTYS
ATVNVYGNKDGKPDLDNIVATKKVTININGLISKETVQKAVADNVKDSIDVPAAYLEKAK
GEGPFTAGVNHVIPYELFAGDGMLTRLLLKASDKAPWSDNGEAKNPALSPLGENVKTKGQ
YFYQVALDGNVAGKEKQALIDQFRANGTQTYSATVNVYGNKDGKPDLDNIVATKKVTIKI
NVKETSDTANGSLSPSNSGSGVTPMNHNHATGTTDSMPADTMTSSTNTMAGENMAASANK
MSDTMMSEDKAMLPNTGETQTSMASIGFLGLALAGLLGGLGLKNKKEEN
>orf00118
(SEQ ID NO: 617)
MSLQIKLKKLAKELSKLLKDSNLETVDKDVLENSQKELQKAVLFLADEKGSEHTEAEVID
NLKEVIAKLKANA
>orf00129
(SEQ ID NO: 618)
VGRFFGSSQTSDEFFFSFDSSIVKELSEIVHGFDTVSFR
>orf00140
(SEQ ID NO: 619)
MKIKEQTRKLAAGCSKHCFEVADRTDEVSSKHCFKVVDGTDEVSSKHCFEVVDRTDEVSS
KHCFEVVDRTDEVSNHIRQGDVDVV
>orf00146
(SEQ ID NO: 620)
MNDDDSRCIHIERDGKTIEFGYLNISSTDRNTSHADGLVGIFNSNFSGVRVRGIAVFLNG
PDNLDTTLVGNFQTIWNFRIICIHS
>orf00147
(SEQ ID NO: 621)
LEFNFCRSIIKNGRDNLPNTNSTSGMATRWANHNWSDDIKDRLKTK
>orf00152
(SEQ ID NO: 622)
MSCNCAFYRSQFFDVNSVSNYHSHQKELRFPNSILFTYFVKVA
>orf00156
(SEQ ID NO: 623)
MSKEKVILAYSGGLDTSVAITWLKKDYDVVAVCMDVGEGKDLDFIHDKALKVGAVESYVI
DVKDEFATDYVLVALQSHAYYEQKYPLVSALSRPLISKKLVEIAHQIGATTIAHGCTGKG
NDQVEYQIAVAKKANEAKK
>orf00157
(SEQ ID NO: 624)
MRYDFGKVYKEIRESKGLTQEEVCGGVLSRTSLSKIESGKTTPKYENMEFLLRQINMSFE
EFEYICQLYQPSQRTEIMQTYLNMRSIIGTSDLVNLFQKCQDYLKTHHDLPIEEIRDMLE
VVIYIRQHGAGELSDHAEQVVKKLWRKIEKQDTWYESDLKILNTILFSFPIEYLHLITGK
ILQRLEVYKNYQHLYDLRIAILLNLSTLYLYNQDKNMCKQICYTLLEDAKNKKSYDRLAI
CYVRIGICTDDSKLIQKGFSLLELTEETSMLSHLKKEVEIYYQAKER
>orf00158
(SEQ ID NO: 625)
MKIREIIGTDMYGTTVSGIVSGLNKLNFTVKAVRVALEDLTPKLTFPAILQVKNDLGQNH
FVVLHSIKEKINGTRITK
>orf00159
(SEQ ID NO: 626)
MELVLPNNYVVIDEEEMMYLDGGAIYIPRWAITGAITGAAYAALAAAGGGGLQLVLASYG
LRSALVAGIVKGLGVLGIHIGNAFANTVIRSIASAGIGAGADWIFTNIIDGWDGRRDNQL
RIG
>orf00161
(SEQ ID NO: 627)
MATITNALNIAATVAEVFSLGGAIAYGLDIVDGKFDGYLWA
>orf00162
(SEQ ID NO: 628)
MKDDQKYLLAGLYSLLVAIFYFPLIESKGIFVSILMAVLLLYLIYFIATVIHIVIIKFIR
KKSFKYLVLYPFTYDGSWRFQPINLLYFPEMVRDVIPINLVQEYCQGQPYGLLKKMLKRI
RLSREIALLLATIIVYFFTHRILPLSVFTFMFSYILLFVQSYLGSNTAWIGNRRLIIDDE
FEKILLSKSYIKEISSARYSEYLTCEYKNPTPIILIAIFENLLDSYLLQNQSEVDLDIFY
KVLPLLYKEKYTMGFNYFVSLNYLLYKVGFLGIIYDNEALRDLSKQYLNKNISELQDGSF
EGGIQDAVASKQIVVINEFIACLNSKCMPSQYDRFFYKDRPYIFSRKSSIKG
>orf00163
(SEQ ID NO: 629)
MKNKRYFFDTILIILLLISTIFCVSPVFIKLDILGTPSHAILTFVLAIPLFYILSQCLHT
LLLLVSSIFCKLRPIYFYFIFVIIIGARKYYRILFHQLMGFSPGIAVFYKESQTTKNLFK
FYYFLYFTTLISYYFFFTFVYDKSLLLPLIPFSIIIALVQKLYRIENQQLFLLKSKVLTI
LESKRDCEFNLQDYHEIWKLQSKSELPCVALSYISLIKPYLSESVREQIDLLEVKRFKKI
NHPISLYGMLDVIKLNLYLRHYNEKNKYESMLNKILEVRPDFVLIEQNIDDSLNSSQPLS
LSLAISEIQLLLEVYIGIKHVSIRR
>orf00164
(SEQ ID NO: 630)
MIRKPIIFLLMLPIWGLWIELHLLVSNLQLNLEIPFDFVVSTSLTFFVLILSKIVLDILY
ALKDLYKKEALITIFPFIFIGRKKVNVRFSPYFSFHRKSLSPDDLRSRIIWSFILEIAII
LVFILKIPFAIIMLTTIFFWTIMDINHLVFNKTEFLFNQNKWQKEDSFESDLTKTLKDKI
QKSELSYSDLMSLQLYDAMNQSTFLTDSELFEDILKKIEDSHNTLLCTGLVELLLYEISI
SNNNNWQEKVDKIRIQLIRINQLDFFYYTSWLRQNFDFCMNREYHKMKSRKLLLSNKKIV
>orf00165
(SEQ ID NO: 631)
MELVLPNNYVVIDEEEMMYFDGGAYLSKRACQGICAALAMSSGTFIALAGAAVLTKKLIN
YIKVGGLGGWLIGAAAGKIAYYIGYGVLNRGCDINGNPYPWDGFISATVR
>orf00166
(SEQ ID NO: 632)
MSNVDKIRKIHIIVCWVYIFLSFRAIINDTEYFLLIFLAFIYSIVSLPLYSVKNKIVSIC
LAINSILLMSFPILINKFFPESFLTYTVLISVFITELIIFHLIGKDFDIKLTNEYKKISQ
FRSKVSQSPWIKYLEISSFILTIFPSILYGTVDNHVLTLIFLIKICVDTTIKFLFIRLFD
TSTLMKRRIFFLFALDVIAYLFLGYLLVIQKAGYLFSVLLLFSNFSVPFIKEKEYELFKN
SK
>orf00167
(SEQ ID NO: 633)
LFNEIKKTSSLIGNVFIGMKEDDAMFKKRIEKGKSSVFIFLE
>orf00168
(SEQ ID NO: 634)
MNKKKMILTSLASVAILGAGFVTSQPTFVRAEEAPVASQSKAEKDYDTAKRDAENAKKAL
EEAKRAQKKYEDDQKKTEEKAKEEKQASEAEQKANLQYQLKLREYIQKTGDRSKIQKEME
EAEKKHKNAKAEFDKVRGKVIPSAEELKETRRKAEEAKAKEAELTKKVEEAEKKVTEAKQ
KLDAERAKEVALQAKIAELENQVHRLETELKEIDESDSEDYVKEGLRVPLQSELDVKQAK
LSKLEELSDKIDELDAEIAKLEKDVEDFKNSDGEYSALYLEAAEKDLVAKKAELEKTEAD
LKKAVNEPEKPAEEPENPAPAPKPAPAPQPEKPAPAPAPKPEKSADQQAEEDYARRSEEE
YNRLTQQQPPKAEKPAPAPVPKPEQPAPAPKTGWKQENGMWYFYNTDGSMATGWLQNNGS
WYYLNSNGAMATGWLQYNGSWYYLNANGAMATGWAKVNGSWYYLNANGAMATGWLQYNGS
WYYLNASGAMATGWAKVNGSWYYLNANGSMATGWLQYNGSWYYLNANGAMATGWAKVNGS
WYYLNANGSMATGWVKDGDTWYYLEASGAMKASQWFKVSDKWYYVNGLGALAVNTTVDGY
EVNANGEWV
>orf00190
(SEQ ID NO: 635)
LKKRMNRWQFLLNQSKEMVGILLLKMKEQELIEFVVNL
>orf00191
(SEQ ID NO: 636)
LIKVIKRKAFGFRNFNNFKKRILMTLNIKKESTNFVLSRL
>orf00195
(SEQ ID NO: 637)
MTYNEKRLTNSLERVHMEQLKNTTDLLGLEDKNIKILSVLKYQTHLVVQAKLDSPAPPCP
HCQGKMIKYDFQKASKIPLLDCQGLPTVLHLKKRRFQCKNCLKVVVSQTSIVKKNCQISN
MVRQKIAQLLLEKQSMTEIAHRLAVSTSTVIRKLREFKFETDWTKLPKVMSWDEYSFKKS
KMSFIAQDFESKSILAILDGRTHAVIRNHFQRYQREVRELVEVITMDMYSPYYRLAKQLF
PKAKIVLDRFHIV
>orf00196
(SEQ ID NO: 638)
MGYSLKKSRTYCEQDPEKVNRFLKELNHLSYLTPIYIYETGVETYFYLEYDRALSRQLVS
LEEDIII
>orf00201
(SEQ ID NO: 639)
MRFYCEAYEVITEEKNVYFNDIELEVKYSSVEELFIICEKLLDKKKVSFFYVDEKPLRYL
LFDYIFLLVLAKKNIPILDGVSNKQVDPTLLHHFSLEIEKNFIDFCYKNMDLILKTQSIS
LCHREELIIVDVSDDSKFGVFYKRFRTLVDKNNGKYVCVYNFSRVSDILQNWKNYCNRKF
SVTFTESQFELFKLLYNQKNFKTISLLFGKKIVAGGIIYYSDLTNIEYFCIFWWDSMFGK
DSIGKYVYVEEISRCHFLDRNYSFCYGLQDYKSKLIKYFLE
>orf00202
(SEQ ID NO: 640)
METRNLISYSLTDIFETDKIRIELLGEIYYKNIKLELHEFAGLYKIYGISLIKNITGMFL
IIIFDTKTKELKIFQDITTSYFNLYYTVYGGVFYYSTSLKKVMKLSHVPVTLNNKKIQEF
MRNGFILDSNTLVTEINKLEYFSYISVNNTLRICGIDYNDSNNFTKEQVLKNWDSMLRES
ILRVYSEAGEANITLSSGFDSNYILYTLANYTNSSINAFCIGGEKGINEIPEVTKIAKFY
GINLLVDTVMSQDLEYFPDIVWRLEGSLFECGVILQYYLGRLLFQKGNTSILCGESADEI
MTFKYHSVNYNQFCNDKQKSVYFSYSDYPFYVTNSIVLKKNSLLLHSFGIHPRYPYKMSE
IVEMSKKISDLNDKKEFHKKNCELRFQDSVLDNINSVPGTTHLFSCLNIQTLVKIILYIF
RYNSCMKIFNFRDKELIFFNKIVNGLIQNIEENLEDDIERILKYLYICLFNEIFIIKNKV
NFFDDVEFNQTLSEFLDKL
>orf00204
(SEQ ID NO: 641)
MKFFCENNLNITFFDTFSEIKNNIDYYIIALPTDYDEKIGSFNTYEIEQTVSKILRVKPN
GKIILKSTVPIGFSNKLKRLFDTKNIIFVPEFLREGCSIYDNLYPSRIVVGDETVEGRKI
AELFLSISTHSTANIKNVMLVSPTEAEAIKLFSNTFLALRVAFFNELDSFAERRSLNAEV
VIKGVCLDPRIGNFYNNPSFGFGGYCLPKDTKQLKKEFIEINAPVIEAIDISNTNRKQFI
VKQILERKPKIVGIYKLGMKYNSDNYKESAILSIINELLIVGIKILVYEPNLNVSIDNVI
FEKNFELFTKQSDLIVANRWDRGLEAYKDKVYTRGIWIRD
>orf00206
(SEQ ID NO: 642)
LEEESFIMENTEFSLELDVTEVATEQDYVSSGVISTGCCKN
>orf00207
(SEQ ID NO: 643)
MFKIKDNYIYRQCVNDSILIEKLNENNLEIFFDSKIFQEMLMVANPRFFNELTKEKIYQN
STFRNYAKRSLTRATPFGLFSSVGVGSFSKVSYPQQIRENYSKKVSVSGEWISSLCMMLE
NEDSVLLQLHLQWNQKVLELSDKYQLNNINYWGVSEQSRDILIKKTALLEFIKKLTYKSE
VSVLDLVQEIQTKSPNLETQKIIDYLRNLIISEFLFTNLRKVVINHNCLDNLIYILSSIN
EQTKLTTDLLQLKSCIEKYSKSELGEGILQYAEICEKMSHIFNEEKQRYLKVDLVNSYDS
LLPKDLKKTLEDFVNFISRINLGKDYRNKELISYTEKFVEKYGEYVEVPIKQLLDSKLGL
GIPKQNLEPYSILSSVAEQTFLSYLSKEIFKAVKNNKKEIDISNIPPELLYPNLDRFAVN
QFELYCEMKNFGEQPVISIVPNTGSDMIGKSIGRFASYFLNSNIELDSRVDNVELIEFPS
DNKNLNVMSSHHGHSKKLLLSYEDDFDIDSLELDFLVVGVERVNEHYKLYFRDLRTDLIV
NFVTTSMLNHKSIGVFSHLARFLLTVSLEWQDNPFSLFRVIENLDFLPYIPRIKYKNIIL
SEEKWILSDVDKKDMSTISQWKKFFDVPSLLYFHKDDERLLIDLKNSLDVQWILKQNVDK
LHFTRFDKIDGKNCEFIFGFENPRNSVYPHSVSEKTVRRIENDFYKDYVKTFSSDWIYFK
LYGINSSTMPELRENLLIFTDELLAENLVSDFHFVNYNDGGDGSIRLRFKIMNEDDFEKL
RYRIIHWIDFLLNHYFCKDVSFNLYEREVERYGGIGFLTVCERIFSIDSYLVLKLFSKKV
LKVDDYLSVLHSIFIYIRLLGISPKQLLKLMKDTFTQNIYRKSFKKVFPNNAKVIKEFKQ
YFEDQSKFDIFNEVFKSFSPIEKHFEYKNDMIHSLLHMHMNRIGIFSLNEKEYLYFVRYI
LEVLNNYEKYN
>orf00208
(SEQ ID NO: 644)
MRNIIKKYDEIINKVDSLVLDNNIIDLLQRSCYTENRSYLSEYPSIIIYLSYRLANCDDN
EHSKLLYNRVNYYLHELLKSIKLNSRNNISMCYGFSGYVYALKLLPKRSKEYSKLLETLE
TILVSLTRDRLSEIKKSNKVKEEYIDVIQGVSSVGKYFLSKDKLTSNQELLLKGVLNYLA
GVINNKPTIYPEYMPNEKLKRKFPNGYINLGVAHGILGPLYVLALGFKKFNMPEYLISLK
KGLSYYEKTFQTNKIGKIIGWNGRVSAEVESEKFEYNLSWCYGSLGMARVLYNISKIIDI
PKLQELATDVFHSSIYYLNSSEILNNAICHGRSGIMLLFNLMYLDTGESQFKAISDNLFK
EIVNKATDSEYIFVERDIYFRGVNYDEVIEYIDFCLLNGVSGIVLALMAQRTGNASPLAE
MFFMQ
>orf00209
(SEQ ID NO: 645)
MKKILNNKLYMKVLVSDLISNFGDTLYFIALMTYVTEIKSSNLAISIVNISETIPILFTI
FFGIIADRTLNKVGMIIKTLWIRTILYLLVAVVMNFKESILVVILASIVNLISDTLGQFE
NGLFYPISNRIVKKSDREETMAFRQTATSTMNIVNQSLGAFLITFLSFFHLALINSLTFA
ISLLITLAIKSQINNFYIDKTPSTKVSKVDFKATFSDIISNLKLSLKHLFSLTNMKTVLL
VIPILNGSLAIIIPLAVVNLSKSSALTIISSATTISVLGISTVSGGILGGTLILISKKFK
NLSIENLLKMNLMTILLSFIAFYYQNIYFIVLTLFLSSVFVSALNPKIGAIIFNNLDETK
LATIFGGMVTYFQLGDVVSRLLFSTLVIYLSYTYIAVIYMILVLIVAIYTFRRVQTTS
>orf00213
(SEQ ID NO: 646)
MKIKEQTRKLAAGCSKHCFEVVDRTDEVSSKHCFEVVDRTDEVSNHTYGKVKLTWFEESF
EEYK
>orf00217
(SEQ ID NO: 647)
LKSSILSKMGDFSVRYCNLVGTVLFGVVLIAILRLVF
>orf00246
(SEQ ID NO: 648)
MGLDVGSKTVGVAISDPLGFTAQGLEIIQINEEQGQFGFDRVKELVDTYKVERFVVGLPK
NMNNTSGPRVEASQAYGAKLEEFFGLPVDYQDERLTTVAAERMLIEQADISRNKRKXXIP
FIN
>orf00270
(SEQ ID NO: 649)
LNTSYSFGKKDQFALEHCFCIKLSIFARAVTLFVSCIN
>orf00291
(SEQ ID NO: 650)
MLIGEGYRTFPVLIYTQFISEVGGNSAFAIMAIIIALAIFLIQKHIANRYSFSMNLLHPI
EPKKTTKGKMAAIYATVYGIIFISVLPQIYLIYTSFLKTSGMVFVKGYSPNSYKVAFNRM
GSAIFNTIRIPLIALVLVVLFATFISYLAVRKRNLFTNLIDSLSMVPYIVPGTVLGIAFI
SSFNTGLFGSGFLMITGTAFILIMSLSVRRLPYTIRSSVASLQQIAPSIEEAAESLGSSR
LNIFAKITTPMMLSGIISGAILSWVTMISKLSTSILLYNVKTRTMTVAIYTEILRGNYGV
AAALSTILTVLTVGSLLLFMKISKSNSITL
>orf00292
(SEQ ID NO: 651)
LIIIASMSAPFVGAYSWVLLLGRNEVITKFLTNALYLPAIDIY
>orf00293
(SEQ ID NO: 652)
MERKKLNIWTVSSFFLFLTYPIFLVYPIVTVLKQALIHEGQFSLANFVTFFSKAY
>orf00295
(SEQ ID NO: 653)
LLSTTEFIGLSIRILSNLHEFKILVGLLNQFFFWNLLLHKTKSNVVSDSQMWENSVVLEN
HPDIAFAGFHIIDFCIIEVKFSTFDTVETCNHTKKGRFPTS
>orf00314
(SEQ ID NO: 654)
MITIKKQEIVKLEDVLHLYQAVGWTNYTHQPEMLEQALSHSLVIYLALDGDAVVGLIRLV
GDGFSSVLVQDLIVLPIYQRQGIGSALMKEALEDYKDAYQVQLVTEETERTLGFYRSMGF
EILSTYNCIGMTWMNRKK
>orf00325
(SEQ ID NO: 655)
MKIKEQTRKLAAGCSKQCFEVVDRTNEVSNHTYGKATLTWFEEIFEEYNTNLEYKQPICS
QEKA
>orf00359
(SEQ ID NO: 656)
MVDNIPKRVNDVIRQAGNNAKTSRPHVGIGKSHISVSFLFPYHTANRIKNQEKVIF
>orf00375
(SEQ ID NO: 657)
LFDLLDHGLDTVLVCHVTDISMGLDANFTISFNPFIDQILIDIVKDNSSAGFSVGFGNSK
SNSIRSAGDESNFSF
>orf00387
(SEQ ID NO: 658)
MKSLARLLIIHVFISIFLFFALTSGAISHTVLLLLLLFLPALNKGLEKIQSKRIPVLNAA
LFFLLISFPQLLTNPVQWKFSIFLVVTIISSLAYFYNFYQVVKEVDQKQLI
>orf00390
(SEQ ID NO: 659)
LEAAGEIETEFQGWIVLVVFNHIDSLSRDTDILGEFELGNTQFLAKFFHTIHLISFLIYV
VYI
>orf00403
(SEQ ID NO: 660)
MEGVNHVDIIKVSCCSFISQVNWMMKGKIPNREGFKFSVARFDAIDLVVVHIGHTRCQFS
RTGSRSGYDNQVATGFDVVVFAHAFWGNDVIHIRRISFDWIMKIRINSVFLKLVAEGICS
GLASVLCNDNGTNKNP
>orf00404
(SEQ ID NO: 661)
MFNVASINGNHNLNLLFQFLQELDFVVRFITRKDTSSVEIF
>orf00409
(SEQ ID NO: 662)
MIDIHSHIVFDVDDGPKSREESKALLIESYRQGVRTIVSTSHRRKGMFETPEEKIAENFL
QVREIAKEVADDLVIAYGAEIYYTLDALEKLEKKEIPTLNDSRYALIEFSMHTSYRQIHT
GLSNILMLGITPVIAHIERYDALENNEKRVRELIDMGCYTQINSYHVSKPKFFGEKYKFM
KKRARYFLERDLVHVVASDMHNLDSRPPYMQQAYDIIAKKYGAKKAKELFVDNPRKIIMD
QLI
>orf00410
(SEQ ID NO: 663)
MKEQNTLEIDVLQLSRALWKRKLVILLVAIITSSVAFAYSTFVIKPEFTSTTRIYVVNRD
QGEKSGLTNQDLQAGSYLVKDYREIILSQDVLEEVVSDLKLDLTPKGLANKIKVTVPVDT
RIVSVSVNDRVPEEASRIANSLREVAAQKIISITRVSDVTTLEEARPAISPSSPNIKRNT
LIGFLAGVSGTSVIVFLLEFLNTRVKRPEDIENTLQMTLLGVVPNLSKLK
>orf00413
(SEQ ID NO: 664)
MDKKGLEIFLAVLQSIIVILLVYFLSFVRETELERSSMVILYLLHFFVFYFSSYGNKFFK
RGYLVEFNSTIRYIFFFAIAISVLNFFIAERFSISRRGMVYFLTLEGISLYLLNFLVKKY
WKHVFFNPKNSKKILLLTVTENIEKVLDKLLESDELSWKLVAVSVLDKSDFQHDKIPVIE
KEKIIEFATHEVVDEVFVDLPGESYDIGEIISKFETMGIDVTVNLNAFNKNLGRNKQIHE
IVGLNVVTFSTNFYKTSHVISKRILDICGATIGLILFAIASLVLVPLIRKDGGPAIFAQT
RIGKNGRHFTFYKFRSMRIDAEAIKEQLMDQNTMRGGMFKMDNDPRVTKIGRFIRKTSLD
ELPQFWNVFIGDMSLVGTRPPTVDEYDQYTPEQKRRLSFKPGITGLWQVSGRSKITDFDD
VVKLDVAYIDNWTIWKDIEILLKTVKVVFMRNGAK
>orf00414
(SEQ ID NO: 665)
VTFDKEDARSILENEIFYPCYYPTNRNLKNLIKNTILAFKILRKERPDIIVSSGAAVAVP
FFYLGKIFGAKTVYIEVFDRIDAPTMTGKLVYPVTDRFIVQWEEMKKVYPKAINLGGIF
>orf00415
(SEQ ID NO: 666)
MIFVTVGTHEQQFNRLIKEVDRLKGEGFIQDDVFIQTGYSNYVPKFCKWEKVISYEKMNQ
LIKESDIIITHGGPATFMAVIAKGKNPIIVPRLKKFGEHVNDHQMQFVKITKEIYNLIVI
DDISDLHLILHNFKDKHFETYLNNERFNVRFNVEISNLFHGNKINEN
>orf00416
(SEQ ID NO: 667)
MKIRIEPQYFLYKYLWFIILLPKQFMQLILFFLIALTLLPTYIKEKQVFKIDTPSFCMVL
WTIIYSISIIFNSLIDGLAVQVIFSDLSKAFNWLIAVFFYNYYLKMPINIDRIKRYMYYN
FTILVVFVGLFYIQRGSNVILFGRSLLDWDGFTLATSYGVRYTGFLEYATLNGQLILFLL
PLIRLFRFRFFTQTIIFAFLLEVLVLSKSRIAIVAMLIYIAFAVVNEINSNNKWLIGIFC
PIIPFMLFYNFEKIKQIFFQMFSSRSGSNATRFRVYEESLKAINGMEMLLGAGVRIPSTV
DILLGSHSMYISFIYRTGVLGSIIITVMFYYLFSKFLKCDSSERLRSIGYILALSVFWLF
EELDPHYWCLILFFSTISIFINNRKEEIVG
>orf00417
(SEQ ID NO: 668)
MIEVSIIIPIYNAEKTIKNCVDSALKQNLESLEVILVNDGSNDSTSKILEQYGDNPQVMI
FHQVNMGVSAARNVGLSYASGEYVFFLDSDDILDEGMLSKMYQFAKSNKIDLLSCWHKEP
STTQYGGNDNSSASFIARTKEEIGNHFVDIFPRSACAKLFLRRRIEENNIAFSTEMSLGE
DMSFVCQYLMVSRSIAVIDGLYYTIQNVNPQSLSKRYVSNIENSLLMQNQLWDQLLEVYP
KIEENYYKQHMDFRFYLASLYVNNLFKFDSPYSSKEKWDNIAQQLKKYRPFLDEKVSKEK
KPKNMNEMVIFYLLESKIPALIYSFYSFKEWWKKKRLKN
>orf00418
(SEQ ID NO: 669)
MEDLVSIVVPVYNVEKYLKKSIESILNQTYDNLEVLLVDDGSTDSSGEICDSFIKVDSRI
RVFHKENGGLSDARNFGIEHMKGQYVSFIDGDDYISKDYVWKLYHSLKNNNSEVSICSFS
LVDETGEKIKDELLDSGEVSLSGQQILEKALTADGYRYVVAWNKLYRSTLFEKLKFKKGM
LYEDEFLNYPLFWDCKRVSIVEEPLYLYVQRKGSIIQSNMTLEKIKMKDKMHTSRIEFYA
EKKNSFLHQRSCQQYCNWIVTITVSHYNVLNVAFLKYLQHQFRRIVKYTQNDDKKLIIQN
ILGYINIRLAAYVKSKVM
>orf00419
(SEQ ID NO: 670)
MFPIYIISNQNIAFQQEIDIAYRKMKRQFSHISLTESEQKNDMNISNKVWICWFQGEERP
PELIRTCIQSMRTHFLGREIIVLTEENISDYIDIPDYITDKYKKGSISRAHYSDILRVEL
LCRYGGLWVDVTVLNTGGDFSNLELPLFVYKSLDLSRKDSQAIVASSWLISSYSNHPILL
YARKLLWEYWRRKNSLCNYFLFHIFFTIATELYPIEWSAVLTFNNHSPHMFNFELNNQFS
EKRWEQLKQISVFHKLNHHIDYSIGVNNFYKFIVFSKVEKNE
>orf00420
(SEQ ID NO: 671)
MSNKISKNLAYNIGYQLIGIAFPLITSPYLSRILGAENLGIHSFTISVALYFMMFMLLGI
ANYGNRTIATVKREGKEILSKTFWNIYYVQLLMSVLVTIAYLIYLYFWVSSYKFIAILQL
FLLLSNAVDITWLFYGLEDFKQIVFRNTLVKLLGLFLIFSFVHESSDLWKYTLINGGVTL
VGQLLLWGQLKGRLSWVKIQKKDLLSHIKPILVLFIPVLAISIFSNMDKYMLGLMVGVKQ
VGFYDNANRIIDIPKALIAALEAVMLPRTSYLLAEGQEEKSNYYIEVTILYAMMISSVLI
FGIISVSDIFSLVFWGEEFLESGRLIAAMAPVFVFSVPGNIIRTQYLIPRAKDKDYVLSL
IIGALVNILLNCFLIKPFGAMGATISTVLAEFVLYGVQFWTVRRDLDFKKYLKNGFIFYL
FGMIMYLAIIAVKAHLQYNIINLVLLIVLGGIVYTGFCCFYILISRNVHFEILREKIKRK
IGYENIL
>orf00422
(SEQ ID NO: 672)
MFVADIMISDYSSAPIDFLLLNRVVFLYLPDFKEYQSDKNPFFEVFKVSKTKGIALDPFD
EIIGRFQFGVRIV
>orf00428
(SEQ ID NO: 674)
MGFSMKLIHDLNTHTTHSTAKMLYNVKAIKNDFSIRE
>orf00431
(SEQ ID NO: 675)
MEQLHFITKLLDIKDTNTQIIDVVNRDSHKEIIAKLDYDAPSCPECGSQMKKYDFQKPSK
IPYLETTGMPTRILLRKRRFKCYHCSKMMVAETPLVKKNHQIPRIINQKIAQKLIEKISM
TDIAHQLSISTSTVIRKLNDFHFECNFRNLPKIMSWDVETVRGVTVSIGRWR
>orf00444
(SEQ ID NO: 677)
LQIAQESSQDTDGINPPVVEEAMVFDRNDCLNQICGNIISLGIDAAFRTQVSNELIFIVV
DFTRSCCN
>orf00446
(SEQ ID NO: 678)
MLNLMWMKIFHRNRTFLFCFLDFKVDVISIINARIVRR
>orf00447
(SEQ ID NO: 679)
MYNSQALRQIVVVGSIDHLFKRHSSICEIFGLRKRCLSFL
>orf00472
(SEQ ID NO: 680)
MSLADLLEELEAAKDSKKARSMEAYMRHQFSFLGIAVPERNKLYKNIFQKRKKQRLSIGI
LQTLAGKRSLENTNMWLLTI
>orf00473
(SEQ ID NO: 681)
MEKILLHNLNQTEFFINKAIGWTLRDYSKTNPTWVTCFIEKNKERMAELSIKEASKYL
>orf00477
(SEQ ID NO: 682)
LSTCWNGKFCHICVALFHCFRAFKLALNEILCLLTNVSFIFVSVAF
>orf00487
(SEQ ID NO: 683)
LLGSFFSWTTKELMGIIFFNNFPTVHKNNMMGYISSKTYLIKLIKNSI
>orf00509
(SEQ ID NO: 684)
LKNVFSVGCHFFQFFVRFFWFGKFDHFNLVELVQTDQATRITTGRTSLRTE
>orf00535
(SEQ ID NO: 685)
LIDIKHFFLCLPLSKKMIIDIIVNKNPDRFCMIEKVKKTMAENR
>orf00539
(SEQ ID NO: 686)
VNIDSSEFYISHITDGIFDSFLDSNRYLRNFYSVLKVEIDICCEFFVHVFKINATAE
>orf00540
(SEQ ID NO: 687)
VNTLYLCSSDSNDFFKYTWGDNDFAKLFFNSHRMTSF
>orf00550
(SEQ ID NO: 688)
VKEEKKAIVLGADNAYMDKVETTIKSLCVHHYNLKFYVFNDDLPREWFQLMEKRLETLNS
EIVNV
>orf00551
(SEQ ID NO: 689)
VSNEIKIIALKLSIFWGHNHFRLTGNWKIFYLCLKSGLA
>orf00552
(SEQ ID NO: 690)
MKRIQLNMNETKKYLVIKAIAQGKKTKKRACVELNLSERQINRLLLAYQQKGKEAFRHGH
GNRNRKPKHAIPDEIKERVLKKYLSYETYKPNVLHFCELLAEEEGIKLSDTTVRKILYKK
NILSPKSHRKTKKRVRKQAKLNLNQPLDNPILPTAKDFLEDPKKVHPSRPRKKFAGELIQ
MDASPHAWFGPETTNLHLAIDDASGNILGAYFDKQETLNAYYHVLEQILANHGIPLQMKT
DKRTVFTYQASNSKKMEDDTYTQFGYACHQLGILLETTSIPQAKGRVERLNQTLQSRLPI
ELERNNIHTLEDANTFLLSYIQTFNEQFGNKTKLSVFEEAPNPSERNLILARLAERVVDS
GHHIRFQNRYYMPVEQGKEVYFIRKTKALVLKAFDGDIYLNIADKIYHTKELLDHELYSK
NFEQEPEQKKKDASISLHKPIRGNSHLSNNTFIKIKRIMKSLLVRSFILLNYKYNLFFAK
WEAFP
>orf00556
(SEQ ID NO: 691)
LVEQLTFNQWVTGSSPVRVIYAGLAELADAPDLGSGA
>orf00557
(SEQ ID NO: 692)
MGEEEMRNKMIIAVSLVVAGVMTYLMFSGLDEDFYHFP
>orf00567
(SEQ ID NO: 693)
MNTIERTRRLVKGCATHCFEVVDRTDEVSSKHVFEVVDETNEVSSKHVFEVVDETDEVSN
HTYGKAT
>orf00581
(SEQ ID NO: 694)
MLSNDFIQLRKDDIKTTSVLYFPIRLFSLETMNMSSQYF
>orf00582
(SEQ ID NO: 695)
LTCYPNPQKRLEAGFDKLIEIKRLTASKIQDILSVAPRSIGTTSPAREFEIIENIKHYKR
LIDKAKKCVNDLMAEFNSVITTVTGIENRLGAVILAEIRNIHAFDNPAQLQAFAGLDSSI
YQSGQIDLAGRMVKRGSPHLR
>orf00595
(SEQ ID NO: 696)
MKRIQLNMNETKKYLVIKAIAQGKKTKKRACVELNLSERQINRLLLAYQQKGKEAFRHGN
RNRKPKHAIPDEIKERILKKYLSYETYKPNVLHFCELLAEEEGIKLSDTTVRKILYKKNI
LSPKSHRKTKKRVRKQAKLNLNQPLDNPILPTAKDFLEDPKKVHPSRPRKKFSGELIQMD
ASPHAWFGPETTNLHLAIDDASGNILGAYFDKQETLNAYYHVLEQILANHGIPLQMKTDK
RTVFTYQASNSKKMEDDTYTQFGYACHQLGILLETTSIPQAKGRVERLNQTLQSRLPIEL
ERNNIHTLEDANTFLLSYIQTFNEQFGNKTKLSVFEEAPNPSERNLILARLAERVVDSGH
HIRFQNRYYMPVEQGKEVYFIRKTKALVLKAFDGDIYLNIADKIYHTKELLDHELYSKNF
EQEPEQKKERRKYIPPQTHPWKLTSFKQYLHKNKKDYEEFTSEEIHSPQLQV
>orf00601
(SEQ ID NO: 697)
MDTKSSCLITTGRNDSPSTCLPRVASNNDRFSSEFRIIPDFHCSKKGIHVNMDDFS
>orf00604
(SEQ ID NO: 698)
MMSIREQDLKDIGAIIKYKNFHSPFDTFKYLKDMGFDTIDLSVLLEGFSYAYGMDWLEKF
FKENQDKLREFY
>orf00610
(SEQ ID NO: 699)
MIPLYRTDNDITKFFTKIRNGHLAKTAGGLDDKFHEANASTSKAFDRQGVGEVNDIRDSA
GSQELRINDKRKTENILFLEIRVRIFRVPHPNDSFFSSHFLG
>orf00611
(SEQ ID NO: 700)
VLSQGDKDITILDAGLLKNGKIGPVTKDTNDIKATDNMIENSFVLLNQQNIMLFCNQGAT
EGKTNFSPSDKDNFHNKTYFFMM
>orf00616
(SEQ ID NO: 701)
MKIKEQTRKLAAGCSKQCFEIVDRTDEVSSKHGFEVVDETDEVSNHTYGKAKLTWFEEIF
EEYKMMGKAGQLVFFDVYRLVRQVS
>orf00645
(SEQ ID NO: 702)
LVEIVRGGSPRPIKDYLTSEVDGINWIKIGDTEKGEKYINNVKEKIKKSGLNKTRFVKKG
TFLLTNSMSFGRPYILNVDGAIHDGWLAISNYENSLNKDYLFYILSSNVVYSQFLSLISG
AVVKNLNSDKVASILIPLPPLAEQQRIIEAIESALEKVDEYAESYNRLEQLDKEFPDKLK
KSILQYAMQGKLVEQDPNDESVEVLLEKIRAEKQKLFEEGKIKKKDLDISIVSQGDDNSY
YEEVPCEIPESWEWVRLNDITSYIQRGKSPKYSNIPIYPVIAQKCNQWSGFSIDLARFID
PETVHSYQKERLLRDGDLMWNSTGLGTLGRLAIYHENKNPYVWAVADSHVTVIRVLSGVI
NCHFIYNFLSSPIVQSVIEEKASGSTKQKELLTKTIKEYLIPLPPLPEQSRIVDKIEQFF
AHIDALI
>orf00657
(SEQ ID NO: 703)
MTPEQLKASILQRAMEGKLVPQNPNDEPASELLKRIKAEKEKLISEGKIKRDKKETEIFR
GDDGKHYGKFADGSTQEIDVPYDIPDTWEWVRFSTLVEIVRGGSPRPIKDYLTSEVDGIN
WIKIGDTEKGEKYINNVKEKIKKSGLNKTRFVKKGTFLLTNSMSFGRPYILNVDGAIHDG
WLAISNYENSLNKDYLFYILSSNVVYSQFLSLISGAVVKNLNSDKVASILIPLPPLAEQQ
RIIEAIESALEKVDEYAESYNRLEQLDKEFPDKLKKSILQYAMQGKLVEQDPNDESVEVL
LEKIRAEKQKLFEEGKIKKKDLDISIVSQGDDNSYYGNIPMNWVVIKIKDIFSINTGLSY
KKGDLSINKGVRIIRGGNIKPLEFSLLDNDYYIDTQFISSEQVYLKHNQLITPVSTSIEH
IGKFARIDKDYDGVVAGGFIFQLTPFESSEIISKFLLFNLSSPLFYKQLKAITKLSGQAL
YNIPKTTLSELLIPLAPFEEQELITQKVEKLFEKVNQLWK
>orf00669
(SEQ ID NO: 704)
MCKANSRNDIFILQDSFCFEIFSRKKFKIVKEVLPNSTCKFRVVQ
>orf00673
(SEQ ID NO: 705)
VDRTDEVSSKHCFEVVDRTDEVSNHTHDKPTLTWFEEIFEEYHSPFHN
>orf00674
(SEQ ID NO: 706)
LDNIHIVLDSLNAVSGIQDFICDGLAIFCNQITSGCSSCK
>orf00683
(SEQ ID NO: 707)
MGPLLMHLCQQLVWLAKYLKRAGSDMMFLQEFLNRRFNPSLLGKIIL
>orf00684
(SEQ ID NO: 708)
LVAKGQGHKLRVSRHKDNQGIGVLFPNLSSHFQPLHLLISNLNIQKE
>orf00692
(SEQ ID NO: 709)
MTSYHRTFVPQIDARDCGVAALASIAKFYGSDFSLAHLRELAKTNHEGTTALGIVKAADE
MGFETRPVQADKTLFDMSDVPYPFIVHVNKEGKLQHYYVVYQTKKDYLIIGDPDPSVKIT
KMSKERFFSEWTGVAIFLAPKPSYQPHKDKKNGLLSFLPLIFKQKSLIAYIVLSSLLVTI
INIGGSYYLQGILDEYIPNQMKSTLGIISVGLVITYILQQVMSFSRDYLLTVLSQRLSID
VILSYIRHIFELPMSFFATRRTGEIISRFTDANSIIDALASTILSLFLDVSILILVGGVL
LAQNPNLFLLSLLSIPIYMFIIFSFMKPFEKMNHDVMQSNSMVSSAIIEDINGIETIKSL
TSEENRYQNIDSEFVDYLEKSFKLSKYSILQTSLKQGTKLVLNILILWFGAQLVMSSKIS
IGQLITFNTLFSYFTTPMENIINLQTKLQSAKVANNRLNEVYLVESEFQAPENPVHSHFL
MGDIEFDDLSYKYGFGRDTLTDINLTIKQGDKVSLVGVSGSGKTTLAKMIVNFFEPYKGH
ISINHQDIKNIDKKVLRRHINYLPQQAYIFNGSILENLTLGGNHMISQEDILKACELAEI
RQDIERMPMGYQTQLSDGAGLSGGQKQRIALARALLTKAPVLILDEATSGLDVLTEKKVI
DNLISLTDKTILFVAHRLSIAERTNRVIVLDQGKIIEVGSHQELMQAQGFYHHLFNK
>orf00699
(SEQ ID NO: 710)
LRIYLHEPLITTVSQDFSSLSDISATHFEQLHIVAIVHSDIQRNNSPLTCDNRLSLHSVK
FLFTRIIG
>orf00723
(SEQ ID NO: 711)
MGLIKTLAKIYGNYFLTVQGVKVMKTIKKDDNAVVGLGKLFIADKLMDTARWLIKPEDKK
>orf00724
(SEQ ID NO: 712)
MKFFWGLLAILFIKPIIGIVKFFWMIISFAVQLLFYKILDWFFKLI
>orf00725
(SEQ ID NO: 713)
MKIKEQTRKLAADCSKQCFEVVDRTDEVSSKHRFEVVDRTDEVSNHTYSKVKLTWFEEIF
EEYKMILLLILYHMERD
>orf00733
(SEQ ID NO: 714)
MHSQTFQFLLMTDKTSLLHRKHRSFIRNIHSKFLILFDLLCGILSRNDSNHNPIS
>orf00736
(SEQ ID NO: 715)
MARTELPDKIETERLVLRVRTVADAEDIFDYASLPEVAYPAGFPPVKTLEDEIYYLEYIF
PERNQKENLPAGYGIVVKGTDKIVGSVDFNHRHEDDVLEIGYTLHPDYWGRGYVPEAARA
LIDLAFKDLGLHKIELTCFGYNLQSKRVAEKLDFTLEARIRDRKDAQGNCCDDLRYALLK
SEWEVI
>orf00741
(SEQ ID NO: 716)
MGKIVAIDLFNGAGGTTSGLKKSGIDVQVAVEIDSVAVKTYKLNNPEVSVIDME
>orf00746
(SEQ ID NO: 717)
LLRKQEGEYLRAENAILKKLRELRLKEEKEKEERQKLFKN
>orf00768
(SEQ ID NO: 718)
LKHLFCHFNPLWIDEIIRLAYKDQDTKDVKSKVKIGN
>orf00792
(SEQ ID NO: 719)
LCCNRHIANLDLEFISYYLGQVGFDTRISTGLGIFVTKIGNVLFDTDNQFASFLNVCDTS
ISLDWFGSSKAEKANQ
>orf00817
(SEQ ID NO: 720)
MKTKEQTRKLASGCSKHCFEVVDGTDVVSSKHCFEVVDRTDEVSNHTHGKATLTWFEEIF
EEY
>orf00819
(SEQ ID NO: 721)
MDFFFMNEVKEQVLFRDNHSEHIFWIEGVSDFMIKVNTALW
>orf00839
(SEQ ID NO: 722)
MEELVTLDCLFIDGTKIEANANKYSFVWKKTTEKFSAKLQEQIQVYFQEEITPLLIKYAM
FDKKQKRGYKQSAKNLANWHYNDKEDSYIHPDGWCYRFHHIKYQKTQTDFQQEIKVYYAD
EPESAPQKGLYMNERYQNLKAKECQALLSPQDRQIFAQRKIDVEPVFGQIKACLGYKRCN
LRGKRQVRIDMGLVLMANNLLKHSEMK
>orf00840
(SEQ ID NO: 723)
MHIHYNTNQTTLPLEISSFLPQDHLVFTIEKVVNTLEERHFYAFYHAFGRPSYHPKMLVS
TLLFAYSQGIFSGRKIEKWKS
>orf00843
(SEQ ID NO: 724)
LRLWVIFVIMKVIKSYNTLNDYYRKLFGEKTFKVPIDAGFDCPNRDGTVAHGGCTFCTVS
GSGDTIVAPDPPIREQFYKEIDFMHRKWPDVQKYLVYFQNFTNTHEKVEVIRERYEQAIN
EPGVVGINIGTRPDCLPDETIEYLAELSECMHVTFELGLQTTYEATSDLINRAHSYEL
>orf00845
(SEQ ID NO: 725)
VETVKRLRKYPKIEIVSHLINGLPGETHEMMVENVRRCVTDNDIQGIKLHLLHLMTNTRM
QRDYHEGRLQLMSQDEYVRVICDQLEIIPKHIVIHRITGDAPRDMLLGPMWSLKKWEVLN
SIEMEMRRRGSVQG
>orf00853
(SEQ ID NO: 726)
MKIKEQTRKLAAGCSKHCFEVVDKTDEVSHIHTVRRR
>orf00859
(SEQ ID NO: 727)
VQVCVFTNFCFFHCFSSLANCRLFNLRGICLPCISYQ
>orf00868
(SEQ ID NO: 728)
VFKKDRFSIRKIKGVVGSVFLGSLLMAPSVVDAATYHYVNKEIISQEAKDLIQTGKPDRN
EVVYGLVYQKDQLPQTGTEASVLTAFGLLTVGSLLLIYKRKKIASVFLVGAMGLVVLPSA
EAVDPVATLALASREGVVEMDGYRYVGYLSGDILKTLGLDTVLEETSAKPGEVTVVEVET
PQSTTNQEQARTENQVVETEEAPKEEAPKTEESPKEEPKSEVKPTDDTLPKVEEGKEDSA
EPAPVEEVGGEVESKSEEKVAVKPESQPSDKPAEESKVEQAGEPVAPREDEKAPVEPEKQ
PEAPEEEKAVEETPKQEDTQPEVVETKDEAANQPVEEPKVETPAVEKQTEPTEEPKVEQV
GEPVEPREDEKAPVSPEKQPEAPEEEKTAEETPKQEDKIKGIGTKEPVDKSELNNQIDKA
SSVSPTDYSTASYNALGPVLETAKGVYASEPVKQPEVNSETKAEKVAANTDAKQSEVNSE
TASLKTAISGLNTDKVELENQLKIAQGKTETDFSMESWTVLSTAKNKAQEVKDNGTATQE
QINEAEKSLKTALADLSVDKTALGSAIDTATKKNKENYTNQTWAELETVLTAAKSVNTNE
SKQSEVNEAVEKLTATIEKLVELSEKPRLTLSIEKRDIDRKVTVTYTLENPANTQIKSIT
ATLKKGEEVVKDFVLTEENLKTNHLTALFEKLDYYKEYTLSTDMVYNRGNDDETESISEE
LIQLNLKKLELKDIQTVSLMKFENGQESQVTHLSDKPTDLSKLYLKVTSSTSKDAVLAVS
SIEEEIVENKKIFKIHADTPELVVRKKDGSLSKGFDYYMERVIPHDGDIYYDFKDLISAM
TSNPTGTFILGRDISSRNVKPDGNGKSYIKGEFKGKLLGTNDNVRHSIFDLEYPLFDTIK
SGVVKDIDFKHVNMVFPDSNQGDNVATIARVIKDKTKIENVNVEGYLEGRDHVAGLVNNL
EGNSEIENISFTGKIKSKGGNSITAGIAGRNILSRVKRAYVNANIEVLGSTNSSMLVAVN
GTTLNASGGWGAWGRLTESVAKGTLEIKRSGQAGGVTATVWPYGAIDKVVSYAKVTKGKE
LFGSDGDLNNNWFMQKINNIFGVQGISSGDSGNDSKFKRISEEEAKQKVASYNITAPNLM
SDSSLLVDRLNESWKNTDQFESIQDYQSQNQLIYQNLTKFTPYYNKEFIVHEGNALTPEQ
EILKTKKIKSIVGLKGTEFVVDGSDIDTIMLHFEDGSQKRYKVTSTGKFSITNLPEYQVE
DLNVVYTSEHIVHPLDSSLINNLVEELKKVELYTESTYQVLGIDKDNANKLNRTKRLFLD
ESLDAVKTQLPTFVKTMFENEWLHINGESSGAVAALRQKIMDNKTAILLALTYINRYYDV
KFSDYNIKKLMLFKPTFHGEKIDLLDRLIRLGSSGENRLKGSENAETFKQLFASETKQKD
LVTYLDYNRSLLTNYQTTGEWFKETTKDYIQFEERPSLVEEIKDAKYRVYDNLTAPYYQG
YILPLLTLKNTHLAILSNYSTMTFVSREKRPNWKNEDFDKWVKYVATAHRNHVDTWYKIL
PDNIKGKMVKENVTAVWEGLSIPGSEWVDQNAVDRKGRDYAPAREFFNLVGGPMGGWYAY
HGYGAHAGGRNRVNYEVFDVLSEYGISVFTHELTHVNDTWIYLGGYGRRENMGPEAYAQG
LFQSPVPGQPGWGALGLNMAFERKNDGDLIYNASPTQFENRKELDSYMKNYNDTLMMVDY
LEGDAVISKGKEAITKWFKKVEPKVVSQTAQYDTVRQLTAEEKEKLSVSSVDDLVDQGLM
SDRAVGNNTYNPADFETSYIAIDYMTGIYGGGKNSVGSPGALMFKHNTFRMWGYYGFEEG
VLGYASNKFRQASRDEGHAGLSDNFIISKISKGEFLTMEAFKKGYFKKVVEELKTKGIRP
VTINQKTYSTFEELQEGFKQAVERDLKKNQLDERETRNFKFQVFRQLLQQTDSFKTSIFR
>orf00883
(SEQ ID NO: 729)
VGNRIFIAFLQKLGLLDNLTGIREKLHPITGQGNTLGIADKDFNAHFIFQISHCIGETGL
SDKELLGCLIHRASFDDFDNIM
>orf00892
(SEQ ID NO: 730)
MKTKKHRLLALALISSFTLLGAASAAVQYPDGGVWTYGEGSGGGWAFSNYYHGKKYHYSS
LVSRWNSHSDKGEASAGKTSYAWIWTKWGEQVAFYCDYD
>orf00903
(SEQ ID NO: 731)
MSMIEVSHLSKSFGDKIALNDISFTVKEGQIFGFLGPSGSGKTTTINILTGQLLADKGQS
IILGQKSQNLTSGELKRIGLVSDTSGFYEKMSLYNNLLFYSKFYNISKLRVDNLLKRVGL
YDSCKMVAGKLSTGMRQRMLLARALINKPAVLFLDEPTSGLDPTTSRTIHELILELKTAG
TTIFLTTHDMNEATLLCDYVALLNKGKLVEQGAPSELIQRYNKDKKIKVTDYNGNQITFD
FTSLEQVSQADLENIFSIHSCEPTLEDIFITLTGGKLNA
>orf00911
(SEQ ID NO: 732)
LISNKVDITLANFTVTDERKKQVDFALPYMKVSLGVVSPKTGLITDVKQLEGKTLIVTKG
TTAETYFEKNHPEIKLQKYDQYSDSYQALLDGRGDAFSTDNTEVLAWALENKGFEVGITS
LGDPDTIAAAVQKGNQELLDFINKDIEKLGKENFFHKAYEKTLHPTYGDAAKADDLVVEG
GH
>orf00912
(SEQ ID NO: 733)
MKLFKPLLTVLALAFALIFITACSSGGNAGSSSGKTTAKARTIDEIKKSGELRIAVFGDK
KPFGYVDNDGSYQGYATILN
>orf00946
(SEQ ID NO: 734)
MTGKKGFLFLNCHICMVTTTTCFLKERVESELLIFFTFC
>orf00948
(SEQ ID NO: 735)
MDTPDENGYVADDYRITYLEAHIKAMRDAIYQDGVDLLGYTTWSCIDPVSAGTGEMNKRY
GFIYVDRDNVGNGTLKRSKKKSFYWYMSFIAMV
>orf00953
(SEQ ID NO: 736)
LSCQIAFCLIDRLDYPIMFSKVCQENHFQVFTPFSKKLKNFLKNA
>orf00966
(SEQ ID NO: 737)
MFLGMIGNISIILQFFGITIIVKIDNQARAIDFFKHDKSSF
>orf00968
(SEQ ID NO: 738)
MFSLNFFDDNVFLSIKIAHKGCFQLLDMTNPNFFNKFFLAQASDQLLHFLSWNIEL
>orf00978
(SEQ ID NO: 739)
MTEPDFWNDNIAAQKMSQELNELKNTYNTFHKMEELQDEVEILLDFLAEDESVHDELVAQ
LAELDKIMTSYEMTLLLSEPYDHNNAILEIHPGSGGTEAQDWGDMLLRMYTRYGNAKGFK
VEVLDYQAGDEAGIKSVTLSFEGPNAYGLLKSEMGVHRLVRISPFDSAKRRHTSFTSVEV
MPELDDTIEVEIREDDIKMDTFRSGGAGGQNVNKVSTGVRLTYIPTGIVVQSTVDRTQYG
NRDRAMKMLQAKLYQMEQEKKAAEVDSLKGEKKEITWGSQIRSYVFTPYTMVKDHRTSFE
VAQVDKVMDGDLDGFIDAYLKWRIS
>orf01011
(SEQ ID NO: 740)
MQVIKRNGEIAEFNPDKIYQAILKAAQTVYVLTDDLRQNLAQVTKKVVLDLQEAKVERAT
ISMIQSMVEHRLLGAGYITIAEHYISYRLQRDLERSGYGDHIAVHLHFEQIR
>orf01015
(SEQ ID NO: 741)
MKIKEQTRKLAAGCSKHCFEVVDETDEVSSKHCFEVVDETDEVSNHTYGKAKLMRFEEIF
EEY
>orf01068
(SEQ ID NO: 742)
MKVINQTLLEKVIIERSRSSHKGDYGRLLLLGGTYPYGGAIIMAALAAVKSGAGLVTVGT
DRENIPALHSHLPEPMAFSLQDQQLLKEQLEKAEVVLLGPGLRDDASGENLVKQVFVNLS
QNQILIVDGGALTILARTSLSFPSSQLILAPHQKEWEKLSGITIEKQKEDATASVLTSFP
QGTILVEKGPATRIWEVGQSDYYQLQVGGPYQATGGMGDTLAGMIAGFVGQFRQASLYER
VAVATHLHSAIAQELSQENYVVLPTEISRYLPKIMKIICQQERGSKDKLV
>orf01077
(SEQ ID NO: 743)
VLDSKEELKESENDAPKLETPLREEPRLAPQTLPEASEVLENKREESKVEIIEPAQADDI
RKVVGELAKDISITKLYMTGHSLGCYLAQIAAVEAYQKYPDFYNHVLRKVTTFSAPKVIT
SRTVWDAKNGF
>orf01091
(SEQ ID NO: 744)
LSYSILICLCNSTINESLRAFYCWQKFITFNQVTGNARGKGTTCTSIGPDN
>orf01094
(SEQ ID NO: 745)
MGRKPRTRPEERTELERLQAENEYLRAENAILKKLRELRLKEEKEKEERQKLFKN
>orf01096
(SEQ ID NO: 746)
LSTCWNGKFCHICVALFHCFRAFKLALNEILCLLTNVSFIFVSVAF
>orf01109
(SEQ ID NO: 747)
VVLSTSAILVACGKTDKEADAPTTFSYVYAVDPASLGYSIATRTSRTDVIGNVIDGLMEN
DKYGNVAPSQKDYDLNSTGWAPSYQDPASYLNIMDPKSGSAMKHLGITKGKDKDVVAKPG
LDKYKKLLEDAVSEITDLEKRYEKYAKAQAWSTDSSLLMPTASSGGFPVVSNVVPFSKPY
SQVGIKGEPYIFKGMKLQKDIVTTKEYNEVFKKWQKEKLESNSKYQKELEKYIK
>orf01113
(SEQ ID NO: 748)
LNFDFFIFLAHFIPLFTFSILQENPKTSKKKLYIRLL
>orf01119
(SEQ ID NO: 749)
MGFSMKLIHDLNTHTTHSTAKMLYNVKAIKNDFSIRE
>orf01134
(SEQ ID NO: 750)
LIRIIRNIYRSGEGNTSVFQSFIDQINSNQFCYGSNFDRLRCILLIENFTSICLNSNRMF
SGNGKILSNSSRSTP
>orf01137
(SEQ ID NO: 751)
MNATDIKNTYLKYIKENAVFNDVTDTHTEVITPFIDPLGEAIGFSIKSNGKHLTVTDDGY
TIWNLSINNIDVTKKGRRQDIFNSLLHFNGFDLHDGAIERTTGKEHLGQVIHDMTQLLMN
VYDFIQLTPNNIKSQFLDDVKSYFMKNEHYTVFPAFSIAGKSRLEHRFNFVFMSKGISKI
ARVHNNITKQQVDTILASWLDTSEYRRKEYGDTEQLYIIVSDEGYNNIKDDHQIALQEYG
INILNFSDKEQLEIQLGK
>orf01138
(SEQ ID NO: 752)
MSKVVKVTGAEVVISHNEEYLKVNPSELNFVPKLGDEVEVHKVDGEIIVIKVKDKKDDKI
NINIVNENNAMQNQSQVVHTQEIATGVHYVNKWVYVILALFLGGLGIHHFYAGYNGKGFL
FLILSLTGIPAIIALFQGIIALFKKPDVYGRIAV
>orf01149
(SEQ ID NO: 753)
MKIKEQTRKLAAGCSKHCFEVVDRTDEVSSKHGFEVVDETDEVSSKHGFEVVDETDEVSN
RTTVRRR
>orf01156
(SEQ ID NO: 754)
LQNDKNHKLFDNYTCQKEKDVLQCKQVKRKEERSYDVGTRIYTIYYFLLF
>orf01158
(SEQ ID NO: 755)
VDRTDEVSSKHGFEVVDETDEVSNHTYGKVKLTWFEEIFEEY
>orf01159
(SEQ ID NO: 756)
LFFKDEKQALYTKPKTKSSSFRASKVSNQTIVATTRTDCQVIALNLCDKLENGVVVVVQT
THHIGIDDVIYSKIFQHLTHSIEMSLAFFIKKVQDRRRILYCHLVFFFLRVQDTKRIFLQ
ATLAILRQGLLERCQIVNQGLAVGCTALRISKSVEVQFDTLNTDFLQKMGCHSDCFHIGS
WIARAKTLNTNLVELAQAPCLWTLITEHRSHIVELAWLLHFWGEEFIFHIGTDNGRSSFW
TEGNMTVTLVIKIVHFLGYDIRCISDRATDNLVMLKNRRAHFCIVIALENLTGKALNVLP
LSRFSR
>orf01164
(SEQ ID NO: 757)
MEQIGKVFRQLRESRNISLRQATGGQFSPSMLSRFETGQSELSVEKFLFALENISASVEE
ILFLARGFQYDTDSELRKEITDVLEPKNIAPLEDLYRREYQKHAHSHNKQKHILNAIMIK
SYMKSIDERVELTAEEGKVLHDYLFSTEIWGIYELNLFSVSSPFLSVSLFTRYVREMVRK
SDFLMEMSGNRNLFHTILLNGFLASIECEEFTNAYYFKRVIEEHFYKENETYFRIVYLWA
EGLLDSKQGRVKEGQKKMEDAVCIFEMLGCNKSAEYYRNTTEC
>orf01165
(SEQ ID NO: 758)
LIPYFLHFIIFFRKFIKNLPNCQNYEKIEDIYHVEGLL
>orf01167
(SEQ ID NO: 759)
MKIKGQTRKLAAGCSKHCFEVMDRTDEVSSKYCFEVVDRTDEVSNHTYGKATLT
>orf01170
(SEQ ID NO: 760)
VVPFSDTFKDRNQVDIFTIKISRCNSSTIGENSWDIHISNSNHRSRHVLVTATDSDEGIH
VVTTHSRLDGVRDDVTRC
>orf01190
(SEQ ID NO: 761)
MKKVKLGEVLSLKKGKKATVLAEQTTLSQRYIQIDDLRNNNNLKFTESLNMTEALPDDIL
IAWDGANAGTVGYGLSGAVGSTITVLKKNERYKEKIISDYLGVFLESKSQYLRDHSTGAT
IPHLNKNILLDLQLELLGIEEQENIICILNTIKRLITKRKFQLDELNLLVKSRFNEMFEE
YPDSVFLDTYIKELRAGKSLAGEENNKNKVLKTGAVSYDYFNSSEVKNLPIDYIPLDEHK
VEIGDVIISRMNTSELVGAAGYVWAINSDNIYLPDRLWKVILNDRVNPVFLWKLITNEKT
KLKIKRISSGTSGSMKNISKSQLLQIRVPFPPLALQNEFADFVALVDKSQLAIQKSLEEL
ETLKKSLMQEYFG
>orf01199
(SEQ ID NO: 762)
MKIKEQTRKLAAGCSKHCFEVVDKTDEVSSKHGFEVVDETDEVSNHTYGKATLTRIEEIF
EEYKSS
>orf01226
(SEQ ID NO: 763)
MRLSIQLIHDLNTHTTHSTAKMLYNVKAIKNDFSIRE
>orf01237
(SEQ ID NO: 764)
LVYAPFSFNILLDYITFDFKILLFSVFLAINRFHNDFIQFLL
>orf01240
(SEQ ID NO: 765)
LGTKIGISKNTIGNYEKRVKSTKKNTIFDLAKVFSSLIDALFPPVQKDSPSDIQSIYDQR
APPRQGKVLTYA
>orf01246
(SEQ ID NO: 766)
LFVFILIFLKSSIYIGIFWFIDFGKAVDFQGWKVLFEFFMVVIDQFSFGCNPGVVFILPG
IALGQQSIDTRICDTVDNAESEQKLTIGMTGVVIDKACKLDCLALKFIWIVVDSLHDFHI
VFISDLNTILG
>orf01247
(SEQ ID NO: 777)
MTVGFDLLHPDIQLNNCQDKGKHHGDIGQIGIVHVDVLV
>orf01249
(SEQ ID NO: 778)
MEGVAKGRIGRKKNNGIDNRCCHKKRNGRVTWNLFFQKTIDDGDDSTFTRREKYTDKGPK
KDSPPTISREKMINLVRCDINFNQP
>orf01262
(SEQ ID NO: 779)
VVIGVASATTNIWIIFLSGFTAILAGAFSMAGGEYVSVSTPKDTEEAAVSREKLLLDQDR
ELAKKSLYAAYIQNGEFKTSAQLLTNKIFLKNPLKALVEEKYGIEYEEFTNPWHAAISSF
VAFFLRSLPPMLSVTIFPSDYRIPATVLIVGVALLLTGYTSARLGKAPTKTAMIRNLAIG
LLTMGVTFLLGQLFSI
>orf01277
(SEQ ID NO: 780)
MILMTKNINLTNEELELIQGGADPYGKEPNGYYPWKMEPVLTLLVHGFCPRDTDDLGYIG
GGNHLCKGSAARF
>orf01282
(SEQ ID NO: 781)
LQVGQANEIGDPGAHFTQWNLLDDIGFDQLIQPNQKQYNDCHYCSFFHDFLF
>orf01301
(SEQ ID NO: 782)
LLHICIGETFDCIPYCMLTFFLSKSIGLTILLHKVKTVVFIDDQSNDKTCKICIHISFFR
IKLSQQCQLSFSVYF
>orf01309
(SEQ ID NO: 783)
MTTIFLTRTSCSNCGKQSTFERFDRVYAAKTPEIISAILDWDFFKFTCHNCNHKVLIDYP
TVVVDEEQKTIIQYCADGNVDVLSMQICSLISEGVNLSEYRIRVVSDIESFVEKVQIVSV
GYDDRAIELMKYMNSPLEDGDIQFNYEHMVETKVGHENYQFMFINNQIAVASLDFSQEQY
EYYLADVEDLETNTYYIDSRWAESFFRTSLA
>orf01310
(SEQ ID NO: 784)
MVRRNSKITRQQKKIRDAFVSERTVEIIPAKREFTDVKTKKLRVAAYCRVSTFDESQSGS
FELQKQTYTERINSNPDWIMAGIYADQGASGTSIKRREQFQQMLHDCRCGKIDLIIVKSV
SRFARNQLDFISIYRELKALSPPVGIYIEDINLNTLDTNSEFILGIMAIVAQGESEQKSA
SITWSVIERFKRGIPMIPTHNLLGYTKDQYGRVVIDETEAKIVRLIYDSYIEGMTASEIA
STLMTNHIPTVTGLERWTSLAVYNILRNEKYKGEIIMQKTYTVDCFSHKTRKNNGEKPKY
RLKNGIPSIIPESRWDLVQELLKQPRRKSKSTSEIFVPKLYIKKLKSGKLRDFVVLDPSW
KSEDIHEVFK
>orf01311
(SEQ ID NO: 785)
MTVNKNEVTFSKGIVQALEYPAHVLVAFNKDTKVMGIQVCRAKTRGAFSFSKPVGEQKGI
VQVGHKTLKETLLTIMSEWKSDKRYRVEGIHIPEDKAFVFELKDFDELSDERKNDNR
>orf01313
(SEQ ID NO: 786)
MEKYNNWKLKFYTIWAGQAVSLITSAILQMAIIFYLTEKTGSAMVLSMASLLGFLPYAVF
GPAIGVLVDRHDRKKIMIGADLIIAAAGSVLTIVAFYMELPVWMVMIVLFIRSIGTAFHT
PALNAVTPLLVPEEQLTKCAGYSQSLQSISYIVSPAVAALLYSVWELNAIIAIDVLGAVI
ASITVAIVRIPKLGDRVQSLDPNFIREMQEGMAVLRQNKGLFALLLVGTLYMFVYMPINA
LFPLISMDYFNGTPVHISITEISFASGMLIGGLLLGLFGNYQKRILLITASIFMMGISLT
ISGLLPQSGFFIFVVCCAIMGLSVPFYSGVQTALFQEKIKPEYLGRVFSLTGSIMSLAMP
IGLILSALFADRIGVNHWFLLSGTLIICIAIVCPMINEIRKLDLK
>orf01315
(SEQ ID NO: 787)
MELILKAKDISVEFKGHDVLDINELEVYDYDRIGLVGANGAGKSTLFKVLLGELIPPGCK
MNHLGELAYIPQLDEVTLQEEKDFALVGKLGVEQLNIQTMSGGEETRLKIAQALSAQVHG
ILADEPTSHLDREGIDFLIGQLKYFTGALLVISHDRYFLDEIVDKIWELKDGKITEYWGN
YSDYLRQKEEERKRQAAEYEQFIAERARLERAAEEKRKQARKIEQKAKGSSKKKSTEGGG
RLAHQKSIGSKEKKMHNAAKSLENRIAALGKVEAPEGIRRIRFRQSKALELHNPYPIVGA
EINKVFGDKALFENASFQIPLGAKVALTGGNGTGKTTLIQMILNHEEGISISPKAKIGYF
AQNGYKYNSNQNVMEFMQKDCDYNISEIRSVLASMGFKQNDIGKSLSVLSGGEIIKLLLA
KMLMGRYNILIMDEPSNFLDIPSLEALEILMKEYTGTIVFITHDKRLLENVADVVYEIRD
KKIKLKH
>orf01316
(SEQ ID NO: 788)
MNQLEFQRNHLQMDYYSESYQDFERDFYRYSNMNIPLTFLTDDILKTMATSRKNYFVLNK
EKSRDNRDHFFIFEVRTLEENPLIYHYTYKKTTTYLAEK
>orf01317
(SEQ ID NO: 789)
MQKWMGEFLSEHTSALTDDANKVTYMSDLSLEKKLLLLSQVYAGQLNTRIHVVKKNNQVS
YTGTIPSLTKDFILIKTTTGHINLKLKDIVSIELVEEVLYESA
>orf01318
(SEQ ID NO: 790)
MKKSINAQKKIDPANLPKTMVGHVLELFRKKYTSGAVRQIGVSYGGFVDENFTLLSLFDD
VEQIEKENRLQTAIDVVREQFGFLAIQKGTVLTEGSRNIERSKLIGGHSAGGLEGLK
>orf01343
(SEQ ID NO: 791)
MNXXFISTKDKHTLIQVSAVRFRDGREIDAYDSYVHTSVPLKSFINEFDRGLQLRP
>orf01363
(SEQ ID NO: 792)
MIAMRSYITLTCNLNNNLFCLNSFFLTNLVWSQIFSLLSVFITVYI
>orf01364
(SEQ ID NO: 793)
MVFDANRIISEDSEGFVIPHGDHNHYIKVQTKGYEAALKNKIPSLQSNYQPGTFDEKAVL
AKVDQLLADSRSIYKDRLS
>orf01390
(SEQ ID NO: 794)
MARLEPAKIAKIVLGILLYIIDLIKSSFVLPIPKAAKKSLILISFVPSFNDKNIVIRRPR
QITKIMPRFICFLFRIFACIS
>orf01396
(SEQ ID NO: 795)
MASKRLSIEEQIEKKEESIKQLQNQKRQLKKKLNEQERKARNKRLIEKGAVFESIFEESI
DLTKDEFYKLIKTLNDEEIRLNIMEILEERIDDNVEKSSKDEIT
>orf01397
(SEQ ID NO: 796)
MADSFHFSVNIISRGKGKSAVASAAYISGEKIKNEWDGVTHDYTRKEKILVKNIILPDHI
PKEFNDRSTLWNKVEMAEKNSNAQLARQFIIGLPKELSLSENKNLVERYIKENLTSQGMI
VDYAIHDESQDKNGNIHCHIMTIMRPINEKGEFLAKSKKEYILDEKGEKVLNKNGKPKTR
KVELTTWNDTGNVEKWRENFSDLCNKYLERAGAEKRVDHRVLKDKIQIIYRQSI
>orf01398
(SEQ ID NO: 797)
MERKGIETDKGNYNREIRKYNQLVKTIKEEIKTLKGWIGNLLDNLSTAYEKFKDIERDKV
IDNPKLFNLTNYLLTYSEIQKEKSKYLKGYAKTNKEKYDFKKLTSAYSYLRKNNIETIGQ
LQTKIETLKSNSYRLNKKAKTIHKEMEDVEKKILYYEIYKAKKEVYEEYQKKNIFTKEAF
YNKHKKDIDQYKVVSGKLKKLLSDKEKLSPKKWNEEKILLMSNLEEINKEKDKIKDEYQE
INHIKYSVDFVNKELGIDLSIEIDKLIKQGEKPSVIAQIKKFQDQVNKDNEYREMMKNKK
MDQER
>orf01407
(SEQ ID NO: 798)
MELSAIYHRPESEYAYLYKDKKLHIRIRTKKGDIESINLHYGDPFIFMEEFYQDTKEMVK
ITSGTLFDHWQVEVSVDFARIQYLFELRDTEGQNILYGDKGCVENSLENLHAIGNGFKLP
YLHEIDACKVPDWVSNTVWYQIFPERFANGNALLNPEGTLDWDSSVTPKSDDFFGGDLQG
IIDHMDYLQDLGITGLYLCPIFESTSNHKYNTTDYFEIDRHFGDKETFRELVDQAHHRGM
KVMLDAVFNHIGSQSLQWKNVVKNGEQSAYKDWFHIQQFPVTTEKLVNKRDLPYHVFGFE
DYMPKLNTANPEVKNYLLKVATYWIEEFNIDAWRLDVANEIDHQFWKDFRKAVLAKNPDL
YILGEVWHTSQHWLNGDEFHAVMNYPLSDSIKDYFLRGIKKTDQFIDEIN
>orf01408
(SEQ ID NO: 799)
MFNLLDSHDTERILWTANEDVQLVKSALAFFFLQKGTPCIYYGTELALTGGPDPDCRRCM
PWERVSSDNDMLNFMKRLIKIRKYASVIISHGKYSLQEIKSDLVALEWKYEGRILKVIFN
QSTEDYLLEKEAVALASNCQELENQLVISPDGFVIF
>orf01414
(SEQ ID NO: 800)
MSEQYRDIRKEVNLTADELKQIEKMMEVDNYRHFSPFVRDKILMTDDKQLAAKEWFSLWQ
SQKFEQISRDVHLVLIIARENHQVTQEHVSILLTCVQELIAEVNQVQSLSRGFREKYMR
>orf01415
(SEQ ID NO: 801)
MVYRYRTNLKKVFLTDSELHQLNERIAKSHCQNFSVYARKVLLNPNMSFVTINTDTYDQL
VFELRRIGNNINQIARAINQSRLISQEQLQELSKGVGELIKEVDKEFQVEVKRLKEFHGS
H
>orf01417
(SEQ ID NO: 802)
MVVTKHFATHGKKYRRRLIKYILNPDKTDNLKLVSDFGMSNYLDFPSYEEMVEMYNVNFT
NNDKLYEYRNDRQEKHQQNIHAHHLIQSFSPEDNLTPEEINRIGYETIMELTGGRFRFIV
ATHTDKDHIHNHILINAIDCNSDKKLIWNYALERNLRMISDRISKMAGAKIIEKRFSYRD
YQKYRATSHKFELKQRLYFLMQQSKSFDDFLEKAEQLHVHIDFSQKHSRFMMTDRAMTKP
IRGRQLSKRDLYDEDFFRMHFTKQEIASRLEFLLNCVNSLEGLLTKSKELNLTIDLKQKN
VIFILEENGKQFSLSHKKISDEKLYDVNFFQDYFKNKEVGVSEGIENLQAQYRAFQEERD
KEKVSTEEIEEAFETFKEKRDAVHEFEVKLTEHQIEKLVDEGIYIKVSFGINQSGLIFIP
NYQLDIMEEENQKKYKVYIRETTSYFVYNKEHSDKNQYIKGRTLIRQLTNDSRVIPYRRP
TVERLQEKISEISLLIELTETDKKYQDIKDNLVSEIAELDIKLTQTNEKIATLNKMAEVL
INSKSEGSGSQKLARHEFSKLNMTESTTLEQVNEELLKLQQEFGNVLDEYEKTIRKLGQL
FKVFDECINKEIMNEI
>orf01419
(SEQ ID NO: 803)
MVCLIIDVSPYSTLCDIVVPKTHFLRQLMELCDFSFIYDELEKNYQPDFGCRSYSLLIMM
FKYLLLKDIYKLSDVDVVERSFSGMTFKYFLGLAPVIEPSSLTKFRKLRNKDERLLDLLI
AKSVQIAIELGLIKSNILIVDATHTKVHYNHKKPQEVLRERSKALRKTIYQYSEYIKAEF
PSKPQEDTLVAELRYTQEVISVLEKHDELTGIPAISQNSITLKKL
>orf01420
(SEQ ID NO: 804)
LESSVKEEARIGHKSADSSFYGYKEHFAMTDERIITACVVTSGEKSDGPVLEELYHKSKD
NGVTIEAIVGDRAYSGKDNMQFTKKERVH
>orf01421
(SEQ ID NO: 805)
MSVFKFRIFGFYLVAMFGLFFKIGRFLKPLLENMFIALKGYQISLRLSPFFITAHF
>orf01425
(SEQ ID NO: 806)
MQEHYTPKGKHLTIDNRRLIERWKNENKSNREIAGLLGKAPQTIHNEVKRGTTLQQVRKG
LYKKVYSADYAQTVYQFNRKRSVKKLILTKEIREKILHYHKQKFSPEMMVNKKQVKVGIS
TIYYWFHNGHLGLTKADMLYPRKRKGVKKQASPNFKPAGKSIEERPDVINLRLENGHYEI
DTVLLTKIKNYCLLVLTDRRSRHQIIRLIPNKTAESVNQALTLLLGEHRILSITADNGSE
FKRLSEVFPEEHIYYAHAYSSWERGSNENHNRLIRRWLPKGTKKTTPKEVAFIENWINNY
PKKCLDYKSPSEFLLGG
>orf01426
(SEQ ID NO: 807)
MLHPIFIIRRSWDGIFHLSEWKRNEEFDFFNKMDVPYLSMSSRIEVTQAINFHKKHSISL
YAIISWCVMSAINSIPELLMDTDGKIVWQYNQRGCSFTTLTSEDKLNFSSFTMGDNLIEF
VSAFNINKQKAEEGQKPNIDKNNIAYLSCVPWIDFLHVSTPMNLSKIDTVPRITWGKVIQ
ENQRYFCTVNLQINHGMGDGLHVSNFFVLLQRFVNKINEYFQKK
>orf01428
(SEQ ID NO: 808)
MSIFIGGAWPYANGSLHIGHAAALLPGDILARYYRQKGEEVLYVSGSDCNGTPISIRAKK
ENKSVKEIADFYHKEFKETFEKLGFTYDLYSRTDSPLHHEIVQELFLQLYEKKFLYTKKI
KQLYCTFDNQFLPDRFVEGKCPNCGTHSRGDQCDNCSAILDPIDLVDKRCSICSNEPEVR
ETEHFYYVFSEFQNLLETYLNDAEETVRWRKNAINLTKRYLREGLPDRAVTRDLPNGIPV
PIDGFRDKKIYVWFEAVAGYYTASVDWAQKLQNNITDFWNNRTKSYYVHGKDNIPFHTII
WPAILSGLEIEPLPEYIISSEYLTLENKKISTSNNWAIWLNDIIKKYDADSIRYFLTINA
PEMKDANFSWREFIYSHNSELLGSYGNFINRTLKFIEKYFESEIPTKYLEGEILYNLKEL
YTTVGNLVESGHMKQALEEIFEYIRSANKFYDDMKPWALRESDIEKCKEVLATCVIIILN
LGQMLNPFIPFSGKKIEDMFKTKLNTWNYISNLPNKLSDVSMLFDRIDLKKIDEEVLELQ
QTSSR
>orf01429
(SEQ ID NO: 809)
LNNLTLLKEYNFRDLGNHLTQTGQKIKPKTLFRSSKLFGISKIDVDLLQSYGITKVIDFR
SANEIKKAPDPDIKNIKNIVIPIFYNDDSELTEFPIEFFNKSDAGFQHMIKTYDQMINQK
QSKLGYKKFFKLLLSHPKDESLLFHCSMGKDRTGIASLFLLYILGVDMNDIFHDYLLSNK
YLINVRKENIEYVNNHSGNVILMHNLLSLSSAKEEYINRVLNVLDKEYGGILRYINTELG
ISSQEIEELKDRYLF
>orf01431
(SEQ ID NO: 810)
MDFLNEVLDLKEFIQDPVRTLSLGQRMRADIAASLLHNPKVLFLDEPTIGLDVSVKDNIR
RAITQINQEEETTILLTTHDLGDIEQLCDRIFMIDKGREIFDGTVNQLKKTFGKMKTLSF
ELHPGQDYIVSHFEGLSDIYVTRQELSLDIQYDSSQYQTADIIQQTLSDFTIRDLKMTDA
NIEDIIRRFYRKEL
>orf01432
(SEQ ID NO: 811)
MTKLWKRYKPFVSAGIQELITYRVNFFLYRIGDVMGAFVAFYLWKAVFDSSHQSLIQGFT
LSDMTLYIIMSFVTNLLTKSDSSFMIGWEVKDGSIIMRLLRPVHFAMSYLFTEIGSRWLV
FVSVGLPFVILIAGLKLLSGESFLQIVLITTVYLLSLILAFLINFFSIFALVFQLLCLKT
YGDQIF
>orf01433
(SEQ ID NO: 812)
MKKYQRMHLIFIRQYLKQIMEYKADFLVGVVGVFLTQGLNMLFLNILFQHIPLLDGWSFH
QVAFIYGFSLIPKGIDHLFFDNLWALGQHLIRKGEFDKYLTRPISPLFHILVETFQIDAL
GELLVGVLLLLMTITSLTWTWAKVFLFLISIPFATLIYTSLKIVTASIAFWTKQSGAIIY
IFYMFNDFAKYPIAIYHSFLRWLISFIIPFAFTAYYPASYFLKDKDGLFNIGGLILISLI
FFTLSLKLWNKGLDAYESAGS
>orf01434
(SEQ ID NO: 813)
MIELAEPLPEYEILLSIPGIAETTATSIIGELETFVAFSLPTKSMPLSVLTSDTMNLAIS
>orf01435
(SEQ ID NO: 814)
MLGWKDGHEVPILFPCRSREKVLYFWKGNLKHLVQAILSPNDVFCQILIESTEVTQILID
FFLNISWFAVKDEL
>orf01437
(SEQ ID NO: 815)
MMRTVFRMDVSKASSEVAILVNGEKVHGYTMPNDAIGFSRLLEDLK
>orf01438
(SEQ ID NO: 816)
MKKNNVEIIKADSLVRRRGDNVERHLKRVAAYCRVSSDSEDQKNSYDSQVRHYKEYISQR
SDWELADIYADEGISGTQVGKRQDFQRLINDCANGEIDYIVTKAIARFARNTLDTLKYVR
MLKDMQIGVYFEEENIDTLTMDGELLLTILSSVAQQEVENTSAHVKKGLKMKMQRGELVG
FQGCLGYDYDVETKQISINKKEAKIVRYIFERYLEGIGGKVIARELDELGYKSPRGLEHW
NDTTVLGIIKNEKYKGDILMGKTFTVDPISKRRLSNFGEEDKYYIKDNHEPIISKEDFEK
AQEIRLRRAGNKKTAANVNGKRERYSKMYAFSSMLECGFCGSILSRRSWHCRSDYRKVVW
HCVTSIKKGKKFCKHSKGLEEIAIEGAFLEAYRQVYHSNENLMTDLLETIESELNDNSLN
KELKRITNKLRILLKKEENLVNLRLEGKVSDSIYNEKYNEISSEKEFLAEEKVNIETTLK
SEIDVKKRLTEFKHLLSSQKMLTEFDRAVFESIVEKIIVGGVNSNGEIDPAMLTIIFKTG
EIQNKDGKQFKSKRKNAKLETDKLCPQNSDEDKKLYSQGTDNTRGVCSVAGSILASQ
>orf01439
(SEQ ID NO: 817)
MGGNPPMKKYSIVDKIVLSTKIKRIIIFTVFRENWEPYMKKYTEVFQSQFPNLNIDYLLL
DTEQIDLDSYLDADIIIIGGGNTEKYIATYVNQEFKSYIDHMLNKEAKIIGFSAGALLLG
EKVYVSPNDNSDHQIKIKNGLGLFSQFLISVHYDSWNDKANKDRAEELVNVPIIPLNDHS
CLVLDKLGNIIEKID
>orf01440
(SEQ ID NO: 818)
MDDEASKQLSDSRFKILVGVQRTTFEEMLAVLKTAYQRKRAKGGRKTKLSLDDLLMVTIQ
YMRE
>orf01457
(SEQ ID NO: 819)
MGFSMKLIHDLDTHTTHSTAKMLYNVKAIKNDFSIRE
>orf01492
(SEQ ID NO: 820)
LILNEYEKRIFHEKTHNIECFDTCYYAFFIIFAPFLAFVIDKHCSSSLFLER
>orf01520
(SEQ ID NO: 821)
MSQVKGLCVLDVDGTLILEEVIDLLGREAGHEAEISQITSRAMRGELVFESSLRKRVSLL
EGLPILVFDNVFNSIHLSLNVPEFISILQKNGILVGLVPGGFTPIVGEISKIPWYCLFHC
QPA
>orf01521
(SEQ ID NO: 822)
MLKSAELGIAFCSKEMLKKEIPHHVDKRDFLEVLPLIDCLE
>orf01527
(SEQ ID NO: 823)
MGFSMKLIHDLNTHTTHSTAKMLYNVKAIKNDFSIRE
>orf01537
(SEQ ID NO: 824)
MDKLIIFIEKGKPFFEKLSRNIYLRAIKDGFISSMPAVLFSSIFILIAAVPNIFGFKWSD
EQLAFILKPYNYSMGILALLVAGTTAKSLTDSVNTRSMEKTNQINYMSTFLAAVVGLLIL
AADPIEGGFANGLLGTRGLLTAFLAAFITVNIYKVCIKNNVTIRLPEEVPPNIAQVFKDV
IPFALSVLSIYGLDLIVRNIFGTNVAESVGKILAPLFSATDGYIGLAIVFGAYAFFWFVG
IHGPSVVEPLIVAISYANIEANVQLVQAGMHADKILNPVTQTFVVTMGGTGATLVVPFMF
MWLCKSKRNRIVGRASVVPTFFGVNEPILFGAPIVLNPIFFIPFVTAPIINVWIMKFFVD
VLQMNSFSIILPWTTPAPIGIVMGTALAPLSFVLAITLIIIDTLIYYPFVKVYDHQILEE
ERKGNSSSELKEKVAANFNTVKADAILEKAGVDAAQNTITEETNVLVLCAGGGTSGLLAN
ALNKAAAEYNVPVKAAAGGYGAHREMLPEFDLVILAPQVASNFEDMKAETDKLGIKLAKT
EGAQYIKLTRDGKGALAFVQEQFD
>orf01552
(SEQ ID NO: 825)
LFKTRSNSSALGSSYISNRNIFSYFTNQFNNTFCNVFGM
>orf01557
(SEQ ID NO: 826)
MALTQRQFVELFQETINVITLTCLTVSVAVVACVSICSS
>orf01558
(SEQ ID NO: 827)
MLTMFLFLPIDFFFCTDIIRMSCILKVNIVFSIYLNHITTLDFTDNILVL
>orf01560
(SEQ ID NO: 828)
LVCYFDDDLFGIDSFTLANLIRSQILRFLRRLFSIYIGNTIISLTVLA
>orf01570
(SEQ ID NO: 829)
MGFSMKLIHDLNTHTTHSTAKMLYNVKAIKNDFSIRE
>orf01585
(SEQ ID NO: 830)
MEKYFGEKQERFSFRKLSVGLVSATISSLFFMSVLASSSVDAQETAGVHYKYVADSELSS
EEKKQLVYDIPTYVENDDETYYLVYKLNSQNQLAELPNTGSKNERQALVAGASLAALGIL
IFAVSKKKVKNKTVLHLVLVAGIGNGVLVSVHALENHLLLNYNTDYELTSGEKLPLPKEI
SGYTYIGYIKEGKTTSDFEVSNQEKSAATPTKQQKVDYNVTPNFVDHPSTVQAIQEQTPV
SSTKPTEVQVVEKPFSTKLINPRKEEKQSSDSQEQLAEHKNLETKKEEKISPKEKTGVNT
LNPQDEVLSGQLNKPELLYREETIETKIDFQEETQENPDLAEGTVRVKQEGELGKKVEIV
RIFSVNKEEVSREIVSTSTTAPSPRIVEKGTKKTQVIKEQPETGVEHKDVQSGAIVEPAI
QPELPEAVVSDKGVPEVQPALSEAVVTDKGEPAVQPELSEAVVTDKGEPAVQPELSEAVV
TDKGEPAVQPELPEAVVSDKGEPAVQPELPEAVVTDKGETEVQPESPDTVVSDKGEPKQV
APLPEYTGPQASAIVEPEQVAPLPEYTGVQAGSIVEPEKVEAPKEYTGKIEQPSAEDTKP
ENEASSTNGESERPKDKIKEEKQVDKKLELRNVSNVELYTVENNKYRHITAVDGALDSSL
KYFMKVKSENFKDIMLPVTKIESTTKNNKEVYKIVAHAENLIQHENNVISNDYTYYLPKT
QQSETGVYTSFKNLVDAMNSDPNGTFHLGATMDAREVELPDDQESYVKNEFYGKLIGENN
GKYYAIYNLKKPLFKTLNTATIQNLSIKEANVSSKEDAATISKEAKYNTLIDNVHSDGII
AGERGIGGLVSKVDNSRISNSSFTGRITNTYDTTAGYEIGGLVGKLSGSLASIEKSIASI
DIASNAKSGDQIVGGIAGVVEKSATIKYSYVEGNVNNVRHFGKVGGVAGNLWDRDSQDVS
KSGKLSYVLSDVNVTNGNAIAGYNFNGIKTIETYSNKNNKVVNVVQEDDEVVTKDSDVQR
GTVLDADKVKEKKVELVSKHSTKVEDFDFTSRYNTNYNEVTGYQQSREQVYKNIEKLLPF
YNRETIVKYGNLVEDNSDLFTKKLLSVVPMKNNEVITDINKNKQEINKLLLHFEGNKSRV
LNIAYKNDFSKVAEYDIANTKLMYTPNTMLHDYNNIVKTILNDLKSVQYSSADVRKVLDI
SGNIKLTELYLGEQFEKTKANIEDSLSKLLTADAAIVENNNKVIDNYVIEKIKNNKEALL
LGLTYLERWYNFNYGETNAKDLIMYHLDFFGKSNSSALDNVIELGKSGFNNLLAKNNVIT
YNVLLAKNYGTESLFKALEGYRKVFLPTISNNEWFKKQTKAYIVEEKSTIEEGREKQGKE
GTKYSIGVYDRLTNPSWKYQSMVLPLLTLPEEKTVFMIANISTIGFGAYDRYRSSEYPKG
EKLNKFVEDNAKEAAKRFRDHYDYWYKILDNDNKEKLYRSILVYDAFKFGTDKDKDKVTH
QATFETDHPAIKYFFGPAGNNVVHNGHGAYATGDAFYYMAYRMLDKDGAVTYTHEMTHNS
DREIYLGGYGRRSGLGPEFYAKGLLQAPDHPYDPTITINSVLKYEDSENSTRLQVADPTQ
RFNSAEDLHNYMHNMFDVIYMLEYLEGKAVANLETNQKYELLRKIENKFDLDQDGNNVYA
TNVVRRLTMDEVNKLNSFDSLIENDIITSRGYKDQEYKRNGYYTIDLFSPIYSALSGEKG
TPGDLMGRRIAFELLAAKGYKEGMVPYISNQYEKDAKAAGSKINSYGKEVGLVTDELVLE
KVFNGQYKTWTQFKKDMYKEREKQFSKLNRVNFINPNNPLSRQRNVSVTDIGVLERMIVE
AVRDDAQDDVAKFYPETNSRVLKLKKAIYKAYLDQTNDFRSSIFENKK
>orf01588
(SEQ ID NO: 831)
LSLLKKDKFSIRKIKGIVGSVFLGSLLFAPSVVGASTYHYLDYSSLTQTERDQLKQGRPD
ESKESYALDYEKDALPNTGSSQSIMTALGLLAIGSLIVIITKDNRNKKIATFLIVGATGL
VTLSTASALNLNANIHESGRDGVLQISGYRYVGYLELDDKTVSSVSPASTVSPVEQPKVV
TEKGEPEVQPALPEAVVTDKGEPEVQPTLPEAVVTDKGEPEVHEKPDYTQPIGANLVEPE
VHEKLAYTESVGTTGMDENGNLIEPPVSDIPEYTESVGTTGVDENGNLIEPPVSDIPEYT
ESVGTTGVDENGNLIEPPVNDIPEYTEPISTVSEVASEREELPSLHTDIRTETIPKTTIE
ESDPSKFIGDDSVKEVGEDGERQIVTSYEELHGKKISEPVETVTILKEMKPKILVKGTKE
NPKEKTVPVLTLTKVTEDAMNRSANLNYELDNKDNAEISSIIAEIKDGDTVVKKVDLSKE
KLTDAVQNLDLFKDYKIATTMIYDRGQGSETSKLDEKTLRLELKKVEIKNISSTNLVKVN
DDGTEIPSDFMSEKPSDEDVKKMYLKITSRDNKVTRLAVDKIELVTEKEKELYKITASAQ
DLIQHVDPSKTRNEYIHYIEKPVPKVNNVYYNFNELVRDMQEHPNDEFKLGADLNATNVS
AFGKSYVTKDFKGKLLSDGDNHYTIHNLSRPLFGNVIGGTIKNINLGNVDINMPWANQVA
AVANIIKGGTTIENVKVKGNIVGKDWVSGFIDKIDNQGTLRNVAFIGNVTSVGDGGQFLT
GIVGENWKGLVERAYVNANLIGKKAKAAGIAYWTQNEGNNNTVRQEGAIKKSIAKGTIQV
TEAIESGGVVGSMKHHGSVEDSVSMMKVPNGEIFYGSSDIDYDDGYWTGDNVRRNYVVIG
VSDGHSSYQRSKDKNRIRPISEEEAKSKIEATGITADKYEINEPVVNRLNRLTRREDEYK
STQDYKVDRDLAYRNIEKLQPFYNKEWIVNQGNKLAEDSNLAKKEVLSVTGMKDGQFVTD
LSDIDHVMIHYADKTKEIKAVHQKESKVAQVREYSIDGLDDIVYTPNMVDKNRDQLIKDI
KDRLATVELISPEVRALMDKRDTSRDPNANSDERKNGYIRDLYFEESFSETKANLDKLVK
SLIENADHQLNSDEAAMKALVKKVDENKAKIVMALTYLNRYYDIKYGDMTIKNLMMFKPD
FYGKSVDLLDFLIRIGSSERNIKGDRTLDAYRDMIGGTIGKSELHGFLDYNMRLFTNDTD
LNDWFIHAAKNVYIVEPKTTNPDFVNKRHRAFDGLNNGVHNRMILPLLTLKNAHMFLIST
YNTMAYSSFEKYGKYTEAEREAFKDKIKEVAHAQQTYLDFWSRLALPSVRDQLLKSQNRV
PTPVWDNQNYHNVEGVNRMGYDKNNKPIAPIRELYGPTWRYHTTNWYMGAMASIFQDPNN
NDQVYFMGTNMISPFGISAFTHETTHVNDRMLYFGGHRHRQGTDVEAYAQGMLQTPDKSG
NGEYGALGLNMAYHRENDGDQWYNYDPDKLETREDIDRYMRNYNDALMMLDHLEADAVIP
KLHGNISRWFKKMDRQYRKNGELHQFDKVRELTEDEKKKIVINNIDDLVNNNLMTKHGAP
SDRTYNPEDFDSAYVNINMMTGIYGGNTSQGAPGAASFKHNTFRMWGYFGYENGFISYAS
SKYQGEADKTNKKLLGDDFIIKKVSKDKFNNLEEWKKQYFKDVKSKAEKGFTAIEIDGRQ
ITNYAQLKTLFAEAVQKDIDGMSDPKIKDHFKNTVDLKSKVFKALLKNTDGFFNKLFKED
I
>orf01603
(SEQ ID NO: 832)
VLGGRANSVTSCTTNSHWNLTFTTKHVTCFSSLVDDIVHGNNREVHEGHIDDWTKSCHGC
SCCCSRDGSFRNRTVTDTFWTKFFKHSNRSTEVSSEDTDVFSHQEHIFIATHFLRHSKDN
GVTEGHCFCFHFISFSLVCVNIFKG
>orf01604
(SEQ ID NO: 833)
MDMFYIGHFLDIRRDTVTVVNAIENDWQVPDRSHVHCFVENTFIGRTISKEADNDFTGIL
HLLTEGCTDSDPHTTTYDTIGTKVPSIKVSDMHRSTFPFTGSSVFTKDFSHHSVEVNPFS
NSLPVSTVV
>orf01606
(SEQ ID NO: 834)
MNXXDFIGHCDKIKRNIFEKSHKVFSGLFGLHPKDFLNLIFSNQIPLPFSECNPLTNYNH
LFSLIISDKRDIVIHWI
>orf01622
(SEQ ID NO: 835)
LIEIQVFSSLQVCCLNLCHLKFQHFDTCLVFLLVFLDFQNLLAHFPIGIKTRLIGFFQVP
KSGITKFIQHLDMQLGTH
>orf01623
(SEQ ID NO: 836)
MVMLTMNIYKMLPNSSQNRQINHLTIYTADTTTILQDFPTDDNFIT
>orf01624
(SEQ ID NO: 837)
MTNNICRRTSSQHHIHGINDNRLPCTRFTSQDSHPLFKIEGNSLNNGKVFYRNFK
>orf01634
(SEQ ID NO: 838)
LFVIRNPSSQTLFQTQLQLVQALQITVIQALRLSKDNRLTAFFQSLLFLR
>orf01636
(SEQ ID NO: 839)
MPHTRDNWQTRFKNSSYHNFFVKGPEILNRTTSTTNNEQIQIVPLISTRNISSNFLRSPF
TLNLGRIKKDVNTWESPADGRDNISNNGSTTAGYYPNSLRKLGQSLLEAFLKQAFFCQFF
LKLFKLNRKRPNPIRLNFFNDDGVATTWFIDLYTPNHIDLHSFFQVKP
>orf01637
(SEQ ID NO: 840)
VTLADFVADRRAATPTAAAELATPVTKVGCISSFAKSGKTDGNGSPKCSI
>orf01640
(SEQ ID NO: 841)
LAIIRNRTCSLKLINDHLTFWTLRFLTSTRILIELATINLNCRIHRGNLSNRPSQASNRF
INKLFIQGRQNRGFCDHFPTSILSRRGIAQSDFPLIDLTLVLHKLDHACRLANRNRQNTH
HIRIQGSTMTNFLGSQNLTQFKNRIMRGHSCFLF
>orf01642
(SEQ ID NO: 842)
MEDDLNYENLMDDVTEAIKKFNLVIFIGAGVSIAQGYPNWNNYIEHLIKYWQGQVLSVSG
EKRLGREHHVVFDLISKSSISNKRKVDLVNYELKKVFGEDFEKRRLDFEKGYFKNLLPYS
IVNQTVESLASLNAIFITSNYDYEIENHIKRLKNAVVTINDLNEFTKNKNGKLQFGDVLH
IHGTPDCDVKYFVSSSADYSKTYLKNRENFENLVTWFKETKPTVLFIGAGLEEDEILSLL
CKDSKNYALMKSENTGNQRVDEHYRGVVEGFFSSENHTQIIWYGDEFEKLPLFVKKLVAD
INEKLGTHDFYNQWNNLLNPSINQEEYNKNLDSISNDFKYLSSVLDKVIENDNNQLDQLM
LNALLRSETLTVIKKNFVLVFWKFIVKNIEKLSDNEWDVIYKIIYEGSQNYFIDDVFFVY
NYAIDNKISSFTNNNKLNELREIISKDGYIVNSNFNKDKTLLGYWLVSAFEQQNRDLYIK
EDSEVEVNLNYECVNKLMSILNNPEFLSYNYYSIEHQLKEYDVVKFLYELVKSKKLFIEE
EKFLESDSEDLISTILIQKLLVQLDNEINLDLEFIKRLIDKIDFSNIHFGEELNTFIKEH
RSIIREKNIEIPKKPYRNWISSLEGGFVSQFSYLTQENLVEYDESRVLEILVNAEKEQRG
SSFLEEKTINETENFFITVLKESNEISKKVSDLLKNHIDDLYPKYKRLYVKIISFPEIEE
NLRKIVREKYLKRFNKESFDSNDRKFFEYHIKQQNTDIDIFEKLLSINVNELSTPKGDNK
QLDILHFINSEMGSYFQCLISLFINHSSYRDVIIQIINSVTDTDYREFAQGILLNEYNPN
RINVTYNTFLGFAYYHSTITIEAADVFTDVVRDILNKKIEDNQILNKVYLVALERVDPTI
ESFSLSKNNYSQMINIIFTGDYEFRYSKEWLGALFKFDSSANYLVTIFYLLYNENLKKNR
FALFIEELSDYLTTYNQKLSLRGMNYKLNHEELNNFDLLKKMFLKLMETDKIENDIFYLD
GIKSILPLLSLDDRRNVLQHIQKQNNCPPPEIEELQRIIVN
>orf01645
(SEQ ID NO: 843)
MFMSNLCQFFQVWNINQGVTQGFNQDKLGIVFDSCFYFLQIINIDKGCCDTITRKEFFQK
IEGSTVNSRSSHYMVTSMGKRQNRISHCSHT
>orf01646
(SEQ ID NO: 844)
LINVFSHGVDIAIHSATKFIGGHGTTIGGIIVDSGRFDWMASGKFPQFVDEGSSCHNLSY
TRDVGAVAFIIAVRVQLLRDTGAALSPFNAFLLLQRLETLSLRVERHVQNAETIVDFLVN
HPKVEKVNYPKLADSPYYALAEKYLPKSVGSIFTFHVKGGEEEARKVIDNLEIFSDLANA
ADAKSLVVHPATTTHGQLSEKDLEAAGVTPN
>orf01647
(SEQ ID NO: 845)
MTCDFKFETLQLHAGQVVAPATKSRAVPIYQTTFFVFDDT
>orf01651
(SEQ ID NO: 846)
MAWLLVGNVGVRQVLEHLNAELKKVMQLSGTQNIENVKPFNSVTSIKPTLPNDPPDLKFI
DKKNAPPKCGVFLCYGKKFVLKNKKNEIRIGRIVQDSQIDF
>orf01656
(SEQ ID NO: 847)
LKKKWFFADYYDTTIILLALISVILVLLGFAEMIDLDNPPYSIIDLVIWGVFVIDYSWRF
FITKRKWRFILENVFDLLAILPLNAIFTVFRLGRIFRLARLTKLLKLTRLLRIIGLTGKL
ERKISRFLRTNGLIYILYVNIFIVLVGSSILSVVEEKSFSDSLWWALVTVTTVGYGDIVP
VSLFGKTNYRAKEY
>orf01666
(SEQ ID NO: 848)
VVDFKQTRQDPHDITIYSWLRQVKSNTGNGSCCVRSNPFQAGNSFIGIWKLATKVSHNLL
GCSLHIANSRIITQALPSFQ
>orf01671
(SEQ ID NO: 849)
MAERTVVQVHNAFPEDTTLINSQLIPLVQVVVNQGRKGIVGSCNSMHISSKVEVDVFHWQ
NLCIPTTSSTTLDPHDWTKRRFADSNHGFLANLVQGIRKTNGKRRLSFTCRCWVDGSNQD
QFTDWIALNCTNFIKAEFSLVLSVQLQIVVRNTKFLYNINNWLQLNTLCDFNICFHSKFL
>orf01684
(SEQ ID NO: 850)
MLFIIGHLNFPTAGSFIDSTLHRLGNRVCIHDDMAFTVTSSTSNSLDESTFVAKETFLVS
IENSYEAHFRNVNSFTEQVNSDQDIKDTQAQVTDNLRPFQGLDIRVHVLDLDTHFLEVVG
QILCHFLGQSCDKGTLIFFNAGIDFTQEVINLSHSRTDFHLWIQESRWTNDLLNHCLGLF
IFIVTRCR
>orf01685
(SEQ ID NO: 851)
MNVTLKLLPTERTIVQSRRQTETIINQHFFTRTVSIVHALDLPYGHMTLVNHNQEIIWEE
VEKRIRRLSFAPSIHVARIIFNPIGIAHLTQHFDIILCPLFQTLGFKQFTFLFKDS
>orf01686
(SEQ ID NO: 852)
MIHFSQHLTCQSLNFTNTVNFVSKKFYSKGMFISGSWENLYHIPTNAKSSALEINIITFK
LNIDQVIQEFITRNL
>orf01687
(SEQ ID NO: 853)
VAKLVNLVIDRTILLNIGIARRDIGLWLVIIIVGYEILNCIFREKFLKLPIELTSQSFIV
GNNQSWFIDFRNDLTHSIGLPCSSRPHQNLSFFSPLNVIHQLLDSLGLIS
>orf01705
(SEQ ID NO: 854)
MNITQTDFLAVNLVFAISTTIDMAFHPDFLTCILDKSVMIIQSHNYRSIIERFATFCSSK
DDIRHLAPTETLDTRLPQSPTQTFCNIGLSRSIGSNNCRHTLVKNDLGLISKRLEPLNFD
FL
>orf01707
(SEQ ID NO: 855)
MSFIVCNHLKFACFNLRNHDLIDKFLDLGHILIQKKGTKKGFKGITKNGVTIATTRFFFP
FTQLDKLVKLAITRKTS
>orf01719
(SEQ ID NO: 856)
LFTCFSKLDNKTASTTYISHKFFTAIPVCFEFFKGFWFPRKDTTKKNIFIPMFLVECFNF
WVELR
>orf01726
(SEQ ID NO: 857)
MTKKIVALAGDGIGPEIMEAGLEVLEALAKKTGFVYEIDRRPFGGAGIDAAGHPLPDETL
KACREADAILLAAIGSPQYDGAVVRPEQGLLALRKELNLYANIRPVKIFESLKHLSPLKS
ERIAGVDFVIVRELTGGIYFGDHILEERKARDINDYSYEEVERIIRKAFEIARNRRKIVT
SIDKQNVLATSKLWRKVAEEVAQDFPDVTLEHQLVDSAAMLMITNPAKFDVIVTENLFGD
ILSDESSVLSGTLGVMPSASHSENGPSLYEPIHGSVPDIAGQGIANPISMILSVSMMLRD
SFGGYEDAERIKRAVETSLAAGILTRDIGGQASTKEMTEAIIARL
>orf01727
(SEQ ID NO: 858)
VVRNTASHLTCILFLNKGISVYIGNSRSLKHIKIKPCCIKDGFCISVFNSDQNPILGIDS
ICYRIDSVGHQTNRLVKELIDSIKDCFNGTLPCRIKFDFLTIHIG
>orf01729
(SEQ ID NO: 859)
VEAFWIFNHGSSYQSSNICICDFLLLIGQCLELSKEWFDILFCKI
>orf01732
(SEQ ID NO: 860)
LANIESHCNFFQSSISSSLPNTIDSPFNTSCTILDSSKAICHCHSEVIMTVRRIDDLTIR
LDILNQVFEDGTIFL
>orf01741
(SEQ ID NO: 861)
MQEHYTPKGKHLTIDNRRLIERWKNENKSNREIAGLLGKAPQTIHNEVKRGTTLQQVRKG
LYKKVYSADYAQTVYQFNRKRSVKKLILTKEIREKILHYHKQKFSPEMMVNKKQVKVGIS
TIYYWFHNGHLGLTKADMLYPRKRKGVKKQASPNFKPAGKSIEERPDVINLRLENGHYEI
DTVLLTKIKNYCLLVLTDRRSRHQIIRLIPNKTAESVNQALTLLLGEHRILSITADNGSE
FKRLSEVFPEEHIYYAHAYSSWERGSNENHNRLIRRWLPKGTKKTTPKEVAFIENWINNY
PKKCLDYKSPSEFLLGG
>orf01752
(SEQ ID NO: 862)
VHAHTDKLCNGCNRIFNSIISHHTIFRERNKLSHKAIKSTRQEMGPCHVVFIEFFITLHR
RLIGNHDNFLTNLVGSGRVRNDGST
>orf01753
(SEQ ID NO: 863)
VNHCHWELFIQNLGITFSLIVTLIRMTDSHVVGTDKDMIFLVNSLFLIFDIDKLRLS
>orf01755
(SEQ ID NO: 864)
VGNNDILWSKRTISINGFNDFLNTCIAVSTTLCNDDTFLIKRKIFIYKIFCMRNPVSMNT
NYNFFNTWLQDKFFNCMNQNRSIT
>orf01765
(SEQ ID NO: 865)
LVAPVASSTRFFKNNDSLTSWNNGFIIITINTIISYQRISKGQDLSIIRLVCNGFLVAGH
PCIKDDFACYINICSEGLAFKNCAIF
>orf01767
(SEQ ID NO: 866)
VVCYFYITIDWSWVHEDCCFFQTIVTFLSQAMLGMVVFF
>orf01768
(SEQ ID NO: 867)
MAFVLHTEKHHDINLINDFINGYKLSIVCKLLTSPFLRSSEKEFRSQAFQNLHIGFGNA
>orf01769
(SEQ ID NO: 868)
VVQVTCNSNFKTLKVAKFLINGHQIKQALARVLARTISTIDDGSRNRWTSNQFSIVVDLW
MANHTDIHS
>orf01770
(SEQ ID NO: 869)
MCPCRILKEEIGNNRMVFIGKLGSIFKLNSSLDQFHYLIDSEVFHGHHMVQCLLIF
>orf01776
(SEQ ID NO: 870)
MSINCKGWNPKSYTHDNIGCLATNTCQTLQFFTCLRDLTIKIV
>orf01790
(SEQ ID NO: 871)
MPDCTLTNFLDKVLYNRQGNVGLEQGQANFFGCLLDIRFRDFSFFT
>orf01793
(SEQ ID NO: 872)
VQFHLIIFQNLFCSLDIVIDSLTTDTELLGNFSKAVIISVVELDIIHLLICQKRRIKFKE
RIHTIGFFDFHNFYYTKN
>orf01796
(SEQ ID NO: 873)
MKFNHYFFLFLIIEKQVAIISFFMHFHIIKLVNHFQLLIKLNCISHPNLHIRPSFLSLVL
LFYQKEQDFAIMVI
>orf01799
(SEQ ID NO: 874)
LANNRKTETLGVSYLSTFIDKHELLQSYFESNDKTPVWDGEIHVLKSPSEKKDEILGKVP
VQIKTTRQKKDVLKSFSLDTRDLELYKPNGGVVLFVVWLNEDNGLRDIYYKSLPPLSIKN
LLKKSKLKNKSTNRKKLSIEIFKLDEKKMYPMLVDFINNSQKQYSFINVEGISVEDIPDD
KTLKFYFYGQEKEEIFNYQEEHDLFIYYLDPITGIEIPLENTIKIVETEEETDLIIKIGD
YVFQDVKRHRFPDGSVQLHFGESFTMSFDIKKKQFKFNYTRPDLLSKAIKCTQVFQELGK
IGYFTLNGNKIELDERSIKDISSLDLEADIKGLLKISNFMKKMGIQKDVDLSCFDKQSQR
NLNILYSGLVLKKKVALNYNESKLLHLNIANIHIITLYSFLSDKNGTMIDIFTETPWCRE
GETEDEDYLDISIFEVFEPNDWLKIDNCKIDSVIASYQRLVDNKLKYEGADRTILKIVIA
ADMAEDKTKRELLLNWAQCLSDWNLKYSKNCEMAIINDLQIKSRVRKLNSKETETLTNIL
VNSNDNYELCFGSSVLLKSKPQADLFWNKLDNETKERYKDFPIYTLYMKLS
>orf01800
(SEQ ID NO: 875)
MKVSKKITLFSLSFAGFVLLTLPQAGKAFELKEDWAFKGGIRYENGKVSKINNGYEVNIK
VLDLPSTSAIEWTVRLNGEKQNTNFLAEERTVSKTEDKGRFLHFYIPYGYRGDIVVEAKS
GNEVKTWSTKVVDDVYSDSAKSGYFILDGEQILESSWDSVNESYIATLPTVTSGKTVVAW
REKGTLNLIKPGRIARQYNSSGSYVELSPIFETASWLKSNQNWYYQKQGQLVQNSWIKDQ
GSWYFMDDEGVMFNQTWLHQGGSWYAFKSSGAMISADWLYDNGSWYYLKDSGSMVTGWLK
NGGSWYYLNKSGSMATGWIKDSGTWYYLKNSGSMATGWVKDSGSWYYLKNSGSMATGWVK
DNGKWYYLASSGNMLRNTRTPDGYYVDGSGAWK
>orf01801
(SEQ ID NO: 876)
MKKILLSTVALLSLVASLLANNPVSAQESSSQATYSKSSGSWIKSGNRWWYKHSDGSYTT
NGWEKINGTWYYFDSEGWMKTGWIKEYGKWYYLDDSGAMKTGWCLVSGSWYYLNSSGVMQ
TGLQTINGKQYYLAAGGAMQTGWHNIGDDTYFFANSGENQNINRRALVLGETSTRAVPIA
DVNAMEKVFNNQNFSEVVRFPDRTKSEIIAKMQELFESSSEGDVNYLYFTCHGGRDGRIY
IGSDGLAFSGWELASVLKQYKGKFVVMLDCCHAGTIISKDNTGEGNEGASTEYFDLDEFV
SGFSNMDGNEKSGEMIDSKFLVLCSSRGAEYSSGGSLSLATKYWSLGSGWNPLQNSQAYL
AADQNNNRRITLNELYTYSREQVLKQNSNQHIEVYPDNSQFVLFKK
>orf01802
(SEQ ID NO: 877)
MENFGAVLKDIRISKNFRLKDLSCNEISESTISRFENGITKLSINHFYILLNRLGISFSE
FEELVHCYYSKKECLFEELEHAVNSSDIFLLQELVDKIELKQKQEKSLCNYHIKLIAEQQ
INRLANLPYNSSKCNELIKYLLSVDTWHEYELKLFYNSVFFMNTRTISLLYRIVIKKTRY
FLKTNTGTHRIIPLYLFNLKLLLKNNLLGSAQFFIDDLENLLTRQGYYFEKNYLLFLKGI
YLIKTNQIELGKKECFKAMRIFKEYNDSDTINELNQKFKLDLTI
>orf01803
(SEQ ID NO: 878)
MSSIYSSAKKDFLYWNVLIFIMELPNDVKVQFYELRKKVQSFNQLSKRFGMDVSG
>orf01810
(SEQ ID NO: 879)
LSFLILSPAGAQESLSFFFVKITDASKTVKNGGQTETQKLVTKMASDFERVENKDSEVGK
IVKEKLALSGDITEAKLTEISSALLAFEKEQNPVDLDAEKEKLVNRLSPRFETLEQAIAS
KDLEKVREAFKKMNSTWTINESVVRDNSTAHYGRVETAISFLPSSMETEPTDESGT
>orf01812
(SEQ ID NO: 880)
MQKNIYFVVLDLHTTDRDKIIQLFKDWTDYSAKLVEGELVKKDGQNALFPPSDTGETVGL
NPHRLTLTFGVSASFLKRMNLENKRPRLFRDLPLFPKEQLREKYTGGDIVIHACADDEQI
AFHAIRNLIRKGRNAVPLRWSQSGFAAIGDRMETPWNLFGFKDGTANPTKEQDFDRVIWA
DSKDWMENGSYMAVRRIQMFLETWDRTSLEEQENTFGRYKESGAPFGKNNEFDEVDLSLL
PDDSHVCLAKEVDKPLLRRSYSYSDGIDEKTGQFDTGLLFISFQKDPDNFVKVQTNLGAT
DKMNEYITHIGSGLFTCFGGVEKGGYIGQKLLEG
>orf01815
(SEQ ID NO: 881)
MTGKKGFLFLNCHICMVTTTICFLKERVESELLIFFYISLNRCLITV
>orf01818
(SEQ ID NO: 882)
MSLRNKIEQHIKELEGGKFQKLGDAYLSRKYNFNIVSLGSQEGTDKTTKGIPDSYAVENG
KYVYIMYGTHKSVISKLEGDIQSVKKKILEENIAEDKVGRLICCHTSSNITIKQKEDLEK
MAEPYHLELIGINEIANDLTKIDFQYLAKEYLSISESTEQVWSINDFIRIHDESKTNAPI
SNDYIGDVSEIINTIKSSEKRIFLISAKPGTGKTRLAIEICSLLDRNKYNIICVKSNNQD
IYQDVKRNLNLHKENIVFIDDVNTIQNYISTLGLLNTTSNIRFILTVRDYAKKDVINNIK
VYGYNNIEPELIKDDNFKELLNQFSRNDFTNQEIEHIKTISKSNPRIAVIAAKLSSSQDL
TNFNDEIDILKDYYEEILNKNNIIYAEQKTLFILSYLKKIRLESLEENQEFNKLLKITDI
TNTDFKSAVEKLHERELCNIYNDKIVKIADQSLDDYIVIKFLINKKISILEILHELYPVN
DQRVVQILNQCSNFIRKESDLEGVSDAVKSYYYNESNFESDELKEKFLIQFGVLLPLEAI
SHVKNKIDNIESQVYTKTNFINQKDKKGSIEDSVLNIVFVTTRTKYCSQILQLLLKYFDK
NPNKISEVYSILEANYGLVTEREYIDYTLAENTISELANLDLTKSYNQELIVTILKQFLK
IEIERTEAHEEKFTFGRYKVPDSEKLKQYHRSILKLLANLYNIGSCETRFYIEKMLYDYR
RKILTYSESHRNTIFGDLRNIRKLFFNDIKNLSMIGEKIVYALHKAEVKENLPIVFDDYI
ISDRQKIYNNLTNPNHAWFYDASEIKLQQIANSYSNVWLKIFNFANQFKHSLFMNDNNIE
LVLFNMFLLSKNDKKIKFLNYMFKSNYHFVNMNPISFLENIEESSMQSVIVSSPESEKYE
WQLAYLTQLENVKNEDLQTLKSILEANSLPCYFTILNFERLILKDPSLKELLIQKAGNTN
FVISDFIREEEVPKLINLIGVKELKFWYLINLENCQNHSYNLFQKLGEKDVDFSVEVLKK
IDELRIGHSNLGYMVLHSISEFRDKKEIYKKFIRFAINRPYYYYNNMIDDIIKNDSQIIL
EILEETNNEQSAIRLVNLGVEFLENNNQKLILFNLLRAKGFGKKSFQEIHFTPYSHFYTG
SHVPVLELEKELLERIKKIFETGIDYINLLLYLNKLIDCKRKAIERELEKEF
>orf01822
(SEQ ID NO: 883)
MNESLDDITHKQFTSNLTTKADNVSVQLFFSIKGCCHITNQGRTYTWNFIYSVVDTNTSS
TDTYLKISLAASYSFPYFFTKDWVESPCMVICTKVNDFISF
>orf01824
(SEQ ID NO: 884)
MAISQMKRISLLFSKSSLDDVLKTIQELESVQFRDLKVQDNWSEALEKDEVVFPTIQISH
TSNSNHGVIEGNDALIYLMNQQQYLEATVEKLQEYLPKENTFKLVRQPPITTSYKELEKL
VKLMLPRVFLKK
>orf01826
(SEQ ID NO: 885)
MNRACIIQPSLVEIQIWLLNHVCKCSDFLSHRMRSLLDRKLDLISLLIKLFPKKNWKN
>orf01828
(SEQ ID NO: 886)
LNGGEFLETEFGHSVLAIQSVVWFSFFCLKSNASSLAHGI
>orf01829
(SEQ ID NO: 887)
LLAGILELENWGKTTELRPTLLSGPVQNKIEALKRAKI
>orf01834
(SEQ ID NO: 888)
MRSQNHNCRPSTRKVGCIGPIFFGHLLNHRKFSYQVLTITLMEEVSLDCLPSGHHVSCQQ
GSNRYIGDRTCSNSFLIRQFFRQDTTAVAST
>orf01861
(SEQ ID NO: 889)
LRQNRCYNCFHDHSCSWKSSRITSLHGCLVRFVGFDIHTHKRFIKSRNGFHDPTNNDGLP
ISHTTFKTT
>orf01862
(SEQ ID NO: 890)
LAAFTITSLKAKTKFHPFKGIDRDNSLSQSCIQFSIPLDIGTKTNWNASDDCLHNPTDGI
TTTFDLVNIVLDFLFSFLVDNRNFRLGSSLLNFSDCQIFRNIYFLTTKDHDMVGNLHIQL
SQEAFGYCTNCHPHGGFTS
>orf01863
(SEQ ID NO: 891)
MTWARMSNFPLAFKAVFNVLRRHDVQPFLVVLIDDIHSNRRPCRLPVANARSKDNLVTLN
LHTTTTTVATLTASKVLIDILSCQWKSSWNSLNNSC
>orf01865
(SEQ ID NO: 892)
MHDLAITGSRFDGMANSVAKIEVKTNTIVQLIFNHHLALHLTRMFNQGLCMFQNTLNRTI
QSRQESPQFWILNQAILDNFTHPFNQLSFSEGFNNKWINQNPIWLGKGPHHIFSKWCVNA
CLSTDRRINLSCQTSRNLNKVNTPHIGRGYKASQVPNNATTKSNDSIATSQTLLD
>orf01867
(SEQ ID NO: 893)
MVICHNDYLLRLPEFSQPLTSLGHTTFFNLNIIRMMRNIDSDFHRRVSLSLLVFFC
>orf01872
(SEQ ID NO: 894)
LIEGHLVFADKPAQALVLLRKVGSPKKVSFLTLHLYFLILKIDILKITGF
>orf01882
(SEQ ID NO: 895)
MSTTTKFNRVVTDSHDTNFLAVFLTKEGHSSHFFSSVNIRFHCLNFKSFPDFFVDLLFNR
TQFFSSYRLGSG
>orf01887
(SEQ ID NO: 896)
VTWIHSHFNPAVVRIFIVWIVGHVKFFSREIKPFRACQKLISPSDSFVTEVIPDREVPQH
FKHGMVTRSLPYVFDVVGTDSLLGIGNTWIFRDNGPVKVFLKRLLPQS
>orf01906
(SEQ ID NO: 897)
MNGHFLLLFCLFNIFFHLVNIELSKQVLTVLDWETLVQXXIPFIN
>orf01911
(SEQ ID NO: 898)
MRLRDLRRVDFPDPDGPIKAVISLGWKDRETLFKAFFLL
>orf01914
(SEQ ID NO: 899)
LKNHSNVFTHFINVDFWTVDINSTIENLPSYFSNINSIIHAIETA
>orf01915
(SEQ ID NO: 900)
LHINPLNGFIFTIVNMDILSRKGYFFFRKGKDMLLIPVIC
>orf01920
(SEQ ID NO: 901)
LSPFQHCHSSGSIFFNLHFLNRNILQSFDNPFLGLIRENKIEKFCSQLIGLPQCIHMLIR
PQGPIIATYIFWT
>orf01921
(SEQ ID NO: 902)
MNXXNSRCNHPTWSNFLDILEVDFLGNIVGQKIRSHDLKNPVQVFTVIDMTIHIQVVKTN
MVILADRLFQGFILRSTDKFFIKIRLVRSHNLRFNNMDFSTVAVHENKGRHHVDELLPRF
IINSKATVAKKSIVAQGFRFDGNFFRKTRQTNHLNIIFCDNPDQIIVFQNGLITNSQFNR
LHP
>orf01930
(SEQ ID NO: 903)
MWYFYNTDGSMATGWVQVNGSWYYLNSNGSMKVNQWFQVGGKWYYVNTSGELAVNTSIDG
YRVNDNGEWVR
>orf01931
(SEQ ID NO: 904)
LTFIKSWAIEIFCFDWNFLDKNLGLGSFFNNSCLRVFFLT
>orf01932
(SEQ ID NO: 905)
LLLSCRKVIVCFIFSSKWNKNFFNLAFSWNFDNCIRGFFSINSNLFGNITSLWINIVGPC
RSYIAILSINCNRIFTTVFCFIFFITNSRT
>orf01949
(SEQ ID NO: 906)
MRFIVGRFTSFSLGIEFSPTSKLDDLLFKIAFLMILATWIKARKTKGAT
>orf01961
(SEQ ID NO: 907)
LVNCEPLEAYRQLEEAELVGCWAHVRRKFFEATPKQADKSSLGAKGLAYRDQLFALERDW
EALPADERLQKRPAPNGRLLCLVPPSVSFSRFKTRKGN
>orf01963
(SEQ ID NO: 908)
LKRNKIWKKTLTYPVEREEITYKRKKAKGKRQAILAQFDSEEVHHRLENCICPDCQGELK
EIGASLQRQELVFILAQLKRVNHIQHAYKCQTCSKNNPSDKIVKAPIPKAPLAHSLGSAS
IIAHTIHQKFILKVPNYR
>orf01964
(SEQ ID NO: 909)
LKIIQQQSATIDSLTNELALLREQVAYLTQKLYGKSSEKSVCPSGQLSLFEEEQNMEEDS
DLPS
>orf01972
(SEQ ID NO: 910)
LICQTIKYWHKFHLHIGRCKLLIGLIPILNFFIRADIDCLLVLLSLIDRQNGKQFNLCQW
IIASNGLNDSFEIIESLIHRNILSDIICPNQKKNFIYCSTI
>orf01978
(SEQ ID NO: 911)
MSFSCSDSCFSILLLDGDIHENTTFSPLSILFISHRFNSLIGNEVPHLIDNELLISIFFH
RFRWFNNVRMPSKDNIRSPIDHLVIKSFLFFSWFQSILNTHLKHDNGDICFLLCPFNFSL
HLIFV
>orf01981
(SEQ ID NO: 912)
VVEQIPVGHNSGSFFLFLLLRLLLSPLLRNSISFLTSQGIPWKLSNNKTKPIDKSTASKS
IATNPLLLHLR
>orf01988
(SEQ ID NO: 913)
MLKLIIYQFQYSKRQWLGTIPLLFVSSLIVGTSLFGIASSIKTANINASQLFQMLIIFGG
TTLFFLISNNIRLLIDIFKKDYQLWTILGASRTQLSLLVSGQFYLMAVIVSSIGTILSFI
MADSYYKFLQNLLGRDELPDLVITANIQSILLSIFIVPTIVGIGAYFYSSRILKISSILK
PKKKKRKVTVTGFVNISVRLFLWLLCIGSIVSAGFIRNKEIIEKQSSIILFLLIIHILII
QSLSPSIQMFLIKFLMRIFPTENYVINTGFWNLLSNPSYLKSIQTSMSMGVTLISGFILY
TQNMYSFMNTANGVNEARASFIAYMSAPIILIITSSISLTILSSNKDIEDIKQLKTLGVS
RLQLFKIRIGEAIIHSVLILLVSVIFNLIILILVSIIGQFLGRSLVDISGFWQPSLIVIS
LLVIFYSITKGFYLFQDR
>orf01989
(SEQ ID NO: 914)
MVNNVAVKVSNLSKEFLLGQDKTVSILKDISLSVNYGEFVSILGVSGSGKSTLLSCLSSL
SEPTSGEVVINGVNPYTLKEGKLAKFRRQDIAIIFQNYNLVPALPVLENVTLPLRLSGKS
VDSNKVKKMLDSLNFKAELSSLVATLSGGEQQKVAITRAIIADSKIIFADEPTGALDSVS
RKLIFETLRNLASQGKCVLMVTHDIELASKTDRALILKDGKISRQIIKPSADELYQALES
SKD
>orf01994
(SEQ ID NO: 915)
LALVRKFIDYFFGVLVPFPDLYVFKSCFKFAGSFTDFDTFDWWLDWCRSCSENRFFV
>orf02006
(SEQ ID NO: 916)
MPTILLLKKFYERLITNFFRLKFLFCKEILATNIFNHPLFEPDIRVITIKII
>orf02009
(SEQ ID NO: 917)
MXXGAFGQGELLLQQSRNSSITEIVSDSWAGAGRRILPLPKSVTPLVSS
>orf02013
(SEQ ID NO: 918)
MLLLISLTQLIIFLFFERFNLLLKTFLLVDLKSNKSA
>orf02022
(SEQ ID NO: 919)
MAGKKGFLFLNCHICMVTTTTCFLKERVESELLIFFYISPNRCLITVYSVLNL
>orf02029
(SEQ ID NO: 920)
VIPRYVTKHQGWDHNPHTITNSDDDPATLVTFRTFKFNVGNCTIPKNDQNGSSQKFSGIL
QCPCEIHLLDSP
>orf02034
(SEQ ID NO: 921)
LLVRKFNIQTFFIQVFILNDFGYTVNGLIVYRLLLTSSILSFNDYSIGSFRTVIVI
>orf02040
(SEQ ID NO: 922)
MRLSIQLIHDLNTHTTHSTAKMLYNVKAIKNDFSIRE
>orf02048
(SEQ ID NO: 923)
MTAQLPSDALQMALWRRKRPRNVIVHTDRGGQYCSADYQAQLKRHNLRGSMSAKGCCYDN
ACVESFFHSLKVECIHGEHFISREIMRATVFNYIECDYNRWRRHSWCGGLSPEQFENKNL
A
>orf02093
(SEQ ID NO: 924)
MEIVLVSFSISFQHFIIAYCLDFSSAGFRNSQNFSNFC
>orf02105
(SEQ ID NO: 925)
LTEKIQEHELIKTNQAEKSVQDVLDNCIERVQNNSLKSDRVTSFETPFALLFIFATIAVM
LTYGGYRVSAGYISVGTLVSFLIYLFQLLNPISNIANFVTVYSRSKGSSVALENLLAVPK
EKFEGGKSVSGRGLNFNHVYFGYDENRPVLKDITCSIFKGQKIAFVGPSGSGKSTIVRLL
EQFYKPLSGDILMEQSSIYDFNLKEWRSKIAWVSQNNAVLSGSIRDNLCLGLNRLVTDDE
LMKVLDLVSLGDEIRSMKEGLDTEVGERGRFLSGGQSQRLQIARTYLKDAEILIFDESTA
NLDADSEYAIISILYSALKEKTVVIIAHRLSTVKDVDCIFFLEERKITGSGTHKELLENH
ERYARFVQEQMIE
>orf02106
(SEQ ID NO: 926)
MKLKLLRVDTKVIMGSFLLVLSSLLALLLPLILKGLIDGSSIENIGSKVFQSFLIFIGQA
LFSSIGYYLFSQSGEKKIAKIRKKVIEGLIYAEKSFFDKSQSGELTSAIVNDTSVIREFL
ITTFPNIILSLVMVLGSIVVLFSLDWNLSLLLFITLPCMMFIILPLSNISEKYSRRLQEE
I
>orf02115
(SEQ ID NO: 927)
MXXALIPRVSIGSVGLLLLTENYVKGDLKAASRLVQDSLTLLFMFLLPATVGVVMVGEPL
YTVFYGKPDSLALGLFVFAVLQSIILGLYMVLSPMLQAMFRNRKAVLYFIYGSIAKLVLQ
LPTIALFHSYGPLISTTIALIIPNVLMYRDICKVTGVKRKVILKRTILISLLTLVKVSVN
RNHPVAVRIFLPTKWTFVELPLCSSCRCHGGWTLYGYESAYLFIR
>orf02125
(SEQ ID NO: 928)
MRERVRLSGSLFTSLKTREHIKSTMELFHKYVFFLIQEIKIKMINFLKIGDLPTL
>orf02134
(SEQ ID NO: 929)
MANHLYIVPIQVNHKSSIVNRMLTSITRNPIVSPTCLYASLITSLNFFLIFC
>orf02137
(SEQ ID NO: 930)
VIVFLSRNKDGNAFCHLDLISIANPVWGWDDDFITWIDHSHKEGIERIFGSRSDCHLI
>orf02140
(SEQ ID NO: 931)
LSNQFYFSLQTKPILKVKQFLLFQSQMTRVSEILQFSNKL
>orf02141
(SEQ ID NO: 932)
MSKKVLFIVGSVRQGSFNHQMALEAEKTLAGKAEVSYLDYSDLPLFSQDLEVPTHPAVAA
AREAVLVEDAIWIFSHSLQLLYPRYSEKLA
>orf02144
(SEQ ID NO: 933)
MEDKEMGFYLMVASMLLGLLALKIGFSQFKEKKDKFLSILTSLAGTALVLVAVWLGWPK
>orf02166
(SEQ ID NO: 934)
MLDSDIGCSRKNLLGLFWIRRRRNIHIVDRAMEKGISNRAPNKISLKACFFNFF
>orf02193
(SEQ ID NO: 935)
LLHPFTRNITCDRHILTLLGNLVNFIHIDNATLCTFDVKVSNLQEFEEYIFHILTHITSL
RQSCRIRNSKRYIQALSQGLGKESFP
>orf02194
(SEQ ID NO: 936)
VEIDAFVVVINRHCQGTLGTILTNYIVVQDMEEFNWFWHLRQVCQDFLNQFFSNDFLS
>orf02198
(SEQ ID NO: 937)
MEXXXTELAGRGFLVWHPKMDEYMEALDGHLDEISERLITLGGSPFSTLTEFLQNSEIEE
EAGEYRNVEESLERVLAIYRYLITLFQKALDVTDEEGDDVTNDIFVGAKAELEKTVWMLA
AELGQAPGL
>orf02199
(SEQ ID NO: 938)
LITRLLHRVHVLVDDFNPSICITWSSLALIYIFPVLNIIIDRVRQVHIVFLYKSHGLFSV
ILSIGLIFSIGIEIDTIRNSQNG
>orf02200
(SEQ ID NO: 939)
MXXTGSLSANFAGSTTASSSSEQNQSSNKTQTSAEVQTNAAAHWDGDYYVKDDGSKAQSE
WIFDNYYKAWFYINSDGRYSQNEWHGNYYLKSGGYMAQNEWIYDSNYKSWFYLKSDGAYA
HQEWQLIGNKWYYFKKWGYMAKSQWQGSYFLNGQGAMMQNEWLYDPAYSAYFYLKSDGTY
ANQEWQKVGGKWYYLKKWGYMARNEWQGNYYLTGSGAMATDEVIMDGARYIFAASGELKE
KKDLNVGWVHRDGKRYFFNNREEQVGTEHAKKIIDISEHNGRINDWKKGIDEKRVDGVIC
RLGYSGKEDKEWRIH
>orf02202
(SEQ ID NO: 940)
MHKNFVVVVTNFFTAVQFIQFNKEGTTCHNTTKFFNHLDSCLNSSTCRQKVIYNKNTLTW
LNGIRVHSQGIDTVLFFIVSRNNFAWQFTWLTNRRKTNSQLKGNWTTHDKSTSFRSHDHV
DFLVSSILNDFTNSVAISISISHQRTNITEGNAFLWIIFNCCNVIF
>orf02209
(SEQ ID NO: 941)
MNRCNSRQAIWKIISTLNRENTHIMLNRQVCFCFVNHISPLNVVIWENLSLEELLYAICI
CFITHKIAKQTSLTIDNAGIAMNNIR
>orf02214
(SEQ ID NO: 942)
LDSRFFCTDFFKGRQAKGCSFSCTSLSLTDNILAFKGQRNSLFLDRTSFYKTSFFNFC
>orf02225
(SEQ ID NO: 943)
MGRKPRTRPEERTELERLQAENEYLRAENAILKKLRELRLKEEKEKEERQLFKN
>orf02246
(SEQ ID NO: 944)
MLSKEEYIEEIGLIEKQNYVEVELYPLVADIINPTLKNSLSKRYVFGRRKSNMGQIYYGL
SNFPDIVILDKNYQNKARKSIEIEEWKKLRGCVEIKSLKHDLITEEKIKSTISNSFEHIT
GEMGQLIGDLLWYKKVIYTNGIEWRFLSLDDKEEIDNTIVQVVNKRIETEEAGNSFDWWK
NIKDLSFNYTDIYLSKDCIQEWDEFVKKVKEIEW
>orf02248
(SEQ ID NO: 945)
LEVCIHHHHQISCRILQACIKGCFFAKISRERNIMDCRILLPIGL
>orf02255
(SEQ ID NO: 946)
VDRTDEVSSKHCFEVVDRTDEVSNHTHGKATLTWFELDFRRV
>orf02263
(SEQ ID NO: 947)
MTPIKDKVRRVKTPMMVNPDTDLTISSVQQDYFSLALIGFSLLTGDFLSFSKGDQKTGLS
AFIKICHLIKIARLDNKITKQQEYWLYDLLMMSQGEKIQNIKQLKQVTSDILLNTPDFSS
YFEKYNFKEEAENIKSYLLAKSMDKSGRLFPSNEFGEFVSPVSFQHGFGGVLFFMNKYYV
EEDENTVKEWLTKLENYEAANFLHGYSLLFGKAGFLFGILDRYEKTKERYLIDISKRLVD
HLMRVYDNISNLDFALGKSGILLSLMKYCTIFDDKKLANFIKNNINDAYSLLESEDNGDI
YSNNFAHGRSGAAYVLKAYTDIFGDSRYQNHLQKFSDGISELLEEKLSSFSKLDNLGLSW
CDGVSGLILYLCLIDKERYSEIIYKSQLEMVQQYEAMGTSFCHGLSSLLQTTIYNKNQKV
EQFIKKILLTRSYRNNDRLLQFQGEDGINSYFDFGVGNLGIYWTLLGYTFPFELSKGD
>orf02264
(SEQ ID NO: 948)
MHIFLKNRAFRQLTVNEWISSFGDTIFYLAFINYVSSYAFAPLAIFLISLSETIPQVLQL
FTGVIADFQKNRISKYISILFIKVLLYSGVTLLLTSTDFSLFSVFFICSMNLISDTIGFL
AGYMLTPIYIRLINDDMTEAMGFRQSTSSIVRLIGNLSGGVFLGLFSISTLAFVNVLTFL
FAFLGSLLIRNRLKKEEEKIEVPPYVGMSSFFQHLKESMKLLMTMEDVMVLLWILSISQA
VLMMVEPVSAILLIHHPFMGLSTGQSLAILIMISLLHVILGGLLSGFLSKKISIRLNIYW
SLLMESLIVIDFLRGSFLLILLGSAGDAFSAGVLSPRLQAMIFGIIPEELMGSVQSSINV
INLLIPAVLSLALVFLATSAGLEVVAFALIILLLIAAYLVHQMKNLPNQEEV
>orf02266
(SEQ ID NO: 949)
MTVPDRLPARMRMDGRSIFQQIKNYFDTENKEYFKHPNTYDGISMHLEPNILTSMEHFDL
TGFHCECKDFQNQGVCKHWVAMDLYFRSLPQAIQERIGKASHKPSFASQLIPTLPSEELQ
DELVEEKATAPSLALHGQVEIRNHSLAWTLKLQVEQAPRAYVIKDIAHFIFLIFQKEDYF
VSQKIGTIRLSLNQFNQASQNLLLYIKKYFIDRNEHSYFNFSYGINPRDYGRYLETPVSY
LNDLVPLFQALDVFQYVTSKAEYPLIFLDDSPFIPEEEIFKVVKSNNHYEIINTAYFGFI
IQEKLWIRHNHFHIIKEEHRYFLDKLATWIYHYQENSPLIFSKENKAELMQVCNIISNYV
PISIPDELQIHDFIPTFAFSKTRNEIALNMVWSFGEKQVHSKQDLLTLPYTYQASKARKI
YHQLLSAGFKEEFHSLSKIKIVDFFLKELPRFRTLGQVQLDESLEKLLVEDPAVIDIFDD
ESFLSVQFDFSMISEDEVEKAIQALWNQESHYQTKQGKVLVFDDESLKVAQSLQDLRAKF
SDGKIKMHKSRAFSLSETFKDNEHVNFSRDFKKMAYDLTHPEEFDIKPYEVKAKLRSYQK
EGVKWLSMLDHYHFGGILADDMGLGKTLQTITLLEANLKPDQKALILAPASLLYNWKEEF
RKFVPHKQVEVAYGSKTERIKQIEKSATITITSYPSFRSDLEHYQKQSYDYLILDEAQMI
KNSQTKTAQALREFDVKTCYALSGTPIENRLEEIWSIFQIVLPGLLPSKKEFSKLSPQLV
AKLIQPFVLRRKKDEVLTELPELSEHLYSNELSSSQKTLYLAQLRRMQEMVSGASAYEIK
RHKIEILAGLTRLRQICNTPALFLEDYKGDSGKMDSLFELLDTIREKGSRPLIFSQFTSM
LDLIEQELEKKEMSHFKITGQTPSDKRQEMVNLFNQGEKDCFLISLKAGGTGLNLTGADT
VILCDLWWNPAVEMQAIGRSHRLGQTKQVDVYRLITLGTIEEKIQELQESKKELFNTVLE
GQESRSNLSVDDIKEILGVE
>orf02283
(SEQ ID NO: 950)
MMSMVDPIDQTFIVNLKIGKSQVFSQLQFSCHIVVYPSEVHIYQAFVIKLQNHILGPQVL
P
>orf02284
(SEQ ID NO: 951)
LPNRTRIDNQLPTSPVTKQLLVNMSINSNITGRMSHQAVKLLLFASMNQLSPPVLIRQMM
ANSHRQIPKLTMNLKRLIVEHFNFF
>orf02285
(SEQ ID NO: 952)
LIQQVQNPSTPCPWHENISQKPVFIHSYLPSICQNSLQGGGISMNI
>orf02308
(SEQ ID NO: 953)
MIDKVVRNLLLTFLFCKMTKIINFLTTILVKKKKMCYNVSKLREKKKGAMMWVLGFILFI
IFFYSNNSKKIKKLRE
>orf02309
(SEQ ID NO: 954)
VDRTDEVSSKHGFEVVDETDEVSNHTYGKVKLTWFEEIFEEY
>orf02314
(SEQ ID NO: 955)
MIAEFIDGLQKFHFLQNALITAIVVGIVAGAVGCFIILRGMSLMGDAISHAVLPGVALSF
ILGLDFFIGAIVFGLLAAIIITYIKGNSIIKSDTAIGITFSSFLALGIILIGVAKSSTDL
FHILFGNILAVQDTDMFITMGVGAAILLLIWIFFKQLLITSFDELLAKAMGMPVNFYHYL
LMVLLTLVSVTAMQSVGTILIVAMLITPAATAYLYANSLKSMIFLSSTFGATASVLGLFI
GYSFNVAAGSSIVLTAASFFLISFFIAPKQRYLKLKNKHLLK
>orf02336
(SEQ ID NO: 956)
MYEEPEVAPVHPTGPTPATETVDSAPGFEAPQESVTIL
>orf02363
(SEQ ID NO: 957)
MGNNGQFTFGYRHDFFQNQLAIFNALVDTFTRRTIDIKTLNTFINEVLNQGTRTLWTYFS
LLIITCVEGWNDTFVFFQI
>orf02368
(SEQ ID NO: 958)
LHEVVIPSIDEGKDCKGCKPWFHNREGYTPEGTNLTTTVDFS
>orf02369
(SEQ ID NO: 959)
LFHEEDTEWPSNQRQDNCPESIVDSHEVDDTYQWYKDNLFWKRHSSDKDSK
>orf02393
(SEQ ID NO: 960)
MSYFRNRDIDIERISMNRSVQERKCRYSIRKLSVGAVSMIVGAVVFGTSPVLAQEGASEQ
PLANETQLSGESSTLTDTEKSQPSSETELSGNKQEQERKDKQEEKIPRDYYARDLENVET
VIEKEDVETNASNGQRVDLSSELDKLKKLENATVHMEFKPDAKAPAFYNLFSVSSATKKD
EYFTMAVYNNTATLEGRGSDGQQFYGNYNDAPLKVKPGQWNSVTFTVEKPTAELPKGRVR
LYVNGVLSRTSLKSGNFIKDMPDVTHVQIGATKRANNTVWGSNLQIRNLTVYNRALTPEE
VQKRSQLFKRSDLEKKLPEGAVLTEKTDIFESGRNGKPNKDGIKSYRIPALLKTDKGTLI
AGADERRLHSSDWGDIGMVIRRSEDNGKTWGDKVVISNLRDNPEAKDPAAPSPLNIDMVL
VQDPTTKRIFSIYDMFPEGRAVFGMPKTPEKAYEKIGDKTYQILYKQGESGHYTVRENGE
VYNAQNQKTDYRVVVNPTEPGYRDKGNLYKGQELIGNIYFAHSTKNPFRVANTSYLWMSY
SDDDGKTWSAPRDITPGLRKDWMKFLGTGPGTGIVLRNGPHKGRILIPVYTTNNVSHLNG
SQSSRVIYSDDHGKTWHAGEAVNDNRQVDGQKIHSSTMNNERAQNTESTVVQLNNGDVKL
FMRGLTGDLQVATSKDGGVTWEKDIKRYPQVKDVYVQMSAIHTMHEGKEYIILSNAGGPK
RENGMVHLARVEENGELTWLKHNPIQKGEFAYNSLQELGNGEYGILYEHTEKGQNAYTLS
FRKFNWDFLSKDLISPTEAKVKRTREMGKGEMGKGVIGLEFDSEVLVNKAPTLQLANGKT
ATFLTQYDSKTLLFAVDKEDIGQEIIGIAKGSIESMHNLPVNLAGARVPGGVNGSKAAVH
EVPEFTGGVNGTEPAVHEIAEYKGSDSLVTLTTKEDYTYKAPLAQQALPETGNKESDLLA
SLGLTAFFLGLFTLGKKREQ
>orf02395
(SEQ ID NO: 961)
VADRTDEVSSKHRFEVADRTDEVSSKHRFEVADRTDEVSSKHRFEVADRTDEVSSKHRFE
VADRTDEVSNIYTARRS
>orf02399
(SEQ ID NO: 962)
MKIKEQTRKLTAGCSKHCFEVVDETDEVSSKHCFEVADRTDEVSSKHCFEVADRTDEVSN
IYTVRRR
>orf02407
(SEQ ID NO: 963)
MSCNCAFYRSQFFDVNSVSNYHSHQKELRFPNSILFTYFVKVA
>orf02428
(SEQ ID NO: 964)
VGLIKLTSYVFVCISNSFLTRHDKNDNICFFHGNFCLVLDLFHERSIDIINSSCINHAKR
TIEPLTRCINTVTCHSFDIFYNGDSLTSDPIK
>orf02430
(SEQ ID NO: 965)
LSSKSCIDRTNQETFHTLGLEGVGMKSGSLFCSVQISDKEKENSRLANGFLRYQFIQGIF
LLLTSYHNHRVGLEILPR
>orf02448
(SEQ ID NO: 966)
MKSKEQTRKLAVGCSKYSFEVADKTDEVSSKHCFEVVDRTDEVSNYIYGKAKLTWFEEIF
EEY
>orf02450
(SEQ ID NO: 967)
LSNSFFLIKFSSSKTSGKKRIVSDNIFIRNKFICHFKKE
>orf02459
(SEQ ID NO: 968)
MDYSKVAAEVIEAVGKDNLVAAAHCATRLRLVLKDEAKVNQAALDNNADVKGTFSTNGQY
QIIIGPGDVNFVYAEIIKKTGLKEVSTDDLKEIANKDKKFNPLMDLIKLLSDIFVPIIPA
LVAGGLLMALRNFLTSPDLFGPQSIEDMYPAIKGFSAMIQLMSAAPFMFLPVLVGISAAK
RFGANQFLGAAIGMIMTTPDLGGKEAFWDILGFHVTQTNYAYQVIPVLVAVWLLANLEKF
FHKKLPSAVDFTFTPLLSVMITGFLTFTVIGPVMLVVSDAITNAIVWLYNTTGAFGMGLF
GGTYSLIVMTGLHQSFPAIETQLLSAYNNNGTGFGDYIFVVASMANVAQGAATLAVYFLT
KNAKTKGLSSSAAVSAFLGITEPALFGVNLKYKFPFFCALAGSAIGAFVAGLTHVIAVSL
GAAGFIGFLSIKAGSIPMYIIAEIMSFVAAFAFTYFYGKTKAASVFADEAATATAETVTE
PTVEAPVVEETDTLQNETLVTPIVGDVVALADVNDPVFSSGAMGQGIAVKPSQGVVYAPA
DAEVSIAFPTGHAFGLKTRNGAEVLIHVGIDTVSMNGEGFEAKVAQGDKVKAGDVLGTFD
SNKIAAAGLDDTTMVIVTNTADYASVAPVATGSVAKGDAVIEVKI
>orf02466
(SEQ ID NO: 969)
MTESYTWVEADRATLSRYRHGQGHLTDQFFSFKVQRPAAKTLIASISTGKGMGPSFDGTP
VITSGNQNRINTIKNSFIMSSSSVRISLRKLTSQRNFLRNLSSLILLAAQVAKGDATACS
HQRISRVVGQDSHETLSLTEFF
>orf02467
(SEQ ID NO: 970)
MNINNEKVWFAFYLLDMQITRPTPTFNDRRIGLIGKLQELRFLAGNLLLR
>orf02468
(SEQ ID NO: 971)
LIKGYLPNHLALMDLCSKTTCTLDDFAGIAGRRNHRGFFCHIGNGVFLTVDKYLRNQRIR
QRKSSHHILTQLVCHSHTHLFILLQTSLSLRTKERLSF
>orf02474
(SEQ ID NO: 972)
LINLTGRNFSDILIKCLVKCFTNLLSNQLMLLPSTLKL
>orf02479
(SEQ ID NO: 973)
MIESENHCSASHSNRDYQSQHDNQGRTCQCFIIVPCHKKGSCSVGEITWNQRCQNGQDKD
HSRCLIKNT
>orf02486
(SEQ ID NO: 974)
MIARQLMVFFSTNQADTRITNMSIDSLIINNSKDFQSSSHASVSFILTNLVNLLIFNF
>orf02487
(SEQ ID NO: 975)
MGEPFTHFIDCIDLGINPSYTQVCDRHFTSDIPCTMTSHPIS
>orf02494
(SEQ ID NO: 976)
LSSDSHFIGIKAFVILILGKSNSIVLRIVGLYQDLTCFFSPTCSTCHLSQELEGSLRRTE
IRQIQGRIRI
>orf02495
(SEQ ID NO: 977)
MAVHSLGIHMQGQRNIAVGTSIHRPTLPTHDKARITTAIEHENHLLFFNQTVLDSL
>orf02496
(SEQ ID NO: 978)
MVTGIAVLLISHFMLFINNHDTQIFQRSKDSRSGTNNNLGIATLHLAPFIILFTIG
>orf02497
(SEQ ID NO: 979)
VKNGYLVPKTCYKTLGHLRSQGNLRYQQNSCLALIQGTLDNLQVNLGLPTSCNPLK
>orf02498
(SEQ ID NO: 980)
MVNLIPRLGLDLLLIDCLIFQTKQAFSSQTHHFSLLGKV
>orf02499
(SEQ ID NO: 981)
LGLQTKNNPLNQAIPLTKRHMNPHPNFQHSLKFLRNPVTIGLVRLHQGHIYDNLS
>orf02502
(SEQ ID NO: 982)
LGNHFCTICSTTYQAILQFIQIWWCQEDKDSIWNLFLDLKSTLNFNFKENIDSLVQGFID
IGQRSSIVVADIFCVFQHLSLTNQLFKFFTSTEEIVNTVHFSRTLCACRHRYRILKLVFR
TLKNLSSNRSFSNP
>orf02527
(SEQ ID NO: 983)
VGCSYICHELVTNHDHFLFVIVEFLHSTVNTKCEGLQGPVNVINPKFLNCSLNAFFGVI
>orf02528
(SEQ ID NO: 984)
LLHLWRSIRVVPSNGGIIQIDQNSLDSLRLQAWDCQIIDCFHSKIWYIIFNRHSGSFC
>orf02530
(SEQ ID NO: 985)
MIGTFAAALVAVLASFIVPIEITLNSANTEIAPPDGIGQVLSNLLLKLVDSPVNALLTAN
YIGILSWAVIFGIAMREASKNSKELLKTIADVTSKIVEWIINLIPFGILGLVFKTISDKG
VGSLANYGILLVLLVTIMLFVAPMVNPLIAFFFMRRNPYPLVWNCLRVSGVTAFFTRSSA
ANIPVNMKLCHDLGLNPDTYSVSIPLGSTINMAGVAITINLLTLVTVNTLGIPVDFATAF
VLSVVAAISACGASGIAGGSLLLIPVACSLFGISNDIAIQIVGVGFVIGVIQDSCETALN
SSTDVLFTAVAEYAATRKK
>orf02537
(SEQ ID NO: 986)
MWNKNRQLRKVKKILNQINRRKEEMALLTDEELAAKTQEFKRRLTAGETLDDILVEAFAV
VREADKRILGMFPYDVQVMGGIVIHQGNVAEMNTGEGKTLTATLPIYLNALSGQGVILVT
TNSYLAKRDAEEMGKVYEFLGLTIRLPFADDEEEKITPKEKKEIYSADIVYTTNSGLGFD
YLIDNLASSEEQKYMPEFNFVLVDEIDSVLLDSAQTPLVISGSPRVQSNFYGIIDTLMIT
LVDGEDYIFKEEKKEVWLTNKGAKIAEKFLGIDNLYAEENNVLARHLVFALRAHTLFKRD
KDYIIRKGEKDQELVLLDQGTGRLMEMTKLQGGLHQAIEAKEHVKLSPETRAMASITYQS
LFKMFNKISGMTGTGKVAEKEFIETYNMSVVRIPTNRPRQRIDYPDNLYITLPEKVYASL
EYIKQYHAKGNPLLVFVGSVEMSQLYSSLLFREGIAHNVLNANNAAREAQIISESGQMGA
VTVATSMAGRGTDIKLGKGVAELGGLIVIGTERMESQRIDLQIRGRSGRQGDPGMSKFFV
SLEDDVIKKFGPSWVHKKYKDYQVQDMTQPEVLKGRKYRKLVEKAQHASDSAGRSARRQT
LEYAESMNIQRDIVYKERNRLIDGSRDLEDVVVDIIERYTEEVAADHYASRELLFHFIVT
NISFHVKEVPDYIDVTDKTAVRSFMKQVIDKELSEKKELLNQHDLYEQFLRLSLLKAIDD
NWVEQVDYLQQLSMAIGGQSASQKNPIVEYYQEAYAGFEAMKEQIHADMVRNLLMGLVEV
TPKGEIVTHFP
>orf02538
(SEQ ID NO: 987)
VSNKLHILQIGNRNWSHYYEIPENIEWHFFWPGSTTAIKKVMKMEGIRTFSGVVIENPDY
LPDLLPLINILTPYTIFYSDICASYSPLVEEFLKKTCAQVTDFSNPRELLRILSKALFKG
QYGDKLTPIDMVVNPYFAGSIRYNGYENLELVGSYGEDFRPLISWKYNIRASEWNPIELW
LEYEKDLSCDIRIVVRNIQDGSTADFIKERIFTTDDMEAAILLDDDFSSFISVSLEAKGN
GRLKIGALHQRLTRYQFGKFVLGGNIIRSKNREEINYFFYPGDFKPPLNVYFSGYRRAEG
FEGFGMMKSLGSPFLLFQDPRIDGGAFYLGDDDFENAVRRVIQHHLDLLGFSNKELILSG
ISMGTYGALYYSSDFEPKAVIVSKPLTNLGLIAERGRLEAPGLFPTAFDILRHHSQGKAD
IDSINILNARFWERFRGADFNQTIFGLSYMKEEDYDPIAYDSLVDSLYSTGARIMVKGTS
GRHNDDNDSTILWFVNFYKMVLEQEFGRKY
>orf02539
(SEQ ID NO: 988)
MYYFIPFLESMNQSWQVDIVPWYQTTHRLEFDDVLHQIRIFKREGIKSKIVLLPYHPHMR
YLLHRQDLLEVEAFSVFDAIQDIENEEIYPLQLKDLAWDEDCDFIYTPFLIAVKQKGELH
AHIEFGTEGFISYITYFKDNQVDFICYFDDRGFLSSLVKYQDNQAVSRYYYNSNAEWQIK
EYLQGIHTKVEVNPRFSHRFRKSTYQSMDEVVWEFFEKFLTAEYKEGESFVLAAQTKYQN
QLLKHLPEHADKILTFFIERNQEDDLNLHHQAVKQAKILISDRQDFLERLHQHYPQFTYK
MHHLPSFDTRLKLGVSQRVKESKLYVQLDLNTPLNSEALYEVLNFVSQNPLTEIVFATFN
AEGYQIEALQKHLFTLISERLNFRDLLKESIISGAENKLEENKEENYRFQIVNLNDEIGL
IRELEYTRLIVDLNPIANIYTQIAGISAGIPQINLSESEYVTHLQNGYILSDLSEFSKAG
HYFLDTLEHWNQALIHSIDKIRQNTGNQFVQKWERWLEEAKSEQ
>orf02540
(SEQ ID NO: 989)
LKKKLKSVVVKRVMWTICFIFVYILGSRLTLPFVNVNDTSFLGGNAAFLAFSTAMTGGNL
RSLSLFSVGLSPWMSAMILWQMFSMSKKLGLGNLPLEIQERRKMILTFIISFIQTLAITL
NLPIQEGVNHDLVLILNILLLISGTFFLVWLSDLNSLLGVGGSVVILMSSMIVSVPENIV
RSIIDLHVNLLFIISLLLISIAFLYIAVRVQKARYRILVNKIMIHNRFKRYSYFDIMLNP
AGGMPFMYAISLVSIPQYLLMLLHIFVPKTRWVDNWIAEFTIGRPVWVYTYIIVLFLLGI
AFAFVNMNGEQIADKMKKSGEYIYDIYPGEDTALYINRLVLRFAVIGSIYILLMAGIPML
IILYEPRYMQLSMLPGLFLMFNGMIFNVKEEINALTLNESYRPLVERK
>orf02541
(SEQ ID NO: 990)
MKINITNIYGMSGQSTALIAQNETVKIAKKLDFHELSFYFYNIYSDSEGELNSRLDGVLA
KLGYGDIVVYQSPTWNGREYDQAFIRKCKILNTRIITFIHDVPPLMFPSNYYLMSEYIEM
YNQSDLVVVPSEKMKERLIQEGLTVQKIIIQGMWDHVHNYPLKQPSFQKKLSFAGSVERF
GHLSNWSYSTPLDIFSESNYENSNPRVSFKGWKTDPELLFALSEGGFGLVWGTNENPADE
EDYYRLNISHKVSTYLAAGIPVVVPSYLSNASFIKEKGLGYVVDSLEEANRLVEETTVEV
YQQMVENVSKVSYALKEGYFTKKLLTDAIMQLLDI
>orf02543
(SEQ ID NO: 991)
MKAIVLAGDKNYLTPILTTIKSILYYNQNVKIYILHQDIPSDWLQELKIQVEKLGSVVEG
IYIGDAIDSEWKTQAHISPIAYARYLISRLITEDRVVYLDSDIIVNGDLSPLFELSLGDY
SLAAVRDVDGNGFNSGMLVIDCQKWREKDVTSMLFDKTVEYMSYLDHTDTDGFNGDQTIF
NLVFQNHWLELDKRFNFQVGHDIIAFYSHWDSHFELDEEPLIIHYTTYRKPWTTLMGYCY
RDLWWSFHDVTFDQISDHYQGRFAVKRVYDFHDINLFTFTDSQDLLYIDELAQSLLDIAF
HIGAYTDMGDILLALDKYPNVYLYPSMVGAVIDEMIEKSDAYLDIHKGSSMDFIVNRYTS
AGRPVLTFDVTNKNQLEEIVVPSQSPLEMIKVIKKLKSDKMETKAIVFGANYQYADKVLT
TIKSICCHNRGLRFYLINSDFPTEWFYNLNRKLTKLDCEIVNARVNSSHISQYKTNIHYA
VFLRYFISDFVEEAKVLYLDCDLVVTRDLSPLFDLELGDYPLAAVKDLGGQIYFGEHIFN
SGVMLINNRLWKQEEVRKQLIEMTNELHDKVAQSDQSILNLLFKDRWLALDFKYNCITLH
THFSDYRPESGTYPPIIHYLTERKPWGLYECSIYRDVWWYYNAQDWSDMSQVTPSLTKDQ
VSQYTGVQYSALVYTFSSDLRNMGYLIENLPDVKFYVAAPVMVADSITDLLAYPNVSVLS
DIAGQPPLIDSLVEGCDFLLDINADIEVDGIIERFRQAGKPVFAFESVVHGEQGQFLYDQ
AHPEEMVLAIEAYCQNGELPVKKFQSYPKVLDIQQSLDYILEHHTSVIRYGDGEMDIMMG
HGIPYQDYDETLAEQLRSMIQLESSPELLVCLSDVFEGLERYNSESVNFWKKHLEHYKDA
YQQYCTASFYGSTFISRPYMDLKDKTASVAHFEKLKQLWDERDILIVEGENSRSGVGNDL
FDNAQSVERIICPSRNAYSKVQVIQEAVEKYADGKLVFLMLGPTAKVLAYHLSQKGIQAI
DLGHVDSEYEWFKMGATSKVKFSHKHTAEHNFDQEIQFVEDEIYNKQVVVRI
>orf02545
(SEQ ID NO: 992)
MTDKASKKAIVLGADSNYMDKVETTIKSVCSHNRDIRFYIFNSDFPTEWFQLMNKRLSVL
NSEIINIKITDDTISHFHLPTPHLSSATYLRYFIPNFVFEKKVLYLDSDIVVTSSLTALF
DIDLDGYPLGVVPDIPTTDEEFNSGVLLIDTNRWREEDIYRQLFELTIAHHEHVYGDQGI
FNILFKDRWKRLDITYNLQVGVDAHRYYMGDYDWYELFEGVPCIIHYTTENKPWKHFRFN
RFRDVWWFYYGLNWNDILLRTHVLKETFLELISPISKHVSIFTNTGDIESIGYLLEKLPD
VQFHIVAPTYFSPNVIELQRYSNCYIYPCADPKMKQDIIDKTDICLDINYGPAMDQMLQE
MVRRGKTIYSFDCTNHFFNGESTVFTVDEIDELIRSVKELKM
>orf02546
(SEQ ID NO: 993)
LKDLVSVVIPVYNVENYLEECIQSVLNQTYTNLDIVLVNDGSTDASAEICARFAEIDGRV
RVFHTENRGAALSKNFGVTQALGEYVLFVDSDDIAEKRMVETLYRQVEETGADIVIGNYF
LYDENDGQYKLYVLERDFCIEELSAQELIDRQAGKWHLNASAFIMPVFKLFKKDLLLQVP
LLMVGALMMKQRFIDCS
>orf02550
(SEQ ID NO: 994)
MRGGVDTTQVMTETVEDKVSHSITGLDILKGIAAVGAVISGTVATQTKVFTNESAVLEKT
VEKTDALATNGTVVLGTISTSNSASSTSLSASESASTSASESASTSASTSASTSASESAS
TSASTSISASSTVVGSQTAAATEATAKKVEEDRKKPASDYVASVTNVNLQSYAKRRKRSV
DSIEQLLASIKNAAVFSGNTIVNGAPAINASLNIAKSETKVYTGTGKDSFYNIPIYYQLT
VINDGSKLTFTYTVTYVNPKTKTLGNISKKMSNGYSIYNTGTSIQTMLTLGSGLGKPSGV
KNSITDKNGKQVRPYNTSTMTMWRSGYTWANGAQMNGFFAKKGYGLTSSWTVPITGTDTS
FTFTPYAAKTDRIGTNYFNGTRKVVESSTTSQSLSQSKSLSVSASQSASASASTSASASA
STSTSASTLASTSASTSASTSASTSASTSASTSASTSASTSASTSASTSASTSASTSAST
SASTSASTSASTSASTSASTSASTSASTSASTSASTSASTSASTSASTSASTSASTSAST
SASTSASTSASTSASTSASTSASTSASTSASTSASTSASTSASTSASTSASTSASTSAST
SASTSASTSASTSASTSASTSASTSASTSASTSASTSASTSASTSASTSASTSASTSAST
SASTSASTSASTSASTSASTSASTSASTSASTSTSTSASTSASTSASTSASTSASTSAST
SASTSASTSASTSASTSASTSASTSASTSASTSASTSTSTSASTSASTSASTSASTSAST
SASTSASTSASTSASTSASTSASTSASTSASTSASTSASTSASTSASTSASTSASTSAST
SASTSASTSASTSASTSASTSASTSASTSASTSASASASTSASTSASASASTSASTSASA
SASTSASTSASTSASTSASTSASTSASTSASASASTSASTSASTSASTSASTSASTSAST
SASTSASTSASTSASTSASTSASTSASTSASTSASTSASTSASTSASASTSASASASTSA
SASESASTSASASASTSASVSTSESTSTSGSTNSRPQETITNSRQELPNTGEKQSVKSGL
LGLILGLTGLGLVAKRRKRDDED
>orf02551
(SEQ ID NO: 995)
LKAFVEAHPDAFLREIAARFDCALPSVWAVLKQIKVILKKTTSFKEQKPEKVSEFLDILD
NLKDLPVLYIDETGINRYLYRPYAGAPRGEKVYDKISGRRFERTNEVEQKLNGSFLIRYI
DSQIRE
>orf02552
(SEQ ID NO: 996)
MAYSTDFKQGALDSIKEGHRHVEAAKVFDVGVRTLFTWEKKDVNKGT
>orf02559
(SEQ ID NO: 997)
MKIKEQTRKLAAGCSKQCFEVVDETDEVSLKHCFEVADRTDEVSNHTYDKVKLTWFEEIF
EEYHTKKPCSSR
>orf02561
(SEQ ID NO: 998)
MKKSDVLDLIKYHYEGRETEFRNQSIAIARNFNKHGDTQIAQYIMGLMSQSDRFMPQIEN
PSEYLTPAKLDIGPLPLPLSIMNDLKGIINAVNHHIGINKFLFVGSPGTGKTESVKQVAR
LIGKELLVVDFSHLVDSKLGQTVKNLATLFNEINNLPFKQNYIILFDEIDSIVLDRVNQN
DLREMGRVTSAFLKELDRLSPEIVLIATTNLFENLDKAVTRRFDAIIDFDRYTDEDKVEV
ATIILNELLKQFKNVARDLKLFKKIINSANVIPNPGDLRNSIRTSLAFSDPSDPHDYQKR
LLRSLHNGRNLSISKLSKLGFTVREIEILTGISKSSVSRELSED
>orf02597
(SEQ ID NO: 999)
MRFIVGIFISFSPEIEFLSSLKFLTDFVEICLLWQVMTP
>orf02598
(SEQ ID NO: 1000)
VVEIIYFLIIIIASGLGSISGMGGGIIIKPLMDSFGYHSVSDIAFYSSFSVFIMAIISTT
KRFSQSKEIKWRLIFTVSFSSVLGGFLGHLIFQVLLSQLSVRLVSIVQMILLFVMLLVSF
VLTDFKKTYQFDKIGFYMICGLLLGLISSFLGIGGGPLNVSLLMVFFSISIKEATMYSLA
IIFFSQLSHLATIVVVTGLNQYHLAPVPVIFLASICGGVLGTVVSKVLPENWVRYCFKGM
LFFVMGMTLYNLFHIL
>orf02599
(SEQ ID NO: 1001)
MMGTNSEEGFLDDFEGPQVAVSVKDFSIADTPVTNQEFAQFVKETGYKTLAERQEWSFVF
ILFVPEAEREGYPHPAGAPWWLQVSNACWKHPYGENSNLVGLEDHPVVYVALEDALAFCN
WSGMSLPTEAQWEYAARGGRQSEYPWGDTLLEGGYYHANTWQGRFPYENTALDGFIGTAP
VYEFLPNDFGLYQMIGNVWEWCRNPRYTLLASFNEDDYELPKYGIQDEEYAIRGGSFLCH
CSYCNRYRVAARNGCISTSTSSHLGFRCLKE
>orf02602
(SEQ ID NO: 1002)
MVQTKQPNIILIVVDQMRADALSLNSKDKLVSTPTLDMMASVGYNFENAYSPVPSCVPAR
AALLTGLDQDKSGRVGYQDEVPWNFTNTLPKVFKDMGYQTECIGKMHVFPSRQRLGFDHV
LLHDGYLHVDRKYDKAYGSQFDYASDYLAFLKGKVGYDVDLIDDGMDCNSWEARPWDKDE
KLHPTNWVVSESISFLQRRDPTVPFFLKMSFEKPHAPLNPPKYYFDIYMERLPQFLDLHI
GNWEVLEKQIPSIYALRGKLKEDDQRRMVAAYFGLITHIDHQISRFLTALKEFRHDKDTI
IWFVSDHGDQLGEHYLFRKGYPYQGSIHIPSFIYDPAGLIAGNRGTIKQLVKIQDIFPSL
VDLAGGTTTDELDGRSVKNLLFGQYEGWRTEFHGEHALGKDSSQYILTDQWKFIWFPVLN
HYQLFDMKKDPHEMNDLYPSEKYQPIVRQMKKKLVDFLRYREEGFVVDEELVPVELSKIT
PTLTKTGDSQS
>orf02604
(SEQ ID NO: 1003)
MNTMLDKMQEKLSPIAMKVENQKFLVALRDSFVGTMPVIMTGSIALLLNAFLVDLPQQFH
LESITKTFQWLVDINNLVFKGSIPIVSLLFIYCLGVNIAKIYKVDTVSAGLVSLASFVIS
IGSTVTKSFPLANVGDVKLDQILQGIDNLAFDGKNLMVTIGNVIPGNHINARGYFTAMMI
GFLASIIFCKVMKKNWVIKLPDSVPPAIAKPFTSIIPGFMAMYIVAILTYVFHLLSNDLL
IDWVYKVLQTPLLGLSQSFFAVILMIFLNKLFWFFGLHGGNVLAPIMEGLFGVAMLANLD
AFQKGEPIPYIWTSGSFGAFVWFGGLGLVLAILIFSRNSHYRKVAKLGLAPVLFNIGEPV
NYGLPVVLNPLLFIPFVLSPVFMATVAYWATSWGLVSPVTQNVTWVMPPILYGFFSTAFD
WRAIILSVVCLIISVLTYFPFVKMADKTELS
>orf02605
(SEQ ID NO: 1004)
MNESNLESVMGLIMYGGEAKSNAMEAIQAAKKGDFSKANRRLADANAALLQAHKAQTEML
TREAQGEKTSISLLMVHAQDHLMTSLTFVDLAKEVVEVYERFEKN
>orf02606
(SEQ ID NO: 1005)
MAKVTIMLACAAGMSTSLLVTKMQKAAEDKGLDAEIFAVPAPEAEEIVATKEVNVLLLGP
QVRYLLGDFQEKLKDRQIPVAVIPMTDYGMMNGSKVLDLAESLLD
>orf02607
(SEQ ID NO: 1006)
MKRLISANPSEILQMNAEELKQSILASEGRVVLSENVVTRETFVGDITNSEIARAFGADM
ILLNCVDVFEPKIYALDSSGDDVIHRLHQLVACPIGVNLEPIDPSAKMLEETQEIVAGRV
ASVETLNRIEELGFDFVCLTGNPGTGVSNREIIKAVQTAKENFSGLIIAGKMHGAGVNEP
VAELSVAEQLLEAGADVILVPAVGTVPAFHDQELREVVDLVHSKGGLVLSAIGTSQETSD
TDTIKEIALRNKICGVDIQHIGDAGYGGLATVDNIYALSKAIRGVRHTVSRLARSVNR
>orf02608
(SEQ ID NO: 1007)
MEKLLQEKLLPVAARLGNNKALVSIRDGITLTIPLLLIGSLLMVIASFPIPGWEKYLGDI
GVADYLWKGVDSSFGLLGLVASFGIAYFMARQYKVDGIPAGIVSLSSFITVTPFIRGEAG
AGMPTAFMASKGLFVAMILGLINGYIYQWFINHNIQIKMPDGVPPAVSKSFSAIIPGAVT
IVGWLIVYATLDKLSLPNLHEIAQVALGGPLGLLGNNVIGLLILIFLNSSFWFVGLHGGN
VVNAVMKPLWLANLDANKVAYQTGETLPNIFTSVFMDNFVFIGGGGATIGLVLALGYLAH
KKKASKQLKTLAPITVIPGLFNINEPAMFGVPIVLNILLLVPFILAPMFNLLVAWGAMAS
GLVPLTYTDPGWTMPPVISGLLATGSISGSLLQIVLIVLDVLLYLPFVIAIEKRFKLLED
>orf02609
(SEQ ID NO: 1008)
MTLSKKQLQLRAKILETVYTLGPISRIEIATKTGITPATTSSITNDLIKENILLELGEDE
HDTSVGRKKILLDIQAKRFYYIGCELSEKHFTFALGDNLGNILKEEKEIVTKQLIQEKGN
QLINQTLKQFLNNCSDYEIEAIGIALPGRYLDDYKITTNNPLWQHIDLEMIQSHFDKPLF
FSNNVNCMAIGKRLFSRQQNDPNFAYFHFARGMHCSYIYDGNIYGKGNLMIGEIGHTVVS
SEGEECSCGRKGCLQTFAGESWLIKKSKILYHQSPYSLLPSLVKNADDIDIQVILTAYQL
GDTGIITLIHQALLYLSQTILNISMMIDSQKIYLHSPLLTNQHIIQKLYSEMNYKPKLLY
NRLPEVIIEPYNDFTAAHSAIALCLYHTILHS
>orf02628
(SEQ ID NO: 1009)
MPFKENLICQHRNHHCSVFFISLGLLHNIHIEIDISQTRASFLDLSDYLQAVLMILQKFC
QAIGLAQRLDLLQLHLLHLTRLLL
>orf02636
(SEQ ID NO: 1010)
MYLLLLVVKDHIALIDKEMHVWRPNCILRDLTNFFIKRNHIVTHKTNGSTTKR
>orf02637
(SEQ ID NO: 1011)
VLTLMNHFIKEIQGISINHLTILIKNSIFKLNLKNRIIG
>orf02641
(SEQ ID NO: 1012)
MKIKEQTRKLAAGCSKPCFEVVDRTDEVSSKYCFEVVDRTDEVSSKHCFEVADRTDEVSN
HTYGKATLTRFEEIFEEYKGVPR
>orf02655
(SEQ ID NO: 1013)
VCQRMDARTCKTTIIAVHNVLTALQQTWIAVQLYQTK
>orf02656
(SEQ ID NO: 1014)
LHLGKSILSLPVKGKDLEFLVHLFVINHWIGSPSRTSTFCRCKVLNGME
>orf02657
(SEQ ID NO: 1015)
LEQTVIIANNPCELYWDNHLSFLSDSLLKQVIVHLKRICLDIHHDRGCSHVRNDTT
>orf02673
(SEQ ID NO: 1016)
MLEEGTKDQLAELTYPFGRGVNLSFGIKDVPKLYQKVMEANYPIYRLLTKRKFRVSDPYI
YPHKFAVLDPDGYFLRFSE
>orf02689
(SEQ ID NO: 1017)
MIACRHDICKSQKGLEHPFCIIRRLTRDFNQRPVCIVEANIFCLKITPQIITNMIVARTV
KSSKTGITLTTSMCKRDNHKITWFHRRNGFPSFFNNPNRFVSTIFMSSFRFWITVPP
>orf02705
(SEQ ID NO: 1018)
MNMNKDQIAILNGADNLNLTLWITLKEICKEGCKSFFPVRNTCRMLDIGIPYRLGLSLSN
SSVLNGMDV
>orf02725
(SEQ ID NO: 1019)
MHKLRIFVNQLCRRFGIILGPFLVLGFQVLTQELELAIFFDLREEVLLQVIPQVCHFCYL
RKEFTTLNQHELTSHDHVLTRHFQTHGLQG
>orf02732
(SEQ ID NO: 1020)
MQVTIEADSGLFLLSPVVQLLKVEIDVKQVTMSLDNLGRTKLSHQTFWVAGVEVHVPFNN
ADALPKWRIRT
>orf02734
(SEQ ID NO: 1021)
MKNGIDFAHIAITDFFHNQAIAMGIAHYNDGLLCHDGNTSKSFLTAKAR
>orf02758
(SEQ ID NO: 1022)
MSIVKSHSFSISLGIFNSFWNNIHTSECFYFLCKGKSNRSNSTISVNQMVFFINIQRFYC
FAIEDFCLLRI
>orf02759
(SEQ ID NO: 1023)
LNTLLPPDNLCLFTIYLTGFSCICINSYCHNFWEIFNQLFYQLS
>orf02778
(SEQ ID NO: 1024)
MYNKVIMIGRLTSTPELHKTNNDKSVARATIAVNRRYKDQNGEREADFVNMVLWGRLAET
LASYATKGSLISVDGELRTRRFEKNGQMNYVTEVLVTGFQLLESRAQRAMRENNAGLKGQ
IWXXIHSLIN
>orf02782
(SEQ ID NO: 1025)
MQFTRTTHHPKTLFTTKFAWENEIPFWHHGTRKSHNGFQPNTRIRCSCNDLHYLVTCDCN
LADVEVVTVWMSYHLDNFTNNKLRFLIINNFFCKTFRL
>orf02784
(SEQ ID NO: 1026)
LTALCNFKQARNLKNVPSYCFPIFFEENTGYLAFAFHIQFGISAIFHDDHKDITDMFFVI
RKNHCFLFLLFQSC
>orf02788
(SEQ ID NO: 1027)
MKIKDQTRKLAAGCSKHCFEVVDRTDEVSSKHCFEVVDEADVV
>orf02789
(SEQ ID NO: 1028)
MYYSVDDVVSNAFKKRMILDSFFAFNCSGTMKVSTWVYDKGEWYYVSSSGSMIANDWVKD
NGK
>orf02793
(SEQ ID NO: 1029)
LTWILTKIARKDSLQLFELEANLISFLLVMSVDLAPFCFKEENF
>orf02803
(SEQ ID NO: 1030)
MEDIDEDELLIFEKVLGQLQANIKGIGGENKEISQKN
>orf02804
(SEQ ID NO: 1031)
LHLRTCFVRQTNKLSPLINRTRLQFHQTILHYTLNQITSNRLGNIEFLLDIFNQDQVLVF
LAIIQKTHNLTLRPTHKFNAATFGFLLHHQVNLMTKTLKD
>orf02821
(SEQ ID NO: 1032)
MLEIWKYRPFVSEFWNDFKNNHDKQFVDPISLYLTLKDDDDPRIEEESEALENMILQYLG
EDDAS
>orf02823
(SEQ ID NO: 1033)
MQDLLFHFYSYRLNLTFFFFELLICLLNSEFDLSKFIFVYFDEYFHEDSLKMNLHQFSFS
F
>orf02829
(SEQ ID NO: 1034)
MAFNQFNRCIGLSIPTAPNVPGTIINRSYLHDATVPNNVREKT
>orf02845
(SEQ ID NO: 1035)
LTDFHDFKFIFFENLFKSRQLYLQSQNSVLSNLWLAT
>orf02850
(SEQ ID NO: 1036)
MKKLFILISNLLASLFFVWVFTIWTDTYVSHYYPNVVVHDSSPETTFQHVATRLEKLAEE
TDSFIAIQHQDPNSEGTTVFSYTTFGDGKLPDGLQEKNLEDAQSSSVETNYFVFDGHLDI
HLLREELSQLGLTNMHLTIPSKLSTLMAIFSNGFQLISLLIFILTFVALTLISQISQLRS
SGIRLISGEKRWSIFLRPVGEDLKAIAVGFSLAGVLAILMQKILSLPTQSLMTIGAGLLS
YNLILLSISLFFAQLFAVGIKKIHLMQIIKGQVPVRGIISLILIGQLLAIIIVTLGIGSS
LKYSQAWQQHRIGQEIWSQERQLITLSISREGTSPGFDEQAQRKLRTWYQLMDLAVSEQK
AFLSRHQLIDRTLQNGMASSKNLITSTEWHDYNPNGNVLIVTPQYLERQNIPVDTTIEQK
MNHLNVGEFVLLLPEHLRSEEEHYKSVFEDDLTSRMSSQDERQQMTATVGYLESGQDRFV
YNTTPISYQQFLKDPIIIVITPQSTGPQSILFWIDAVQNYVLFNQLSDAQELIQRQGIEN
WVSEMQTGYHNYITLLDNIQRERWVMLAGAVLGIATSILLFNTMNRLYFEEFRRAIFIKR
IAGLRFLEIHRTYLFAQLGVFLLGFVASVFLQVEIGVAFLVLLLFTGLSLLQLHVQMQKE
NKMSMLVLKGG
>orf02859
(SEQ ID NO: 1037)
VLKWCILRINHHISRKVDNFLEGTRAHIKGQAHTAWNPLEVPDVRYRSFQFDMSHTLTTN
FRTRYFNPTAVTNNSSVTNAFVLTTSTFPVFCRTKDHFIKESFTFWFQGTIINCFRFFDF
SIRP
>orf02869
(SEQ ID NO: 1038)
MPWKELCHKLAPKVFKVIRIYSRENKKSPSNWAFCSFET
>orf02877
(SEQ ID NO: 1039)
VDSLFLSLGEESNQEINLQESFSSTDCNPTLISPETTVAQGLCQDIIYRPFT
>orf02880
(SEQ ID NO: 1040)
VNPKSLGSFFLQDSKGFKELVLGHAKLSLPRIVHNVCPQFKNASRIITTRDDFWNACYSL
QMFNIFKGIQVNGRTQFTCIGVFLVWRVVGREHNLRTQKVQFMAHQKLYITRAVHTTTFF
LENFQNSWSWSSLNCKIFLKALVPRKSLVDGSCLLTNPLLIIQVKGSRELGNNRF
>orf02884
(SEQ ID NO: 1041)
MDNLCFHNAWTDWASILKQAVVTEDDMTKQNDFFLGIIDAEFHNCLGNFAINESDMSKKI
TSHCVLCLVWPRQLDDLS
>orf02885
(SEQ ID NO: 1042)
MQHNPRIEQALIELRINFANSVCQTHHGRRMIGQARFKGMVVGLGSWIGVEFLIILGVEI
SDNPLPDRIFNFENHLRHVVTNFLDINW
>orf02886
(SEQ ID NO: 1043)
LIDLRGIVINFSASFHVDNLTCGKGLNVMRLGIPELPINLATIILEGKG
>orf02899
(SEQ ID NO: 1044)
MDALVLQKNQETIQQIAVKIRFLDGHDYYSLIDIDNRRTNQTVFPFVNF
>orf02900
(SEQ ID NO: 1045)
MAFFTEIPTRACLINLAITLHIVETCQGFNDLSLHLRVLAL
>orf02904
(SEQ ID NO: 1046)
MLLPLPFNTSKIKQIAMHSDLNQKEMIGHIFHDEDIF
>orf02909
(SEQ ID NO: 1047)
MKQTVKKLALVASIAATLGGGVSVASAAVQYPEGGVWTYGSGNGGAYSNYYHPSKYHSST
VVSRKTGSSDKGYAGAGGTSRAWIRTSWGEKVAFYYNV
>orf02919
(SEQ ID NO: 1048)
MNQENLFLLQEIKDKLLIIIDTVHIAVDFWEDIEPRLGFNGRQTWNILNGIIDEISLLVD
SSTRKKQFID
>orf02920
(SEQ ID NO: 1049)
LLGQNVRAKAHIGQHIEPFDIALNMSLRARQDHPTHTETCYAVGF
>orf02921
(SEQ ID NO: 1050)
MSVHYHAVIDFIRKDNQIVLTGNLNNLQQEFLRIKGSSWVIWIDKDDCLGIGSDF
>orf02923
(SEQ ID NO: 1051)
VTYNRIRQTSHPLCNQKCQQQGDAYNPDSLVNKNSFDITILITNCLHDTYFLGTLHDIDV
NNDTNHNRCYHNSL
>orf02924
(SEQ ID NO: 1052)
LIRQHETVAVLHVIFIIDYTYNLRLKLSNLTSFGSTFYNAG
>orf02954
(SEQ ID NO: 1053)
LHTSFRSSVGHSHTWHQDIVRPILFSRFNDSIVILWQNCPTFN
>orf02968
(SEQ ID NO: 1054)
LRLAKLVPSLKIALKSFSLFTRKFFFKTQLHLLNYYYYTIFFKKANHSKVLDNFIRKRSW
KNNFPNSLY
>orf02973
(SEQ ID NO: 1055)
LKAEQQAIKNIQFLEQDLPKNPLEKEFDCLAVSRVLHHMPDLDADLSLFHQHLKEDGKLI
IADFTKTEANHHGFDLAELENKLIEHGFSSVHSQILYSAEDLFQGNHSEFFLTVSQKSLA
>orf02974
(SEQ ID NO: 1056)
MKHDFNHKAETFDSPKNIFLANLVCQAVEKQIDILSDKVILDFGGGTGLLALPLAKQAKS
VTLVDISEKMLE
>orf02978
(SEQ ID NO: 1057)
MVDLQSFFTRKYLNLNSVDAYLILPRLQGHLSYPQDFFLLQDFCFLLPIFLNLSQKEGRN
AGKDS
>orf02991
(SEQ ID NO: 1058)
VTENPAPFVFTVSINSFFTVVAFTTGTDARNQDLVTFFEVGNSFPNFFNNPNPFVAKNGT
TLASRNIPFDNMEVCSTNSGFYNTHNSICWLANNWFVYINKRSKSWFNIRXXIPFIN
>orf02993
(SEQ ID NO: 1059)
MRIRNSPFDHILQTIFEFEDRTCQVTCRFEACSSICNDNWEFSQHIISVFQSPSCHTVCD
KSDVFCSFLFDKNFASLWIYVVTITDQLCIGMWQLVHGSNHTQFTVSQPTHSIVSMHPNT
RSSIDCFFGFIKSRV
>orf02994
(SEQ ID NO: 1060)
MSKSNRHTFARNCTNKVFHPITFWCKGNFIKQAICRFLPRMKLLNTRVSHISWILCPLKS
FCEIWTFIINPTNLSTCCFFIMVSKIFSDCKQLLISGC
>orf02996
(SEQ ID NO: 1061)
MQCTFNVVVHHIYTCISMNMSIHKSWGNAITCIVNHLSPFRNLLYMFPKLAVHKFQVTTS
TNSVWVEKLIRFNIVRHNVNLLKRLILQFIMSITL
>orf03011
(SEQ ID NO: 1062)
MTLHQTFRFQNLEMPCQSSLINFQTLLNRHLVTRRMLQQKQ
>orf03023
(SEQ ID NO: 1063)
LLSSFQDAVKFFAVVFFRKVQPSQEVAPNASSFTDQFMGG
>orf03025
(SEQ ID NO: 1064)
MVVTTFNDFSIFKDNNLICIENGFQAVGNDETSSTCYNHLHGMLNLAFRHRIYV
>orf03031
(SEQ ID NO: 1065)
MAEFNSVITTVTGIGDRLGAVILAEIRNIHAFDNPAQLQAFAGLDSSIYQSDQIDLAGRM
VKRSSPHLR
>orf03041
(SEQ ID NO: 1066)
MQQYVDIKKQYPDAFLLFRMGDFYELFYEDAVNAAQILEISLTSRNKNADNPIPMAGVPY
HSAQQYIDVLIEQGYKVAIAEQMEDPKQAVGVVKREVVQVITPGTVVDSSKPDSQNNFLV
SIDREGNQFGLAYMDLVTGDFYVTGLLDFTLVCGEIRNLKAREVVLGYDLSEEEEQILSR
QMNLVLSYEKESFEDLHLLDLRLATVEQTASSKLLQYVHRTQMRELNHLKPVIRYEIKDF
LQMDYATKASLDLVENARSGKKQGSLFWLLDETKTAMGMRLLRSWIHRPLIDKERIVQRQ
EVVQVFLDHFFERSDLTDSLKGVYDIERLASRVSFGKTNPKDLLQLATTLSSVPRIRAIL
EGMEQPTLAYLIAQLDAIPELESLISAAIAPEAPHVITDGGIIRTGFDETLDKYRCVLRE
GTSWIAEIEAKERENSGISTLKIDYNKKDGYYFHVTNSQLGNVPAHFFRKATLKNSERFG
TEELARIEGDMLEAREKSANLEYEIFMRIREEVGKYIQRLQALAQGIATVDVLQSLAVVA
ETQHLIRPEFGDDSQIDIRKGRHAVVEKVMGAQTYIPNTIQMAEDTSIQLVTGPNMSGKS
TYMRQLAMTAVMAQLGSYVPAESAHLPIFDAIFTRIGAADDLVSGQSTFMVEMMEANNAI
SHATKNSLILFDELGRGTATYDGMALAQSIIEYIHEHIGAKTLFATHYHELTSLESSLQH
LVNVHVATLEQDGQVTFLHKIEPGPADKSYGIHVAKIAGLPADLLARADKILTQLENQGT
ESPPPMRQTSAVTEQISLFDRAEEHPILAELAKLDVYNMTPMQVMNVLVELKQKL
>orf03051
(SEQ ID NO: 1067)
LTNLSSVDSEELFQFYRERGNAENFIKERKAGFFGDKTDSSTMIKNEVRMMMGCLAYNLY
LFLKQLAGDEVKALTIKRFRRLFLHIAGKYVSTARRHILKFSSLYAYSKQFQALFDTICQ
INLILPVPYRARGQGKTCLTE
>orf03061
(SEQ ID NO: 1068)
LFDDRQAINICPPTNGSLRLTSLQVDQNPCPPSTNLNKILARSQFLNHIQQISLSLELLQ
ANLWNLV
>orf03092
(SEQ ID NO: 1069)
MKIKVQTRKLAAGCSKHCFEVVDRTDEVSSKHGFEVADRTDEVSSKHGFEVADRTDEVSN
IYTVRRR
>orf03093
(SEQ ID NO: 1070)
MDFFNYLLWMICHNHGLHTLLLSKDCVCHTARDKDGNHRIKSVFPTKGQTCYQHDSSIYQ
ERNTTDILTRFLTNSQADDVRPTTGDIVSKSKTNPQTHNNTPKKGIDNGILCQGCHRDKL
DKEGTHRYRDKGKDGELMANLIPS
>orf03104
(SEQ ID NO: 1071)
MIKQIKAHLNKSIQSIIGQKVEFVKQDEQAFTRKRRLSLETMIRTILGMGGKSLSKELLD
ARLTVSNSALVQRRYQIKPEAFYALFKEFTAPIPLNTDFPIFAADGSDICIPRNPMDTET
SIQTQTDVKSYNLIHINALYDLTTGVYRDVSIQDKHAQHERLALIQMMEASPFRESSCYH
G
>orf03108
(SEQ ID NO: 1072)
MAHFQEKGWLYIIRIRDGKQSMPSSFNLPNTECFDQKVSLKLSRKQTNQLKKLYRDFPND
YHFIPHNSIFDFLPETSRKQDPVTLYELPFRMVRLKVEEGKYETLVTNTDYSVQELKNLY
ASRWGIETSFRDLKYSIGLVNFHAKKKEGILQEIFARFTNFNFCRWVTSQVAIDSSHKKQ
RYKVCFSDAAYACRLFFNGSLSSHQLKNYLKKQLSIIRPNRKYSRKIKAQSVVDFICRVT
>orf03110
(SEQ ID NO: 1073)
MCELDILHDSLYQFCPELHLKRLNSLTLACHALLDCKTLTLTELGRNLPTKARTKHNIKR
IDRLLGNRHLHKERLAVYRWHASFICSGNTMPIVLVDWSDIREQKRLMVLRASVALHGRS
VTLYEKAFPLSEQCSKKAHDQFLADLASILPSNTTPLIVSDAGFKVPWYKSVEKLGWYWV
SRVREKVQYAP
>orf03139
(SEQ ID NO: 1074)
LSIQVETLELRVIFKEIKEIVKQFHQLHTMAFKRQVPLTIPVTM
>orf03154
(SEQ ID NO: 1075)
MGALGYYEGFVPYVSNQYKNQAEEEGKPLSDKYIFEKILGKTYAAFKKDQINERVEKLGK
LKPITINYNGKSEVIDSKEKLQELMNKAVKDEVAQIS
>orf03155
(SEQ ID NO: 1076)
MMGDGMKEFQFERKQRFSLRKYAIGACSVLLGTSLFFAGMGAQPVQDTETSSALISSHYL
DEQDLSEKLKSELQWFELENKLLNLWEH
>orf03159
(SEQ ID NO: 1077)
VNITKTSIIKAHTTKEDGIDHIFTRFNIMSIFYSTRKIFLDKLNSTNRQFLGYIISTRCY
QSFNSVSQSIHTSSSSQAFRFGKHEFRVINRDKSKAILVNHYHLNLAFFISNHIVNSDFC
RSSCRCIDSHNWQAFFSRLMKPFIILWFSTICSHDRNTTSCILWRTPAKTDDKVTAMFLQ
SSYPICDIFTSRVWLYIAKDDIFDSFCIQWF
>orf03175
(SEQ ID NO: 1078)
MSMDNCIDIITSLILNQMHIPFARWQAFSLYNISINIHHYNIGFFDFKEINTRRGNCHQL
FFTIENAEIPTCSFRQICFY
>orf03182
(SEQ ID NO: 1079)
MNIAIRIILNFFRVMGNHQNSLAMMMGAVVHEFVKFIFTSCIHPRCRLV
>orf03183
(SEQ ID NO: 1080)
MLLIMSIQTTEPAFSRIATRLDKFIDRTWKTSIKTGNLLRKIGYSQFLTLRICL
>orf03184
(SEQ ID NO: 1081)
LQNSKTSLDERRLSRSIFFSQGNKFPTINTIIDMFKNRLLIIIEGQILYRNISHYLISPT
KAVKNR
>orf03197
(SEQ ID NO: 1082)
MSNSFVKLLVSQLFANLADIFFRVTIIANIYIISKSVIATSLVPILIGISSFVASLLVPL
VTKRLALNRVLSLSQFGKTILLAILVGMFTVMQSVAPLVTYLFVVAISILDGFAAPVSYA
IVPRYATDLGKANSALSMTGEAVQLIGWGLGGLLFATIGLLPTTFIILVLYIISSFLMLF
LPNAEVEVLESETNLEILLKGWKLVARNPRLRLFVSANLLEIFSNTIWVSSIILVFVTEL
LNKTESYWGYSNTAYSIGIIISGLIAFRLSEKFLAAKWESILFPLVAMAIVTLTILYFPN
AQMFLLFSALVGMLSQLKEVPESVFLQETVEENHLVNVYSVLEVISTLAFSVFVLLMSYI
TESFGISISFWLSAICLMIEAILIYIRRDYFK
>orf03198
(SEQ ID NO: 1083)
MSKLLDKILSRENMLEAYNQVKSNKGSAGIDGMTIEEMDNYLRQNWRLTKELIKQRKYKP
QPVLKVEIPKPDGGIRQLGIPTVMDRMIQQAIVQVMSPICEPHFSDTSYDFRPNRSCEKA
IMKLLEYLNDGYEWIVDIDLEKFFDTVPQDRLMSLVHNIIEDGDTESLIRKYLHSGVIIN
GQRYKTLVGTPQGGNLSPLLSNIMLNELDKELEKRGLRFVRYADDCVITVGSEAASKRVM
YSVSRFIEKRLGLKVNMTKRVEISRFWVLEIIRWLEKPSTSR
>orf03202
(SEQ ID NO: 1084)
MFLRCATFKLADSRLNIFTCFFFGEIRFNSRNQVVKAFITDGTVISTIIVRGTVPCNQWT
KTCPAAFDIINGDVGFWKAVVDNAK
>orf03203
(SEQ ID NO: 1085)
MLQITCVVCISCTKVSLVFTWENKDHTTVTQTCVKVNWL
>orf03204
(SEQ ID NO: 1086)
LRSLIRQITYFITPRTCCINNQTGLDFKHLVCQEITSYNTCNLATFVKEEAFCLHVVGNE
GTVLVGTFDVFNHETRIVVTEVKIHSTSYQAFLLQVWLAFQDLILAQNLVRSWCVAHTC
>orf03205
(SEQ ID NO: 1087)
LHFNQTSLKTASCRLQGYTSSCDSSTDNQEVQGAFLHFFN
>orf03216
(SEQ ID NO: 1088)
LKIDHTQLSPSNLLNTFVTPFIFYLKHSINLTNAEIICFSFYFHADFLVHYPENQ
>orf03224
(SEQ ID NO: 1089)
LKKVQHTQNVDFNKKLSRIKTKYLYGLKEKSEAELTLKTKETKEELTAAFEQFKKDTLKS
GKKVAEAEKKAKAQKEEDRRNYPTNTYKTIELEIAEAEVGVAKAELELEFAQAQVQIPQD
TEKINAAKSKVEAAKSNVKKLEKIKSDIEKTYLYKLDNSTKETPKSRVRRNSPQVGDSRE
LKETIDKAKETLSTYMVTRLTKLDPSVFWFADLLMDAKKVVEEYKTKLEDASDKKSVEDL
RKEAEGKIESLIVTHQNREKENQPAPQPGGQAGGSMVVPPVTQTPPSTSQSPGQKATEAE
KKKLQDLIRQFQEALNKLDDETKTVPDGAKLTGEAGKAYNETRTYAKEVVDKSKKLLSQT
AVTMDELAMQLTKLNDAMSKLKEAKAKLVPEVKPQPENPEPKPQPEGEKPSVPDINQEKE
KAKLAIATYMSKILDDIKKHHLKKEKHHQIVALIKDLDKLKKQALSEIDNVNTKVEIENT
VHKVFADMDTVVTKFQKGLIQNTPQVPEAPKSPEVPKVSDTPKAPDTPQVPEAPKSPEVP
KVPEAPKAPDTPQVPEAPKSPEVPKVPDTPKAPDTPQVPEAPKSPEVPKVPDTPKAPDTP
QVPEAPKAPDTPQIPEAPAPETPKTGWKQENGMWYFYNTDGSMATGWLEYNGSWYYLNAN
GAMATGWLEYNGSWYYLNTNGAMETGWLEYNGSWYYLNTNGAMETGWLEYNGSWYYLNTN
GAMETGWLEYNGSWYYLNTNGAMETGWLEYNGSWYYLNTNGAMETGWLEYNGSWYYLNTN
GAMETGWLEYNGSWYYLNANGSMATGWLKDGDTWYYLEASGAMKESQWFKVSDKWYYVNG
SGALAVNTTVGGYRVNANGKWVN
>orf03230
(SEQ ID NO: 1090)
MDREILKFFQDLLSILSHNDMITLFCQKCCNSFSNHFLVICN
>orf03232
(SEQ ID NO: 1091)
MFITLRRICLRACVVEKEQSYLKFLFFQKRPVSFLHVKSVLAGI
>orf03233
(SEQ ID NO: 1092)
MVKTTNRLEAIGFSFILFENLFKPRQLYLQPQTSVLSNLRLAA
>orf03239
(SEQ ID NO: 1093)
MTRKLNPSYTNVASATTLTFNQVASTFRKACLDHVVNLTRNNLKGICQLTPLQLHDTRLI
>orf03270
(SEQ ID NO: 1094)
MRTFFLYSSAFKKHSSPSPINDGLYHLLLQSLYNILELIHDIFQSLKGFILKSTFTNLFP
HLFNGVHLWCVWRNKCKANISRNL
>orf03277
(SEQ ID NO: 1095)
LVSVFYSLLQVDNVDSVTFSKDVLSHLRIPATSLVTKVYTSLKKLFH
>orf03286
(SEQ ID NO: 1096)
LIVWILKNHTDLTTYIPNIFLSQTLAINYNLSRFCFQ
>orf03287
(SEQ ID NO: 1097)
MPYNRKPFSTFHVKRNILHIVVVLIFFITKRKIFYINY
>orf03291
(SEQ ID NO: 1098)
MFKKMSNSSRILFYISVNFCDKRIYRTKLYSDTPVNLFKFLFRQKSNCQSIGQTSSINLF
FYSWIVFFFKNNLCHSIPSIK
>orf03304
(SEQ ID NO: 1099)
LADGSGKLAEGGTKLTSGLEDLQTGLASLGQGLGNASDQLKSVSTESKNAEILSNPLNLS
KTDNDQVPVNGIAIAPYMISVALFFAAISTNMIFAKLPSGRHPESRWAWLKS
>orf03310
(SEQ ID NO: 1100)
MKNTVKLEQFVALKEKDLQKIKGGEMRLSKFFRDFILQRKK
>orf03330
(SEQ ID NO: 1101)
LVEQLTFNQWVTGSSPVRVIYAGLAELADAPDLGSGA
>orf03344
(SEQ ID NO: 1102)
MKIKEQTRKLAAGCSKHCFEVVDETDEVSNHTYGKVKLTWFEEIFE
>orf03352
(SEQ ID NO: 1103)
LIDVLFINSFIGRICFYCYRRIHATCLFLQLFSIVILNVAHTLKHSIFIVITFISRCRNF
IIVRILLENQFSRNQGIDNRVGQSRY
>orf03353
(SEQ ID NO: 1104)
MVNVNQVSIEVKNTFKNWNFTSSIELTTFSKFSQSPTMT
>orf03364
(SEQ ID NO: 1105)
MGFSMKLIHDLDMHTTHSTAKMLYNVKAIKNDFSIRE
>orf03368
(SEQ ID NO: 1106)
LKSVGSRVEDKRYQETIVATFSDILKRSREMQDQNIKXXFIH
>orf03372
(SEQ ID NO: 1107)
MLTIEPTKAPLVNVCDTANTARIDDEPYSPGDFXXHSIH
>orf03373
(SEQ ID NO: 1108)
MRWNIGCHPNRDTSCSINQKVWKTRWQDQGFPFIGIIVINEINCIFVDITKHFQSNLAHT
CLGITLSGSTISIHGTKIPMTIYKHVTVAPPLSHTDHGFINRGIPVWVIFTHDIPCNTSR
FFMGFVWGHTQFIHSVKNATVNRF
>orf03380
(SEQ ID NO: 1109)
MTDENTFLQLSEGWSLFRSDFLLCIKHFLNSFSSSKGQLKASPTRCNLDNRLVVLL
>orf03390
(SEQ ID NO: 1110)
MGGNPPMKKYSIVDKIVLSTKIKRIIIFTVFRENWEPYMKKYTEVFQSQFPNLNIDYLLL
DTEQIDLDSYLDADIIIIGGGNTEKYIATYVNQEFKNYIDHMLNKGAKVIGFSAGALLLG
EKVYVSPNDNSDHQIKIKDGLGLFSQFLISVYYDSWNDKANKDRAEELVNVPIIPLNDHS
CLVLDKLGNIIEKID
>orf03393
(SEQ ID NO: 1111)
MYGGEAKSNAMEAIQAAKKGDFSKANRRLADANAALLQAHKAQTEMLTREAQGEKTSISL
LMVHAQDHLMTSLTFVDLAKEVVEVYERFEKN
>orf03396
(SEQ ID NO: 1112)
MLARSKNCFMKSLSIFLLIFYFFDSYQISKKRRSLIGL
>orf03399
(SEQ ID NO: 1113)
VTAHRIFGTSSIHSKLIGLAMLGITAMKIICHKLNRNHINIFRRLGIQGKTEFLLIHLIR
QVKMNHLSQGMNPTICPTSTVNSNGLPFI
>orf03402
(SEQ ID NO: 1114)
MLKNGIISWKDFKSFFCQGCQTSHCYKPMQAVQGIGSQIS
>orf03403
(SEQ ID NO: 1115)
MRFLADQDRIQHHRYSWALFDKVQGLLSHADSREKTNLNSPKFHITQAI
>orf03405
(SEQ ID NO: 1116)
MSYGRPYILNVDGAIHDGWLAISNYENSLNKDYLFYILSSNVVYSQGLSLISGAVVKNLN
SDKVASILIPLPPLAEQQRIIEAIESALEKVDEYAESYNRLEQLDKEFPDKLKKSILQYA
MQGKLVEQDPNDESVEVLLEKIRAEKQKLFEEGKIKKKDLDISIVSQGDDNSYYEEVPCE
IPESWEWVRLNDITSYIQRGKSPKYSNIPIYPVIAQKCNQWSGFSIDLARFIDPETVHSY
QKERLLRDGDLMWNSTGLGTLGRLAIYHENKNPYVWAVADSHVTVIRVLSGVINCHFIYN
FLSSPIVQSVIEEKASGSTKQKELLTKTIKEYLIPLPPLPEQSRIVDRIEQFFAHIDALI
>orf03424
(SEQ ID NO: 1117)
LAQISILHFDFLSIDKHSHTVFNTLRKSLQTTLALSATSKQCFEQLAASFLVCSLIFIEY
KV
>orf03430
(SEQ ID NO: 1118)
MGFKVSHFKIPSSHLSINVLRTVENFTEIGQGLLHISP
>orf03431
(SEQ ID NO: 1119)
VGFFDFGLTNSCRQVRQFTQTVQDFLVCCHQGIVKEGQGYAGICFKFHPSLGNIGKFVIA
IVRRLRHKSIVANMAHLNVDLFQFRKGLLEILKSVKIALVITAKLVDVFASFLDCTQEIL
TVLV
>orf03439
(SEQ ID NO: 1120)
MNITYIVGNGLDLQYGLKTRYRDFYEFQNKVYISRTENEEKYSNFIYESLFSDKVNDYEN
WSDFELSIGKLTKDNDLISSSIEIKEKFIDDFSEVVDDLREYLRIQQEKNLEKGNAIDFI
STLDDMRTSLPVINQPAIDKKYNENPHQDDIVNIVTLNYTHVIDKLYNGSAKSFRNQLRA
NLYNFYIEPPIHAHGTVDVCTVLGVSDEIQISNSFDEEQKESLIKNLVLKNYRENMDVKN
SDIIKNSDIIILYGVSLGETDGYIWNQIAEQSIRSSVPVIIYHYVPHFDAGNPTRVKRLY
RNVEDKFIQNSGIDLELEKKLRDNLIVVIGKTIFNLMER
>orf03440
(SEQ ID NO: 1121)
VGAKFNDEKTKHIVTHYISRDALNKTITVLSKIIEVFEEHFDRAITCEMFSDSSTFASIN
FSEYGISKSKFQQYLRDSCFIENFGVEHTTVSDIQNSIVTFYDVHTDIFRLLNKLNIDIS
EANIMNQTTVLLDEKNIELLLSKAPYLVSMIVEDFSKLSVDDFSLDNNDLKINLPSPMNE
PVVGVIDTLFDKRVYFNEWVEYHDFVSPDISKDSQDYKHGTAVTSLIVDGANLNPNLDDG
CGNFRVRHFGVSLQSGFNSFTIIKQIKEIVSQNADIKVWNLSLGSNDEIRENFISAEGAL
LDEIQFENDVIFIIAGTNASVINGKRKRIGAPADSLNSIIVNSVDFNNQSVSYSREGIVL
SFFVKPDVSYYGGGNGDFINVCEPLGLGRVAGTSFAAPFIARKMAYLIHIMGLSREEAKA
LLIDAAIPWNDKKTFTDLSLIGNGIVPIKMDDILSTPDDEIKFIVSDISRAYDTYNYDFP
VPISSESYPYVAKATMCYFPNCSRKQGVDYTNTEMQLTFGRLKSDGIKSINKDNQHAEDT
PGYVRENAARNIFRKWDNVKHIGESFTSRKRAKAILNPSNPQWGMSIKTIERLKSGDGQG
VRFGVVVTLKELNGVNRIEDFIQQAELRGWLVNRLQVEAQVDLFNSLNEEIEFE
>orf03442
(SEQ ID NO: 1122)
MFVADIMISDYSSAPIDFLLLNRVVFLYLPDFKEYQSDKNPFFEVFKVSKTKGIALDPFD
EIIGRFQFGVRIV
>orf03450
(SEQ ID NO: 1124)
MGFSMKLIHDLNTHTTHSTAKMLYNVKAIKNDFSIRE
>orf03453
(SEQ ID NO: 1125)
MEQLHFITKLLDIKDTNTQIIDVVNRDSHKEIIAKLDYDAPSCPECGSQMKKYDFQKPSK
IPYLETTGMPTRILLRKRRFKCYHCSKMMVAETPLVKKNHQIPRIINQKIAQKLIEKISM
TDIAHQLSISTSTVIRKLNDFHFECNFRNLPKIMSWDVETVRGVTVSIGRWR
>orf03462
(SEQ ID NO: 1126)
LDPWDGNSQKPRFQGLWKFIQRDSRKWRERRFYGPTFGKHLTNKKVFDKVFELFTRPGNI
IIIFINFCGFTSEIRNRGKFFGLIEDNLKQVHPIFQTVFKTFLKDKEKIINALQLHYSNA
KPEATNNLIKLIKRNAFGFRNFENFKKRIFIALNIKKERTKFVLSRA
>orf03466
(SEQ ID NO: 1127)
MVLYFWKVFQRVPNKLWKNMGENFQVRSLQDKIIQNLTNKGFSYFDAKMPIDEWDSQVDE
ETTQELISRDLISNILSMPESMKDTN
>orf03469
(SEQ ID NO: 1128)
MSKSHSFSISLGISNSFWNNIHTSECWYFLAEGKSNRSNSTISVNQMVFFINIQRFYCFA
IEDFCLLRI
>orf03470
(SEQ ID NO: 1129)
LNTLLPPDNLCLFTIYLTGFSCICINSYCHNFWEIFNQLFYQLS
>orf03475
(SEQ ID NO: 1130)
MKLLSIAISSYNAAAYLHYCVESLVIGGEQVGILIINDGSQDQTQEIAECLASKYPNIVR
AIYQENKCHGGAVNRGLAEASGRYFKVVDSDDWVDPRAYLKILETLQELESKGQEVDVFV
TNFVYEKEGQSRKKSMSYDSVLPVRQIFGWDQVGNFSKGQYTMMHSLIYRTDLLRASQF
>orf03476
(SEQ ID NO: 1131)
MYYLPVDFYRYLIGREDQSVNEQVMIKCIDQQLKVNRLLIDQLDLSQVSHPKMREYLLNH
IEITTVISSTLLNRSGTAEHLAKKRQLWTYIQQKNPEVFQAIRKTMLSRLTKHSVLPDRK
LSNVVYQITKSVYGFN
>orf03484
(SEQ ID NO: 1132)
MKRIQLNMNETKKYLVIKAIAQGKKTKKRACVELNLSERQINRLLLAYQQKGKEAFRHGH
GNRNRKPKHAIPDEIKERVLKKYLSYETYKPNVLHFCELLAEEEGIKLSDTTVRKILYKK
NILSPKSHRKTKKRVRKQAKLNLNQPLDNPILPTAKDFLEDPKKVHPSRPRKKFAGELIQ
MDASPHAWFGPETTNLHLAIDDASGNILGAYFDKQETLNAYYHVLEQILANHGIPLQMKT
DKRTVFTYQALQL

In some embodiments, preferred INV200 antigens are selected from the polypeptides orf00159 (SEQ ID NO: 626), orf00162 (SEQ ID NO: 628), orf00163 (SEQ ID NO: 629), orf00164 (SEQ ID NO: 630), orf00165 (SEQ ID NO: 631), orf00166 (SEQ ID NO: 632), orf00201 (SEQ ID NO: 639), orf00209 (SEQ ID NO: 645), orf01109 (SEQ ID NO: 747), orf01137 (SEQ ID NO: 751), orf01138 (SEQ ID NO: 752), orf01309 (SEQ ID NO: 783), orf01313 (SEQ ID NO: 786), orf011315 (SEQ ID NO: 787), orf01431 (SEQ ID NO: 810), orf01433 (SEQ ID NO: 812), orf01434 (SEQ ID NO: 813), orf01537 (SEQ ID NO: 824), orf01588 (SEQ ID NO: 831), orf011642 (SEQ ID NO: 842), orf01656 (SEQ ID NO: 847), orf01800 (SEQ ID NO: 875), orf01801 (SEQ ID NO: 876), orf01810 (SEQ ID NO: 879), orf01812 (SEQ ID NO: 880), orf01818 (SEQ ID NO: 882), orf01988 (SEQ ID NO: 913), orf01989 (SEQ ID NO: 914), orf02105 (SEQ ID NO: 925), orf02106 (SEQ ID NO: 926), orf02263 (SEQ ID NO: 947), orf02264 (SEQ ID NO: 948), orf02459 (SEQ ID NO: 968), orf02538 (SEQ ID NO: 987), orf02539 (SEQ ID NO: 988), orf02541 (SEQ ID NO: 990), orf02545 (SEQ ID NO: 992), orf02604 (SEQ ID NO: 1003), orf02608 (SEQ ID NO: 1007), orf02609 (SEQ ID NO: 1008), orf02850 (SEQ ID NO: 1036), orf03197 (SEQ ID NO: 1082), orf03439 (SEQ ID NO: 1120), orf03448 (SEQ ID NO: 1123), and immunogenic fragments thereof.

7. Sequences Identified from 23F
>orf00010
(SEQ ID NO: 1133)
LPVTFDFLIEGSTKGNIDKLDTATDCHNWFILTKRFLQECQFKFVTDQIVIIAFDGLFLS
IKLRMNILASCQNEFVNHFHIITYN
>orf00017
(SEQ ID NO: 1134)
MSLITHKRFISCNENIKHYKRLIDKAKKCVNDLMAEFNSVITTVTGIENRLGAVILAEIR
NIHAFDNPAQLQAFAGLDSSIYQSGQIDLAGRMVKRGSPHLR
>orf00027
(SEQ ID NO: 1135)
MQQYVDIKKQYPDAFLLFRMGDFYELFYEDAVNAAQILEISLTSRNKNADNPIPMAGVPY
HSAQQYIDVLIEQGYKVAIAEQMEDPKQAVGVVKREVVQVITPGTVVDSSKPDSQNNFLV
SIDREGNQFGLAYMDLVTGDFYVTGLLDFTLVCGEIRNLKAREVVLGYDLSEEEEQILSR
QMNLVLSYEKESFEDLHLLDLRLATVEQTASSKLLQYVHRTQMRELNHLKPVIRYEIKDF
LQMDYATKASLDLVENARSGKKQGSLFWLLDETKTAMGMRLLRSWIHRPLIDKERIVQRQ
EVVQVFLDHFFERSDLTDSLKGVYDIERLASRVSFGKTNPKDLLQLATTLSSVPRIRAIL
EGMEQPTLAYLIAQLDAIPELESLISAAIAPEAPHVITDGGIIRTGFDETLDKYRCVLRE
GTSWIAEIEAKERENSGISTLKIDYNKKDGYYFHVTNSQLGNVPAHFFRKATLKNSERFG
TEELARIEGDMLEAREKSANLEYEIFMRIREEVGKYIQRLQALAQGIATVDVLQSLAVVA
ETQHLIRPEFGDDSQIDIRKGRHAVVEKVMGAQTYIPNTIQMAEDTSIQLVTGPNMSGKS
TYMRQLAMTAVMAQLGSYVPAESAHLPIFDAIFTRIGAADDLVSGQSTFMVEMMEANNAI
SHATKNSLILFDELGRGTATYDGMALAQSIIEYIHEHIGAKTLFATHYHELTSLESSLQH
LVNVHVATLEQDGQVTFLHKIEPGPADKSYGIHVAKIAGLPADLLARADKILTQLENQGT
ESPPPMRQTSAVTEQISLFDRAEEHPILAELAKLDVYNMTPMQVMNVLVELKQKL
>orf00033
(SEQ ID NO: 1136)
MRRKYKSIALKKELANDSGKKKFHAMKAQAIVTSQGRIVSIAMI
>orf00042
(SEQ ID NO: 1137)
LTNLSSVDSEELFQFYRERGNAENFIKERKAGFFGDKTDSSTMIKNEVRMMMGCLAYNLY
LFLKQLAGDEVKALTIKRFRRLFLHIAGKYVSTARRHILKFSSLYAYSKQFQALFDTICQ
INLILPVPYRARGQGKTA
>orf00051
(SEQ ID NO: 1138)
LFDDRQAINICPPTNGSLRLTSLQVDQNPCPPSTNLNKILARSQFLNHIQQISLSLELLQ
ANLWNLV
>orf00055
(SEQ ID NO: 1139)
LSVHFCSSHRCLLVRYNDTYSTKKGLKFETFLSVFRYDFLGM
>orf00086
(SEQ ID NO: 1140)
VDRTDEVSSKHGFEVVDETDEVSSKHGFEVADRTDEVSSKHGFEVADRTDEVSSKHGFEV
ADRTDEVSSKHGFEVADRTDEVSSKHGFEVADRTDEVSSKHGFEVADRTDEVSSKHGFEV
ADRTDEVSSKHGFEVADRTDEVSSKHGFEVADRTDEVSNIYTAR
>orf00088
(SEQ ID NO: 1141)
MDFFNYLLWMICHNHGLHTLLLSKDCVCHTARDKDGNHRIKSVFPTKGQTCYQHDSSIYQ
ERNTTDILTRFLANSQADDIRPTTGDIVSKSKTNPQTHNNTPKKGIDNGILRQGCHRDKL
DKEGTHRYRDKGKDGELMANLIPS
>orf00096
(SEQ ID NO: 1142)
MKIKEQTRKLAAGCSKHCFEVVDETDEVSSKHGFEVVDETDEVSSKHGFEVVDETDEVSN
HTYGKATLTWFEEIFEEY
>orf00103
(SEQ ID NO: 1143)
LQNDKNHKLFDNYTCQKEKDVLRCKQVKRKEERSYDVGTRIYTIYDFLLF
>orf00105
(SEQ ID NO: 1144)
MKIKEQTRKLAAGCSKHCFEVMDRTDEVSSKHGFEVVDETDEVSNHTYGEVKLTWFEEIF
EEY
>orf00106
(SEQ ID NO: 1145)
LFFKDEKQALYTKPKTKSSSFRASKVSNQTIVATTRTDCQVIALNLCDKLENGVVVVVQT
THHIGIDDVIYSKIFQHLTHSIKMSLAFFIKKVQDRRRILYCHLVFFFLRVQDTKRIFLQ
ATLAILRQGLLERCQIVNQGLAVGCTALRISKSVEVQFDTLNTDFLQKMGCHSDCFHIGS
WIARAKTLNTNLVELAQAPCLWTLITEHRSHVVELAWLLHFWGEEFIFHIGTDNGRSSFW
TEGNMAVTLVIEIVHFLGYDIGRISDRAADNLVMLKNGRAHFCVVVALENFTGKALNVLP
FGRFSR
>orf00114
(SEQ ID NO: 1146)
MEQIGKVFRQLRESRNISLRQATGGQFSPSMLSRFETGQSELSVEKFLFALENISASVEE
ILFLARGFQYDTDSELRKEITDVLEPKNVAPLEDLYRREYQKHAHSHNKQKHILNAIMIK
SYMKSMDERVELTAEEGKVLHDYLFSTEIWGIYELNLFSVSSPFLSVSLFTRYVREMVRK
SDFLMEMSGNRNLFYTILLNGFLASIECEEFTNAYYFKRVIEEHFYKENETYFRIVYLWA
EGLLDSKQGRVKEGQKKMEDAVRIFEMLGCNKSAEYYRNTTEC
>orf00118
(SEQ ID NO: 1147)
MQEHYTPKGKHLTIDNRRLIERWKNENKSNREIAGLLGKAPQTIHTEVKRGTTLQQVRKG
LYKKVYSADYAQTVYQFNRKRSVKKLILTKEIREKILHYHKQKFSPEMMVNKKQVKVGIS
TIYYWFHNGHLGLTKADMLYPRKRKGVKKQASPNFKPAGKSIEERPDVINLRLENGHYEI
DTVLLTKIKNYCLLVLTDRRSRHQIIRLIPNKTAESVNQALTLLLGEHHILSITADNGSE
FKRLSEVFPEEHIYYAHAYSSWERGSNENHNRLIRRWLPKGTKKTTPKEVAFIENWINNY
PKKCLDYKSPSEFLLGG
>orf00121
(SEQ ID NO: 1148)
MKIKGQTRKLAAGCSKHCFEVVDRTDEVSNHTYGKATLT
>orf00124
(SEQ ID NO: 1149)
VVPFSDTFKDRNQVDIFTIKISRCNSSTIGENSWDIHISNSNHRSRHVLVTATDSDEGIH
VVTTHSRLDGVRDDVTRC
>orf00139
(SEQ ID NO: 1150)
MDLKFEGVDLEYKKAKNNLPESFWETYSAFANTNGGKIILGIDEKNIDTYQRVNRLPAKL
>orf00156
(SEQ ID NO: 1151)
LSIQVETLELRVIFKEIKEIVKQFHQLHTMAFKRQVPLTVPVTM
>orf00171
(SEQ ID NO: 1152)
VQKLKKAIYKAHLKDSDDFRPETSTPNLFESCLKLCPCFLSS
>orf00172
(SEQ ID NO: 1153)
MGALGYYEGFVPYVSNQYKNQAEEEGKPLSDKYIFEKILGKTYAAFKKDQINERVEKLGK
LKPITINYNGKSEVIDSKEKLQELMNKAVKDEVAQI
>orf00173
(SEQ ID NO: 1154)
MMGDGMKEFQFERKQRFSLRKYAIGACSVLLGTSLFFAGMGDQPVQDTETSSALISSHYL
DEQDLSEKLKSELQWFELENKLLNLWEH
>orf00177
(SEQ ID NO: 1155)
VNIAKTSIIKAHTTKEDGIDHTFTRFNIMSIFYSTRKIFLDKLNSTNRQFLGYIISTRCY
QSFNSVSQSIHTSSSSQAFRFGKHEFRVINRDKSKAILVNHYHLNLAFFISNHIVNGNFC
>orf00178
(SEQ ID NO: 1156)
MKPFIILWSSTICSHDRNTTSCILWRTPAKTDDKVTAMFLQSSYPICDIFTSRVWLYIAK
DDIFDSFCIQWF
>orf00194
(SEQ ID NO: 1157)
MHIPFARWQAFSLYNISINIHHYNIGFFDFKEINTRRGNCHQLFFTIENTEIPTCSFRQI
CFY
>orf00205
(SEQ ID NO: 1158)
MNIAIRIILNFFRVMGNHQNSLAMMMGAVVHEFVKFIFTSCIHPRCRLV
>orf00206
(SEQ ID NO: 1159)
MLLIMSIQTTEPAFSRIATRLDKFIDRTWKTSIKTGNLLRKIGYSQFLTLRICL
>orf00207
(SEQ ID NO: 1160)
LQNSKTSLDERRLSRSIFPSQGNKFPTINTIIDMFKNRLLIIIEGQILYRNISHYLISPT
KAVKNR
>orf00220
(SEQ ID NO: 1161)
MSNSFVKLLVSQLFANLADIFFRVTIIANIYIISKSVIATSLVPILIGISSFVASLLVPL
VTKRLALNRVLSLSQFGKTILLAILVGMFTVMQSVAPLVTYLFVVAISILDGFAAPVSYA
IVPRYATDLGKANSALSMTGEAVQLIGWGLGGLLFATIGLLPTTFIILVLYIISSFLMLF
LPNAEVEVLESETNLEILLKGWKLVARNPRLRLFVSANLLEIFSNTIWVSSIILVFVTEL
LNKTESYWGYSNTAYSIGIIISGLIAFRLSEKFLAAKWESILFPLVAMAIVTLTILYFPN
AQMFLLFSALVGMLSQLKEVPESVFLQETVEENHLVNVYSVLEVISTLAFSVFVLLMSYI
TESFGISISFWLSAICLMIEAILIYIRRDYFK
>orf00221
(SEQ ID NO: 1162)
MSLVHNIIEDGDTESLIRKYLHSGVIINGQRYKTLVGTPQGGNLSPLLSNIMLNELDKEL
EKRGLRFVRYADDCVITVGSEAASKRVMYSVSRFIEKRLGLKVNMTKTKITRPRELKYLG
FGFWKSSDGWKSRPHQDSVRRFKLKLKKLTHRKWSIDLTRRIEQLNLSIRGWISYFSLGN
MKV
>orf00222
(SEQ ID NO: 1163)
MSKLLDKILSRENMLEAYNQVKSNKGSAGIDGMTIEEMDNYLRQNWRLTKELIKQRKYKP
QPVLKVEIPKPDGGIRQLGIPTVMDRMIQQAIVQVMSPICEPHFSDTSYGFRPNRSCEKA
IMKLLEYLNDGYEWIVD
>orf00229
(SEQ ID NO: 1164)
LHFNQTSLKTASCRLQGYTSSCDSSTDNQEVQGAFLHFFN
>orf00247
(SEQ ID NO: 1165)
MFCLTFICLIRRSGYLGSYLLLCRMNHTSHKKTGNSYTSYSNTKFTN
>orf00248
(SEQ ID NO: 1166)
LPSEIKAKLDAAFEQFKKDTLPTEPGKKVAEAEKKVEEAKKKAEDQKEKDLRNYPTNTYK
TLELDIAESDVEVKKAELELVKEEAKESRDEKKINQAKAKVENKKAEATRLKNIKTDREK
AEEAKRRADAKLQEANVATSEQDKSKRRAKREVLGELATPDKKENDAKSSDSSVGEETLT
SPSLKPEKKVAEAEKKVEEAKKKAEDQKEEDRRNYPTNTYKTLELEIAESDVEVKKAELE
LVKEEAKESRDEKKINQAKAKVENKKAEATRLKNIKTDREKAEEAKRRADAKLQEANVAT
SEQDKSKRRAKREVLGELATPDKKENDAKSSDSSVGEETLTSPSLKPEKKVAEAEKKVEE
AKKKAEDQKEEDRRNYPTNTYKTLELEIAESDVEVKKAELELVKEEAKESRNEEKIKQVK
AKVESKKAEATRLENIKTDRKKAEEEEAKRRAAEEDKVKEKPAEQPQPAPAPQPEKPTEE
PENPAPAPAPKPENPAEKPKAEKPADQQAEEDYARRSEEEYNRLTQQQPPKAEKPAQPST
PKTGWKQENGMWYFYNTDGSMATGWLQNNGSWYYLNSNGAMATGWLQNNGSWYYLNANGS
MATGWLQNNGSWYYLNANGSMATGWLQYNGSWYYLNANGDMATGWLQNNGSWYYLNANGD
MATGWLQNNGSWYYLNANGDMATGWLQYNGSWYYLNANGDMETGWVKDGDTWYYLEASGA
MKASQWFKVSDKWYYVNGSGALAVNTTVDGYGVNANGEWVN
>orf00254
(SEQ ID NO: 1167)
MDREILKFFQDLLSILSHNDMITLFCQKCCNSFSNHFLVICN
>orf00261
(SEQ ID NO: 1168)
MTRKLNPSYTNVASATTLTFNQVASTFRKACLDHVVNLTRNNLKGICQLTPLQLHDTRLI
>orf00300
(SEQ ID NO: 1169)
LVSVFYSLLQVDNVDSVTFSKDVLSHLRIPATSLVTKVYTSLKKLFH
>orf00309
(SEQ ID NO: 1170)
LIVWILKNHTDLTTYIPNIFLSQTLAINYNLSGFCFQ
>orf00310
(SEQ ID NO: 1171)
MPYNRKPFSTFHVKRNILHIVVVLIFFIAKRKIFYINY
>orf00314
(SEQ ID NO: 1172)
MFKKMSNSSRILFYISVNFCDKRIYRTKLYSDTPVNLFKFLFRQKSNCQSVGQTSSINLF
FYSWIVFFFKNNLCHSIPSIK
>orf00327
(SEQ ID NO: 1173)
LADGSRKLAEGGTKLTSGLEDLQTGLASLGQGLGNASDQLKSVSTESKNAEILSNPLNLS
KTDNDQVPVNGIAIAPYMISVALFLQQYQQI
>orf00356
(SEQ ID NO: 1174)
MEMSFIAQDFDKLNIITVLESRTQAIIRNPMNTRLSSATGSSFNKIVRN
>orf00358
(SEQ ID NO: 1175)
MELAETSIVKKNHQIPCIINQKIAQKLIEKTSMTDIDHQLSISTSTVIRKINNFHFEHDF
SRLPEIMS
>orf00364
(SEQ ID NO: 1176)
MNYIDTNEMLFVETPRKVITSDELRKKNTKYLDQKEFKLFIQNLKDEALCDYRITKYIRI
AKVLFLTGMRYGELAALNYKEDIDFSKKTIHIKHTYDFRQKERTTPKTIKSDRVITAPQK
VLDIIKEQIIENATNGFDTDFIFINTLGEPITNARVICALKRHGQKIGIEKNITTHTFRH
SHISLLAELGIPLTAIMDRVGHSDSKTTLEIYSHVTQKMVSDISSKLDKIKF
>orf00365
(SEQ ID NO: 1177)
MWMEELPNGKYKFFERYKDPYTEKLKKVSVTMEKKTPQARNQAAILLQEKIKQKLGEKQH
SVSNITFEKLYEEFEENWKHGVKNSTVYASKNVKKEILKQIEGDYLVRNLIDVYYKK
>orf00367
(SEQ ID NO: 1178)
MEIDKVKADLKQVGKRVADLSQSITNEEQTKNAFIMPFFQALGYDIFNPLEFVPEFTADV
GIKKGEKVDYAIILDGEPQILIECKSITENLTKHDSQLFRYFVTTKSKFGILTNGREYKF
FTDLDEPNKMDTTPFLTIDVTDIKENQFTEIIKFHKENFDIDNIVSSASELKYLNNLKAF
LTENITTPSDSFLRYLTSEIYEGRVTQNILTTFSPIIVKGFNQFITERVNEKLSAALNTS
VETKVTTDIPKVEAEAEEIVEVTDEIITTPAELEVYTVVKMLARDVVSPERVFYRDNRSY
FNVLVDDNIKKWVLRYRSNSKKSTIEIRDKGIFPVSTPLEVANYANEILEVIKKFS
>orf00368
(SEQ ID NO: 1179)
MTLAKLCEEYQVELCLFDGSNWHNSGFYNPDTNVLAIDHNLTPEQQIQVALHELGHKDHT
RSEHQNARLRCENEADRNMIHHLVKDALENLDDPTEFDYLKFMSYYNLKTMTNEIMVKEE
YLALVN
>orf00369
(SEQ ID NO: 1180)
MYRLDIDKKALKQLKKLDTPTRKQILSWLAKNIENTTNPRQHGKALKANLAGYWRYRVEN
YRIICDIQDDKLVVLAVEIAHRRDVYK
>orf00370
(SEQ ID NO: 1181)
MTITINFTEKNSYITDYLNKHGIDTTTMDFDDFMALMEDIEDARAADQAYMEYLADPATY
TMDEVLDELGLTREDIA
>orf00371
(SEQ ID NO: 1182)
MFETFEKIKELAKKRGKALGQVEEDLGYGRNTLYKIKNSTPNAERIAEIANYFNVSTDYL
LGRTDNPAIAGSDEFAQVNGQIIDLRKAAANTMLFDGKPLNEDDIDFITSVLSAHFKSKG
ER
>orf00372
(SEQ ID NO: 1183)
MVSILKNLEQEKDHLEKVIKVVSAGGKFLRLPYQKSHARLVRI
>orf00373
(SEQ ID NO: 1184)
MPDIANGRERVIAFLKEKGIKKATLAVAYGFKRQEVTNILSGTTKGPRANSFILQVIEDY
GIE
>orf00374
(SEQ ID NO: 1185)
MRPKRYPYSGQKESTFVKADPELVEKLLRNTSFLECLQKKPINFQIDSEEFKRLSYEAIH
DTSQVTQ
>orf00377
(SEQ ID NO: 1186)
LKNREEEWQGIIAKNAILLIIAPFYFLIIVKNGVLSKIKTVTEITAYQL
>orf00378
(SEQ ID NO: 1187)
MREVIQELLDSSMSTSAISQGAGVPWTTVSDLRKGKTSMDKMALLTAEKLYEFATTDKQ
>orf00382
(SEQ ID NO: 1188)
VEEVEVAEVKNARVSLTGEKTKPMKLAEVTSINVNRTKTEMEEFNRVLGGGVVPGSLVLI
GGDPGIGKSTLLLQVSTQLSQVGTVLYVSGEESAQQIKLRAERLGDIDSEFYLYAETNMQ
SVRAEVERIQPDFLIIDSIQTIMSPEISGVQGSVSQVREVTAELMQLAKTNNIAIFIVGH
VTKEGTLAGPRMLEHMVDTVLYFEGERHHTFRILRAVKNRFGSTNEIGIFEMQSGGLVEV
LNPSQVFLEERLDGATGSSIVVTMEGTRPILAEVQALVTPTMEGNAKRTTTGLDFNRASL
IMAVLEKRAGLLLQNQDAYLKSAGGVKLDEPAIDLAVAVAIASSYKDKPTNPQECFVGEL
GLTGEIRRVNRIEQRINEAAKLGFTKIYVPQNSLTGITLPKEIQVIGVTTIQEVLKKVFA
>orf00389
(SEQ ID NO: 1189)
VNIATLQNGHILGWQIQHIANKLTSNFWIAKDFLSYQVIGWANARMTYSHISSLFIISQF
>orf00391
(SEQ ID NO: 1190)
VSITFSLTNFFKILINLTAQVSPQVIDEKILMMDLNLNNYLSTVIQLRQDVYTGIKILHR
VRHGE
>orf00392
(SEQ ID NO: 1191)
MSRYSYSLDSRKIVFEISCFKEKKASLTLFFHLFESSIMKLATQPSFSSFYSELK
>orf00396
(SEQ ID NO: 1192)
MKIKEQTRKLAAGCSKHCFEVVDRTDEVSSKHRFEVADRTDEVSSKHRFEVADRTDEVSN
IYLRQGDVDVV
>orf00408
(SEQ ID NO: 1193)
LSLLDLRGSLCLRIYLHEPLITTVSQDFTSLSDISHF
>orf00411
(SEQ ID NO: 1194)
MDFKSFIIGLVVGIFGPYMDDLIRKKFLKSSEKKTEKSVKK
>orf00434
(SEQ ID NO: 1195)
MFEKIKGINIKSGIFEDETKLELFEGNFEGTNPVQNDRASLLFGRNGSGKSTIARGINQL
KNGEIGTDRVSFIDKNNNNIVLSDTERKSIFVFNEHYVDQKVKIAQEGLDTIVILGEQVD
IDEELDRLRTQLSESQIESQDYYAEYEEYLDEKNEKSPDFWKKEMTDSLKGVGNWAERDR
EIKGNRAASPVHNNTFQNFVDLQPILDKNELEVEFNNKKARYFSIRDSAVTINNELSLPD
INFDSNELSTLLSEKIEEPELNSRDKYLLTLLSDSTKGERHLREVKDFFEDEHQKKCPFC
TQSVSEDVKVELTNGITKLLSRAVEEHQSALRGKKIDEINQDFSGYEQIDPILIQSYQNS
INALNAKFNEINSIIDKKIDNPYNIVELPNISFSQELSQAEHDIEKINQAIIKHNSEISG
IQKLKVDLLQINNELAFYEIQDAYKKFQEKTNKKAICENNYNNSSKRVKDYEKQISDLED
KKLNIDIAVDEINKSLNYIFFSKNRLAIQNQNGNYYLLSRGKSVVPSRVSVGERNALALC
YFFTEIIQQRELADAYSHEYFIVIDDPISSFDMENKVGITSYLKYCLTRFFKGNSNTRVL
LMTHDKQTIYDFDIFLKEIMESCKEEEGGQKSKYKKLELVSGKLQEFKTSTHDYTELLEI
VFGYALGNSTPTSESFVGNAMRKILEAYGSFNYKKGIAELTTDPLIVEKIDKEYRTYFEN
LMYRLVLNGESHFKDPVKTLSIDFFDTISDEERKKTARDLLVLLYLLDDLHVLKHLEGVS
NAENRLEQWKCEILE
>orf00458
(SEQ ID NO: 1196)
MIEGDRDCADIVTQLTAVKSSVERVIEMIITENLTECINQPLDDSEAQKEPLFKAIRYII
KRK
>orf00460
(SEQ ID NO: 1197)
METSISMADFYGKYQNENLELIDVREAHEFQAGHAPGAKNLPLSTLEQGYKELKPDHEYY
VICQGGVRSASTCQFLSSQGLTVTNVEGGMNAWPGQVE
>orf00462
(SEQ ID NO: 1198)
LGGKSCLLEDRLCDIAAQTTVAADDVGLFFVQFISFLLDTLSVFDTIVQN
>orf00466
(SEQ ID NO: 1199)
MKIKDQTRKLAAGCSKHCFEVVDRTDEVSSKHCFEVADRTDEVSNIYTARRR
>orf00467
(SEQ ID NO: 1200)
MKLLSIAISSYNAAAYLHYCVESLVIGGEQVGILIINDGSQDQTQEIAECLASKYPNIVR
AIYQENKGHGGAVNRGLAEASGRYFKVVDSDDWVDPRAYLKILETLQEFESKGQEVDVFV
TNFVYEKEGQSCKKSMSYDSVLPVRQIFGWDQVGNFSKGQYIMMHSLIYRTDLLRASQF
>orf00468
(SEQ ID NO: 1201)
MYYLPVDFYRYLIGREDQSVNEQVMIKCIDQQLKVNRLLVDQLDLSQVSHPKMREYLLNH
IEITTVISSTLLNRSGTAEHLAKKRQLWTYIQQKNPEVFQAIRKTMLSRLTKHSVLPDRK
LSNVVYQITKSVYGFN
>orf00476
(SEQ ID NO: 1202)
MSLQIKLKKLAKELSKLLKDSNLETVDKDVLENSQKELQKAVLFLADEKGSEHTEAEVID
NLKEVIAKLKANA
>orf00483
(SEQ ID NO: 1203)
MKIKEQTRKLAAGCSKHCFEVVDKTDEVSYIYLRQGEADAV
>orf00503
(SEQ ID NO: 1204)
MKIKEQTRKLAAGSSKHCFKVVDGTDEVSSKHCFKVVDGTDEVSSKHCFEVVDRTDEVSN
HIRQGDVDVV
>orf00509
(SEQ ID NO: 1205)
MNDDDSRCIHIERDGKTIEFGYLNISSTDRNTSHADGLVGIFNSNFSGVRVRGIAVFLNG
PDNLDTTLVGNFQTIWNFRIICIHS
>orf00510
(SEQ ID NO: 1206)
LEFNFCRSIIKNGRDNLPNTNSTSGMATRWANHNWSDDIKDRLKTK
>orf00515
(SEQ ID NO: 1207)
MSNVDKIRKIHIIVCWMYIFLSFRAIINDTEYFLLIFLAFIYSIVSLPLYSVKNKIVSIC
LVINSILLMSFPILINKFFPESFLTYIVLISVFITELIIFHLIGKDFDTKLTNEYKKISQ
FRSKVSQSPWIKYLEISSFILTIFPSILYGTVDNHVLTLIFLIKICVDTTIKFLFIRLFD
TSTLMKRRIFFLFALDVIAYLFLGYLLVIQKAGYLFSVLLLFSNFSVPFIKEKEYELFKN
SK
>orf00516
(SEQ ID NO: 1208)
MNKKKMILTSLASVAILGAGFVASSPTVVRAEDAPQVVEKSSLEKKYEEAKTKADTAKKD
YETAKKKAEDAQKKYDEDQKKTEEKAKKEKEAAKKVDDASLAVQKAYVEYRKVQESRSNY
RNRSDYNKKLAEAQVKIDEANKKLTAANNEFKTVRAVVVPEPNALAETKKKAEEAKAEK
>orf00518
(SEQ ID NO: 1209)
LEQEVATAQHQVDNLKKLLAGVDPDDTEAIEAKLKKGEAELNAKQAELAKKQTGLEKLLD
SLDPEGKTQDELDKEAAEAELNKKVESLQNKVADLEKEISNLEILLGGADSEDDTAALQN
KLAAKKAELAKKQTELEKLLDSLDPEGKTQDELDKEAAEAELDKKADELQNKVADLEKEI
SNLEILLGGADPEDDTAALQNKLATTKAELEKTQKELDAALNELGPDGDEEETPAPAPQP
EQPAPAPAPKPEQPAPAPKPEKSADQQAEEDYARRSEEEYNRLTQQQPPKAEKPAPAPAP
KPEQPAPAPKTGWKQENGMWYFYNTDGSMATGWLQNNGSWYYLNSNGAMATGWAKVNGSW
YYLNANGSMATGWVKDGDTWYYLEASGAMKASQWFKVSDKWYYVNSNGAMATGWLQYNGS
WYYLNANGAMATGWAKVNGSWYYLNANGSMATGWVKDGDTWYYLEASGAMKASQWFKVSD
KWYYVNGLGALAVNTTVDGYEVNANGEWV
>orf00519
(SEQ ID NO: 1210)
LTISFKKQFLSSSLSSLTKRVIMNTAQATFNREAHTTFNRE
>orf00525
(SEQ ID NO: 1211)
MKIKEQTRKLAVGCSKHCFEVVDRTDEVSSKHRFEVVDRTDEVSNIYTARRS
>orf00539
(SEQ ID NO: 1212)
LKKRMNRWQFLLNQSKEMVGILLLKMKEQELIEFVVNL
>orf00540
(SEQ ID NO: 1213)
LIKVIKRKAFGFRNFNNFKKRILMTLNIKKESTNFVLSRL
>orf00544
(SEQ ID NO: 1214)
MTYNEKRLTNSLERVHMEQLKNTTDLLGLKDKNIKILSVLKYQTHLVVQAKLDSPAPPCP
HCQGKMIKYDFQKASKIPLLDCQGLPTVLHLKKRRFQCKNCLKVVVSQTSIVKKNCQISN
MVRQKIAQLLLEKQSMTEIAHRLAVSTSTVIRKLREFKFETDWTKLPKVMSWDEYSFKKS
KMSFIAQDFESKSILAILDGRTHAVIRNHFQRYQREVRELVEVITMDMYSPYYRLAKQLF
PKAHIVLDRFHIVQHLSRAMNRVRIQIMNQFDRKSLEYRALKRFWNPRFFVSRLGLNQST
GLIYYTRIASSSVRNDSISPRFECT
>orf00545
(SEQ ID NO: 1215)
MGYSLKKSCTYCEQDPEKVNRFLKELNHLSYLTPIYIYETGVETYFYLEYDRALSRQLVS
LEEDIII
>orf00552
(SEQ ID NO: 1216)
MNIAVIGLGHVGLAYALLFASKYKVVAYDIDSVKINNLKKGILPSKNEELMKFFCENNLN
ITFFDTFSEIKNNIDYYIIALPTDYDEKIGSFNTYEIEQTVSKILRVKPNGKIILKSTVP
FGFSNKLKRLFDTKNIIFVPEFLREGCSIYDNLYPSRIVVGDETVEGRKIAELFLSISTH
STANIKNVMLVSPTEAEAIKLFSNTFLALRVAFFNELDSFAERRSLNAEVVIKGVCLDPR
IGNFYNNLSFGFGGYCLPKDTKQLKKEFIEINAPVIEAIDISNTNRKQFIVKQILERKPK
IVGIYKLGMKYNSDNYKESAILSIINELLIVGIKILVYEPNLNVSIDNVIFEKNFELFTK
QSDLIVANRWDRGLEAYKDKVYTRGIWIRD
>orf00554
(SEQ ID NO: 1217)
MLNLQFAETMELTEAELEIVYGGEFGNNAVIPAGAWGGFGTPWSITNFWKKNFNDRPDFD
SDRRRY
>orf00599
(SEQ ID NO: 1218)
MGLDVGSKTVGVAISDPLGFTAQGLEIIQINEEQGQFGFDRVKELVDTYKVERFVVGLPK
NMNNTSGPRVEASQAYGAKLEEFFGLPVDYQDERLTTVAAERMLIEQADISRNKRKKVID
KLAAQLILQNYLDRKF
>orf00635
(SEQ ID NO: 1219)
LNPSYSFGKKDQFALEHCFCIKLSIFARAVTLFVSCIN
>orf00656
(SEQ ID NO: 1220)
MITGTAFILIMSLSARKLPYTIRSSVASLQQIAPSIEEAAESLGSSRLNIFAKITTPMML
SDIISGAILSWVTLISELSTSILLYNVKTRTMTVAIYTEVLRGNYGVAAALSTILTVLTV
GSLLLFMKISKSNSITL
>orf00657
(SEQ ID NO: 1221)
MLIGEGYRTFPVLIYTQFISEVGGNSAFAIMAIIIALAIFLIQKHIANRYSFSMNLLHPI
EPKKTTKGKMAAIYATVYGIIFISVLPQIYLIYTSFLKTSGMVFVKGYSPNSYKLAFNRM
GSAIFNTIRIPLIALVLVVLFTTFISYLAVRKRNLFTNLIDSLSMVPYIVPGTVLGIAFI
SLVYLEVDFL
>orf00658
(SEQ ID NO: 1222)
MECKKLNIWTASSFFLFLTYLVFLVYPIVTVLKQALIHEGQFSLANFVTFFSKAYYSETL
VNSFRVSITATVTSLVVGTLLAYLFSMYDFKGKKFLQILIIIASMSAPFVGAYSWILLLG
RNEVITKFLTNALYLPAIDIYGFKGIVLVFTLQLFPLVFLYVAGTMNSIDNSLLEAAESM
GSFGFKPIVTVVLPLLVPTLLAAPCLYL
>orf00660
(SEQ ID NO: 1223)
LLSTTEFIGLSIRILSNLHEFKILVGLLNQFFFWNLLLHKTKSNVVSDSQMWENSVVLEN
QPDIAFAGFHIIDFCIIEVKFSIFDTVETCNHTKKGRFPTS
>orf00679
(SEQ ID NO: 1224)
MITIKKQEIVKLEDVLHLYQAVGWTNYTHQPEMLEQALSHSLVIYLALDGDAVVGLIRLV
GDGFSSVLVQDLIVLPIYQRQGIGSALMKEALEDYKDAYQVQLVTEETERTLGFYRSMGF
EILSTYNCIGMTWMNRKK
>orf00710
(SEQ ID NO: 1225)
VLKIRYHEQFKKDFKLAMKRGLNAELLEEVLKIWFKKKNFLLDIVIIN
>orf00714
(SEQ ID NO: 1226)
MLGSMFVGLLVGFLAGTLTNRGEHMGCFGKMFLGWIGAFIGHLLFGTWGPIIAGTAIIPA
VLGSMIVLAIFWRRGS
>orf00741
(SEQ ID NO: 1227)
MIDDIPKRVNDVIGQAGNNAKTSRPHVGIGKSHISVPFLFPYHTANRIKNQEKVIF
>orf00755
(SEQ ID NO: 1228)
VAIDKIAGITSEKDSRAHQIFRISPTCSRCFCNDELVKWVARTIFLQLTKRCCLRSGNIT
RSNSVTLDIGSTVFRRNVAGQHFQAPFSSSISANCFTSQFAHHRTNIDNLSMPFLYHRRN
NCL
>orf00756
(SEQ ID NO: 1229)
LFDLLDHGLDTVLVCHVTDISMGFDANFTISFNPFIDQILIDIVKDNSSAGFSVGFGNSK
SNSIRSAGDESNFSF
>orf00768
(SEQ ID NO: 1230)
MKSLARLLIIHVFISIFLFFALISGAVSHTVLLLLLLFLPALNKGLEKIQSNRIPVLNAA
LFFLLISFPQLLTNPVQWKFSIFLVVTIISSLAYFYNFYQVVKEVDQKQLI
>orf00769
(SEQ ID NO: 1231)
LEAASEIETEFQSWIVLVVFNHIDGLSRDTDILGELELGNAQFLAKFFHTIHLVSFLICV
VYI
>orf00774
(SEQ ID NO: 1232)
MKWTKRVIRYATKNRKSPAENRRRVGKSLSLLSVFVFAIFLVNFAVIIGTGTRFGTDLAK
EAKKVHQTTRTVPAKRGTIYDRNGVPIAEDATSYNVYAVIDENYKSATGKILYVEKTQFN
KVAEVFHKYLDMEESYVREQLSQPNLKQVSFGSKGNGITYANMMSIKKELETAEVKGIDF
TTSPNRSYPNGQFASSFIGLAQLHENEDGSKSLLGTSGMESSLNSILAGTDGIITYEKDR
VGNIVPGTELVSQQTVDGKDVYTTLSSPLQSFMETQMDAFLEKVKGKYMTATLVSAKTGE
ILATTQRPTFNADTKEGITEDFVWRDILYQSNYEPGSAMKVMTLASSIDNNTFPSGEYFN
SSEFKIADATTRDWDVNDGLTTGGMMTFLQGFAHSSNVGMSLLEQKMGDATWLDYLKRFK
FGVPTRFGLTDEYAGQLPADNIVSIAQSSFGQGISVTQTQMLRAFTAIANDGVMLEPKFI
SAIYDTNNQSVRKSQKEIVGNPVSKEAASTTRNHMILVGTDPLYGTMYNHYTGKPIITVP
GQNVAVKSGTAQIADEKNGGYLVGSTNYIFSVVTMNPAENPDFILYVTVQQPEHYSGIQL
GEFATPILERASAMKESLNLQSPAKNLDKVTTESSYAMPSIKDISPGELAEALRRNIVQP
IVVGTGTKIKETSVEEGTNLAPNQQVLLLSDKVEEIPDMYGWKKETAETFAKWLDIELEF
EGSGSVVQKQDVRTNTAIKNIKKIKLTLGD
>orf00776
(SEQ ID NO: 1233)
MVDRTDEVSSKHGFEVVDKEKLMWFEEVFEECKKILVS
>orf00783
(SEQ ID NO: 1234)
MEGVNHVDIIKVSCCSFISQVNWMMKGKIPNREGFKFSVARFDAIDLVVVHIGHTRCQFS
RTGSRSGYDNQVATGFDVVVFAHAFWGNDVIHIRRISFDWIMKIRINSVFLKLVAEGICS
GLASVLCNDNGTNKNP
>orf00784
(SEQ ID NO: 1235)
MFNVASINGNHNLNLLFQFLQELDFVVRFITRKDTSSVEIF
>orf00790
(SEQ ID NO: 1236)
LTNQDLQAGTYLVKDYREIILSQDALEKVATNLKLDMPAKTLASKVQVAVPADTRIVSIS
VKDKQPEEASRIANSLREVAAEKIVAVTRVSDVTTLEEARPATTPSSPNVRRNSLFGFLG
GAVVTVIAVLLIELLDTRVKRPEDVEDVLKIPLLGLVPDFDKIK
>orf00791
(SEQ ID NO: 1237)
MPTLEISQAKLDSVKKAEEYYNALCTNLQLSGDGLKVFSITSVKIGEGKSTISANIAWAF
ARAGYKTLLIDGDIRNSVMLGVFKARNKITGLTEFLSGTTDLSQGLCDTNIENLFVIQAG
SVSPNPTALLQSKNFTTMLETLRKYFDYIIVDTAPVGVVIDAAIITRNCDASILVTEAGE
INRRDIQKAKEQLEHTGKPFLGIVLNKFDTSVDKYGSYGNYGNYGKNKK
>orf00792
(SEQ ID NO: 1238)
MNEKILRSSLAIIQSFLVILLTYLLSAVRETEIVSTTAIALYILHYFVFYISDYGQDFFK
RRYLIELVQTLKYILFFALAIGISNFFLEDRFSISRRGMIYFLTLHALLVYVLNLFIKWY
WKRAYPNFKGSKKILLLTATSRVEKVLDRLIESNEVVGKLVAVSVLDKPDFQHDCLKVVA
EGEIVNFATHEVVDEVFINLPGEKYNIGELVSQFETMGIDVIVNLNAFDRSLARNKQIRE
MAGLNVVTFSTTFYKTSHVIAKRIIDIVGALVGLILCGLVSIVLVPLIRKDGGSAIFAQT
RIGKNGRQFTFYKFRSMCVDAEAKKRELMEQNTMQGGMFKVDDDPRITKIGCFIRKTSLD
ELPQFYNVLKGDMSLVGTRPPTVDEYEHYTPEQKRRLSFKPGITGLWQVSGRSEIKNFDE
VVKLDVAYIDGWTIWKDIEILLKTVKVVFMRDGAK
>orf00793
(SEQ ID NO: 1239)
MKKSVYIIGSKGIPAKYGGFETFVEKLTAFQQDKAIQYYVACMRENSAKSGTTEDVFEHN
GAICYNVDVPNIGPARAIAYDIAAINRAIEIAKENKDEDPIFYILACRIGPFIHGIKKKI
QEIGGTLLVNPDGHEWLRAKWSAPVRRYWKISEGLMVKHADLLVCDSKNIEKYIQEDYKQ
YQPKTTYIAYGTDTTRSVLKSSDEKVRSWFKEKNVSENEYYLVVGRFVPENNYESMIRGF
LASNSKKDFVLITNVEQNKFYNQLLAKTGFDKDPRVKFVGTVYEQELLKYIRENAFAYFH
GHEVGGTNPSLLEALASTKLNLLLDVGFNREVAEDGAIYWKNDNLHEIIETSEQKTQKEI
DEKDILSIKQVTERFSWELIVNEYEKLFLCEK
>orf00794
(SEQ ID NO: 1240)
VTIKINNLFFVCLSFFGIVLSSSQVIVNLGLSSIIQYISYFMLMLCVFLTLIKNTLNVFA
NRIIYFLIISFLFIIGINLQNLPLSRKIYLSFSMLIISSLSTLPIKLINNLSDLRRISYY
LLHSIFLSVFLGLVFKISLVTVAVEGIGFSYGFNGGLTHKNFYAITILVSYILLYVSRKY
DAKHQIDSFVLWLDLFLLLISNTRTVYIILVVFWIIINRNFINNIKKEHRLVVTATTIVI
SLLALTFFFKHIINNSESYSHRVLGVVNFFKYYESDRFHLFFGDAELAFGNTTKGYGHNI
RSVLGWDGTVEMPLLSVMIKNGYVGLVGYIIVLFKFISSIISVKNSTKKNIGLSIFIPLL
LSATVENYIVNISFVFMPVCFCILCSIKNIKLVNNRK
>orf00796
(SEQ ID NO: 1241)
MEKLVSIILPVYNVEQYIKNCLESIQQQTYSNLEVIIVNDGSTDKSVEYCEQICKIDSRF
SITHKENGGLSDARNVGIDKSKGDYLIFVDSDDFVSQDMVSYLVSCMENNEADIAICDPV
HYYSDRQNNDLNIFSPASNVKVYETTEALCEMFYQKSFLVSAWAKIFKRELFDDIRFPVG
KLFEDSAIMYLLFEKCETIAYSDAELYAYVHRDNSITTKKFSDRDLDILEITNTIINHYG
DNLRVYTAAVSYKVSACFRILLNSPSGEKYKKVQKECLSYILQNWRNILFNNNVRLKNKL
ALISITIFNPFVKFIYSKVNRWE
>orf00797
(SEQ ID NO: 1242)
MNKYEERYQENLSKNDFYKLINKSYLSDKELQVQQVKAGIVLPPKAFETKLSNKLGLQKS
LHGKGGVVDSNGNYIELSAQKAVGMRNRVYGPYKINYDNLPIRNEKVIYLNYFIKQWGHF
LLDVVGRLWYPLLQDNDTKLVYTCYAGTETKIEGNYLEFLKLLGIDQSRLIMINCPTQFS
EVIIPESSILPGGYYTKEYKQLFSSVVENIKLDKYDVNAKMIYCSRSKLGIAKSKEFGED
GIEGIFKQNGYTSVYMETMSLEEQIKTLLSAKTIVLTSGSLAHNLLFVNKDIDVFILNKT
YRVNLHQFLINEISDATVRFVDIYRSPLPILYGYGPFLMDLTKPLANFLDDNEFVYEKGT
VLSKKDYFKYYLKWLWSYRFFLFRLNGIKEGNSEFEKSFKIIRRYYKTGR
>orf00798
(SEQ ID NO: 1243)
MSKYKELAKNTGIFALANFSSKILIFLLVPIYTRVLTTTEYGFYDLVYTTIQLFVPILTL
NISEAVMRFLMKDGVSKKSVFSIAVLDIFIGSIAFALLLLVNNLFSLSDLISQYSIYIFV
IFVFYTLNNFLIQFSKGIDKIGVTAISGVISTAVMLAMNVILLVVFDWGLLGFFIANVCG
YVIPCIYIVSRLRLWELFEIKIDKKLQWEMVYYALPLVLNILSWWVNNTSDRYIVTAIVG
IQASAIISVAYKIPQILSTISAIFIQSWQISAIKIQEDKSDTTFVSNMLLYYNALLLIIA
SGIILFVKPISNILFGISFYSAWELVPFLIISSLFNAISGCIGAIMGAKMDTHNIAKSAL
VGMIANIILNIVLTFLMGPQGITISTLIASFLIFYMRKDSVKEINSETYRAIYLSWILLV
VEACLLIYMDFIIGALIAMVINLFLLKDVIKPLYLKIFKRN
>orf00799
(SEQ ID NO: 1244)
MIVLQYFKILARFVFMFLISAVLLPFKIKPNKIVFINFNGKGYGDNPKSICEYLRTTYPD
LDLVWLARDNEGFPDGVRVVKYGTFQAFYEQASSKVWVYNVRAFARILKKRGQIYIQTWH
GASSFKLIEKQADLPINYVLEAKYDARVTDIMISDSRKQTEEFQKYFWYSGEIFEVGMPR
NDALFHYKEDYDKLNNIRKELSIHSDDYVILYAPTFRDDGDASYLDINFERLLQCVEHGI
KKKCKFLIRLHPNHSHLCNNISFNKNIINATFYSDMQELTLLADVLVTDYSSSIFDFMLL
NKPYVRYVNDLEKYAELRGVSDTYYELPDSIIKTAEELYDLLPKKIENFDYDSIKKYRNE
ILCPIFNGTASENVGGRIIQEL
>orf00800
(SEQ ID NO: 1245)
LKNNDLKIGSGAIHQISATLSQNSISGKILYCADPVVDDLYGSIVRSQIEEIGRVKEESC
NYNTIAYAMNIAERAIATDIDCIVGMGGGRVLDVCKYASFISKRPYLSIPTTAANDGIAS
PVAVLKRQDDRPKSLGAAIPSMTLIDIDVIASGPIQNIKAGIGDTISNYTALKDWELAVE
RGKDEMHGFAYLMSQNSLDALMKTKYNSITPDFIEVLVNSLVLSGIAMDFAGSSRPVSGS
EHLFSHALDYYGSTRNLHGIQVALGTVAVLKLIENSVDTVVDYLQRFEVHINPKLLGIDE
ELFIYCMQHATKMRSNRYTYLHEVDLSTDRLKQIYKELISEL
>orf00801
(SEQ ID NO: 1246)
MKALILAAGLGTRLAPITNEVPKSLVPVNGKPILMKQIENLYQNNITDITIIAGYKSSVL
TDAVTEKYPEINIIDNVDFKTTNNMYSAYLGKAAMGDSDFLMMNADVFYDASVIKSLLLH
KAPNAIVTDLGIYIEESMKVVEKNGRLVEISKQISPEETLGASIDVYKFSYEAGARFFEK
CKEFIEDKRELQMWSEVALNAILSEVEFVACPLEGRWLEIDNHEDLVAAEKLFA
>orf00802
(SEQ ID NO: 1247)
MKLTNRVDYFGADISELQNKKLFLFDMDGTIYEEDRLFEGTLELLDYIHNIGGEYIFITN
NSSKSVVDYVEKVNRLGIKAERDNFFTSAQATIVYIKENYPKSKVYCQGTKSLIKELSDA
GIDVTEQVSADIDVVLVGFDTELTSDKIRNTCEILSTKDVPFIATNPDIRCPVSFGFIPD
CGSICDMISKSVDRKPVYIGKPEPTMVDIVRKKLNYSLFETVVIGDRLYTDIMTGINAGV
TSVCVLTGEATVNDIQQDSIKPTYTFKNVKEMWKGIV
>orf00804
(SEQ ID NO: 1248)
MKGIILAGGSGTRLYPLTRAASKQLMPVYDKPMIYYPLSTLMLAGIRDILIISTPQDLPR
FKELLQDGSEFGIKLSYAEQPSPDGLAQAFIIGEEFIGDDSVALILGDNIYHGPGLSTML
QKAAKKEKGATVFGYHVKDPERFGVVEFDENMNAISIEEKPEYPRSNYAVTGLYFYDNDV
VEIAKSIKPSPRGELEITDVNKAYLDRGDLSVELMGRGFAWLDTGTHESLLEASQYIETV
QRMQNVQVANLEEIAYRMGYISREDVLALAQSLKKNEYGQYLLRLIGEA
>orf00806
(SEQ ID NO: 1249)
MTDNFFGKTLAARKVEAIPCMLEFDIPVHGDNRGWFKENFQKEKMLPLGFPESFFAEGKL
QNNVSFSRKNVLRGLHAEPWDKYISVADGGKVLGSWVDLREGETFGNTYQTVIDASKGIF
VPRGVANGFQVLSDTVSYSYLVNDYWALELKPKYAFVNYADPSLGIEWENIAEAEVSEAD
KNHPLLKDVKPLKKEDL
>orf00810
(SEQ ID NO: 1250)
MTEYKNIIVTGGAGFIGSNFVHYVYENFPDVHVTVLDKLTYAGNRANIEEILGNRVELVV
GDIADAELVDKLAAQADAIVHYAAESHNDNSLNDPSPFIHTNFIGTYTLLEAARKYDIRF
HHVSTDEVYGDLPLREDLPGHGEGPGEKFTAETKYNPSSPYSSTKAASDLIVKAWVRSFG
VKATISNCSNNYGPYQHIEKFIPRQITNILSGIKPKLYGEGKNVRDWIHTNDHSSGVWTI
LTKGQIGETYLIGADGEKNNKEVLELILKEMGQAADAYDHVTDRAGHDLRYAIDASKLRD
ELGWKPEFTNFEAGLKATIKWYTDNQEWWKAEKEAVEANYAKTQEIITV
>orf00813
(SEQ ID NO: 1251)
MILITGANGQLGTELRYLLDERNEEYVAVDVAEMDITNEEMVEKVFEEVKPTLVYHCAAY
TAVDAAEDEGKELDFAINVTGTKNVARASEKHGATLVYISTDYVFDGKKPVGQEWEVDDR
PDPQTEYGRTKRMGEELVEKHVSNFYIIRTAWVFGNYGKNFVFTMQNLAKTHKTLTVVND
QYGRPTWTRTLAEFMTYLAENRKEFGYYHLSNDATEDTTWYDFAVEILKDTDVEVKPVDS
SQFPAKAKRPLNSTMSLAKAKATGFVIPTWQDALQEFYKQEVR
>orf00814
(SEQ ID NO: 1252)
LVNCEPLEAYRQLEEAELVGCWAHVRRKFFEATPKQADKSSLGAKGLAYCNQLFSLERDW
EALPADERLQKRQEELQPLMEDFFAWCRRQSVLSGSKLGRAIEYSLKYKETFKTILKDGH
LVLSNNLAERAIKSLVMGRSKRVQWTLLA
>orf00823
(SEQ ID NO: 1253)
MNKGLFEKRCKYSIRKFSLGVASVMIGAAFFGTSPVLADSVQSGSTANLPADLATALATA
KENDGRDFEAPKVGEDQGSPEVTDGPKTEEELLALEKEKPAEEKPKEDKHAAAKPETLKT
VTPEWQTVEKKEQQGTVTIREEKGVRYNQLSSTAQNDNAGKPALFEKKGLTVDANGNATV
DLTFKEDSEKGKSRFGVFLKFKDTNNNVFVGYDKDGWFWEYKSPTTSTWYRGSRVAAPET
GSTNRLSITLKSDGQLNASNNDVNLFDTVTLPAAVNDHLKNEKKILLKAGSYDDERTVVS
VKTDNQEGVKTEDTPAEKETGPEVDDSKVTYDTIQSKVLKAVIDQAFPRVKEYSLNGHTL
PGQVQQFNQVFINNHRITPEVTYKKINETTAEYLMKLRDDAHLINAEMTVRLQVVDNQLH
FDVTKIVNHNQVTPGQKIDDERKLLSSISFLGNALVSVSSDQTGAKFDGATMSNNTHVSG
DDHIDVTNPMKDLAKGYMYGFVSTDKLAAGVWSNSQNSYGGGSNDWTRLTAYKETVGNAN
YVGIHSSEWQWEKAYKGIVFPEYTKELPSAKVVITEDANADKNVDWQDGAIAYRSIMNNP
QGWEKVKDITAYRIAMNFGSQAQNPFLMTLDGIKKINLHTDGLGQGVLLKGYGSEGHDSG
HLNYADIGKRIGGVEDFKTLIEKAKKYGAHLGIHVNASETYPESKYFNEKILRKNPDGSY
SYGWNWLDQGINIDAAYDLAHGRLARWEDLKKKLGDGLDFIYVDVWGNGQSGDNGAWATH
VLAKEINKQGWRFAIEWGHGGEYDSTFHHWAADLTYGGYTNKGINSAITRFIRNHQKDAW
VGDYRSYGGAANYPLLGGYSMKDFEGWQGRSDYNGYVTNLFAHDVMTKYFQHFTVSKWEN
GTPVTMTDNGSTYKWTPEMRVELVDADNNKVVVTRKSNDVNSPQYRERTVTLNGRVIQDG
SAYLTPWNWDANGKKLSTDKEKMYYFNTQAGATTWTLPSDWAKSKVYLYKLTDQGKTEEQ
ELTVKDGKITLDLLANQPYVLYRSKQTNPEMSWSEGMHIYDQGFNSGTLKHWTISGDASK
AEIVKSQGANDMLRIQGNKEKVSLTQKLTGLKPNTKYAVYVGVDNRSNAKASITVNTGEK
EVTTYTNKSLALNYVKAYAHNTRRNNATVDDTSYFQNMYAFFTTGSDVSNVTLTLSREAG
DEATYFDEIRTFENNSSMYGDKHDTGKGTFKQDFENVAQGIFPFVVGGVEGVEDNRTHLS
EKHDPYTQRGWNGKKVDDVIEGNWSLKTNGLVSRRNLVYQTIPQNFRFEAGKTYRVTFEY
EAGSDNTYAFVVGKGEFQSGRRGTQASNLEMHELPNTWTDSKKAKKATFLVTGAETGDTW
VGIYSTGNASNTRGDSGGNANFRGYNDFMMDNLQIEEITLTGKMLTENALKNYLPTVAMT
NYTKESMDALKEAVFNLSQADDDISVEEARAEIAKIEALKNALVQKKTALVADDFESLDA
PAQPDEGLENAFDGNVSSLWHTSWNGGDVGKPATMVLKEATEITGLRYIPRGSGSNGNLR
DVKLVVTDESGKEHTFAATDWPDNNKPKDIDFGKTIKAKKIVLTGTKTYGDGGDKYQSAA
ELIFTRPQVAETPLDLSGYEAALAKAQKLTDKDNQEEVASVQASMKYATDNHLLTERMVE
YFADYLNQLKDSATKSDAPTVEKPEFKLSSLASEQGKTPDYKQEIDRPETPEQILPATGE
SQSDTALFLAGVSLALSALFVVKTKKD
>orf00824
(SEQ ID NO: 1254)
LQIAQESSQDTDGINPPVVEEAMVFDRNDCLNQICGNIISLGIDAAFRTQVSNELIFIVV
DFTRSCCN
>orf00826
(SEQ ID NO: 1255)
MLNLMWMKIFHRNRTFLFCFLGFKVDVISIINARIVRR
>orf00827
(SEQ ID NO: 1256)
VYNSQALRQIVVVGSIDHLFKRHSSICEIFGLRKRWLSFL
>orf00830
(SEQ ID NO: 1257)
MTSIIFSAKDIFEQEFGREVRGYSKVEVDEFLDDVIKDYETYATLVKSLRQEIADLKEEL
TRKPQVSSAPSPSHPDPIDVAASSSMTNFDILKRLNRLEKEVFGKQILDNTDL
>orf00854
(SEQ ID NO: 1258)
LISIKHFFWLPLSKKMIIDIIVNKNPDRFCMIEKVKKTMAENR
>orf00858
(SEQ ID NO: 1259)
VNIDSSEFYISHITDGIFDSFLDSNRYLRNFYSVLKVEIDICCEFFVHVFKINATAE
>orf00859
(SEQ ID NO: 1260)
VNPLYLCSSDSNDFFKYTWGDNDFAKLFFNSHRMTSF
>orf00887
(SEQ ID NO: 1261)
LAQISILHFDFLSIDKHSHTVFNTLRKSLQTTLALSATSKQCFEQLAASFLVCSLIFIEY
KV
>orf00897
(SEQ ID NO: 1262)
MLYVGIDIAKNKHDVTALNVPGKTVLKPLTFSNNKAGFELLDLSLRQLNQDCLIALKLLS
DPNREQFQHDNRQVDLKILARHIHRLKKKQSDWKVQYTRCLDIIFPELDKIVGKHSEYTY
QLLTRYPNPQKRIEAGFDKLIEIKRLTASKIQDILSVAPRSIETTSPAREFEIIEIIKHY
KRLIDKAETCVNDLMAEFNSVITTVIGIGGRLGAVILAEIRNIHAFDNPAQLQAFAGLDS
SIYQSGQIDLAGRMIKRGSPHLRWALIQAAKACARFSPAFKAYLKTKLEQGKHYNVAIIH
LAKKLIRTLFYILKKSCHLTNKK
>orf00900
(SEQ ID NO: 1263)
MDTKSSCLITTGRNDSPSTCLPRVASNNDRFSSEFRIIPDFHCSKKGIHVNMDDFS
>orf00903
(SEQ ID NO: 1264)
MMSIREQDLKDIGAIIKYKNFHSPFDTFKYLKDMGFDTIDLSVLLEGFSYAYGMDWLEKF
FKENQDKLREFY
>orf00909
(SEQ ID NO: 1265)
MIPLYRTDNDITKFFTKIRNGHLAKTAGGLDDKFHEANASTSKAFDRQGVGEVNDIRDSA
GSQELRINDKRKTENILFLEIRVRIFRVPHPNDSFFSSHFLG
>orf00910
(SEQ ID NO: 1266)
VLSQGDKDITILDAGLLKNGKIGPVTKDTNDIKATDNMIENSFVLLNQQNIMLFCNQGAT
EGKTNFSPSDKDNFHNKTYFFMM
>orf00915
(SEQ ID NO: 1267)
MKIKEQTRKLAAGCSKQCFEIVDRTDEVSSKHGFEVVDETDEVSNHTYGKAKLTWFEEIF
EEYKMMGKAGQLVFFDVYRLVRQVS
>orf00942
(SEQ ID NO: 1268)
LVEIVRGGSPRPIKDYLTSEVDGINWIKIGDTEKGEKYINNVKEKIKKSGLNKTRFVKKG
TFLLTNSMSFGRPYILNVDGAIHDGWLAISNYENSLNKDYLFYILSSNVVYSQFLSLISG
AVVKNLNSDKVASILIPLPPLSEQQRIIEAIESALEKVDEYAESYNRLEQLDKEFPDKLK
KSILQYAMQGKLVEQDPNDESVEVLLEKIRAEKQKLFEEGKIKKKDLDISIVSQGDDNSY
YGNIPMNWVVIKIKDIFSMNTGLSYKKGDLSINNKGVRIIRGGNIKPLEFSLLDNDYYID
TQFISSEQVYLKHNQLITPVSTSLEHIGKFARIDKDYDGVVAGGFIFQLTPFESSEIISK
FLLFNLSSPLFYKQLKAITKLSGQALYNIPKTTLSELLIPLAPFEEQELITQKVEKLFEK
VNQLWK
>orf00963
(SEQ ID NO: 1269)
VDRTDEVSSKHCFEVVDTTDEVSSKHCFEVVDRTDEVSNHTHDKPTLTWEEEIFEEYHSP
FHN
>orf00964
(SEQ ID NO: 1270)
LDNIHIVLDSLNAVSGIQDFICDGLAIFCDQITSGCSSCK
>orf00979
(SEQ ID NO: 1271)
MKSTLGIISVGLVITYILQQVMSFSRDYLLTVLSQRLSIDVILSYIRHIFELPMSFFATR
RTGEIISRFTDANSIIDALASTILSLFLDVSILILVGGVLLAQNPNLFLLSLISIPIYMF
IIFSFMKPFEKMNHDVMQSNSMVSSAIIEDINGIETIKSLTSEENRYQNIDSEFVDYLEK
SFKLSKYSILQTSLKQGTKLVLNILILWFGAQLVMSSKISIGQLITFNTLFSYFTTPMEN
IINLQTKLQSAKVANNRLNEVYLVESEFQVQENPVHSHFLMGDIEFDDLSYKYGFGRDTL
TDINLTIKQGDKVSLVGVSGSGKTTLAKMIVNFFEPYKGHISINHQDIKNIDKKVLRRHI
NYLPQQAYIFNGSILENLTLGGNHMISQEDILRACELAEIRQDIERMPMGYQTQLSDGAG
LSGGQKQRIALARALLTKAPVLILDEATSGLDVLTEKKVIDNLMSLTDKTILFVAHRLSI
AERTNRVIVLDQGKIIEVGSHQELMQAQGFYHHLFNK
>orf00981
(SEQ ID NO: 1272)
MTSYKRTFVPQIDARDCGVAALASIAKFYGSDFSLAHLRELAKTNKEGTTALGIVKAADE
MGFETRPVQADKTLFDMSDVPYPFIVHVNKEGKLQHYYVVYQTKKDYLIIGDPDPSVKIT
KMSKERFFYEWTGVAIFLATKPSYQPHKDKKNGLLSKLPSSDFQTKISHCLHCSLKLIGH
YYQYRWFLLSPRNLG
>orf00984
(SEQ ID NO: 1273)
MDTKMMSQFSVMDTEMLACVEGGGCNWGDFAKAGVGGAAVVAALGCAAGGVKYGKILGPW
GAAIGGIGGAVVCGYLAYTATS
>orf00988
(SEQ ID NO: 1274)
MKKKILIIFVLYLIMSIFLYPLRESIWYNLFYTIAYMIAVMIYFSLIKKKEKK
>orf01008
(SEQ ID NO: 1275)
LNCKGNDHPKEFHNPNNRFDKKNSKKTKKNFILSPLA
>orf01009
(SEQ ID NO: 1276)
MKIKEQTRKLAAGCSKQCFEVVDRTDEVSSKHRFEVVDRTDEVSSKHRFEVVDRTDEVSS
KHRFEVVDRTDEVSNIYTAR
>orf01017
(SEQ ID NO: 1277)
MHSQTFQFLLMTDKTSLLHRKHRSFIRNIHSKFLILFDLLCGILSRNDSNHNPIS
>orf01021
(SEQ ID NO: 1278)
MSDVKEEVSSLSEKQLRQIDVEYAELNDSDIIERLAYLEINNNEKRIVISDIEPTKEIMS
VSDQIFEIQKNFQKIKNMFELFISDVSDFLSIKNKLESKELEIEEADVNRFMIHLLSSGK
LFVDFNENQIKQKYSKDSEEFDCIHGFASYQYDTNFAYRFCHSLRNYSQHTDLPINEVKA
VSPDDETVIIDFYIDLDYLLNSNFKWKKLKGELIKLNQETSKIDAIALVKEYFNALTELY
GNYNKLFLKLNHNTLVDIKSKLESLKLKHSRYYISKISKYDLKYNPGNYTMSPLAAFAEI
EEIYIELSKIGLVKIVNKSN
>orf01025
(SEQ ID NO: 1279)
MSKHPHYELLNLIGYGLAKFDKLFIKEFQCFSKSEFYRYVVSLGIAETTGVVKNRMDLFD
PYFDNNRKGWWQKAEVYRFRKDLIDMMFGNEDVHSYAEIVKMLLASEGKKTGITIVEKPI
VRTKFKRLQETGMEAENYFILHFDKEEKFQGGQLTDARLYGDGYDFQVDVQEYSYLAEVK
GIRKSKGRVRLTAKEFEKVKEFQSDFILSLVTNLDDIPKLVLIDNPLKHFEFKKNIIKNE
IIEYRSVEDLY
>orf01027
(SEQ ID NO: 1280)
MFIAEFTAILLNEFPVALDSLVFMGFSMKLIHDLDTHTTHSTAKMLYNMKAIKNDFSIRE
>orf01049
(SEQ ID NO: 1281)
LKHLFCHFNPLWIDEIIRLAYKDQDTKDVKSKVKIGN
>orf01077
(SEQ ID NO: 1282)
LCCNRHIANLDLEFISYHLGQVGFDTRISTGLGIFVTKIGNVLFDTDNQFASFLNVCDTC
ISLDWFGSSKAEKANQ
>orf01095
(SEQ ID NO: 1283)
MKEIAFDAFYQLYQNDQLSLVDVREVDEFAALHLECAHNLPLSQLADSYD
>orf01098
(SEQ ID NO: 1284)
MCLICQRIELIKAGQNPYFVKELETGYVVIGDYQYFKGYTLFLAKDHVTELHHMETSVKL
RFLEEMSLVQEAVAKAFEAEKMNIELLGNGDAHAHWHLFPRRAGDMKSHGLNGRGPVWWV
PWEEMAAEDCQVQSPELEEMIKILSHELEKYLA
>orf01099
(SEQ ID NO: 1285)
MKKRYVILSGLLALTLAACSQEKTKVEENTQKTEQSSQPEGTVGSKSQASSQKKAEVSNK
GSYYSIQGKYDEIILANKRYPLSKDYNPGENPTAKAELLKLIAAMQAEGYPISDQYSGFR
SYETQAKLYQDYVNQDGKEAADRYSARPGYSEHQTGLAFDLIGTDGDLVTEEKAAQWLLD
HAADYGFVVRYLKGKEKETGYMAEEWHLRYVGKEAKEIAASGLSLEEYYGFEGGDYVD
>orf01104
(SEQ ID NO: 1286)
MKTKEQTRKLAAGCSKHCFEVVDRTDEVSNHTHGKATLTWFEEIFKEY
>orf01105
(SEQ ID NO: 1287)
MDFFFMNEVKEQVLFRDNHSEHIFWIEGVSDFMIKVNTALW
>orf01109
(SEQ ID NO: 1288)
VCFLGFQTILANPSKPQRQLPFLIFILDFFNYKHHKFLS
>orf01124
(SEQ ID NO: 1289)
MEELVTLDCLFIDGTKIEANANKYSFVWKKTTEKFSAKLQEQIQVYFQEEITPLLIKYAM
FDKEQKRGYKESAKNLANWHYNDKEDSYTHPDGWYYRFHHTKHQKTQTDFQQEIKVYYAD
EPESAPQEGLYMNERYQNLKAKECQALLSPQGRQIFAQRKIDVEPVFGQIKASLGYKRCN
LRGK
>orf01126
(SEQ ID NO: 1290)
MHIHYNTNQTTLPLEISSFLPQDHLVFTIEKVVNTLEDCHFHAFYHAFDRLSYHLKMLVS
TLLFAYSQGIFSGRKIEKWKS
>orf01129
(SEQ ID NO: 1291)
LRLWVIFVIMKVIKSYDTLNDYYRKLFGEKTFKVPIDAGFDCPNRDGTVAHGGCTFCTVS
GSGDAIVAPDPPIREQFYKEIDFIHRKWPDVQKYLVYFQNFTNTHEKVEVIRERYEQAIN
EPGVVGINIGTRPDCLPDETIEYLAELSECMHVTVELGLQTTYEATSDLINRAHSYEL
>orf01131
(SEQ ID NO: 1292)
VETVKRLRKYPKIEIVSHLINGLPGETHEMMVENVRRCVTDNDIQGIKLHLLHLMTNTRM
QRDYHEGRLQLMSQDEYVRVICDQLEIIPKHIVIHRITGDAPRDMLIGPMWSLNKWEVLN
SIEMEMRRRGSVQGCKAVKQEFENEKTT
>orf01143
(SEQ ID NO: 1293)
VQVCVFTNFCFFHCFSSLANCRLFNLRGICLPCISYQ
>orf01152
(SEQ ID NO: 1294)
VFKKDRFSIRKIKGVVGSVFLGSLLMAPSVVDAATYHYVNKEIISQEAKDLIQTGKPDRN
EVVYGLVYQKDQLPQTGTEASVLTAFGYLSGDILKTLGLDTVLEETSAKPGEVTVVEVET
PQSTTNQEQARTENQVVETEEAPKEEAPKTEESPKEEPKSEIKPTDDTLPKVEEGKEDSA
EPAPVEEVGGEVESKPEEKVAVKPESQPSDKPTEEPKVEQVGEPVEPSEDEQAPTAPVEP
EKQPEAPEEEKAVEETPKPEDKIKGIGTKEPVDKSELNNQIDKASSVSPTDYSTASYNDL
GPVLETAKGVYASEPVKQPEVNSETNKLKTAIDALNVDKTELNNTIADAKTKVKEHYSDR
SWQNLQTGVTEAEKVAANTDAKQSEVNSETASLKTAISRLNTDKVELENQLKIAQGKTET
DFSMESWTVLSTAKNKAQEVKDNGTATQEQINEAEKSLKTALADLSVDKTALGSAIDTAT
KKNKENYTNQTWAELETALTAAKSVNTNESKQSDVNEAAEKLTATMEKLVELSEKPRLTL
SIEKRDIDRKATVTYTLENPANTQIKSITATLKKGEEVVKDFVLTEENLKTNHLTALFEK
LDYYKEYTLSTDMVYNRGNDDETESISEELIQLNLKKLELKDIQTVSLMKFENGQESQVT
HLSDKPTDLSKLYLKVTSSTSKDAVLAVSSIEEEIIENKKIFKIHADTPELVVRKKDGSL
SKGFDYYMERVIPHDGDIYYDFKDLISAMTSNPTGTFILGRDISSRNVKPDGNGKSYIKG
EFKGKLLGTNDNVRHSIFDLEYPLFDTIKSGVVKDIDFKHVNMVFPDSNQGDNVATIARV
IKDKTKIENVNVEGYLEGRDHVAGLVNNLEGNSEIENVSFTGKIKSKGGNSITAGIAGRN
ILSRVKRAYVNADIEVHRSSNSSMLVAVNGINADASGGWGTWGRLTESVAKGTLETKQGG
QAGGASSTVWPYGAIDNVVSYAKVTKGKELFGSDGDLNYDWFMKKISNIFGVQGISSGDS
GSDSKFTRISEEEANQKVASYNITAPNLMSDSSLLVDRLNESWKNTDQFESIQDYQAQNQ
LIYQNLTKFTPYYNKEFIVHEGNALTPEQEILKTKKIKSIVGLKGTEFVVDGSDIDTIML
HFEDGSQKRYKVTSTGKFSITNLPEYQVEDLNVVYTSEHIVHPLDSSLINNLVEELKKVE
LYTESTYQVLGIDKDNANKLNRTKRLFLDESLDAVKTQLPTFVKTMFENEWLHINGESSG
AVAALRQKIMDNKTAILLALTYINRYYDVKFSDYNIKKLMLFKPTFHGEKIDLLDRLIRL
GSSGENRLKGSENAETFKQLFASETKQKDLVTYLDYNRSLLTNYQTTGEWFKETTKDYIQ
FEERPSLVEEIKDAKYRVYDNLTAPYYQGYILPLLTLKNTHLAILSNYSTMTFVSREKRP
NWKNEDFDKWVKYVATAHRNHVDTWYKILPDNIKGKMVKENVTAVWEGLSIPGSEWVDQN
AVDRKGRDYAPAREFFNLVGGPMGGWYAYHGYGAHAGGRNRVNYEVFDVLSEYGISVFTH
ELTHVNDTWIYLGGYGRRENMGPEAYAQGLFQSPVPGQPGWGALGLNMAFERKNDGDLIY
NASPTQFENRKELDSYMKNYNDTLMMVDYLEGDAVISKGKEAITKWFKKVEPKVVSQTAQ
YDTVRQLTAEEKEKLSVSSVDDLVDQGLMSDRAVGNNTYNPADFETSYIAIDYMTGIYGG
GKNSVGSPGALMFKHNTFRMWGYYGFEEGVLGYASNKFKQASRDEGHAGLSDNFIISKIS
KGEFLTMEAFKKGYFKKVVEELKTKGIRPVTINQKTYSTFEELQEGFKQAVERDLKKNQL
DERETRNFKFQVFRQLLQQTDSFKTSIFR
>orf01156
(SEQ ID NO: 1295)
MKSKIVLGASLAIATLSLVSLVEIEGLSPFLIENVSANTHSANKVINHKVSIYLENADEG
KGLTVNFSTDSVSPNLFDEFEKKSGITITTMLVNAKTGEVVEKRLTPSVFLRSNDLTSGT
ISSFIFSEYPDGEYKYVVSKGDFIDPKTQFKHQYRGESPVFRIRNRKYVELGTTDKKLDE
RRDNSVYKDGVVEHKVNLSLTSYQGGNGVTAIFSTDSVNSNLLNSFGEKAKKVLIRSKLI
NVKTGEVIDETFSPKVSLTSKILKSGSTAVFYFIDLTDGEYKYVAYESQQYTDPQTTLTH
QYRGESPIFSIKDGKFSGLVSASKPDENPKPTPKPDEKPKPSAPQQEKTKPTVQSGWVGS
SYYQNGKKVTSKWIFDKKYNSYFYLDASGNYVQNAWVGNYYLKSGGYMAKSEWIYDKNYG
SYYYLTSEGSYARNTWSGNYYLKSNGKMAKSEWVYDSNYKSYYYLTSEGSYARNTWVGNY
YLKSNGKMAVNERTPDGYRVDGSGKWVK
>orf01157
(SEQ ID NO: 1296)
MSACTVCAEKGRTPDLSIVDNVPIVENAKAHENNFFYSSDITYYPIF
>orf01158
(SEQ ID NO: 1297)
MAKYYIILPKDAEIYKTWRGTVNIPIIDATKTTPELSYFKEDHRNYIANENKSGANYIEW
KGTVEEFKEAIKKLTDKKSTTATPKKDEKPTPKPDEKPKPTPTVQSGWVGSSYYQDGKKV
ISKWIFDKKYNSYFYLDASGNYVQNAWVGNYYLKSGGYMAKGEWVYDATYQAWYYLTSDG
SYAYSTWQGNYYLKSDGKMAVNEWVDGGRYYVGADGVWKEGQASTASSSNDSNSEYSAAL
GKAKSYNSLFHMSKKNVCIDN
>orf01179
(SEQ ID NO: 1298)
VSRWDGHSDKGEAPAGKTSYAWIWTKWGEQVAFYCDYD
>orf01193
(SEQ ID NO: 1299)
MKSKKGGELRIAVFGDKKPFGYVDNDGSYQGYDIELGNQLAKDLGVKVKYISVDAANRAE
YLISNKVDITLANFTVTDERKKQVDFALPYMKVSLGVVSPKTGLITDVKQLEGKTLIVTK
GTTAETYFEKNHPEIKLQKYDQYSDSYQALLDGRGDAFSTDNTEVLAWALENKGFEVGIT
SLGDPDTIAAAVQKGNQELLDFINKDIEKLGKENFFHKAYEKTLHPTYGDAAKADDLVVE
GGH
>orf01194
(SEQ ID NO: 1300)
MKLFKPLLTVLALAFALIFITACSSGGNAGSSSGKTTAKARTIDEIKKRR
>orf01231
(SEQ ID NO: 1301)
MYQDEAGFGRISKLGSCWAPIGVGPHVHSHYIREFHYCYGAVDAHTGESFFLIAGGCNTE
WMNAFLEELSQAYPDDYLLLVMDNAIWHKSSTLKIPTNIGFTFIPPYTPEMNPLNKCGKR
FVNVDLRIRPFELWKMS
>orf01233
(SEQ ID NO: 1302)
MVTATTCFLKERVEFELLIFFYISPNRCLITVYSVLNL
>orf01234
(SEQ ID NO: 1303)
MDTPDENGYVADDYRITYLEAHIKAMRDAIYKDGVDLLGYTTWGCIDSVSAGTGEMNKRY
GFIYVDRDNVGNGTLKCSKKKSFYWYMSFIAMV
>orf01255
(SEQ ID NO: 1304)
MFLGMIGNISIILQFFGITIIVKIDNQARAIDFFKHDKSSF
>orf01257
(SEQ ID NO: 1305)
MFSLNFFDDSVFLSIKIAHKGCFQLLDMTNPNFFNKFFLAQASDQLLHFLSWNIEL
>orf01266
(SEQ ID NO: 1306)
MTEPDFWNDNIAAQKTSQELNELKNTYNTFHKMEELQDEVEILLDFLAEDESVHDELVAQ
LAELDKIMTSYEMTLLLSEPYDHNNAILEIHPGSGGTEAQDWGDMLLRMYTRYGNAKGFK
VEVLDYQAGDEAGIKSVTLSFEGPNAYGLLKSEMGVHRLVRISPFDSAKRRHTSFTSVEV
MPELDDTIEVEIREDDIKMDTFRSGGAGGQNVNKVSTGVRLTHIPTGIVVQSTVDRTQYG
NRDRAMKMLQAKLYQMEQDKKAAEVDSLKGEKKEITWGSQIRSYVFTPYTMVKDHRTSFE
VAQVDKVMDGDLDGFIDAYLKWRIS
>orf01267
(SEQ ID NO: 1307)
MLQSNQVQNFHHSSFDITAIFPDYFHSVSNIFIDSFLW
>orf01299
(SEQ ID NO: 1308)
MQVIKRNGEIAEFNPDKIYQAILKAAQTVYVLTDDLRQNLAQVTKKVVLDLQEAKVERAT
ISMIQSMVEHRLLGAGYITIAEHYISYRLQRDLERSGYGDHIAVHLHFEQIR
>orf01305
(SEQ ID NO: 1309)
MKLKLCIIGFFFCLIATIGLVTISDTEIPIPLPIDGAFSIQGKSNLSNNEIYEMVRDLSK
TEKVTIYKPIVQSSGQLKYVNFDDVNNEQLKSAPIVGMYYTLGKMDVDSLKPLTMTGLQT
VYMAYPWYIGGILQFTGTLRILLMGSIYLTLLVVLFVVRTRQIKEGVIRRSLGLPIYDLR
REYGISLIFELIMMALLMISYSSFLGNGFFTYSSKLFFSLLLTNFILFQIIDLITFVLFW
LTIQIEKPIEIIKNKAKNKLIFVVWLAIISIIILVSGVFLQETKSSQSSINIQIQNLVPW
DTVKDWRRIEFLGIESNSTKNREVNDSDGQYLQIVAALKNLDFLYIERSSAYVPDFMKTS
HVIENFSKQLENDGITNPEINKELIYINQTGANLQNKVNGTNYHLLDNKIATIYIPEKWK
ENQKSIENTVVAEQFIGTNYTREQLAVQIIPDGEKIFYFNEDADNNLKMKDILPLANVAD
SKDNIVVVLDTDKMMENNKFSLASNILYKSLFSPEAVKKINEMTVLLNFSMNPVDVYQIV
KLKIQSLEHQILLSQILQKIIYSIVFILIYQYVQLFITLKQNEYVKKIILGLSKTYIAIS
SLKYFMMTITMVILFTFLMTGQIELLYIGAASLLVLMLSIIMSFRKLSESYTKILKGDES
>orf01306
(SEQ ID NO: 1310)
MTIDLLNVSKSFGSKKIFTDLNLIFESGKSYALIGGSGSGKSTLLNIIGRLEKIDSGNVL
VDKQDIWKIKERTFFKNTVGYVFQNYSLIDNKTVYDNLSLITKDKKTITDVLEKVGLSSD
YLHQKIYELSGGQAQRVAIARMLMKPRKIILADEPTGALDGEIGKEIIRLLLNETAEDKY
VIIATHDPAVYNEVDVIIDMKDIGYKV
>orf01307
(SEQ ID NO: 1311)
MKKKIYIALIFVTGVLAIFFFGKQMITKENINKPTVELTIYTLSSSDTEKWNKVRQVETE
EAIYFITVKEVSSSEEVFSNIIANGAATGFGVREEEVKKFNNGLGDTIEDSKHNKLIEIE
FFTFSDDGAGFVVANFDYGKEELNSQKKDIKELYKKIYESFKEKNK
>orf01317
(SEQ ID NO: 1312)
MKIKEQTRKLAAGCSKHSFEVVDETDEVSSKHSFEVVDETDEVSNHT
>orf01324
(SEQ ID NO: 1313)
MELFKTWKKNMVLYGLKSQIGTVYRNNDRTTSFYDVGNFLYLAGELDSRFWEDFVRKYGL
DYKIIISENTNWQDFLHRKVGLNSFTRYSFKDKANFQVEFLNNLVTHLEEGYNIVPIDNH
IYNCFSTEEWSQDLQGDFESYQDFVLKGGFGFVILKNNELIAGISSGLVYRKAVEVEVAT
RPNEQGNGFAKKLGAAMILESLNRDMFPLWDAHNEASKKVAEFLGYELSEPYEAFELEEI
LI
>orf01369
(SEQ ID NO: 1314)
MKVIDQALLEKVIIERSRTSHKGDYGRLLFLGGTYPYGGAIIMAALAAVKSGAGLVTVGT
DRENIPALHSHLPEAMAFSLQDQQLLKEQLEKAEVVLLGPGLRDDAFGEDLVKQVFAGLR
QNQILIVDGGALTILARTSLSFSSSQLILTPHQKEWEKLSGITIEKQKEDATASALTSFP
QGTILVEKGSATRIWQAGQSDYYQLQVGGPYQATGGMGDTLAGMIAGFAGQFKQASLYER
VAVATHLHSAIAQELAQEQYVVLPTEISNCLPKVMKRYV
>orf01376
(SEQ ID NO: 1315)
VLDSKEELKESENDAPKLETPLREEPRLAPQTLPEASEVLENKREESKVEITEPAQADDI
RKVVGELAKDISITKLYMTGHSLGGYLAQIAAVEDYQKYPDFYNHVLRKVTTFSAPKVIT
SRTVWDAKNGF
>orf01404
(SEQ ID NO: 1316)
MGRKPKNRPEERTELEHLQAENEYLRAENAILKKLRELRLKEEKEKEERQKLFKN
>orf01417
(SEQ ID NO: 1317)
VVLSTSAILVACGKTDKEADAPTTFSYVYAVDPASLGYSIATRTSRTDVIGNVIDGLMEN
DKYGNVAPSQKDYDLNSTGWAPSYQDPASYLNIMDPKSGSAMKHLGITKGKDKDVVAKPG
LDKYKKLLEDAVSETTDLEKRYEKYAKAQAWSTDSSLLMPTASSGGSPVVSNVVPFSKPY
SQVGIKGEPYIFKGMKLQKDIVTTKEYNEVFKKWQKEKLESNSKYQKELEKYIK
>orf01421
(SEQ ID NO: 1318)
LNFDFFIFLAHFIPLFTFSILQENPKTSKKKLYIRLL
>orf01428
(SEQ ID NO: 1319)
MRLSMKLIHDLDMHTTHSTAKMLYNMKAIKNDFSIRE
>orf01442
(SEQ ID NO: 1320)
LIRIIRNIYRSGEGNTSVFQSFIDQINSNQFCYGSNFDRLRCILLIENFTSICLNSNRMF
SGNGKILSNSSRSTP
>orf01453
(SEQ ID NO: 1321)
MSNYRRTSKPKTEHIKKGFTVFQKTITTIGSILGLITAGITIMNALDNNNKNTKKEPTTS
QTTTFVKEIQKESPQENTTPNKENNTSQEKTQQEETPKSSVKEEKKEDQKTATQDSTTPA
TSKPATENEKQPNTPTSENNTQ
>orf01457
(SEQ ID NO: 1322)
MNQSYFYLKMKEHKLKVPYTGKERRVRILLPKDYEKDTDRSYPVVYFHDGQNVFNSKESF
IGHSWKIIPAIKRNPDISRMIVVAIDNDGMGRMNEYAAWKFQESPIPEQQFGGKGVEYAE
FVMEVVKPFIKHKTGWFDGMMTTGCSMGAYHALNFFLQHPDVFTKVIALSGVYDARFFVG
DYYNDDAIYQNSPVDYIWNQNDGWFIDRYRQAEIVLCTGLGAWEQDGLPSFYKLKEAFDQ
KQIPAWFAEWGHDVAHDWEWWRKQMPYFLGNLYL
>orf01466
(SEQ ID NO: 1323)
MSSIHTKNSSLKSKSRFNEMFGDPLNNNKKFAVKTGQQCFKFSSGKFLDKHDRVFEGYPA
YGGNGIAWKSRKYLIDNPTIIIGRVGAYCGNVRTTHGKVWISDNAIYIKEFKNSDFNLVF
LLELMKVIDFSKFADFSGQPKITQKPLENQKYILPPLALQNEFADFVALVDKSQFACEIA
IKVWRNSLKFSII
>orf01476
(SEQ ID NO: 1324)
LVYAPFSFNILLDYITFDFKILLFSVFLAINRFHNDFIQFLL
>orf01479
(SEQ ID NO: 1325)
MSEYSGLSFFEVALAEFLDIVSAVYLEDADGIIVNLWGILDK
>orf01490
(SEQ ID NO: 1326)
MEGVAKGRIGRKKNNGIDNRCCHKKRNGRVTWNLFFQKTIDDGDDSTFTRREKYTDKGPK
KDSPPTISREKMINLVRCDINFNQP
>orf01493
(SEQ ID NO: 1327)
VDKTDEVSSKHCFEVVDRTDEVSSKHCFEVVDRTDEVSSKHCFEVVDRTDEVSNRTTVRR
S
>orf01495
(SEQ ID NO: 1328)
MTEFMSDNFPKNLHTQFLINLGIKIQMPIFGEKSPTCRT
>orf01503
(SEQ ID NO: 1329)
VVIGVASATTNIWIIFLSGFTAILAGAFSMAGGEYVSVSTPKDTEEAAVSREKLLLDQDR
ELAKKSLYAAYIQNGEFKTSAQLLTNKIFLKNPLKALVEEKYGIEYEEFTNPWHAAISSF
VAFFLRSLPPMLSVTIFPSDYRIPATVLIVGVALLLTGYTSARLGKAPTKTAMIRNLAIG
LLTMGVTFLLEQLFSI
>orf01535
(SEQ ID NO: 1330)
MSFKNNWIDKEGRVFIYFTVEEIMKRRNISKPTAIKTLDELDIKKGIGLIERVRLGLGKP
NIIYVKDFMSIFQVKENDLQKSKNLTSEVKDFNLRSKENELQEVKNLDSNYIENNKSKYS
KREYSFGENGLGTFQNVFLAAEDI
>orf01543
(SEQ ID NO: 1331)
LLHIRVCKTFDRIPYCMLALFLSKSIGLTILLHKVKTVIFIDDQSNDKTCKICIHISFFR
IKLSQQCQLSFSVYF
>orf01547
(SEQ ID NO: 1332)
MTQEDALIVISHIKVLSIVPNRCLKPLDKTFSLYNWIFLSQKYILLQANFLKISRVQLQ
>orf01552
(SEQ ID NO: 1333)
VTTHDEPVYEKHGVLHYAVANIPGAVARTSTIALTNVTLPYIEALAGKGFAQAISEDEGL
RQGVTTYQGYLTSLPVAQGLNRDYTDINDLV
>orf01553
(SEQ ID NO: 1334)
VFFIDGFIVRCHTVSCFDNATLVNSNVNDTEPGRICLTISSVTNSGAFAPGMRMAPITTS
ASLTLASMLNELDIRV
>orf01555
(SEQ ID NO: 1335)
LSTKTKGDAGSMCTDDTITDDSYFSFGTPGTPPGRTPEPPACLVRK
>orf01556
(SEQ ID NO: 1336)
MLIGIPKEIKNNENRVALTPAGVHSLVSRGHRVLIETNAGLGSGFTDADYQKQGAEIVAT
AGEAWAAELVVKVKEPLNSEYGYLRDDLLLFTYLHMAAAPELADAMLAAKTTGIAYETVR
DNQGQLPLLVPMSEVAGRMAV
>orf01576
(SEQ ID NO: 1337)
MASRNVLSMEPKFLLAGHFKGQFLILKIVSSDIDDGFAIAC
>orf01577
(SEQ ID NO: 1338)
LSSADKTCLNQFFTDFSDFFQSSLVEDGFYTFQIENSGFGFFNQISQVFDSFFEFLIPFK
IALGILVGSQSLIKRNHDRLVGIVVV
>orf01578
(SEQ ID NO: 1339)
VSVFLKFIFNTTNQFTGLLFDAVALSLILIVGVQQIRKICKRLSHLICKRNWTEGSLSQA
WLGFLIEKISESGKFFTNQYPFQFICSIASQTLKEALKIFCC
>orf01579
(SEQ ID NO: 1340)
MVSNLVFIGNCNFHNTVIFHLLNRLNQGPLQILSQNHDKGRRLSWIFKSRLGQLNASKNW
MGRKEQAMALAIAADLQDQLLFKRLIDFLDATIH
>orf01599
(SEQ ID NO: 1341)
LLYNPVEKTRVHIKKGIGKLQYLFTRLFYLIFVSTDYISYGSSSEG
>orf01630
(SEQ ID NO: 1342)
MRSYITLICNLNNNLFCLNSFFLTNLVWSQIFSLLSVFITVYI
>orf01631
(SEQ ID NO: 1343)
LLEITLKSPYQFAHILFQSTIVPHGGHYHFIPESDLSAGELAATYVFNPNDIVRDTGDAY
IVRHGDHYHYIPKSSLNNPPSHSNTEEVGSSSSSVLSNPSLHVHHEEEDGHGFDANRIIS
EDSEGFVIPHGDHNHYIKVQTKGYEAALKNKIPSLQSNYQPGTFDEKAVLAKVDQLLADS
RSIYKDRLS
>orf01664
(SEQ ID NO: 1344)
MARLEPAKIAKIVLGILLYIIDLIKSSFVLPIPKAAKKSLILISFVPSFNDKNIVIRRPR
QITKIMPRFICFLFRIFACIS
>orf01680
(SEQ ID NO: 1345)
MELSAIYHGPESEYAYLYKDKKLHIRIRTKKGDIESINLHYGDPFIFMEEFYQDTKEMVK
ITSGTLFDHWQVEVSVDFARIQYLFELRDTEGQNILYGDKGCVENSLENLHAIGNGFKLP
YLHEIDACKVPDWVSNTVWYQIFPERFANGNALLNPEGTLDWDSSVTPKSDDFFGGDLQG
IIDHMDYLQDLGITGLYLCPIFESTSNHKYNTTDYFEIDRHFGDKETFRELVDQAHHRGM
KVMLDAVFNHIASQSLQWKNVVKNGEQSAYKDWFHIQQFPVTTEKLVNKRDLPYHVFGFE
DYMPKLNTANPEVKNYLLKVATYWIEEFNIDAWRLDVANEIDHQFWKDFRKAVLAKNPDL
YILGEVWHTSQPWLNGDEFHAVMNYPLSDSIKDYFLRGIKKTDQFIDEINGESMYYKQQI
SEVMFNLLDSHDTERILWTANEDVQLVKSALAFLFLQKGTPCIYYGTELALTGGPDPDCR
RCMPWERVSSDNDMLNFMKRLIKIRKYASVIISHGKYSLQEINSDLVALEWKYEGRILKA
IFNQSTEDYLLEKEAVALASNCQELDNQLVISPDGFMIF
>orf01688
(SEQ ID NO: 1346)
MGQEIKLIRKQFRITRQEEKQIKEMMREQKVDSFSEFLRQNLLKKNYQDRIFESWFSLWQ
SQKFEQISRDVYEVLVVARENHQVTQEHVSILLTCVQELIAEVNQVQPLSREFREKYMG
>orf01689
(SEQ ID NO: 1347)
MVYRYRTNLKKVFLTDPELHQLNERIAKSNCQNFSVYARKVLLNPNMSFVTINTDTYDQL
VFELRRIGNNINQIARAINQSHLISQDQLQELSKGVGELIKEVDKEFQV
>orf01690
(SEQ ID NO: 1348)
MVVTKHFATHGKKYRRRLIKYILNPDKIDNLKLVSDFGMSNYLDFPSHTEMVEMYNVNFT
NNDKLYESRNDRQEKHQQTIHAHHLIQSFSPEDNLTPEEINRIGYETMMELTGGRFKFIV
ATHTDKDHVHNHILINAIDRNSDKKLIWNYALERNLRMISDRISKMAGAKIIEKRYSYRG
YKKYRESSHKFELKQRLYFLMQQSK
>orf01691
(SEQ ID NO: 1359)
MMTDRAMTKPIRGRQLSKRDLYDEEFFRTHFAKQEIESRLEFLLNRVNSLEELITKAKEL
NLTIDLKQKNVTFILKENNQKISLGHQKISDKKLYDVKFFQDYFKNNEVIASEGLENLQE
QYHAFQEERDKDKVSTEEIEEAFKTFKKPLRHLRKNEIPFVNLKWNLQRTK
>orf01692
(SEQ ID NO: 1350)
MEEENHNKYKVYIRETSSYFVYNKENMDNNCFIKGRTLIRQLSNDSQKLPYRRPTLKSLQ
EKISEINLMIELSNTNKQYQEIKDELVLEIAEIDMKLEETQEKIATLNKMAEVFINLKSE
DEIGRKLAKYDFDQMNMTESIMLDRLNTDILKLQQELGNEINKYEEIARRLDLFVKIINT
NKFTVLKFHENALLE
>orf01693
(SEQ ID NO: 1351)
MNSKLGITLRKVRKGKQISLCSVADEHLSKSQISRFERGESEISCIRLINILDKLHITLD
EFLVLHNDDYTSSESFANLVQYIRKQYSSQSINNIACLLSDTSDYTLNSFEKTMVKSILH
TMDSNIIPSDEELLHLTDYLFKIEKWGYYEIILLGNCVRTINYNSYFLLTKEMLNNYIYS
SLNKTNKQIVSQLAINCFILSIDKEEFSNCSYLISKIKTLLDNELNFYEQTVFLYATGYY
EFKRQLSSGIETMKQAIQVLDILGEDKLKLHYTSHFDKLVNNK
>orf01694
(SEQ ID NO: 1352)
MSLSYYYEINPSTDILKCIEELLYKEDKCFNNILKNWKDIRRNHNDSFPNFWCYGAPGIL
LARKEIFDKTNIGNNDLSIIKNVLTNVEKIRELNLCHGSVGTISCLDAILKDEENLLIKE
SIDFYFDNVVSQVIKPELSTDLNTMNTFSFMLGVSGVVYEISRKQDDRLLNVLLLELRGH
DD
>orf01695
(SEQ ID NO: 1353)
MMTRKVPNIEQMSQIECGLCCCLSILHFYKSKETLLDLRRDIEKGRDGYSIGDLKQLLNK
RNFDTGSYQVKDVNKISELPLPLIAFWDNQHYVVIYKVKKNKVYIMDPSKGYINYEFKEF
SKHFSNIVLLSFPNENYQSLKSQFPSPWIRVFSSFSKVKGRLILTLLFSIISYLIILSVP
VMTSKFINSALGNTFSFQTSFLILFSLLCLYLISILARSMGILFSNIFFSRDIESFTFKH
LLKLPYSFFELRAKGDILYRISSLSGFRELFTNQVVGGVVDIGTILSVVIYMFLSSKTLS
IIALILSLINFLFLFSTRKIMYDTVNRELQEQSLIYSVETEALNTISSIKISGLEDEIYE
NWSKYLKNVLTKYKKRSIVHILYNSATNVFQLFAPIIILIFGLDNVLNGKILLGEVVAFQ
TMASILFSSEISIFNAYTQYILAAGYLNRVNDIWLENEENVENGLKKCSLEGRIDIKDLS
FSYSKDSAPVIENLNLTIEPGQRIALVGQSGSGKSTLSKILSGLYKIDTGKILFDGVNIN
QIDKKILSQNLGVVPQDSFLLNRSILDNITLKNEVTSQKIEEVCKAVQIYDEIMAMPMKF
NTIISEMGSNISGGQRQRIALARALINNPSIVILDEATSALDTINEERITKYIQSQGCTQ
IIIAHRLSTIKDADIIFVMKGGKIVESGNHKYLMDLGGEYYSLYTKRK
>orf01696
(SEQ ID NO: 1354)
MAIVEIINLTKSFKDIEVIHNTSFYLNKGKVYGFVGPNGAGKTTIIKMILGILKPDSGKI
TIFNQTVEQNSENILSRIGLVLGPSFYGHLDAYKNLKLIANMKGLSLDTERLNEYLSMVG
LKDVKKKKVKNFSMGMKQRLSIAASLLGSPEILIWDEPINGLDPQGVIEIRSLIRFLQEK
KGITFLISSHILSELDKVISDIIIINYGKVEFFGSCHYLLQKYNCRNLEEAYLACLAGGE
YD
>orf01697
(SEQ ID NO: 1355)
MIKLEFLKQKKSILWFVLIFPIILNVLLYIDLTFRYRGYLLVHQNELALSNWQLIFKEQT
IFYFSELFYLVLSLIIYEVFAVEFKNDAWLTVISLPFRNKYTINSKLLITVVYTFTFWLS
DYISLYVIGKAIDNSLEIGLIFFLKTFTIQLISSLMIMLLYFLTLVLIRKISGIIPIGII
MMILTISIYYNDYNFKIYLPFTYLSHAFRVTESQFYMILLSNIIIIVLFYILIRKLNERS
FEMKL
>orf01698
(SEQ ID NO: 1356)
MKLLKNELINSKIFLFIIVDICIQILVILAIKTYILDISALYSELDYNKYWYILHTLIYM
LMIFPIQILYQNLREALIEDNNHGWNIMVINTNNLVKIIYIKVTINIVRCFICYFVYTIF
SLIQLGGMGTDMLLTNIIFPNIMSFLLFLPIAIFMQICCIRFDSILAKALPNILLILIVL
ITFQSDWNIFIPATYYYTEIQSTTNLGIKLLVCIWIMGFEFFLLPKLIKLKEQNLV
>orf01725
(SEQ ID NO: 1357)
MRFSAFKIFSNSVCKRIITKGLGFRALLLYTISKVKLREDILVSQSIVPVEIPQYCRFDS
KKRNGILFNVRIANLKFTFFRLDFLRNKIWYSSSMNDEASKQLTDARFKRLVGVQRTTFE
EMLAVLKTAYQLKHAKGGRKPKLSLEDLLMATLQYVREYRTYEEIAAVFGIHESNLIRRS
>orf01753
(SEQ ID NO: 1358)
MHTKSRTIKSLITQFTAILLYELPLALDSLVFMGFSMKLIHDLNTHTTHSTAKMLHNVKA
IKNDFSIRE
>orf01776
(SEQ ID NO: 1359)
MIKIYFTKFSENHNPFCKIFEIIFTNLIFQSILNKNKKNPLRQGEANVV
>orf01783
(SEQ ID NO: 1360)
MSQVKGLCVLDVDGTLILEEVIDLLGREAGHEAEISQITSRAMRGELVFESSLRKRVSLL
EGLPILVFDNVFNSIHLSLNVPEFISILQKNGILVGLVSGGFTPIVGEISKIPWYCLFHC
QPA
>orf01784
(SEQ ID NO: 1361)
MLKSAELGIAFCSKEMLKKEIPHHVDKRDFLEVLPLIDCLE
>orf01789
(SEQ ID NO: 1362)
MFGNWFFKAFVCSLERLAQDRTMNWFSCIGNKNTVAFVPILIGCFA
>orf01804
(SEQ ID NO: 1362)
MEKYFGEKQERFSFRKLSVGLVSATISSLFFMSVLASSSVDAQETAGVHYKYVADSELSS
EEKKQLVYDIPTYVENDDETYYLVYKLNSQNQLAELPNTGSKNERQALVAGASLAALGIL
IFAVSKKKVKNKTVLHLVLVAGMGNGVLVSVHALENHLLLNYNTDYELTSGEKLPIPKEI
SGYTYIGYIKEGKTTSDFEVSNQEKSAATPTKQQKVDYNVTPNFVDHPSTVQAIQEQTPV
SSTKPTEVQVVEKPFSTELINPRKEEKQSSDSQEQLAEHKNLETKKEEKISPKEKTGVNT
LNPQDEVLSGQLNKPELLYRDETIETKIDFQEEIQENPDLAEGTVRVKQEGALGKKVEIV
RIFSVNNEEVSREIISTSTTAPVSRIVEKGTKKAQVIKEQAETGAEHKEVQSGAIVEPAI
QPELPAAVLTDKGESAVQPELPEAVVSDKGVPEVQPALPEAVMTDKGDPEQVEPLPEYTG
VQAGAIVEPEKVEPEYAGVQAGAIVEPEQVAPLPETTGVQAGAIVEPEKVEAPKEYTGVQ
AGAIVEPEKVEAPKEYTGVQAGAIVEPEKVEPSKEYTGVQAGAIVEPEQIAPLPEYTGVQ
AGAIVEPEKVEALKEYTGKIEQPSAEDTKPNNENTNTPEEMSIQKKSSALINMNFITDSS
KVTGVGSATFIAPNVLLTVAHNFINNSTDNTTGEFRGDKSKNVYEWVTPDGQKGTFTANN
IHFYNKKDYPKGFIYDLAVIKLPETTGREHVELVKNYSKVNLNDKLNVHGYPAGKYTHLK
DATVEMEQEYANNTYGVQYQGGNPGMSGGGIFNANGEVIGVHQNGAQNRSGGLILSPTQL
AWIKSIIAGNEIPPVYDELYRHKDEKKDDAKDEKEVIKKLELRNISSVELYSKDGNKYRH
VTSLASLPSNAENYFMKVKSENFKDVMLPVTSITNDTKDNRDVYKIVASANSLIQHENNN
VLENYTYYLPKTQQSETGVYTSFKNLVDAMNSNPNGTFRLGATMDAREVELETGQESYVN
NVFHGILVGTNNEKYYAIYNLKKPLFGELNGATVEKLSLKDVNISAKDDTATLAKEANNN
THIDNVHADGAIAGERSIGGLVSQVNNSTISNSSYTGRITNTYKTVASYQIGGLVGKLSG
PRGLIDKSFASIDLSSNATQGDQSIGGIVGAVENSALISNSYAEGNLNNVQRFANVGGVV
GNLWDPVGGLEKSGRLSNVLSDVNVTNGNAIAGYNFNGIKANGTYSNKNNKVVNVVQEDD
EILTKDSTVQRGEVLEDAQIKEKKATFVSKNTIKTEDFNFSSRYVTDYKNLENADSSKEK
VYKNIEKLLPFYNRETIVKYGNLVETSSNLYNKELLSVVPMKDKEVISDINKNKSSINKL
LLYYADNTSETLNVNYQTDFSNVAEYRIGGTNLIYTPNTLLRNYQNILDEVLPALNSVEY
KSEAIRKVLDVSKDVSLTELYLEEQFNTTKTNLKDSLTKLLTADAAIAENNNKVIDNYVI
EKIKNNKEALLLGLTYLERWYDFKYRDTKAKDLVMYHLDFFGKSNSSALDNVIELGKSGY
NNLLAKNNVITYNVLLAKNYKTNNLFDALEKYRKAFVPDKTNNEWFKEQTKAYIVEEKST
IKEVSDKQSIAGSPYSIGVYDRLTSPSWKYPSMVLPLLTLPEKSVFIIANISTIGFGAYD
RYRSKEHPAGTDLNDYVEKKAKEAAVRFRDHYDYWYRILDDKNKEKLYRSVLVYDAFRFG
TDEKEDKDTYQATFETNHPAIKHFFGPAGNNVVHNSNGAYATGDAFYYMAYRMLDKDGAV
TYTHEMTHNSDREIYLGGYGRRNGLGPEFYAKGLLQAPDHPNDPTVTINSILKYDQSEES
TRLQVADPTQRFGSVDDLNKYMHNMFDVIYMLEILEGKAVAKLDTNQKYDLLRKIENEYK
PDPDGNSVYATNVVRRLKPEELTKLTTFNSLIEHDIITRRGYVDEATYKRNGYYTINLFS
PIYSALSSKIGTPGDLMGRRIAFELLAAKGYKDGMVPYISNQYEKEAKAQGKVITSYGKQ
IGLVTDEIVLSKVFNNQYNSWIDFKKDMYKEREDKFGKLNKVSFIDPNGSWARQQKVTID
NINRLEKMIEDAVKFDAEDEVAKLYPETNSRVLKLKKAIFKAYLDQTGDFRSSIFENKK
>orf01807
(SEQ ID NO: 1364)
MTALGLLAIGSLIVIITKDNRNKKIATFLIVGATGLVTLSTASALNLNANIHESGRDGVL
QISGYRYVGYLELDDKTVSSVSPASTVSPVEQPKVVTEKGEPEVHEKPDYTQPIGANLVE
PEVHEKLAYTEPVGTTGVDENGNLIEPPVNDIPEYTEPVGTTGVDENGNLIEPPVSDIPE
YTEPISTVSEVASEREELPSLHTDIRTETIPKTTIEESDPSKFIGDDSVRQVGEDGERQI
VTSYEELHGKKISDPVETVTILKEMKPKILVKGTKEKPKEKTAPVLTLDRTNTNVLNRSA
TLSYHLVNTDGVTINKITATIKDGNEIVKTVDLTSEQLDKQVEDLKFYKDYKIETTMTYD
RGKGEETATLEEKPLRLDLKKVEIKNIASTNLVKVNDDGTETPSDFMTEKPSDEDVKKMY
LKITSRDNKVTRLAVDKIEEVTEEGKKLYKITAEAQDLIQHTDPTKVRNKYVHYIEKPVP
KVDDVYYNFKELVDAMNADKNGTFKIGADLNATNVPTPNKQYVPGTFKGHLSSVDGKQYT
IHNIARPLFDRVENGSVKNINLGNVDINMPWADGIAPVANMVKNATVEDVKVTGNVVANN
NIAGIVNKIDSGGQLTNVAFIGNLTGVGDKGQYMAGIAGEIWRGNLAKAYVEADIVANRA
RIGGLVAKTDNGNDSMGIGKYGSIRKSVTKGTIKTKVLFETGGFINSNLPFGKLEDNISM
MRVENGEEFFGSSDLDYDGGYFTNGWLERNFVVKGVSSGKHSYKRSRDKIKEISQDEANK
RIANFGLTADKYEINEPVVNRLNRLTRREDEYKSTQDYKSERDLAYRNIEKLQPFYNKEW
IVNQGNKLAEDSNLAKKEVLSVTGMKDGQFVTDLSDIDKIMVHYADGTKEEMDVTKNTDS
KVQQVREYSVSGLGDVVYTPNMVVKNRDKLIADVKSQLSSVELISQEVRDLMSRRDKPAE
NTDERKNGYIKDLYLEESFAEVKQNLDKLVKSLVENEDHQLNGDEAAIKSLLKKVETNKA
KIMMALTYLNRYYDIKYGDISIKNIMMFKPDFYGKTPSVIDRLINIGSSEKNLKGDRTQD
AYREIIAGNTGKSNLRNFLEYNMRLFTEDKDINDWFIHSAKNVYVSEPKTTNTELKDKRH
RVFDGLDNGVHGRMILPLLTLKDAHMFLISTYNTMAYSSFEKYGKHTEEARNEFKTKIDE
VAHAQQTYLDFWSRLALPNVRDRLLKSQNMVPTPVWDNQTYNGSPVGRRGFDSKGNPIAP
IRELYGPTWRHHDRDWRMGAMASIFPNPNNDDKVLFMVTDMISPFGISAFTHETTHVNDR
MLYFGGHKHRQGTDVEAYAQGMLQTPDSSTTNGEYGALGINMAYHRPNDGNQWYNPDPDK
LKTRDDIDRYMRNYNEAMMLLDHVEADAVLPKIKGDNSKWFKKIDKEMRSKIQYNDLLGP
NQWDSIRDLKDEEKVMTLSSVNDLVDNNFMTKHGNPGNGRYRPEDFTPNSAYVNVNMMAG
IYGGNTSQGAPGSLSFKHNAFRMWGYYGYENGFISYVSNKYKAEADKNNHGLLSDKLIIN
KVSKGNFNTLEEWKRHWYGEVLAKAKKGFEAIDIDGVHISNYDELRPLFDKAVEEDLKKP
DDFSHTVALKSKVFKALLKNTDGFFNKLFKEDI
>orf01818
(SEQ ID NO: 1365)
VFHKSLNNCKRKKVCYSSLLPSCFHDWLKNLLTKSQHFSHINFIVEGEGWRSQVRFNHAL
GNNLTHWCHWNTLDFTIWCYVIRDFFHFFNLSRRFDAIVFDIFRKQGQNILLHDFTTMTG
SLDFLPSNVMFEGNSFCKWRNANHVCVFISFHVFFVDTTVCT
>orf01822
(SEQ ID NO: 1366)
VLGGRANSVTSCTTNSHWNLTFTTKHVTCFSSLVDDIVHGNNREVHEGHIDDWTKSCHGC
SCCCSRDGSFRNRTVTDTFWTKFFKHSNRSTEVSSEDTDIFSHQEHIFIATHFLRHSKDN
GVTEGHCFCFHFISFSLVCVNIFKG
>orf01823
(SEQ ID NO: 1367)
MDMFYIGHFLDIRRDTVTVVNAIENDWQVPDRSHVHCFVENTFIGRTISKEANNDFTGIL
HLLTEGCTDSDPHTTTYDTIGTKVPSIKVSDMHRSTFPFTGSSVFTKDFSHHSVEVNPFS
NSLPVSTVV
>orf01841
(SEQ ID NO: 1368)
MISVWHCNTSSCSTCDLRWVENKAIRFHMALTQRQFVELFQETINVITLTCLTVSVAVVA
CVSICSSWIAYRRYPVCS
>orf01842
(SEQ ID NO: 1369)
MISMRNDISITSILYDIRSIKDITIICSIASLRTCQGNSSIVSWSPSFTILTMFLFLSID
FLFCTDVIRVGSILKVNIVFSIYLDNISTLDLINNILIF
>orf01843
(SEQ ID NO: 1370)
LVCYLDDDLLSIDSFTLANLIRSQILRFLRRLFSIYIGNTIIFLNRSSLIQSQLVRTNT
>orf01859
(SEQ ID NO: 1371)
MDKLIIFIEKGKPFFEKLSRNIYLRAIKDGFISSMPAVLFSSIFILIAAVPNIFGFKWSD
EQLAFILKPYNYSMGILALLVAGTTAKSLTDSVNTRSMEKTNQINYMSTFLAAVVGLLIL
AADPIEGGFANGLLGTRGLLTAFLAAFITVNIYKVCIKNNVTIRLPEEVPPNIAQVFKDV
IPFALSVLSIYGLDLIVRNIFGTNVAESVGKILAPLFSATDGYIGLAIVFGAYAFFWFVG
IHGPSVVEPLIVAISYANIEANVQLVQAGMHADKILNPVTQTFVVTMGGTGATLVVPFMF
MWLCKSKRNRIVGRASVVPTFFGVNEPILFGAPIVLNPIFFIPFVTAPIINVWIMKFFVD
VLQMNSFSIILPWTTPAPIGIVMGTALAPLSFVLAITLIIIDTLIYYPFVKVYDHQILEE
ERKGNSSSELKEKVAANFNTAKADAILEKAGVDAAQNTITEETNVLVLCAGGGTSGLLAN
ALNKAAAEYNVPVKAAAGGYGAHREMLPEFNLVILAPQVASNFEDMKAETDHLGIKLAKT
EGAQYIKLTRDGKGALAFVQEQFD
>orf01861
(SEQ ID NO: 1372)
MIFSNQIPLLLSECNPLTNYNHLFSLIISDKRDIVIHWI
>orf01868
(SEQ ID NO: 1373)
MDGFIVTVKIIGHLLVVVFSAIPFFKEFCKEVCVCLLSIVTFKVFNFRNQFLVFFRWFVF
SMNESFDDITHKQFTSNLTTKADNVSVQLFFSIKGCCHITMQGRTNTWNFIYSVVDTNTS
TTDTYPKISLAASYSFPYFFTKDWVVSPCMVICTKVNDFISF
>orf01871
(SEQ ID NO: 1374)
MLDFQDRSPWLEGQKEIDLSYDLFSTDAVTLDELQSRTIALRSLKHDKGLKVHFAEFPNL
IIWSTLNKGPFITFEPWSGLSTFLEEGDHLEDHKNVCLLEANQVEELGFEIEVL
>orf01872
(SEQ ID NO: 1375)
MKLFKMSCRNIGQAGKILADSGYQGLMKIYPQAQTPRKSSKLKPLTAEDKACNHALSKGE
ARLRTSLPK
>orf01874
(SEQ ID NO: 1376)
MRRKYKSIALKKELANDSGKKKCHAMKAQAIVTSQGRIVSLDIAVNYLL
>orf01878
(SEQ ID NO: 1377)
MKIKEQTRKLAAGYSKHNFEVVDETDEVSNHTYSKATLTWFEEIFEEYKN
>orf01886
(SEQ ID NO: 1378)
LIESQVFSSLQVCCLNLCHLKFQHFDTCLVFLLVFLDFQNLLAHFPIGIKTRLIGFFQVP
KSGITKFIQHLDMQLGTH
>orf01887
(SEQ ID NO: 1379)
MVMLTMNIYKMLPNSSQNRQINHLTIYTADTTTILQDFPTDDNFIT
>orf01888
(SEQ ID NO: 1380)
MTNNICRRTSSQHHIHGINDNRLPCTRFTSQDSHPLFKIEGNSLNNGKVFYRNFK
>orf01899
(SEQ ID NO: 1381)
MPHTRDNWQTREKNSSYHNFFVKGPEILNRTTSTTNNEQIQIVPLISTRNISSNFLRSPF
TLNLGRIKKDVNTWESPADGRDNISNNGSTTAGYYPNSLRKLG
>orf01900
(SEQ ID NO: 1382)
LLEAFLKQAFFCQFFLKLFKLNRKRPNPIRLSFFNDDGVATTWFIDLYTPNHIDLHSFFQ
VKP
>orf01911
(SEQ ID NO: 1383)
MFMSNLCQFFQVWNINQGVTQGFNQDKLGIVFDSCFYFLQIINIDKGCCDTITRKGFFQK
IEGSTVNSRSSHYMVTSMGKRQNRISHCSHT
>orf01912
(SEQ ID NO: 1384)
LINVFSHGVDIAIHSATKFIGGHGTTIGGIIVDSGRFDWMASGKFPQFVDEGSSCHNLSY
TRDVGAVAFIIAVRVQLLRDTGAALSPFNAFLLLQRLETLSLRVERHVQNAETIVDFLVN
HPKVEKVNYPKLADSPYYALAEKYLPKGVGSIFTFHVKGDEEEARKVIDNLEIFSDLANA
ADAKSLVVHPATITHGQLSEKDLEAAGVTPNQIHLSIGLENVEDLIEDLRLALEKI
>orf01913
(SEQ ID NO: 1385)
MTRDFKFETLQLHAGQVVTPATKSRAVPIYQTTSFVFDDT
>orf01917
(SEQ ID NO: 1386)
MSQKNNKKKNKRKNLLTNILAGFLILLSLALIFNTQIRNIFIVWNTNKYQVSQVSKEKLE
ENQDTEGNEDFDSVKAISSEAVLTSQWDAQKLPVIGGIAIPELEMNLPIFKGLDNVNLFY
GAGTMKREQVMGEGNYSLASHHIFGVDNANKMLFSPLDNAKNGMKIYLTDKNKVYTYEIR
EVKRVTPDRVDEVDDRDGVNEITLVTCEDLAATERIIVKGDLKETKDYSQTSDEILTAFN
QPYKQFY
>orf01924
(SEQ ID NO: 1387)
MRWNIGCHPNRDTSCSINQKVWKTRWQDQGFPFIGIIVINEINCIFVDITKHFQSNLAHT
CLGITLSGSTISIHGTKIPMTIYKHVTVAPPLSHTDHGFINRGIPVWVIFTHDIPCNTSR
FFMGFVWGHTQFIHSVENATVNRF
>orf01928
(SEQ ID NO: 1388)
LKKNWFFVDYYDTTIILLALISVILVLLGFAEMIDLDNPPYSIIDLVIWGVFVIDYSWRF
FITKRKWRFILENIFDLLAILPLNAIFTVFRLGRIFRLAKLTKLLKLTRLLRIIGLTGKL
ERKISRFLRTNGLIYILYVNIFIVLVGSSILSVVEEKSFSDSLWWALVTVTTVGYGDIVP
ASIFGKWLAVLLMLVGIGTIGMLTSALTNFFVKDNPDEQIKLDKLQDELSSQRILLEKQS
KKIEELHKMIQDLIEKT
>orf01938
(SEQ ID NO: 1389)
VVDFKQTRQDPHDITIYSWLRQVKSNTGNGSCCVRSNPFQAGNSFIGIWKLATKVSHNLL
GCSLHIANSRIIAQALPSFQ
>orf01943
(SEQ ID NO: 1390)
MAERTVVQVHNAFPEDTTLINSQLIPLVQVVVNQGRKGIVGSCNSMHISSKVEVDVFHWQ
NLCIPTTSSTTLDPHDWTKRRFADSNHGFLANLVQGIRKTNGKRRLSFTCRCWVDGSNQD
QFTDWIALNCTNFIKAEFSLVLSVQLQIVVRNTKFLYNINNWLQLNTLCDFNICFHSKFL
>orf01950
(SEQ ID NO: 1391)
LRILDSQPCFFVDFTNDRLRKSLIIFYMTSRKGITRPAIVFRGAILHHHALSFEVFNQTN
IG
>orf01957
(SEQ ID NO: 1392)
MLFIIGHLNFPTAGSFIDSTLHRLGNRVCIHDDMAFTVTSSTSNSLDESTFVAKETFLVS
IENSYEAHFRNVNSFTEQVNSDQDIKDTQAQVTDNLRPFQGLDIRVHVLDLDTHFLEVVG
QILCHFLGQSCDKGTLIFFNAGIDFTQEVINLSHSRTDFHLWIQESRWTNDLLNHCLGLF
IFIVTRCR
>orf01958
(SEQ ID NO: 1393)
MNVTLKLLPTERTIVQSRRQTETIINQHFFTRTVSIVHALDLPYGHMTLVNHNQEIIWEE
VEKRIRRLSFAPSIHVARIIFNPIGIAHLTQHFDIILCPLFQTLGFKQFTFLFKDS
>orf01959
(SEQ ID NO: 1394)
MIHFSQHLTCQSLNFTNTVNFVSKKFYSKGMFISGSWENLYHIPTNAKSSALEINIITFK
LNIDQVIQEFITRNL
>orf01960
(SEQ ID NO: 1395)
VAKLVNLVIDRTILLNIGIARRDIGLWLVIIIVGYEILNCIFREKFLKLPIELTSQSFIV
GNNQSWFIDFRNDLTHSIGLPCSSRPHQNLSFFSPLNVIHQLLDSLGLIS
>orf01979
(SEQ ID NO: 1396)
MNITKTNFLAVNFVFTIPTTIDMAFYSDFLTCILDKSIMIIQSHNYRSIIKRFTTFCSSK
DDIRHLAPTETLDTRLSQGPSQTFCNIRLSRSIGSNDCRHTLVKDDLGLISKRLESLNFD
FL
>orf01981
(SEQ ID NO: 1397)
MGFIVCNHLKFACFNLRNHDLIDKFLDLGHILVQKKGTKKGFKGITKNGITIAPTRFFFP
LTQLDKLVKLAITRKASQTLLTDNHSTEF
>orf01989
(SEQ ID NO: 1398)
MRITDNQHKIAKEDFVAEYPKLSQALLDRTLDNLSREDNIFIFPNDLTHTPDLDKDQKIF
ETVNQKIKTGNVIGFLGYGQERLTISSRFSDESNDHFLHYLLNKVLHINLTSLDVALSRE
ERLYQLLMYLFPKYLQAAIRKGLYKEYHRFSHNDSHVKGVIDVRNHLKKNLPFTGNIAYT
TREFTYDNPLMQLVRHTIECIKNQKSIGQGVLDNLSTSRENVSEIVRVTPSYKLADRAKI
IRMNKIKLIRHAYFREYRKLQELCLVILSREKHGLGPQAQRVHGILFDVAWLWEEYVYTL
LPKGFVHPRNKDKTDGISVFSVGKRKVYPDFYDRERKIVLDAKYKKLELTEKGINREDLF
QLISYSYILKAEKAGLVFPSKDKVIDNEIGNLAGYGLFESLRMPHSIVHFVK
>orf01995
(SEQ ID NO: 1399)
LDEDILLGCILPWKPEAFEKLKAYGNGREELMTDVRGTSCFVIKFGKAGEQLAAKLWEEG
KMVYASSASMTKRLKLAMSKV
>orf02000
(SEQ ID NO: 1400)
MAKKIVALVGDGIGPEIMEAGLEVLEALAEKTGFDYEIDRRPFGGADIDAAGPPLPDETL
KASREADAILLAAIGSPQYDGAAVRPEQGLMALRKELNLYANIRPVKIFDSLKYLSPLKP
ERISGVDFVVVRELTGEIYFGDHILEERKARDINDYSYEEVERIIRKAFEIARNRRKIVT
SIDKQNVLATSKLWRKVAEEVAQDFPDVTLEHQLVDSAAMLMITNPAKFDVIVTENLFGD
ILSDESSVLSGTLEVMPSASHSENGPSLYEPIHGSAPDIAGQGIANPTSMILSVAMMLRD
SFGRYEDAERIKHAVETSLAAGILTRDIGGQASTKEMTEAIIARL
>orf02004
(SEQ ID NO: 1401)
LANIESHCNFFQSSIFSSLPNTIDSPFNTSCTILDSSKAICHCHSEVIMTVRRTDDLTIR
LDILNQVFEDGTIFL
>orf02011
(SEQ ID NO: 1402)
MTAIWEIATSVEFTKTTKFNDHWTATHFTVKSSWFILNLDFFHFFFSLGNFF
>orf02016
(SEQ ID NO: 1403)
MPRNRFSFTVRVTREKNFISFFSFFFQVIDKRAFSSDINILRFIIIFNIDGHTGFLQITD
MPDTG
>orf02020
(SEQ ID NO: 1404)
MKIKAQTRKLATGCSKHCFEVVDKTDEVSSKYCFEVADGS
>orf02029
(SEQ ID NO: 1405)
VHAHTDKLCNGCNRIFNSIISHHTIFRERNKLSHKAIKSTRQEMGPCHVVFIEFFITLHR
RLIGNHDNFLTNLVGSGRVRNDGST
>orf02030
(SEQ ID NO: 1406)
VNHCHWKLFIQNLGITFSLIVTLIRMTDSHVVGTDKDMILLVNSLFLIFDIDKLRLS
>orf02032
(SEQ ID NO: 1407)
VGNNDILWSKRTISINGFNDFLNTCIAVSTTLCNDDTFLIKRKIFIYKIFCMRNPVSMNT
NYNFFNTWLQDKFFNCMNQNRSIT
>orf02042
(SEQ ID NO: 1408)
LVAPVASSTRFFKNNDSLTSWNNGFIIITINTIISYQRISKGQDLSIIRLVCNGFLVAGH
PCIKDDFACYINICSEGLAFKNCAIF
>orf02044
(SEQ ID NO: 1409)
VVCYFYITIDWSWVHEDCCFFQTIVTFLSQAMLGMVVFF
>orf02045
(SEQ ID NO: 1410)
MAFVLHTEKHHDINLINDFINGYKLSIVCKLLTSPFLRSSEKEFSSQAFQNLHIGFGNA
>orf02046
(SEQ ID NO: 1411)
VIQVTCNSNFKTLKVAKFLINGHQIKQALARVLARTISTIDDGSRNRWTSNQFSIVVDLW
MANHTDIHS
>orf02047
(SEQ ID NO: 1412)
MCPCRILKEEIGNNRMVFIGKLGSIFKLNSSLDQFHYLIDSEVFHGHHMVQCLLIF
>orf02059
(SEQ ID NO: 1413)
MQEHYTPKGKHLTIDNRRLIERWKNENKSNREIAGLLGKAPQTIHTEVKRGTTLQQVRKG
LYKKVYSADYAQTVYQFNRKRSVKKLILTKEIREKILHYHKQKFSPEMMVNKKQVKVGIS
TIYYWFHNGHLGLTKADMLYPRKRKGVKKQASPNFKPAGKSIEERPDVINLRLENGHYEI
DTVLLTKIKNYCLLVLTDRRSRHQIIRLIPNKTAESVNQALTLLLGEHHILSITADNGSE
FKRLSEVFPEEHIYYAHAYSSWERGSNENHNRLIRRWLPKGTKKTTPKEVAFIENWINNY
PKKCLDYKSPSEFLLGG
>orf02076
(SEQ ID NO: 1414)
VQFHLIIFQNLFCSLDIVIDSLTTDTELLGNFSKAVIISVVELDIIHLLICQKRRIKFKE
RIHTIGFFDFHNFYYTKN
>orf02079
(SEQ ID NO: 1415)
MKFNHYFFLFLIIEKQVAIISFFMHFHIIKLVNHFQLLIKLNCISHPNLHIRPSFLSLVL
LFYQKEQDFAIMVI
>orf02085
(SEQ ID NO: 1416)
MKKEQFYPLGIFLAAMLGGLVRYLVSTWLPASPDFPWGTLFVNYLGIFCLIYLVKGYLVY
KGTSKGLILALGTGFCGGLTTFSSLMLDTVKLLDTGRYPSLKPELAFEYRWRPAFSLLFG
EEEMVIVYLAIACGLGALVRYFFSRYNQASKLPLGTLIANLLGCFLIGVFYNHVESKEVY
AILATGFCGGLTTFSTLNDELQRLLSDKKVFYSYLTLTYIGGLVAIFLGILL
>orf02097
(SEQ ID NO: 1417)
LNRSILDNITLKHEVTSQKIEEVCKAVQIYDEIMAMPMKFNTIISEMGSNISGGQRQRIA
LARALINNPSIVILDEATSALDTINEERITKYIQSQGCTQIIVAHRLSTIKDADVIFVMK
GGKIVESGNHKYLITLGGEYYSLYTKRK
>orf02100
(SEQ ID NO: 1418)
MSLLETAKRHQLNSEKYLSYLLECLPNEETLVNKEVLEAYLPWTKVVQEKCK
>orf02101
(SEQ ID NO: 1419)
LKRPPKQADKSSLGAKGLAYCDQLFSLERDWEALPADERLQKRQEHLQPLMEDFFA
>orf02102
(SEQ ID NO: 1420)
VISIEMRTFFLYSSAFKKHSSPSPINDGLYHLLLQSLYNILELIHDIFQSLKGFILKSTF
TNLFPHLFNGVHLWCVWRNKCKANISRNL
>orf02129
(SEQ ID NO: 1421)
METKKIKNLKGQIIVSCQALEGEPLYTPNGGVMPLLAKAAFQAGAKGIRANSVRDISEIK
EEVDLPIIGIIKRDYDGFEPFISATMKEIDELVSEGVDILALDCTNRSRPGYDNITDFIH
DIKVKYPNQLLMADISTFEEGKVAAESGVDFVGTTLSGYTPYSPKKDNPDFELVERLVKE
LDVPVIAEGRISTPEQARKMLDLGAYAVVVGGAITRPKEIAQRFINVIK
>orf02134
(SEQ ID NO: 1422)
MTKDILELESQKMSSDTFIDEIKNNYLSIVESTRKLIDGRQIELAIKLIREANQILMIGV
GSSGNAAREFESSLLRIGIISKTVIDTHFQLMHTALLKDNDLIIAFSLSGSTKEVEETLL
NAKRKNVKIISITNYSSRNIAKLSDCVLLTSKKESYLEGGSLMAKASQLFIIDVICTRLS
LINYEDTICKKEEIASLLSNKVE
>orf02135
(SEQ ID NO: 1423)
MQIKFIDKVSNLIMLNLLYVASVVTVIAIGSGESALIATLIKIVRHEESYPYRDFANSFF
KDYWKNLGAALISNLPILILLFSLFFLPYIPLPIYIISILRHIGVIYIILHLIATTFLIP
LIGRYNNTLKNSLHNSIMLAYKHFFIAVLIRIIEIIPVLLFFILQNQLLVWITLMIFILP
SITKYANAFLYNFIFSKYEKLN
>orf02136
(SEQ ID NO: 1424)
MVSGGFRLDFLLETARLARSTYYYQLKQLDGVDKDKEIKTEIQGIDNEHKGNYGYRRIHL
ELRNRGFVVNHKKVQRLMRILGLTARIRRKRKYSSYQGEIGKKAENLIQRQFEASRPMEK
CYTDVTEFAIPNSTQKLYLSPVLDGFNSEIIAYHLSTSPNLEQVKSMLEQAFTEKYYENT
ILHSDQGWQYQHDSYHRFLESKEIQASMSRKGNSPDNGMMESFFGILKSEMFYGYEKNFR
SLENLEQAIVDYIDYYNNKRIKVKLKGLSSVQYRTKSFG
>orf02137
(SEQ ID NO: 1425)
MKLSYEDKVQIYELRKQGQSFKQLSKRFGVDVSGLKYMVKLIDRYGIEIVKKGKNRHYSS
KLKQEMMDKALLEGCSQRSISLDYALPNQGMLSFWPAQYKKNGYTIVEKTRGRPAKMGRK
RKKTWEEMTELERLQEENERLRTEVAYLKKLKELEERDEALERERQRQLEKWFQEDFD
>orf02152
(SEQ ID NO: 1426)
MVISKTKKYKGVYKDSKGKIYFQIELGVDPITGKRIQKKGRKNQQGLPFNSFKEAYEEIL
RLKHEFVNSTINNSFLTFREFMEEIYLKYYQQKVQFVTYQTALPHHQLFIKQFGSKKLSD
ISTIDCERFRLAIIDKYSSNYAKNMWSRFKACLGYAERLGYIDRVPFKGLDNPRGKHPDT
KFWTFDEFKKIINSFDISEYEGLHNYMTIWLYFMTGLRVSEGIALKWEDIDFERKWIHVH
STIEKDKNGVWYAKQQTKTVAGNRKIDLDDFTITILKKWREVQIKNDDKDYVISRFGAPL
CKSTISRIIKRHAKITGVPEITGKGLRHSHASYLINVLHKDTLYVSYRLGHADKSTTLNT
YSHWYYSGDSTISEEITNSLDNLGLSIYLPNSCQS
>orf02153
(SEQ ID NO: 1427)
METVNYKDLVAIGFPEHTSRNIIRQAKKIAVKKFEEARKNDKNAVQLGCSPFDNKRLGIA
PKNIVENLIGISFSDIEGEKNGYIKDKEI
>orf02154
(SEQ ID NO: 1428)
MLKRIRDLREDDDLTQEYIAKIVLNCTRSSYSKMEAGSRLISINDLIKLADFYKVSLDYL
VGRVDNKEDHYSKK
>orf02155
(SEQ ID NO: 1429)
MVITKHFAIHGKNYRSKLVKYILNPSKTKNLALVSDFGMRNYLDFPSYKELVKMYNDNFL
SNDGLYEFRHDRQEVNQRRIYSHHIIQSFSPDDHLTPEQINRIGYETVKELTGGRFRFIV
ATHVDKGHIHNHIILNSIDQNSDKKFLWNYKSERNLRMVSDRLSKIAGAKIIENRYSHRQ
YEVYRKTNYKYEIKQRVYFLIENSKNFEDFRKKAKALHLIIDFRHKHVTFFMTDSNMKQV
VRDDKLNRKQPYNETYFKQKFVQREIINILEFLLPKMKNMNELIQQAEFFDLKIIPKEKH
VLFEFNGIKLSEQELGKMNQYSVSYFQDYFNNKNETFVLDNNNLIELYNKEKLIKEKELP
TEEVVWKSYQDFKRNRDAVHELEVELNLNQIEAVVDDGIYIKVQFGIRQEGLIFVPNIQI
NMEEEKVKVFLRETSSYYVYHKDSADKNRFMKGKTLIRQFTLQHEPQHMYRRIPLSKIKE
KIEQLDFLISAENSPNDFEDITNDFIAQISYLENMIEQVQNKIDDLTNLEEVLLNNTTNS
SSNLENSIQGKSSVDTIEKDLYIYKGKIETLKEQHGEAINLFEMFNKTIKKYKKKQNMKS
IEENEIHLE
>orf02156
(SEQ ID NO: 1430)
MKRDIRSIRKQFRLTETEEKQILDLMREKGEDNFSDFLRKSLLLSDGQKQMEKWFNLWKK
QKLEQISRDVHEILIIAKINHQVTQEHVSILLTCIQELIKEVEKTSPLSENFRNKYMR
>orf02157
(SEQ ID NO: 1431)
MEYVEAVNQFIERHYKEKDIGHIEIDFWGNKNHPHSLYIYKRSKKIEYDYFFFDSIDYYE
EPDFLEFKYIVHLENITYIFWQED
>orf02162
(SEQ ID NO: 1432)
MTTLDFKTLFKEEYDKLNKQQKKAVDTVEGPVMVIAGPGTGKTQILSRRVANILTNYHTS
SEEIVCLTYTEAGASEMLDRLEKLIGEEGRKVRVSTIHAFCSELILRNSEIFGGQPKIIS
TAAKYEILKEIMDEYVIEGNPLYKNSGKRYSAKDQLLELFYKMKRENLNKEDFEKEIDEY
FKMIDLSIPGDDLYSKFKYARNSKSKDKKVGDYKDKAINELKENTQKLLAGVEIIEKYSS
DISNHNYFDFDDMILWTIEKLEENEGFQRSVSDTIRYLFVDEFQDTSVVQNKLVDLLVKG
KDNPNIFVVGDDDQSIYRFQGVSANNIRDFDKKYKPTKIVLDENYRSSQAIIDASRQLIS
HNPREEKLLIAAGANKDYDYQLPILKSYENAKAEMFGVLTEIKELIDSGVSPNEIGVIYG
RNSYGEEFAKILRDKGIFVQMKENKDLFSEPFFKKIVAILKYLCKPSRDVRELRKIVYFD
FFEVVLSQIVMIRNLKKDEKISIPTIAEIDQKLEIIRKKVNQSSKYLSPMYVLSDVLKSL
SIDEYIMKSKEKYHLVSVLNELYKLMLMECHIHPKLTVKGFLNQLSALEEMGISLPIEDI
SGSPSNCVQLMTAHGSKGLEFDHVFIMKCNDGKKKSEAWPGGENNSGRFSYPPSLNGKDE
NESQLKEEENRRLFYVAMTRAKKVLHLSYANDSTKTHLINEFEEFIDEVDVTESFEDCQS
VDKVVMPKFSNNVINEIFDELSLSVSTLNSFLKCPLSFYFNKGLKLPSETNEAMVFGSII
HEVLEKIYISVDGSQSSELTAKTVLSLEEALKLFETVFEEKSYQLTSNKIKKDDYARGKK
IIENLYKKSGYLKDGVVAVEVPIQGIRLGDILNTTVDLSEVSNIEINGKIDKIECDGNIV
CLVDYKTGNFENAKKKLVAPSEKEPLGGDYWRQAVFYYILFKNAGIDISDKEILVKYVLV
ENSTNEDGFSETEDIRITQKEVDIVLNQIKESIMKIKQGDFNCGCGVLKKDRDNYPCDYC
LQVSANTTPKFDNTEALEVATYQQTRGNYKSLSVSKLNRYLRCPKSIYFEDVLQLSQAAG
LSAGAKEKSTKITINHAPTGPVFGTAIHETMEKIYKEDLQLEDAIEFYDSSLYSHQEEII
DTMSVEELKEYGHNLLTNLFEHFIPNSLKGEHVSLEKELRVKLGDNYSINGIIDKLEFDN
DLIRVVDYKTGSAQRGVEELEVGHDYWRQAVFYNLLLENSSEIDTTDKRIETQYIFLDDN
STESGYSIHTIQVTKEDLDLVTSQIQNFWSHMNTADFTGSCGKNDCDYCRLAEFVDFELL
KETIESGKESNLVN
>orf02163
(SEQ ID NO: 1433)
MSGRLTRQNYYLLGKLIDEFHAVKAAMRVIETKRNDFNI
>orf02164
(SEQ ID NO: 1434)
MKPQERLLTIFFRLQAGERLSKAQLSDEYEIDYRTVQRYMSTLKNFLQEQRISNTEIKFD
TSDNTYRLIAKTTFNKKDILVISKILLENRALNKSELYSLLEDLLSLLSSEEQKEIDAII
GSERFNYKSLTNDKDRIDTIWILSEAIRREQMLEIEYKAPLKDIKSHIIFPVSLYYDAHY
FYLVAYHLKHENYTTYRVDRMESLSESHVKKPEISYGRKYRDGEVRNQKVDAFEGRKIDV
TLIYKGNTEIVLDQFPEREILSENHDEIKVKIKTQDTPGLKRWILGQGDAVTLLSPSKLI
EEIQESLENTLRNYKK
>orf02165
(SEQ ID NO: 1435)
MSVQKTKNTLNEPLKTLLDEYHDKVGKINNSSELFDLYSPWNNFNIEKMIESFNKALQSN
SNNFSWLDIEEDLPKSTDVDIKYGLPNHIKGNIDEATLFLCLVNPNIDEVKTEKKDVGIH
TYYKKAREMESGDDSLNILNDKGKLRIDPKVYIKEHILDVRETSSILYNELQIVKQTRSY
KDTYYLGHYLPHFIKEFLNKKGSFKNVIHNLTDEWDELEKMSKKIANLEAFPFRSQNPNY
TYKSNKRATNFTNLLIESDSKVNLLSARVIIWRIVKHLESSQHKPAFILRRFNTFWLPTI
SKVLEQDLNFTKEEINQIINALDEEYFFTVRKKDYNGQSGYFGRNFCKNNERISNSSFKH
LVQETLGEYVKK
>orf02166
(SEQ ID NO: 1436)
MSGSFSDSPTHDDKFSIENYINGLSNFIIECETPLTVAIQGDWGTGKTSIMYQVEKRLNP
EKQDKKIQTIFFNTWQYSQFDMGNNLAVALITDLISELNVEDSKKKQFFKKAKGALSKGL
EYVNLDFGILNGEKLTEKFQDLIIGFGERTDDIKHLKENLQDIINDAIKENKSDRIVIFI
DDLDRLVPEKAIELLEVLKLFLDCEHCVFVLAIDYNVVVRGAKSKYGKDLDDEKGKAFFE
KIIQVPFTVPVANYDLQNFIESSLKKLDFCFDKNNKERNQLETITQLIRYSIGNNPRSIN
RLFNSVSLLMYINNGDKVDHDEKLMILAMVCFQLRFEEAYNYLLTAYNNSPEDSDDIESY
LIDLLENSFELLDDEVYYNSLVSLLGKFTFKDKKDRDDFTNFYRTLKELLGYNEQGLTME
QFNKLIEKMTFSNAVSIGNTDTITADKKKQNHAPNEDVQFVIRKLFNTLVGDENYFDLKK
PELFGKETREKREAPLSEEFISIPNEFDRIRLTRGKGQGLNIYSSHNKSNFIYISGDTHG
RMLNDGMAIVVNNNLVEKIKDNILASDLRSEELYHEFEMNFRDNLNKLLSKASKILNN
>orf02167
(SEQ ID NO: 1437)
MEFIRAANQFIENYYPREDLDRIESIEIGIRDSENYSRYFLEIQKQSEEFECDFFNFDNI
DYYVVDDSVHFKQIINLENSSYVFWKDY
>orf02168
(SEQ ID NO: 1438)
MNKPIAAIFDIDGTIFRDSLLLKHMEKCVSYDVFPNSVNSEIKFHKNAWENRELDYDDYL
YIAATLYTKYIADKDILDIDFVAKKVIEKESKKLYRYTRDRIKWHKEQGHQIIFISGSPD
FLVSKMAEKLGADIWYASNYLQLDSKYTGEVIPMWDSTSKLQVLKKLFIDFEKSYAYGDT
TGDFTMLQSVGFPTAINPNKKLLDKITMEKLDCKIIIERKDVIYKLDEVTHGIY
>orf02169
(SEQ ID NO: 1439)
MTNAKEFALTAHKNQTRKGKITPYSFHLFLVNNILETLTEDPHIIATGWLHDTVEDTDVS
LEDIKQEFNDEIYSYMSLESEDKSIKDWQTRKELQLAKFREAAEDESLRKVLLVTFSDKL
ANLMELYQDYLIIGGLLWDRFNSKDPKKQRWYFNEFYKIFKDNQDLFSKNKDILNNYKEI
LKLLFYNN
>orf02170
(SEQ ID NO: 1440)
LKAIVKNPKRLFELLRLYFVPVKGRKVVHVPAYAYKEDENEKIYLHNNELHLSKKMFEFL
VNQGLDLVECLPEE
>orf02172
(SEQ ID NO: 1441)
MANSSEAHGRVYIKASNLKTIEDFLLIQEERNKYVYYPTDIIDSQSNISDIVSSRTTQEN
GYYICNMWFTAEGRWCFENNIDDFFDCTLFQDTDDVLIRQMKEYVCSQDIQIKFEYVDAE
ASQNFVKEQEATITYNSKTKDISIDVKTIKDLPYTAENLIVYGYYECDEIVSVQFLLDYY
DDYLRGNEFYLKHKDGIVPILERQQ
>orf02173
(SEQ ID NO: 1442)
MAESALINLINFSKENEELTNLVSGHASKREKATISKDGLIQARSIENFIDNYALSDFDF
STIKEKCVFIKINNSFQADDTTEDIYHNVRGVWNISESRKKDLEYALALYRGVCVGVYKI
QGWKKAYEHSSEYPFPTRKEGGKIETSEETIVKYSNIEDLKKDYPELYKRSFSNSEFPQK
SLDKWRNRSFFYGNWDGSDVPQHLAQCLNKRIINIPKFTKSVKEFKSIDNQASVIYNDLK
>orf02174
(SEQ ID NO: 1443)
MDLVEDCNTFLSFVADKTLEKQKLYKANSCKNRFCPVCAWRKARKDALGLSLMMQYIKQQ
EKKEFIFLTLTTPNVMSDELENEIKRYNNSFRKLIKRKKVGSVIKGYVRKLEITYNKKRD
DYNPHFHVLIAVNKSYFTDKRYYISQQEWLDLWRDVTGISEITQVQVQKIRQNNNKELYE
MAKYSGKDSDYLINQKVFDAFYKSLKGKQVLVYSGLEKEAKKKLKNGDLDYLKEIDPTEY
IYQIFYIWKQKEYLASELYDLTEQEKREINHKMIDEIEEEQ
>orf02175
(SEQ ID NO: 1444)
MNFNKIDLDNWKRKEIFNHYLNQQTTFSITTEIDISVLYRNIKQEGYKFYPAFIFLVTRV
INSNTAFRTGYNSDGELGYWDKLEPLYTIFDGVSKTFSGIWTPVKNDFKEFYDLYLSDVE
KYNGSGKLFPKTPIPENAFSLSIIPWTSFTGFNLNINNNSNYLLPIITAGKFINKGNSIY
LPLSLQVHHSVCDGYHAGLFMNSIQELSDRPNDWLL
>orf02176
(SEQ ID NO: 1445)
MNYQKLNDITGATKNEKDKYYVYGLYEEGKQLPFYIGKGEGTRLISHIDEALTEINQEEN
IQISKKIQIIRKHKGKIIPVII
>orf02177
(SEQ ID NO: 1446)
MTTTKNPWNQLSNVDINGEQAILATEDVELIKKYTSTKHYKNLKDDIYRLQLGFCPQQFV
GDIQNADIIVLSKNPGYTPEFKTLYDHDKNYQKTLLNNLQLKGNLYFHAFDLDTNEFGYW
AKKFKVWFDDVDNLQDLKEKLPWFSKHVALAEYFPYYSTKYDDKLNDFISKEGYFPTQKF
LENLIRERVLDDNDPVIIIITRSYNKWYDAIPQLKEYKNCYETSNPSNPSLKPENLLKVK
RYSAKKEVEKVLEDSLKKLEHK
>orf02181
(SEQ ID NO: 1447)
MFTKLFKKNQDNSDVFKKLIHRLSDMSIQDLKKIDRLLDIIFTPDQGSEQLKTEATYREE
TLDDTLKEAKNQLHMEQLEKNLERFRKNSQ
>orf02183
(SEQ ID NO: 1448)
MTKDWNFNQPLESKSENQEDPDKIAALFGNHQGGNEVNYEAAFQKRKQAPVTESNSSSKP
KVTEVRTGKETDITTSYQQHLKRLIADNNSDIQSSQKKIEELHTLIDTKNKDNKKLQSIY
DAISELH
>orf02185
(SEQ ID NO: 1449)
MTIIERLEEKVTRQESKVARETEKLAAYKEQLETAMFATFKRRQSISHMSFEEALDHAFG
KERQFDDSEFRKDEMSE
>orf02187
(SEQ ID NO: 1450)
MEIEECKKISILDVANRLGISFKQVSSSVYEHPEHDSFRIFSTTNTFKWFSRDIQGDVID
FVRLVKGISFKEALAFLSEEPFQKEAVQEKRERPFYYPLKRIEDSNCSLARYYLTECRGI
SEEIIQKMIQQGLMSQASWKTNETVEPVIVFKSFDHRHKLQAASLQGIYKNHSLPRERLK
TILKGSHGHVGISFDIGKPNRLVFCESFIDLMSYYELHQQNLFDVRLVSMEGLKRSVVAY
QTLRLIAEENQKLEFLDTVIPSKLLPLINTIRDTTSYFDNHPDLLTLAVDCDDAGKDFSD
KLSRSGFPVFLDLPDNESGKEKRDWNDILREKKSDLQLMIENAKETLRNQPVRQTSQCLE
L
>orf02189
(SEQ ID NO: 1451)
MLNKVKTKALISVGAVAATSFILMVGYTIGQHSTAKQSRKEIELAATKLVEDKQAEDKAS
ILSSDTVKEFLTQYYTKEKLGENNTRIQPYMTESAYSQELSSQNDAMNQVYKDYILDYHF
EKADIFVNQTTNQAIAMVSYNVTYVSDLKNANQSKTNQTETRTVKLSYSKLPGKLLVNQV
QVWKSGLDDLDKVTPKTLEESSSIPSLPNTTTK
>orf02190
(SEQ ID NO: 1452)
LKKVKFIQQLSETSCGLACMAMILDYYGHEANLYELCCDFENSRDGLSIREIKDIASYFG
LDSKATEILNIKKFLGNKFVEPYIALTQNAHYIVVEKHNEHSVVFIDPERGRITEDISNF
ANNISGIVIFFSPNRKFTKKKKHSNFFKILKIGKIDIKRLCYISLISILVQSLTLLLPLL
TRFIIDNVISKGEMYRLGMLFSIFSLITFYALFSFIRTKLIITVEKRYIFTLKDKIVGKI
FTLPMKFFDSRSSGEIVTRINNLDSLEKIISSGISSLLIDLSTIIIAFIAMAMISLYFTL
IITCFAIFLFIVLYFLLKKLEEKNSNFISSKELTQGYLMEIFSNLLFLKVSAKGDVSYTK
WKEMFSNELKFDVERENYLNIFQTFISIYRLVPNLLILILGGLEVQQHMMSLGSLMSFLS
LVSLLLSPITLIVQNCFQFQFCFTILDRVFDIIYTPEEKNQFSISKLPPFEMMTFRNLSF
AYSSACKVIYDISLTIKKGERIAIVGRTGCGKTTLIKLLLRLYDVEKNTILYNGNDINLF
DLNSYRKSFGVVLQNDVMFNDTVISNIDLTHSHSMEDVISAASLAELDIEINNMPMGYYT
SIGDNGNNLSGGQRQRLAIARAILQNPEIIIFDEGTGQLDTITEKKIMDNLKHKGITQIF
ITHRLSNAQEYDNIIVMDNGKIIDSGKHDELCGKEGMYKRLFETSYN
>orf02191
(SEQ ID NO: 1453)
MNRKLNITDFKDASYFSERKISSHDLGDGLKSRKRKEYWDNFIGEGSNEFWKYLQEKNDF
SKHDLDIFLEEHTYDVKDIDSFSHMDEFIEFFFKRNDYTLPEFYIVDSEGKRKPIFSNFV
IPFLKFAAYKLEDNLSSQNLKVTRDVLNSLLIALFQQILNISYRTLILELQVLKEQNMLK
GETGEKRFKYFSEIYLSDHFWDILKEYPVMFRLIIENIQNWVTNNVEFLTNLKEDKALLQ
EHFSINGELTKIESGVSDFHNHGKSVYLLWFGTNKLVYKPRDLILDVKFQNLLSWYNLKF
NKNLYVTNILNRGNYGWVEYIEHLPCTYESDFIQFYTHLGYLLFLLFAMRGNDIHFENII
AKGNRPVLVDIETLFHNTTEYRKEYETADKLIFSLLEKSVKRVGILPNIVWGKDGNSGVD
ISGLSSSAGEMIPIERASIMHSMTDEMKIGYEQSALQSKDNQPFIQSGKDVDLNSYKNYV
SAGFKEAYEIISKDPSSIEEFLVEIEKFNNAYSRQIMRPTQFYSNLIQTSYHPSFLRSGL
DREMLFSKVWKIVFEDKKVQRIASSEFESLLLGDIPLFQTKISDRFLCSELTKYQNFFNI
SGMELAIQQIKDFCQKDMEFQLNLIETTLNYDPSYNLVQDSFVPLKPSVKVIRNLNQKEI
QETKSRIVPLTEKIADYLSKISYSGTSGDICWVDMNILGEKTNDWNMVPIGCDLYNGISG
IMLFYLFLYLETKKKDYLIYLKKCYRSLKYYLDSRKQFATHSQVLFGGFSGETPIIYVLL
LLKTRMPEHFNSDELDVYIYDIIDDLKKGYRYDENFDVLVGSAGAIIHLLNVFEVTRDEE
LLILAHDLFSHLEKNSTKITVEGQDGRAWKGTMASNPLAGFAHGVSGIVWALSKLSRYFP
EDKKLKTIIKQGIIFENSMFDTEKSNWSDYRETESGIKYKDIVENIPVSWCHGAPGILIS
RLELYKNNTLNVEFRRTMKSDMDVAIDTTIKYGFGKSHCLCHGDLGNLNILFYVAKKMSS
EHLYNVVYSYLNTILDDLESENWKCGLPYKNSPSLMSGIAGIGLGLLTLNNLSIPSVINL
EIW
>orf02192
(SEQ ID NO: 1454)
MLMLEFTKLRRRKILYMIPFVAILLFLLEFMIGHQIYQGHSYGSVNGWYVENGFFFLNYY
FLLPFASMILVDLIRIEQVSKTISNLRLIPVDLKQLLQAKFMLALLINLLVSEFTFLAML
VLELMDGDFAFSSLAMLSWGVVYGAIAFAYTLSAGIIVLFLGKYRKELILALPLAFLLSF
AGLFALTTVVGRYYLANLLLIIMEQFTALTVSVYAIWLVTLVACLLYLLADKRMMNIIFA
YK
>orf02194
(SEQ ID NO: 1455)
LLDLVKIEFLKQRHQKLNFVVYGVVSLYLALICYYVNDARGLFDSFPFVYKFSLSYLNFL
ILPLYCVSYTIQAFGLEYRYRIMNNLKLASANLMKTFWAKILYIEINALCIMLFTYISVS
LFALLSRFSSTVSLSLLLRFFYLCMSSGILIPMGVFPLVALVMIKVRGKEIVGNLVGVMY
VLVSFFLARTSPNISPVTSANSLIWEGNREGVVLQQPAILSIVVLGLSLLVLSFLSIKSW
LRKVDE
>orf02195
(SEQ ID NO: 1456)
MRNPVIQTFNLSKSYDGKIVLDRIDFTLRQGEIYGLLGRNGTGKTTFIKAILGLTAMDSG
EVNILSEKLLGEFSKDLLSQIGVVLDSASFYPNLTGPENLSIFARLRGISLKQVEQALQV
VGLDGENKKLFKQYSLGMKQRLAIANAIMHQPKILILDEPTNGLDPIGILEMRRYLKELS
TNHGISILISSHIISELEKLVDRVGILHDAHLMAEKTMKELIDGADKRKIHLIVSDAPQA
KEVLCRINLQEQISILSDIELELQGESPTFDIAVVSNSLKDNGIVLKEYSYKNNESLEDY
FKRITGGEGIA
>orf02196
(SEQ ID NO: 1457)
MTILSDKLKAKRKEKGFSQKTLSEGICEQSQISKIERGNYMPAADLLYKLANRLQVPLDY
FFDEQIEMTSNITPFKKLAEKLLEDRNYEDLEYLLNLEKEKSQYLSTEDEFYLLWIQSII
LFYLHSSKDEAIASLENALPKLSVSSSVYLKLLNTLSNFYFSVGRDAEYEENNSLLISLY
QEKDLNHQEYLFGYIRVKHNFAYYLHSKGKELEAVQEALETIDFCKQKETSYQLAPLLTI
VANAGKDFLKHDEILDYYLQARDICKIYEHKLMMAKIDHFLKDKDR
>orf02197
(SEQ ID NO: 1458)
MNDLLLIPVIFLAVGGILILLWRLFLIASGLFLIGFVSFLIFVEVYGIYLLFTETELYTA
DLAQNGLFGFTTFFIIFNLVLLALACWAGYKWKRGY
>orf02199
(SEQ ID NO: 1459)
MEDTYYQLEEALVQGFQTPEEYQAYKELKEHYEEVTGDYSFSKRELTSQLEIALQNHRGV
DFEEYEKKDYLELVQKLEEFDSSLATHYRQLID
>orf02200
(SEQ ID NO: 1460)
MVRRWVLSLQRNGRIIRQGNENKEVDIYHYITKGSFDNYLWATQENKLRYIKQIMTSKEP
IRAAEDIDEQTMTASDFKALATGNPYLKYKMELENDLTLLENQRRAFQRSKDHYRHTISY
CEENMPILEKRLSKYEGDIQQSEISKDQAFSMRVGKQSFDQRAEAGESLHRLIRHNQADS
KEFRTLASYRGFDIKMLSLPTNQPLPETFSVKIVGENQYSVSLDLYSPLGTIQRLQHTID
HIKEDQVKTQNLLDELKDKWNTAKVEIEKNFPKEEDYQTKKAEYDVLAPLIETETDLDTI
DQALRQFHEKGKEKQEQLSFELD
>orf02202
(SEQ ID NO: 1461)
MRNPQNVLNNLTKHSKDKNYQFERLYRLLYNKEMYLVAYQTIYANPGHLTPGVDELTIDG
MSIARIDQLIDSLKDESYQPHPSRRTYIPKKNGKLRPLGIPSFDDKLLQQVIKMILEAIY
EGQFEPSSHGFRPNKSCHTALTQIQKTYTGTKWFIEGDIKSFFDNINHDVMIHILRERIT
DERFLRLIRKFLNAGYVEDWKFYKTYSGTPQGGIISPILANIYLDKFDKYMTDYVKNFCQ
GKYRKRTPEYRQNEIALGKARRALECVSTENQRQEVIQRIRQLEKERVLIPHSDPMDSSF
KRLTYTRYADDFICGVIGSKEDAHRIKADIKDYLEAVLKLELSVEKTLITNARDKAKFLG
YHLYIRQSNLAKRDSAGRLVRNYTGRLVLEVSIETIRDRLLSYGAMKMTYHRGYEVWKPT
ARYFMKDCDDLEILERYNAEIRGFYNYYCIANNSSILHRFKYIMEYSMYKTYATKYRTTK
SHIIRKYKKDGQFSVQYIGRKGDTMTRYLYNGGFKRQKKSFLENDNLPNTAKYFSRTNLI
DRLKASRCEYCQATDSSLEIHHVRKLKDLKGKTFWERLMISRQRKTIALCKDCHKKLHHG
KLD
>orf02208
(SEQ ID NO: 1462)
MEVMKLLAMFRGTIPKDREKMDLFLRYQAQHFDEKWQDLVESFLAEEGKIEEIPHVYSFH
QDIISFLEASSENNDQDLESYTRNFGQAGLSKLSQLSNFEKNLVLEVATYNLSTRFYIQS
EKEKLEPLSELVCLQNQDVNLVNVYRVANNLSDRISRDIEEFLLMVDSKELTKEVLEIHF
EEKEGDVLAYLGSELMATLDTVTDLVHHEENYTQLPLTQKLKIITHFDDVKARSEKSNQV
EEVLSPSSDIEQETEETNSFSNVDKIVEEALREYPIGSQVSYKGQVFQLVSIENAQLNDL
IRLELFNDSNQLFEENPILYLNSLEEIEQVLSLVELEKEDSEIEIDSSSESQEIDLFSYL
EEEKENEKDKETETLIVGIEETDVPVQDFVFPDDLEDFYPKTNREKIETNIAAIELVKRL
EKEGRQANPEEQELLAKYVGWGGLANEFFDELNPKYETERLTLKSLVSKSEYSTMKQSSL
TAYYTDPMIIRQIWQKLLDDGFEGGRILDPSMGTGNFFAAMPRSIRDKSELYGVELDSVT
GAIAKKLHPNTHIEVRGFEEVPYQNNSFDLVLTNVPFGNFRIADKNYDKPYMIHDYFVKH
SLDLVRDGGQVSIISSIGTMDKRTDNVLQEIKTNTHFLGGVRLPDTAFKSIAGTRVTTDI
LFFQKDQAKNLNEEELVFSGSIPFEEDKRVWINPYFDGKYNTQVLGEYEVRNFNGGTLNV
KGVSETLSTDIMKALENVEAPKQIDNFLKAPVFIQEEVDNSLPSRIREDLALYSFGYERN
QIYYRDTHGIRKSSKVDEISYYVDEKGDFKAWDSSLSEHKIDRFVQLHLTDEEALDVYKS
EEASKRGKYKGLFKKTVFYESPLSDKDISRIKGMVDLRETYQSLIEIQRHQDYSRTDFQV
LLSKLNHNYDRFVSQFGYLNASVNRNLFDSDDKYSLLASLEDEYIDSKDQKVKYKKSLAF
EKALVRPERVIARVSTALDALNSSLSDGRGVDLDYMVSIYPEHSQAAILDELGDQILIDP
ESYLRGERKYLSKNQFLSGDILNKIEVVQLLVEENNQEYDWNHALDLLESVRPPRIHLAD
IEFKIGSRWIPQSVYGKFAFECFTNREFELSSPDVEQVIEANPVDGQVHLRTSFAYRYPS
AKDSSLGVSGSRYDTGRKIFENLLNSNQPTITMTVTEGEKKKTITDLEKTSVLRAKEQHL
QELFQDFVSRYPEVQQVIEESYNRLYNRTVSREYDGSHLVIDGLAQNISLRPHQENAIQR
IVEEKRALLAHEVGSGKTLTMLGAGFKLKELGMVHKPLYVVPSSLSAQFGQEIMKFFPTK
KVFVTTKKDFVKARRKQFVSRIITGDYDAIVIGDSQFEKIPVSKERQMNYIEDKLNELRE
IKTHSENKYTVKEAEQSISGLEKQLEELQRFNRDSFIDFENLGIDFLFVDEAHHFKNIRP
ITGLGNVAGITNTTSKKNVDMEMKVRQIQEEHDFKNIVFATGTPVSNSISELYTMMNYIQ
PDILKRYQVDYFDSWVGAFGEIQNSMELAPTGDKYQPKKRFKKFVNLPELMKIYKETADI
QTQDMLDLPVPEAHIIPIESELTENQKLYLEELVMRSDMVKCGTVDPSQDNMLKITGEAR
KLAIDMRLLDSSYSLADNHKLLQVVDNVERIYREGMENKATQMIFSDIGTPKKKDNGFDV
YSEIKALLVDRGIPSKEIAFVHDANSDEKKNSLSRKVNAGEVRILLASTEKGGTGLNVQS
KMKAVHHLDVPWRPSDIQRASVKAV
>orf02209
(SEQ ID NO: 1463)
MEGIYQRDSDQDGLTDAQELALGTNPLSADSDGDGRSDLVEVEEGTNPLEKDLQDIDQTS
ITEPSSVFMEMKQKISDMMESHYKEFIQALISIETGIENEQDLEDLYTYYMRTDSISLLS
SDLETSPQKVEMEIEL
>orf02210
(SEQ ID NO: 1464)
VADTRTKSDSNLGRKGQAVADTRINRLTRWLTDSVNHLFCEENMANSRDYRNPNYTEKIK
LQRFFTQLQIAASFFKEHFVGKIMYYETEIESVELHFSPTNFMHLCGVDYRKGAGSFFDD
CLNRHVIIDELKIKKDGTTMQKLQVLGSIEELLGKHVHLTGSGRYLYLEFDYALRTRKQI
LALTLKETSRKIVPQSLLDLKRKTVFPKGQKVISIYSKHLQTSELFYYLKD
>orf02217
(SEQ ID NO: 1465)
MKDKREIIRARKAFRRSLKDEKKFLKKGKKEVKKQKKDSAVLDEKAWKKEIKKKLEEMRE
ASKARVKQANEDYNHILQNSPPSLLNRKELRDRRLPHARKRLKIAKKQYREAKVEAKEER
KESRKERKTNQKFLYGQESKAKSNFFFQGKSLEELKVKKEVKTAKENLKSTKQAYKSKKV
SRKAKTFLYVLGREGGELASENEDLDGYRTLQETIRKGKRYSRLSYNLGKASVKTGQATC
RFTKKRLTNTKERYHHFKDGKGWKLAKDKPSSFKNRFRKLKKQGLTSVRNIYQKLKAAFS
FFTFAAGNPVTWIVGGIVFLLLLIMSFFLGFSSASLIQQDEFELTKAYTHLTWEDAEHTR
TNDKGITYYTKVDDVMGYMNFKFHDYELHKPVHLFSSETYKDYLSTLWHDLNDGEDLKSM
QDLYETPKYKLSKDDQEEMKELKEEGIYASMQELDNPFEGKSNEDSLTMTYRYGYYDLDG
KPTLQEYILLEAKAHQTIVAPMDGVVSLDGDNVILTNGKGENESRLTLYSIHNGRAIEGT
RVLTGDIIGETPDDTGLKVSYQKYKNKKEKLVYVNPQFYFPKVIQLQTTILPAIGQFGGD
EFERAKHIYEFLKSQGASPQAIAAILGNWSVESSINPKRAEGDYLSPPVGATDSSWDNET
WLAIGGPAIYSGAYPNILHRGLGLGQWTDTADGSTRHTALLNYAHSKNKKWYDLDLQLDF
MLHGDSPYYQSWLKDFFGNTGSAANLAQLFLTYWEGNSGDKLLERQTRATEWYYQIEKGF
SQTNGGQAKSDPQSLEGVRGDLYDHSVPGGGDGMAYAYGQCTWGVAARMNQLGLKLKGRN
GEKISIINTMGNGQDWVATASSLGGETGSTPKAGAIVSFVGGTHGTPAIYGHVAFVEKVY
DDGSFLVSETNYGGNPNYTFRKISQADSAISFAYTTK
>orf02219
(SEQ ID NO: 1466)
MTYKKEEVKGKKEEVLPSTANTISYQALYQNGLMQVKEDYFSQSYLLGDVNYQTVGLEDK
GAIIEKYSDLINSLDDQTNFQLTIFNKRLNLEKFRQSVLYEEKEDGYDTYRKELNRMMNQ
NLDSGENNFSAVKLISFGRKDSNPKQAYRSLSQIGEYFKSGFSEIDARFESLAGEERVNL
LADMLRGEHHLPFSYCDLTRSGQTTRHFIAPNLLDFKNKNYLQINDRLLQIVYVRDYGME
LGDQFIRDLMQGDLELIVSLHAQSSTKADAMKKLRTKKTLMESQKIGEQQKLARTGIYLE
KVGHVLESNIDEAEELLKTMTETGDKLFQTVFLIGVFGQDEEELKQALDTIQQVAGSNDL
MIDKLPYMQEAAFNCLLPFGCDFLEGVSRSLLTSNIAVNSPWTSVDLQDRSGKYYGINQI
SSNIITIDRSLLNTPSGLILGTSGAGKGMATKHEIITTKIKESGENTEIIIVDPEAEYSV
IGRAFGGEMIDIAPDSQTYLNVLDLSEENMDEDPVKVKSEFLLSFIGKLLDRKMDGREKS
IIDRVTRLTYQSFKEPSLEEWVFVLSQQPEEEAQNLALDMELYVEGSLDIFSHKTNIQTG
SNFLIYNVKKLGDELKQIALMVVFDQIWNRVVRNQKLGKKTWIYFDEMQLLLLDKYASDF
FFKLWSRVRKYGASPTGITQNVETLLLDPNGRRIIANSEFMILLKQAKNDREELVQLLGL
SKELEKYLVNPEKGAGLIKAGSVVVPFKNKIPKGTQLFDIMSTDPDKMASN
>orf02220
(SEQ ID NO: 1467)
MNTRVFKDISKYQHRAWLGFTTRQIIFVLPAFIVTIIVLGLNLFFWQFGDWFVYGFVFAF
TIPLMLFGVYKPNDLYFEHYLKYRLHFELTVPLRTITGKKGHEHEKKIKYIKETKSFNDL
>orf02221
(SEQ ID NO: 1468)
MNLSLVSPFVYLASEKISAENLFEGFSVDLQSTVDLIKSLSSYNPTVWTYMSSITKSVMQ
PLGVAILSVVLILEFSKMAKKIANSGGAMTFEALAPMLISYIMVAVVITNTTVIVEAIIG
IASHAIEQVASIVAHGGAKYDTISGLKGSGFIGRMIVGFFALLIWLVRIVSAAMVNLLVS
IRFIQLYLMIPFAPLTIPTFLSDEWKSIGIGYLKNIMVYAVQGVLIFLIVSLVPLFESAG
KIAVSNGAGVLQSLAIMFGSLVQAILLIIALVGSQRTARSILGM
>orf02223
(SEQ ID NO: 1469)
MITHFKGFVYGVDASAMFAQAMSLLQKGLIAVGAFLVVVGIVNLATNIKDGGPGVRNAIL
EIVGGVMVGAAGAFVTQISI
>orf02224
(SEQ ID NO: 1470)
MMYSGKKFLLFSLLGILLGYLFHRLTLLYDSYTGNSLDKWTHLLMEGQDEVLQSPWNVSF
TGKSSAFFLLGFVMMLLVYLYLETGKKQYREGIEYGSAHFGTLKEKKLFYGKEFSHDTIL
AQDVRLTLLDKKPPQYDRNKNIAVIGGSGSGKTFRFVKPNLIQMNSSNIVVDPKDHLAEK
TGKLFLEHGYQVKVLDLVNMKNSDGFNPFRYIETENDLNRMLTVYFNNTKGSGSRSDPFW
DEASMTLVRALASYLVDFYNPPKTREQLIEESRLSQKEHQNLLKRQKKEVEERKKRGRYP
SFAEISKLIKHLSNGENQEKSVLEILFENYAKKYGTENFTMRNWADFQNYKDKTLDSVIA
VTTAKFALFNIQSVMDLTKRDTLDMKTWGKEKSMVYLVIPDNDSTFRFLSALFFSTVFQT
LTRQADIDFKGQLPLHVRVYLDEFANIGEIPDFAEQTSTVRSRNMSLVPILQNIAQLQGL
YKEKEAWKTILGNCDSLVYLGGNDEDTFKFMSGLLGKQTIDVRNTSRSFGQTGSGSLSHQ
KIARDLMTPDEVGNMKRHECLVRIANMPVFKSKKYNSTKHPNWKYLANQETDERWWDYQI
NPLNQSQENHLEGLRIRDLTFESSLK
>orf02225
(SEQ ID NO: 1471)
MSSEQQERMAVQYAERSLLFTVKSLLKILEWSRRQALAQDSAYKIGVQKLEELLQSPYSI
DTINLKKDFLDKPIDIEKFKAFLEKEEIPLAIAWQGDSLHFYTKDRSILDNHLDQLLEKM
VNDPEKLADFTMDKSLDDAIDEAKSQITFRQEGAVKQKEMVR
>orf02226
(SEQ ID NO: 1472)
MKVVNLYDLKQMGNKGGCTIQLIHHFPFGMGLGHLKKDYIEFKRVGIVDGKAVEVTLREP
YSRDLLQVVKSIKQRQKLIAYRYKEGKLLFVKEEASDVL
>orf02227
(SEQ ID NO: 1473)
MFSNANSFKAKIKNISKDKGIPAQQVQQHYLIEQVLKLISTSSYRDSFIVKGGYLIGQMI
GLDKRTTMDLDVTLKGTEMSRENLIHIFEEILCSKTDGFSFSVDKLEPIRQDDEYGGFSL
KLNATFDTLKEVVFIDITTGDKITPREITYSMTSIFTNESIKIWTYNLETVLAEKLETII
SRGLASTRPRDRYDLFTLYKLRKEEINLEVLKNALENTAEKRKSKDTIYNWEEQVRGIEI
SDYQKELWIRYQRQFKYAKDISFDNSVQVIREIMQQIF
>orf02228
(SEQ ID NO: 1474)
MVDKREKLMNSFNQYGFLTFKQVIDENLHYKTLLKMVAEGKIDAEEKGLYRLPDIYLDEW
FVLQYRFPKGIFSLETALWLHGLSLTIPFNMTMSFPYGTNTKNIKEADICPIILRSHYSE
GIIEIERLPGQFIKVYEVERVLVECLRPVHQVDLQIIAPAFKKYFQQNKIHLHKLFYYAQ
LFKVTDKLQSYTEVLS
>orf02229
(SEQ ID NO: 1475)
MRCLFFYPILKGSELMKTKNQESKGRSPLFKTIKHSFSQ
>orf02230
(SEQ ID NO: 1476)
MELKFVIPNMEKTFGNLEFAGEDKVVQRRINGRLTVLSRSYNLYSDVQRADDIVVVLPAE
AGEKHFGFEERVKLVNPRITAEGYKIGTRGFTNYLLHADDMIKE
>orf02231
(SEQ ID NO: 1477)
MRLANGIVLDKDTTFGELKFSALRREVRIQNEDGSVSDEIKERTYDLKSKGQGRMIQVSI
PASVPLKEFDYNARVELINPIADTVATATYQGADVDWYIKADDIVLTKDSSSFKAQPQAK
KEPTQDK
>orf02233
(SEQ ID NO: 1478)
MKQRGKRIRPSGKDLVFHFTIASLLPVFLLVVGLFHVKTIQQINWQDFNLSQADKIDIPY
LIISFSVAILICLLVAFVFKRVRYDTVKQLYHRQKLAKMILENKWYESEQVKTEGFFKDS
AGRTKEKITYFPKMYYRLKNGLIQIRVEITLGKYQDQLLHLEKKLESGLYCELTDKELKD
SYVEYTLLYDTIASRISIDEVEAKDGKLRLMKNVWWEYDKLPHMLIAGGTGGGKTYFILT
LIEALLHTDSKLYILDPKNADLADLGSVMANVYYRKEDLLSCIETFYEEMMKRSEEMKQM
KNYKTGKNYAYLGLPAHFLIFDEYVAFMEMLGTKENTAVMNKLKQIVMLGRQAGFFLILA
CQRPDAKYLGDGIRDQFNFRVALGRMSEMGYGMMFGSDVQKDFFLKRIKGRGYVDVGTSV
ISEFYTPLVPKGYDFLEEIKKLSNSRQSTQATCEAEVAGVD
>orf02234
(SEQ ID NO: 1479)
LAYGLSQNRLAVATGITRQYLSDIETGKVKPSEDLQQSLWEALERFNPDAPLEMLFDYVR
IRFPTTDVQQVVENILQLKLSYFLHEDYGFYSYSEHYALGDIFVLCSHELDKGVLVELKG
RGCRQFESYLLAQQRSWYEFFMDVLVAGGVMKRLDLAINDKTGILNIPVLTEKCQQEECI
SVFRSFKSYRSGELVRKEEKECMGNTLYIGSLQSEVYFCIYEKDYEQYKKNDIPIEDAEV
KNRFEIRLKNERAYYAVRDLLVYDNPEHTAFKIINRYIRFVDKDDSKPRSDWKLNEEWAW
FIGNNRERLKLTTKPEPYSFQRTLNWLSHQVAPTLKVAIKLDEINQTQVVKDILDHAKLT
DRHKQILKQQSVKEQDVITTKK
>orf02235
(SEQ ID NO: 1480)
MNFGQNLYNWFLSNAQSLVLLAIVVIGLYLGFKREFSKLIGFLIIAIIAVGLVFNAAGVK
DILLELFNRIIGA
>orf02236
(SEQ ID NO: 1481)
MNGVFLIFIIQADFLFDFLKVNGKGKPRTDQFALIVFA
>orf02237
(SEQ ID NO: 1482)
MYDVARYYIEETGALGEVPASLQNYIDYQAYGRDLDLSGTFISTNHGIFEIVY
>orf02239
(SEQ ID NO: 1483)
MYLIGYAIKFTPNCCNGFLWLVSAKRYFFSCIGQLWSQCISNDRLQKSIRLFTIKSFCRH
KPCESHRNPKVFEKCSLYHGKRGQVAKYHHCKE
>orf02242
(SEQ ID NO: 1484)
MAYPIKYIENNLVWNKDGECYAYYELVPYNYSFLSPEQKIQVHDSFRQLIAQNRDGKIHA
LQISTESSIRSAQERSKNEVTGKLKAVAYDKIDQQTDALISMIGENQVNYRFFIGFKLLL
NDQEFSMKSLTVEAKNALSDFVYDVNHKLMGDFVSMSNDEILRFQKMEKLLENKISRRFK
IRRLDKDDFGYLIEHLYGQTGTAYEEYEYHLSKKKLDNETLIKYYDLIKPTRCLVEEKQR
YLKIQQEDETVYVAYFTINSIVGELDFPSSEIFYYQQQQFTFPIDTSMNVEIVANRKALS
TVRNKKKELKDLDNHAWQSDNETSSNVAEALESVNELETNLDQSKESMYKLSYVVRVSAN
DLDELKRRCNEVKDFYDDLSVKLVRPFGDMLGLHEEFLPASKRYMNDYIQYVTSDFLAGL
GFGATQMLGENEGIYVGYSLDTGRNVYLKPALASQGVKGSVTNALASAFVGSLGGGKSFA
NNLIVYYAVLYGAQAVIVDPKAERGRWKETLPEISHEINIVTLTSDEKNKGLLDPYVIMK
NPKDSESLAIDILTFLTGISSRDGERFPILRKAIRAVTNSEVRGLMKVIEELRVENTPLS
TSIADHIESFTDYDFAHLLFSNGYVEQSISLEKQLNIIQVADLVLPDKETSFEEYTTMEL
LSVAMLIVISTFALDFIHTDRSIFKIVDLDEAWSFLQVAQGKTLSMKLVRAGRAMNAGVY
FVTQNTDDLLDEKLKNNLGLKFAFRSTDLNEIKKTLAFFGVDPEDENNQKRLRDLENGQC
LISDLYGRVGVIQFHPVFEELLHAFDTRPPVRKEV
>orf02244
(SEQ ID NO: 1485)
VKPSIVNRIKSNWTLKRLGKVAMTVAFTLVIAIFLLAMLGTVVQAAGLVDDTVNVANEYS
RYPLENYQLDFYVDNSWGWLPWNWSDGIGKQVMYGLYAITNFIWTISLYVSNATGYLVQE
AYSLDFISATADSIGKNMQTLAGVSANGFSTEGFYVGFLLLLILVLGVYVAYTGLIKRET
TKAIHAIMNFVLVFILSASFIAYAPDYIKKINDFSSDISNASLSLGTKIVMPHSDSQGKD
SVDLIRDSLFSIQVQQPWLLLQYNSSDIESIGIDRVESLLSTSPDSNNGEDREKIVAEEI
EDRSNTNLTITKTINRLGTVFFLFVFNIGISIFVFLLTGIMIFSQVLFIIYAMFLPVSFI
LSMIPSFDGMSKRAITKLFNTILTRAGITLIITTAFSISTMLYTLSAGYPFFLIAFLQIV
TFAGIYFKLGDLMSMFSLQSNDSQSVGSRVMRKPRMLMHAHMHRLQRKLGRSMTTLGAGS
AIVTGKKGQSGSGSSARTQADHSRPDGKEKSTLGKRIGQTIGTVADTKDRMVDTASGLKE
QVKDLPTNARYAVYQGKSKVKENVRDLTSSISQTKADRASGRKEQQEQRRKTIAKRRSEM
KQVKQKKQPASSVHERPTTRQEQYHDEQTSKQSNIQTSYKESQQAKQERPAVKSDFSSPK
VERQGNTVQEKTVQKPATSTTTADRTSQRPITKERPSTVQRVPLQNTRTTNQNRHH
>orf02246
(SEQ ID NO: 1486)
MKLKTLVIGGSGLFLMVFSLLLFVAILFSDEQDSGISNIHYGGVNVSAEVLAHKPMVEKY
AKEYGVEEYVNILLAIIQVESGGTAEDVMQSSESLGLPPNSLSTEESIKQGVKYFSELLA
SSERLSVDLESVIQSYNYGGGFLGYVANRGNKYTFELAQSFSKEYSGGEKVSYPNPIAIP
INGGWRYNYGNMFYVQLVTQYLVTTEFDDDTVQAIMDEALKYEGWRYVYGGASPTTSFDC
SGLTQWTYGKAGINLPRTAQQQYDVTQHIPLSEAQAGDLVFFHSTYNAGSYITHVGIYLG
NNRMFHAGDPIGYADLTSPYWQQHLVGAGRIKQ
>orf02247
(SEQ ID NO: 1487)
MMKFRKNQNKEKQIPKEKKPRVYKVNPHKKVVIALWVLLGLSFSFAIFKHFTAIDTHTIH
ETTIIEKEYVDTHHVENFVENFAKVYYSWEQSDKSIDNRMESLKGYLTDELQALNVDTVR
KDIPVSSSVRGFQIWTVEPTGDNEFNVTYSVDQLITEGENTKTVHSAYIVSVYVDGSGNM
VLVKNPTITNIPKKSSYKPKAIESEGTVDSITTNEINEFLTTFFKLYPTATASELSYYVN
DGILKPIGKEYIFQELVNPIHNRKDNQVTVSLTVEYIDQQTKATQVSQFDLVLEKNGSNW
KIIE
>orf02249
(SEQ ID NO: 1488)
MKIINIGVLAHVDAGKTTLTESLLYNSGAITELGSVDKGTTRTDNTLLERQRGITIQTGI
TSFQWENTKVNIIDTPGHMDFLAEVYRSLSVLDGAILLISAKDGVQAQTRILFHALRKMG
IPTIFFINKIDQNGIDLSTVYQDIKEKLSAEIVIKQKVELYPNMCVTNFTESEQWDTVIE
GNDDLLEKYMSGKSLEALELEQEESIRFQNCSLFPLYHGSAKSNIGIDNLIEVITNKFYS
STHRGQSELCGKVFKIEYSEKRQRLAYIRLYSGVLHLRDSVRISEKEKIKITEMYTSING
ELCKIDKAYSGEIVILQNEFLKLNSVLGDTKLLPQRERIENPLPLLQTTVEPSKPQQREM
LLDALLEISDSDPLLRYYVDSATHEIILSFLGKVQMEVTCALLQEKYHVEIEIKEPTVIY
MERPLKKAEYTIHIEVPPNPFWASIGLSVAQLPLGSGVQYESSVSLGYLNQSFQNAVMEG
IRYGCEQGLYGWNVTDCKICFKYGLYYSPVSTPADFRMLAPIVLEQVLKKAGTELLEPYL
SFKIYAPQEYLSRAYNDAPKYCANIVDTQLKNNEVILSGEIPARCIQEYRSDLTFFTNGR
SVCLTELKGYHVTTGEPVCQPRRPNSRIDKVRYMFNKIT
>orf02250
(SEQ ID NO: 1489)
MKPSSFQTTIENQFDYICKRAMEDERKNYMLYLSRIAKREVSFSDVGDYLVSQFATTDNY
STDFQIFTLNGLSVGVENDLLSEALRELPDKKREILLLFYFMDMSDSEIADLLKLNRSTV
YRHRTSGLALIKKFMEEFEE
>orf02251
(SEQ ID NO: 1490)
LATLDCVQCIYNFFKLFSFNLNTIAIHNQPICIFIFLCQAS
>orf02252
(SEQ ID NO: 1491)
MSEKRRDNKGRILKTGESQRKDGRYLYKYIDSFGEPQFVYSWKLVATDRVPAGKRDCISL
REKIAELQKDIHDGIDVVGKKMTLCQLYAKQNAQRPKVRKNTETGRKYLMDILKKDKLGV
RSIDSIKPSDAKEWAIRMSENGYAYQTINNYKRSLKASFYIAIQDDCVRKNPFDFQLKAV
LDDDTVPKTVLTEEQEEKLLAFAKADKTYSKNYDEILILLKTGLRISEFGGLTLPDLDFE
NRLVNIDHQLLRDTEIGYYIETPKTKSGERQVPMVEEAYQAFKRVLANRKNDKRVEIDGY
SDFLFLNRKNYPKVQVITTA
>orf02253
(SEQ ID NO: 1492)
MGHANIAMTLNYYAHATFDSAMAEMKRLNKEKQQERLVA
>orf02254
(SEQ ID NO: 1493)
MKRIIPVYIFQQVNVLLVSLYLLKLLCISELTILQILYCASLISFLWMYGQRKQVVKVNM
KTRMKWLGIGFVSLLIINLCFSLIHAQGTTNQANLIGLQHQVPWFSFLLLLINASMVEEF
LYREILWNLVRKLDIRVALTSILFVLAHHPGTILAWCLYVSLGMFLGLVRYKSDLWGSMG
LHLVWNLSVYVLFFL
>orf02259
(SEQ ID NO: 1494)
MKRITANQYQTSERYYKLPKILFEDEKYMDMKLEVKVAYSILKDRLELSLSRGWIDEEGA
VYLVFSNSKLMKLLGCSKSKLLSIKKILKEYDLIDEVQQSSSEKGRLANKIYLGELSSTP
VASSNRPSVKKKIGQVENETAPVSHSAPSETEVSETKYSETDSLFSEDEEERYTQPILKR
KVEKVTKYDQDYIWGLVQDQFRREGFSETASEIAMTDFERIYQYALDNVRFVRRAEVLAE
FVFNGLYSVWNNRVRKGGG
>orf02260
(SEQ ID NO: 1495)
MTKELQSSRYIVISFLVREMGIDIVEAISLMAELEKSGLVRLESSGDLILKELGGAL
>orf02261
(SEQ ID NO: 1496)
MIVILLSFFLQKIKKGEQYSTVLQNIFIKKKNPAKLIFGRVFGRKLN
>orf02276
(SEQ ID NO: 1497)
LQVWYNLQSDFEQEITLIMWNPFANLVFNQPLISFFADLNLKILGYSYTDRVKTWPDIGT
GCRYNNLHLILLAP
>orf02314
(SEQ ID NO: 1498)
MVICHNDYLLWLPEFSQPLTSLSQTTFFNLNIIRMMRNIDSDFHRRISFSLLVFFC
>orf02318
(SEQ ID NO: 1499)
LIEGHLVFADKPAQALVLLRKVGSPKKVSFLTLHLYFLILKIDILKITGF
>orf02324
(SEQ ID NO: 1500)
MFLHHLLQIKGGLGIQTSQGFVQNPNIRSRQECPNDKDFLTHSVRKSFNNLIAVFSKIKN
VQ
>orf02326
(SEQ ID NO: 1501)
MTNQAHNLSIFNLQIEITQGLFITIQLTNILKFNHGTTPIFICIIHRYTSTLIQKNQYFI
LSS
>orf02348
(SEQ ID NO: 1502)
MAVTKSQVFSRQGFDFSILGQDLTRLQDVSNLATIGTRIHKDSTANASWNTTSKLKAS
>orf02349
(SEQ ID NO: 1503)
MTEGNASCFNQVSPSFCFNGIAINRNVIELVTQDDKSTNPTITNDDIACIAKNHPRDIFL
VGKFHNASQLKTISWKDQIISLSTYFCITIAMQGFLKTDINSF
>orf02361
(SEQ ID NO: 1504)
MRLRDLRRVDFPDPDGPIKAVISLGWKDRETLFKAFFLL
>orf02364
(SEQ ID NO: 1505)
LKNHSNVFTHFINVDFWTVDINSTIENLPSYFSNINGIIHAIETA
>orf02365
(SEQ ID NO: 1506)
LHINPLNGFIFTIVNMDILSRKGYFFFRKGKDMLLIPVIC
>orf02387
(SEQ ID NO: 1507)
MTIHIQVVKTNMVILADRFFQGLILRSTDKFFIKIRLVRSHNLRFNNMDFSTVAVHENKG
RHHVDELLLRFIINSKATVAKKSIVAQGFRFDGNFFRKTRQTNHLNIIFYDNPDQIIFFQ
NGLITNSQFNRLHP
>orf02404
(SEQ ID NO: 1508)
MVKRRIRRGTREPEKVVVPEQSSIPSYPVSVTSNQGTDVAVEPAKAVAPTTGWKQENGMW
YFYNTDGSMATGWVQVNGSWYYLNSNGSMKVNQWFQVGGKWYYVNTSGELAVNTSIDGYR
VNDNGEWVR
>orf02420
(SEQ ID NO: 1509)
MRFIVGRFTSFSLGIEFSPTSKLDDLLFKIAFLMILATWIKARKTKEAT
>orf02424
(SEQ ID NO: 1510)
MANDNKSHYLIYRVLGISFEEGENIDLYQNKGRFLYKYAGSFLEEAAVLSFNEKFGTENT
>orf02433
(SEQ ID NO: 1511)
LHYRTTPTLIMVVQRDCLILSFPRQKGPIVGQMAIIL
>orf02435
(SEQ ID NO: 1512)
VFALLDNSTFLRKSLHLRKMEMFPVETEEITYKRKKSKGKRQAILAQFDSEEVHHQVEES
ICPDCQDDLKEIGASLQRQELVFIPAQLKRVDHIQHAYKCQTCSKNNPSDKIVKAPIPKA
PL
>orf02451
(SEQ ID NO: 1513)
LVCQTIKYWHKFHLHIGRCKLLIGLIPVLNFFIRADIDCLLVLLSLIDRQNGKQFNLCQW
IIASNGLNDSFEIIESLIHRNILSDIICPNQKKNFIYCSTI
>orf02459
(SEQ ID NO: 1514)
VGHNSGSFFLFLLLRLLLSPLLRNSISFLTSQGIPWKLSNNKTKPIDKPTASKSIATNPL
LLHLR
>orf02480
(SEQ ID NO: 1515)
LDFLNHLWVAHAGNSSSCTNISRNSFQGHHSCRTSSFCDTSLFRILHIHNNTTLEHLCQV
FI
>orf02505
(SEQ ID NO: 1516)
MLLLISLTQLIIFLFFERFNLLLKTFLLVDLKSNKSA
>orf02517
(SEQ ID NO: 1517)
MGFSMKLIHDLNTHTTHSTAKMLHNVKAIKNDFSIRE
>orf02524
(SEQ ID NO: 1518)
MQEHYTPKGKHLTIDNRRLIERWKNENKSNREIAGLLGKAPQTIHTEVKRGTTLQQVRKG
LYKKVYSADYAQTVYQFNRKRSVKKLILTKEIREKILHYHKQKFSPEMMVNKKQVKVGIS
TIYYWFHNGHLGLTKADMLYPRKRKGVKKQASPNFKPAGKSIEERPDVINLRLENGHYEI
DTVLLTKIKNYCLLVLTDRRSRHQIIRLIPNKTAESVNQALTLLLGEHRILSITADNGSE
FKRLSEVFPEEHIYYAHAYSSWERGSNENHNRLIRRWLPKGTKKTTPKEVAFIENWINNY
PKKCLDYKSPSEFLLGG
>orf02527
(SEQ ID NO: 1519)
VVPRYVTKHQGWDHNSHTITNSDDDPATLVTFRTFKFNVGNCTIPKNDQNGSSQKFSGIL
>orf02534
(SEQ ID NO: 1520)
MAYSTDFKQRALDYIKEGHSHVEAAKFFGVGVRTLFTWEKKDVNKDT
>orf02585
(SEQ ID NO: 1521)
MAVQANWSFDITHDSSFFFSNQKRGLNFSQMCFKDRRRNGFFDRKIFKFKFNNPIQIF
>orf02595
(SEQ ID NO: 1522)
MEIVLVSFSISFQHFIIAYCLDFSSAGFRNSQNFSNFC
>orf02608
(SEQ ID NO: 1523)
MKLKLLRVDTKVIMGSFLLVLSSLLALLLPLILKGLIDGSSIENIGSKVFQSFLIFIGQA
LFSSIGYYLFSQSGEKKIAKIRKKVIEGLIYAEKSFFDKSQSGELTSAIVNDMSVIREFL
ITTFPNIILSLVMVLGSIVVLFSLDWNLSLLLFITLPCMMFIILPLSNISEKYSRRLQEE
IGFLTGQLTEKIQEHELIKTNQAEKSVQDVLDNCIERVQNNSLKSDRVTSFETPFALLFI
FATIVVMLTYGGYRVSAGYISVGTLVSFLIYLFQLLNPISNIANFVTIYSSSKGSSVALE
NLLAVPKEKFEGGKSVSGQGLNFNHVYFGYDENRPVLKDITCSIFKGQKIAFVGPSGSGK
STIVRLLERFYKPLSGDILMEQSSIYDFNLKEWRSKIAWVSQNNAVLSGSIRDNLCLGLN
RLVTDDELMKVLDLVSLGDEIRSMKEGLDTEVGERGRLLSGGRSQRLQIARAYLKDAEIL
IFDEATANLDADSEYAIISSLYSVLKEKTVVIIAHRLSTVKDVDCIFFLEEGKITGSGTH
KELLENHERYARFVQEQMIE
>orf02621
(SEQ ID NO: 1524)
LIRYLDQYEDVILREIKAQFPDVAVDKLMEEYIKAGLILRENKRYYLNFPTLESLDSLEL
DQEIFVREASPVYQALLEQSFETELRNQINAAILVEKTDFARIKMTLSNYFYKVKQQYPL
TEKQQELYDILGDVNPEYALKYMTAFLLKFLKKDQLMQKCRDIFVDSLVVLGYIVQNEDG
KYELAIDFDKERLTFYLA
>orf02622
(SEQ ID NO: 1525)
MIGLKEVCRFLTDNTSLSTSMINHPIQINGNMAIVTCGSLDGLSHV
>orf02633
(SEQ ID NO: 1526)
MRERVRLSGSLFTSLKTREHIKSTMELFHKYVFFLIQEIKIKMINFLKIGDLPTL
>orf02643
(SEQ ID NO: 1527)
MIDHFEIKVKDLQISEGFYRSFLAPLDYKLAFKTSSLISFLSPNSPHPGGDFWLTQGTQD
PVHFAFLAENKEEVQACYEAGLEAGGRDNGAPGYRSEHPIYYAAFMIDLDGNNIEVVCHK
E
>orf02645
(SEQ ID NO: 1528)
VIVFLSRNKDGNAFCHLDLISIANPVWGWDDDFITWIDHSHKEGIERIFGSRSDCHLI
>orf02648
(SEQ ID NO: 1529)
LSNQFYFSLQTKPILKVKQFLLFQSQMIRVSEILQFSNKL
>orf02652
(SEQ ID NO: 1530)
MKHSHKKSFDWYSMQQRYSIRKYYFGAVSVLLGTALVLGAAASVQTVQAEENKQETTNSI
SVGRGEAATKPAEVSASNKEKTYAAPTVANPVETTPVKTGEVTKPAEKVEEAKDKKEEVT
HQDAIDKSKLLTALSRAKKLESKLYTEASAANLQTSIQAGQSLLGKADASEAELSAAESS
IQSSIIGLELRSNSNKGTVSETPVAKKANIVEAKEETKPAVTTTERSAVDSAILPISTAA
KVETTSAPASTNEILKPSLSLSDARQNPAIRKEDVDRGYSGFRTAGSGFRAAGSGFRAAG
PENKPILNPNNTIAFSDISQGLHSFRGIGHSRGGREIHYDVTTVRRGNRLNFTIKYSGPG
EFVNNNFILDKGDGFGNPSNATITSSNPRVREQSKSISQGANYVSHSGYSMTSATSTNTE
QTIRFSLPIINPNGDLSVRLKPVTFNVDQGGGGAATSNDPYSNSNYYHRANPLLLDANPY
GGTNNKTVSEDIDFQTVYLPTSKLPEGQTRLVREGEKGQRQITYKVHRFGNETLLGLPIS
NRVTKEAKPRIMQIGVAKELIDTVKPRVDQNKVGDTNNLTFYLDNDGNGVYTEGVDELVQ
KIAIKDGAKGEKGDQGERGLTGAQGTKGEKGDQGERGLTGAQGAKGEKGEQGFQGRDGEQ
GPKGEDGKTPTVKVTDGQDGTHTITINDGKGGITTTVVRDGFDGASPLVSTHRNEADKTT
TVIFYYDLNDNNQFDEGDTKLKEVVIADGKQGPKGDKGDNGKDGFTPEVTVTDNNNGTHT
ITITQPDNRPSLTTIVKNGEDGKTPKVKAERDDAKKQTTLTFYIDKDGDGSYTAGKDELV
QTTVVKDGQDGAAGASGRDGKEVLNGKADPTTEGKDGDTFVNTQTGDVFVKKGNTWEPAG
NIKGPKGDKGADGAKGEKGDRGERGLTGAQGTKGEKGDQGERGLTGAQGTKGEKGDRGER
GLTGAQGQAGRDAVTPTVTVKDNKNDGTHTITINDGRGNVASTVVRDGFDGASPLVATQR
NEADKTTTVIFYYDQNGNNELDASDKKLKEVIIADGAKGEQGLQGRDGQDGAQGQAGRDG
KDVLNGKANPEVNQGKDGDKYVNTETGDVFVKNNGNWDKEGNIKGPKGDKGERGEDGKTP
EVTVTPGKDGHSTDITFTVPGKDPVTFTVKDGKNGKDGRAPKIKVEDITSPSRIRRDTDA
AATPTRNGIRVTVYDDVNDNGVYDEGVDKVLNSKDIYNGIDGRDGSAPTITTKDNGDGTH
TITVQNPDSSESTTVVKDGKDGKTANITTTENPDGSHTITVTNPDGSTKETVVKNGKDGK
TPKVEVTDNNDGTHTVKVTDGDGNVTNAIIKDGKDGKAATATTTENPDGSHTVTITNPDG
TKNEFVVKNGRDGVDGRTPTASVRDNGDGSHTIVITNPEGVTTETTVRDGKSPKVTITDE
QNGTHKISVLNGDGTTTETIIKDGKSPVATVRDNQDGTYTIRVENGNGTVSETTVRDGKS
PTAKVVDNGDGTHTITVVNSDGTTTTTTVRDGREPKLEVIDNNDGSHTIKVTGADGKGTT
TTIFDGKSPKANIVDNGDGTHTLTIVDSDGREYKSIIKDGKDGKDSVSPTVTVKNNNDGT
HVVTITNPDGSKTEMVIKDGKDGKCGCQDKPVTPSNDKPVPPTPNVPTPEVPVKPVPAQP
TPNVPTPEVPVQPTPAVPTPEVPVKPVPAVPEQPVVPTPAQPATPVNANPVAPTTGKENR
GDKLPETGSQSDYISVLLGSGILLSLYVGRRKED
>orf02654
(SEQ ID NO: 1531)
MRNLLSTKVQRQLRLMETLIQNRNWMKLHELAEKLGCTERILKSDLNELRIAFPSINIQS
SVNGIMIDLEVNTSVEDIYQYFLANSQSFQLLEYMFFNEGLPIYRTIENLYFSSANLYRL
GRNITKVLSSQFQIELSFTPSEIRGNEIDIRYFFAQYFSERYYFLDWPFPDLPEEDLTEF
ADFFYKITNYPMRFSIYRMYKLMIAISIHRVKNGHFIDLPNHFYKEYYPLLKSIPNFQET
LAYFSKHFGLEMTPDTIAQIFISFLQNDIFLDPQEFFNSLEDNSQARYSYQLLSQILERL
SKQYKITFTNHDELIWHLHNTAFFERQEIFSTPILFEQKALTIKKFEVYFPDFMGSARQE
LAQYRQAIGQHDHPEQLEHLMYTILTHAENLSTQLLENRPPIKVLIISNFDHAISLTFVD
MLSYYCNNRFTFDIWDELKTSPEILNQTDYDIIVSNFYISGITKKFICRNHLSIMNLVNH
LNTLSNEIHLSNTL
>orf02655
(SEQ ID NO: 1532)
MIFKIGLFYLGQFVSLDMTVHKPIKKLQGWVVLSSLPFQSLDILTFFRSLLS
>orf02657
(SEQ ID NO: 1533)
MGFYLMVASMLLGLLALKIGFSQFKENKDKFLSILTSLAGLALVLVAVWLGWPK
>orf02677
(SEQ ID NO: 1534)
MLDSDIGCSRKNLLGLFWIRRRRNIHIVDRAMEKGISNRAPNKISLKACFFNFF
>orf02696
(SEQ ID NO: 1535)
MAFNQFNRCITLSIPTAPNIPTSVVHRTYLHDATVPNNVREKT
>orf02698
(SEQ ID NO: 1536)
MQQITEIIIAFATSFLTVAVGGIVKAVKDYLLRKGGEKAVIIAEILAKNAVHAVEQVASE
TGYKGEEKLEQARAKVRAELTKYNISMTDKDLDTFVESAVKQMNDAWKGR
>orf02699
(SEQ ID NO: 1537)
MKIEFFNFLRSVIQTEDGLVLYALALIVSMEIIDFVTGTIAAIINPDIEYKSKIGINGLL
RKISGVLLLMILIPASVLLPEKTGFAFLYSICLGYIAFTFQSLIENYRKLKGNVTLFQPI
VKVFQRLLEKDDDTKKGE
>orf02700
(SEQ ID NO: 1538)
MLKVTKTRQLVTEFFAQDGDQQKLVKTTVINTDNKAVSTISETLHDPDLYANNRISMRKH
EQELREMRYKIEDAILAELEADSEHKE
>orf02702
(SEQ ID NO: 1539)
MTKFINSSGSLHLNIYIEQVSQDIANNSSRVSWKATVDRDGAYRTYTYGNISNLSVWLNG
SSVHSSHPNFDTSGQEFTLASGEVTIPHSGDGTKTFAVWASFDPNNGVHGNITVSANYTL
SSIPRSSSVSDNALSGNRRLGSPHTLTIDRKSSSFTHQVWYRVFGSNWIDLGKNHATGVS
FVPNIDLARYNTKAKSGTMDICVRTYNGTTQVGNDVYSNGWYFEIPESVKPTFSGITLTD
MNTVARQLLSGNNFLQIISDIQVNFNNPSGAYGSTITGYRAEIVNKNQVTTVNGGRLGMM
NFNGSATIRASVVDSRGRQSDTRDITINVIEYFAPAFSFTAFRTRETPNIIQVVRNAKIA
PITLSGSQKNVMTLSFKVARLGSTTFTADHGRASGIWTTQHTLNNSAANMAGNYVATKSF
VVIGTLSDKFTSTEFTATVATESVVMSYDKDGRVGIGKVAEQGGAGSLDVLGDIYARNKP
IQQYQLTDNNGCGKLIKQDFNTMKETGTWWINGSSQNNPFSGTWGMLEVFRPNPGSHERI
QRFTTSTGYMAVRENGYDNNWRPWRYLVQQSKSTNNSDYVALLKSESTPTPWQNAILQNG
WNHHRDYGGVQFSKTFDGVVCFKGTCKGGKIARESIILTLPEHFRPSTTLFKTALNNDYG
SAVIGIYPNGNVVVKSNVDATWLNFDNVFFKI
>orf02709
(SEQ ID NO: 1540)
MLLTIHDANLQKVAFIDNEKQGTLNYYDDTWTRSLATGSSTFEFTVFKKAVKSDLPLAKA
YHHLNEHAFVSFKYKGKSFVFNIIIVEENEQTIKCYCENLNLELINELANPYKSNKAMTF
KEYCEAMDLLNYTHLSIGINEISDYKRTLEWEGQETKLARLLSLAKRFDAEIEFDTQLNA
DSTIKKFSVNVYHENDDNHQGVGRVRNDVIVKYGKNIHSITRKVDKTGIFNTIRPTGKMP
TVEEELSGDKGSKSETVKNADGSTTKTTISTASDGTKSKTIVHTKVTKLADKTRITTTTT
TRSDGSIEQTVTTSKKGGASTSETKVLKKPNPKEKTNTTEDVLTIEGLDEWEVKNEKGIV
EFYQRGQALYAPISMQLYPSTFTHSTGELDQWTRKDFHFETDEPNELRRLGYLKLKKYCY
PAITYEVDGFVDADIGDTVKVHDDGFAPLLMIQARVTDQKISFTNPVRNKTIFDNFKALE
NKLSADIQSAFERLFEAAKPYTIKLSTDNGVIFKNQIGQSLVTPTLYKGGKPVVVGVTWR
WALDGEVTTGMTYLVRGSNVTDTVTLTVAAYIGNKEVAVDEISLVNVADGKLGTPGTPGR
DGRTPYVHTAWANNATGTDGFSLDSSINKLYIGIYTDFEPNDSTDPKKYKWAKVKGEKGE
KGDKGEPGQRGLDGLQGARGEQGLPGRNGADGRTQYTHIAYSNSADGTKDFSVSASDRAY
IGMYVDFNRADSNTPSDYNWTLVKGSDGANGVAGKAGTDGRTPYLHIAYATSNNGSQGFS
TTDSTNKTYIGTYTDYTQADSTDYRVYKWTLIKGADGTGISNVTNYYLATTVSTGITRTS
AGWTTTPQPITSDKRYLWNYRVELYTNGTSKTTEPTVIGVHGEKGERGLQGLQGLQGARG
EQGIPGPRGADGRTQYTHMAYADNATGGGFSQTNTDKAFVGVYIDFNPTDSRNPADYRWT
RWKGRDGANGVAGRAGADGRTPYLHIAYATSNNGSQGFSTTDSTNKTYIGTYTDYTQADS
TDPKKYKWAKVKGDKGEKGDKGERGLQGLQGLQGARGEQGIPGPRGADGRTQYTHMAYAD
NATGGGFSQTNTDKAFVGVYIDFNPTDSRNPADYRWTRWKGRDGANGVAGRAGADGRTPY
LHIAYATSNNGSQGFSTTDSTNKTYIGTYTDYTQADSTDPKKYKWAKVKGDKGEKGDKGE
RGLQGLQGLQGARGEQGIPGPRGADGRTQYTHMAYADNATGGGFSQTNTDKAFVGVYIDF
NPTDSRNPADYRWTRWKGRDGANGVAGRAGADGRTPYVHFAYSENADGSGLTMTDNGQRY
FGHYSDYEKPDSSDKTKYKWADRWAKVDGGYVNIYALSKNRSIGKSYHVSEFNMDVLSGN
ITLKAIGSDPYIGAVSSHPGIFIKQQGMKIPVIQGRSICITITNPLFRKNYISFFNSLGK
TVKTYKHYNTNKFLISSADLVGVEFIALRYGAGSSNIQIGTVLETKVKVEYGTVHSDWSP
APEDIESNINSKADQGLTQEQLNALNEKSQILEAEMKAKASMEAFSELEKAYNAFVKSNA
DSRKKSESDLVEAGRRIDLLTTQFGGLAELKTFIDTYMKSTNEGLIIGKNDASSTIKVSS
DRISMFSAGKEVMYISQGVINIDNGIFTASIQIGRFRTEQYHLNKDVNVIRYIGG
>orf02711
(SEQ ID NO: 1541)
MTKIMTFNGVDMSKFFRITDIIRPIGNKRSVSTDNAPLLGVNIQQVKIGEKEHIIKFDIK
TTNAIEMEQLKHDLAGILNVLEPVKITYGDEPDKYYMGLPVDEITPENLTRWFQRSELKI
IIPDGVAHSTTLKNFDIDTNETSAPDRIVFNLTNTGTEPAYPIIRIKHNSENGYIGVVNN
RAAFELGNREEADTEKYRDSETLIDYRGTNILKGFQNGTKGVAVTNDNKERLVGTLSTTS
MWGRNHIELSNRGTVEKNRNNAQSLTWAIPVDSSGEVGSLNDYLLWRQVFMAAVANQYGF
IKVTVSDTDGNFLYGVETYKRYQTLDCEYSFFTTDGKGGYKFIKWWYFTGTGAQVGKLDP
FSAEKGWSELKRNDDRVQVFFDGSHYDFIIPEIKDKKSAKIHITLGALRDWPLVSHMYVD
EFMYRKDFVTKSRDIPNRYPIGSNVVINSEDDSVYIDGISKVSEVVDGSHWPAIPPGKSQ
LELYFSRFVKKKPTVTIEFEERWI
>orf02714
(SEQ ID NO: 1542)
MADGKVTIVVDVDGNKVKVLNDELDKAAQKGDRGSDSLKKFAIGGAAFKLASKAVDLLTD
SLGGAIQRFDTLESYPRVMQAMGHSTEDVTRSTKKLAAGIEGLPTTLNEVVGTAQRLTSI
TGDINKSTDLTLALNNAFLASGSSSADASRGLQQFSQMLSAGKVDMQSWKTLQETMPYAL
QKTADSFGFAGQSAQNDFYSALKEGRITFNQFSKKLVELNGGVGGFAELAKSNSKGIQTS
FGNLKNAVVKGVANTIKALDDLTKAATGKTIAENFDALKVIINAAFGVIVNVIKASTPVF
QTLFSILGTGASVISSLTPVIISLVSALVAMRAANEAITATKNLINSWQTFKTTATGAIQ
IINLMTAAQATCGSVTKAQLVANLANNGALTASNLLYGVLTGSISLQTAATIAATAATTA
FKAALTALTGPIGLVVAGVGLAVGALVGLWQWLTAESEETKRLKSEQEELVKSTDQLTDS
VKQSAKERQKNLESVKGNTESYQKLADEIVQLSQKTNKTAADKKNLKKKIDALNASVSGL
NLVYDKNTDSLSHNNDQIKARISAMEAESTWETSQKNLLDIEQKRAEIGEQLKQIAEQRK
KWNEESNVSDSVRKERLQELNDKETELKNTQTELQTEYEKTSQVQQAASEAMAAAAENGS
NRQVISYEGMSKAQQKAVDDMRSKYNELLETTTNMFDQIQMKSAISVDEMIANLQKNQEA
VNNWATNLNTLAERGVNEGILAKLQAMGPQGGLYVQELVNASDEKLATLNEVFTQGGESA
MNGLTAGMDTGALGITDKIKGIVQSQVSSLQEEIAAADFPEKGKNIPEGVGDGIKAGAEI
ASEASKNMANDIKESFTSEMDINSPSRVFNEYGGFITTGLAEGVDKGTNQPVSSVTNLAN
QIKKPFDSLQSDFTYIGEMAMSGLNAGLWSGSGSVMATANSIAERVKATIKSALDIHSPS
RAMRDEVGRFIPQGIAVGIEADAGVVEKSMLRLKESMMIDTRPEIALGLNKKLGAQVTVK
QSSKQTIAEKIKVTMDKSSELLEKALDVAETAVRRPNEMYLNDGTLVARTGDKFAKYQSE
QLRRDNRMKGVLS
>orf02715
(SEQ ID NO: 1543)
MSMKLNDALITNFSIADKEYDIDLSFNKVLDVFEILKEDEMTRLEQAQLIVHLLTGQELY
DIKEVVDCWIYIKEHFLGIEKETVQYDLLGNPMPKAKGEEEQEKLIDFEQDAEYIYASFL
QAYGINLLKVQNELTWTEFKALLNALPDNTIMQQIIEIRAWKPEYGGDKNKMRKLQAKYS
LGKEGEDNG
>orf02717
(SEQ ID NO: 1544)
MTDIQIELKRTGFPVKIGEVELWFDTSQESLMCFYDMEEELKRRLVQYELDVVSANINNK
IERDGVTKEVVAGAIELEKKQLEIQYDLIFGDGTFDKLYSIYPDYNALNNALEQTAIMLH
DKLEEVAEQHKTVVKERASHYLNKGKVTPIKNNKKQKKNKKK
>orf02718
(SEQ ID NO: 1545)
MTRQKNALRGHFVAPYNGGTEPSTEDTWLELAKWISDVSDDTDEKTDDQAYYDGDGVEET
TVVSVKGAYTFEGTYDPDDKAQALIAGMKYKTGDDRKLWHKVVSSDRKKQWVGAATATEI
KAGSGAASDYEAFGCKLSYNSTPKETGIG
>orf02719
(SEQ ID NO: 1546)
MRENDFQNVLLKHIKTLNLPVEPRFDYFEDDKDDLVINQIPGGKVDREYMDGTQEVSLPF
EIAVKAKKNSVANDTIWLVTSELAKIDLVLPSDNNSYEYMGMEVSRPAMKGKDEQGYYYY
TIEIVAKIVIERNKQ
>orf02720
(SEQ ID NO: 1547)
MNIAIKVDLQKAKQKLSNESMTRGKIAVASKILLDNEQYIPLRGGELRASGRIVGQGDAV
VYGTVYARAQFYGSNGIVTFRRYTTPGTGKRWDQVATSKHAEEWARAFVKGMGL
>orf02721
(SEQ ID NO: 1548)
MTYLTQEEFDELDFDEVTDFEKLAKRAKIAIDLYTNGIYQKDIDFEKEIAYRKSAVKLAM
AFQIAYLDASGIMSADDKQLANSVSIGRTSISYSTSQSTLAGQRFNLSMDAENALRQAGF
SLVVGVAYDR
>orf02722
(SEQ ID NO: 1549)
MALYKATKNLFFEQLNMDVIVDDIIELDEDYAKEVNKKLKNAFPDVKNVLELVDKNGTLE
PEDAPSVDDASQATVED
>orf02723
(SEQ ID NO: 1550)
MPSNQNNAVRRYEKQYAGILETVFGVRAAFSNALAPIQILDGVQENSKAFSVKTNNTPVV
IGEYKTGENDGGFGDNSGAQSRFGGVTEVKYENTDVNYDYTLTIHEGLDRYTVNNDLNAA
VADRLKLQSEAQTRTVNKRIGKYLSDNATKTEALADFTDDKVKALFNKLSAFYTNNEVTA
PITVYLRSEFYNAIVDMASVTSAKGATISLDENGLPKYKGFTLEETPAQYFETGVIAIFS
PNGIIIPFVGISTARVIEAENFDGVKLQAAAKGGTYTLDDNKKAIYKVTGTIV
>orf02724
(SEQ ID NO: 1551)
MAFTTEELLNLGLTEEQAKSVFALRGKELNEDKSALETIKQERDSLKSQLQKAEEQVEHL
KSLENISAEQKDAIDKLQAEYDKYKNEAAAELAQTKKVSAISLALKDTNAFNPDKLMKFI
DVDAIQLDDNGKPQIDEVINGLKESDPYLFKAEESKPSPNILPQGNPAGEGTSDVDPFQA
IIDGYGK
>orf02727
(SEQ ID NO: 1552)
MKKKRKQITFNDQQFPLQMQGVGDIYEKLQIDIFDRMIKRLKERGSIDLMRNPYIWQLEK
LNDMHMLNEQNLKLISERTGIAERLLRDVIENEGLKVYKDTKQQLEEDLNKIPEGEISNG
VTDSLEAYSRQAVSDLNLINTTLPKSLQVAYKSIVEETVAQVVAGTKTSDVALHDTIMKW
QKNAFTGFVDKGGRHWKADSYARAIIKSTTYKVYNEMRTRPAEELGVDTFYYSMKAMARP
ACSPLQGQIVTKGTGREIDGITIYSLLDYGYGTAAGCLGIHCGHYLTPFIVGVHELPNLP
DYLKNLTPEQAEENARIEAGQRGLERLIKTHKERLHYAHTLQDDKMIQAERLKVRGYQTK
IRNLINQHDFLTRDYRREKLYIS
>orf02728
(SEQ ID NO: 1553)
MSLFQKVKDFFSRGRYYMQTSNLNSILEHPKIAVTQEEYDRIKRNLVYYQSKWDDVQYKN
TDGDIKSRPMNHLPIARTASKKIASLVYNEQATITTKNEILQKFLDDMLTNDRFNKNFER
YLESCLALGGLAMRPYIDGDKVRVAFIQAPVFFPLESNTQDVSSAAILTKTIKSEGRKNV
YYTLVEFHEWVTADGQETGSTNDKKYYRITNELYRSDVNDVLGQRVNLSELDKYKNLEPV
TVFENLSRPLFTYLKTPGMNNKDINSPLGLSIFDNAKTTIDFINRSYDEFMWEVRMGQRR
VIVPEHLTQRQYQRPDGTIDFRPRFDVEQNVYMQIGGSSMDAGGITDLTSPIRANDYILA
ISEGLKLFEMQIGVSSGMFTFDGQGMKTATEIVSENSDTYQMRSSIVALVEQSIKELCVS
MCELGKAVGVYSGEIPELDDISVNLDDGVFTDRHAELDYWAKMVAAGFSTKKRAIGKTLN
ISGVEAEKELNAINSELLPMNDAELAIYGMHDQNEEKADDKG
>orf02730
(SEQ ID NO: 1554)
MTFNVQKNINPHFKSVWISSLPYNVLKGGRNSFKSSVIVLKLAYMMIRYIIAGEAANIVV
IRKVANTIRDSVFNKVWWALNLFGIAEQFTKTVSPFKIVHKTTGSTFYFYGQDDFQKLKS
NDIGNIIAVWYEEAAEFNDQEDFDQSNVTFMRQKHPRAKFVQFFWSYNPPRNPYSWINEW
FESIKTNKNYLAHSSTYLDDELGFVTEQMLEDIERIKENDYDYYRYLYLGEAVGLGNNVY
NMSMFHAIDALPSDDKLIGISFALDGGHQQSATACCAFGITAKGKVILLDTWYYSPAGQV
VKKAPSQLSKEIYAYMRSVIEKYRVQALQYTIDSAEGALRNQMFLDFGLKWHPVAKLRKV
TMIDSFQSLLAQGRFYYLNTENNKIFIEEHKMYRWDEKTIKSDNPSVIKEDDHTCDTTQY
FVLDNAKLLGLRVGNV
>orf02731
(SEQ ID NO: 1555)
LPRDGTKNLKPVTERTKDEARAISSKGGKASGIARRKKADLKKAFETLLSLDVTDSKIKK
QLEEMGMAGNNEALLAFATFQQAVKGNQKATENIIKLTNTKDKYDIQEQKERIKALKYEN
RERAEAEKGSSETIEIVDAWAEDVRGATDDL
>orf02732
(SEQ ID NO: 1556)
VAQKLTKLKDIFKHVSSIDLGKEILFEDLELYNKETETSKQYQSIEEAENDLYLMEKVNK
INFTLGGGRGANFEKGKDGKYPGFRGAGGARDSGSSKALHPASLNNQGRFSSVEGAIQGF
IKKHGGSRTEYSTAVDSQGFAHNYVHGGKNSVQILPISGGFTAIHNHPNGSNFSSTDLHS
FAALKGMNTLVATNSSKAYRITKGANFDAKGFDKAVSKSRFTTKDYNKGADLWLKKNAKK
YGYTYSYE
>orf02735
(SEQ ID NO: 1557)
MLQIEYVDIKSIKPYHKNARHNDGEATEKVAASIKAFGFQQPILVDDNNIIITGHTRLKA
ALSLGIDTIPIAHAVNLTDEQIKAYRLADNRVAEYSTWDSELLNIELSQFETIDMAQFGF
ELSVTGFNFGNEEEQQEETENEEEDAEDFHRDTTINQYNLFNYDDTRVEGFYNMPKIEGV
DHIPKDFQGFNYVLNKPDYSSCVHFFLDDYQFERIWQRPDFYIEKLLEFDSALAPDFSLY
LDMPIAMQVWNIYRSRLIGQIMQDYGLTVIPTVSWASEESFDFCFDGLPKNSTLAISTIG
VKQNKEQFEVWKNGVTEMIKRLTPKRIVVYGGKVEYDYKDIEVVYFENATTERMKESGTK
TN
>orf02736
(SEQ ID NO: 1558)
MNVIGACQKILFYSPTQAYVLLNAWFNDYFRATYTELLENAILDK
>orf02738
(SEQ ID NO: 1559)
MNPEIIDNINKPSHYQGANGLEAIDVVHNFVGSLSGASAFFWGNAIKYMLRFQKKNGLED
LKKARKNLDWLIEEMDKEGR
>orf02739
(SEQ ID NO: 1560)
MFFQDSEIEEFELNDTLRNDYITAYPDEIELMQSTGLKDKNGKEIFEGDIVRTTRFLGRA
DEIGGFYEYEKDYVGVVKVLEGSWVIDTGSVAVRLWSEIDESEVLGNIYENLEFLEVNE
>orf02740
(SEQ ID NO: 1561)
MEDEQNILETQLILGKQVLEIVLDLLKDDSKIGVVLPLNINDREFTITVEKEVTDRD
>orf02741
(SEQ ID NO: 1562)
MTQTLEEGMKNQSKCIKIPMEIRPFDVGYRIVNKHGQALALKNGASIFALPSLAEKAIKK
EFGKNDPDFDIEKHFVEEVAIVNLSKFHSYFEEVE
>orf02742
(SEQ ID NO: 1563)
MNIKALIKKYEELWNEHSPFYEPVPYTSMVELFLKELKQLDEPEKVKVPRFVAEWIEEAR
KACKDVVELFEFDFTNDEVRKWFMQERPFDLVARAWLDGYEVEEEKRYLVTLKNRQPLVK
SQSGSTLYFSQDITARNYKGTQKELEDANFGWVFDCEGIEIEEVE
>orf02743
(SEQ ID NO: 1564)
MMEELKQKVNEVYNWTVEDGKPQPPKQDLPQAVKERVDYFWEMAEDGMTFMGAMECIFAD
EKPTDYDLGATKDWLPKSKEFDDWIGYAPSMAQVVIAVYLIYRGN
>orf02744
(SEQ ID NO: 1565)
VIEVNIKFDNFEAHGFYQDDTKLGKIRDAIISQMNNGHVVVLGEDRGILLNPKVIKSVQF
KVVEDNQI
>orf02745
(SEQ ID NO: 1566)
LYPTVKAIIDGMTDAGIWTDDNHKVIKKLSFVYGGLSEEKGHYRLEFDIEEV
>orf02746
(SEQ ID NO: 1567)
MTTENLKSALEYAVELNEHGLEILTAADGTEYYDANKFNLKELDPKRYPKTLELSTLTSL
VDYLKTDLNNLKNQRLIVAVEKNDEVCVWSENDEIEHRTLLVDVKARIPELSFGRFLSLE
QFNIMLQSNFIDDNDRGTLLEFASALKIENGAEIEDNGVSQVATVKTGVASLAKGKAPNP
VTLRPYRTFSEVEQPASLFVFRIDKQANMALFEADGKRWVADAVGNIASYLKEQLADQKH
ITVLA
>orf02747
(SEQ ID NO: 1568)
MDKKLIGLDLTHIADGGLQEKLDKELEKVFDNILDLNTDAKAKRKVTITLTMSANEERTV
VDTTMEVKSKFAPQNGVATTILIGRDFDTGQVHANELKSTVPGQMYFDENGEILTDIGQP
VAEIEQQAETKSDIIDFNKKKVGN
>orf02749
(SEQ ID NO: 1569)
MESAGHECIGFCEIDKFARASYKAIHDTKGEIELHDITTVSDDTIRGIGHVDVICGGFPC
QAFSIAGNRRGFEDTRGTLFFEIARFASILKPRYLFLENVKGLLNHDRGNTFEVILSALD
ELGYDVEWQVLNSKNFGVPQNRERVFIIGHLRGGSGRKVFPLSGDGAAITCEQPKINKVG
NTRKKGKSQSGDVVSIDSLAPTLCSTTTQKDPLKVLIENEIKQFGVLQPNYNQSGVVYEI
DGISPTIRAYQGGNLEPKIRVKEATKQGYQEAEIGDSVNLSHPNSKTRRGRVGKQIANTL
LTGESQGVVEPDFRIRKLTPRECWRLQGFPDWAFDKAQEVNSNSQLYKQAGNSVTVNVIS
AIAQGLGGN
>orf02751
(SEQ ID NO: 1570)
MINNVVLVGRLTRDAELRYTQSNIAVATFTLAVNRPFKNEAGEREADFINCVIWRQLAEN
LANWAKKGSLIGVTGVIQTRSYDNQQGQRVYVTEVVASNFQLLESRNSQQNNQGHQDHHG
GYQQQGYSNQGSSFQNGNSYGQQGSFVEGNTTNLVPDFTRDNNPFGRPTNPLDISDDDLP
F
>orf02752
(SEQ ID NO: 1571)
MRCFYVSGKIADLDLGSEINAENSFMAAIEFVKRYTDLLKFGSNEIKVSEVEEVQNDK
>orf02754
(SEQ ID NO: 1572)
MVKDVTNSLTEIKVDFQPAVINVDREAIEAQVAAAIAQYSGREVTVDNYKEVYEERTRFN
KLIGGLDTQRKDFNRQINEPAKDFDKWVKEKVIKPIEAVTDAMSAGLNAIDEHERLMRVD
VVRATFEDKCMVAGIEKSTFADKYDEYSLKKYFKTGKYELKKTTLDEMDGLVLSEFDALE
EYKANKQAIQEQAQEYDLPADSYIRHLEDGKSLVDILKMMKTDRDAEIARKEQKEIQEKA
KAERLEEIAQSAKKNANANIKAYDAETGEILEQGTITPEPQNNAREVAKFEPSEPLVKLV
RLELHGGLEQWENTQEYFEDNFIGFETLED
>orf02756
(SEQ ID NO: 1573)
MADLTFAELQRKMQIEKQTKQGVKYPFRTAEDINNKFKSLDSGWSVSFPEDDIIQKGDKL
YYKAVAVVKRESDGTIEKAIGWAREEDVPIFHTQKGDVKQMQDPQWTGAVGSYARKYALQ
GLFAIGGEDVDEYPVEESQEQGQNNQQQKPNNQQAQGQNQVRYIDNTQYQEINDLINDIA
KIKGMPFDTLANYVLSEKLKGLQDFHRVQVGDYEVLKNYLTEQLAKAKAKAKRGN
>orf02757
(SEQ ID NO: 1574)
MPNWAEGTLKLRGRRENVASALKEMLLGNKGATLEEEYDGTLLIFKNEYDYFYINGTRRA
FISSKDIEIWLDDDFVIIELEDFEQAWAALADNYTEISSKFDVDIKIFTFEMGMEFTQEI
EISKGEIIKNIVNENFTNYSWDVPFSRLGG
>orf02758
(SEQ ID NO: 1575)
MKKIATAMNVSVSDLFTQDTPIKKNRHSTPVNPKIYKEFIDNVNQYQRLIGATYEKISNI
IGKSNSYIYDVIDKQRKSTLSIKSNASLTKGSMILRQEIEKIESGKNRIPSKLTYQTIDR
DSEVVFRFNGLIKSVNELSDKELQIIVSIFDALKIPAKISNIEIRETNVFGGGK
>orf02760
(SEQ ID NO: 1576)
MKKLPSQQKYLRNDGQLVTIKGFDAYLQYRGSQSWKKEMAKTVKMTR
>orf02761
(SEQ ID NO: 1577)
MPDITNGREKVNDFLKDKGIKKTSLAIAYGFKRQEVTNILSGTTKGPRANSFILQVIEDY
GIE
>orf02762
(SEQ ID NO: 1578)
MFETFEKIKSLAKKQGISLNTLEDRVGLGKNYIYSLKNKKTPSAEHISKIADYFNVSTDY
LLGRTDNPTIANKKEQFFFEGKEVDVEELASTAMRFNGKPLTEEDKKAIQNIIEIYLRKQ
>orf02763
(SEQ ID NO: 1579)
MTEKEFSQNLGIDIEIFEDGLFPDEAFYIPALKTMFLSDAISDEKRVQVALHEIGHRNHA
PDTYQLFREKCELEANRNMIHHLMKAELDIAEDATTFNYLVFMEKYNLKTIADEIMVKEE
YLALLN
>orf02764
(SEQ ID NO: 1580)
MNIIAIIIIVIFVGGVIGAVIDNQKKSPEQRERELETFRANQEKKKQEKKQNIITCPNCK
SKDVTFLQQDKKAFSVGKAVGGAVLTGGVGALAGFAGKKGNKQWHCQNCGNFFETK
>orf02765
(SEQ ID NO: 1581)
MWMEELSNGKYKFFERYKDPYTEKLKKVSVTMEKKTPQARNQAAILLQEKINKKLSTKQV
ESITFEEIYNLFYKSWAQTVKESTKHNCKSVDKKMKEVIPSDTILANLDRRFLQEAIEKI
IESNGYITAKKVRHRLRGIFNYAVQYSYIENNEVDYTTIPQKPKTLEELEKKRNNFLTMQ
EIKALVDVLNRREYHQKYADMVLVLTLTGMRYGELTALQLKNIDFENNKIEITGNFDSVN
KIKTLPKTTNSIRTIKVSESVIEAIQRQIVRLSERFQPLSSDDYIFCFEKWNQPTTIACF
IQILKKYGKQAKIEKNLSSHIFRHSHISFLAESGLPIKSIMDRVGHSNAKMTLEIYSHTT
EDMEDKLVNKLDTIF
>orf02777
(SEQ ID NO: 1582)
LHPFTRNITCDRYILALFSNLVNFIHVDNPTFCTLNVKVSSLQEFEEDIFHILTYITSLR
QSCRIRNRKRYIQALSQGLSKESFP
>orf02778
(SEQ ID NO: 1583)
MIINRHCQGTLGTILTDYIVVQDMEEFDWFWYLRQVCQDFLNQFFSNNFLS
>orf02786
(SEQ ID NO: 1584)
LKTKIGLASICLLGLATSHVAANETEVAKTSQDTTTASSSSEQNQSSNKTQTSAEVQTNA
AAHWDGDYYVKDDGSKAQSEWIFDNYYKAWFYINSDGRYSQNEWHGNYYLKSGGYMAQNE
WIYDSNYKSWFYLKSDGAYAHQEWQLIGNKWYYFKKWGYMAKSQWQGSYFLNGQGAMMQN
EWLYDPAYSAYFYLKSDGTYANQEWQKVGGKWYYFKKWGYMARNEWQGNYYLTGSGAMAT
DEVIMDGARYIFAASGELKEKKDLNVGWVHRDGKRYFFNNREEQVGTEHAKKIIDISEHN
GRINDWKKVIDENEVDGVIVRLGYSGKEDKELAHNIKELNRLGIPYGVYLYTYAENETDA
ENDAKQTIELIKKYNMNLSYPIYYDVENWEYVNKSKRAPSDTDTWVKIINKYMDTMKQAG
YQNVYVYSYRSLLQTRLKHPDILKHVNWVAAYTNALEWENPYYSGEKGWQYTSSEYMKGI
QGRVDVSVWY
>orf02791
(SEQ ID NO: 1585)
MHKNFVVVVTDFFTAVQFIQFNKEGTTCHNTTKFFNHLDSCLNSSTCRQKVIYNKNTLTW
LNGIRVHSQGIDTVLFFIVSRNNFAWQFTWLTNRRKTNSQLKGNWTTHDKSTSFRSHDHV
DFLVSSILNDFTNSVAISISISHQRTNITEGNAFLWIIFNCCNVIF
>orf02795
(SEQ ID NO: 1586)
MEINEQTRKLAVSYSKYSFEVADKTDEVSNHTYGKATLTWFEEIFEEYKEHHNIDV
>orf02801
(SEQ ID NO: 1587)
LHKTLENIGEFEEDNLYYSSMTKAETRISFPIFSLILHYI
>orf02803
(SEQ ID NO: 1588)
MLNRQVCFCFVNHISPLNVVIWENLSLEELLYAICICFITHKIAKQTSLTIDNAGIAMNN
IR
>orf02808
(SEQ ID NO: 1589)
LNSRFFYTDFFKGRQAKGCSFSCTSLSLTDNILAFKGQRNSLFLDRTSFYKTSFFNFC
>orf02821
(SEQ ID NO: 1590)
MRFLADQDRIQHHRYSWALFDKVQGLLSHTDSREKTNLNSPKFHIT
>orf02822
(SEQ ID NO: 1591)
MLKNGIISWKDFKSFFCQGCQTSHCYKPMQAVQGIGSQIS
>orf02825
(SEQ ID NO: 1592)
VTAHRIFGTSSIHNKLIGLAMFGITAMKIICHKLNRNHINIFRRLGIQGKTEFLLIHLIR
QVKMNDLSQGMNPTICPTSTVNSNGLPFI
>orf02829
(SEQ ID NO: 1593)
MLARSKNCFMKSLSIFLLIFYFFDSYQISKKRRSLIGL
>orf02840
(SEQ ID NO: 1594)
LEVCIHHHHQISCRILQACIKGCFFAKISRERNIMDCRILLPIGL
>orf02847
(SEQ ID NO: 1595)
VDRTDEVSSKHCFEVVDRTDEVSNHTHGKATLTWFELDFRRV
>orf02893
(SEQ ID NO: 1596)
MIAEFIDGLQKFHFLQNALITAIVVGIVAGAVGCFIILRGMSLMGDAISHAVLPGVALSF
ILGLDFFIGAIVFGLLAAIIITYIKGNSIIKSDTAIGITFSSFLALGIILIGVAKSSTDL
FHILFGNILAVQDTDMFITMGVGAAILLLIWIFFKQLLITSFDELLAKAMGMPVNFYHYL
LMVLLTLVSVTAMQSVGTILIVAMLITPAATAYLYANSLKSMIFLSSTFGATASVLGLFI
GYSFNVAAGSSIVLTAASFFLISFFIAPKQRYLKLKNKHLLK
>orf02913
(SEQ ID NO: 1597)
MYEEPEVAPVHPTGPTPATETVDSAPGFEAPQESVTIL
>orf02945
(SEQ ID NO: 1598)
MGNNGQFTFGYRHDFFQNQLAIFNALVDTFTRRTIDIKTLNTFINEVLNQGTRTLWTYFS
LLIITCVEGWNDTFVFFQI
>orf02948
(SEQ ID NO: 1599)
LTKIFGWILRIAVLAADVYGNFANNIAVAWDAHDKIPNNGRINF
>orf02974
(SEQ ID NO: 1600)
LSTRNKYCKNLIIFESTFNILDIVKKDLKLNSKLEKDLKY
>orf02976
(SEQ ID NO: 1601)
MSYFRNRDIDIERISMNRSVQERKCRYSIRKLSVGAVSMIVGAVVFGTSPVLAQEGASEQ
PLANETQLSGESSTLTDTEKSQPSSETELSGNKQEQERKDKQEEKIPRDYYARDLENVET
VIEKEDVETNASNGQRVDLSSELDKLKKLENATVHMEFKPDAKAPAFYNLFSVSSATKKD
EYFTMAVYNNTATLEGRGSDGKQFYGNYNDAPLKVKPGQWNSVTFTVEKPTPELPKGRVR
LYVNGVLSRTSLKSGNFIKDMPDVTHVQIGATKRANNTVWGSNLQIRNLTVYNRALTPEE
VQKRSQLFKRSDLEKKLPEGAVLTEKTDIFESGRNGKPNKDGIKSYRIPALLKTDKGTLI
AGADERRLHSSDWGDIGMVIRRSEDNGKTWGDRVTITNLRDNPKAFDPSIGSPVNIDMVL
VQDPETKRIFSIYDMFPEGKGIFGMSSQKEEAYKKIDGKTYQILYREGEKGAYTIRENGT
VYTPDGKATDYRVVVDPVKPAYSDKGDLYKGNQLLGNIYFTTNKTSPFRIAKDSYLWMSY
SDDDGKTWSAPQDITPMVKADWMKFLGVGPGTGIVLRNGPHKGRILIPVYTTNNVSHLNG
SQSSRVIYSDDHGKTWHAGEAVNDNRQVDGQKIHSSTMNNKRAQNTESTVVQLNNGDVKL
FMRGLTGDLQVATSKDGGVTWEKDIKRYPQVKDVYVQMSAIHTMHNGKEYIILSNAGGPN
RENGMVHLARVEENGELTWLKHNPIQKGEFAYNSLQELGNGEYGILYEHTEKGQNAYTLS
FRKFNWEFLSKDLISPTEAKVKRTREMGKGEMGKGVIGLEFDSEVLVNKAPTLQLANGKT
ATFLTQYDSKTLLFAVDKEDIGQEIIGIAKGSIESMHNLPVNLAGARVPGGVNGSKAAVH
EVPEFTGGVNGTEPAVHEIAEYKGSDSLVTLTTKEDYTYKAPLAQQALPETGNKESDLLA
SLGLTAFFLGLFTLGKKREQ
>orf02978
(SEQ ID NO: 1602)
VDKTDKVSSKHRFEVADRTDEVSSKHRFEVADRTDEVSSKHRFEVADRTDEVSSKHRFEV
ADRTDEVSSKHRFEVADRTDEVSSKHRFEVADRTDEVSNIYTARRS
>orf02989
(SEQ ID NO: 1603)
MSCNCAFYRSQFFDVNSVSNYHSHQKELRFPNSILFTYFVKVT
>orf03007
(SEQ ID NO: 1604)
MTVKHRIVNSNGEARSGESLTTCGTRSTHGNWATVGYVYAADMARQNWWDLSAAISGSWS
PN
>orf03009
(SEQ ID NO: 1605)
MRWDYGQIFKEIRKSKGLTQQDVCGQVIHRTTLTNIEHGKVIPSFENMVFLLEQIDMSLA
EFKYICNEYHPSKRRDIIVESQNPSTFQDTRKMVELTEKCQKYLKTHHDVPIQNIYRHTK
IVTELRTKGFKNNHVLKDLYEEIWDYLEPMDTWYISDLKLLGTILFFFPSENLPLLIDRI
MKTIEKYKYFRETKAFLSSFLANLSTVYFQHHLFKECETITLQLLVLAEELKIYDILGFS
QVRLGILQHNSDLIDKGITLLRLTKEEALVKILEKEINDFSNL
>orf03014
(SEQ ID NO: 1606)
VGLIKLTSYVFVCISNSFLTRHDKNDNICFFHGNFCLVLDLFHERSIDIINSSCINHAKR
TIEPLTRCINTVTCHSFDIFYNGDSLTSDPIK
>orf03016
(SEQ ID NO: 1607)
LSSKSCIDRTNQETFHTLGLEGVGMKSGSLFCSVQISDKEKENSRLANGFLRYQFIQGIF
LLLTSYHNHRVGLEILPR
>orf03049
(SEQ ID NO: 1608)
MTESYTWVEADRATLSRYRHGQGHLTDQFFSFKVQRPAAKTLIASISTGKGMGPSFDGTP
VITSGNQNRINTIKNSFIMSSSSVRISLRKLTSQRNFLRNLSSLILLAAQVAKGDATACS
HQRISRVVGQDSHETLSLTEFF
>orf03050
(SEQ ID NO: 1609)
MNINNEKVWFAFYLLDMQITRPTPTFNDRRIGLIGKLQELRFLAGNLLLR
>orf03051
(SEQ ID NO: 1610)
LIKGYLPNHLALMDLCSKTTCTLDDFAGIAGRRNHRGFFCHIGNGVFLTVDKYLRNQRIR
QRKSSHHILTQLVCHSHTHLFILLQTSLSLRTKERLSF
>orf03057
(SEQ ID NO: 1611)
LIKLTDRNFSDILIKCLIKCFTNLLSNQLMLLPSTLKL
>orf03060
(SEQ ID NO: 1612)
LFKGGVTISRTPLSSEDTVMIDATEVQINCPKKTISE
>orf03062
(SEQ ID NO: 1613)
MIQSENHCSASHSNRDYQSQHDNQGRTCQCFIIVPCHKKGSCSVGEITWNQRCQNGQDKD
HSRCLIKNT
>orf03069
(SEQ ID NO: 1614)
MIARQLMVFFSTNQADTRITNMSIDSLIINNSKDFQSSSHASVSFILTKLVNLLIFNF
>orf03070
(SEQ ID NO: 1615)
MGEPFTHFIDCIDLGINPSYTQVCDRHFTSDIPCTMTSHPIS
>orf03077
(SEQ ID NO: 1616)
LSSDSHFIGIKAFVILILGKSNSIVLRIVGLYQDLTCFFSPTCSTCHLSQELEGSLRRTE
IRQIQGRIRI
>orf03078
(SEQ ID NO: 1617)
MAVHSLGIHMQGQRNIAVGTSIHRPTLPTHDKARITTAIEHENHLLFFNQTVLDSL
>orf03079
(SEQ ID NO: 1618)
MVTGIAVLLISHFMLFINNHDTQIFQRSKDSRSGTNNNLGIATLHLAPFIILFTIG
>orf03080
(SEQ ID NO: 1619)
VKNGYLVPKTCYKTLGHLRSQGNLRYQQNSCLALIQGTLDNLQVNLGLPTSCNPLK
>orf03081
(SEQ ID NO: 1620)
MVNLIPRLGLDLLLIDCLIFQTKQAFSSQTHHFSLLGKV
>orf03082
(SEQ ID NO: 1621)
LGLQTKNNPLNQAIPLTKRHMNPHPNFQHSLKFLRNPVTIGLVRLHQGHIYDNLS
>orf03085
(SEQ ID NO: 1622)
LGNHFCTICSTTYQAILQFIQIWWCQEDKDSIWNLFLDLKSTLNFNFKENIDSLVQGFID
IGQRSSIVVADIFCVFQHLSLTNQLFKFFTSTEEIVNTVHFSRTLCACRHRYRILKLVFR
TLKNLSSNRSFSNP
>orf03092
(SEQ ID NO: 1623)
MKFKNYLFDLYPYFPKGFSLDNREDPDVCSKALYDDLCKMFFDDDSKEKLKITSVCNKCQ
NYGNRDYYTLFIDKDKYLLSSDYIGASIYWAQEAGLNDRIILDHLSISRTIGGHILFPRG
GKLETVNQARGGEKGYYDRFDLTLYAIKEWFVENKNTKIGYAIENYHEWFELFSGDDNCK
NGFENFVEFFKLEGFIYEQNKIIDLIKSDLENNQVVFLDKEDILIASTEEEYIRYMKNLN
IIILERTKKILL
>orf03093
(SEQ ID NO: 1624)
MELSIQLIHDLNTHTTHSTAKMLHNVKAIKNDFSIRE
>orf03096
(SEQ ID NO: 1625)
LKAKLFSQVIVRAKHIHSNNFDATSDSSIVAKKVISNDSLFSSLKNSDDIEIVKILRNEA
HLSLCKSILIPRHNLRKTRKIMFKVKIKEQTRKLAAGCT
>orf03113
(SEQ ID NO: 1626)
VGCSYICHELVANHDHFLFVIVEFLHGTVNTKCEGLQGPVNVINPKFLNCSLNAFFGVI
>orf03114
(SEQ ID NO: 1627)
LLHLWRSIRVVPSNEGIIQIDQNSLDSLRLQAWDCQIIDCFHSKIWYIIFNRHSGSFS
>orf03118
(SEQ ID NO: 1628)
VKGLLLATKLCRTNSHTDNLTRYSNRSICQNDLISHIQLTFKEDEKAIDDIRQKALGSHT
NRYPSNTSSSQQTRNWQT
>orf03120
(SEQ ID NO: 1629)
LWGILGLTLPNLSGIGLLGDLFVGGLKAVAPILVFALVANALSQHQKGQDSNMKTVVFLY
IL
>orf03121
(SEQ ID NO: 1630)
MIGTFAAALVAVLASFIVPIEITLNSANTEIAPPDGIGQVLSNLLLKLVDSPVNALLTAN
YIGILSWAVIFGIAMREASKNSKELLKTIADVTSKIVEWIINLTPFGILGLVFKTISDKG
VGSLANYGILLVLLVTTMLFVAPVVNPLIAFFFMRRNPYPLVWNCLRVSGVTAFFTRSSA
ANIPVNMKLCHDLGLNPDTYSVSIPLGSTINMAGVAITINLLTLVTVNTLGIPVDFATAF
VLSVVAAISACGASGIAGGSLLLIPVACSLFGISNDIAIQIVGVGFVIGVIQDSCETALN
SSTDVLFTAVAEYAATRKK
>orf03124
(SEQ ID NO: 1631)
MKIKEQTRKLAAGCSKHGFEVVDRTDEVSSKHRFEVVDRTDEVSSKHRFEVVDRTDEVSS
KHCFEVVDRTDEVSSKHCFEVVDRTDEVSSKHGFEVVDRTDEVSSKHGFEVVDRTDEVSS
KHSFEVVDRTDEVSSKHGFEVVDRTDEVSSKHGFEVVDRTDEVSSKHGFEVVDRTDEVSS
KHSFEVVDRTDEVSNIYTAR
>orf03145
(SEQ ID NO: 1632)
MKDLISVIVPVYNVEPFISSCLDSLSKQIYQNFEVLLVNDGSTDNSGAICREYADRDSRF
HYFEKENAGVADARNFGIERSKGDYITFVDSDDWVTEEYLSILIETLKEQHSEIVVSTYS
TYNESDGLFYIHVFDSDYYVKNYNSKLLMEELPLLERYDMSFLTSWGILFKRELFQEVQF
PFGRVCEYIGTNYKLFMQVEKVTYINKVLYWYRVGKEGLSNSYSPKMMRDDCDFRLERIA
VLALKGYDVSKYLDQMKFYLKYRHDIAIQRELKENVETRHLEMLDYLLNGNKYN
>orf03148
(SEQ ID NO: 1633)
LNVRGGAYITFVDSDDWLEHDALDRLYGALKKENADISIGRYNSYDETRYVYMTYVTDPD
DSLEVIEGKAIMDREGVEEVRNGNWTVAVLKLFKRELLQDLPFPIGKIAEDTYWTWKVLL
RASRIVYLNRCVYWYRVGLSDTLSNTWSEKRMYDEIGAREEKIAILASSDYDLTNHILIY
KNRLQRVIAKLEEQNMQFTEIYRRMMEKLSLLP
>orf03167
(SEQ ID NO: 1634)
VLAKAEAEALVDADSDADVLADTEAEALVDAEAEALVEADAEALVLAEAEALVDAEADAL
VEAEAEALVDADADALVDADSEALVDADSDALVLAEAEALVDADSEALVLADSDALVLAE
AEALVDAEADALVDAEADALVLAEAEALVDADSEALVDAETEALVDAEAEALVDAEADAL
VDADSDALVDADSDAEVLAEADALVDAETEALVEADSDAEVLAEADALVLAEAEALVDAE
ADALVDAETEALVEADSDAEVLAEADALVEADSEAEVLAEAEALVDADSDAEVLAEADAL
VDADSEALVDAEAEALVDAEADALVLAEAEALVDAEAEALVDAEAEALVLAEADALVDAE
ADALVDAEAEALVDAEAEALVDADSDALVDADSDAEVLAEADALVDADSEALVDAEAEAL
VDAEADALVLAEADVLALVDADSEADVLAEADALVDAEADALVDAEAEADVDADSDAEVL
AEADALVEAEALVLAEAEALVDAETDALVDAEAEALVDAEADALVLAEADVLALVDADSE
ADVLAEADALVDAEADALVDAEADALVDAEADALVLAEADALVLAEADALVLAEADALVL
AEADALVDADSEADVLAEADALVDAEADALVDAEAEALVLAEAEALVLAEADALVDAEAD
ALVLAEADALVDAEAEALVLAEAEALVLAEADALVDAEADALVLADSDALVDAEADALVD
AEADALVLAEADALVDADSEALVDAEADALVDAEAEALVLAEAEALVLAEAEALVDAEAD
ALVDADSDALVDAEAEALVLAEALVLAEADALVDAEADALVLAEADALVDAEADALVLAE
AEADVDADSEADVLAEAEALVDAEAEALVLAEAEALVNAEADVLAEADALVDADSEALVL
AEADALVLAEAEALVDAEAEALVDAEADALVLAEADALVLAEAEADVDADSDALVLAEAE
ALVDAETEALVDAEAEALVLAEAEALVLAEAEALVDAEADALVLAEADALVDADSEALVD
AETEALVDAEAEALVLAEAEALVLAEADALVDAEADALVDAEAEALVDAEADALVLAEAD
ALVDAEAEALVDAEADALVLAEAEALVDADSDALVDAEADALVDAEAEALVDADSDADVL
ADTEAEALVDAEADALVLVDADVLALVDADVLADVLALVDADVLAEAEALVLAEAEALVD
AEAEALVDADSDAEVLAEADALVLAEADALVDAEAEALVDAEAEALVDAEAEALVDADSD
ADVLAEADALVDAEADALVLAEADALVLAEADALVDADSEALVDAEAEALVDAEAEALVD
AEAEALVDAEAEALVDAEAEADVDAEAEALVDADAEALVLAEADALVDADSDADVLAEAE
ALVDAEADALIDADSEADVLAEAEALVDAEADALVDAEAEALVLADAEALVDAEADALVD
AEADALVDAEADALVDAEAEALVDAEADALVLAEAEALVDAEADALVDADSDAEVLVLAE
AEALVDAEADALVDADSDAEILAEADALVDAEAEALVLADSDALVNAEADVLAEADALVD
ADSEALVLAEADALVLAEAEALVDAEAEALVDAEADALVLAEADALVLAEAEADVDADSD
ALVLAEAEALVDAETEALVDAEAEALVLAEAEALVLAEAEALVDAEADALVLAEADALVD
ADSEALVDAETEALVDAEAEALVLAEADALVDAEAEALVDADSDADVLADTEAEALVDAE
ADALVLVDADVLALVDADVLADVLALVDADVLAEAEALVLAEAEALVDAEAEALVDADSD
AEVLAEADALVLAEADALVDAEAEALVDAEAEALVDAEAEALVDADSDADVLAEADALVD
AEADALVLAEADALVLAEADALVDADSEALVDAEAEALVDAEAEALVDAEAEALVDAEAE
ALVDAEAEADVDAEAEALVDADAEALVLAEADALVDADSDADVLAEAEALVDAEADALID
ADSEADVLAEAEALVDAEADALVDAEAEALVLADAEALVDAEADALVDAEADALVDAEAD
ALVDAEAEALVDAEADALVLAEAEALVDAEADALVDADSDAEVLVLAEAEALVDAEADAL
VDADSDAEILAEADALVDAEAEALVLADSDALVNAEADVLAEADALVDADSEALVLAEAD
ALVLAEAEALVDAEAEALVDAEADALVLAEADALVLAEAEADVDADSDALVLAEAEALVD
AEADALVLAEADALVDADSEALVDAETEALVDAEAEALVLAEAEALVLAEADALVDAEAD
ALVDAEAEALVDAEADALVLAEADALVDAEAEALVDAEADALVLAEAEALVDADSDALVD
AEADALVDAEAEALVDADSDADVLADTEAEALVDAEADALVLVDADVLALVDADVLADVL
ALVDADVLAEAEALVLAEAEALVDAEAEALVDADSDAEVLAEADALVLAEADALVDAEAE
ALVDAEAEALVDAEAEALVDADSDADVLAEADALVDAEADALVLAEADALVLAEADALVD
ADSEALVDAEAEALVDAEAEALVDAEAEALVDAEAEALVDAEAEADVDAEAEALVDADAE
ALVLAEADALVDADSDADVLAEAEALVDAEADALIDADSEADVLAEAEALVDAEADALVD
AEAEALVLADAEALVDAEADALVDAEADALVDAEADALVDAEAEALVDAEADALVLAEAE
ALVDAEADALVDADSDAEVLVLAEAEALVDAEADALVDADSDAEILAEADALVDAEAEAL
VLADSDALVDADSEALVDAETEALVDAEADALVLAEAEALVDAEAEALVLAEAEALVLAE
ADALVDAEAEALVDAEADALVLAEAEALVDADSDALVDAEADALVDAEAEALVDADSDAD
VLADTEAEALVDAEADALVLVDADVLALVDADVLADVLALVDADVLAEAEALVLAEAEAL
VDAEAEALVDADSDAEVLAEADALVLAEADALVDAEAEALVDAEAEALVDAEAEALVDAD
SDADVLAEADALVDAEADALVLAEADALVLAEADALVDADSEALVDAEAEALVDAEAEAL
VDAEAEALVDAEAEALVDAEAEADVDAEAEALVDADAEALVLAEADALVDADSDADVLAE
AEALVDAEADALIDADSEADVLAEAEALVDAEADALVDAEAEALVLADAEALVDAEADAL
VDAEADALVDAEADALVDAEAEALVDAEADALVLAEAEALVDAEADALVDADSDAEVLVL
AEAEALVDAEADALVDADSDAEILAEADALVDAEAEALVLADSDALVDADSEALVDAETE
ALVDAEADALVLAEAEALVDAEADALVDAEAEALVDADSDAEILAEADALVDAEAEALVD
ADSDAEILAEAEALVLAEVDALVEADSDADVLALVDADVLALVDADVLADVLVLVDADVL
AEAEALVLAEADALVDAEAEALVDADSDAEVLAEADADALVDAEAEALVLADAEALVDAE
AEALVDAEADALVDAEADALVDAEAEALVLADAEALVDAEAEALVLAEADALVDADSDAL
VEADSDAEVLAEAEALVDAEADALVDAEAEALVLAEAEADVDADSEADVLAEADALVDAE
AEALVLAEADALVDAEADALVDAEADALVDAEAEALVLAEAEALVLAEADALVDAEADAL
VLADSDALVDAEADALVDAEADALVLAEADALVDADSEALVDAEADVLVDAEAEALVLAE
AEALVLAEAEALVDAEADALVDADSDALVDAEAEALVLAEALVLAEADALVDAEADALVL
AEAEALVDADSDAEVLAEAEALVDAEADALVDAEADALVLAEAEALVDAEADALVLADSD
ALVDAEAEALVDAEADALVLAETDALVDADSDAEVLAEAEALVDADVLADVLALVDADVL
ADVLALVDADVLADVLALVDADVLADVLALVDADVLAEVDALVDADVLADVLAEADALVD
ADSDAEAL
>orf03175
(SEQ ID NO: 1635)
VFEVVDKTDEVSSKHCFEVADRTDEVSLKHCFEVADRTDEVSLKHCFEVADRTDEVSNHT
YDKVKLTWFEEIFEEYHTKKPCSSR
>orf03178
(SEQ ID NO: 1636)
MYQDLLRKIAEEKPNYNQEEIQWLLDHLGDPSPEIRDDLVFTSFARGIQEELFTQEQFHF
IAEGVSSDGGLDKEIDKIGLPTLERSFRALIYATLLSDDANQQSIFYQRLKAEIRNVLLN
QGLHYLSKEKDTTGFSSQYGWVHSFAHGADLLKEVVCHPDFPKNRVHEVFDILGQLFKRM
SIRFTDDEDWRLARVIYEPILQGKLEQEQVASWIKTVDFPIEEREDFSKFSNFRSCLVEV
YVQLDQRNSLQDELKEAIQSFQY
>orf03181
(SEQ ID NO: 1637)
MGFKVSHFKIPSSHLSINVLRTIENFTEIGQGLLHISP
>orf03182
(SEQ ID NO: 1638)
VGFFDFGLTNSCRQVRQFTQTVQDFLVCYHQGIVKEGQGYAGICFKFHPSLGNIGKFVIA
IVRRLRHKSIVANMAHLNVDLFQFRKGLLEILKSVKIALVITAKLVDVFTSFLDCTQEIL
TVLV
>orf03190
(SEQ ID NO: 1639)
MNITYIVGNGLDLQYGLKTRYRDFYEFQNKVYISRTENEEKYSNFIYESLFSDKVNDYEN
WSDFELSIGKLTKDNDLISSSIEIKEKFIDDFSEVVDDLREYLRIQQEKNLEKGNAIDFI
STLDDMRTSLPVINQPAIDKKYNENPHQDDIVNIVTLNYTHVIDKLYNGSAKSFRNQLRA
NLYNFYIEPPIHAHGTVDVCTVLGVSDEIQISNSLEEQKESLIKSLVLKNYRENMDVKNS
DIIKNSDIIILYGVSLGETDRYIWSQIAERSISGSVPVIIYHYVPHFDPGNPIRAKRLYR
NVEDKFIQNSGIDLELEKKLRDNLIVVIGKTIFDLIER
>orf03191
(SEQ ID NO: 1640)
MNTLLTLRGKSFTQKSRNNGMGPITIPKKTIITLEHLKYLHFSLEETKTYWEKNNIIDGI
LISIYYNRIVAKSNRINGYFNVGGGNPFPNDTIVGAKFNDEKTKHIVTHYISRDALNKTI
TVLSKIIEVFEEHFDRAITCEMFSDSSTFASINFSEYGISKSKFQQYLRDSCFIENFGVE
HTTVSDIQNSIVTFYDVHTDIFRLLNKLNIDISEANIMNQTTVLLDEKNIELLLSKAPYL
VSMIVEDFSKLSVDDFSLDNNDLKINLPSPMNEPVVGVIDTLFDKRVYFNEWVEYHDFVS
PDISKDSQDYKHGTAVTSLIVDGANLNPNLDDGCGNFRVRHFGVSLQSGFNSFTIIKQIK
EIVSQNADIKVWNLSLGSNDEIRENFISAEGALLDEIQFENDVIFIIAGTNASVINGKRK
RIGAPADSLNSIIVNSVDFNNQSVSYSREGIVLSFFVKPDVSYYGGGNGDFINVCEPLGL
GRVAGTSFAAPFIARKMAYLIHIMGLSREEAKALLIDAAIPWNDKKTFTDLSLIGNGIVP
IKMDDILSTPDDEIKFIVSDISRAYDTYNYDFPVPISSESYPYVAKATMCYFPNCSRKQG
VDYTNTEMQLTFGRLKSDGIKSINKDNQHAEDTPGYVRENAARNIFRKWDNVKHIGESFT
SRKRAKAILNPSNPQWGMSIKTIERLKSGDGQGVRFGVVVTLKELNGVNRIEDFIQQAEL
RGWLVNRLQVEAQVDLFNSLNEEIEFE
>orf03192
(SEQ ID NO: 1641)
MKKSDVLDLIKYHYEGRETEFRNQSIAIARNFNKHGDTQIAQYIMGLMSQSDRFMPQIEN
PSEYLTPAKLDIGPLPLPLSIMNDLKGIINAVNHHIGINKFLFVGSPGTGKTESVKQVAR
LIGKELLVVDFSHLVDSKLGQTVKNLATLFNEINNLPFKQNYIILFDEIDSIVLDRVNQN
DLREMGRVTSAFLKELDRLSPEIVLIATTNLFENLDKAVTRRFDAIIDFDRYTDEDKVEV
ATIILNELLKQFKNVARDLKLFKKIINSANVIPNPGDLRNSIRTSLAFSDPSDPHDYQKR
LLRSLHNGRNLSISKLSKLGFTVREIEILTGISKSSVSRELSED
>orf03200
(SEQ ID NO: 1642)
MINSQVFEIRIFNSYYKDAIYYFKNINYSIFHIFSTHY
>orf03205
(SEQ ID NO: 1643)
VGHRFDPCRGHLNTTGKALEPRFFCLNKIFFKFFRKLA
>orf03206
(SEQ ID NO: 1644)
MGWKGTPPCLHPSNQDTTILIVQQCLRRIEVLAMINFLN
>orf03207
(SEQ ID NO: 1645)
VFGSYYRVIASIFFKEFWITEISSNQLIWQVCSSYNWILGNLFKVNPVI
>orf03208
(SEQ ID NO: 1646)
VLPSHQVLTFSMSPVHRSPNTIIWIELIKEMVFSTKINKSIWIIDPTNLS
>orf03219
(SEQ ID NO: 1647)
LLRFAIHSNLLFLKRFIFSIKDSAWRCLFISFFETFHFCFKNKSFYLNSFYH
>orf03230
(SEQ ID NO: 1648)
LIVSLKTKSRKAKDMAESIQGWLAQFLVNLFKSITFDCGKEFSKWKDISNHHDSESFFAN
LECPRQRCLNEHSNRLLRCHDLPKQTDFNEVSQEF
>orf03231
(SEQ ID NO: 1649)
VVEIIYFLIIIIASGLGSISGMGGGIIIKPLMDSFGYHSVSDIAFYSSFSVFIMAIISTT
KRFSQSKEIKWRLIFTVSFSSVLGGFLGHLIFQVLLSQLSVRLVSIVQMILLFVMLLVSF
VLTDFKKTYQFDKIGFYMICGLLLGLISSFLGIGGGPLNVSLLMVFFSISIKEATMYSLA
IIFFSQLSHLATIVVVTGLNQYHLAPVPVIFLASICGGVLGTVVSKVLPENWVRYCFKGM
LFFVMGMTLYNLFHIL
>orf03232
(SEQ ID NO: 165)
MMGTNSEEGFLDDFEGPQVAVSVKDFSIADTPVTNQEFAQFVKETGYKTLAERQEWSFVF
ILFVPEAEREGYPHPAGAPWWLQVSNACWKHPYGENSNLVGLEDHPVVHVALEDALAFCN
WSGMSLPTEAQWEYAARGGRQSEYPWGDTLLEGGYYHANTWQGRFPYENTALDGFIGTAP
VYEFLPNDFGLYQMIGNVWEWCRNPRYTLLASFNEDDYELPKYGIQDEEYAIRGGSFLCH
CSYCNRYRVAARNGCISTSTSSHLGFRCLKE
>orf03235
(SEQ ID NO: 1651)
MVQTKQPNIILIVVDQMRADALSLNSKDKLVSTPTLDMMASVGYNFENAYSPVPSCVPAR
AALLTGLDQDKSGRVGYQDEVPWNFTNTLPKVFKDMGYQTECIGKMHVFPSRQRLGFDHV
LLHDGYLHVDRKYDKAYGSQFDYASDYLAFLKGKVGYDVDLIDDGMDCNSWEARPWDKDE
KLHPTNWVVSESISFLQRRDPTVPFFLKMSFEKPHAPLNPPKYYFDIYMERLPQFLDLHI
GNWEVLEKQIPSIYALRGKLKEDDQRRMVAAYFGLITHIDHQISRFLTALKEFRHDKDTI
IWFVSDHGDQLGEHYLFRKGYPYQGSIHIPSFIYDPAGLIAGNRGTIKQLVKIQDIFPSL
VDLAGGTTTDELDGRSVKNLLFGQYEGWRTEFHGEHALGKDSSQYILTDQWKFIWFPVLN
HYQLFDMKKDPHEMNDLYPSEKYQPIVRQMKKKLVDFLRYREEGFVVDEELVPVELSKIT
PTLTKTGDSQS
>orf03237
(SEQ ID NO: 1652)
MNTMLDKMQEKLSPIAMKVGNQKFLVALRDSFVGTMPVIMTGSIALLLNAFLVDLPQQFH
LESITKTFQWLVDINNLVFKGSIPIVSLLFIYCLGVNIAKIYKVDTVSAGLVSLASFVIS
IGSTVTKSFPLANVGDVKLDQILQGIDNLAFDGKNLMVTIGNVIPGNHINARGYFTAMMI
GFLASIIFCKVMKKNWVIKLPDSVPPAIAKPFTSIIPGFMAMYIVAILTYVFHLLSNDLL
IDWVYKVLQTPLLGLSQSFFAVILMIFLNKLFWFFGLHGGNVLAPIMEGLFGVAMLANLD
AFQKGEPIPYIWTSGSFGAFVWFGGLGLVLAILIFSRNSHYRKVAKLGLAPVLFNIGEPV
NYGLPVVLNPLLFIPFVLSPVFMATVAYWATSWGLVSPVTQNVTWVMPPILYGFFSTAFD
WRAIILSVVCLIISVLTYFPFVKMADKTELS
>orf03238
(SEQ ID NO: 1653)
MNESNLESAMGLIMYGGEAKSNAMEAIQAAKKGDFSKANRRLADANAALLQAHKAQTEML
TREAQGEETSISLLMVHAQDHLMTSLTFVDLAKEVVEVYERFEKN
>orf03239
(SEQ ID NO: 1654)
MAKVTIMLACAAGMSTSLLVTKMQKAAEDKGLDAEIFAVPAPEAEEIVATKEVNVLLLGP
QVRYLLGDFQEKLKDRQIPVAVIPMTDYGMMNGSKVLDLAESLLD
>orf03240
(SEQ ID NO: 1655)
MKRLISANPSEILQMNAEELKQSILASEGRVVLSENVVTRETFVGDITNSEIARAFGADM
ILLNCVDVFEPKIYALDSSGDDVIHRLHQLVACPIGVNLEPIDPSAKMLEETQEIVAGRV
ASVETLKRIEELGFDFVCLTGNPGTGVSNREIIKAVQTAKENFSGLIIAGKMHGAGVNEP
VAELSVAEQLLEAGADVILVPAVGTVPAFHDQELREVVDLVHSKGGLVLSAIGTSQETSD
TDTIKEIALRNKICGVDIQHIGDAGYGGLATVDNIYALSKAIRGVRHTVSRLARSVNR
>orf03241
(SEQ ID NO: 1656)
MEKLLQEKLLPVAARLGNNKALVSIRDGITLTIPLLLIGSLLMVIASFPIPGWEQYLGDI
GVADYLWKGVDSSFGLLGLVASFGIAYFMARQYKVDGIPAGIVSLSSFITVIPFIRGEAG
AGMPTAFMASKGLFVAMILGLINGYIYQWFINHNIQIKMPDGVPPAVSKSFSAIIPGAVT
IVGWLIVYATLDKLSLPNLHEIAQVALGGPLGLLGNNVIGLLILIFLNSSFWFVGLHGGN
VVNAVMKPLWLANLDANKVAYQTGETLPNIFTSVFMDNFVFIGGGGATIGLVLALGYLAH
KKKASKQLKTLAPITVIPGLFNINEPAMFGVPIVLNILLLVPFILAPMFNLLVAWGAMAS
GLVPLTYTDPGWTMPPVISGLLATGSISGSLLQIVLIVLDVLLYLPFVIAIEKRFKLLED
>orf03242
(SEQ ID NO: 1657)
MTLSKKQLQLRAKILETVYTLGPISRIEIATKTGITPATTSSITNDLIKENILLELGEDE
HDTSVGRKKILLDIQAKRFYYIGCELSEKHFTFALGDNLGNILKEEKEIVTKQLIQEKGN
QLINQTLKQFLNNCSDYEIEAIGIALPGRYLDDYKITTNNPLWQHIDLEMIQSHFDKPLF
FSNNVNCMAIGKRLFSRQQNDPNFAYFHFARGMHCSYIYDGNIYGKGNLMIGEIGHTVVS
SEGEECSCGRKGCLQTFAGESWLIKKSKILYHQSPYSLLPSLVKNADDIDIQVILTAYQL
GDTGIITLIHQALLYLSQTILNISMMIDSQKIYLHSPLLTNQHIIQKLYSEMNYKPKLLY
NRLPEVIIEPYNDFTAAHSAIALCLYHTILHS
>orf03243
(SEQ ID NO: 1658)
MTIRFEEKVSTENAQFVCQWSNSLGKVFQEQWIGPRIPFPLTIQVFQDLEGILSIFEGQE
FVGLIQKIRLEDSNLHIGRFFINPQKQGQGLGSQALRKFVSLAFENRDIDSISLNVFEAN
QRAQNLYQKEGFEIV
>orf03261
(SEQ ID NO: 1659)
MPFKENLICQHRNHHCSVFFISLGLLHNIHIEIDISQTRASFLDLSDYLQAVLMILQKFC
QAIGLAQRLDLLQLHLLHLTRLLL
>orf03271
(SEQ ID NO: 1660)
MYLLLLVVKDHIALIDKEMHVWRPNCILRDLTNFFIKRNHIVTHKTNGSTTKR
>orf03272
(SEQ ID NO: 1661)
VLTLMNHFIKEIQGIPINHLTILIENSIFKLNLKNWIIG
>orf03274
(SEQ ID NO: 1662)
VDRTDEVSSKHCFEVADRTDEVSNHTYDKATLTRFEEFFEEYKGVPR
>orf03293
(SEQ ID NO: 1663)
VCQRMDARTCKTTIIAVHNVLTALQQTWIAVQLYQTK
>orf03294
(SEQ ID NO: 1664)
LHLGKSILSLPVKGKDLEFLVHLFVINHWIGFPSRTSTFCRCKVLNSME
>orf03295
(SEQ ID NO: 1665)
LEQTVIIANNPCELYWDNHLSFLSDSLLKQVIVHLKRICLDIHHDRGCSHVRNDTT
>orf03297
(SEQ ID NO: 1666)
LTDDGVLILVVDAGWRGNSCLQEQGCHHFRAILLCITWHFRSCTDKGHLTFKDIDQLRQF
VQTDTSDEISNLGNTAIVSRSHQTSFFIRIRHHGTELPNLEPTVVLGHTLLLVNHWPLAI
QLDPNAQDEKDGRS
>orf03298
(SEQ ID NO: 1667)
MLKMRKMGEVRTSKTKAKTQSKQRLKISEPFLLETSW
>orf03313
(SEQ ID NO: 1668)
MLEEGTKDQLAELTYPFGRGVNLSFGIKDVPKLYQKVMEANYPIYRLLTKRKFRVSDPYI
YPHKFAVLDPDGYFLRFSE
>orf03316
(SEQ ID NO: 1669)
MDQNLFNYNDEDIDSVIEYSHKLLNRKFSDVMEEYNRSLYKSYDDYNDRVVSEVQDKAIS
MKSKGQYGNYIEKYFYGYQPNSDSEADFEKIGVELKVTPFKINKNGTLSAKERLVLTILN
YMEENLEDFYSTHLWKKCAKILLLFYNGLIPNQTMKDYVIEKIFLYEWFEEDMAVILEDY
QKITDKIKNGKAHELSESDGNYLSTCTKGAGKGKDLRQQPFSHELAKQRAWELKSSYMTY
LINHKIFNQSDQESVLANFRGEKKSFTEVIAEKILSYKGFSEQELYDRFEVNSKAKGKNS
TLIRKILGLTGDLDKTKEFQKANMNLRVIRVDKNNLPKEDSPFKTYCFKELAATDSWESS
HVYNEIYNKRFLFVIFKEIEPKLFVLDSIKFWGFQDRQLEEIQRVWQETRQIISDGVKLT
QNGNKVSTNFPQSKINKILFTKLHATNTYYEIDKGKFVGKGSLSDTDELPDGRRITKHSF
WMPKKFIKEILDGNWD
>orf03317
(SEQ ID NO: 1670)
MKVLELFAGVGGFRIGLENADKQLFKTKWANQWEPSRKSQDAFEVYDYHFPNSKNINISI
SDITDEQFSKMDADMIVGGFPCQDYSVARSKKNEKGIEGKKGVLFWEIIRATEIIKPKYL
ILENVDRLLKAPSKQRGRDFAIMLTAFNNLGYSVEWRVINAAEYGRSQRRRRVFFFVYRN
DTVFAQKIDNLYEKNEEIFEDNRYDDYIFNQGLFAKQFPIKPIAVKNRHVFYELPNDIVE
VSDTFTGTVWNTGIMRRGKYYSIDTEPNYNGNPITLGEILQDESEVPEKYFLTDQSKLEK
FQYLRGPKKIERTSSDGHQYIYSEGGMSPYDDLNLPGRTMLTSEGTVNRSTHLLFVNNKY
RLITPIEAERLQDFPDDWTAKKKLSDDSIVEVSDKMRMFFMGNALVTEIVKEIAKFIKEI
D
>orf03318
(SEQ ID NO: 1671)
MDTFSFNGQYIVEFSCLKVVDRGLECHPIKSQRDNHQTTDLVT
>orf03320
(SEQ ID NO: 1672)
MGIAIVVERRVHYFGRHHNVTISHFFNFVIFKGRYSVKMKVFHRFLLIFQTTL
>orf03333
(SEQ ID NO: 1673)
MIACRHDICKSQKGLEHPFCIIRRLTRDFNQRPVCIVEANIFCLKITPQIITNMIVARTV
KSSKTGITLTTSMCKRDNHKITWFHRRNGFPSFFNNPNRFVSTIFMSSFRFWITVPP
>orf03341
(SEQ ID NO: 1674)
MLRQFRLGFFDVRMTECHLKWKERENFHDFLKFYCKDS
>orf03350
(SEQ ID NO: 1675)
MNMNKDQIAILNGADNLNLTLWMTLKEICKEGCKSFFPVRNTCRMLDIGIPYRLGLSLSN
SSVLNGMDV
>orf03370
(SEQ ID NO: 1676)
MHKLRIFVNQLCRRFGIILGPFLVLGFQVLTQELELAIFFDLREEVLLQVIPQVCHFCYL
RKEFTTLNQHELTSHDHVLTRHFQTHGLQG
>orf03382
(SEQ ID NO: 1677)
MLHMNLFFQPFFTNLCKTLATGCCVKTVMEWSSIATTIDFKIIE
>orf03383
(SEQ ID NO: 1678)
LDNRAKEWIMSTAQNQAIHLSNQGTQGFIDHLLGNTG
>orf03385
(SEQ ID NO: 1679)
LSLDFFPDDRSRSVTSNDNHFDILGQEKVDQLPSIFTNLLSRTGAIGRPRRISNIDDFFM
GKLAHELAHNGQAPDTRIQKTNWSIIHTVFFLVFFLIDRSL
>orf03394
(SEQ ID NO: 1680)
VSYGSHIFFASNCLKQIFGFLFKFSHLILLILVKASLI
>orf03397
(SEQ ID NO: 1681)
MTSLLTLENIHKTFEAGTVNENHVLKGLDLEVEEGDFISVIGGNGAGKSTLMNILAGNLS
VDEGDLLLAGKSIKNLSVRKRAKDIARVFQDPKMGTASRLTIEENMAIALRRGQKRGLGW
GVKEKDRIQFQEALKELNIGLENRLKVDTQYLSGGQRQALTLVMTDLMKPKLLLLDEHTA
ALDPKTSQMVMDLTQKIVEHHQWTTLMITHDMNHAIEYGNRLIMLYQGKIVVDVKGEEKK
HLTVEDLMHLFQKNSGQSLVSDELVLG
>orf03398
(SEQ ID NO: 1682)
MNFVLSSLSEGLLWSIVAIGDYLTFRILDIADMTAEGAFPLGAAVVVSQIQAGANPWLAT
LLALLAGMVAGLVSGMLHTKMKIPALLTGIVTLTGLYSINIKIMGSVPNLSLGDSATVFK
QLASLGLTNEGAVFSLSLVCFLLVCLVLTLLMKTEIGLVLRSTGDNIPMSEANGVNVDTM
KIVGYMISNGLIALCGSLFAQNDGFSDVTSGTGTIVVGLSSVIIAEVLIHDLTIGGRLLS
IGIGAIVYRLIILNIYEIPNLDQNLVRLFNAILLALVLFAPELQKRLKIRGLKLRNE
>orf03399
(SEQ ID NO: 1683)
MAEVDMVFVPTDNIILSTMETVKQVSIKHKVPVFGGSTEMIAVGGLYNYGTNYEELGRQT
ARMLIRVLKGEEPENIAVELPEKLELHTNQEMADALGIDISKLEGKE
>orf03400
(SEQ ID NO: 1684)
VDELAKQGYVEGENIEIDLQNAQGEQRNLKTISQQLAESSDVVLAIARPSAQSLANTTQT
TPVIFSAVTDPVSAKLVESREHPGGNVTGTSDQSSDAISTQINLIKKVLLKAKTIGILYT
QSEPNSVV
>orf03401
(SEQ ID NO: 1685)
MQTDQRSQEEPHYQEGASDFRTTFIMKLLLRKDKTKNRLDTI
>orf03402
(SEQ ID NO: 1686)
MLPILSPFSSPVNNISEFFKIFRKFFQEAFKVFQISPTKKVL
>orf03412
(SEQ ID NO: 1687)
MINVNQVSIEVKNTFKNWNFTSSIELTTFSKFSQSPTMT
>orf03414
(SEQ ID NO: 1688)
LIDVLFINSFIGRICFYCYRRIHATCLFLQLFSIVILNVAHTLKHSIFIVITFISRCRNF
IIVRILLENQFSRNQGIDNRVGQSRY
>orf03423
(SEQ ID NO: 1689)
MKIKEQTRKLAAGCSKHCFEVVDETDEVSNHTYGKVKLTWFEEIFEEYKKSSWNL
>orf03442
(SEQ ID NO: 1690)
LVEQLTFNQWVTGSSPVRVIYAGLAELADAPDLGSGA
>orf03443
(SEQ ID NO: 1691)
MSIVKSHSFSISLGIFNSFWNNIHTSECFNFLCKGKSNRSNSTISVNQMVFFINIQRFYC
FAIEDFCLLRI
>orf03444
(SEQ ID NO: 1692)
LNTLLPPDNLCLFTIYLTGFSCICINSYCHNFWEIFNQLFYQLS
>orf03451
(SEQ ID NO: 1693)
MGFSMKLIHDLNTHTTHSTAKMLHNVKAIKNDFSIRE
>orf03460
(SEQ ID NO: 1694)
MYNKVILIGRLTSTPELHKTNNDKSVARATIAVNRRYKDQNGEREVDFVNMVLWGRLAET
LASYATKGSLISVDGELRTRRFEKNGQMNYVTEVLVTGFQLLESRAQRAMRENNAGQDLA
DLVLEEEELPF
>orf03464
(SEQ ID NO: 1695)
MQFTRTAHHTKTLFTTKFTWENEIPFWHHSSRKRDNGFQPHTRIGSSCNDLYSLITCDCN
LADVEVVTIWMGYHLNNFTDNKLRFLIINNFFCKTFRLQLLVQTSDLLICQKDLTALCSF
K
>orf03469
(SEQ ID NO: 1696)
MILDSFFAFNCSGTMKVSTWVYDKGEWYYVSSSGSMIANDWVKDNGK
>orf03476
(SEQ ID NO: 1697)
MCTSINKKKKAVKPSFDLFFCFIFCKLTIVQVSVEATLRHQFLIVALLDDISIFHDQDQV
CISDG
>orf03484
(SEQ ID NO: 1698)
LHLRTCFVRQTNKLSPLINRTRLQFHQTILHYTLNQITSNRLGNIEFLIDIFNQDQVLVF
LAIIQKMHNLTLRPTHKFNAATFGFLLHHQVNLMTKTLKD
>orf03499
(SEQ ID NO: 1699)
MLEIWKYRPFVSEFWNDFKNNHDKQFVDSISLYLTLKDDDDPRIEEESEALENMILQYLG
EDDAS
>orf03507
(SEQ ID NO: 1700)
MAFNQFNRCIGLSIPTAPNVPGTIINRSYLHDATVPNNVREKT
>orf03523
(SEQ ID NO: 1701)
LTDFHDFKFIFFENLFKSRQLYLQSQNTVLSNLWLAT
>orf03533
(SEQ ID NO: 1702)
MKIMKKKYWTLAILFFCLFNNSVTAQEIPKNLDGNITHTQTSESFSESDEKQVDYSNKNQ
EEVDQNKFRIQIDKTELFVTTDKHLEKNCCKLELEPQINNDIVNSESNNLLGEDNLDNKI
KENVSHLDNRGGNIEHDKDNLESSIVRKYEWDIDKVTGGGESYKLYSKSNSKVSIAILDS
GVDLQNTGLLKNLSNHSKNYVPNKGYLGKEEGEEGIISDIQDRLGHGTAVVAQIVGDDNI
NGVNPHVNINVYRIFGKSSASPDWIVKAIFDAVDDGNDIINLSTGQYLMIDGEYEDGTND
FETFLKYKKAIDYANQKGVIIVAALGNDSLNVSNQSDLLKLISSRKKVRKPGLVVDVPSY
FSSTISVGGIDRLGNLSDFSNKGDSDAIYAPAGSTLSLSELGLNNFINAEKYKEDWIFSA
TLGGYTYLYGNSFAAPKVSGAIAMIIDKYKLKDQPYNYMFVKKILEETLPVKNGIKVLNI
PNVLRYDLNMLQLEYKNEQSWDSFIDNVNLIELEERIQTTIGIKQINTHNIITIAREGYS
QNYLPNTSENTYNSLQVSLVGVLLLFISMVNILWAKKSK
>orf03543
(SEQ ID NO: 1703)
VLKWCILRINHHISRKVDNFLEGTRAHIKGQAHTAWNPLEVPDVRYRSFQFDMSHTLTTN
FRTRYFNPTAVTNNSSVTNAFVLTTSTFPVFCRTKDHFIKESFTFWFQGTIIDCFRFFDF
SIRP
>orf03553
(SEQ ID NO: 1704)
MPWKELCHKLAPKVFKVIRIYSRENKKSPSNWAFCSFET
>orf03559
(SEQ ID NO: 1705)
VSVLFFCSYFSLSLEKGWFSSLISCKFMNQFLPFCWRQDSPWILTLAQDSITYH
>orf03564
(SEQ ID NO: 1706)
VTDENTRKVRLLVAFFSIVIGYILSSFFISLYHLWQEALRGLL
>orf03566
(SEQ ID NO: 1707)
LHVELIDSHKFNIGRTTCSLLSTTNICKRCQPSINHMS
>orf03567
(SEQ ID NO: 1708)
MLNTNRNELIGTSFLIFCVIYFKDLANIFRTTWNLYIIRQGHYKCQESHNQGRNDV
>orf03570
(SEQ ID NO: 1709)
VNKPILSDIDCHLTNSINLFLPDTQTGNLFWKFNGLIRLAHNHNIFRKKLSLSHFFNICY
DLFLGIGRV
>orf03571
(SEQ ID NO: 1710)
LKFSNFLGHLDIFSHDGLSLTVSLHQNSTGHATRYCFDR
>orf03584
(SEQ ID NO: 1711)
VDSLFLSLGEESNQEINLQESFSSTDCNPTLISPETTVAQGLCQDIIYRPFT
>orf03586
(SEQ ID NO: 1712)
VNPKSLGSFFLQDSKGFKELVLGHAKLSLPRIVHNVCPQFKNASRIITTRDDFWNACYSL
QMFNIFKGIQVNGRTQFTCIGVFLVWRVVGREHNLRTQKVQFMAHQKLYITRAVHTTTFF
LENFQNSWSWSSLNCKIFLKALVPRKSLVDGSCLLTNPLLIIQVKGSRELGNNRF
>orf03590
(SEQ ID NO: 768)
MDNLCLHNTWTDWTSIFKQAVVTEDDMTKQNDFFLGIIDAEFHNCLGNFAINESDMSKKI
TSHCVLCLVWPRQLDDLSQVMQHNPRIEQALIELRINFANSVCQTHHGRRMIGQARFKGM
VVGLGSWIGVEFLIILGVEISDNPLPDRIFNFENHLRHVVTNFLDINW
>orf03591
(SEQ ID NO: 1713)
LIDLRGIVINFSASFHVDNLTCGKGLNVMRLGIPELPINLATIILEGKG
>orf03604
(SEQ ID NO: 1714)
MDALVLQKNQETIQQIAVKIRFLDGHDYYSLIDIDNRRTNQTVFPFVNFEDIAF
>orf03605
(SEQ ID NO: 1715)
MAFFTEIPTRACLINLAITLHIVETCQGFNDLSLHLRVLAL
>orf03609
(SEQ ID NO: 1716)
MLLPLPFNTSKIKQIAMHSDLNQKEMIGHIFHDEDIF
>orf03614
(SEQ ID NO: 1717)
MKQTVKKLALVASIAATLGGGVSVASAAVQYPEGGVWTYGSGNGGAYSNYYHPSKYHSST
VVSRKTGSSDKGYAGAGGTSRAWIRTSWGEKVAFYYNV
>orf03643
(SEQ ID NO: 1718)
LVEQLTFNQWVTGSSPVRVIYAGLAELADAPDLGSGA
>orf03675
(SEQ ID NO: 1719)
MKELLNKAFFNKNKASLSKEVLLELQGKRLPVNLFLSKSLFQASL
>orf03690
(SEQ ID NO: 1720)
MMTKIKLTIDIIMPCRHITNIWIYKEEGRVNLFFYSQIFFDAIDERIIHNFNSKYHLSFF
SPVTGFSQIFDKTLACLG
>orf03695
(SEQ ID NO: 1721)
LHTSFRSSVGHSHTWHQDIVRPILFSRFNDSIVILWQNCPTFN
>orf03713
(SEQ ID NO: 1722)
MLEQARLKVEQQAIKNIQFLEQDLPKNPLEKEFDCLAVSRVLHHMPDLDAALSLFHQHLK
EDGKLIIADFTRTEANHHGFDLAELENKLIEHGFSSVHSQILYSAEDLFQGNHSEFFLIV
AQKSLA
>orf03714
(SEQ ID NO: 1723)
MKHDFNHKAETFDSPKNIFLANLVCQAAEKQIDLLSDKEILDFGGGTGLLALPLTPSQAG
>orf03716
(SEQ ID NO: 1724)
VWKKKKVKAGVLLYAVTIAAIFSLLLQFYLNRQIAHYQDYALNKEKLVAFAMAKRTKDKA
EQESGEQVFNLGQVSYQNKKTSLVTTVRTSKSQYEFLFPSVKIKEEKRDKKEEVATDSSE
KAEKKNQKRSLKRKRIPSQFNYNALNPE
>orf03718
(SEQ ID NO: 1725)
MVDLQSFFTRKYLNLNSVDAYLILPRLQGHLSYPQDFFLLQDFCFLLPIFLNLSQKEGRN
AGKDS
>orf03733
(SEQ ID NO: 1726)
MRIRNSPFDHILQTIFEFEDRTCQVTCRFEACSSICNDNWEFSQHIISVFQSPSCHTVCD
KSDVFCSFLFDKNFASLWIYVVTITDQLCIGMWQLVHGSNHTQFTVSQPTHSIVGMHPNT
RSSIDCFFGFIKSRV
>orf03734
(SEQ ID NO: 1727)
MSKSNRHTFARNCTNKVFHPITFWCKGNFIKQAICRFLPRMKLLNTRVSHISWILCPLKS
FCEIWTFIINPTNLSTCCFFIMVSKIFSDCKQLLISGC
>orf03736
(SEQ ID NO: 1728)
MQCTFNVVVHHIYTCISMNMSIHKTWGNAITCIVNHLSPFRNLLYMFPKLAVHKFQVTTS
TNSVWVEKLIRFNIVRHNVNLLKRLILQFIMSITL
>orf03750
(SEQ ID NO: 1729)
MKKRMLLASTVALSFAPVLATQAEEVLWTARSVEQIQNDLTKTDNKTSYTVQYGDTLSTI
AEALGVDVTVLANLNKITNMDLIFPETVLTTTVNEAEEVTEVEIQTPQADSSEEVTTATA
DLTTNQVTVDDQTVQVADLSQPIAEVTKTVIASEEVAPSTGTSVPEEQTTETTRPVEEAT
PQETTPAEKQETQASPQAASAVEVTTTSSEAKEVASSNGATAAVSTYQPEETKIISTTYE
APAAPDYAGLAVAKSENAGLQPQTAAFKEEIANLFGITSFSGYRPGDSGDHGKGLAIDFM
VPERSELGDKIAEYAIQNMASRGISYIIWKQRFYAPFDSKYGPANTWNPMPDRGSVTENH
YDHVHVSMNG
>orf03763
(SEQ ID NO: 1730)
VLAEADALVDAEAEALVDAEADALVLAEAEALVDADSDALVDAEADALVDAEAEALVDAD
SDADVLADTEAEALVDAEADALVLVDADVLALVDADVLADVLALVDADVLAEAEALVLAE
AEALVDAEAEALVDADSDAEVLAEADALVLAEADALVDAEAEALVDAEAEALVDAXXHSF
IN
>orf03764
(SEQ ID NO: 1731)
VRRLQHRQVLQLQRQPVRRLQRQPVRQPQQAPVLRLQQVLAPQPQHQQVLRSQRQPVPLN
PHQPVHRLQQVLAPQLQHQRVLQLSMNQCVGIRINQCIGFSKY
>orf03766
(SEQ ID NO: 1732)
VRRNPHQPVHRLQQVLVHQLQHQRVLRLQQAPVRLNPHQRLPQPQQVPVRQLQQVLVHQL
PHQQVLQLQRQPAPQPQQVPVRQLQQAQAPLSQRQPVRQLQXXXFHSLIN
>orf03772
(SEQ ID NO: 1733)
VTDENTRKVRLLVAFFSIVIGYILSSFFISLYHLWQEALRGLL
>orf03774
(SEQ ID NO: 1734)
MNXXALVDAEAEADVDAEAEALVDADAEALVLAEADALVDADSDADVLAEAEALVDAEAD
ALIDADSEADVLAEAEALVDAEADALVDAEAEALVLADAEALVDAEADALVDAEADALVD
AEADALVDAEAEALVDAEADALVLAEAEALVDAEADALVDADSDAEVLVLAEAEALVDAE
ADALVDADSDAEILAEADALVDAEAEALVLADSDALVNAEADVLAEADALVDADSEALVL
AEADALVLAEAEALVDAEAEGTGMRKLIH
>orf03777
(SEQ ID NO: 1735)
VLALVDADVLADVLALVDADVLAEAEALVLAEAEALVDAEAEALVDADSDAEVLAEADAL
VLAEADALVDAEAEALVDAEAEALVDAEAEALVDADSDADVLAEADALVDAEADALVLAE
ADALVLAEADALVDADSEALVDAEAEALVDAEAEALVDAEAEALVDAEAEALVDAEAEAD
VDAEAEALVDADAEALVLAEADALVDADSNADVLAEAEALVDAEXXIPFIN
>orf03794
(SEQ ID NO: 1736)
MKIKEQTRKLAVGCLKQCFEVVDRTDEVSSKYCFEVADGS
>orf03804
(SEQ ID NO: 1737)
LLGSFFSWTTKELMGIIFFNNFPTVHKNNMIGYISSKTYLIKLIKNSI
>orf03818
(SEQ ID NO: 1738)
MEKILLHNLNQTEFFINKAIGWTLRDYSKTNPTWVTCFIEKNKERMAELSIKEASKYL
>orf03819
(SEQ ID NO: 1739)
MSLADLLEELEAAKDSKKARSMEAYMRHQFSFLGIAVPERNKLYKNIFQKRKKQRLSIGI
LQTLAGKRILENTNMWLLTI
>orf03829
(SEQ ID NO: 1740)
MTTGWFQVNGRWYYAYSSGALAVNTTVDGYSVNYNGEWAQ
>orf03851
(SEQ ID NO: 1741)
MAFTTEELLNLGLTEEQAKSVFALRGKELNEDKSALETIKQERDSLKSQLQKAEEQVEHL
KSLENISAEQKDAIDKLQAEYDKYKNEAAAELAQTKKVSAISLALKDTNAFNPDKLMKFI
DVDAIQIDDNGKPQIDEVINGIKESDPHLFQAEESKPSPNIFPLR
>orf03853
(SEQ ID NO: 1742)
MGSSGEMRTRPAEELGVDTFYYSMKAMARPACSPLQGQIVTKGTGREIDGITIYSLLDYG
YGTAAGCLGIHCGHYLTPFIVGVHELPNLPDYLKNLTPEQAEENARIEAGQRGLERLIKT
HKERLHYAHTLQDDKMIQAERLKVRGYQTKIRNLINQHDFLTRDYRREKLYIS

In some embodiments, preferred 23F antigens are selected from the polypeptides orf01158 (SEQ ID NO: 1297), orf01305 (SEQ ID NO: 1309), orf01307 (SEQ ID NO: 1311), orf01631 (SEQ ID NO: 1343), orf01804 (SEQ ID NO: 1362), orf01807 (SEQ ID NO: 1364), orf02164 (SEQ ID NO: 1434), orf02189 (SEQ ID NO: 1451), orf02194 (SEQ ID NO: 1455), orf02219 (SEQ ID NO: 1466), orf02221 (SEQ ID NO: 1467), orf02224 (SEQ ID NO: 1470), orf02228 (SEQ ID NO: 1474), orf02242 (SEQ ID NO: 1484), orf02244 (SEQ ID NO: 1485), orf02246 (SEQ ID NO: 1486), orf02247 (SEQ ID NO: 1487), orf02652 (SEQ ID NO: 1491), and immunogenic fragments thereof.

### Example 3

### Pilus Adhesion

The pilus 2 mediates adhesion to alveolar epithelial cells A459. Dual-label staining shows that a pilus 2-positive strain adheres to the surface of the A549 cells and that the pilus (visualized using labeled anti-01287 antibody) is contacting the cells.

Furthermore, isogenic knockout mutants of the pilus are significantly impaired in host cell interaction. FIG. 2 shows that a pilus-negative strain (D39) fails to bind to A549 cells, in contrast to a pilus-positive strain (PN110). Knockout of the pilus in PN110 removes binding.

Incubation of A459 cells grown on glass coverslips with 01287 purified protein shows low level binding by confocal microscopy inspection. This observation was confirmed by incubating the protein with cells in suspension and quantifying the level of adherence by FACS analysis (FIG. 4). Pneumococcal pilus 1 RrgA subunit was used as positive control comparison, and green fluorescent protein (GFP) as a negative control.

Purified pilus was imaged by confocal microscopy. It was shown to adhere to A459 cells grown on glass coverslips. Furthermore, purified pilus seems to increase adherence to the respiratory cells when added to strains expressing the pilus type 2. This effect is probably due to the interaction of the purified pilus with both bacteria and A549 cells. Purified pilus does not increase adherence of isogenic knockout mutants of the pilus 2 to the respiratory cells (FIG. 3).

### Example 4

### Other Sequences from INV104B

An exemplary nucleic acid sequence for LepA Peptidase (orf 01289) is hereby provided:

(SEQ ID NO: 1743)
ATGCTGCTTAAAAAGAAACATAAGAAACCAGTAACACAAGTCAATCGGG
ATAAGTCTCCGCCGAGTGTCTGGGGAGATATCCTTTACTTAGTCAGTAA
ACTTCTGATGGTTGGATTTGTACTAGCCATCCTTTACTTTTTCGTCTTT
GGATTATTAAGATACAATGACGATGGCATGAAGCCCGCCTTAAAAGATG
GCGACTTGGTCGTCTATTATAGGTTGGATAAACGCTATTCGATTGGTGA
TTTGCTAGTCTATAGTTATAAAGGTAAGGAAAGAGTGGCGCGTGTCATA
GCAACCGAAGGAAGTACAATCGATATAAACGAAAATGGTCTCATCATCA
ACGGTTCTCCTCAACAAGAGCAAGATATCTACAAAGAAACGCTGCTCTA
TAAGGAAGGGGCAACCTTCCCGATGAAAGTOCCAGCAGGACAACTTTTT
GTCCTCGGGGACAATCGAACAACGGCTGTAGACAGTCGTGCTTTTGGAA
CCATCCCTATACAGGATACTCAAGGCAAAGTTGTAACAGTCATTAGAAG
ACGAGGCTTT

An exemplary amino acid sequence for LepA Peptidase is hereby provided:

(SEQ ID NO: 673)
MLLKKKHKKPVTQVNRDKSPPSVWGDILYLVSKLLMVGFVLAILYFFVF
GLLRYNDDGMKPALKDGDLVVYYRLDKRYSIGDLLVYSYKGKERVARVI
ATEGSTIDINENGLIINGSPQQEQDIYKETLLYKEGATFPMKVPAGQLF
VLGDNRTTAVDSRAFGTIPIQDTQGKVVTVIRRRGF

An exemplary nucleic acid sequence for sort-1 (orf01285) is hereby provided:

(SEQ ID NO: 1744)
ATGATGAAAACCAAGCGTGAGAAACCAAAAAAGAGTCTGTCTAGGCGTC
TCGTTCTTGCTGTGGATGGGGTGATCAATCACTTGCTGCTCATTTTTGC
AGCTTTGATCTTTCTCTTTGGTTTCTACGCCCTTTGGGATTCCAACCAA
GTCTACTCCTTAGCTTCGTCAAGTGAGTACGAAGCTTATCGACCTGTCA
CGACGCAACAGGATGAGCTGGCCAGTTTTTCAGGCTTCAGCAAACTCCA
AGAACTCAATCCCGAAGTCCTCGGTTGGATCAATGTCTATGGCACCAAT
ATCGACTATCCCTTAGTCCAAGCCAAGGACAATGAAAAGTATCTCAACA
AGGACTCCAAAGGTGAGTTTGCAGCGACAGGCGCTATCTTTCTCGATGC
ACGAAATAATCCTAAGTTCGAAGACTTTAATACCATTATCTACGGGCAC
CACGTAGAAAATGGGGTCATGTTTGGTGATGTGGCTAAGTTTGCTGATC
AGGAATTTTTTGACCAGCATCGTTACGGTAGTATATACTACAATGGTGT
GGAAAAAGGGCTCGAGATCTTTGAGATGTTGGAGGTTGATGCCTATGAC
TTTAACATCTATGATCCAGGAATACAGGGTGAGGACCGCCAGCAGGCCT
ATCTAGACCACCTGCTCTCAGTCGCCATGCACAAGCGGGATATCTCACT
CTCACCGAGTGATCGTATCATCCTACTCAGTACCTGTTTTCTCGATGTG
ACCAATGGTCGTCATATCGTAGTCGCAAAGATTACAGACACCGTCCCTA
AAAATACTTTCCATACAAAAAAATCAAAACCATTTCCATACAGTGTCTT
TGATGACTCGTCTCTTGGACGTTTCCTCTCATCAATCCCACTATGGATT
TGGTACCTTATCTTGTTTGTATTGTTCTTGCTCTTGATTTTCTTACTCC
TTGTCCTCTACTTGATCCTACGTCGTAGAAGAGAGAGTAAAAAAAATGC
AAGAAGCAGACCCTTTTACTGACTAAGGGTGAATAGAAA

An exemplary amino acid sequence for sort-1 is hereby provided:

(SEQ ID NO: 676)
MMKTKREKPKKSLSRRLVLAVDGVINHLLLIFAALIFLFGFYALWDSNQ
VYSLASSSEYEAYRPVTTQQDELASFSGFSKLQELNPEVLGWINVYGTN
IDYPLVQAKDNEKYLNKDSKGEFAATGAIFLDARNNPKFEDFNTIIYGH
HVENGVMFGDVAKFADQEFFDQHRYGSIYYNGVEKGLEIFEMLEVDAYD
FNIYDPGIQGEDRQQAYLDHLLSVAMHKRDISLSPSDRIILLSTCFLDV
TNGRHIVVAKITDTVPKNTFHTKKSKPFPYSVFDDSSLGRFLSSIPLWI
WYLILFVLFLLLIFLLLVLYLILRRRRESKKNARSRPFY

An exemplary nucleic acid sequence for sort-2 (orf01282) is hereby provided:

(SEQ ID NO: 1745)
ATGACGGTTCAAAAAAGAGCGCGATTTAAAAACGTATTTCTGGTATTCT
TCTGTGTTTTTGTAGCTCTTTTTAGTTGGCAGAGAGTAGTAGAAGCAAG
TGACTATGATCACTATAATCCTATTGAAAAGGATGCTTCGAGCACAGGT
TTTGAAACCCTACAGCACTTGAACAAAGATGTTTGCGGTTGGATTAGCC
TTGATGGGACCAAGGTAGACTATCCGCTTCTACAAAGTCAGGATAATGT
CAAATACCTTGACCGCAATGCCTTTGGCGATTATACGATAATGGGATCA
ATTTTTCTCGACTATCGCTTTAATCCCAACTTTACTGATTTTAATACGA
TCATCTACGGACACTCTATGGCTTCAGGGGCTATGTTCGGTGAGATTAA
GAAATTTGCTGATAAGGAATTCTTCGACCAGCATCGCTACGGTTCTATC
TACTACAATGGTCGAGAACGTGGTCTTGAAATTTTTGGGATTTTAGAAG
TGGATGCCTATGACACGGAGATTTATCGAACCTTGAGTTCCAAGGATGA
GGAACACCAGGCTTACTATCAATATCTGCTAAGTAAAGCCAAGTACAAG
CGAGATGTTTCCTTAACA

An exemplary amino acid sequence for sort-2 is hereby provided:

(SEQ ID NO: 1123)
MTVQKRARFKNVFLVFFCVFVALFSWQRVVEASDYDHYNPIEKDASSTG
FETLQHLNKDVCGWISLDGTKVDYPLLQSQDNVKYLDRNAFGDYTIMGS
IFLDYRFNPNFTDFNTIIYGHSMASGAMFGEIKKFADKEFFDQHRYGSI
YYNGRERGLEIFGILEVDAYDTEIYRTLSSKDEEHQAYYQYLLSKAKYK
RDVSLT

A number of embodiments of the inventive methods and compositions have been described. Nevertheless, it will be understood that various modifications may be made without departing from the spirit and scope of the invention.

TABLE 1
Pilus and Pilus II Screening Results

| | | | | Patient Information | | | | | | |
|---|---|---|---|---|---|---|---|---|---|---|
| Strain Identity & Pilus Presence | | | | | Age | | | Strain Source Information | | |
| Strain | Serotype | Pilus | Pilus II | Year | (yrs) | Sex | Diagnosis | Source | Country | Reference |
| 19FIJ | 19F | No | No | | | | | Iceland | | Same ST of |
| | | | | | | | | | | NCTC11906 |
| England14-9 | 14 | No | No | | | | | PMEN | England | PN93/872/B (ATCC |
| | | | | | | | | | | 700676) |
| PN137 | 14 | No | No | 1999 | 51 | F | meningitis | ISS | Italy - Arezzo | Same ST of |
| | | | | | | | | | | England14-9/mefA |
| PN099 | 14 | No | No | 1998 | 9 | M | meningitis | ISS | Italy - Reggio | erm |
| | | | | | mos. | | | | Emilia | |
| Spain14-5 | 14 | No | No | 1990 | | | | PMEN | Spain | MS22 (ATCC 700902) |
| CSR14-10 | 14 | No | No | 1987 | | | | PMEN | Slovakia | 87-029055 (ATCC |
| | | | | | | | | | | 700677) |
| Tennessee23F-4 | 23F | No | No | 1991 | 1 | F | bacteraemia | PMEN | USA | CS111 (ATCC 51916) |
| South Africa19A- | 19A | No | No | | | | | PMEN | South Africa | 51702 (ATCC 700904) |
| 13 | | | | | | | | | | |
| 67A | 8 | No | No | | | | Meningitis | Brazil | Brazil | |
| G54 | 19F | No | No | | | | | Lab strains | Italy | Complete - Glaxo |
| | | | | | | | | | | Wellcome |
| South Africa19A-7 | 19A | No | No | 1989 | | | | PMEN | South Africa | 17619 (ATCC 700674) |
| Spain23F-1 | 23F | No | No | 1984 | 67 | | pneumonia | PMEN | Spain | SP264 (ATCC 700669) - |
| | | | | | | | | | | Finishing - Sanger |
| Spain6B-2 | 6B | Yes^{a} | No | 1988 | | | other | PMEN | Spain | GM17 (ATCC 700670) |
| 1889 | 18C | No | No | 1996 | | | sepsis | ISS | Italy - Bergamo | (ST100 generally |
| | | | | | | | | | | associated to |
| | | | | | | | | | | serotypes 33F) |
| AP141 | 14 | No | No | 2002 | 38 | | sepsis | ISS | Italy - Bergamo | |
| AP173 | 14 | No | No | 2003 | 3 | | sepsis | ISS | Italy - Novara | |
| 6BIJ | 6B | Yes^{a} | No | | | | | Iceland | | |
| PT051 | 14 | Yes^{b} | No | 2001 | 49 | | sepsis | ISS | Italy - Torino | Same ST of Spain 9V-3/ |
| | | | | | | | | | | erm |
| Spain9V-3 | 9V | Yes^{c} | No | 1993 | | | | PMEN | France | TL7/1993 (ATCC |
| | | | | | | | | | | 700671) |
| Poland23F-16 | 23F | Yes^{a} | No | 1999 | 13 | F | LRTI | PMEN | Poland | 178 (ATCC BAA-343) |
| PB011 | 6B | Yes^{d} | No | 2001 | 11 | | meningitis | ISS | Italy - Bari | |
| | | | | | mos. | | | | | |
| PB001 | 3 | No | No | 2001 | | | meningitis | ISS | Italy - Bari | erm |
| PT131 | 3 | No | No | 2002 | 34 | | pneumonia | ISS | Italy - Novara | |
| 70A | 3 | No | no | | | | Meningitis | Brazil | Brazil | |
| OXC141 | 3 | No (by | no | | | | | Sanger | | Finishing - Sanger |
| | | BLAST) | | | | | | | | |
| South Africa6B-8 | 6B | No | No | 1990 | | | | PMEN | South Africa | 50803 (ATCC 700675) |
| AP/PT108 | 7F | No | Yes | 2003 | 3 | | sepsis | ISS | Italy - Cuneo | erm |
| 32/14 | 7F | No | Yes | 1999 | <5 | | carriage | ISS | Italy - Roma | |
| PN195 | 7F | No | Yes | 2002 | 44 | | meningitis | ISS | Italy - Napoli | |
| 86A | 7F | No | Yes | | | | Meningitis | Brazil | Brazil | |
| 16117 | 18C | No | No | | 10 | | Carriage | Brazil | Brazil | |
| | | | | | mos. | | | | | |
| 6054 | 18C | No | No | | 6 | | Carriage | Brazil | Brazil | |
| 19135 | 18C | No | No | | 10 | | Carriage | Brazil | Brazil | |
| TIGR4 | 4 | Yes^{c} | No | | 30 | M | meningitis | Lab strains | Norway- | JNR.7/87 (ATCC BAA- |
| | | | | | | | | | Kongsvinger | 334) - Complete -TIGR |
| INV104B | 1 | No (by | Yes | | | | | Sanger | | Finishing - Sanger |
| | | BLAST) | | | | | | | | |
| PNS28 | 14 | No | No | 2001 | | | sepsis | ISS | Italy - Perugia | Same ST of Clone 32 |
| Taiwan19F-14 | 19F | Yes^{c} | Yes | 1997 | | | meningitis | PMEN | Taiwan | TW31 (ATCC 700905) |
| Taiwan23F-15 (**) | 23F | Yes^{e} | No | 1997 | | | bacteraemia | PMEN | Taiwan | TW17 (ATCC 700906) |
| Hungary19A-6 | 19A | Yes^{a} | No | 1989 | | | | PMEN | Hungary | HUN663 (ATCC |
| | | | | | | | | | | 700673) |
| Finland6B-12 | 6B | Yes^{a} | No | 1987 | | | | PMEN | Finland | 43362 Fi10 (ATCC |
| | | | | | | | | | | 700903) |
| SP307 | 5 | No | No | 2000 | 3 | | sepsis | ISS | Italy - Bergamo | |
| PT075 | 5 | No | No | 2001 | 61 | | sepsis | ISS | Italy - Torino | |
| 96A | 5 | No | no | | | | Meningitis | Brazil | Brazil | |
| PN57 | 1 | No | No | 1997 | | | meningitis | ISS | Italy - Roma | Same ST of INV1871 |
| P1031 | 1 | No | No | 2002 | 45 | F | Meningitis | Ghana | Ghana | |
| P1074 | 1 | No | No | 2003 | 70 | M | Meningitis | Ghana | Ghana | |
| PN110 | 1 | No | Yes | 1998 | 5 | | meningitis | ISS | Italy - Lecco | |
| PB013 | 1 | No | Yes | 2002 | | | sepsis | ISS | Italy - Bari | erm |
| SPPD | 1 | No | Yes | 2005 | | | bilateral | Padova | Italy-North | (isolated from from BE; |
| | | | | | | | pneumonia + | | | patient's sera available) |
| | | | | | | | sepsis | | | |
| D39 | 2 | No | No | 1916 | | | pneumonia | Lab strains | Caucasian | NCTC 7466 |
| R6 | cps- | No | No | 1930 | | | | Lab strains | USA | (ATCC BAA-255) - |
| | | | | | | | | | | Complete - Eli Lilly |
| P1054 | 1 | No | No | 2001 | 32 | F | Meningitis | Ghana | Ghana | |
| 1IJ | 1 | No | No | | | | | Iceland | | |
| 2010 | 34 | No | No | | 3 | | Carriage | Brazil | Brazil | |
| 30A | 3 | Yes?^{b} | no | | | | Meningitis | Brazil | Brazil | |
| 3IJ | 3 | No | No | | | | | Iceland | | |
| PN95 | 3 | No | No | 1998 | 65 | | meningitis | ISS | Italy - Milano | |
| PNS32 | 14 | No | No | 2001 | 32 | | sepsis | ISS | Italy - Napoli | Same ST of |
| | | | | | | | | | | Sweden 15A-25/erm |
| 27/13 | 14 | No | No | 1999 | <5 | | carriage | ISS | Italy - Roma | |
| 6AIJ | 6A | No | No | | | | | Iceland | | |
| 117 | 4 | Yes^{b} | no | | | | Meningitis | Brazil | Brazil | |
| JJA | 14 | No | no | | | | Meningitis | Brazil | Brazil | |
| 279A | 14 | No | no | | | | Meningitis | Brazil | Brazil | |
| P1040 | 14 | No | No | 2002 | 0 | M | meningitis | Ghana | Ghana | |
| P1059 | 6A | No | No | 2002 | 47 | F | meningitis | Ghana | Ghana | |
| P1075 | 14 | No | No | 2003 | 8 | M | meningitis | Ghana | Ghana | |
| P1076 | 14 | No | No | 2003 | 0 | F | meningitis | Ghana | Ghana | |
| P1077 | 14 | No | No | 2003 | 54 | M | meningitis | Ghana | Ghana | |
| P1083 | 38 | Yes^{f} | No | 2003 | 58 | M | meningitis | Ghana | Ghana | |
| P1086 | 4 | No | No | 2002 | | | meningitis | Ghana | Ghana | |
| P1095 | 4 | No | No | 2004 | | | | Ghana | Ghana | |
| P1101 | 38 | No | No | 2004 | | | | Ghana | Ghana | |
| P1104 | 4 | No | No | 2004 | | | | Ghana | Ghana | |
| PJ1466 | 7F | no | Yes | | | | | Sweden | Sweden | |
| PJ176 | 14 | no | No | | | | | Sweden | Sweden | |
| PJ1354 | 1 | no | Yes | | | | | Sweden | Sweden | |
| I101 | 3 | no | No | | | | | Sweden | Sweden | |
| P1022 | 3 | No | No | 2001 | 1 | M | meningitis | Ghana | Ghana | |
| P1068 | 10F | No | No | 2003 | 42 | M | meningitis | Ghana | Ghana | |
| RP1554 | 9V | yes^{c} | No | | 68 | | | Sweden | Sweden | |
| RP3718 | 9V | yes^{b} | No | | 4 | | | Sweden | Sweden | |
| AP207 | 9V | Yes | No | 2003 | 71 | | meningitis | ISS | Italy | |
| PT052 | 9V | Yes | No | 2001 | 74 | | meningitis | ISS | Italy | |
| PN131 | 24F | Yes | No | 1998 | 72 | M | meningitis | ISS | Italy | |
| AP062 | 9V | Yes | No | 2003 | 91 | | | ISS | Italy | |
| AP233 | 6B | Yes | No | 2003 | 82 | | | ISS | Italy | |
| PT134 | 6B | Yes | No | 2003 | 44 | | | ISS | Italy | |
| PN6 | 6B | Yes | No | 1996 | | | meningitis | ISS | Italy | |
| PN20 | 6B | Yes | No | 1997 | 1 | M | meningitis | ISS | Italy | |
| PN68 | 6B | Yes | No | 1997 | 65 | F | meningitis | ISS | Italy | |
| PN126 | 6B | Yes | No | 1998 | 3 | M | meningitis | ISS | Italy | |
| SP95 | 6B | Yes | No | 1999 | 4 | F | meningitis | ISS | Italy | |
| AP174 | 6B | Yes | No | 2003 | 18 | | | ISS | Italy | |
| PN217 | 6B | No | No | 2003 | | | meningitis | ISS | Italy | |
| 1404 | 6B | Yes | No | 1999 | | F | carriage | ISS | Italy | |
| Pn102 | 6B | Yes | No | 1998 | 2 | F | meningitis | ISS | Italy | |
| PB018 | 6B | Yes | No | 2001 | | | | ISS | Italy | |
| PN218 | 19F | No | No | 2003 | 58 | | meningitis | ISS | Italy | |
| AP235 | 23F | No | No | 2003 | 63 | | | ISS | Italy | |
| PGX1416 | 19F | Yes | Yes | | | | | ISS | | proviene dalla GSK |
| SME15 | 35B | Yes^{f} | No | | | | | Sweden | Sweden | |
| PJ1423 | 4 | Yes | No | | | | | Sweden | Sweden | |

Pilus Type: ^{a}6B/c;
^{b}T4;
^{c}T4/c;
^{d}6B;
^{e}23F/c;
^{f}23F

1. An isolated pilus encoded by the Streptococcus pneumoniae pilus II island (INV104B).

2. The pilus of claim 1, wherein the pilus comprises a sortase.

3. The pilus of claim 1, wherein the pilus comprises a LPXTG cell wall anchored protein.

4. The pilus of claim 1, wherein the pilus has been separated from cells by enzymatic digestion or mechanical shearing.

5. The pilus of claim 1, wherein the mechanical shearing comprises ultrasonication.

6. The pilus of claim 1, wherein the pilus is substantially free of bacterial cells.

7. An immunogenic composition comprising one or more pili of claim 1.

8. A method of producing the pilus of claim 1, the method comprising subjecting a bacterial cell that produces the pilus to enzymatic digestion or mechanical shearing and isolating the pilus from the cell.

9. An isolated Streptococcus pneumoniae sortase, wherein the sortase is selected from the group consisting of SEQ ID NO:282, SEQ ID NO:1386, SEQ ID NO:676, or SEQ ID NO:1123.

10. An isolated Streptococcus pneumoniae LPXTG cell wall anchored protein, wherein the LPXTG cell wall anchored protein is selected from the group consisting of SEQ ID NO:2, SEQ ID NO:4, SEQ ID NO:6, SEQ ID NO:7, SEQ ID NO:8, or SEQ ID NO:9.

11. A method of isolating pili encoded by the Streptococcus pneumoniae pilus II island (INV104B) comprising:
subjecting bacterial cells that produce pili encoded by the Streptococcus pneumoniae pilus II island (INV104B) to enzymatic digestion or mechanical shearing; and
isolating the pili from the cells.

12. The method of claim 11, wherein the mechanical shearing involves ultrasonication.

13. The method of claim 11, wherein the enzymatic digestion is performed using mutanolysin.

14. The method of claim 11, wherein the isolating comprises one or more density gradient centrifugations.

15. The method of claim 11, wherein the isolating comprises reducing polydispersity.

16. The method of claim 15, wherein polydispersity is reduced by separating components by size.

17. (canceled)

18. (canceled)

19. (canceled)

20. (canceled)

21. (canceled)

22. (canceled)

23. (canceled)

24. An immunogenic composition comprising a purified Streptococcus pneumoniae pilus II island (INV104B) polypeptide in oligomeric form.

25. The immunogenic composition of claim 24, wherein the oligomeric form is a hyperoligomer.

26. The immunogenic composition of claim 24, wherein the polypeptide is a fragment of a LPXTG cell wall anchored protein encoded by the Streptococcus pneumoniae pilus II island (INV104B).

27. The immunogenic composition of claim 26, wherein the fragment is at least 100 contiguous amino acid residues in length.

28. The immunogenic composition of claim 26, wherein the fragment is at least 50 contiguous amino acid residues in length.

29. The immunogenic composition of claim 26, wherein the fragment is at least 20 contiguous amino acid residues in length.

30. The immunogenic composition of claim 26, wherein the fragment retains the ability to covalently attach to a peptidoglycan cell wall.

31. The immunogenic composition of claim 26, wherein the fragment retains the ability to cross-link to another fragment or protein through an LPXTG motif.

32. The immunogenic composition of claim 24, wherein the polypeptide comprises two or more fragments of LPXTG cell wall anchored proteins encoded by the Streptococcus pneumoniae pilus II island (INV104B).

33. A method of inducing an immune response against Streptococcus pneumoniae, the method comprising administering an effective amount of pili encoded by the Streptococcus pneumoniae pilus II island (INV104B) to a subject.

34. The method of claim 33, wherein the pili are isolated.

35. The method of claim 33, wherein the subject is human.

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. An isolated pilus or pilus-like multimer comprising a polypeptide comprising the amino acid sequence of a pilus protein encoded by the Streptococcus pneumoniae pilus II island (INV104B) with up to 30 amino acid substitutions, insertions, or deletions.

58. The pilus or pilus-like multimer of claim 57 with up to 20 amino acid substitutions, insertions, or deletions.

59. The pilus or pilus-like multimer of claim 57 with up to 10 amino acid substitutions, insertions, or deletions.

60. The pilus or pilus-like multimer of claim 57 with up to 5 amino acid substitutions, insertions, or deletions.

61. (canceled)

62. (canceled)

63. The polypeptide of claim 57, wherein the protein is a LPXTG cell wall anchored protein.

64. A polypeptide comprising the amino acid sequence of one or more LPXTG cell wall anchored proteins encoded by the Streptococcus pneumoniae pilus II island (INV104B), or immunogenic fragments thereof.

65. The polypeptide of claim 64, wherein the polypeptide comprises the amino acid sequences of two or more LPXTG cell wall anchored proteins, or immunogenic fragments thereof.

66. A purified polypeptide consisting of an amino acid sequence selected from the group consisting of SEQ ID NO:2, SEQ ID NO:4, SEQ ID NO:6, SEQ ID NO:7, SEQ ID NO:8 and SEQ ID NO:9.

67. A purified polypeptide comprising at least ten consecutive residues of an amino acid sequence selected from the group consisting of: SEQ ID NO:2, SEQ ID NO:4, SEQ ID NO:6, SEQ ID NO:7, SEQ ID NO:8 and SEQ ID NO:9.

68. A purified polypeptide consisting of an amino acid sequence which comprises a sequence at least 85% identity to an amino acid sequence selected from the group consisting of: SEQ ID NO:2, SEQ ID NO:4, SEQ ID NO:6, SEQ ID NO:7, SEQ ID NO:8 and SEQ ID NO:9.

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. A purified polypeptide, the amino acid sequence of which is at least 85% identical to a sequence selected from the group consisting of SEQ ID NO: 29 through SEQ ID NO:1742, or an immunogenic fragment thereof.

97. (canceled)

98. (canceled)

99. The purified polypeptide of claim 96, wherein the sequence is selected from the group consisting of SEQ ID NO: 29 through SEQ ID NO:1742.

100. A purified polypeptide, the amino acid sequence of which is at least 85% identical to a sequence selected from the group consisting of SEQ ID NO: 53, SEQ ID NO: 65, SEQ ID NO: 70, SEQ ID NO: 99, SEQ ID NO: 104, SEQ ID NO: 117, SEQ ID NO: 135, SEQ ID NO: 177, SEQ ID NO: 178, SEQ ID NO: 198, SEQ ID NO: 235, SEQ ID NO: 236, SEQ ID NO: 237, SEQ ID NO: 242, SEQ ID NO: 247, SEQ ID NO: 248, SEQ ID NO: 250, SEQ ID NO: 251, SEQ ID NO: 252, SEQ ID NO: 253, SEQ ID NO: 433, SEQ ID NO: 439, SEQ ID NO: 444, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 581, and SEQ ID NO: 593, or an immunogenic fragment thereof.

101. (canceled)

102. (canceled)

103. A purified polypeptide, the amino acid sequence of which is at least 85% identical to a sequence selected from the group consisting of SEQ ID NO: 626, SEQ ID NO: 628, SEQ ID NO: 629, SEQ ID NO: 630, SEQ ID NO: 631, SEQ ID NO: 632, SEQ ID NO: 639, SEQ ID NO: 645, SEQ ID NO: 747, SEQ ID NO: 751, SEQ ID NO: 752, SEQ ID NO: 783, SEQ ID NO: 786, SEQ ID NO: 787, SEQ ID NO: 810, SEQ ID NO: 812, SEQ ID NO: 813, SEQ ID NO: 824, SEQ ID NO: 831, SEQ ID NO: 842, SEQ ID NO: 847, SEQ ID NO: 875, SEQ ID NO: 876, SEQ ID NO: 879, SEQ ID NO: 880, SEQ ID NO: 882, SEQ ID NO: 913, SEQ ID NO: 914, SEQ ID NO: 925, SEQ ID NO: 926, SEQ ID NO: 947, SEQ ID NO: 948, SEQ ID NO: 968, SEQ ID NO: 987, SEQ ID NO: 988, SEQ ID NO: 990, SEQ ID NO: 992, SEQ ID NO: 1003, SEQ ID NO: 1007, SEQ ID NO: 1008, SEQ ID NO: 1036, SEQ ID NO: 1082, SEQ ID NO: 1120, and SEQ ID NO: 1123, or an immunogenic fragment thereof.

104. (canceled)

105. (canceled)

106. A purified polypeptide, the amino acid sequence of which is at least 85% identical to a sequence selected from the group consisting of SEQ ID NO: 1297, SEQ ID NO: 1309, SEQ ID NO: 1311, SEQ ID NO: 1343, SEQ ID NO: 1362, SEQ ID NO: 1364, SEQ ID NO: 1434, SEQ ID NO: 1451, SEQ ID NO: 1455, SEQ ID NO: 1466, SEQ ID NO: 14678, SEQ ID NO: 1470, SEQ ID NO: 1474, SEQ ID NO: 1484, SEQ ID NO: 1485, SEQ ID NO: 1486, SEQ ID NO: 1487, and SEQ ID NO: 1491, or an immunogenic fragment thereof.

107. (canceled)

108. (canceled)

109. A polynucleotide that encodes a polypeptide of claim 64, or immunogenic fragment thereof.

110. (canceled)

111. A method of inducing an immune response against Streptococcus pneumoniae, the method comprising administering an effective amount of a polypeptide of claim 64 to a subject.

112. An immunogenic fragment of a LPXTG cell wall anchored protein encoded by the Streptococcus pneumoniae pilus II island (INV104B).

113. An isolated polynucleotide encoding a polypeptide comprising an amino acid sequence selected from the group consisting of SEQ ID NO:2, SEQ ID NO:4, SEQ ID NO:6, SEQ ID NO:7, SEQ ID NO:8 and SEQ ID NO:9.

114. An isolated nucleic acid consisting of a polynucleotide sequence selected from the group consisting of SEQ ID NO:1, SEQ ID NO:3, SEQ ID NO:5, a degenerate variation of SEQ ID NO:1, a degenerate variation of SEQ ID NO:3 and a degenerate variation of SEQ ID NO:5.

115. An isolated nucleic acid comprising a sequence that hybridizes under stringent conditions to a hybridization probe, wherein the nucleotide sequence of the probe is selected from the group consisting of SEQ ID NO:1, SEQ ID NO:3, SEQ ID NO:5, the complement of SEQ ID NO:1, the complement of SEQ ID NO:3 and the complement of SEQ ID NO:5.

116. An isolated nucleic acid comprising a sequence that encodes a polypeptide comprising an amino acid sequence which is at least 85% identical to an amino acid sequence selected from the group consisting of: SEQ ID NO:2, SEQ ID NO:4, SEQ ID NO:6, SEQ ID NO:7, SEQ ID NO:8 and SEQ ID NO:9.

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. An isolated nucleic acid comprising a sequence that encodes a polypeptide the amino acid sequence of which is at least 85% identical to a sequence selected from the group consisting of SEQ ID NO: 29 through SEQ ID NO:1742.

139. (canceled)

140. (canceled)

141. The isolated nucleic acid of claim 138, wherein the sequence is selected from the group consisting of SEQ ID NO: 29 through SEQ ID NO:1742.

142. A method of inducing an immune response against Streptococcus pneumoniae, the method comprising administering an affective amount of an immunogenic fragment of a LPXTG cell wall anchored protein encoded by the Streptococcus pneumoniae pilus II island (INV104B) to a subject.

143. The method of claim 142, wherein the subject is human.

144. (canceled)

145. (canceled)

146. (canceled)

147. (canceled)

148. (canceled)

149. (canceled)

150. (canceled)

151. (canceled)

152. A method of isolating pili encoded by the Streptococcus pneumoniae pilus II island (INV104B), the method comprising:
subjecting Streptococcus pneumoniae cells that produce pili encoded by the Streptococcus pneumoniae pilus II island (INV104B) to ultrasonication or digestion with a lytic enzyme;
separating non-cellular components by density gradient centrifugation; and
isolating pili encoded by the Streptococcus pneumoniae pilus II island (INV104B).

153. The method of claim 152, wherein the lytic enzyme is mutanolysin.

154. The method of claim 152, wherein the non-cellular components are separated using density gradient centrifugation.

155. The method of claim 152, wherein the Streptococcus pneumoniae cells that produce pili encoded by the Streptococcus pneumoniae pilus II island (INV104B) are Streptococcus pneumoniae TIGR4 cells.

156. The method of claim 152, wherein the method further comprises degrading nucleic acids with a nuclease.

157. The method of claim 152, wherein the method further comprises reducing polydispersity by separating the pili encoded by the Streptococcus pneumoniae pilus II island (INV104B) by size using gel filtration chromatography.
